(19) United States
(12) Patent Application Publication
(10) Pub. No.: US 2018/0340174 A1
(43) Pub. Date: Nov. 29, 2018

(54) METHOD FOR IDENTIFICATION OF MOLECULES WITH DESIRED CHARACTERISTICS

(71) Applicants: Mikkel Dybro Lundorf, (US); Henrik Pedersen, (US); NANOCORE APS, Roskilde (DK)

(72) Inventors: Mikkel Dybro Lundorf, Roskilde (DK); Henrik Pedersen, Bagsværd (DK); Tore Dehli, Odense N (DK)

(73) Assignee: NANOCORE APS, Roskilde (DK)

(21) Appl. No.: 15/525,681

(22) PCT Filed: Nov. 11, 2015
(86) PCT No.: PCT/DK2015/050343

§ 371 Date: May 10, 2017

Related U.S. Application Data
(60) Provisional application No. 62/077,933, filed on Nov. 11, 2014.
(60) Provisional application No. 62/202,318, filed on Aug. 7, 2015.

(30) Foreign Application Priority Data
Nov. 11, 2014 (DK) PA 2014 00655

(51) Int. Cl. C12N 15/10

(52) U.S. Cl. CPC C12N 15/1068; G01N 30/02; C12N 15/1065

(57) ABSTRACT

A method for identifying molecules with desired characteristics such as high affinity for a surface or material is described. A particularly useful method covered by the present invention allows identification of molecules which bind a material with high affinity in the presence of fluid or soluble polymers, such that said molecules can be used to produce a composite in which they efficiently anchor a material in a matrix comprising solid forms of a polymer. Compositions/kits useful for identification of molecules with desired characteristics are also described.

### FIELD OF THE INVENTION

The present invention relates to methods for identifying one or more molecules with desired characteristics such as a desired affinity for a surface or a material or such as affinity for a specific site on a surface or material. Furthermore, the present invention relates to kits/compositions useful for identifying molecules with desired characteristics. In addition, the present invention relates to the use of said identified molecules to modulate a material or surface, such as to modulate one or more characteristics of a material or surface. For example, said identified molecules of the present invention may be used to interact with, stabilize, coat, bind to, purify, enhance a characteristic of, protect, enable the production of, assemble, improve the production of, or improve the processing of, a surface or material.

### BACKGROUND OF THE INVENTION

There is great interest in the development of surfaces or materials with new or improved characteristics. Said characteristics may be controlled at the nanoscale or molecular level. Many materials are composites (composed of components—typically a matrix, such as a polymer, and a filler/additive, such as carbon nanotubes or graphene) and it is critical to control the interaction of the components at the molecular level. For example, fullerenes such as carbon nanotubes and graphene may be added as fillers to improve characteristics such as Young's modulus and/or tensile strength of materials comprising a polymer (e.g. plastic) matrix. Often, a characteristic of a filler is not efficiently transferred to (or manifest in) the resulting composite material unless the interaction of matrix and filler is efficient at the molecular level. To control, e.g. optimize, said interaction it is necessary to identify molecules with desired characteristics such as a high binding affinity to a filler and/or high binding affinity to a polymer. Thus, methods for systematically and efficiently identifying molecules with desired characteristics are needed. The present invention provides such methods.

### SUMMARY OF THE INVENTION

It has been found by the present inventors that molecules with desired characteristics, such as a high affinity to an inorganic material in a polymer composite, can be identified by methods which include conditions which mimic conditions in said composite.

So, in a first aspect the present invention relates to a method for identification of molecules with desired characteristics, comprising the steps of:

A. Providing a library of molecules of the general composition M(n)-L(k)-T(s):

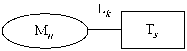

where n=0 to 1E+9, k=0 to 1E+9, and s=0 to 1E+9, such that n+k+s is at least 1,
and where M is a library molecule, T is a tag that may be used to identify the library molecule M to which it is attached, and L is a linker that connects M and T;
B. Providing a medium;
C. Providing a surface or material;
D. Combining said library, medium, surface and/or material;
E. Optionally performing one or more manipulations of said library, medium, surface and/or material;
F. Partitioning or isolating a fraction of said library;
G. Optionally amplifying or copying the whole or a part of one or more molecules;
H. Optionally identifying one or more characteristics of one or more library molecules;
wherein
steps A, B, C, D, E, F, G, and H are performed simultaneously or sequentially in any possible order; and
wherein steps A, B, C, D, E, F, G, and H are performed one or more times.

In a second aspect the present invention relates to a kit for use in the method according to the invention comprising fullerenes, such as carbon nanotubes and/or graphene, and a molecule library.

In a third aspect the present invention relates to a use of molecules identified by the method according to the invention to modulate a material or surface.

### Definitions

### Analog

The term, “analog” as used herein, shall be defined as an entity which shares one or more characteristics with another entity. For example, a nucleotide analog is an entity which can hybridize to another nucleotide. As another example, an oligomer may be an analog of a polymer analog, e.g. tri-ethylene is an analog of polyethylene.

### Detergent

The term, “detergent” as used herein, refers to a surfactant or a mixture of surfactants.

Display oligo

The term, “display oligo” as used herein, refers to an entity, which comprises a linker and an oligomer, such as an oligonucleotide.

Electrical resistivity

The term, “electrical resistivity” as used herein, refers to resistivity, specific electrical resistance, or volume resistivity and is a measure of how strongly a material opposes the flow of electric current. A low resistivity indicates a material that readily allows the movement of electric charge. The SI unit of electrical resistivity is the ohm metre [Ωm].

### Elution

The term, “elution” as used herein, refers to the process whereby molecules are partitioned from a material or surface, for example when a fraction of a molecule library bound to a surface is removed from said surface.

### Fluid

The term, “fluid” as used herein, refers to a substance that continually deforms (flows) under an applied shear stress. All gases are fluids, but not all liquids are fluids.

Hydrogen bond acceptor (HBA)

The term, “hydrogen bond acceptor” as used herein, refers to chemical moiety which can bind to hydrogen, such as fluorine, nitrogen, oxygen, sulfur.

Hydrogen bond donor (HBD)

The term, “hydrogen bond donor” as used herein, refers to chemical moiety which can donate a hydrogen atom such as hydrogens bonded to fluorine, oxygen, or nitrogen.

### Inorganic

Inorganic structural entities or materials shall mean any material or entity except those comprising carbon and at least one other element. Inorganic SEs thus include carbon nanotubes (CNTs), graphene sheets (GSs), other fullerenes, and carbon fibres (CFs).

### Library

The term, “library” is used herein interchangeably with “molecule library” or “library of molecules” meaning a collection or plurality of molecules.

### Ligand

The term “ligand” as used herein shall mean an entity capable of binding covalently or non-covalently to a material, where said entity is a molecule composed of atoms, which binds said material, or which connects at least two atoms that said material.

### Linker

The term “linker” as used herein describes an entity which links one or molecules or molecule fragments to one or more tags.

### Material

The term, “material” as used herein, describes anything made of matter. The material may be in a solid state (such as ice) or for all practical purposes solid (e.g. as glass).

### Medium

The term, “medium” as used herein, shall be defined as a liquid or a gas wherein molecules such as organic solvents or materials may be dispersed or dissolved. A “medium” as used herein may comprise solvents, salts, buffering components, such as pH-buffering components, or fluid polymers.

### Mimic

The term, “mimic” as used herein, refers to an entity which shares a characteristic with another entity. For example a nucleotide mimic is an entity which can hybridize to a nucleotide, such as the ability to hydrogen-bond to another nucleotide.

### Molecule

The term, “molecule” is used herein interchangeably with “library molecule”, and refers to a substance composed of two or more atoms; a group of like or different atoms held together by chemical forces, e.g. chemical bonds.

Multi-walled nanotubes

Multi wall nanotube and MWNT are used interchangeably and shall be defined as a coaxial assembly of nanotubes similar to a coaxial cable, or as a molecular sheet, e.g. of graphene, rolled into the shape of a scroll. Examples include multi wall carbon nanotubes, which is used interchangeably with MWCNT.

### Nanotubes

Nanotube shall be defined as a hollow cylindrical or toroidal molecule, which is shorter than 1,000 nanometers in at least one dimension. Examples of nanotubes include single-walled nanotubes (SWNTs), multi-walled nanotube (MWNTs), and boron nitride nanotube (BNNTs);

### Natural

Natural as used herein, refers to entities, which are found abundantly in nature, such as in biological systems. For example, a natural peptide is composed of the twenty natural amino acids; Isoleucine, Alanine, Leucine, Asparagine, Lysine, Aspartic Acid, Methionine, Cysteine, Phenylalanine, Glutamic Acid, Threonine, Glutamine, Tryptophan, Glycine, Valine, Proline, Serine, Tyrosine, Arginine, Histidine. Similarly, a natural oligonucleotide is composed of the four natural nucleotides cytidine, adenosine, guanosine, and thymidine.

### Non-natural

The term, “non-natural” as used herein, refers to entities, which are not found abundantly in nature, such as in biological systems. For example, a non-natural peptide is a peptide, which comprises an entity not found in the list of natural amino acids. Similarly, a non-natural oligonucleotide is an oligonucleotide, which comprises an entity that is not found in the list of the four natural nucleosides.

### Organic

Organic materials or structural entities shall mean materials or entities, which contain carbon and at least one other element.

### Partitioning

The term, “partitioning” as used herein, refers to a process whereby molecules a separated on the basis of one or more of their characteristics. As a non-limiting example, molecules which preferentially bind to a surface or material may be partitioned (separated) from molecules that are not bound to said surface or material.

### Polymer

Polymer shall mean a long, repeating chain, such as a branched chain, of atoms, comprising repeated identical or similar units, formed through the linkage of many monomers. Thus, a polymer is a molecule of high relative molecular mass, the structure of which essentially comprises the multiple repetition of units derived, actually or conceptually, from molecules of low relative molecular mass. The monomers can be identical, or they can have one or more substituted chemical groups. Some polymers are made of more than one kind of monomer. Example polymers are polyvinylchloride, polystyrene, DNA, protein and polypeptide.

### Prosome

The term“prosome” as used herein refers to a complex of a mRNA molecule and two or more ribosomes that is formed during active translation.

Protic solvent

The term, “protic solvent” as used herein, refers to molecular solvent which contains a dissociable proton. The molecules of such solvents can donate a proton. Conversely, aprotic solvents cannot donate protons.

Repeat unit

Repeat unit shall mean the minimal repeated unit of a polymer. For example, the repeat unit of polyethylene ((—CH₂—CH₂—)ₙ) is (—CH₂—CH₂—)

Reactive polymer unit

The term, “reactive polymer unit” as used herein refers to a unit which can polymerize to form a polymer, for example ethylene units (H₂C═CH₂) can react to form polyethylene (—CH₂—CH₂—)ₙ.

Rotatable bond

Rotatable bond shall be defined as a bond that can rotate without breaking one or more chemical bond(s).

Scientific notation

In the present document, scientific notation is used for numbers as follows; [coefficient]E+[exponent], e.g. 100 is written as 1E+2, where 1 is the coefficient and 2 is the exponent (“+” indicates that the exponent is positive).

### Screening/selection

Screening and selection is used interchangeably and shall mean a process or method of identifying one or more molecules with one or more desired characteristics from a library of molecules, e.g. by contacting said library with a material and partitioning a fraction of said library.

Single-walled nanotubes

Single wall nanotube is used interchangeably with SWNT and shall mean a cylindrical nanostructure or a molecular tube with at least one dimension less than 1,000 nanometers. Examples include single wall carbon nanotubes, which is used interchangeable with SWNT.

### Solution

Solution is used interchangeably with dispersion, suspension and colloid, and shall be defined as a liquid medium comprising one or more material(s).

### Solvent

Solvent shall be defined as a component of a medium. Said component may be capable of solvating a molecule, fragment, linker, tag, surface or material.

### Surface

The term, “surface” as used herein, shall mean a material layer constituting a boundary, such as the one or few outermost atomic layer(s) of a material.

### Surfactant

The term, “surfactants” as used herein, refers to compounds that modulate the surface tension of a liquid, allowing easier spreading, modulate the interfacial tension between two liquids, or between a liquid and a solid. Surfactants may act as detergents, wetting agents, emulsifiers, foaming agents, and dispersants.

### Tag

The term, “tag” as used herein, refers to an entity which is linked to a molecule and encodes the molecule or provides other information about said molecules or provides one or more functionalities. A single tag may encode multiple fragments or molecules. Multiple tags may encode a single fragment or molecule. Tags may also have other characteristics such as a high affinity for one or more surfaces or materials.

Thermal conductivity

The term, “thermal conductivity” as used herein, refers to a characteristic of a material describing its ability to conduct heat. Thermal conductivity can be measured in watts per kelvin-metre (W·K-1·m-1, i.e. W/(K-m).

### DETAILED DISCLOSURE OF THE INVENTION

In an embodiment of the invention the method comprises the following steps:

Step A. Providing a library of molecules of the general composition M(n)-L(k)-T(s):

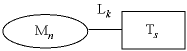

where n=0 to 1E+9, k=0 to 1E+9, and s=0 to 1E+9, such that n+k+s is at least 1, and where M is a library molecule, T is a tag that may be used to identify the library molecule M to which it is attached, and L is a linker that connects M and T;
Step B. Providing a medium;
Step C. Providing a surface or material;
Step D. Combining one or more of said library, medium, surface and/or material;
Step E. Optionally performing one or more manipulations of said library, medium, surface and/or material;
Step F. Partitioning or isolating a fraction of said library;
Step G. Optionally amplifying or copying the whole or a part of one or more molecules;
Step H. Optionally identifying one or more characteristics of one or more library molecules, or isolating one or more library molecules;
wherein
steps A, B, C, D, E, F, G, and H are performed simultaneously or sequentially in any possible order;
and wherein
steps A, B, C, D, E, F, G, and H are performed one or more times;
and wherein
said medium optionally comprises an organic solvent, such as a water-miscible solvent, water or ionic liquids;
said medium optionally comprises a liquid and/or soluble polymer, e.g. caprolactone or short-length nylon;
said surface or material is organic or inorganic, such as for example a fullerene, such as a carbon nanotube or a graphene, a composite material, or a metal;
said partitioning is optionally based on the affinity of one or more library molecules towards a material/surface;
said identification is optionally achieved by sequencing a tag.

Said method may be referred to as a screening and/or a selection. For example, if a partitioning is achieved based on affinity, said method may be referred to as an affinity screening and/or an affinity selection.

In the following the individual steps of the method according to the present invention will be described in more detail.

### Step A. Providing a Suitable Library of Molecules

In step (A) of the present invention a suitable library of molecules is provided. A library is a collection of molecules, comprising at least two molecules. Such a library may comprise few or many molecules, and the molecules may be of any type; for example, but not limited to small organic molecules, peptide, proteins, or polynucleotides. The characteristics of the individual molecules of the library and the total number of molecules of the library strongly influences the probability of identifying molecules with desired characteristics from the library.

It is generally accepted that the greater the size of a library, the greater the probability is that the library comprises molecules with a desired characteristic such as for example a high affinity for a surface or material. Furthermore, it is generally not possibly to deduce, predict or determine e.g. the affinity of a molecule for a surface or material from the structure of said molecule or from its characteristics such as size or atomic composition.

In cases where the molecule characteristics sought (e.g. affinity towards a given surface) are not known to depend on the presence of certain types of chemical moieties in the molecule, such as e.g. amino groups, it may be an advantage to try out many different types of molecules. Therefore, in preferred embodiments, to increase the probability that a molecule library comprises a molecule with a certain characteristic (e.g. a high affinity for a surface or a material), the molecule library should have a high chemical diversity and complexity of molecules and chemical moieties represented.

On the other hand, in cases where it is known that certain chemical moieties or other general characteristics is generally advantageous for the characteristics sought, a low complexity is generally preferred, because it will be advantageous to include many expectably beneficial chemical moieties in the library. As an example, if a molecule is sought that is capable of binding to a negatively charged surface, it may be advantageous to employ a library comprising many molecules with positively charged chemical moieties.

Said molecules may be tagged or untagged, i.e. a tag may be attached to the individual molecule, in order to e.g. allow its easy identification.

Thus, the libraries appropriate for this invention comprise molecules of the general composition M(n)-L(k)-T(s):

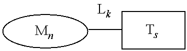

where n=0 to 1E+9, k=0 to 1E+9, and s=0 to 1E+9, such that n+k+s is at least 1, and where M is the (variable) library molecule, T is a tag that may be used to identify the library molecule M to which it is attached, and L is the linker that connects M and T.

Suitable libraries for the present invention is a phage display library, where M is the peptide displayed on the phage, T is the phage particle comprising genetic information which can be sequenced and thus decoded to reveal the structure of the peptide M, and L is the amide bond linking the peptide to the phage particle T.

Other suitable libraries for the present invention include DNA-encoded small molecule libraries such as those described in Rasmussen (2006) WO 06/053571A2, Liu et al. (2002), WO 02/074929 A2; Pedersen et al. (2002) WO 02/103008 A2; Pedersen et al. (2003) WO03/078625 A2; Harbury and Halpin, WO 00/23458, and Hansen et al WO 06/048025.

In these libraries, small molecules (M) are encoded by DNA tags (T) and linked to said tags typically via a PEG (polyethylene glycol) linker. Thus, by sequencing the DNA tag (T) the structure of the linked small molecule M can be deduced. In a preferred embodiment the linker L does not interfere or modify the characteristics of the library molecule M. For example, it is preferred that the linker does not interfere with the ability of the library molecule to bind to a surface/material.

Library molecules do not need to be tagged. Thus, molecules of the general composition M, M-L, L-T and M-L-T are all three appropriate for this invention. Furthermore, many molecules, e.g. such as 100 molecules may be linked to the same tag. Also, one molecule (n=1) may be linked via more than one linker, for example 2 to 10 linkers (e.g. k=2 to 10) to a single tag. Also, more than one tag, for example 10 tags (s=10) may be linked to the same molecule M via one or more linkers.

Preferred Libraries: M, L and T Combinations

Particularly preferred combinations of molecules (M), tags (T), and linkers (L) include the following; combinations where M is a peptide, the linker is a phage, and the tag is a DNA. Examples include M13 phage display libraries, Lampbda phage display libraries, see example T.1.1.1.1.1.1.1.1., Libraries where is a peptide, the linker is a cell, and the tag is a DNA. Examples include E. coli display libraries, see e.g. example 1.1.1.1 and Brown et al., 2008, Small April; 4 (4):416-20). Combinations where M is a small organic molecule, the linker is a relatively short organic molecule (e.g. PEG or C6), and the tag is a DNA. Examples include Chemetics libraries, see for example, Rasmussen (2006) WO 06/053571A2, Liu et al. (2002), WO 02/074929 A2; Pedersen et al. (2002) WO 02/103008 A2; Pedersen et al. (2003) WO03/078625 A2; Harbury and Halpin, WO 00/23458, and Hansen et al WO 06/048025.

Other preferred libraries may be chosen from the following non-limiting list of libraries; Cell display libraries, where M is a peptide or protein optionally modified with one or more organic molecules, L is a cell, such as a mammalian cell or a non-mammalian cell, such as a yeast cell or an insect cell, and T is DNA. Bacterial display, where M is a peptide or protein, optionally modified with one or more organic molecules, L is a bacterial cell, and T is DNA. Virus display, where M is a peptide or protein, optionally modified with one or more organic molecules, L is a virus such as a retrovirus, and T is RNA or DNA. Virus-like particle display, where M is a peptide or protein, optionally modified with one or more organic molecules, L is a virus-like particle, and T is RNA or DNA.

Preferred libraries include; a DNA-encoded small molecule library, a DNA-encoded peptide library, a DNA-encoded macrocycle, a DNA-encoded peptide macrocycle, a DNA-encoded protein, a DNA-encoded DNA library, a DNA-encoded RNA library, a phage-encoded small molecule library, a phage-encoded peptide library, a phage-encoded macrocycle, a phage-encoded peptide macrocycle, a phage-encoded protein, a phage-encoded DNA library, a phage-encoded RNA library, a bacteria-encoded small molecule library, a bacteria-encoded peptide library, a bacteria-encoded macrocycle, a bacteria-encoded peptide macrocycle, a bacteria-encoded protein, a bacteria-encoded DNA library, a bacteria-encoded RNA library, a cell-encoded small molecule library, a cell-encoded peptide library, a cell-encoded macrocycle, a cell-encoded peptide macrocycle, a cell-encoded protein, a cell-encoded DNA library, a cell-encoded RNA library, a mass tag encoded small molecule library, a mass tag encoded peptide library, a mass tag encoded macrocycle, a mass tag encoded peptide macrocycle, a mass tag encoded protein, a mass tag encoded DNA library, a mass tag encoded RNA library, a peptide encoded small molecule library, a peptide encoded peptide library, a peptide encoded macrocycle, a peptide encoded peptide macrocycle, a peptide encoded protein, a peptide encoded DNA library, a peptide encoded RNA library, a RNA-encoded small molecule library, a RNA-encoded peptide library, a RNA-encoded macrocycle, a RNA-encoded peptide macrocycle, a RNA-encoded protein, a RNA-encoded DNA library, a RNA-encoded RNA library.

M, the Library Molecule

It is typically the molecule M that have the characteristics sought when screening a library. In a preferred embodiment of the invention, the library comprises a large number of different molecules M, each of which is attached to a same or very similar linker L, which again is attached to a tag T. Often, all the Ts are of the same kind (e.g. they are all nucleic acids) but of different specific composition (e.g. different sequence of bases in the DNA).

In a preferred embodiment, all of M-L-T is considered the library molecule, and the linker L and/or the tag T intereferes with or modifies the characteristics of the molecule M.

In the present invention, a library of molecules are taken through a process that allows the identification of one or more individual molecules with desired characteristics, from the library of molecules. The degree of success in identifying or isolating molecules with desired characteristics, by using the present invention, depends on the characteristics of the individual library molecules of a given library. Different characteristics of the library molecules are important for different characteristics sought. These characteristics of the individual molecules include molecular weight (MW), different atomic elements, hydrogen bond donors, hydrogen bond acceptors, rotatable bonds, number of atoms, charge, calculated water-octanol partitioning coefficient, polar surface area. Below the relevance of each of these molecule characteristics is described.

In some cases, the degree of success in identifying or isolating molecules with the desired characteristics from a library of molecules, through the use of the present invention, depends on the average characteristics (e.g. the average molecular weight) of the library molecules.

In some cases, the degree of success in identifying or isolating molecules with the desired characteristics from a library of molecules, through the use of the present invention, depends on the diversity of characteristics (e.g. the diversity of functional groups) of the library molecules.

In some cases, the degree of success in identifying or isolating molecules with the desired characteristics from a library of molecules, through the use of the present invention, depends on the number of library molecules that have a certain set of characteristics (e.g. has either an amino group or a carboxylic acid group). Such a set of characteristics may be defined as being below a certain threshold for a given molecule characteristics (e.g. below a molecular weight of 500), and/or above a certain threshold for another molecule characteristics (e.g. comprises more than one pyrene moiety).

The size of a library (i.e. number of molecules of a library) is therefore a very important parameter. In some cases large libraries are advantageous. This is for example the case if the characteristics sought are very challenging (e.g. a very high affinity is sought). In other cases, e.g. when the selection system has little capacity or provides too many false positives when too many library molecules are taken through the enrichment process, it can be advantageous to employ smaller libraries. Also, the costs of generating a library typically increases as its size is increased. To improve the chance of finding molecules with desired characteristics when screening a library it is important to have many different molecules in a library, i.e to have a high diversity. Thus, depending on context, the number of different molecules in a library is preferably more than 10, such as more than 100, such as more than 10², such as more than 10³, such as more than 10⁴, such as more than 10⁵, such as more than 10⁶, such as more than 10⁷, such as more than 10⁸, such as more than 10⁹, such as more than 10¹⁰, such as more than 10¹¹, such as more than 10¹², such as more than 10¹³, such as more than 10¹⁴, such as more than 10¹⁵, such as more than 10¹⁶, such as more than 10¹⁷, such as more than 10¹⁸, such as more than 10¹⁹, such as more than 10²⁰, such as more than 10²¹, such as more than 10²², such as more than 10²³. To reduce cost of producing a library it is important to limit the number of different molecules in the library. Thus, depending on context, the number of different molecules in a library is preferably less than 10²³, such as less than 10²², such as less than 10²¹, such as less than 10²⁰, such as less than 10¹⁹, such as less than 10¹⁸, such as less than 10¹⁷, such as less than 10¹⁶, such as less than 10¹⁵, such as less than 10¹⁴, such as less than 10¹³, such as less than 10¹², such as less than 10¹¹, such as less than 10¹⁰, such as less than 10⁹, such as less than 10⁸, such as less than 10⁷, such as less than 10⁶, such as less than 10⁵, such as less than 10⁴, such as less than 10³, such as less than 10², such as less than 10.

Above it was described how the number of library molecules with a given set of characteristics can affect the degree of success in using the present invention. In some cases it is advantageous if the library comprises a large number of molecules with a certain set of characteristics. This is for example the case if the characteristics sought are very challenging (e.g. a very high affinity is sought), and if it is known that certain chemical functionalities are particularly relevant for the characteristics sought (e.g. it is known that pyrenes can mediate tight binding to the target surface). In these cases, it may be advantageous to use libraries comprising a large number of molecules with a given kind of functionalities, e.g. pyrenes. In other cases, e.g. when the selection system has little capacity or provides too many false positives when too many library molecules comprising a certain chemical moiety (e.g. a thiol) are taken through the enrichment process, it can be advantageous to employ smaller libraries.

In cases where the present invention involves an isolation of e.g. molecules from a library, based on binding to a given surface, the average number of identical copies of each library molecule is important. In some cases a high copy number is advantageous. This is for example the case when many ligands to a surface is sought. The high copy number will allow a higher number of library molecules to be isolated in a selection for affinity to a target. On the other hand, if only one or a few ligands to a surface are sought, it may be advantageous to use libraries with lower copy numbers. Also, to reduce the risk that molecules are stochastically lost during library handling, including partitioning steps, it is important to have a sufficiently high number of each molecule present in the library. For example, if many partitioning steps, such as more than 3 steps, are performed in series, more copies of each molecule is needed as compared to when fewer partitioning steps, such as less than 4 steps, are performed in series, because a fraction of the library will be lost due to handling, sticking to surfaces etc. Thus depending on context, the number of each molecule present in a library is preferably more than 10, such as more than 100, such as more than 10², such as more than 10³, such as more than 10⁴, such as more than 10⁵, such as more than 10⁶, such as more than 10⁷, such as more than 10⁸, such as more than 10⁹, such as more than 10¹⁰, such as more than 10¹¹, such as more than 10¹², such as more than 10¹³, such as more than 10¹⁴, such as more than 10¹⁵. To reduce cost of producing a library it is important to limit the number of each molecule in the library. Thus, depending on context, the number of each molecule in a library is preferably less than 10¹⁵, such as less than 10¹⁴, such as less than 10¹³, such as less than 10¹², such as less than 10¹¹, such as less than 10¹⁰, such as less than 10⁹, such as less than 10⁸, such as less than 10⁷, such as less than 10⁶, such as less than 10⁵, such as less than 10⁴, such as less than 10³, such as less than 10², such as less than 10, such as 1. The number of each molecule in a library may also be similar or significantly different. For example, when a library is first synthesized it is preferable that the number of each molecule is essentially similar such that an overreåresentation of certain library molecules in the library is avoided. After performing one or more partitioning steps, it is expected that those molecules which most efficiently bind and are released will be present in the library in a higher number than molecules which do not efficiently bind and become released.

The M moiety may contain one, two or more independent or semi-independent moieties, held together by a linker. This situation is shown below for the case where M is made up of two moieties, M1 and M2.

Scheme with M1 and M2 linked to a tag in parallel:

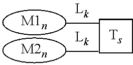

Scheme with M1 and M2 linked to a tag in series:

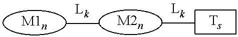

Scheme with M displayed from a linker L which is a cell or a virus, which contains a tag T

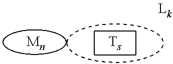

M1 and M2 may be identical or different. Also, M1 or M2 may represent a number of different molecules of the library, whereas the other is kept fixed, i.e. is the same for all library members. M1 can be used as an anchor moiety, i.e. a moiety that helps M2 bind with higher total affinity to the target surface. This may be advantageous in cases where M2 molecules are sought that bind to a surface, but where the library members do not in themselves represent a high enough affinity, e.g. because the conditions under the isolation process are very stringent and do not retain the M2 moiety unless its binding is improved by being linked to an anchor moiety with some affinity for the target surface. M2 can of course also be used as the anchor moiety and M1 represent the library members. Sometimes a ligand for a surface or material is known, and a linker that can connect two ligands and thereby improve binding, is sought. The linker can then be optimized, by employing fixed M1 and M2 moieties for all library members (i.e. all library members have identical M1 and M2 moieties. Then the linker is varied, and thus represents the library members, i.e. different M moieties of a library will differ only in their linker moiety.

Preferred Library Molecules

In preferred embodiments, molecules are peptides. For example, peptides which bind to graphene may be used to assemble or construct advanced materials that are biodegradable because the peptides are biodegradable. In other preferred embodiments molecules comprise polynucleotides such as gold-binding polynucleotides which may be used to assemble gold structures using polynucleotide recognition.

In preferred embodiments, molecules are chosen from

Organic molecules, such as amino acids such as, L-amino acids, D-amino acids, alpha amino acids, beta amino acids, gamma amino acids, essential amino acids, non-essential amino acids, imino acids, N-substituted L-amino acids, N-substituted D-amino acids, N-substituted alpha amino acids, N-substituted beta amino acids, N-substituted gamma amino acids, N-substituted essential amino acids, N-substituted non-essential amino acids, N-substituted imino acids,
peptides, such as, Peptides comprising L-amino acids, Peptides comprising D-amino acids, Peptides comprising alpha amino acids, Peptides comprising beta amino acids, Peptides comprising gamma amino acids, Peptides comprising essential amino acids, Peptides comprising non-essential amino acids, Peptides comprising imino acids, Peptides comprising N-substituted L-amino acids, Peptides comprising N-substituted D-amino acids, Peptides comprising N-substituted alpha amino acids, Peptides comprising N-substituted beta amino acids, Peptides comprising N-substituted gamma amino acids, Peptides comprising N-substituted essential amino acids, Peptides comprising N-substituted non-essential amino acids, Peptides comprising N-substituted imino acids,
Proteins, such as antibodies, antibody fragments, such as, VhH domains, V-NAR domain, VH domains, VL domains, Camel Ig, IgNAR, IgG, Fab, Fab2, Fab3, Bis-scFv, Minibody (bivalent), scFV, Triabody, Diabody, Tetrabody, Enzymes, Carbohydrates, such as, linear carbohydrates, branched carbohydrates, monosaccharides, disaccharides, oligosaccharides, polysaccharides, Lipids, such as, Sterols, fatty acids, waxes, monoglycerides, diglycerides, phospholipids, fatty acyls, glycerolipids, glycerophospholipids, sphingolipids, saccharolipids, polyketides, Nucleic acids, such as, RNA, mRNA, rRNA, tRNA, tmRNA, snRNA, snoRNA, scaRNA, gRNA, aRNA, crRNA, ncRNA, miRNA, piRNA, siRNA, tasiRNA, rasiRNA, PNA, LNA, DNA, Mixed organic/inorganic molecules.

In preferred embodiments, molecules are chosen from small compact molecules, linear organic molecules, polymers, polypeptides, poly-ureas, polycarbamates, scaffold structures, cyclic structures, natural compound derivatives, alpha-, beta-, gamma-, and omega-peptides, mono-, di- and tri-substituted peptides, L- and D-form peptides, cyclohexane- and cydopentane-backbone modified beta-peptides, vinylogous polypeptides, glycopolypeptides, polyamides, vinylogous sulfonamide peptide, Polysulfonamide conjugated peptide (i.e., having prosthetic groups), Polyesters, Polysaccharides, polycarbamates, polycarbonates, polyureas, poly-peptidylphosphonates, Azatïdes, peptoids (oligo N-substituted glycines), Polyethers, ethoxyformacetal oligomers, poly-thioethers, polyethylene, glycols (PEG), polyethylenes, polydisvlfides, polyarylene sulfides, polynucleotides, PNAs, LNAs, Morpholinos, oligo pyrrolinone, polyoximes, Polyimines, Polyethyleieimine, Polyacetates, Polystyrenes, Polyacetylene, Polyvinyl, Lipids, Phospholipids, Glycolipids, poiycycles, (aliphatic), polycycles (aromatic), polyheterocydes, Proteoglycan, Polysiloxanes, Polyisocyanides, Polyisocyanates, polymethacryiates, Monofunctional, Difunctional, Trifunctional and Oligofunctional open-chain hydrocarbons. Monofunctional, Difunctional, Trifunctional and Oligofunctional Nonaromat Carbocycles, Monocyclic, Bicydic, Tricyclic and Polycydic Hydrocarbons, Bridged Polycyclic Hydrocarbones, Monofunctional, Difunctional, Trifunctional and Oligofunctional Nonaromatic, Heterocycles, Monocyclic, Bicydic, Tricyclic and Polycyclic Heterocycles, bridged Polycyclic Heterocycles, Monofunctional, Difunctional, Trifunctional and Oligofunctional Aromatic Carbocycles. Monocyclic, Bicydic, Tricyclic and Polycyclic Aromatic Carbocycles, Monofunctional, Difunctional, Trifunctional and Oligofunctional Aromatic Hetero-cycles. Monocyclic, Bicydic, Tricyclic and Polycyclic Heterocycles. Chelates, fullerenes, and any combination of the above and many others.

### Peptides

In preferred embodiments, molecules are peptides composed of only natural amino acids, i.e.; Isoleucine, Alanine, Leucine, Asparagine, Lysine, Aspartic Acid, Methionine, Cysteine, Phenylalanine, Glutamic Acid, Threonine, Glutamine, Tryptophan, Glycine, Valine, Proline, Serine, Tyrosine, Arginine, or Histidine. In other preferred embodiments, the molecule is a peptide comprising a non-natural amino acid, a beta amino acid, a gamma amino acid, or a non-natural zwitter ion. In other embodiments, the molecule is a peptide comprising only alpha amino acids. In other embodiments, the molecule is a peptide comprising at least 1 alpha amino acid.

### Fragments/Moieties

In preferred embodiments, library molecules comprise fragments which are connected by chemical bonds and/or linking groups formed by a chemical reaction. For example, peptides comprise fragments, i.e. amino acids, which are connected by linking groups, i.e. amide bonds, formed by chemical reactions. As another example, DNA library molecules comprise fragments, i.e., nucleotides, which are linked by groups, i.e. phosphodiesters, formed by a chemical reaction.

Preferred fragments are chosen from the following non-limiting list; amino acids, nucleotides, C3-C10 cycloalkyl, aryl, heterocyclyl, heteroaryl, aryl-d-C6 alkyl, C3-C10 cycloalkyl-aryl, aryl-C3-C10 cycloalkyl, C3-C10 cycloalkyl-heterocyclyl, heterocyclyl-C3-C10 cycloalkyl, C3-C10 cycloalkyl-heteroaryl, heteroaryl-C3-C10 cycloalkyl, aryl-heterocyclyl, heterocyclyl-aryl, aryl-heteroaryl, heteroaryl-aryl, heterocyclyl-heteroaryl, heteroaryl-heterocyclyl, C3-C10 cycloalkyl-O-aryl, aryl-O—C3-C10 cycloalkyl, C3-C10 cycloalkyl-O-heterocyclyl, heterocyclyl-O—C3-C10 cycloalkyl, C3-C10 cycloalkyl-O-heteroaryl, heteroaryl-O—C3-C10 cycloalkyl, aryl-O-heterocyclyl, heterocyclyl-O-aryl, aryl-O-heteroaryl, heteroaryl-O-aryl, heterocyclyl-O-heteroaryl, heteroaryl-O-heterocyclyl, C3-C10 cycloalkyl-C(O)-aryl, aryl-C(O)—C3-C10 cycloalkyl, C3-C10 cycloalkyl-C(O)-heterocyclyl, heterocyclyl-C(O)—C3-C10 cycloalkyl, C3-C10 cycloalkyl-C(O)-heteroaryl, heteroaryl-C(O)—C3-C10 cycloalkyl, aryl-C(O)-heterocyclyl, heterocyclyl-C(O)-aryl, aryl-C(O)-heteroaryl, heteroaryl-C(O)-aryl, heterocyclyl-C(O)-heteroaryl, heteroaryl-C(O)-heterocyclyl, C3-C10 cycloalkyl-CHC2aryl, aryl-CH2-C3-C10 cycloalkyl, C3-C10 cycloalkyl-CH2-heterocyclyl, heterocyclyl-CH2-C3-C10 cycloalkyl, C3-C10 cycloalkyl-CHC2heteroaryl, heteroaryl-CH2-C3-C10 cycloalkyl, aryl-CH2-heterocyclyl, heterocyclyl-CHC2aryl, aryl-CH2-heteroaryl, heteroaryl-CH2-aryl, heterocyclyl-CHC2heteroaryl, heteroaryl-CH2-heterocyclyl, C3-C10 cycloalkyl-CH2CH2-aryl, aryl-CH2CH2-C3-C10 cycloalkyl, C3-C10 cycloalkyl-CH2CH2-heterocyclyl, heterocyclyl-CH2CH2-C3-C10 cycloalkyl, C3-C10 cycloalkyl-CH2CH2-heteroaryl, heteroaryl-CH2CH2-C3-C10 cycloalkyl, aryl-CH2CH2-heterocyclyl, heterocyclyl-CH2CH2-aryl, aryl-CH2CH2-heteroaryl, heteroaryl-CH2CH2-aryl, heterocyclyl-CH2CH2-heteroaryl, heteroaryl-CH2CH2-heterocyclyl, C3-C10 cycloalkyl-NH-aryl, aryl-NH—C3-C10 cycloalkyl, C3-C10 cycloalkyl-NH-heterocyclyl, heterocyclyl-NH—C5-C10 cycloalkyl, C3-C10 cycloalkyl-NH-heteroaryl, heteroaryl-NH—C3-C10 cycloalkyl, aryl-NH-heterocyclyl, heterocyclyl-NH-aryl, aryl-NH-heteroaryl, heteroaryl-NH-aryl, heterocyclyl-NH-heteroaryl, heteroaryl-NH-heterocyclyl, C3-C10 cycloalkyl-N(Me)-aryl, aryl-N(Me)-C3-C10 cycloalkyl, C3-C10 cycloalkyl-N(Me)-heterocyclyl, heterocyclyl-N(Me)-C3-C10 cycloalkyl, C3-C10 cycloalkyl-N(Me)-heteroaryl, heteroaryl-N(Me)-C3-C10 cycloalkyl, aryl-N(Me)-heterocyclyl, heterocyclyl-N(Me)-aryl, aryl-N(Me)-heteroaryl, heteroaryl-N(Me)-aryl, heterocyclyl-N(Me)-heteroaryl, heteroaryl-N(Me)-heterocyclyl, C3-C10 cycloalkyl-NHC(O)-aryl, aryl-NHC(O)—C3-C10 cycloalkyl, C3-C10 cycloalkyl-NHC(O)-heterocyclyl, heterocyclyl-NHC(O)—C3-C10 cycloalkyl, C3-C10 cycloalkyl-NHC(O)-heteroaryl, heteroaryl-NHC(O)—C3-C10 cycloalkyl, aryl-NHC(O)-heterocyclyl, heterocyclyl-NHC(O)-aryl, aryl-NHC(O)-heteroaryl, heteroaryl-NHC(O)-aryl, heterocyclyl-NHC(O)-heteroaryl, heteroaryl-NHC(O)-heterocyclyl, C3-C10 cycloalkyl-C(O)NH-aryl, aryl-C(O)NH—C3-C10 cycloalkyl, C3-C10 cycloalkyl-C(O)NH-heterocyclyl, heterocyclyl-C(O)NH—C3-C10 cycloalkyl, C3-C10 cycloalkyl-C(O)NH-heteroaryl, heteroaryl-C(O)NH—C3-C10 cycloalkyl, aryl-C(O)NH-heterocyclyl, heterocyclyl-C(O)NH-aryl, aryl-C(O)NH-heteroaryl, heteroaryl-C(O)NH-aryl, heterocyclyl-C(O)NH-heteroaryl, heteroaryl-C(O)NH-heterocyclyl, C3-C10 cycloalkyl-NHC(O)NH-aryl, aryl-NHC(O)NH—C3-C10 cycloalkyl, C3-C10 cycloalkyl-NHC(O)NH-heterocyclyl, heterocyclyl-NHC(O)NH—C3-C10 cycloalkyl, C3-C10 cycloalkyl-NHC(O)NH-heteroaryl, heteroaryl-NHC(O)NH—C3-C10 cycloalkyl, aryl-NHC(O)NH-heterocyclyl, heterocyclyl-NHC(O)NH-aryl, aryl-NHC(O)NH-heteroaryl, heteroaryl-NHC(O)NH-aryl, heterocyclyl-NHC(O)NH-heteroaryl, and heteroaryl-NHC(O)NH-heterocyclyl; wherein any alkyl, cycloalkyl, aryl, heterocyclyl, and heteroaryl optionally may be substituted.

Further preferred fragments include

[p]Annulene, Aceanthrylene, Acenaphthylene, Acephenanthrylene, Anthracene, as-Indacene, Azulene, Benzene, Biphenylene, Chrysene, Coronene, Decacene, Decahelicene, Decaphene, Fluoranthene, Fluorene, Heptacene, Heptahelicene, Heptaphene, Heptaphenylene, Hexacene, Hexahelicene, Hexaphene, Hexaphenylene, Indene, Naphthalene, Nonacene, Nonahelicene, Nonaphene, Nonaphenylene, Octacene, Octahelicene, Octaphene, Octaphenylene, Ovalene, Pentacene, Pentalene, Pentaphene, Pentaphenylene, Perylene, Phenalene, Phenanthrene, Picene, Pleiadene, Polyacene, Polyalene, Polyalene, Polyaphene, Polyhelicene, Polynaphthylene, Polyphenylene, Pyranthrene, Pyrene, Rubicene, s-Indacene, Tetracene, Tetranaphthylene, Tetraphene, Tetraphenylene, Trinaphthylene, Triphenylene, each optionally substituted.

Yet further preferred fragments include

1,10-Phenanthroline, 1,5-Naphthyridine, 1,6-Naphthyridine, 1,7-Naphthyridine, 1,7-Phenanthroline, 1,8-Naphthyridine, 1,8-Phenanthroline, 1,9-Phenanthroline, 2,6-Naphthyridine, 2,7-Naphthyridine, 2,7-Phenanthroline, 2,8-Phenanthroline, 2,9-Phenanthroline, 3,7-Phenanthroline, 3,8-Phenanthroline, 4,7-Phenanthroline, Acridarsine, Acridine, Acridophosphine, Arsanthrene, Arsanthridine, Arsindole, Arsindolizine, Arsinoline, Arsinolizine, Boranthrene, Carbazole, Chromene, Cinnoline, Furan, Imidazole, Indazole, Indole, Indolizine, Isoarsindole, Isoarsinoline, Isochromene, Isoindole, Isophosphindole, Isophosphinoline, Isoquinoline, Isoselenochromene, Isotellurochromene, Isothiochromene, Mercuranthrene, Oxanthrene, Perimidine, Phenanthridine, Phenarsazinine, Phenazine, Phenomercurazine, Phenophosphazinine, Phenoselenazine, Phenotellurazine, Phenothiarsinine, Phenothiazine, Phenoxaphosphinine, Phenoxarsinine, Phenoxaselenine, Phenoxastibinine, Phenoxatellurine, Phenoxathiine, Phenoxazine, Phosphanthrene, Phosphanthridine, Phosphindole, Phosphindolizine, Phosphinoline, Phosphinolizine, Phthalazine, Pteridine, Purine, Pyran, Pyrazine, Pyrazole, Pyridazine, Pyridine, Pyrimidine, Pyrrole, Pyrrolizine, Quinazoline, Quinoline, Quinolizine, Quinoxaline, Selenanthrene, Selenochromene, Selenoxanthene, Silanthrene, Telluranthrene, Tellurochromene, Telluroxanthene, Thianthrene, Thiochromene, Thioxanthene, Xanthene.

### Chemical Bonds or Chemical Entities Formed by a Chemical Reaction

A library molecule is typically composed of fragments linked by a chemical bond or chemical linking group. In preferred embodiments, a chemical bond or a chemical entity linking fragments may be chosen from; A single bond, such as a single carbon-carbon bond, a carbon-heteroatom single bond, a heteroatom-heteroatom single bond, a double bond, such as a carbon-carbon double bond or a carbon-heteroatom double bond, a heteroatom-heteroatom double, bond, a triple bond, such as a carbon-carbon triple bond or a carbon-heteroatom triple bond, a heteroatom-heteroatom triple bond, —CH2-, —C(O)—, —NH—, —O—, —S—, —SO2-, —CH2CH2-, —C(O)CH2-, —CH2C(O)—, —NHCH2-, —CH2NH—, —OCH2-, —CH2O—, —SCH2-, —CH2S—, —SO2CH2-, —CH2SO2-, —NHC(O)—, —C(O)NH—, —NHSO2-, —SO2NH—, —CH2CH2CH2-, —CH2CH2C(O)—, —CH2CH2NH—, —CH2CH2O—, —CH2CH2S—, —CH2CH2SO2-, —CH2C(O)CH2-, —CH2NHCH2-, —CH2OCH2-, —CH2SCH2-, —CH2SO2CH2-, —C(O)CH2CH2-, —NHCH2CH2-, -OCH2CH2-, -SCH2CH2-, -SO2CH2CH2-, —CH2C(O)NH—, —CH2SO2NH—, —CH2NHC(O)—, —CH2NHSO2-, —C(O)NHCH2-, -SO2NHCH2-, —NHC(O)CH2-, -NHSO2CH2-, and —NHC(O)NH—

In preferred embodiments, molecules are chosen from the following list where the plus sign denotes a covalent bond;

Organic molecule+Organic molecule, Organic molecule+Organic macromolecule, Organic molecule+Large organic molecule, Organic molecule+Small organic molecule, Organic molecule+Organic-molecule-binding molecule, Organic molecule+Organic molecule mimic, Organic molecule+Biological molecule, Organic molecule+Biological macromolecule, Organic molecule+Large biomolecule, Organic molecule+Small biomolecule, Organic molecule+Natural biomolecule, Organic molecule+Unnatural biomolecule, Organic molecule+biomolecule-binding molecule, Organic molecule+Biomolecule mimic, Organic molecule+Protein, Organic molecule+Natural protein, Organic molecule+Modified protein, Organic molecule+Unnatural protein, Organic molecule+Protein-binding molecule, Organic molecule+Protein mimic, Organic molecule+Peptide, Organic molecule+Natural peptide, Organic molecule+Modified peptide, Organic molecule+Unnatural peptide, Organic molecule+Peptide-binding molecule, Organic molecule+Peptide mimic, Organic molecule+Nucleic acids, Organic molecule+DNA, Organic molecule+Natural DNA, Organic molecule+Modified DNA, Organic molecule+Unnatural DNA, Organic molecule+DNA-binding molecule, Organic molecule+DNA mimic, Organic molecule+RNA, Organic molecule+Natural RNA, Organic molecule+Modified RNA, Organic molecule+Unnatural RNA, Organic molecule+RNA-binding molecule, Organic molecule+RNA mimic, Organic molecule+snRNA, Organic molecule+Natural snRNA, Organic molecule+Modified snRNA, Organic molecule+Unnatural snRNA, Organic molecule+snRNA-binding molecule, Organic molecule+snRNA mimic, Organic molecule+miRNA, Organic molecule+Natural miRNA, Organic molecule+Modified miRNA, Organic molecule+Unnatural miRNA, Organic molecule+miRNA-binding molecule, Organic molecule+miRNA mimic, Organic molecule+Inorganic molecule, Organic molecule+Inorganic macromolecule, Organic molecule+Large inorganic molecule, Organic molecule+Small inorganic molecule, Organic molecule+Inorganic-molecule-binding molecule, Organic molecule+Inorganic molecule mimic, Organic molecule+Surface-binding molecule, Organic molecule+Material-binding molecule. In preferred embodiments, the molecule is a polymer, such as a polymer composed of only natural amino acids, or a polymer comprising a non-natural amino acid, a polymer comprising a beta amino acid, a polymer comprising a gamma amino acid, a polymer composed of only alpha amino acids, only beta amino acids, only gamma amino acids.

### Molecule Salts

Molecules of the present invention may exist as salts such that any ionic group or moiety of a molecule may be associated with a counterion. For example, a carboxylic acid in ionized form may be associated with a sodium ion (Na+) or another cation such as for example, but not limited to potassium, lithium etc.

### Non-Limiting Examples of Suitable Library Molecules

In preferred embodiments, molecules may be chosen from commercially available collections. Non-limiting examples of suitable collections are sold by Chembridge (San Diego), e.g. EXPRESS-Pick™ Collection; a small molecule screening library collection of 450,000 quality verified, druglike, diverse, small molecule compounds, available for custom selection. These compounds are sourced by ChemBridge through collaborations and researchers and are readily available from stock in mg or micromol amounts. Another non-limiting example, is DIVERSet™—A “universally” diverse collection of 50,000 drug-like small molecules. The chemical library is rationally selected based on 3D pharmacophore analysis to cover the broadest part of biologically relevant pharmacophore diversity space. Another non-limiting example of molecules suitable for the present invention is MicroFormats™—A ready to screen collection of 100,000 to 200,000 small molecules, pre-plated in DMSO in 0.25 umol and higher amounts and 0.1 mg to 5 mg amounts. Another example of molecules suitable for the present invention is MW Set (Molecular Weight Set)—A collection of 30,000 compounds plated in sequential order of increasing molecular weight and can be ordered within particular molecular weight ranges. The molecules are selected for characteristics such as diversity, low molecular weight (200-450), and lower polar surface area, rotatable bond, hydrogen donor, and hydrogen acceptor value ranges. Another example of molecules suitable for the present invention is CNS-Set™—This CNS Library is a collection of 56,000 druglike, small molecule compounds, selected with medicinal chemistry expertise. Computational analysis of CNS-Set includes Polar Surface Area, Lipinski's Rule of 5, and other desirability and drug-like filters, which increase probability of finding leads with oral bio-availability and blood-brain barrier penetration.

Another example of molecules suitable for the present invention is KINASet—This kinase library is a computationally selected collection of 11,000 compounds utilizing a ligand-based pharmacophore selection method. In a preferred embodiment, a molecule or collection of molecules is chosen from the ChemBridge Master Database.

In another preferred embodiment, a molecule or collection of molecules is chosen from molecules registered in the Chemical Abstract Services databases; REGISTRY and/or MARPAT and/or the Beilstein/Reaxys database, ChEMBL, ChemSpider, PubChem as of the date of filing of this patent application.

In preferred embodiments, molecules suitable for the present invention are synthesized by combining two or more fragments derived from any molecule in a collection mentioned above. In preferred embodiments, molecules mentioned above are substituted with one or more chemical entities chosen from; —OH, —NH2, —COOH, —CHO.

### Inorganic Entities

In preferred embodiments, a library molecule and/or a material which is used for screening (to identify ligands with affinity to said material, comprises an inorganic entity chosen from the following list (in chemical notation); AcCl3, AcF3, Ac2O3, AlH3, AlBr, AlBr3, AlCl3, AlnCl(3n−m)(OH)m, AlF3, AlGaAs2, AlGaN2, Al(OH)3, AlI, AlI3, Al(i-PrO)3, Al(NO3)3, AlN, Al2O3, Al2(SO4)3, Al2S3, i-Bu2AlH (DIBAL-H), Et2AlCl, LiAlH4, (—Al(CH3)O-)n, Et3Al, Me3Al, AmCl2, AmCl3, AmF3, AmO2, SbF3, SbF5, SbCl3, SbCl5, Sb2O3, Sb2O5, Sb2S3, Sb2S5, InSb, C8H4K2O12Sb2, KSbF6, NaSbF6, SbH3, (C6H5)3Sb, HArF, AsBr3, AsCl3, AsF5, As2O3, As2O5, As2S3, AsH3, (CH3)2AsOOH, GaAs, KAsF6, KH2AsO4, Na2HAsO4, NaAsO2, (C6H5)3As (CH3)4As2, Ba(OCOCH3)2, BaBr2, BaCO3, BaCl2, BaCuOx, Ba(OH)2, BaF2, BaI2, Ba(NO3)2, BaO, BaO2, BaSO4, BaS, BaTiO3, YBa2Cu3O7-δ or YBCO, BkBr3, Bk2(CO3)3, BkCl3, BkF3, BkI3, Bk(NO3)3, Bk2O3, BkPO4, Bk2(SO4)3, Bk2S3, BeBr2, BeCO3, BeCl2, BeF2, BeH2, Be(OH)2, BeI2, Be(NO3)2, Be3N2, BeO, BeSO4, BeSO3, Be(BH4)2, BeTe, Bi(OCOCH3)3, BiCl3, BiF3, Bi(NO3)3.5H2O, Bi2O3, Bi2Te3 BiOCl, Bi2Sr2CaCu2O8+x, (BiO)2CO3, Bi5O(OH)9(NO3)4, HOC6H4COOBiO, Bi(OSO2CF3)3, (C6H5)3Bi, NH4BF4, C5H5B, BH3.THF, Na2B4O7.10H2O, B3N3H6, H3BO3, B4C, BN, B2O3, B6O, BBr3, BCl3, BF3, H2C2B10H10, B10H14, B2H6, LaB6, Li2B4O7, LiBH4, Li(sec-C4H9)3BH, LiBH(C2H5)3, NaBH4, NaBH3CN, NaBF4, NaB(C6H5)4, NaBH(OCOCH3)3, B4H10, HBF4, TiB2, B(OCH3)3, (CH3BO)3, WB, AlBr3, NH4Br, BBr3, HBrO3, BrCl, Br2O, BrF5</>, BrF3, CaBr2 CBr4, CuBr, CuBr2, HBr(aq), HBr(g), HOBr, IBr, FeBr2, FeBr3, PbBr2, LiBr, MgBr2, Hg2Br2, HgBr2, NOBr, PBr5, PBr3, KBr, KBrO3, KBrO4, SiBr4, AgBr, NaBr, NaBrO3, NaBrO4, SOBr2, SnBr2, ZnBr2, Cd3As2, CdBr2, CdCO3, CdF2, CdCl2, CdI2, Cd(OH)2, CdO, CdSe, CdSO4, CdS, CdTe, Cs2CO3, CsCl, CsF, CsOH, CsI, Cs2SO4, Cs2O, Ca(OCOCH3)2, Ca(HCO3)2, CaB6, CaBr2, CaC2, CaCO3, CaCl2, Calcium citrate, CaNCN, CaF2, CaPO3F, Ca(HOCH2(CHOH)4CO2)2, CaH2, CaHPO4, Ca(OH)2, Ca(OCl)2, CaI2, CaMoO4, Ca(NO3)2, CaC2O4, CaO, CaO2, Ca3(PO4)2, CaSiO3, CaSO4, CaS, CaTiO3, CaWO4, CaZrO3, CfBr3, Cf2(CO3)3, CfCl3, CfF3, CfI3, Cf(NO3)3, Cf2O3, CfPO4, Cf2(SO4)3, Cf2S3, NH4HCO3, NH2COONH4, (NH4)2CO3, NH4SCN, BaCO3, B4C, CdCO3, Cs2CO3, Ca(HCO3)3, CaC2 CaCO3, CaNCN, CO2, CS2, H2CO3, CO, β-C3N4, C3O2, CBr4, CCl4, CF4, COF2, COS, H2C2B10H10, Ce2(CO3)3, Cr(CO)6, CoCO3, Co2(CO)8, Co(SCN)2, CuCO3, CuCN, (HCNO)x NH2CN HCNO, (CN)2, BrCN, CICN, HCN, FeCO3, Fe(CO)5, PbCO3, La2(CO3)3, Li2CO3, MgCO3, MnCO3, Mn2(CO)10, Mo(CO)6, NiCO3, Ni(CO)4, COCl2, KHCO3, K2CO3, KOCN, KCN, K3Fe(CN)6, K4Fe(CN)6, KSCN, Fe4(Fe(CN)6)3, SiC, Ag2CO3, AgCN, NaHCO3, Na2CO3, NaOCN, NaCN, Na3Fe(CN)5NO, NaSCN, SrCO3, TaC, (SCN)2, CSCl2, CS(NH2)2, TiC, WC, W(CO)6, CO(NH2)2, ZnCO3, (NH4)2Ce(NO3)6 (CAN), (NH4)2Ce(SO4)3, Ce2(CO3)3, CeCl3, CeF3, CeO2, CePO4, Ce(SO4)2, Ce(OSO2CF3)3, Ce(OSO2CF3)4, AlCl3, AmCl3, NH4Cl, SbCl3, SbCl5, AsCl3, BaCl2, BeCl2, BiCl3, BCl3, BrCl, CdCl2, CdCl5, CsCl, CaCl2, Ca(OCl)2, CCl4, CeCl3, NH2Cl, ClO2, ClO3, ClF3, H2PtCl6, ClSO3H, ClSO2NCO, CrCl2, CrCl3, CrO2Cl2, CoCl2, CuCl, CuCl2, CmCl3, ClCN, Cl2O7, Cl2O, S2Cl2 DyCl3, ErCl3, EuCl2, EuCl3, GdCl3, GaCl3, GeCl2, GeCl4, AuCl, AuCl3, HfCl4, H2PtCl6, HoCl3, HCl, HCl(aq), HOCl, InCl, InCl3, ICl, IrCl3, FeCl2, FeCl3, LaCl3, PbCl2, LiCl, LiClO4, LuCl3, MgCl2, Mg(ClO4)2, MnCl2, Hg2Cl2, HgCl2, Hg(ClO4)2, MoCl3, MoCl5, NdCl3, NpCl4, NiCl2, NbCl4, NbCl5, NbOCl3, NCl3, NOCl, NO2Cl, OsCl3, PdCl2, COCl2, (PNCl)3, POCl3, PCl5, PCl3, PtCl2, PtCl4, PuCl3, KCl, KClO3, KClO4, PrCl3, PaCl5, RaCl2, ReCl3, ReCl5, RhCl3, RbCl, RuCl3, SmCl3, ScCl3, SeCl2, SeCl4, SiCl4, AgCl, AgClO4, NaClO3, NaClO2, NaCl, NaOCl, NaClO4, SrCl2, SCl2, SO2Cl2, TaCl3, TaCl4, TaCl5, TeCl4, TbCl3, TICl, TICl3, SOCl2, CSCl2, ThCl4, TmCl3, SnCl2, SnCl4, TiCl3, TiCl4, HSiCl3, WCl4, WCl5, WCl6, UCl3, UCl4, UCl5, UCl6, UO2Cl2, VCl2, VCl3, VCl4, VOCl3, [IrCl(CO)(PPh3)2], (Ph3P)3RhCl, YbCl3, YCl3, ZnCl2, ZrCl4, (NH4)2Cr2O7, NH4[Cr(NCS)4(NH3)2], BaCrO4, Cr(C6H6)2, Cr2(CH3COO)4, Cr(acac)3, Cr(CO)6, CrCl2, CrCl4, CrCl3, Cr(NO3)3, Cr2O3, NH4[Cr(SCN)4(NH3)2] CrO2, CrO3, Cr(NO3)3, KCr(SO4)2, Cr2(SO4)3, Cr2S3, CrN, CrO2Cl2, Cu2Cr2O5, PbCrO4, K2CrO4, K2Cr2O7, Na2CrO4, Na2Cr2O7, Co(acac)2, CoBr2, CoCO3, CoCl2, CoF2, CoF3, Co(OH)2, Co(NO3)2, CoO, Co3O4, CoPc, CoSO4, CoS, Co(SCN)2, Cp2Co, Co2(CO)8, Hexol, Na3(Co(NO2)6, Co4(CO)12, Vitamin B12, CuCO3-Cu(OH)2, Cu(OAc)2, CuBr, CuBr2, CuCO3, CuCl, CuCl2, Cu2Cr2O5, CuCN, CuF2, Cu(OH)2, CuI, Cu(NO3)2, Cu2O, CuO, CuSO4, Cu2S, CuS (Cu2S2), R2CuLi, Li2CuCl4, YBCO, CmCl3, Cm2O3, CmO2, Dy2(CO3)3, DyCl3, DyF3, Dy(NO3)3, Dy2O3, DyPO4, Dy2(SO4)3, Dy(OSO2CF3)3, Er2(CO3)3, ErCl3, ErF3, Er(NO3)3, Er2O3, ErPO4, Er2(SO4)3, EsBr3, Es2(CO3)3, EsCl3, EsF3, EsI3, Es(NO3)3, Es2O3, EsPO4, Es2(SO4)3, Es2S3, Eu2(CO3)3, EuCl2, EuCl3, EuF3, Eu(fod)3, Eu(hfc)3, EuI2, Eu(NO3)3, Eu2O3, EuSO4, EuPO4, Eu2(SO4)3, Eu(tmhd)3, Eu(NTf2)3, FmBr3, Fm2(CO3)3, FmCl3, FmF3, FmI3, Fm(NO3)3, Fm2O3, FmPO4, Fm2(SO4)3, Fm2S3, AlF3, AmF3, NH4F, NH4HF2, NH4BF4, SbF5, SbF3, AsF5, AsF3, BaF2, BeF2, BiF3, F5SOOSF5 BF3, BrF, BrF5, BrF3, CdF2, CsF, CaF2, CF4, COF2, CeF3, CeF4, ClF, ClF5, ClF3, CrF3, CrF5, CrO2F2, CoF2, CoF3, CuF2, CmF3, N2F2, N2F4, O2F2, P2F4 S2F2, DyF3, ErF3, EuF3, HBF4, FN3, FOSO2F, FONO2, FSO3H, GdF3, GaF3, GeF4, AuF3, HfF4, H2SbF6, HPF6, H2SiF6, H2TiF6, HF, HF(aq), HOF, InF3, IF7, IF, IF5, IrF3, IrF6, FeF2, FeF3, KrF2, LaF3, PbF2, PbF4, LiF, MgF2, MnF2, MnF3, Hg2F2, HgF2, MoF3, MoF5, MoF6, NdF3, NpF4, NpF5, NpF6, NiF2, NbF4, NbF5, ONF3, NF3, NO2BF4, NOBF4, NOF, NO2F, OsF4, OsF6, OsF7, OF2, PdF2, PdF4, FSO2OOSO2F, POF3, PF5, PF3, PtF2, PtF4, PtF6, PuF3, PuF4, PuF6, KF, KPF6, KBF4, PrF3, PaF5, RaF2, RnF2, ReF4, ReF6, ReF7, RhF3, RbF, RuF3, RuF4, RuF6, SmF3, ScF3, SeF6, SeF4, SiF4, AgF, AgF2, AgBF4, NaF, FSO3Na, Na3AlF6, NaSbF6, NaPF6, Na2SiF6, Na2TiF6, NaBF4, SrF2, SF2, SF6, SF4, SO2F2, TaF5, TcF6, TeF6, TeF4, TlF, TlF3, SOF2, ThF4, SnF2, SnF4, TiF3, TiF4, HSiF3, WF6, UF4, UF5, UF6, UO2F2, VF3, VF4, VF5, XeF2, XeO2F2, XeF6, XePtF6, XeF4, YbF3, YF3, ZnF2, ZrF4, Fr2O, FrF, FrCl, FrBr, FrI Fr2CO3, FrOH, Fr2SO4, Gd2(CO3)3, GdCl3, GdF3, Gd3Ga5O12, Gd(NO3)3, Gd2O3, GdPO4, Gd2(SO4)3 AlGaAs2, Gd3Ga5O12, GaH3, GaBr3, GaCl3, GaF3, Ga(OH)3, Ga(NO3)3, GaN, Ga2O3, GaP, Ga2(SO4)3, Ga2S3, Me3Ga, GeH4, GeCl2, GeI2, GeO2, GeSe2, GeS4, GeS, GeCl4, GeF4, (CH3)4Ge, (C2H5)3GeCl, HAuCl4, (Ph3P)AuCl, AuBr3, AuCl, AuCl3, Au2O3, Au2S, KAu(CN)2, HfC HfO2, HfCl4, Cp2HfCl2, He, Ho2(CO3)3, HoCl3, HoF3, Ho(NO3)3, Ho2O3, HoPO4, Ho2(SO4)3, Hydrogen (Protium,Deuterium,Tritium), AlH3, NH3, AsH3, BeH2, BiH3, H3BO3, HBrO3, CaH2, H2C2B10H10, HClO3, HClO2, B10H14, N2H2, P2H4, HBF4, GaH3, GeH4, HSbF6, HPF6, H2SiF6, H2TiF6, N2H4, HN3, HBr(aq), HCl(aq), HCN, HF(aq) HBr, HCl, HF, HI, H2O2, H2Se, H2S, H2Te, HI(aq), HOBr(aq), HOCl(aq), H3PO2, LiAlH4, LiBH4, LiH, HNO3, HNO2, B5H9, B5H11, HClO4, H2S2O8, H2SO5, H3PO4, H3PO3, KH, H2P2O7, H2SeO4, SiH4, H2SiO3, NaBH4, NaH, SnH4, SbH3, H2SO4, H2SO3, H2TeO4, B4H10, Zn(BH4)2, In(OAc)3, InSb, InAs InBr3, InCl3, InF3, InI, In(NO3)3, InN, In2O, In2O3, InP, In2Se3, In2(SO4)3, In2S3, In(CH3)3, AlI3, NH4I, BaI2, BI3, CsI, CaI2, CI4, CuI, P2I4, GeI2, HI(aq), HI(g), InI, HIO3, IBr, IC, I2O5, IF7 IF5, IF3, FeI2, PbI2, Li, MgI2, Hg2I2, HgI2, PI3, KI, KIO3, KIO4, SmI4, AgI, NaI, NaIO3, NaIO4, TlI, SnI2, ZnI2, (IrCl(1,5-cod)2)2, Ir4(CO)12, IrCl3, IrCl4, IrO2, Na2IrCl6, AcC5H4FeCp, (NH4)2Fe(SO4)2, dppf, FeCp*2, C6H8Fe(CO)3, (CpFe(CO)2)2, FeCp2, Fe(OAc)2, Fe(OAc)3, Fe(acac)3, FeBr2, FeBr3, FeCl2, FeCl3, FeS2, Fe3(CO)12, FeF3, [[Iron(II) fumarate|C4H2FeO4]], FeI2, Fe(ONap)3, Fe(NO3)3, Fe2(CO)9, FeC2O4, FeO, Fe3O4, Fe2O3, Fe(CO)5, Fe(ClO4)3, FePO4, (NH2SO3)2Fe, FeSO4, Fe2(SO4)3, FeS, Kr, KrF2, La(OAc)3, LaB6, La2(CO3)3, LaCl3, LaF3, La2O3, La(NO3)3, LaPO4, La2(SO4)3, PbCO3, PbCl2, PbCl4, PbO2, PbF2, PbI2, Pb(CH3)4, Pb(C2H5)4, Pb(NO3)2, PbO, PbSO4, PbS, PbSe, PbTe, LiH, Li3N, LiNH2, Li2O, LiOH, LiF, LiCl, LiBr, LiI Li2CO3, Li2SO4, LuBr3, Lu2(CO3)3, LuCl3, LuF3, LuI3, Lu(NO3)3, Lu2O3, LuPO4, Lu2(SO4)3, Lu2S3, Mg(OCOCH3)2, Mg(HCO3)2, MgB6, MgBr2, MgC2, MgCO3, MgCl2, Magnesium citrate, MgNCN, MgF2, MgPO3F, Mg(HOCH2(CHOH)4CO2)2, MgH2, MgHPO4, Mg(OH)2, Mg(OCl)2, MgI2, MgMoO4, Mg(NO3)2, MgC2O4, MgO, MgO2, Mg3(PO4)2, MgSiO3, MgSO4, MgS, MgTiO3, MgWO4, MgZrO3, Mn, MnO2, KMnO4, MnPc, Md, HgO, HgS, HgSe, HgTe, HgCdTe, HgF2, HgCl2, Hg2Cl2, HgBr2, HgI2 Hg(OH)2, Hg2SO4, HgSO4, (CH3)2Hg, MoCl5, Mo(CO)6, MoO3, MoS2, Nd, NdI3, NpCl3, NpCl4, NpCl5, NpCl6, NpF4, NpF5, NpF6, NpO2, Nb, Nb2O5, N, NH3, N2H4, HNO3, HNO2, N2O, Os, OsO4, Al2O3, AmO, AmO2, Sb2O3, Sb2O5, As2O3, As2O5, BaO, BeO, Bi2O3, BiOCl, B2O3, Br2O, CO2, CO, CeO2, ClO2, ClO3, Cl2O7, Cl2O, Cr2O3, CrO2, CrO3, CoO, Cu2O, CuO, Cm2O3, CmO2, Dy2O3, Er2O3, Eu2O3, OF2, O2F2, Fr2O, Gd2O3, Ga2O3, GeO2, Au2O3, HfO2, Ho2O3, In2O, In2O3, I2O5, IrO2, FeO, Fe3O4, Fe2O3, La2O3, PbO, PbO2, Li2O, MgO, K2O, Rb2O, Na2O, SrO, TeO2, UO2, PdCl2, PBr3, PCl3, PF5, P2O3, P2O5, P4S10, P4S3, PH3, H3PO4, H2PO3, KPF6, K3PO4, K2HPO4, KH2PO4, (C6H5)3P (CH3)3P, Pt, Pu, Po, K2O, KF, KCl, KBr, KI K2CO3, KOH, K2SO4, KMnO4, K2FeO4, KAl(SO4)2, Potassium citrate, KHCO3, KNO3, KVO3, K2Cr2O7, K2CrO4, K2S, KSb(OH)6, K3[Fe(CN)6], K4[Fe(CN)6], K2PtCl6, PrBr3, Pr2(CO3)3, PrCl3, PrF3, PrI3, Pr(NO3)3, Pr2O3, PrPO4, Pr2(SO4)3, Pr2S3, PmBr3, Pm2(CO3)3, PmCl3, PmF3, PmI3, Pm(NO3)3, Pm2O3, PmPO4, Pm2(SO4)3, Pm2S3, PaCl3, PaCl4, PaCl5, PaCl6, PaF4, PaF5, PaF6, PaO2, Ra(OCOCH3)2, Ra(HCO3)2, RaB6, RaBr2, RaC2, RaCO3, RaCl2, Radium citrate, RaNCN, RaF2, RaPO3F, Ra(HOCH2(CHOH)4CO2)2, RaH2, RaHPO4, Ra(OH)2, Ra(OCl)2, RaI2, RaMoO4, Ra(NO3)2, RaC2O4, RaO, RaO2, Ra3(PO4)2, RaSiO3, RaSO4, RaS, RaTiO3, RaWO4, RaZrO3, RnF2, RhB2, Rg, RbH, Rb2O, RbF, RbCl, RbBr, RbI, Rb2Te, Rb2CO3, RbOH, Rb2SO4, Rb2ClO4, Ru, Rf, SmBr3, Sm2(CO3)3, SmCl3, SmF3, SmI3, Sm(NO3)3, Sm2O3, SmPO4, Sm2(SO4)3, Sm2S3, ScBr3, Sc2(CO3)3, ScCl3, ScF3, ScI3, Sc(NO3)3, Sc2O3, ScPO4, Sc2(SO4)3, Sc2S3, CdSe, H2Se, HgSe, SeO2, H2SeO4, H2SeO3, Na2SeO3, ZnSe, Si, SiC, Silicon carbide SiN, Silicon nitride SiO2, Silicon dioxide, AgF, AgF2, AgBr, AgCl, AgI, AgCN, AgONC, AgNO3, AgClO4, Ag2S, Ag2Te, Ag2O, AgO, AgBF4, NaHCO3, NaBr, Na2CO3, NaCl, Na4Fe(CN)6, NaF, NaOH, NaI, Na2O, Na2SO4, Sr(OCOCH3)2, Sr(HCO3)2, SrB6, SrBr2, SrC2, SrCO3, SrCl2, Strontium citrate, SrNCN, SrF2, SrPO3F, Sr(HOCH2(CHOH)4CO2)2, SrH2, SrHPO4, Sr(OH)2, Sr(OCl)2, SrI2, SrMoO4, Sr(NO3)2, SrC2O4, SrO, SrO2, Sr3(PO4)2, SrSiO3, SrSO4, SrS, SrTiO3, SrWO4, SrZrO3, NaSH, H3NSO3, H2S, SO2, H2SO4, H2SO3, SO2Cl2, CdS, HgS, ZnS, S2Cl2, SCl2, SF4, SOCl2, SOBr2, CS2, CSCl2, MoS2, NH4)2S HS(Hydrogen sulphide), Ta, BeTe, Bi2Te3, CdTe, (Cd,Zn)Te, (CH3)2Te, (Hg,Cd)Te, PbTe, HgTe, (Hg,Zn)Te, Ag2Te, SnTe, ZnTe, HOTeF5, H6TeO6, Na2TeO3, TeO2, TeF6, TeF4.TeCl4, TbBr3, Tb2(CO3)3, TbCl3, TbF3, TbI3, Tb(NO3)3, Tb2O3, TbPO4, Tb2(SO4)3, Tb2S3, Tl(OAc)3, TlSb, TlAs TlBr3, TlCl3, TlF3, TlI, Tl(NO3)3, Tl2O, Tl2O3, TIP, Tl2Se3, Tl2(SO4)3, Tl2S3, Tl(CH3)3, TlOH, ThCl3, ThCl4, ThCl5, ThCl6, ThF4, ThF5, ThF6, ThO2, TmBr3, Tm2(CO3)3, TmCl3, TmF3, TmI3, Tm(NO3)3, Tm2O3, TmPO4, Tm2(SO4)3, Tm2S3, SnF2, SnCl2, SnCl4, SnO, SnO2, Sn(CH3)4, Sn(C2H5)4, SnS, SnSe, SnTe, TiC, TiN, Titanium nitride TiCl2, TiCl3, TiO2, TiBr4, TiCl4, TiI4, Cp2TiCl2, UCl3, UCl4, UCl5, UCl6, UF4, UF5, UF6, UO2F2, UO4, UO2, VC, VBr3, VCl2, VCl3 VCl4, VOCl3, V2O5 Cp2VCl2, XeF2, XePtF6, XeF4, XeF6, XeO4, YbBr3, Yb2(CO3)3, YbCl3, YbF3, YbI3, Yb(NO3)3, Yb2O3, YbPO4, Yb2(SO4)3, Yb2S3, YBr3, Y2(CO3)3, YCl3, YF3, YI3, Y(NO3)3, Y2O3, YPO4, Y2(SO4)3, Y2S3, ZnO, ZnS, ZnSe, ZnTe, ZnF2, ZnCl2, ZnBr2, ZnI2 Zn(OH)2, ZnSO4, ZrC, ZrO2, ZrN, ZrCl4, ZrS2, ZrSi2, ZrSiO4, ZrF4, ZrBr4, ZrI4, Zr(OH)4, C10H11ClZr, Zr(CH3CH2COO)4, Zr(WO4)2, ZrH2, Pb(ZrxTi1-x)O3.

Preferred characteristics of library molecules are described in the following:

### Molecular Weight

Large molecules can provide many potential contact points to a surface or material and thus the highest affinity ligands may be found among large molecules. In cases where a high affinity is the most important characteristic, a high MW of a library molecule is preferred. Thus, depending on the context, the MW as measured in Dalton is preferably greater than 10, such as greater than 100, such as greater than 10², such as greater than 10³, such as greater than 10⁴, such as greater than 10⁵, such as greater than 10⁶, such as greater than 10⁸, such as greater than 10⁹. In other cases where cost of molecule synthesis is important, a low MW is the most important characteristic of a library molecule. Thus, depending on context, the MW of library molecules measured in Dalton is preferably lower than 10⁹, such as lower than 10⁸, such as lower than 10⁷, such as lower than 10⁶, such as lower than 10⁵, such as lower than 10⁴, such as lower than 10³, such as lower than 10², such as lower than 10. In cases where both affinity and cost are important characteristics, the MW measured in Dalton is preferably; 10¹ to 10², or 10² to 10³, or 10³ to 10⁴, or 10⁴ to 10⁵, or 10⁵ to 10⁶, or 10⁶ to 10⁷, or 10⁷ to 10⁸, or 10⁸ to 10⁹.

### Atomic Composition

For any characteristic of a library molecule mentioned above, and in each characteristic's entire range, a further characteristic of importance is the atomic composition of the library molecule. In some cases, molecules comprising a single type of atom are preferred. For example, when fullerene-binding ligands are sought, molecules are preferably composed of carbon; e.g. CNT fragments, and graphene fragments. As another example, when hydrophobic ligands are sought, molecules preferably are composed of C and H, e.g. such as polyethylene or polypropylene. When hydrophilic ligands are sought, molecules preferably are composed of C, O and H, e.g. such as polyethyleneglycol. As another example, when ligands comprising both hydrogen bond donors and acceptors are sought, molecules preferably comprise C, N, H, and O, e.g. such as peptides or small molecules.

### Hydrogen Bond Donors (HBD)

For any characteristic of a library molecule mentioned above, and in each characteristic's entire range, a further characteristic of importance is the number of hydrogen bond donors of the library molecule. In cases where ligands to materials with a high number of hydrogen bond acceptors are sought, a high number of HBD is the most important characteristic of the molecule library. Thus, depending on context, the number of HBD of library molecules is preferably more than 0, such as more than 1, such as more than 2, such as more than 3, such as more than 4, such as more than 5, such as more than 6, such as more than 7, such as more than 8, such as more than 9, such as more than 12, such as more than 15, such as more than 18, such as more than 21, such as more than 24, such as more than 27, such as more than 30, such as more than 33, such as more than 36, such as more than 39, such as more than 42, such as more than 45, such as more than 48, such as more than 51, such as more than 54, such as more than 57, such as more than 60, such as more than 63, such as more than 66, such as more than 69, such as more than 72, such as more than 75, such as more than 78, such as more than 81, such as more than 84, such as more than 87, such as more than 90, such as more than 93, such as more than 96, such as more than 99. In cases where ligands to materials with a low number of hydrogen bond acceptors are sought, a low number of HBD is the most important characteristic of the molecule library. Thus, depending on context, the number of HBD of library molecules is preferably more than 99, such as less than 96, such as less than 93, such as less than 90, such as less than 87, such as less than 84, such as less than 81, such as less than 78, such as less than 75, such as less than 72, such as less than 69, such as less than 66, such as less than 63, such as less than 60, such as less than 57, such as less than 54, such as less than 51, such as less than 48, such as less than 45, such as less than 42, such as less than 39, such as less than 36, such as less than 33, such as less than 30, such as less than 27, such as less than 24, such as less than 21, such as less than 18, such as less than 15, such as less than 12, such as less than 9, such as less than 8, such as less than 7, such as less than 6, such as less than 5, such as less than 4, such as less than 3, such as less than 2, such as less than 1.

### Hydrogen Bond Acceptors (HBA)

For any characteristic of a library molecule mentioned above, and in each characteristic's entire range, a further characteristic of importance is the number of hydrogen bond acceptors of the library molecule. In cases where ligands to materials with a high number of hydrogen bond donors are sought, a high number of HBA is the most important characteristic of the molecule library. Thus, depending on context, the number of HBA of library molecules is preferably more than 1, such as more than 2, such as more than 3, such as more than 4, such as more than 5, such as more than 6, such as more than 7, such as more than 8, such as more than 9, such as more than 11, such as more than 13, such as more than 15, such as more than 17, such as more than 19, such as more than 21, such as more than 23, such as more than 25, such as more than 27, such as more than 29, such as more than 34, such as more than 39, such as more than 44, such as more than 49, such as more than 54, such as more than 59, such as more than 64, such as more than 69, such as more than 74, such as more than 79. In cases where ligands to materials with a low number of hydrogen bond donors are sought, a low number of HBAs is the most important characteristic of the molecule library. Thus, depending on context, the number of HBAs of library molecules is preferably less than 94, such as less than 89, such as less than 84, such as less than 79, such as less than 74, such as less than 69, such as less than 64, such as less than 59, such as less than 54, such as less than 49, such as less than 44, such as less than 39, such as less than 34, such as less than 29, such as less than 27, such as less than 25, such as less than 23, such as less than 21, such as less than 19, such as less than 17, such as less than 15, such as less than 13, such as less than 11, such as less than 9, such as less than 8, such as less than 7, such as less than 6, such as less than 5, such as less than 4

### Rotatable Bonds

For any characteristic of a library molecule mentioned above, and in each characteristic's entire range, a further characteristic of importance is the number of rotatable bonds of the library molecule. Molecules with fewer rotatable bonds typically are more rigid. Two molecules, carrying similar chemical functionalities (like amino groups, thiols, etc) but of different rigidity will display different likelihood of binding to surfaces. Thus, the flexible molecule can adapt many alternative overall structures, and can therefore bind to many different kinds of surfaces. However, because of its high flexibility, there is a large entropy loss upon binding to most surfaces, and the affinity achieved for any given surface is rarely very high. The rigid molecule, on the other hand, has little entropy loss upon binding, but does not have the adaptability displayed by the flexible molecule. As a result, the rigid molecule will typically have very low affinity to most surfaces, but high affinity for those few surfaces where the rigid structure has a nice fit. Thus, in cases where a high affinity is sought, it will generally be advantageous to use libraries comprising many rigid molecules, i.e. molecules with few rotatable bonds. In cases where many different molecules of mediochre affinity is sought, it may be advantageous to use libraries comprising many flexible molecules (i.e. molecules with many rotatable bonds). For the same reasons, in cases where molecules are sought that can bind to different (but similar) surfaces, it may also be advantageous to use libraries comprising many molecules with many rotatable bonds. Of course, when molecules are sought that can both bind with relatively high affinity to the individual surface, but also bind to different (but similar) surfaces, the appropriate compromise will be to use libraries comprising many molecules that are neither very flexible nor very rigid. In cases where flexible ligands are sought, a high number of rotatable bonds is the most important characteristic of the molecule library. Thus, depending on context, the number of rotatable bonds of library molecules is preferably more than 1, such as more than 2, such as more than 3, such as more than 4, such as more than 5, such as more than 6, such as more than 7, such as more than 8, such as more than 9, such as more than 10, such as more than 11, such as more than 12, such as more than 13, such as more than 14, such as more than 15, such as more than 16, such as more than 17, such as more than 18, such as more than 19, such as more than 20, such as more than 21, such as more than 22, such as more than 23, such as more than 24, such as more than 25, such as more than 26, such as more than 27, such as more than 28, such as more than 29. In cases where rigid ligands are sought, e.g. to ensure a high affinity to a material, a low number of rotatable bonds is the most important characteristic of the molecule library. Thus, depending on context, the number of rotatable bonds of library molecules is preferably less than 30, such as less than 29, such as less than 28, such as less than 27, such as less than 26, such as less than 25, such as less than 24, such as less than 23, such as less than 22, such as less than 21, such as less than 20, such as less than 19, such as less than 18, such as less than 17, such as less than 16, such as less than 15, such as less than 14, such as less than 13, such as less than 12, such as less than 11, such as less than 10, such as less than 9, such as less than 8, such as less than 7, such as less than 6, such as less than 5, such as less than 4, such as less than 3, such as less than 2.

### Number of Atoms

For any characteristic of a library molecule mentioned above, and in each characteristic's entire range, a further characteristic of importance is the number of atoms of the library molecule. In cases where large molecules are preferred a high number of atoms is the most important characteristic of the molecule library. Thus, depending on context, the number of atoms of library molecules is preferably more than 2, such as more than 4, such as more than 6, such as more than 10, such as more than 16, such as more than 26, such as more than 42, such as more than 67, such as more than 107, such as more than 172, such as more than 275, such as more than 440, such as more than 704, such as more than 1126, such as more than 1801, such as more than 2882. In other cases where small molecules are preferred, e.g. to reduce cost of synthesis, a low number of atoms is the most important characteristic of the molecule library. Thus, depending on context, the number of library molecules is preferably less than 2882, such as less than 1801, such as less than 1126, such as less than 704, such as less than 440, such as less than 275, such as less than 172, such as less than 107, such as less than 67, such as less than 42, such as less than 26, such as less than 16, such as less than 10, such as less than 6, such as less than 4, such as less than 2.

### Charge

For any characteristic of a library molecule mentioned above, and in each characteristic's entire range, a further characteristic of importance is the charge of the library molecule. In cases where ligands with high affinity to negatively charged materials are sought, a high positive charge is the most important characteristic of the molecule library. Thus, depending on context, the charge is preferably higher than −50, such as higher than −45, such as higher than −40, such as higher than −35, such as higher than −30, such as higher than −25, such as higher than −20, such as higher than −15, such as higher than −10, such as higher than −5, such as higher than 0, such as higher than 5, such as higher than 10, such as higher than 15, such as higher than 20, such as higher than 25, such as higher than 30, such as higher than 35, such as higher than 40, such as higher than 45, such as higher than 50. In cases where ligands with high affinity to positively charged materials are sought, a high negative charge is the most important characteristic of the molecule library. Thus, depending on context, the charge is preferably lower than 50, such as lower than 45, such as lower than 40, such as lower than 35, such as lower than 30, such as lower than 25, such as lower than 20, such as lower than 15, such as lower than 10, such as lower than 5, such as lower than 0, such as lower than −5, such as lower than −10, such as lower than −15, such as lower than −20, such as lower than −25, such as lower than −30, such as lower than −35, such as lower than −40, such as lower than −45, such as lower than −50.

cLogP, Calculated Logarithm of the Octanol-Water Partition Coefficient

For any characteristic of a library molecule mentioned above, and in each characteristic's entire range, a further characteristic of importance is the cLogP of the library molecule. In cases where hydrophopic ligands are sought, e.g. for binding to a hydrophobic surface, a high cLogP is the most important characteristic of the molecule library. Thus, depending on context, the cLogP of library molecules is preferably higher than −100, such as higher than −90, such as higher than −80, such as higher than −70, such as higher than −60, such as higher than −50, such as higher than −40, such as higher than −30, such as higher than −20, such as higher than −10, such as higher than 0, such as higher than 10, such as higher than 20, such as higher than 30, such as higher than 40, such as higher than 50, such as higher than 60, such as higher than 70, such as higher than 80, such as higher than 90. In cases where hydrophilic ligands are sought, e.g. for binding to a hydrophilic surface, a low cLogP is the most important characteristic of the molecule library. Thus, depending on context, the cLogP of library molecules is preferably lower than 90, such as lower than 80, such as lower than 70, such as lower than 60, such as lower than 50, such as lower than 40, such as lower than 30, such as lower than 20, such as lower than 10, such as lower than 0, such as lower than −10, such as lower than −20, such as lower than −30, such as lower than −40, such as lower than −50, such as lower than −60, such as lower than −70, such as lower than −80, such as lower than −90, such as lower than −100.

### Polar Surface Area (PSA)

For any characteristic of a library molecule mentioned above, and in each characteristic's entire range, a further characteristic of importance is the polar surface area of the library molecule. In cases where large polar ligands are sought, e.g. to achieve efficient binding to a polar surface, a high PSA is the most important characteristic of the molecule library. Thus, depending on context, the PSA of library molecules measure in square Angstrom is preferably more than 10, such as more than 100, such as more than 160, such as more than 256, such as more than 410, such as more than 655, such as more than 1049, such as more than 1678, such as more than 2684, such as more than 4295, such as more than 6872, such as more than 10995, such as more than 17592, such as more than 28147, such as more than 45036, such as more than 72058, such as more than 115292, such as more than 184467, such as more than 295148, such as more than 472237, such as more than 755579. In cases where small polar ligands are sought, e.g. to achieve a low cost of production of a ligand, a low PSA is the most important characteristic of the molecule library. Thus, depending on context, the PSA of library molecules measured in square Angstrom is preferably less than 755579, such as less than 472237, such as less than 295148, such as less than 184467, such as less than 115292, such as less than 72058, such as less than 45036, such as less than 28147, such as less than 17592, such as less than 10995, such as less than 6872, such as less than 4295, such as less than 2684, such as less than 1678, such as less than 1049, such as less than 655, such as less than 410, such as less than 256, such as less than 160, such as less than 100, such as less than 10.

### Molecular Weight

For any characteristic of a library molecule mentioned above, and in each characteristic's entire range, a further characteristic of importance is molecular weight of the library molecule. Large molecules can provide many potential contact points to a surface or material and thus the highest affinity ligands may be found among large molecules. In cases where a high affinity is the most important characteristic, a high MW is preferred. Thus, depending on the context, the MW as measured in Dalton is preferably greater than 10, such as greater than 100, such as greater than 10², such as greater than 10³, such as greater than 10⁴, such as greater than 10⁵, such as greater than 10⁶, such as greater than 10⁸, such as greater than 10⁹. In other cases where cost of library synthesis is important, a low MW is the most important characteristic of the molecule library. Thus, depending on context, the MW of library molecules measured in Dalton is preferably lower than 10⁹, such as lower than 10⁸, such as lower than 10⁷, such as lower than 10⁶, such as lower than 10⁵, such as lower than 10⁴, such as lower than 10³, such as lower than 10², such as lower than 10. In cases where both affinity and cost are important characteristics, the MW measured in Dalton is preferably; 10¹ to 10², or 10² to 10³, or 10³ to 10⁴, or 10⁴ to 10⁵, or 10⁵ to 10⁶, or 10⁶ to 10⁷, or 10⁷ to 10⁸, or 10⁸ to 10⁹.

### Atomic Elements

For any characteristic of a molecule mentioned above or below, and in each characteristic's entire range, a further important characteristic of a molecule is the number of different atomic elements. Molecules of the present invention may be characterized by their number of different elements.

In preferred embodiments, said molecules are composed of a single element or two elements or three elements or four elements or five elements or six elements or seven elements or eight elements or nine elements or ten elements chosen from: Hydrogen (H), Helium (He), Lithium (Li), Beryllium (Be), Boron (B), Carbon (C), Nitrogen (N), Oxygen (O), Fluorine (F), Neon (Ne), Sodium (Na), Magnesium (Mg), Aluminium (Al), Silicon (Si), Phosphorus (P), Sulfur (S), Chlorine (Cl), Argon (Ar), Potassium (K), Calcium (Ca), Scandium (Sc), Titanium (Ti), Vanadium (V), Chromium (Cr), Manganese (Mn), Iron (Fe), Cobalt (Co), Nickel (Ni), Copper (Cu), Zinc (Zn), Gallium (Ga), Germanium (Ge), Arsenic (As), Selenium (Se), Bromine (Br), Krypton (Kr), Rubidium (Rb), Strontium (Sr), Yttrium (Y), Zirconium (Zr), Niobium (Nb), Molybdenum (Mo), Technetium (Tc), Ruthenium (Ru), Rhodium (Rh), Palladium (Pd), Silver (Ag), Cadmium (Cd), Indium (In), Tin (Sn), Antimony (Sb), Tellurium (Te), Iodine (I), Xenon (Xe), Caesium (Cs), Barium (Ba), Lanthanum (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Hafnium (Hf), Tantalum (Ta), Tungsten (W), Rhenium (Re), Osmium (Os), Iridium (Ir), Platinum (Pt), Gold (Au), Mercury (Hg), Thallium (Tl), Lead (Pb), Bismuth (Bi), Polonium (Po), Astatine (At), Radon (Rn), Francium (Fr), Radium (Ra), Actinium (Ac), Thorium (Th), Protactinium (Pa), Uranium (U), Neptunium (Np), Plutonium (Pu)

### Molecule State

In preferred embodiments, efficient interaction between molecules and surface requires a liquid medium and molecules are in a liquid state. In other preferred embodiments, molecules are in the gaseous state, e.g. during mass spectrometry. A molecule of the present invention may at any point in time or period of time, be in a liquid state, a gaseous state, a solid state, a crystalline state, or a plasma state.

Important library characteristics are discussed in the following:

### Total Number of Molecules

To reduce the risk that molecules are stochastically lost during library handling, including partitioning steps, it is important to have a sufficiently high number of each molecule present in the library. Furthermore, to improve the chance of finding molecules with desired characteristics when screening a library it is important to have many different molecules in a library, i.e to have a high diversity. When both the number of each molecule and the number of different molecules is high, the total number of molecules in the library will be high. Thus, depending on context, the total number of molecules is preferably more than 10, such as more than 100, such as more than 10², such as more than 10³, such as more than 10⁴, such as more than 10⁵, such as more than 10⁶, such as more than 10⁷, such as more than 10⁸, such as more than 10⁹, such as more than 10¹⁰, such as more than 10¹¹, such as more than 10¹², such as more than 10¹³, such as more than 10¹⁴, such as more than 10¹⁵, such as more than 10¹⁶, such as more than 10¹⁷, such as more than 10¹⁸, such as more than 10¹⁹, such as more than 10²⁰, such as more than 10²¹, such as more than 10²², such as more than 10²³, such as more than 10²⁴, such as more than 10²⁵, such as more than 10²⁶, such as more than 10²⁷, such as more than 10²⁸, such as more than 10²⁹, such as more than 10³⁰, such as more than 10³¹, such as more than 10³², such as more than 10³³, such as more than 10³⁴, such as more than 10³⁵, such as more than 10³⁶, such as more than 10³⁷, such as more than 10³⁸, such as more than 10³⁹. On the other hand, to reduce cost of producing a library, it is important to limit the number of each molecule and the number of different molecules in the library. If both the numbers are low, the total number of library molecules will be low. Thus, depending on context, the total number of molecules in a library is preferably less than 10³⁹, such as less than 10³⁸, such as less than 10³⁷, such as less than 10³⁶, such as less than 10³⁵, such as less than 10³⁴, such as less than 10³³, such as less than 10³², such as less than 10³¹, such as less than 10³⁰, such as less than 10²⁹, such as less than 10²⁸, such as less than 10²⁷, such as less than 10²⁶, such as less than 10²⁵, such as less than 10²⁴, such as less than 10²³, such as less than 10²², such as less than 10²¹, such as less than 10²⁰, such as less than 10¹⁹, such as less than 10¹⁸, such as less than 10¹⁷, such as less than 10¹⁶, such as less than 10¹⁵, such as less than 10¹⁴, such as less than 10¹³, such as less than 10¹², such as less than 10¹¹, such as less than 10¹⁰, such as less than 10⁹, such as less than 10⁸, such as less than 10⁷, such as less than 10⁶, such as less than 10⁵, such as less than 10⁴, such as less than 10³, such as less than 10², such as less than 10.

Similarly, the number of linkers and tags used in a screening is important. In cases where a high number of bacteriophages (linkers) or DNA tags in the selection output is the most important characteristic or where the conditions during the selection results in low recovery (of DNA tags) or survival (of bacteriophage), a high number of linkers, e.g. bacteriophages, or tags, e.g. DNA tags, will be required. Thus, depending on the context, the number of bacteriophages or DNA tags is preferably greater than 1E+1, such as greater than 1E+2, such as greater than 1E+3, such as greater than 1E+4, such as greater than 1E+5, such as greater than 1E+6, such as greater than 1E+7, such as greater than 1E+8, such as greater than 1E+9, such as greater than 1E+10, such as greater than 1E+11, such as greater than 1E+12, such as greater than 1E+13, such as greater than 1E+14, such as greater than 1E+15, such as greater than 1E+16. In cases where a low number of bacteriophages (linkers) or DNA tags in the selection output is the most important characteristic or where the conditions during the selection results in high recovery (of DNA tags) or high level of survival (of bacteriophage), a low number of linkers, e.g. bacteriophages, or tags, e.g. DNA tags, will be required. Thus, depending on the context, the number of bacteriophages or DNA tags is preferably less than 1E+16, such as less than 1E+15, such as less than 1E+14, such as less than 1E+13, such as less than 1E+12, such as less than 1E+11, such as less than 1E+10, such as less than 1E+9, such as less than 1E+8, such as less than 1E+7, such as less than 1E+6, such as less than 1E+5, such as less than 1E+4, such as less than 1E+3, such as less than 1E+2, such as less than 1E+1.

### Affinity

In some cases it is an advantage to include in the library one or more library molecule(s) with known affinity(ies) towards a material/surface. For example, when optimizing screening conditions a library molecule with a known affinity can be analyzed and the data can be used to change screening conditions in order to optimize them. This principle can be expanded, such that the library can include a range of molecules with known affinities that span a range of affinities. In this way, when performing library screening under different conditions in parallel, analysis of screening outputs, e.g. the fraction of a given molecule before and after screening, can be used to guide optimization of library screening conditions. Thus in cases where a library is to be used for optimizing enrichment of high affinity binders towards a material, the presence—in the library—of a molecule with high affinity to a material such as a fullerene such as a CNT or a GS, or a nanotube such as a SWNT or a MWNT or a BNNT will be required. Thus, depending on the context, the affinity of said high affinity binder measured in moles per liter is preferably higher than 10, such as higher than 100, such as higher than 10⁻², such as higher than 10⁻³, such as higher than 10⁻⁴, such as higher than 10⁻⁵, such as higher than 10⁻⁶, such as higher than 10⁻⁷′ such as higher than 10⁻⁸, such as higher than 10⁻⁹, such as higher than 10⁻¹⁰, such as higher than 10⁻¹¹, such as higher than 10⁻¹², such as higher than 10⁻¹³, such as higher than 10⁻¹⁴, such as higher than 10⁻¹⁵, such as higher than 10⁻¹⁶, such as higher than 10⁻¹⁷, such as higher than 10⁻¹⁸, such as higher than 10⁻¹⁹. Thus in cases where a library is to be used for optimizing enrichment of low affinity binders towards a material, the presence—in the library—of a molecule with low affinity to a material will be required.

Thus, depending on the context, the affinity of said high affinity binder measured in moles per liter is preferably lower than 10⁻¹⁸, such as lower than 10⁻¹⁷, such as lower than 10⁻¹⁶, such as lower than 10⁻¹⁵, such as lower than 10⁻¹⁴, such as lower than 10⁻¹³, such as lower than 10⁻¹², such as lower than 10⁻¹¹ such as lower than 10⁻¹⁰, such as lower than 10⁻⁹, such as lower than 10⁻⁸, such as lower than 10⁻⁷ such as lower than 10⁻⁶, such as lower than 10⁻⁵, such as lower than 10⁻⁴, such as lower than 10⁻³, such as lower than 10⁻², such as lower than 10.

In cases where a library is screened to identify molecules which bind a carbon material with a high affinity, the most important characteristic of the library is the presence of one or more molecules with a high affinity for a material chosen from; fullerenes; Buckyball; Buckypaper; Buckytube; Dodecahedrane; Endohedral fullerenes; Gedodesic carbon domes; Prismane; Carbon nanotube; Single-wall carbon nanotubes; Double-wall carbon nanotubes; Triple-wall carbon nanotube; Multi-wall carbon nanotubes; Pristine carbon nanotubes; Coated carbon nanotubes; Defect-free carbon nanotubes; Defect carbon nanotubes; Functionalized carbon nanotubes comprising a Thiol, a Hydroxyl, Carboxylic acid, or an Amine; (Fullerite; Ultrahard fullerite; Buckminsterfullerene; Graphene; Single-layer graphene; Double-layer graphene; Triple-layer graphene; Multi-layer graphene; Pristine graphene; Coated graphene; Defect-free graphene; Defect graphene; Functionalized graphene; Graphane; Flourinated graphene; Graphene oxide; Reduced graphene oxide; Buckyball clusters; Carbon megatubes; Carbon polymers; Carbon nano-onions; Carbon nanobuds, Fullerene rings, carbon fiber, carbon nanofiber. Defects in carbon nanotubes or graphene include; adducts, vacancies (missing carbon atoms), a 5-7 defect, a 5-7-7-5 Stone-Wales defect.

The above mentioned preferred characteristics of individual library molecules also apply to the library as a whole. For example, the library will have an average MW, average number of HBD, HPA, and average PSA. Thus, for any characteristic of a library molecule mentioned above, and in each characteristic's entire range, the same characteristic is important as a characteristic of the library. For example, in cases where a high average affinity is the most important characteristic of a library, a high average MW of the library is preferred. Thus, depending on the context, the MW as measured in Dalton is preferably greater than 10, such as greater than 100, such as greater than 10², such as greater than 10³, such as greater than 10⁴, such as greater than 10⁵, such as greater than 10⁶, such as greater than 10⁸, such as greater than 10⁹. In other cases where cost of library synthesis is important, a low average MW is the most important characteristic of the molecule library. Thus, depending on context, the average MW of library molecules measured in Dalton is preferably lower than 10⁹, such as lower than 10⁸, such as lower than 10⁷, such as lower than 10⁶, such as lower than 10⁵, such as lower than 10⁴, such as lower than 10³, such as lower than 10², such as lower than 10. In cases where both affinity and cost are important characteristics, the MW measured in Dalton is preferably; 10¹ to 10², or 10² to 10³, or 10³ to 10⁴, or 10⁴ to 10⁵, or 10⁵ to 10⁶, or 10⁶ to 10⁷, or 10⁷ to 10⁸, or 10⁸ to 10⁹.

T, the Tag Linked to the Library Molecule M Via the Linker L

A tag can serve several functions. It may serve as a simple means of identifying the library molecule to which it is attached, see e.g. Brenner S, Lerner R A (June 1992), Proc. Natl. Acad. Sci. U.S.A. 89 (12): 5381-3; Nielsen J, Brenner S, Janda K D (1993), Journal of the American Chemical Society 115 (21): 9812-9813; and Needels et al. (November 1993), Proc. Natl. Acad. Sci. U.S.A. 90 (22): 10700-4. A tag can also serve more sophisticated functions. In the phage display the DNA tag serves as a means of identification, but it also serves as a means to produce more of the isolated peptides, without knowing the identity of the individual peptide molecules. A tag may allow the efficient isolation or identification of molecules with desired characteristics from huge libraries, larger than 10¹² members, see e.g. Roberts R W, Szostak J W. Proc Natl Acad Sci USA. 1997; 94: 12297-12302. In many cases it is important that the chemistry of the tag is orthogonal, or at least does not interfere with, the library molecule. Therefore, in some cases it is advantageous to exclude a tag, as this can allow a more diverse chemistry to be applied to the synthesis of the library molecules.

L, the Linker Connecting the Library Molecule M and the Tag T

In preferred embodiments, molecules are attached to a linker optionally attached to another entity, e.g. a tag. In preferred embodiments, fragments are attached to a linker optionally attached to another entity such as another fragment and/or a tag. In preferred embodiments, a linker suitable for the present invention is linear. In other preferred embodiments, a linker suitable for the present invention is branched. In preferred embodiments, branching linkers have more than two ends or nodes. Said nodes may individually be linked to a molecule or a tag. In preferred embodiments, a linker suitable for the present invention is chosen from: Carbohydrates and substituted carbohydrates, polyvinyl, acetylene or polyacetylene, aryl/hetaryl and substituted aryl/hetaryl, ethers and polyethers such as e.g. polyethylenglycol and substituted polyethers, amines, polyamines and substituted polyamines, single- or double-stranded oligonucleotides, and polyamides and natural and unnatural polypeptides. Said linker is preferably flexible, enabling it to expose the encoded molecule in an optimal way. Preferably the length of the flexible linker is in the range of 1-50 angstroms, more preferably 5-30 angstroms, most preferably 10-25 angstroms. Preferably the linker is both flexible and inert; polyethylene glycol (PEG) is an appropriate linker.

Under certain conditions it may desirable to be able to cleave the linker after the screening of the library of bi-functional molecules has been done, for example in order to perform a mass spectrometric analysis of the encoded molecule without the identifier attached, or to perform other assays on the free encoded molecule.

In an alternative embodiment, the linker contains an oligonucleotide moiety which may serve as an annealing site for an oligonucleotide that carries a reagent, catalyst or molecule fragment. The annealing of the reagent-, catalyst- or molecule fragment-oligonucleotide will serve to provide the reagent, catalyst or molecule fragment in a high local concentration, thereby improving the efficiency of a desired reaction.

In preferred embodiments, the linker is a bead. For example, suitable libraries may be chosen from M-L-F, where M is a peptide or small organic molecule or macrocycle, L is a bead such as a polystyrene bead, and T is a tag such as DNA.

In some embodiments it is preferable that the linker ensures that a reactive group or a building block (reactant) or an encoded molecule is spaced away from the tag. In some embodiments it is also preferable that the linker ensures that a reactive group, a building block (reactant) or an encoded molecule can efficiently interact with another object such as a target used for screening/affinity selection.

The linker may be composed of one or more atoms. The linker may include monomer units such as a peptide, protein, carbohydrates and substituted carbohydrates, a nucleotide, or any unit synthesized using organic and/or inorganic chemistry such as ethylenglycol; 1,3-propylenglycol; 1,4-propylenglycol; 1,5-pentylenglycol. Any unit may be in substituted form, e.g., 1,3.propylenglycol hydroxyl-substituted at the 2 position (Propane-1,2,3-triol). The linker may also include a polymer such as an organic polymer, e.g. a polyethylenglycol, a polypeptide, or an oligonucleotide, polyvinyl, acetylene or polyacetylene, aryl/hetaryl and substituted aryl/hetaryl, ethers and polyethers such as e.g. polyethylenglycol and substituted polyethers, amines, polyamines and substituted polyamines, single- or double-stranded oligonucleotides, and polyamides and natural and unnatural polypeptides.

The linker may contain any combination of monomeric and polymeric units. The linker may also contain branching units. The linker may be flexible or rigid and contain flexible and/or rigid parts. The linker may be attached to one or more reactive groups by one or more atoms. Moreover, the linker may contain one or more reactive groups. The linker may be attached to the tag via one or more atoms, e.g. via a phosphate group.

The attachment point may be anywhere on the tags such as a 5′ or 3′ phosphate, a 5′ or 3′ OH, carbon, oxygen or nitrogen on one or more nucleotides. The linker may be attached one or more tags such as both strands of a double stranded tag. The linker may be attached to the tag by one or more covalent bonds and/or one or more noncovalent bonds, e.g. the linker may include a biotin moiety which can bind noncovalently to a streptavidin molecule attached to the tag. Preferably the length of the linker is in the range of 1-50 angstrom, more preferably 5-30 angstrom, most preferably 10-25 angstrom. Preferably, the linker separates the linker-tag attachment point from a reactive group by 5-50 atomic bonds, more preferably, by 10-30 atomic bonds, most preferably by 15-25 atomic bonds. Preferably, the linker is prepared from Diisopropyl-phosphoramidous acid 2-cyano-ethyl ester 2-[2-(2-{2-[2-(2-{[(4-methoxy-phenyl)-diphenyl-methyl]-amino}-ethoxy)-ethoxy]-ethoxy}-ethoxy)-ethoxy]-ethyl ester or similar compound. Preferably, the linker contains the structure 2-[2-(2-{2-[2-(2-Amino-ethoxy)-ethoxy]-ethoxy}-ethoxy)-ethoxy]-ethanol.

Cleavable linkers can be cleaved in any number of ways, e.g., by photolysis or increased temperature, or by the addition of acid, base, enzymes, ribozymes, other catalysts, or any other agents.

To maintain a physical link between the identifier and the encoded molecule (in the case of stage 2 synthesis, the template and the encoded molecule), at least one non-cleavable linker is needed. The non-cleavable linker may of course be cleavable under certain conditions, but is non-cleavable under the conditions that lead to the bi-functional molecule employed in the screening. This non-cleavable linker is preferably flexible, enabling it to expose the encoded molecule in an optimal way. Under certain conditions it may desirable to be able to cleave the linker before, during or after the screening of the library has been done, for example in order to perform a mass spectrometric analysis of the encoded molecule without the identifier attached, or to perform other types of assays on the free encoded molecule.

The linking moiety in one embodiment separates the priming site from the chemical reaction site so as to allow an enzyme to perform the tag addition and provide for a hybridisation region. The linking moiety can be a nucleic acid sequence, such as an oligonucleotide. The length of the oligonucleotide is preferably suitable for hybridisation with a complementing oligonucleotide, i.e. the number of nucleotides in the linking moiety is suitably 2 or more, such as 3 or more, for example 4 or above, such as 5 or more, for example 6 or more, such as 7 or more, for example 8 or more nucleotides. In a certain embodiment, the linking moiety is attached to the chemical reaction site via a spacer comprising a selectively cleavable linker to enable release of the molecule from the identifier oligonucleotide in a step subsequent to the formation of the final bifunctional complex. The cleavable linker can be selectively cleavable, i.e. conditions can be selected that only cleave that particular linker.

The cleavable linkers can be selected from a variety chemical structures. Examples of linkers includes, but are not limited to, linkers having an enzymatic cleavage site, linkers comprising a chemical degradable component, linkers cleavable by electromagnetic radiation.

Examples of linkers cleavable by electromagnetic radiation (light) o-nitrobenzyl

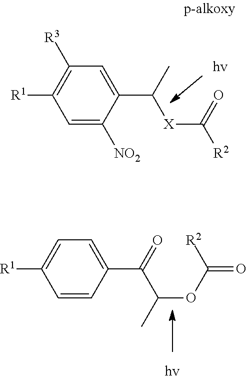

o-nitrobenzyl in exo position

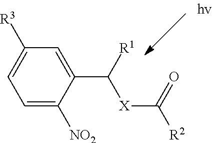

For more details see Holmes C P. J. Org. Chem. 1997, 62, 2370-2380

3-nitrophenyloxy

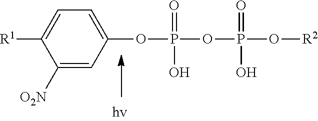

For more details see Rajasekharan Pillai, V. N. Synthesis. 1980, 1-26

Dansyl derivatives:

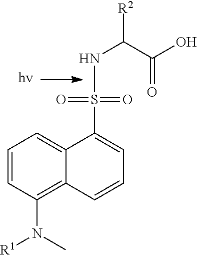

For more details see Rajasekharan Pillai, V. N. Synthesis. 1980, 1-26

Coumarin derivatives

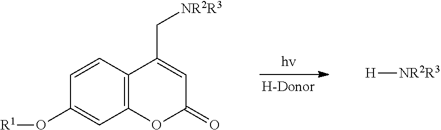

For more details see R. O. Schoenleber, B. Giese. Synlett 2003, 501-504

R¹ and R² can be any molecule or chemical entity (CE) such as those exemplified herein above under section A (acylation reactions), respectively. Moreover, R¹ and R² can be either the target or a solid support, respectively. R³ can be e.g. H or OCH₃ independently of R¹ and R². If X is O then the product will be a carboxylic acid. If X is NH the product will be a carboxamide One specific example is the PC Spacer Phosphoramidite (Glen research catalog #10-4913-90) which can be introduced in an oligonucleotide during synthesis and cleaved by subjecting the sample in water to UV light (−300-350 nm) for 30 seconds to 1 minute.

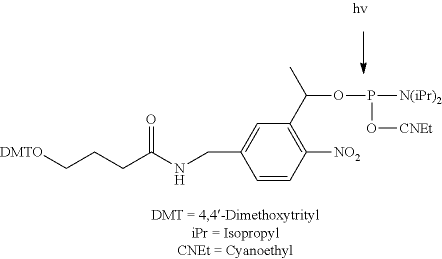

The above PC spacer phosphoamidite is suitable incorporated in a library of complexes at a position between the indentifier and the potential drug candidate. The spacer can be cleaved according to the following reaction.

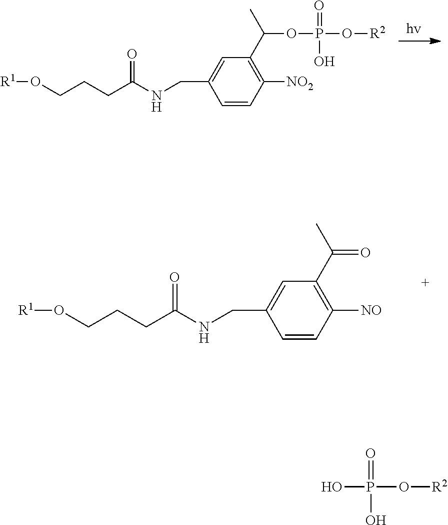

R¹ and R² can be any molecule or chemical entity (CE) such as those exemplified herein above under section A (acylation reactions). Moreover, R¹ and R² can be either the target or a solid support, respectively. In a preferred aspect R² is an oligonucleotide identifier and the R¹ is the molecule. When the linker is cleaved a phosphate group is generated allowing for further biological reactions. As an example, the phosphate group can be positioned in the 5′end of an oligonucleotide allowing for an enzymatic ligation process to take place.

### Examples of Linkers Cleavable by Chemical Agents:

Ester linkers can be cleaved by nucleophilic attack using e.g. hydroxide ions. In practice this can be accomplished by subjecting the target-ligand complex to a base for a short period.

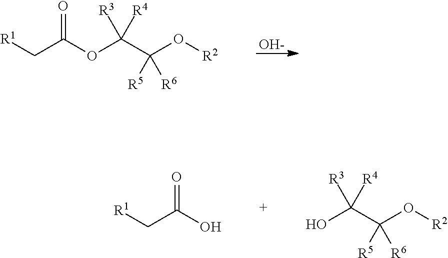

R¹ and R² can be the either of be the potential drug candidate or the identifier, respectively. R⁴⁻⁶ can be any of the following: H, CN, F, NO₂, SO₂NR₂.

Disulfide linkers can efficiently be cleaved/reduced by Tris (2-carboxyethyl) phosphine (TCEP). TCEP selectively and completely reduces even the most stable water-soluble alkyl disulfides over a wide pH range. These reductions frequently required less than 5 minutes at room temperature. TCEP is a non-volatile and odorless reductant and unlike most other reducing agents, it is resistant to air oxidation. Trialkylphosphines such as TCEP are stable in aqueous solution, selectively reduce disulfide bonds, and are essentially unreactive toward other functional groups commonly found in proteins.

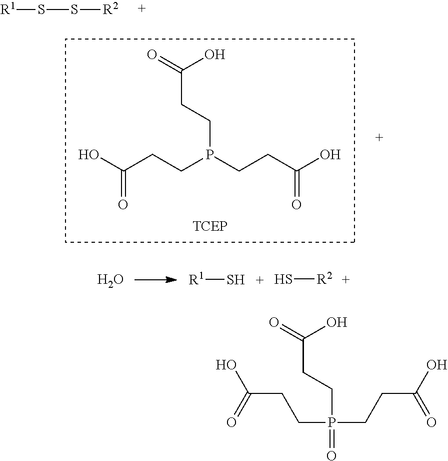

More details on the reduction of disulfide bonds can be found in Kirley, T. L. (1989), Reduction and fluorescent labeling of cyst(e)ine-containing proteins for subsequent structural analysis, Anal. Biochem. 180, 231 and Levison, M. E., et al. (1969), Reduction of biological substances by water-soluble phosphines: Gamma-globulin. Experentia 25, 126-127.

### Linkers Cleavable by Enzymes

The linker connecting the potential drug candidate with the identifier or the solid support and the target can include a peptide region that allows a specific cleavage using a protease. This is a well-known strategy in molecular biology. Site-specific proteases and their cognate target amino acid sequences are often used to remove the fusion protein tags that facilitate enhanced expression, solubility, secretion or purification of the fusion protein.

Various proteases can be used to accomplish a specific cleavage. The specificity is especially important when the cleavage site is presented together with other sequences such as for example the fusion proteins. Various conditions have been optimized in order to enhance the cleavage efficiency and control the specificity. These conditions are available and know in the art.

Enterokinase is one example of an enzyme (serine protease) that cut a specific amino acid sequence. Enterokinase recognition site is Asp-Asp-Asp-Asp-Lys, and it cleaves C-terminally of Lys. Purified recombinant Enterokinase is commercially available and is highly active over wide ranges in pH (pH 4.5-9.5) and temperature (4-45° C.).

The nuclear inclusion protease from tobacco etch virus (TEV) is another commercially available and well-characterized proteases that can be used to cut at a specific amino acid sequence. TEV protease cleaves the sequence Glu-Asn-Leu-Tyr-Phe-Gln-Gly/Ser between Gln-Gly or Gln-Ser with high specificity.

Another well-known protease is thrombin that specifically cleaves the sequence Leu-Val-Pro-Arg-Gly-Ser between Arg-Gly. Thrombin has also been used for cleavage of recombinant fusion proteins. Other sequences can also be used for thrombin cleavage; these sequences are more or less specific and more or less efficiently cleaved by thrombin. Thrombin is a highly active protease and various reaction conditions are known to the public.

Activated coagulation factor FX (FXa) is also known to be a specific and useful protease. This enzyme cleaves C-terminal of Arg at the sequence Ile-Glu-Gly-Arg. FXa is frequently used to cut between fusion proteins when producing proteins with recombinant technology. Other recognition sequences can also be used for FXa.

Other types of proteolytic enzymes can also be used that recognize specific amino acid sequences. In addition, proteolytic enzymes that cleave amino acid sequences in an unspecific manner can also be used if only the linker contains an amino acid sequence in the complex molecule.

Other type of molecules such as ribozymes, catalytically active antibodies, or lipases can also be used. The only prerequisite is that the catalytically active molecule can cleave the specific structure used as the linker, or as a part of the linker, that connects the encoding region and the displayed molecule or, in the alternative the solid support and the target. Also, a variety of endonucleases are available that recognize and cleave a double stranded nucleic acid having a specific sequence of nucleotides.

Tags

It is to be understood that when libraries of molecules linked to biologic entities are used, the ‘tag’ refers to the biological entity such as a phage particle, a yeast cell or a prokaryotic cell even though the actual information which encodes the molecule resides in genetic material such as a genome within the biologic entity. For example, a peptide displayed by a phage particle is physically linked to the phage particle and encoded by the phage genome residing within (surrounded by) the phage particle.

In preferred embodiments, molecules of the present invention are not tagged. For example, a collection of molecules may be provided in which each molecule has a unique weight and where the structure corresponding to each unique weight is known. Thus, by determining the weight of any molecule in the collection, the structure of the molecule is determined. In other preferred embodiments, molecules of the present invention are not tagged but they are positionally encoded. For example, a collection of molecules with known structures may be provided such that each molecule is placed in a unique location. Thus, by determining the location of any molecule in the collection, the structure of the molecule is determined. In yet other preferred embodiments, molecules of the present invention are tagged. Tags may be used to determine the synthetic history and structure of a molecule. In preferred embodiments, tags are chosen from; organic tags, such as amino acids, such as 13C-labeled amino acids; peptides, such as flourescently labeled peptide; proteins, such as antibodies or enzymes, saccharides, lipids, polynucleotides, such as PNA, LNA, RNA, or DNA, such as flouroscently labeled DNA, DNA folded in two-dimensional or 3-dimensional patterns, DNA nanostructures, encapsulated polynucleotides, such as encapsulated PNA, encapsulated LNA, encapsulated RNA, encapsulated DNA.

Other preferred tags are chosen from; polynucleotides or encapsulated polynucleotides comprising nucleotides selected from the group consisting of deoxyribonucleic acids (DNA), ribonucleic acids (RNA), peptide nucleic acids (PNA), locked nucleic acids (LNA), and morpholinos sequences, including any analog or derivative thereof. In another preferred embodiment, the tags employed in the methods of the present invention preferably comprise or essentially consist of nucleotides selected from the group consisting of DNA, RNA, PNA, LNA and morpholinos sequence, including any analog or derivative thereof, and anti-tags preferably comprise or essentially consist of nucleotides selected from the group consisting of DNA, RNA, PNA, LNA and morpholinos sequences, including any analog or derivative thereof. The nucleic acids useful in connection with the present invention include, but is not limited to, nucleic acids which can be linked together in a sequence of nucleotides, i.e. an oligonucleotide. However, in one embodiment and in order to prevent ligation of anti-tags (an anti-tag is an entity such as peptide, a protein, a polynucleotide which are capable of hybridizing to a tag), end-positioned nucleic acids of anti-tags do not contain a reactive group, such as a 5′-P or a 3′-OH reactive group, capable of being linked by e.g. an enzyme comprising ligase activity. The priming site of the display oligonucleotide preferably comprises a 3′-OH or 5′-phosphate group, or functional derivatives of such groups, capable of being linked by an enzyme comprising ligase activity. Each nucleotide monomer is normally composed of two parts, namely a nucleobase moiety, and a backbone. The back bone may in some cases be subdivided into a sugar moiety and an internucleoside linker. The nucleobase moiety can be selected among naturally occurring nucleobases as well as non-naturally occurring nucleobases. Thus, “nucleobase” includes not only known purine and pyrimidine hetero-cycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³-C⁶)-alkynylcytosine, 5-fluorouracil, 5-bromouracil, pseudoisocytosine, 2-hydroxy-5-methyl-4-triazolopyridine, isocytosine, isoguanine, inosine and the “non-natural” nucleobases described in U.S. Pat. No. 5,432,272. The term “nucleobase” is intended to cover these examples as well as analogues and tautomers thereof. Especially interesting nucleobases are adenine, guanine, thymine, cytosine, 5-methylcytosine, and uracil, which are considered as the naturally occurring nucleobases. Examples of suitable specific pairs of nucleobases are shown below:

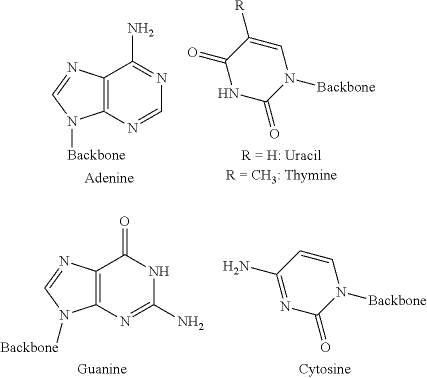

### Synthetic Base Pairs

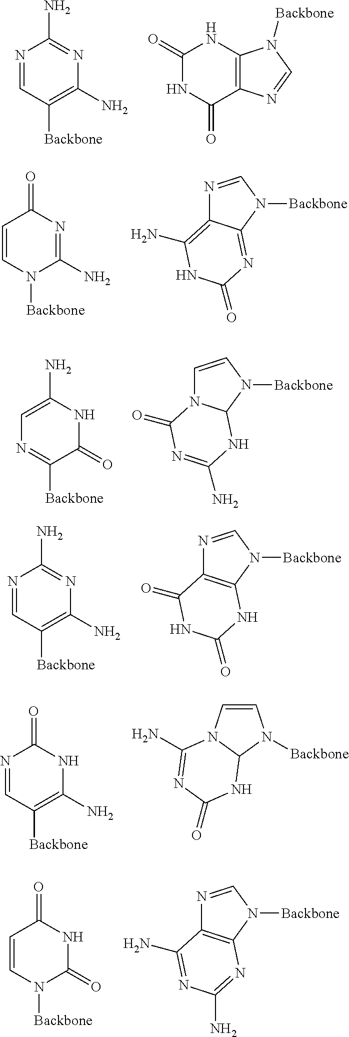

Synthetic purine bases pairing with natural pyrimidines

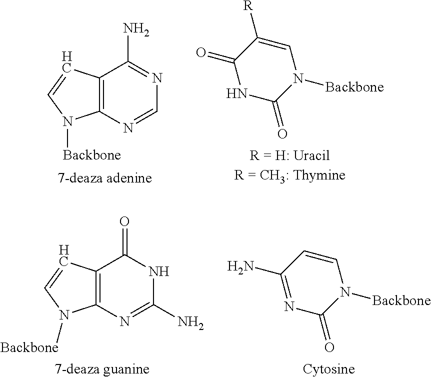

Suitable examples of backbone units are shown below (B denotes a nucleobase):

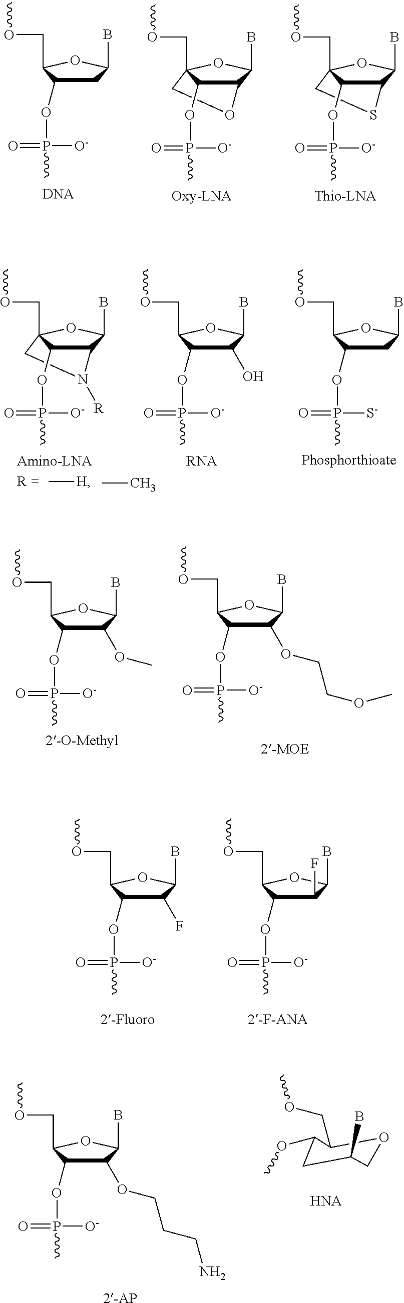

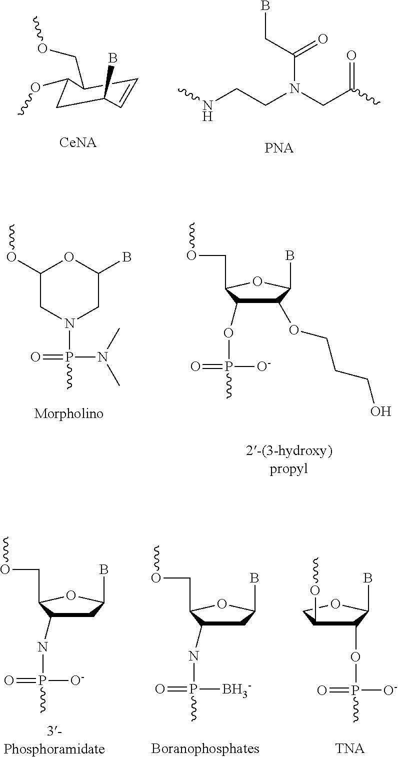

The sugar moiety of the backbone is suitably a pentose, but can be the appropriate part of an PNA or a six-member ring. Suitable examples of possible pentoses include ribose, 2′-deoxyribose, 2′-O-methyl-ribose, 2′-flour-ribose, and 2′-4′-O-methylene-ribose (LNA). Suitably the nucleobase is attached to the 1′ position of the pentose entity. An internucleoside linker connects the 3′ end of preceding monomer to a 5′ end of a succeeding monomer when the sugar moiety of the backbone is a pentose, like ribose or 2-deoxyribose. The internucleoside linkage can be the natural occurring phospodiester linkage or a derivative thereof. Examples of such derivatives include phosphorothioate, methylphosphonate, phosphoramidate, phosphotriester, and phosphodithioate. Furthermore, the internucleoside linker can be any of a number of non-phosphorous-containing linkers known in the art.

Preferred nucleic acid monomers include naturally occurring nucleosides forming part of the DNA as well as the RNA family connected through phosphodiester linkages. The members of the DNA family include deoxyadenosine, deoxyguanosine, deoxythymidine, and deoxycytidine. The members of the RNA family include adenosine, guanosine, uridine, cytidine, and inosine.

Other preferred tags may be chosen from; polymers, such as linear polymers, such as polyhalogenated phenoxyalkyl derivatives, mono-amides of iminodiacetic acid, coding system tags, such as haloaromatic units or secondary amine binary coding, linear polymers comprising a nucleobase, branched polymers, norganic tags, such as quantum dots, radio-frequency identification tags, metal tags, such as nanobarcodes, such as those produced by Nanoplex Technologies, Inc. Metallic nanoparticles, Chromium coated glass, Particles, such as nanorods, microspheres, Nanoparticles, such as silica nanoparticles, Bead arrays, Rare earth-doped glass bars, Dye molecules attached to silver or gold nanoparticles, Gold nanoparticles, such as gold particles with dyes attached, or ceramic plates with a two-dimensional, laser-etched bar codes.

Suitable tags are described in Li, Y., Cu, Y. T. H., and Luo, D., 2005. Multiplexed detection of pathogen DNA with DNA based fluorescence nanobarcodes. Nat. Biotechnol 23: 885-889.
Van, H., LaSean, T H., Feng, L., and Reif, H., 2003 Directed nucleation assembly of DNA tile complexes for barcode-patterned lattices. Proc Natl Acad Sci USA 100: 8 103-8 108.
Mandecki, W, Pappas, M, Kogan, N, Wang, Z, Zamlynny, B. 2002 Light-powered microtransponders for high multiple-level analyses of nucleic acids. ACS Symposium Series 8 15, 57-fJ9.
Storhoff, J. I., Lucas, A. D., GarimeUa, v., Bao, Y P., and MUller, U. R., 2004a Homogeneous detection of unamplified genomic DNA sequences based on colorimetric scatter of gold nanoparticle probes. Nat Biotechnol. 22: 883 887.
Galitonov, G. S., Birtwell, S. W., and Zheludcv, N.J., 2006 High capacity tagging using nanostructured diffraction barcodes. Optics Express 14: 1382-1387.
Finkel, N. H., Lou, X., Wang, C, and He, L., 2004 Barcoding the microworld.
Allal Chem 76: 352A-359A. Nicewamer-Pena, S. R., Freeman, R. G., Reiss, B. D., He, L., Pena, 0.1., Walton, 1.0., Cromer, R., Keating, C D., and Natan, M.1., 200 I Submicrometer metallic barcodes. Science 294: 137-141.
Fulton, R. J., McDade, R. L., Smith, P. L., Kienker, L J., and Keuman, J. R., Jr, 1997 Advanced multiplexed analysis with the FlowMetrix system. Ciin Chem 43: 1749-1756.
Bhalgat, M. K., Haugland, R. P., Pollack, 1.S., Swan, S., and Haugland, R. P., 1998 Green- and red fluorescent nanospheres for the detection of cell surface receptors by flow cytometry. J Immunol Methods 219: 57-68.
Wang, L. and Tan, W., 2006 Multicolor FRET silica nanoparticles by single wavelength excitation. Nano Lett 6: 84-88.
Gunderson, K. L., et al., 2004 DeCOding randomly ordered DNA arrays. Genome Res 14: 870-877.
Dejneka, 2003 Rare earth-doped glass microbarcodes. Proc Natl Acad Sci USA 100: 389-393.
Nie. S. and Emory, S. R. 1997 Probing single molecules and single nanoparticles by surface enhanced Raman scattering. Science 275: 1102-1106.
Lu, Y., Liu, G. L., Kim, J., Mejia, Y. X and Lee, L. P., 2005 Nanophotonic crescent moon structures with sharp edge for ultrasensitive biomolecular detection by local electromagnetic field enhancement effect. Nano Left 5: 119-124.

In preferred embodiments, molecules of the present invention are tagged with a thiol, an amine, an imine, an amino acid, polyamino acid, a peptide, a protein, a nucleotide, a ribonucleotide, a polynucleotide. In other preferred embodiments, molecules tagged with a polynucleotide tag, such as molecules described in US2006099592 (A1), EP1533385 (A1), WO2005003778 (A2), US2006121470 (A1), US2006099589 (A1), US2003100007 (A1), WO2005058479 (A3), WO2007053358 (A3), WO2008016547 (A3), WO2006048025 (A1), US2009239768 (A1), WO2009105657 (A1), WO2008054600 (A2), WO2006130669 (A2), WO2007016488 (A2), US2007154899 (A1), WO2006047791 (A2), WO0023458 (A1), WO2005090566 (A2), US2008193983 (A1), US2006269920 (A1), US2009264300 (A1), US2010184611 (A1), WO2009077173 (A2), US2005142583 (A1), WO2007008226 (A2), WO2006135654 (A2), WO2006138560 (A2), WO2006138666 (A2), WO2007011722 (A2), US2009149347 (A1), WO2010094036.

In preferred embodiments, molecules of the present invention can interact with a material. In other preferred embodiments, tags do not have high affinity for a surface or material which is used during the process of identifying molecules with desired characteristics. In other preferred embodiments, tags interact efficiently with a surface or material used during the process of identifying molecules with desired characteristics.

In preferred embodiments, tags comprise fragments chosen from; natural amino acids, non-natural amino acids, natural nucleosides, non-natural nucleosides. In preferred embodiments, tag fragments are linked by one or more bonds chosen from an amidhe bond, a single bond, a phosphodiester, a phosphothiate. In preferred embodiments, a tag comprises a number of tag fragments such as from 1-10, 10-100, 100-1000, 1000-10000 tag fragments.

### Tag Hybridization

In preferred embodiments, tags have the capability of hybridizing to themselves or to other entities. Such tags may for example be; a nucleotide which hybridizes partly or fully to itself, a nucleotide which hybridizes partly or fully to another nucleotide, a nucleotide which is linked to a molecule via a linker, where said nucleotide hybridizes partly or fully to itself, a nucleotide which is linked to a molecule via a linker, where said nucleotide hybridizes partly or fully to another nucleotide.

### Multi-Functional Tags

In one embodiment the tag is multi-functional. For example, a tag of the present invention may be monofunctional, bi-functional, tri-functional, etc. In a preferred embodiment, tags of the present invention are mono-functional because they only encode a molecule linked to said tag. In other preferred embodiments, tags are bi-functional because they encode a molecule linked to said tags and they have the ability to bind covalently or noncovalently with high affinity to a surface or material. In yet other preferred embodiments, tags are tri-functional because they encode a molecule, bind covalently or noncovalently with high affinity to a first surface or material, and furthermore, bind covalently or noncovalently with high affinity to a second surface or material, such as an antibody or a biotin-binding protein.

Multiple tags

In a preferred embodiment, a molecule is linked to multiple tags, such as a molecules linked to 2 tags. In another preferred embodiment, multiple molecules are linked to a single tag, such as a single tag linked to 2 molecules, 3 molecules or more molecules. In another preferred embodiment, multiple molecules are linked to multiple tags, such as 2 molecules linked to 2 tags, 3 molecules linked to 3 tags, or more molecules linked to more tags.

### Step B. Providing a Suitable Medium

In step (B) of the present invention a suitable medium is provided. A preferred medium according to the present invention is an organic solvent, e.g. when performing screenings where the material used is not efficiently dispersed in a non-organic medium, e.g. as is the case for carbon nanotubes or graphene. In preferred embodiments, the organic solvent is not miscible with water. For example, to perform screening of a phage display library in an organic solvent where the phage is preferable coated in a water-based layer the use of a water-immiscible organic solvent is preferred.

### Water-Miscible Solvents

In other preferred embodiments, the organic solvent is miscible with water. For example, to perform screening of a phage display library in an organic solvent, use of a water-miscible organic solvent will allow the solvent to efficiently solvate the phage particle without the formation of a water-based layer around the phage particle. Another preferred medium is water. Preferably, the ionic strength and other characteristics of a water-based medium are adjusted by adding salts etc. Also preferably, the pH of the medium is adjusted with one or more pH-buffering components. Preferred pH ranges are described below. In preferred embodiments, a step requires that a medium component such as an organic solvent is evaporated. Therefore, in preferred embodiments, the solvent has a boiling point below 100 degrees celcius, such as below 50 degrees celcius. In other preferred embodiments, the boiling point is preferably above 100 degrees celcius, such as above 200 degrees celcius, to allow selection under conditions where the medium is in a liquid state.

### Fluid/Soluble Polymers

An important aspect of the present invention is the use of soluble polymers in the screening process to mimic the end-environment in which the identified ligands will be used. For example, if ligands which can bind effectively to carbon nanotubes in a nylon matrix are sought, it is preferable to have nylon-mimicking entities such as soluble short nylon polymers present in the screening medium during screening. In other preferred embodiments, a soluble polymer medium comprises one or more repeat units. For example, if a molecule capable of binding efficiently to a carbon nanotube in the presence of polyethylene is sought, it is preferable that the medium comprises monomers or oligomers of a polyethylene repeat unit; such as X—(CH2-CH2-)n-X, where n=1 to 100, and where X=—CH3. In preferred embodiments, repeat units are substituted by one or more entities chosen from, but not limited to, —OH, —COOH or —NH2, for example to increase the solubility of said repeat units in a polar solvent. Such repeat units which mimic the parent polymer (in this case polyethylene) and may act as decoys to remove polymer-binding molecules, and thus ensure that identified molecules are capable of binding the material/surface (here CNT) in a polymer (polyethylene) environment.

In preferred embodiments, the number of repeat units of a polymer is chosen so that the polymer is soluble/fluid at a relevant temperature, such as at a temperature measured in degrees celcius of more than 0, such as more than 10, such as more than 20, such as more than 30, such as more than 40, such as more than 50, such as more than 60, such as more than 70, such as more than 80, such as more than 90. In other embodiments, said temperature measured in degrees celcius is preferably less than 100, such as less than 90, such as less than 80, such as less than 70, such as less than 60, such as less than 50, such as less than 40, such as less than 30, such as less than 20, such as less than 10.

In preferred embodiments fluid/soluble polymers are chosen from fluid forms, such as short chain forms, of the following polymers; a copolymer, a high density polyethylene (HDPE), a linear low density polyehtylene (LLDPE), a low density polyethylene (LDPE), a nylon polymer molecule, a polyacrylate, a polyamide (PA), a polycarbonate (PC), a polyethylene (PE), a polymer, a polyolefine, a polypropylene (PP) molecule, a polystyrene (PS), an acrylonitrile butadiene styrene polymer (ABS), an epoxy molecule, an organic polymer, a polyethylene terephtalate (PET), a polyvinylchloride (PVS), a styrene acrylonitrile copolymer, a styrene butadiene latex, an unsaturated polyester (UPR), a bis-maleimide (BMI), a polymeric cyanate ester, a A6 nylon, a polyarylether-etherketone (PEEK), a polyethylenimine (PEI), a poly-ether-ketone-ketone (PEKK), a poly(methyl methacrylate) (PMMA), a polyphenylene sulfide (PPS), a biopolymer, a block copolymer, a conductive polymer, a crosslinked polyethylene, a flouroplastic, a high density polypropylene, a low density polypropylene, a medium density polyethylene, a medium density polypropylene, a polyacrylonitrile, a polycaprolactone molecule, a polychloroprene, a polychlorotrifluoroethylene, a polyester molecule, a polyimide, a polylactic acid, a polyphenol, a polysulphone, a polytetrafluoroethylene, a polyurea, a polyurethane, a polyvinyl, a polyvinyl chloride (pVC), a silicone, an elastomer, an inorganic polymer, an ultra-high-molecular-weight polyethylene, a melamin resin, a neoprene, a superlinear polyethylene, poly(ethylene-vinyl acetate) (PEVA), polyamide, and polyoxymethylene (POM).

### Monomers/Repeat Units of Polymers

In preferred embodiment, the medium comprises one or more polymers, e.g in the form of short polymers, such as for example polymers with a distribution of lengths where the average length is short enough such that the polymers are soluble at the concentration an temperature used during screening.

For example, said repeat unit may be (—CH2-CH2-), i.e. the repeat unit of polyethylene ((—CH2-CH2-)ₙ). As another example, said repeat unit may be —(O)C—C6H4-C(O)O—CH2-CH2-O—), i.e. the repeat unit of Polyethylene terephthalate. Polymerization can generally be describes as a process of linking monomers into a covalently bonded linear og branched chain. During the polymerization process, some chemical groups may be lost from each monomer. This is the case, for example, in the polymerization of PET polyester. The monomers are terephthalic acid (HOOC—C6H4-COOH) and ethylene glycol (HO—CH2-CH2-OH) but the repeat unit is —OC—C6H4-COO—CH2-CH2-O—, which corresponds to the combination of the two monomers with the loss of two water molecules. The distinct piece of each monomer that is incorporated into the polymer is known as a repeat unit or monomer residue. As another example, the repetitive unit of polyethylene ((—CH2-CH2-)n) is (—CH2-CH2-) and the reactive unit may be an ethylene unit (H2C═CH2) which can react to form polyethylene (—CH2-CH2-)n.

Repeat units may optionally be substituted, e.g. by one or more substituent(s) chosen from —OH, —COOH, —NH2, —CHO.

Polymer repeat units suitable for use as components of the medium according to the present invention may be chosen from monomers of; styrene, ethylene, vinylchloride, tetrafluoroethylene, imide, ethylene terephthalate, chloroprene, olefin, arylate, arylether, arylsulfone, butylene, carbonate, ester, etherimide, estercarbonate, esterketone, ketone, ethersulfone, propylene In preferred embodiments, the medium comprises one or more polymers comprising 1-1000 repeat units. For example, the medium comprises a single repeat unit or reactive unit such as caprolactone or a mixture of adipic acid and hexamethylene diamine.

Polymers suitable for use as components of the medium according to the present invention and polymers which contain repeat units suitable for use as components of the medium according to the present invention may be selected from; Polyethylenes: Polyethylene (PE), Low density polyethylene (LDPE), High density polyethylene (HDPE), Linear low density polyethylene (LLDPE), Crosslinked polyethylene (XLPE), Ultra High Molecular Weight Polyethylene (UHMWPE). Other Polyolefins: Polypropylene (PP), Biaxially-oriented polypropylene, Polybutylene (PB), Polyisobutene (PIB) Polyacrylates: Polymethyl methacrylate (PMMA), polymethyl acrylate (PMA), hydroxyethyl methacrylate (HEMA), Sodium polyacrylate. Polystyrenes: Polystyrene (PS), High impact polystyrene (HIPS), Extruded polystyrene (XPS), Expanded Polystyrene, Polyesters: Polyethylene terephthalate (PET). Polysulfones: Polysulfone (PSU), Polyarylsulfone (PAS), Polyethersulfone PES, Polyphenylsulfone (PPS). Polyamides: Polyamide (PA), polyphthalamide (PPA), Bismaleimide (BMI), urea formaldehyde (UF) Polyurethanes: Polyurethane (PU), Polyisocyanurate (PIR). Chloropolymers: Polyvinyl chloride (PVC), Polyvinylidene dichloride (PVDC). (Chloro)fluoropolymers: Fluoropolymer (FE), Polytetrafluoroethylene (PTFE), Polyvinylidene difluoride (PVDF), Polychlorotrifluoroethlyene (PCTFE), Ethylene chlorotrifluoroethlyene (ECTFE). Other Homopolymers: Polycarbonate (PC), Polylactic acid (PLA), Polyacrylamide (PAM), Polyetheretherketone (PEEK). Other Copolymers: Acrylonitrile butadiene styrene (ABS), Polybutadiene acrylonitrile (PBAN). Polymers suitable for use according to the present invention may also be selected from; BMI Bismaleimide, Cellulose acetate, Cellulose acetate butyrate, Cellulose acetate propionate, Cellulose propionate, Cellulosics, cyamelide, ECTFE Ethylene chlorotrifluoroethlyene, ETFE Ethylene TetrafluoroEthylene, Ethyl cellulose, FEP Fluorinated Ethylene Propylene, HDPE High density polyethylene, HEMA hydroxyethyl methacrylate, HEMA hydroxyethyl methacrylate, LDPE Low density polyethylene, LLDPE Linear low density polyethylene, Methyl methacrylate, PA Polyamide, PAM polyacrylamide, PAM polyacrylamide, PAN polyacrylonitrile, PAN polyacrylonitrile, PAS Polyarylsulfone, PB Polybutylene, PBAN polybutadiene acrylonitrile, PBAN polybutadiene acrylonitrile, PBT Polybutylene terephthalate, PCT Type, PCTA Type, PCTFE Polychlorotrifluoroethylene (Teflon), PCTG Type, PE Polyethylene, PEBA Polyether Block Amide elastomer, PES Polyethersulfone, PET Polyethylene terephthalate, PETG Type, PEX Crosslinked high density polyethylene, PFA Perfluoroalkoxyethylene, PIB Polyisobutene, PMA polymethyl acrylate, PMA polymethyl acrylate, PMMA polymethyl methacrylate, PMMA polymethyl methacrylate, Polyacrylate, Polyaryletherketones, polyurea, Polyvinyl, Polyvinyl acetate, Polyvinyl alcohol, Polyvinyl carbazole, Polyvinyl chloride, PP Polypropylene, PPA polyphthalamide, PPS Polyphenylsulfone, PSU Polysulfone, PTFE Polytetrafluoroethylene, PVDF Polyvinylidene fluoride, PVF Polyvinyl fluoride, Sodium polyacrylate, UF urea formaldehyde, UHMWPE Ultra high molecular weight polyethylene, XLPE Crosslinked Polyethylene, Alkyd, Allyl Diglycol Carbonate Monomer, Bismaleimide (BMI), DAIP (Diallyl Isophthalate), DAP (Diallyl Phthalate), DCPD Resin, Epoxy, Furan, Melamine, Melamine/Phenolic, Phenolic, Polybutandiene, Polyester (Unsaturated), Bulk Molding Compounds (BMC), Gel Coats, Granular Molding Compounds (GMC), Resins, Sheet Molding Compounds (SMC), Thick Molding Compounds (TMC), Polyimide (Thermosetting), Silicone, Urea, Urethane Chemicals, Vinyl Ester, Molding Compounds, ABS, acrylonitrile-butadiene-styrene copolymer; ACMA, acrylonitrile-methyl acrylate copolymer; AIBN, 2,2-azobisisobutyronitrile; ATRP, atom transfer radical polymerization; CNT, carbon nanotube; CPP, chlorinated polypropylene; DWCNT, double-walled carbon nanotube; EMMA, ethyl-methyl methacrylate copolymer; EPDM, ethylene-propylene-diene rubber; EVA, ethylene-vinyl acetate copolymer; EVOH, ethylene-vinyl alcohol copolymer; HDPE, high-density polyethylene; HMW, high molecular weight; LDPE, low density polyethylene; LMW, low molecular weight; MA, maleic anhydride; MBMA, methyl-butyl methacrylate copolymer; MDPE, medium density polyethylene; MEMA, methyl-ethyl methacrylate copolymer; MPTS, methacryloxypropyltrimethoxysilane; MWCNT, multi-walled carbon nanotube; NMP, nitroxide-mediated polymerization; NMR, nuclear magnetic resonance; P3HT, poly(3-hexylthiophene); P3OT, poly(3-octylthiophene); PA, poly-acetylene; PAA, poly(acrylic acid); PABS, poly(m-aminobenzene sulfonic acid); PAM, polyacrylamide; PAMAM, poly(amidoamine); PAN, polyacrylonitrile; PANI, polyaniline; Parmax, poly(benzoyl-1,4-phenylene)-co-(1,3-phenylene); PBA, polybutyl acrylate; PtBA, poly(tert-butyl acrylate); PBMA, poly(butyl methacrylate); PBO, poly(phenylenebenzobisoxazole); PBT, poly(butyl terephthalate); PC, polycarbonate; PCL, polycaprolactone; PDEAEMA, poly[2-(diethylamino)ethyl methacrylate]; PDI, polydispersity index; PDMEMA, poly[2-(dimethylamino)ethyl methacrylate]; PDMS, polydimethylsiloxane; PDPA, polydiphenylamime; PE, polyethylene; PEG, polyethyleneglycol; PEI, polyethyleneimine; PEMA, poly(ethyl methacrylate); PEO, polyethyleneoxide; PET, poly(ethyl terephthalate); PETI, phenylethynyl-terminated imide; PGMA, poly(glycerol monomethacrylate); PHEMA, poly(2-hydroxyethyl methacrylate); PHET, poly[3-(2-hydroxyethyl)-2,5-thienylene]; PHPMA, poly[N-(2-hydroxypropyl)methacrylamide]; PI, polyimide; PIMA, poly(imidazolium methacrylate); PLLA, poly(1-lactic acid); PLLA-g-AA, poly(1-lactic acid) grafted with poly(acrylic acid) chains; PMDMAS, poly[3-(N-(3-methacrylamidopropyl)-N,N-dimethyl)ammoniopropanatesulfonate]; PMMA, poly(methyl methacrylate); PMMAHEMA, poly[(methyl methacrylate)-co-(2-hydroxyethyl methacrylate)]; PmPV, poly(m-phenylenevinylene-co-2,5-dioctoxy-p-phenylenevinylene); PNIPAAm, poly(N-isopropylacrylamide); PP, polypropylene; PPE, poly(p-phenylene ethynylene); PPEI-EI, poly(propionylethylenimine-co-ethylimine); PPS, poly(phenylene sulfide); PPY, polypyrrole; PS, polystyrene; PSS, poly(sodium 4-styrenesulfonate); PSV, poly(styrene-co-p-(4-(4-vinylphenyl)-3-oxobutanol)); PTH, polythiophene; PU, polyurethane; PVA, poly(vinyl alcohol); PVAc, poly(vinyl acetate); PVAc-VA, poly(vinyl acetate-co-vinyl alcohol); PVC, poly(vinyl chloride); PVDF, poly(vinylidene fluoride); PVK, poly(N-vinyl carbazole); PVKV, poly(N-vinyl carbazole-co-p-(4-(4-vinylphenyl)-3-oxobutanol)); PVP, polyvinylpyrrolidone; P2VP, poly(2-vinylpyridine); P4VP, poly(4-vinylpyridine); SAN, styrene-acrylonitrile copolymer; SBA, styrene-butyl acrylate copolymer; SBBS, styrene-butadiene-butylene-styrene copolymer; SBR, styrene-butadiene rubber; SCMS, styrene-p-chloromethylstyrene copolymer; SE, silicone elastomer; SIBS, poly(styrene-b-isobutylene-b-styrene); SMA, styrene maleic anhydride copolymer; STM, scanning tunneling microscopy; SWCNT, single-walled carbon nanotube; TDI, toluene diisocyanate; TEMPO, 2,2,6,6-tetramethylpiperidinyl-1-oxy; THF, tetrahydrofuran; UHMWPE, ultra high molecular weight polyethylene; WBPU, waterborne polyurethane.

In preferred embodiments, a repeat unit is chosen from; mono-styrene, mono-ethylene, mono-vinylchloride, mono-tetrafluoroethylene, mono-imide, mono-ethylene terephthalate, mono-chloroprene, mono-olefin, mono-arylate, mono-arylether, mono-arylsulfone, mono-butylene, mono-carbonate, mono-ester, mono-etherimide, mono-estercarbonate, mono-esterketone, mono-ketone, mono-ethersulfone, mono-propylene,

Di-styrene, di-Ethylene, di-Vinylchloride, di-tetrafluoroethylene, di-imide, di-ethylene terephthalate, di-chloroprene, di-olefin, di-arylate, di-arylether, di-arylsulfone, di-butylene, di-carbonate, di-ester, di-etherimide, di-estercarbonate, di-esterketone, di-ketone, di-ethersulfone, di-propylene, Tri-styrene, tri-Ethylene, tri-Vinylchloride, tri-tetrafluoroethylene, tri-imide, tri-ethylene terephthalate, tri-chloroprene, tri-olefin, tri-arylate, tri-arylether, tri-arylsulfone, tri-butylene, tri-carbonate, tri-ester, tri-etherimide, tri-estercarbonate, tri-esterketone, tri-ketone, tri-ethersulfone, tri-propylene, Tetra-styrene, tetra-Ethylene, tetra-Vinylchloride, tetra-tetrafluoroethylene, tetra-imide, tetra-ethylene terephthalate, tetra-chloroprene, tetra-olefin, tetra-arylate, tetra-arylether, tetra-arylsulfone, tetra-butylene, tetra-carbonate, tetra-ester, tetra-etherimide, tetra-estercarbonate, tetra-esterketone, tetra-ketone, tetra-ethersulfone, tetra-propylene. The average chain length of each polymer is to be adjusted to ensure in the medium of choice.

In other preferred embodiments, repeat units have one of the following formulas B1 and B2:

R((—R)ₙ)ₘ B1:

R₁—R₂— . . . —Rₙ B2:

### Where

n is a number from 0 to 1e6
m is a number from 1 to 10
, and each R is individually selected from the group comprising; a non-covalent bond, a covalent bond, a single bond, a double bond, a triple bond, —CH2-, —C(O)—, —NH—, —O—, —S—, —SO2-, —CH2CH2-, —C(O)CH2-, —CH2C(O)—, —NHCH2-, —CH2NH—, —OCH2-, —CH2O—, —SCH2-, —CH2S—, —SO2CH2-, —CH2SO2-, —NHC(O)—, —C(O)NH—, —NHSO2-, —SO2NH—, —CH2CH2CH2-, —CH2CH2C(O)—, —CH2CH2NH—, —CH2CH2O—, —CH2CH2S—, —CH2CH2SO2-, —CH2C(O)CH2-, —CH2NHCH2-, —CH2OCH2-, —CH2SCH2-, —CH2SO2CH2-, —C(O)CH2CH2-, -NHCH2CH2-, -OCH2CH2-, -SCH2CH2-, -SO2CH2CH2-, —CH2C(O)NH—, —CH2SO2NH—, —CH2NHC(O)—, —CH2NHSO2-, —C(O)NHCH2-, -SO2NHCH2-, —NHC(O)CH2-, -NHSO2CH2-, —NHC(O)NH—; cycloalkyl, aryl, heterocyclyl, and heteroaryl, azetidinyl, cyclopropanyl, cyclobutanyl, cyclopentanyl, cyclohexanyl, cyclohexanylcyclobutyl, cyclohexanylcyclopropyl, cyclohexylcyclohexyl, phenylcyclobutyl, phenylcyclobutyl, phenylcyclohexyl, phenoxycyclobutyl, phenoxycyclopentyl, phenoxycyclohexyl, benzylcyclobutyl, benzylcyclobutyl, benzylcyclohexyl, phenylaminocyclobutyl, phenylaminocyclobutyl, phenylaminocyclohexyl, 7-azabicyclo[4.2.0]octa-1,3,5-trienyl, 2,3-dihydro-1H-indolyl, 1,2,3,4-tetrahydroquinolinyl, 2,3-dihydro-1H-isoindolyl, 1,2,3,4-tetrahydroisoquinolinyl, phenylazetidinyl, phenylpyrrolidinyl, phenylpiperidinyl, phenylazetidinyl, phenylazetidinonyl, phenylpyrrolidinonyl, phenylpiperidinonyl, phenoxyazetidinyl, phenoxypyrrolidinyl, phenoxypiperidinyl, phenoxyazetidinyl, phenoxypyrrolidinyl, phenoxypiperidinyl, phenoxypiperidinyl, phenoxyazetidinonyl, phenoxypyrrolidinonyl, phenoxypiperidinonyl, benzylazetidinyl, benzylpyrrolidinyl, benzylpiperidinyl, benzylazetidinonyl, benzylpyrrolidinonyl, benzylpiperidinonyl, phenylaminoazetidinyl, phenylaminopyrrolidinyl, phenylaminopiperidinyl, phenylaminoazetidinyl, phenylaminoazetidinonyl, phenylaminopyrrolidinonyl, phenylaminopiperidinonyl, phenyl, phenylphenyl, benzylphenyl, phenoxyphenyl, phenylaminophenyl, phenylsulfanylphenyl, phenylcarbonylphenyl, naphtyl, phenalenyl, anthracenyl, phenylnaphtyl, 5-phenylnaphthalen-2-yl, phenylfuranyl, phenylpyrrolyl, phenylthiophenyl, phenylisoxazolyl, phenyloxazolyl, phenyloxadiazolyl, benzylisoxazolyl, benzyloxazolyl, benzyloxadiazolyl, thiazolyl, phenylthiazolyl, imidazolylthiazolyl, pyrazinylthiazolyl, phenylthiadiazolyl, [1,3]thiazolo[5,4-b]pyridinyl, [1,3]oxazolo[5,4-b]pyridinyl, 3H-imidazo[4,5-b]pyridinyl, [1,3]thiazolo[5,4-c]pyridinyl, [1,3]oxazolo[5,4-c]pyridinyl, 3H-imidazo[4,5-c]pyridinyl, [1,3]thiazolo[4,5-c]pyridinyl, [1,3]oxazolo[4,5-c]pyridinyl, 1H-imidazo[4,5-c]pyridinyl, [1,3]thiazolo[5,4-c]pyridazinyl, [1,3]oxazolo[5,4-c]pyridazinyl, 7H-imidazo[4,5-c]pyridazinyl, [1,3]thiazolo[5,4-d]pyrimidinyl, [1,3]oxazolo[5,4-d]pyrimidinyl, 9H-purinyl, [1,3]thiazolo[4,5-d]pyridazinyl, [1,3]oxazolo[4,5-d]pyridazinyl, 1H-imidazo[4,5-d]pyridazinyl, [1,3]thiazolo[5,4-d][1,2,3]triazinyl, [1,3]oxazolo[5,4-d][1,2,3]triazinyl, 7H-imidazo[4,5-d][1,2,3]triazinyl, phenylpyrazolyl, phenyltriazolyl, phenyltetrazolyl, benzylpyrazolyl, benzyltriazolyl, benzyltetrazolyl, naphatalenylcyclopropanyl, naphtalenylmethylcyclobutanyl, naphtalenylaminocyclopentanyl, napthalenyloxyazetidinyl, naphtalenylcarbonylpyrrolidinyl, naphatalenylpiperidinyl, naphtalenylmethylazetidinonyl, naphtalenylaminopyrrolidinonyl, napthalenyloxypiperidinonyl, naphtalenylcarbonylpyrazolyl, naphatalenyltriazolyl, naphtalenylmethyltetrazolyl, naphtalenylaminofuranyl, napthalenyloxypyrrolyl, naphtalenylcarbonylthienyl, and naphatalenyloxazolyl.

Each R may individually and optionally be substituted by one or more substituents preferably selected from the group consisting of H, methyl, hydroxyl, —NH2, —CN, —F, —Cl, —Br, —CH2OH, —O—CH3, —CH2F, —CHF2, —CF3, —CH2CI1-CH2CH2OH1-0-CH2CH3, —SO2, —NO2, ethyl, —CH2CF3, —CF2CF3, propyl, isopropyl, 2-methylpropyl, and tert-butyl, cyclopropyl, cyclobutyl, cyclopentyl, cyclohexyl, azetidinyl, pyrrolidinyl, piperidinyl, tetrahydrofuranyl, tetrahydro-2H-pyranyl, isoxazolidinyl, morpholinyl, oxazolidinyl, oxazinanylyl, tetrahydrothiophene, tetrahydro-2H-thiopyranyl, isothiazolidinyl, thiomorpholinyl, thiazolidinyl, thiazinanylyl, pyrazolidinyl, imidazolidinyl, hexahydropyrimidinyl, pyranyl, dihydropyridinyl, dihydropyrrole, piperazinyl, azetidinonyl, azepanylyl, oxazetidinyl, diazetidinyl, oxazepanylyl, diazepanylyl, pyrrolidinonyl, piperidinonyl, azepanylonyl, thioxoazetidinyl, phenyl, cyclopentadienyl, pyrrolyl, furanyl, isoxazolyl, oxazolyl, thienyl, thiazolyl, isothiazolyl, imidazolyl, oxadiazolyl, thiadiazolyl, oxathiazolyl, pyrimidinyl, triazinyl, tetrazine, pyrazine, pyridazine, pyrazolyl, triazolyl, tetrazolyl, imidazolyl, 2,4,5,6-tetrahydrocyclopenta[c]pyrrolyl, -dihydro-cyclopentatolfuranyl, 5,6-dihydro-4H-cyclopenta[c]thiophenyl, 4,5,6,7-tetrahydro-2H-isoindolyl, 4,5,6,7-tetrahydroisobenzofuranyl, 4,5,6,7-tetrahydrobenzo[c]thiophenyl, 2,4-dihydrocyclopenta[c]pyrrolyl, 4H-cyclopenta[c]furanyl, 4H-cyclopenta[c]thiophenyl, 2H-isoindolyl, isobenzofuranyl, and benzo[c]thiophenyl.

In some embodiments, polymers are added to the medium as a composition of polymer chains with different numbers of repeat units and thus different chain lengths. Such compositions have short chains and longer chains and may be characterized by the minimum, the maximum and the average chain length. Polymer compositions with different chain length distributions may be obtained by varying polymerization conditions such as polymerization temperatures and/or by terminating growing polymer chains by adding terminating entities to the polymerization mixture, e.g. by adding monoamines or monoacids in the case of nylon polymerization.

For example the polymerization of adipic acid (HOOC—(CH₂)₄—COOH) and hexamethylene diamine (NH₂—(CH₂)₄)—NH₂) may be terminated by adding terminating entities such as monoacids, e.g. acetic acid (CH3COOH), or by adding monoamines, e.g. propylamine CH₃CH₂CH₂NH₂.

Thus by varying the concentration of timing of addition of terminating entities, polymer compositions with different chain length distributions and average chain lengths may be obtained.

In cases where a polymer compositions of high solubility in a medium is required, a low average chain length measured in the number of monomers is the most important characteristic of the polymer composition. Thus, dependent on context, the average number of linked monomers is preferably lower than 10000, such as lower than 2000, such as lower than 1000, such as lower than 500, such as lower than 200, such as lower than 100, such as lower than 50, such as lower than 10, such as lower than 1. In cases where a polymer compositions of low solubility in a medium is required, a high average chain length measured in the number of monomers is the most important characteristic of the polymer composition. Thus, dependent on context, the average number of linked monomers is preferably higher than 1, such as higher than 10, such as higher than 50, such as higher than 100, such as higher than 200, such as higher than 500, such as higher than 1000, such as higher than 2000, such as higher than 10000.

Repeated monomers for said high or low solubility polymer compositions are preferably chosen from monomers of the following polymers; a nylon, a polyacrylate, a polyamide (PA), a polycarbonate (PC), a polyethylene (PE), a polymer, a polyolefine, a polypropylene (PP) molecule, a polystyrene (PS), an acrylonitrile butadiene styrene polymer (ABS), an epoxy-based polymer, an organic polymer, a polyethylene terephtalate (PET), a polyvinylchloride (PVC), a styrene acrylonitrile copolymer, a styrene butadiene latex, an unsaturated polyester (UPR), a bis-maleimide (BMI), a polymeric cyanate ester, A6 nylon, a polyarylether-etherketone (PEEK), a polyethylenimine (PEI), a poly-ether-ketone-ketone (PEKK), a poly(methyl methacrylate) (PMMA), a polyphenylene sulfide (PPS), poly(ethylvinyl alcohol)(EVA). Preferred repeat units/monomers are; ethylene carbonate, propylene carbonate, caprolactone, caprolactam, butyl vinyl ether, cis-chlorobutadiene, chloroprene, 2-chloro-1,3-butadiene, ethylene adipate, ethylene oxide/oxirane, ethyl vinyl ether, trans-Isoprene, propylene oxide, vinyl butyral, vinyl acetal, or poly(butylene succinate). Other preferred monomers which can be included in a medium to mimic a polymer are cyclic entities such as; lactams, such as β-propiolactam, γ-butyrolactam, δ-valerolactam, and ε-caprolactam; lactones, such as α-acetolactone, β-propiolactone, γ-butyrolactone, and δ-valerolactone; lactols, such as

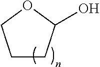

where n=1 to 5; cyclic ethers, such as oxacyclopentane, oxacyclohexane, oxacycloheptane.

The medium may be adjusted by adding components such as solvents to ensure that monomers/repeat units of polymers or short polymers stay in solution. The following table lists preferred repeat units and corresponding preferred solvents:

| Repeating Unit | Solvents |
|---|---|
| Acetylene | Isopropylamine, aniline |
| Acrylamide | Morpholine, water |
| Acrylate esters | Aromatic hydrocarbons, chlorinated hydrocarbons, THF, esters, |
| | ketones |
| Acrylic acid | Alcohols, water, dilute aqueous alkali |
| Acrylonitrile | Phenylenediamines, ethylene carbonate, sulfuric acid |
| Alkyl vinyl ethers | Benzene, halogenated hydrocarbons, methyl ethyl ketone |
| Amic acids | DMF, DMSO, tetramethylurea |
| Aryl sulfonates | DMF |
| Butadiene | Hydrocarbons, THF, higher ketones |
| ε-Caprolactam (Nylon 6) | m-Cresol, chlorophenol, formic acid |
| Cellulose | Trifluoroacetic acid, aqueous solutions of cupriethylenediamine |
| Cellulose ethers | Aqueous alkali |
| Cellulose triacetate | Methylene chloride, THF, ethylene carbonate |
| Chloroprene | Benzene, chlorinated hydrocarbons, pyridine |
| Ethylene | Above 80° C.: halogenated hydrocarbons, higher aliphatic esters and |
| | ketones |
| Ethylene phthalamide | Sulfuric acid |
| Ethylene terephthalate | Trichloroacetaldehyde hydrate, phenol, chlorophenol |
| Ethylene oxide | Chloroform, alcohols, esters |
| Formaldehyde | At elevated temperature: phenol, aniline, ethylene carbonate |
| Hexamethylene adipate | Trichloroethanol, phenols, sulfuric acid |
| (Nylon 6/6) | |
| Isobutene | Chlorinated hydrocarbons, THF, aliphatic ethers |
| Isoprene | Hydrocarbons, THF, higher ketones |
| Lactic acid | Chloroform, dioxane |
| Maleic anhydride | Dioxane, ethers, ketones |
| Methacrylate esters | Benzene, methylene chloride, methyl ethyl ketone |
| Methacrylic acid | Alcohols, water, dilute aqueous sodium hydroxide |
| 1,4-Phenylene ethylene | Biphenyl, phenyl ether |
| Phenylene sulfone | Methylene chloride, DMSO |
| Phenyl glycidyl ether | Xylene (hot), 1,2-dichlorobenzene (hot) |
| Propylene | Above 80° C.: halogenated hydrocarbons, higher aliphatic esters and |
| | ketones |
| Propylene oxide | Benzene, chloroform, ethanol |
| Pyromellitimides | m-Cresol, conc. sulfuric acid |
| Siloxanes | Aromatic and chlorinated hydrocarbons, esters |
| Styrene | Benzene, chlorinated aliphatic hydrocarbons, methyl ethyl ketone, |
| | ethyl acetate |
| Tetrafluoroethylene | Perfluorokerosene (350° C.) |
| Thiophenylene | Biphenyl, dichlorobiphenyl |
| Ureas | Phenol, m-cresol, formic acid |
| Urethanes | Phenol, m-cresol, formic acid |
| Vinyl acetal | Benzene, chloroform, THF |
| Vinyl acetate | Toluene, chloroform, methanol |
| Vinyl alcohol | Glycols (hot), water, piperazine |
| Vinyl butyryl | Methylene chloride, alcohols, ketones |
| Vinyl chloride | THF, methyl ethyl ketone |
| Vinyl carbazole | Chloroform, chlorobenzene, dioxane |
| Vinylidene chloride | THF(hot), trichloroethane |
| Vinylidene fluoride | Cyclohexanone, ethylene carbonate |
| N-Vinyl pyrrolidone | Chloroform, ethanol, pyridine |

(Retrieved from https://www.sigmaaldrich.com/content/dam/sigma-aldrich/docs/Aldrich/General_Information/polymer_solutions.pdf; A more extensive selection is found in “Polymer Handbook”, Eds. Brandrup, J.; Immergut, E. H.; Grulke, E. A., 4th Edition, John Wiley, New York, 1999, VII/497-535. Aldrich Catalog Number Z41, 247-3.)

Preferred Characteristics of Components of the Medium

### Solubility

In many cases it is preferred that components of the medium has a high solubility such that a uniform medium can be ensured. For example, to ensure a uniform mix of water and an organic solvent, it is preferred that the solvent has a high solubility in water, i.e. a high miscibility with water. In cases where the presence of a high amount of a given component in a solvent is the most important characteristic, a high solubility is preferred. Thus, depending on the context, the solubility as measured in moles per liter is preferably higher than 10⁻¹⁸, such as higher than 10⁻¹⁷, such as higher than 10⁻¹⁶, such as higher than 10⁻¹⁵, such as higher than 10⁻¹⁴, such as higher than 10⁻¹³, such as higher than 10⁻¹², such as higher than 10⁻¹¹, such as higher than 10⁻¹⁰, such as higher than 10⁻⁹, such as higher than 10⁻⁸, such as higher than 10⁻⁷, such as higher than 10⁻⁶, such as higher than 10⁻⁵, such as higher than 10⁻⁴, such as higher than 10⁻³, such as higher than 10⁻², such as higher than 10.

In other cases it is preferred that a component of the medium precipitates. Thus, a low solubility of that component is the most important characteristic. Thus, depending on the context, the solubility as measured in moles per liter is preferably lower than 10, such as lower than 100, such as lower than 10⁻², such as lower than 10⁻³, such as lower than 10⁻⁴, such as lower than 10⁻⁵, such as lower than 10⁻⁶, such as lower than 10⁻⁷, such as lower than 10⁻⁸, such as lower than 10⁻⁹, such as lower than 10⁻¹⁰, such as lower than 10⁻¹¹, such as lower than 10⁻¹², such as lower than 10⁻¹³, such as lower than 10⁻¹⁴, such as lower than 10⁻¹⁵, such as lower than 10⁻¹⁶, such as lower than 10⁻¹⁷, such as lower than 10⁻¹⁸, such as lower than 10⁻¹⁹.

### Melting Point

For any characteristic of a component of the medium mentioned above, and in each characteristic's entire range, a further characteristic of importance is the melting point of the component of the medium. To identify ligands which retain significant affinity towards a material at a high temperature, it is important to perform screening such that every medium component is liquid, i.e. not a solid, at a high temperature. Thus, depending on context, the melting temperature of a medium component as measured in Kelvin is preferably higher than 0, such as higher than 50, such as higher than 200, such as higher than 350, such as higher than 500, such as higher than 650, such as higher than 800, such as higher than 950, such as higher than 1100, such as higher than 1250, such as higher than 1400, such as higher than 1550, such as higher than 1700, such as higher than 1850, such as higher than 2000, such as higher than 2150, such as higher than 2300, such as higher than 2450, such as higher than 2600, such as higher than 2750. To identify ligands which retain significant affinity towards a material at a low temperature, it is important to perform screening such that every medium component is liquid, i.e, not a solid, at a low temperature. Thus, depending on context, the melting temperature of a medium component as measured in Kelvin is preferably lower than 2900, such as lower than 2750, such as lower than 2600, such as lower than 2450, such as lower than 2300, such as lower than 2150, such as lower than 2000, such as lower than 1850, such as lower than 1700, such as lower than 1550, such as lower than 1400, such as lower than 1250, such as lower than 1100, such as lower than 950, such as lower than 800, such as lower than 650, such as lower than 500, such as lower than 350, such as lower than 200, such as lower than 50.

### Boiling Point

For any characteristic of a component of the medium mentioned above, and in each characteristic's entire range, a further characteristic of importance is the boiling point of the component of the medium. To identify ligands which retain significant affinity towards a material at a high temperature, it is important to perform screening such that every medium component is liquid, i.e. not a gas, at a high temperature. Thus, depending on context, the boiling temperature of a medium component as measured in Kelvin is preferably higher than 0, such as higher than 50, such as higher than 200, such as higher than 350, such as higher than 500, such as higher than 650, such as higher than 800, such as higher than 950, such as higher than 1100, such as higher than 1250, such as higher than 1400, such as higher than 1550, such as higher than 1700, such as higher than 1850, such as higher than 2000, such as higher than 2150, such as higher than 2300, such as higher than 2450, such as higher than 2600, such as higher than 2750. To identify ligands which retain significant affinity towards a material at a low temperature, it is important to perform screening such that every medium component is liquid, i.e. not a gas, at a low temperature. Thus, depending on context, the boiling temperature of a medium component as measured in Kelvin is preferably lower than 2900, such as lower than 2750, such as lower than 2600, such as lower than 2450, such as lower than 2300, such as lower than 2150, such as lower than 2000, such as lower than 1850, such as lower than 1700, such as lower than 1550, such as lower than 1400, such as lower than 1250, such as lower than 1100, such as lower than 950, such as lower than 800, such as lower than 650, such as lower than 500, such as lower than 350, such as lower than 200, such as lower than 50.

### Dielectric Constant

For any characteristic of a component of the medium mentioned above, and in each characteristic's entire range, a further characteristic of importance is the dielectric constant of the component of the medium. To efficiently identify ligands from molecule libraries it is important that molecules are soluble in the medium used. Thus, to identify hydrophobic ligands a low dielectric constant is the most important characteristic of components of the medium. Thus, depending on the context, the dielectric constant of the medium is preferably lower than 250000, such as lower than 200000, such as lower than 50000, such as lower than 20000, such as lower than 1000, such as lower than 500, such as lower than 200, such as lower than 100, such as lower than 80, such as lower than 40, such as lower than 30, such as lower than 20, such as lower than 15, such as lower than 10, such as lower than 8, such as lower than 5, such as lower than 4, such as lower than 3, such as lower than 2, such as 1. To identify hydrophilic ligands a high dielectric constant is the most important characteristic of components of the medium. Thus, depending on the context, the dielectric constant of the medium is preferably higher than 1, such as higher than 2, such as higher than 3, such as higher than 4, such as higher than 5, such as higher than 8, such as higher than 10, such as higher than 15, such as higher than 20, such as higher than 30, such as higher than 40, such as higher than 80, such as higher than 100, such as higher than 200, such as higher than 500, such as higher than 1000, such as higher than 20000, such as higher than 50000, such as higher than 200000, such as higher than 250000.

cLogP, Calculated Logarithm of the Octanol-Water Partition Coefficient

For any characteristic of a component of the medium mentioned above, and in each characteristic's entire range, a further characteristic of importance is the cLogP of the component of the medium. In cases where hydrophopic ligands are sought, e.g. for binding to a hydrophobic surface, a high cLogP is the most important characteristic of medium components. Thus, depending on context, the cLogP of components of the medium is preferably higher than −100, such as higher than −90, such as higher than −80, such as higher than −70, such as higher than −60, such as higher than −50, such as higher than −40, such as higher than −30, such as higher than −20, such as higher than −10, such as higher than 0, such as higher than 10, such as higher than 20, such as higher than 30, such as higher than 40, such as higher than 50, such as higher than 60, such as higher than 70, such as higher than 80, such as higher than 90. In cases where hydrophilic ligands are sought, e.g. for binding to a hydrophilic surface, a low cLogP is the most important characteristic of the medium components. Thus, depending on context, the cLogP of medium components is preferably lower than 90, such as lower than 80, such as lower than 70, such as lower than 60, such as lower than 50, such as lower than 40, such as lower than 30, such as lower than 20, such as lower than 10, such as lower than 0, such as lower than −10, such as lower than −20, such as lower than −30, such as lower than −40, such as lower than −50, such as lower than −60, such as lower than −70, such as lower than −80, such as lower than −90, such as lower than −100.

### pH Buffering Components

In preferred embodiments, the medium comprises one or more pH buffering components.

Buffers suitable for use according to the present invention may comprise one or more of the following components, which in the following are listed with—within square brackets—pKA values at 25 degrees celcius followed by semicolon followed by pH buffering range: ACES [6.78; 6.1-7.5], acetate [4.76; 3.6-5.6], ADA [6.59; 6.0-7.2], ammonium hydroxide [9.25; 8.8-9.9], AMP (2-amino-2-methyl-1-propanol) [9.69; 8.7-10.4], AMPD (2-amino-2-methyl-1,3-propanediol) [8.80; 7.8-9.7], AMPSO [9.00; 8.3-9.7], BES [7.09; 6.4-7.8], BICINE [8.26; 7.6-9.0], bis-tris [6.46; 5.8-7.2], BIS-TRIS propane [6.80, 9.00; 6.3-9.5], borate [9.23, 12.74, 13.80; 8.5-10.2], CABS [10.70; 10.0-11.4], cacodylate [6.27; 5.0-7.4], CAPS [10.40; 9.7-11.1], CAPSO [9.60; 8.9-10.3], carbonate (pK1) [6.35; 6.0-8.0], carbonate (pK2) [10.33; 9.5-11.1], CHES [9.50; 8.6-10.0], citrate (pK1) [3.13; 2.2-6.5], citrate (pK2) [4.76; 3.0-6.2], citrate (pK3) [6.40; 5.5-7.2], DIPSO [7.52; 7.0-8.2], EPPS, HEPPS [8.00; 7.6-8.6], ethanolamine [9.50; 6.0-12.0], formate [3.75; 3.0-4.5], glycine (pK1) [2.35; 2.2-3.6], glycine (pK2) [9.78; 8.8-10.6], glycylglycine (pK1) [3.14; 2.5-3.8], glycylglycine (pK2) [8.25; 7.5-8.9], HEPBS [8.30; 7.6-9.0], HEPES [7.48; 6.8-8.2], HEPPSO [7.85; 7.1-8.5], histidine [1.70, 6.04, 9.09; 5.5-7.4], hydrazine [8.10; 7.5-10.0], imidazole [6.95; 6.2-7.8], malate (pK1) [3.40; 2.7-4.2], malate (pK2) [5.13; 4.0-6.0], maleate (pK1) [1.97; 1.2-2.6], maleate (pK2) [6.24; 5.5-7.2], MES [6.10; 5.5-6.7], methylamine [10.66; 9.5-11.5], MOBS [7.60; 6.9-8.3], MOPS [7.14; 6.5-7.9], MOPSO [6.87; 6.2-7.6], phosphate (pK1) [2.15; 1.7-2.9], phosphate (pK2) [7.20; 5.8-8.0], phosphate (pK3) [12.33;], piperazine (pK1) [5.33; 5.0-6.0], piperazine (pK2) [9.73; 9.5-9.8], piperidine [11.12; 10.5-12.0], PIPES [6.76; 6.1-7.5], POPSO [7.78; 7.2-8.5], propionate [4.87; 3.8-5.6], pyridine [5.23; 4.9-5.9], pyrophosphate [0.91, 2.10, 6.70, 9.32; 7.0-9.0], succinate (pK1) [4.21; 3.2-5.2], succinate (pK2) [5.64; 5.5-6.5], TABS [8.90; 8.2-9.6], TAPS [8.40; 7.7-9.1], TAPSO [7.61; 7.0-8.2], taurine (AES) [9.06; 8.4-9.6], TES [7.40; 6.8-8.2], tricine [8.05; 7.4-8.8], triethanolamine (TEA) [7.76; 7.0-8.3], Trizma (tris) [8.06; 7.5-9.0]

pH

In cases where ligands are sought which can bind a material under acidic conditions, a low pH is the most important characteristic of the medium. Thus, depending on context, the pH is the medium is preferably adjusted by addition of one or more acids or pH-buffering components such that the pH is lower than 15, such as lower than 14, such as lower than 13, such as lower than 12, such as lower than 11, such as lower than 10, such as lower than 9, such as lower than 8, such as lower than 7, such as lower than 6, such as lower than 5, such as lower than 4, such as lower than 3, such as lower than 2, such as lower than 1, such as lower than 0, such as lower than −1. In cases where ligands are sought which can bind a material under basic conditions, a high pH is the most important characteristic of the medium. Thus, depending on context, the pH is the medium is preferably adjusted by addition of one or more bases or pH-buffering components such that the pH is higher than −1, such as higher than 0, such as higher than 1, such as higher than 2, such as higher than 3, such as higher than 4, such as higher than 5, such as higher than 6, such as higher than 7, such as higher than 8, such as higher than 9, such as higher than 10, such as higher than 11, such as higher than 12, such as higher than 13, such as higher than 14, such as higher than 15. For example, the pH of commercially available concentrated HCl solution (37% by mass) has pH of about −1.1, while saturated NaOH solution has pH of about 15.0. (Dickerson, R. E.; Gray, H. B.; Darensbourg, M. Y. Chemical Principles, 4th ed.; Benjamin Cummings: Menlo Park, Calif., 1984.)

### Solvents

In preferred embodiments, the medium comprises one or more solvents chosen from Water, Acetal (1,1-Diethoxyethane), Acetic acid, Acetone, Acetonitrile, Acetylacetone, Acrylonitrile, Adiponitrile, Allyl alcohol, Allylamine, 2-Aminoisobutanol, Benzal, Benzaldehyde, Benzene, Benzonitrile, Benzyl Chloride, Bromochloromethane, Bromoform (Tribromomethane), Butyl acetate, Butyl alcohol, sec-Butyl alcohol, tert-Butyl alcohol, Butylamine, tert-Butylamine, Butyl methyl ketone, p-tert-Butyltoluene, g-Butyrolactone, Caprolactam, Carbon disulfide, Carbon tetrachloride, 1-Chloro-1,1-difluoroethane, Chlorobenzene, Chloroform, Chloropentafluoroethane, Cumene (Isopropylbenzene), Cyclohexane, Cyclohexanol, Cyclohexanone, Cyclohexylamine, Cyclopentane, Cyclopentanone, p-Cymene, cis-Decalin, trans-Decalin, Diacetone alcohol, 1,2-Dibromoethane, Dibromofluoromethane, Dibromomethane, 1,2-Dibromotetrafluoroethane, Dibutylamine, o-Dichlorobenzene, 1,1-Dichloroethane, 1,2-Dichloroethane, 1,1-Dichloroethylene, cis-1,2-Dichloroethylene, trans-1,2-Dichloroethylene, Dichloroethyl ether, Dichloromethane, 1,2-Dichloropropane, 1,2-Dichlorotetrafluoroethane, Diethanolamine, Diethylamine, Diethyl, Diethylene glycol, Diethylene glycol dimethyl ether, Diethylene glycol monoethyl ether, Diethylene glycol monoethyl ether acetate, Diethylene glycol monomethyl ether, Diethylenetriamine, Diethyl ether, Diisobutyl ketone, Diisopropyl ether, N,N-Dimethylacetamide, Dimethylamine, Dimethyl disulfide, N,N-Dimethylformamide, Dimethyl sulfoxide, 1,4-Dioxane, 1,3-Dioxolane, Dipentene, Epichlorohydrin, Ethanolamine (Glycinol), Ethyl acetate, Ethyl acetoacetate, Ethyl alcohol, Ethylamine, Ethylbenzene, Ethyl bromide, Ethyl Chloride, Ethylene Carbonate, Ethylenediamine, Ethylene glycol, Ethylene glycol diethyl ether, Ethylene glycol dimethyl ether, Ethylene glycol monobutyl ether, Ethylene glycol monoethyl ether, Ethylene glycol ethyl ether acetate, Ethylene glycol monomethyl ether, Ethylene glycol momomethyl ether acetate, Ethyl formate, Furan, Furfural, Furfuryl alcohol, Glycerol, Heptane, 1-Heptanol, Hexane, 1-Hexanol (Caproyl alcohol), Hexylene glycol, Hexyl methyl ketone, Isobutyl acetate, Isobutyl alcohol, Isobutylamine, Isopentyl acetate, Isophorone, Isopropyl acetate, Isopropyl alcohol, Isoquinoline, d-Limonene (Citrene), 2,6-Lutidine, Mesitylene, Mesityl oxide, Methyl acetate, Methylal, Methyl alcohol, Methylamine, Methyl benzoate, Methylcyclohexane, Methyl ethyl ketone, N-Methylformamide, Methyl formate, Methyl iodide, Methyl isobutyl ketone, Methyl isopentyl ketone, 2-Methylpentane, 4-Methyl-2-pentanol, Methyl pentyl ketone, Methyl propyl ketone, N-Methyl-2-pyrrolidone, Morpholine, Nitrobenzene, Nitroethane, Nitromethane, 1-Nitropropane, 2-Nitropropane, Octane, 1-Octanol, Pentachloroethane, Pentamethylene glycol, Pentane, 1-Pentanol, Pentyl acetate, 2-Picoline, a-Pinene, b-Pinene, Piperidine, Propanenitrile, Propyl acetate, Propyl alcohol, Propylamine, Propylbenzene, Propylene glycol, Pseudocumene, Pyridine, Pyrrole, Pyrrolidine, 2-Pyrrolidone, Quinoline, Styrene, Sulfolane, a-Terpinene, 1,1,1,2-Tetrachloro-2,2-difluoroethane, 1,1,2,2-Tetrachloro-1,2-difluoroethane, 1,1,1,2-Tetrachloroethane, 1,1,2,2-Tetrachloroethane, Tetrachloroethylene, Tetraethylene glycol, Tetrahydrofuran, 1,2,3,4-Tetrahydronaphthalene, Tetrahydropyran, Tetramethylsilane, Toluene, o-Toluidine, Triacetin, Tributylamine, 1,1,1-Trichloroethane, 1,1,2-Trichloroethane, Trichloroethylene, Trichlorofluoromethane, 1,1,2-Trichlorotrifluoroethane, Triethanolamine, Triethylamine, Triethylene glycol, Triethyl phosphate, Trimethylamine, Trimethylene glycol, Trimethyl phosphate, Veratrole, o-Xylene, m-Xylene, p-Xylene. Preferred solvents are water-miscible solvents, e.g. acetaldehyde, acetic acid, acetone, acetonitrile, 1,2-Butanediol, 1,3-Butanediol, 1,4-Butanediol, 2-Butoxyethanol, butyric acid, diethanolamine, diethylenetriamine, dimethylformamide, dimethoxyethane, dimethyl sulfoxide, 1,4-Dioxane, ethanol, ethylamine, ethylene glycol, formic acid, furfuryl alcohol, glycerol, methanol, methyl diethanolamine, methyl isocyanide, 1-Propanol, 1,3-Propanediol, 1,5-Pentanediol, 2-Propanol, propanoic acid, propylene glycol, pyridine, tetrahydrofuran, triethylene glycol

### Molecular Weight (MW)

For any characteristic of a component of the medium mentioned above, and in each characteristic's entire range, a further characteristic of importance is the molecular weight of the component of the medium. In cases, where low evaporation is the most important characteristic of components of the medium, said components preferably have a high molecular weight. Thus, depending on the context, the MW as measured in Dalton is preferably greater than 10, such as greater than 100, such as greater than 10², such as greater than 10³, such as greater than 10⁴, such as greater than 10⁵, such as greater than 10⁶, such as greater than 10⁸, such as greater than 10⁹. In cases, where ease of evaporation is the most important characteristic of components of the medium, said components preferably have a low molecular weight. Thus, depending on the context, the MW as measured in Dalton is preferably lower than 10⁹, such as lower than 10⁸, such as lower than 10⁷, such as lower than 10⁶, such as lower than 10⁵, such as lower than 10⁴, such as lower than 10³, such as lower than 10², such as lower than 10. In cases where both evaporation and thus molecular weight must be balanced, the MW of components of the medium measured in Dalton is preferably; 10¹ to 10², or 10² to 10³, or 10³ to 10⁴, or 10⁴ to 10⁵, or 10⁵ to 10⁶, or 10⁶ to 10⁷, or 10⁷ to 10⁸, or 10⁸ to 10⁹.

### Hydrogen Bond Donors (HBD)

For any characteristic of a component of the medium mentioned above, and in each characteristic's entire range, a further characteristic of importance is the number of hydrogen bonds of the component of the medium. In cases where ligands with a high number of hydrogen bond acceptors are sought, a high number of HBD is the most important characteristic of the medium component, in order to efficiently solvate library molecules. Thus, depending on context, the number of HBD of library molecules is preferably more than 0, such as more than 1, such as more than 2, such as more than 3, such as more than 4, such as more than 5, such as more than 6, such as more than 7, such as more than 8, such as more than 9, such as more than 12, such as more than 15, such as more than 18, such as more than 21, such as more than 24, such as more than 27, such as more than 30, such as more than 33, such as more than 36, such as more than 39, such as more than 42, such as more than 45, such as more than 48, such as more than 51, such as more than 54, such as more than 57, such as more than 60, such as more than 63, such as more than 66, such as more than 69, such as more than 72, such as more than 75, such as more than 78, such as more than 81, such as more than 84, such as more than 87, such as more than 90, such as more than 93, such as more than 96, such as more than 99. In cases where ligands with a low number of hydrogen bond acceptors are sought, a low number of HBD is the most important characteristic of the medium component. Thus, depending on context, the number of HBD of a component of the medium is preferably more than 99, such as less than 96, such as less than 93, such as less than 90, such as less than 87, such as less than 84, such as less than 81, such as less than 78, such as less than 75, such as less than 72, such as less than 69, such as less than 66, such as less than 63, such as less than 60, such as less than 57, such as less than 54, such as less than 51, such as less than 48, such as less than 45, such as less than 42, such as less than 39, such as less than 36, such as less than 33, such as less than 30, such as less than 27, such as less than 24, such as less than 21, such as less than 18, such as less than 15, such as less than 12, such as less than 9, such as less than 8, such as less than 7, such as less than 6, such as less than 5, such as less than 4, such as less than 3, such as less than 2, such as less than 1.

### Hydrogen Bond Acceptors (HBA)

For any characteristic of a component of the medium mentioned above, and in each characteristic's entire range, a further characteristic of importance is the number of hydrogen bond acceptors of the component of the medium. In cases where ligands with a high number of hydrogen bond donors are sought, a high number of HBA is the most important characteristic of the component of the library to efficiently solvate library molecules. Thus, depending on context, the number of HBA is preferably more than 1, such as more than 2, such as more than 3, such as more than 4, such as more than 5, such as more than 6, such as more than 7, such as more than 8, such as more than 9, such as more than 11, such as more than 13, such as more than 15, such as more than 17, such as more than 19, such as more than 21, such as more than 23, such as more than 25, such as more than 27, such as more than 29, such as more than 34, such as more than 39, such as more than 44, such as more than 49, such as more than 54, such as more than 59, such as more than 64, such as more than 69, such as more than 74, such as more than 79. In cases where ligands with a low number of hydrogen bond donors are sought, a low number of HBAs is the most important characteristic of components of the medium. Thus, depending on context, the number of HBAs of library molecules is preferably less than 94, such as less than 89, such as less than 84, such as less than 79, such as less than 74, such as less than 69, such as less than 64, such as less than 59, such as less than 54, such as less than 49, such as less than 44, such as less than 39, such as less than 34, such as less than 29, such as less than 27, such as less than 25, such as less than 23, such as less than 21, such as less than 19, such as less than 17, such as less than 15, such as less than 13, such as less than 11, such as less than 9, such as less than 8, such as less than 7, such as less than 6, such as less than 5, such as less than 4

### Blocking Agent

In preferred embodiments, the medium comprises a blocking agent which serves to eliminate or reduce unwanted binding between library molecules and a surface or between tags and surface or between molecule and tag. Said blocking agents may be chosen from; bovine serum albumin, skimmed milk powder, e. coli extract, yeast extract, skimmed milk protein, CTAB, HTAB, OTAB, Oligonuclotides, polynucleotides, such as DNA, such as single-stranded DNA, Biomolecule, such as protein, inactivated phages, such as phages treated with subtilisin, e.g. as described in Schwind et al. J. Biochem. 210,431-436 (1992).

### Surfactant

In preferred embodiments, the medium comprises a surfactant, or a mixture of surfactants. For example, if carbon nanotubes are used as surface/material it is preferable that the medium comprises a surfactant to keep the nanotube in solution. Said surfactant or mixture of surfactants may be chosen from the following list where the supplier (Sigma) product number is optionally followed by CAS REGISTRY number in square brackets; A carbon nanotube detergent such as Dipalmitoylphosphatidylcholine (DPPC), dihexanoylphosphatidylcholine (DHPC), lysophosphatidylcholine (LPC), Tween-20, Triton X-100, sodium dodecyl sulfate (SDS). 1-Methyl-1′-tetradecyl-4,4′-bipyridinium dichloride; 1-Octanesulfonic acid sodium salt ˜98% [08380, 5324-84-5]; 1-Octanesulfonic acid sodium salt BioXtra [00133, 5324-84-5]; 1-Octyl-2-pyrrolidone purum, ≥98.0% (GC) [75072, 2687-94-7]; 1-Oleoyl-rac-glycerol ˜99% [M7765, 111-03-5]; 1-Oleoyl-rac-glycerol ≥99.0% (TLC) [49959, 111-03-5]; 1-Oleoyl-rac-glycerol technical, ˜40% (TLC) [49960, 111-03-5]; 2-Cyclohexylethyl β-D-maltoside ≥99.0% (TLC) [29395, 260804-65-7]; 3-(1-Pyridinio)-1-propanesulfonate 2 M solution [11102, 15471-17-7]; 3-(1-Pyridinio)-1-propanesulfonate purum, ≥97.0% (N) [82804, 15471-17-7]; 3-(4-tert-Butyl-1-pyridinio)-1-propanesulfonate ≥98.0% (HPLC) New [53487, 570412-84-9]; 3-(Benzyldimethylammonio)propanesulfonate BioXtra, ≥99.0% (HPCE) [17236, 81239-45-4]; 3-(Decyldimethylammonio)propanesulfonate inner salt [D4266, 15163-36-7]; 3-(N,N-Dimethylmyristylammonio)propanesulfonate [T0807, 14933-09-6]; 3-(N,N-Dimethylmyristylammonio)propanesulfonate ≥98.0% (T) [40772, 14933-09-6]; 3-(N,N-Dimethylmyristylammonio)propanesulfonate ≥99% (TLC) [T7763, 14933-09-6]; 3-(N,N-Dimethyloctadecylammonio)propanesulfonate purum, ≥99.0% (TLC) [41570, 13177-41-8]; 3-(N,N-Dimethyloctylammonio)propanesulfonate 500 mM solution [54185, 15178-76-4]; 3-(N,N-Dimethyloctylammonio)propanesulfonate inner salt [06626, 15178-76-4]; 3-(N,N-Dimethylpalmitylammonio)propanesulfonate [H6883, 2281-11-0]; 3-[N,N-Dimethyl(3-palmitoylaminopropyl)ammonio]-propanesulfonate [94508, 52562-29-5]; 4-Dodecylbenzenesulfonic acid mixture of isomers, ≥95% [44198, 121-65-3]; 4-Nonylphenoxypolyglycidyl ether suitable for photographic applications [N7521,]; 4-Nonylphenyl-polyethylene glycol [3853, 9016-45-9]; 5-Cyclohexylpentyl β-D-maltoside ≥98.0% (TLC) [96193, 250692-65-0]; 6-Cyclohexylhexyl β-D-maltoside ≥99.0% (TLC) [29396, 228579-27-9]; Alkyltrimethylammonium bromide ≥95% (TLC) [M7635,]; Amprolium hydrochloride [A0542, 137-88-2]; Amprolium hydrochloride VETRANAL®, analytical standard [31592, 137-88-2]; Antifoam 204 [A6426,]; Antifoam A Concentrate [A5633,]; Antifoam B Emulsion [A5757,]; Antifoam C Emulsion [A8011,]; Antifoam Y-30 Emulsion [A5758,]; ASB 14-4 ≥97.0% (TLC) New [77644, 122398-33-8]; ASB-14 [A1346, 216667-08-2]; ASB-14 ≥98.0% (HPCE) [53327, 216667-08-2]; ASB-C80 ≥95% (TLC) New [49845, 216667-49-1]; Benzalkonium chloride BioXtra [B6295, 63449-41-2]; Benzalkonium chloride [12063, 63449-41-2]; Benzalkonium chloride purum, ≥95.0% (T) [12060, 63449-41-2]; Benzethonium chloride ≥97% (titration), ≥99% (TLC) [B8879, 121-54-0]; Benzethonium chloride [70585, 121-54-0]; Benzethonium hydroxide solution ˜1.0 M in methanol (by HCl titration) [B2156, 498-77-1]; Benzyldimethyldodecylammonium chloride puriss., ≥99.0% (AT) [13380, 139-07-1]; Benzyldimethylhexadecylammonium chloride [B4136, 122-18-9]; Benzyldimethylhexadecylammonium chloride ≥97.0% (dried material, AT) [13400, 122-18-9]; Benzyldimethyltetradecylammonium chloride puriss., anhydrous, ≥99.0% (AT) [13401, 139-08-2]; Benzyldodecyldimethylammonium bromide [B5776, 1965463]; Benzyldodecyldimethylammonium bromide puriss., ≥99.0% (AT) [13373, 1965463]; Bile salts BioChemika, for microbiology [48305,]; Bis(polyethylene glycol bis[imidazoyl carbonyl]) [P9532,]; Brij™ 30 [P4391, 9002-92-0]; Brij™ 30 average Mn ˜362 [235989, 9002-92-0]; Brij™ 30 main component: tetraethylene glycol dodecyl ether [16001, 9002-92-0]; Brij™ 35 P main component: tricosaethylene glycol dodecyl ether [16005, 9002-92-0]; Brij™ 35 P solution ˜10% in H2O [16012, 9002-92-0]; Brij™ 52 main component: diethylene glycol hexadecyl ether [16002, 9004-95-9]; Brij™ 72 main component: diethylene glycol octadecyl ether [16006, 9005-00-9]; Brij™ 92V main component: diethylene glycol oleyl ether [16009, 9004-98-2]; Brij™ 98 average Mn ˜1,150 [436240, 9004-98-2]; Brij™ C10 main component: decaethylene glycol hexadecyl ether [16003, 9004-95-9]; Brij™ L23 solution 30% (w/v) [B4184, 9002-92-0]; Brij™ L23 suitable for Stein-Moore chromatography [P1254, 9002-92-0]; Brij™ O10 [P6136, 9004-98-2]; Brij™ O10 main component: decaethylene glycol oleyl ether [16011, 9004-98-2]; Brij™ S10 main component: decaethylene glycol octadecyl ether [16007, 9005-00-9]; Brij™ 58 average Mn ˜1124 [P5884, 9004-95-9]; C7BzO [C0856,]; Cetylpyridinium chloride [C0732, 6004-24-6]; CHAPS ≥98% (TLC) [C3023, 75621-03-3]; CHAPS ≥98.0% (TLC) [26680, 75621-03-3]; CHAPS 100 mM solution [19899, 75621-03-3]; CHAPS BioReagent, suitable for electrophoresis, ≥98% (TLC) [C9426, 75621-03-3]; CHAPS BioXtra, ≥98% (TLC) [C5070, 75621-03-3]; CHAPSO ≥90.0% (TLC) [26675, 82473-24-3]; CHAPSO ≥98% [C3649, 82473-24-3]; CHAPSO BioReagent, suitable for electrophoresis [C9551, 82473-24-3]; CHAPSO BioXtra [C4695, 82473-24-3]; Chenodeoxycholic acid ≥95% [C9377, 474-25-9]; Chenodeoxycholic acid diacetate methyl ester [C2025, 2616-71-9]; Cholic acid from ox or sheep bile, ≥98% [C1129, 81-25-4]; Choline p-toluenesulfonate salt ˜99% [C5787, 55357-38-5]; Cremophor® A25 ˜100% active ingredients basis [95921, 68439-49-6]; Cremophor® A6 ˜100% active ingredients basis [43058, 85941-44-2]; Cremophor® EL [27963, 61791-12-6]; Cremophor® EL pH-range 6.0-8.0 [C5135, 61791-12-6]; Cyclohexylmethyl 3-D-maltoside ≥99.0% (TLC) [29467, 260804-64-6]; DDMAB ≥97.0% [413, 15163-30-1]; Decaethylene glycol monododecyl ether [P9769, 9002-92-0]; Decyl 3-D-glucopyranoside ≥98% (GC) [D5394, 58846-77-8]; Decyl β-D-glucopyranoside ≥99.0% (TLC) [30710, 58846-77-8]; Decyl β-D-maltopyranoside ≥98% (GC) [D7658, 82494-09-5]; Decyl-3-D-1-thiomaltopyranoside ≥99.0% (TLC) [30727, 148565-56-4]; Decyl-β-D-maltoside 10 mM solution [7509, 82494-09-5]; Dehydrocholic acid ≥99.0% (T) [30830, 81-23-2]; Deoxycholic acid ≥99% (TLC and titration) [D2510, 83-44-3]; Deoxycholic acid BioXtra, ≥99% (TLC and titration) [D4297, 83-44-3]; DGEA ≥97% (TLC) [42355, 134580-64-6]; Dicyclohexyl sulfosuccinate sodium salt purum, ≥98.0% (TLC) [86141, 23386-52-9]; Diethylene glycol analytical standard [3128, 111-46-6]; Diethylene glycol BioUltra, ≥99.0% (GC) [93171, 111-46-6]; Diethylene glycol monodecyl ether ≥98.0% (GC) [32267, 23238-41-7]; Diethylene glycol monotetradecyl ether ≥99.0% (TLC) [32295, 56049-79-7]; Diethylene glycol puriss. p.a., ≥99.0% (GC), colorless [32160, 111-46-6]; Diethylene glycol purum, ≥98.0% (GC) [32170, 111-46-6]; Diethylene glycol ReagentPlus®, 99% [H26456, 111-46-6]; Digitonin [D141, 11024-24-1]; Digitonin ˜50% (TLC) [D5628, 11024-24-1]; Digitoxigenin [D9404, 143-62-4]; Dihexadecyl phosphate [D2631, 2197-63-9]; Dihexadecyl phosphate purum, ≥98.0% (TLC) [37170, 2197-63-9]; Dihexyl sulfosuccinate sodium salt solution technical, ˜80% in H2O [86146, 3006-15-3]; Dimethyldecylphosphine oxide ≥98.0% (GC) [40108, 2190-95-6]; Dimethyldioctadecylammonium bromide ≥98% (TLC) [D2779, 3700-67-2]; Dimethyldioctadecylammonium bromide purum, ≥98.0% (AT) [40165, 3700-67-2]; Dimethyldioctadecylammonium bromide Selectophore®, ≥99.0% (T) [40163, 3700-67-2]; Dimethylethylammoniumpropane sulfonate ≥97% (TLC) [D0195, 160255-06-1]; Docusate sodium meets USP testing specifications [D1685, 577-11-7]; Docusate sodium salt BioUltra, ≥99.0% (TLC) [86139, 577-11-7]; Docusate sodium salt BioXtra, ≥99% [D4422, 577-11-7]; Dodecylethyldimethylammonium bromide purum, ≥98.0% (AT) [44165, 68207-00-1]; Dodecyltrimethylammonium bromide ≥98% [D8638, 1119-94-4]; Dodecyltrimethylammonium bromide ≥98.0% (AT) [44240, 1119-94-4]; Dodecyltrimethylammonium bromide BioXtra, ˜99% [D5047, 1119-94-4]; Dodecyltrimethylammonium bromide for ion pair chromatography, ≥99.0% (AT) [44239, 1119-94-4]; Dodecyltrimethylammonium chloride puriss., ≥99.0% (AT) [44242, 112-00-5]; Dodecyltrimethylammonium chloride purum, ≥98.0% (AT) [17104, 112-00-5]; Dodecyltrimethylammonium chloride solution purum, ˜50% in isopropanol/water 4:1 (AT) [44241, 112-00-5]; EMPIGEN® BB detergent ˜35% active substance in H2O [45165, 66455-29-6]; Ethanesulfonic acid sodium salt monohydrate purum, ≥98.0% (T) [2430, 308103-56-2]; Ethylene glycol monodecyl ether ≥97.0% (GC) [3818, 23238-40-6]; Ethylene glycol monododecyl ether BioXtra, ≥99.0% (GC) [3819, 4536-30-5]; Ethylene glycol monohexadecyl ether BioXtra, ≥99.0% (TLC) [3820, 2136-71-2]; Ethylene glycol monohexyl ether BioXtra, ≥99.0% (GC) [3823, 112-25-4]; Ethylene glycol monooctyl ether ≥98.0% (GC) [3822, 10020-43-6]; Ethylene glycol monopentyl ether ≥90% (GC) [3825, 6196-58-3]; Ethylhexadecyldimethylammonium bromide ≥98% (non-aqueous titration) [C0636, 124-03-8]; Ethylhexadecyldimethylammonium bromide BioXtra [C5335, 124-03-8]; Genapol® C-100 [61028, 61791-13-7]; Genapol® X-080 [48750, 9043-30-5]; Girard's reagent T 99% [G900, 123-46-6]; Girard's reagent T crystalline [C1757, 123-46-6]; Glucopone 215 CS UP solution ˜65% in H2O, liquid (faintly turbid viscous) [49121, 170905-55-2]; Glucopone 600 CS UP solution ˜50% in H2O, paste [49122, 170905-55-2]; Glucopone 600 EC solution ˜50% in H2O, paste [49123, 170905-55-2]; Glycocholic acid hydrate synthetic, ≥97% (TLC) [G2878, 475-31-0]; Glycodeoxycholic acid monohydrate ≥97% (TLC) [G6132, 360-65-6]; Heptadecafluorooctanesulfonic acid lithium salt solution technical, ˜25% in H2O (T) [77281, 29457-72-5]; Heptaethylene glycol monododecyl ether BioXtra, ≥98.0% (TLC) [51554, 3055-97-8]; Heptaethylene glycol monohexadecyl ether ≥97.0% (TLC) [51555, 4486-31-1]; Heptyl-β-D-1-thioglucoside 300 mM solution [39489, 85618-20-8]; Hexadecyl(2-hydroxyethyl)dimethylammonium dihydrogen phosphate solution ˜30% in H2O [49581, 85563-48-0]; Hexadecylpyridinium bromide [C5881, 140-72-7]; Hexadecylpyridinium bromide ≥97.0% [52340, 140-72-7]; Hexadecylpyridinium chloride monohydrate 99.0-102.0% [C9002, 6004-24-6]; Hexadecylpyridinium chloride monohydrate BioXtra, ≥99.0% [C5460, 6004-24-6]; Hexadecylpyridinium chloride monohydrate Ph Eur [52350, 6004-24-6]; Hexadecyltrimethylammonium bromide ≥96.0% (AT) [52370, 57-09-0]; Hexadecyltrimethylammonium bromide ≥98%, powder [H5882, 57-09-0]; Hexadecyltrimethylammonium bromide analytical standard [36932, 57-09-0]; Hexadecyltrimethylammonium bromide BioUltra, ≥99.0% (AT) [52369, 57-09-0]; Hexadecyltrimethylammonium bromide BioUltra, for molecular biology, ≥99.0% (AT) [52365, 57-09-0]; Hexadecyltrimethylammonium bromide BioXtra, ≥99% [H9151, 57-09-0]; Hexadecyltrimethylammonium bromide for ion pair chromatography, ≥99.0% [52367, 57-09-0]; Hexadecyltrimethylammonium bromide for molecular biology, ≥99% [H6269, 57-09-0]; Hexadecyltrimethylammonium chloride purum, ≥98.0% (NT) [52366, 112-02-7]; Hexadecyltrimethylammonium chloride solution purum, ˜25% in H2O [52372, 112-02-7]; Hexadecyltrimethylammonium p-toluenesulfonate [C8147, 138-32-9]; Hexaethylene glycol monodecyl ether BioXtra, ≥99.0% (GC) [52043, 5168-89-8]; Hexaethylene glycol monododecyl ether BioXtra, ≥98.0% (TLC) [52044, 3055-96-7]; Hexaethylene glycol monododecyl ether semisolid [P8675, 3055-96-7]; Hexaethylene glycol monohexadecyl ether BioXtra, ≥99.0% (TLC) [52046, 5168-91-2]; Hexaethylene glycol monooctadecyl ether ≥99.0% (TLC) [52047, 2420-29-3]; Hexaethylene glycol monotetradecyl ether BioXtra, ≥99.0% (GC) [52048, 5157-04-0]; Hexyl J3-D-glucopyranoside ≥98.0% (TLC) [53180, 59080-45-4]; Hyamine® 1622 ≥96.0% (AT) [53752, 121-54-0]; Hyamine® 1622 BioUltra, ≥99.0% (AT) [53751, 121-54-0]; IGEPAL® CA-210 average Mn ˜294 [238562, 9036-19-5]; IGEPAL® CA-520 average Mn ˜427 [238570, 9036-19-5]; IGEPAL® CA-630 CMC 0.083 mM [56741, 9036-19-5]; IGEPAL® CA-630 for electrophoresis, suitable for 2-D electrophoresis [17771, 9036-19-5]; IGEPAL® CA-630 for molecular biology [18896, 9036-19-5]; IGEPAL® CA-630 viscous liquid [13021, 9036-19-5]; IGEPAL® CA-720 average Mn ˜735 [238589, 9036-19-5]; Imbentin AGS/35 [11510,]; Imbentin-AGS/35 25% in H2O [56742,]; Imbentin-N/63 in accordance for TLC [73334, 9016-45-9]; Isopropyl β-D-1-thiogalactopyranoside ≥99% (TLC) [I5502, 367-93-1]; Isopropyl β-D-1-thiogalactopyranoside ≥99% (TLC), ≤0.1% Dioxane [I6758, 367-93-1]; Isopropyl β-D-thiogalactopyranoside solution ReadyMade [11284, 367-93-1]; Lithium 3,5-diiodosalicylate [D3635, 653-14-5]; Lithium dodecyl sulfate ≥98.5% (GC) [L4632, 2044-56-6]; Lithium dodecyl sulfate BioReagent, for molecular biology, suitable for electrophoresis [L9781, 2044-56-6]; Lithium dodecyl sulfate BioReagent, suitable for electrophoresis, ˜99% (GC) [L2274, 2044-56-6]; Lithium dodecyl sulfate BioXtra, ≥98.5% (GC) [L5901, 2044-56-6]; Lugol solution [L6146,]; Lutensol® FSA 10 [17747, 31799-71-0]; Lutrol® OP 2000 [52639, 31394-71-5]; Luviquat™ FC 370-40% active ingredients in H2O [59061, 95144-24-4]; Luviquat™ FC 550-40% active ingredients in H2O [55332, 95144-24-4]; Luviquat™ HM 552-20% active ingredients in H2O [59059, 95144-24-4]; Luviquat™ HOLD ˜20% active ingredients in H2O [65691, 174761-16-1]; Luviquat™ Mono LS ˜30% (w/w) solids basis [73430, 68002-60-8]; Luviquat™ MS 370-40% active ingredients in H2O [53123, 150599-70-5]; L-α-Lysophosphatidylcholine from bovine brain ≥99%, Type V [L1381, 9008-30-4]; L-α-Lysophosphatidylcholine from egg yolk ≥99%, Type I, powder [L4129, 9008-30-4]; L-α-Lysophosphatidylcholine from Glycine max (soybean) ≥99%, lyophilized powder [L0906, 9008-30-4]; L-α-Lysophosphatidylcholine from soybean, ≥98.0% (10 mg phospholipid per ml CHC13, TLC) [62963,]; MEGA-8 500 mM solution [39490, 85316-98-9]; Methoxypolyethylene glycol 2,000 [M7143, 9004-74-4]; Methoxypolyethylene glycol 350 average mol wt 350 [M6768, 9004-74-4]; Methyl 6-O—(N-heptylcarbamoyl)-α-D-glucopyranoside ≥97.0% (TLC) [67328, 115457-83-5]; Methylbenzethonium chloride [M7379, 25155-18-4]; Miltefosine ≥98% (perchloric acid titration) [M5571, 58066-85-6]; Miltefosine hydrate ≥98% (HPLC) [M9198,]; Myristyltrimethylammonium bromide ≥98.0% (AT) [87210, 1119-97-7]; Myristyltrimethylammonium bromide ≥99% [T4762, 1119-97-7]; Myristyltrimethylammonium bromide puriss. p.a., for ion pair chromatography, ≥99.0% (AT) [87208, 1119-97-7]; N,N′,N′-Polyoxyethylene (10)-N-tallow-1,3-diaminopropane liquid [P4182, 61790-85-0]; N,N-Bis[3-(D-gluconamido)propyl]deoxycholamide ≥90% (HPLC) [14840, 86303-23-3]; N,N-Dimethyldecylamine N-oxide ≥99.0% (NT) [40103, 2605-79-0]; N,N-Dimethyldodecylamine N-oxide ≥99% (titration) [D9775, 1643-20-5]; N,N-Dimethyldodecylamine N-oxide BioXtra, ≥99.0% (NT) [40234, 1643-20-5]; N,N-Dimethyldodecylamine N-oxide solution BioUltra, ˜0.1 M in H2O [40231, 1643-20-5]; N,N-Dimethyldodecylamine N-oxide solution purum, ˜30% in H2O [40236, 1643-20-5]; N,N-Dimethylhexylamine-N-oxide ≥99.0% (NT) [40663, 34418-88-7]; N,N-Dimethyloctadecylamine N-oxide solution paste, ˜25% in H2O [40965, 2571-88-2]; N,N-Dimethyltridecylamine N-oxide ≥97.0% (NT) [41713, 5960-96-3]; N-Decanoyl-N-methylglucamine ≥98% (GC) [D6277, 85261-20-7]; n-Decyl α-D-glucopyranoside ˜98% (GC) [D5419, 29781-81-5]; n-Dodecyl β-D-glucopyranoside ≥98% (GC) [D8035, 59122-55-3]; n-Dodecyl β-D-maltoside ≥98% (GC) [D4641, 69227-93-6]; n-Dodecyl β-D-maltoside BioXtra, ≥98% (GC) [D5172, 69227-93-6]; N-Dodecyl-N,N-dimethyl-3-ammonio-1-propanesulfonate [D0431, 14933-08-5]; N-Dodecyl-N,N-dimethyl-3-ammonio-1-propanesulfonate ≥97.0% (dried material, CHN) [40232, 14933-08-5]; n-Heptyl β-D-glucopyranoside ˜98% (GC) [H1389, 78617-12-6]; n-Heptyl β-D-glucopyranoside ≥98.0% (TLC) [51980, 78617-12-6]; n-Heptyl 3-D-thioglucopyranoside ≥99% (GC) [H3264, 85618-20-8]; n-Hexadecyl 3-D-maltoside ≥99.0% (TLC) [52318, 98064-96-1]; Niaproof® 4-27% in H2O [4, 139-88-8]; Niaproof® 4-27% in H2O [N1404, 139-88-8]; Niaproof® 4 BioXtra, ˜27% in H2O [72216, 139-88-8]; N-Lauroyl-L-alanine ≥99.0% (TLC) [61726, 52558-74-4]; N-Lauroylsarcosine purum p.a., ≥98.0% (GC) [61739, 97-78-9]; N-Lauroylsarcosine sodium salt ≥94% [L5125, 137-16-6]; N-Lauroylsarcosine sodium salt BioReagent, for molecular biology, ≥94% [L9150, 137-16-6]; N-Lauroylsarcosine sodium salt BioUltra, for molecular biology, ≥99.0% (HPLC) [61743, 137-16-6]; N-Lauroylsarcosine sodium salt BioXtra, ≥97% (TLC) [L5777, 137-16-6]; N-Lauroylsarcosine sodium salt purum, ≥97.0% (HPLC) [61745, 137-16-6]; N-Lauroylsarcosine sodium salt solution 20%, for molecular biology [L7414, 137-16-6]; N-Lauroylsarcosine sodium salt solution 30% aqueous solution, ≥97.0% (HPLC) [61747, 137-16-6]; N-Lauroylsarcosine, neat ≥95% [L5000, 97-78-9]; N-Nonanoyl-N-methylglucamine ≥98% [N1138, 85261-19-4]; N-Nonanoyl-N-methylglucamine ≥98.0% (TLC) [74315, 85261-19-4]; N-Octanoyl-N-methylglucamine ˜98% [03129, 85316-98-9]; N-Octanoyl-N-methylglucamine 98% [343897, 85316-98-9]; N-Octanoyl-N-methylglucamine BioXtra, ≥98.0% (TLC) [74881, 85316-98-9]; N-Octanoyl-β-D-glucosylamine ≥98.0% (TLC) [74878, 134403-86-4]; n-Octyl 3-D-maltoside ≥99.0% (HPLC) [19181, 82494-08-4]; Nonaethylene glycol monododecyl ether [P9641, 3055-99-0]; Nonaethyleneglycol monododecyl ether 1 mM solution [40913, 3055-99-0]; Nonidet™ P 40 Substitute BioXtra, mixture of 15 homologues [74385, 9016-45-9]; Nonidet™ P 40 Substitute solution BioXtra, ampule, ˜10% in H2O [74388, 9016-45-9]; Nonyl 3-D-glucopyranoside ˜98% (GC) [N7507, 69984-73-2]; Nonyl 3-D-glucopyranoside ≥98.0% (TLC) [74420, 69984-73-2]; Nonyl β-D-maltoside ≥99.0% (TLC) [59965, 106402-05-5]; Nonylphenyl-polyethyleneglycol acetate for histology [74432, 54612-40-7]; Nonyl-β-D-1-thiomaltoside ≥98.0% (TLC) [74436, 148565-55-3]; n-Undecyl 3-D-glucopyranoside ˜97% (GC) [U5254, 70005-86-6]; Na-Lauroyl-L-lysine ≥99.0% (TLC) [61734, 59409-41-5]; O-(Decylphosphoryl)choline solution [55461, 70504-28-8]; 0-(Octylphosphoryl)choline solution 1 M in H2O [38544, 53255-89-3]; Octaethylene glycol monodecyl ether BioXtra, ≥98.0% (TLC) [74716, 24233-81-6]; Octaethylene glycol monododecyl ether ≥98% (GC) [P8925, 3055-98-9]; Octaethylene glycol monododecyl ether BioXtra, ≥98.0% (GC) [74680, 3055-98-9]; Octaethylene glycol monohexadecyl ether BioXtra, ≥98.0% (GC) [74717, 5698-39-5]; Octaethylene glycol monohexadecyl ether powder [P0676, 5698-39-5]; Octaethylene glycol monooctadecyl ether BioXtra, ≥98.0% (TLC) [74718, 13149-87-6]; Octaethylene glycol monotetradecyl ether [P9800, 27847-86-5]; Octaethylene glycol monotetradecyl ether ≥99.0% (TLC) [74719, 27847-86-5]; Octaethyleneglycol monododecyl ether 1 mM solution [55377, 3055-98-9]; Octyl D-glucopyranoside mixture of anomers, α-anomer/β-anomer ˜7:3, ˜80% (TLC) [75081, 54549-23-4]; Octyl 3-D-1-thioglucopyranoside ≥98% (GC) [06004, 85618-21-9]; Octyl 3-D-1-thioglucopyranoside ≥99.0% (TLC) [75082, 85618-21-9]; Octyl 3-D-galactopyranoside ≥98% (GC) [00261, 40427-75-6]; Octyl 3-D-glucopyranoside ≥98% (GC) [08001, 29836-26-8]; Octyl 3-D-glucopyranoside ≥99% (GC) [39473, 29836-26-8]; Octyl 3-D-glucopyranoside BioXtra, ≥98% (GC) [09882, 29836-26-8]; Octyl 3-D-glucopyranoside solution BioReagent, suitable for electrophoresis, 50% (w/v) in H2O [03757, 29836-26-8]; Octyl-α/β-glucoside 10 mM solution [30887, 54549-23-4]; Octyl-β-D-glucopyranoside 100 mM solution [18842, 29836-26-8]; Oxyphenonium bromide [05501, 50-10-2]; Pentaethylene glycol monodecyl ether BioXtra, ≥97.0% (GC) [76436, 23244-49-7]; Pentaethylene glycol monododecyl ether ≥98% (GC) [P8550, 3055-95-6]; Pentaethylene glycol monododecyl ether BioXtra, ≥98.0% (GC) [76437, 3055-95-6]; Pentaethylene glycol monohexadecyl ether BioXtra, ≥99.0% (TLC) [76439, 4478-97-1]; Pentaethylene glycol monohexyl ether ≥90% (GC) [P4529, 86674-95-5]; Pentaethylene glycol monooctyl ether [P4654, 19327-40-3]; Pentaethylene glycol monotetradecyl ether ≥99.0% (TLC) [76442, 92669-01-7]; Pluronic® F-68 solution 10%, BioReagent, solution, sterile-filtered, suitable for insect cell culture [P5556, 106392-12-5]; Pluronic® F-127 powder, BioReagent, suitable for cell culture [P2443, 2594628]; Pluronic® F-68 plant cell culture tested, BioReagent [P7061, 2594628]; Pluronic® F-68 solid, cell culture tested, insect cell culture tested [P1300, 2594628]; Poloxamer 407 purified, non-ionic [16758, 2594628]; Poly(ethylene glycol) average mol wt 10,000 [P6667, 25322-68-3]; Poly(ethylene glycol) diglycidyl ether ˜75% [P2672, 72207-80-8]; Poly(ethylene glycol) for molecular biology, average mol wt 8,000 [P5413, 25322-68-3]; Poly(ethylene glycol) methyl ether 2,000 [81321, 9004-74-4]; Poly(ethylene glycol) methyl ether 350 [81318, 9004-74-4]; Poly(ethylene glycol) methyl ether 500 [81316, 9004-74-4]; Poly(ethylene glycol) methyl ether BioUltra, 500 [71578, 9004-74-4]; Poly(maleic anhydride-alt-1-decene), 3-(dimethylamino)-1-propylamine derivative BioReagent, for molecular biology [67228, 869856-84-8]; Poly(maleic anhydride-alt-1-dodecene), 3-(Dimethylamino)-1-propylamine derivative BioReagent, for molecular biology [76025, 869856-88-2]; Poly(maleic anhydride-alt-1-octadecene), 3-(Dimethylamino)-1-propylamine derivative [74927, 869857-16-9]; Poly(maleic anhydride-alt-1-octene), 3-(dimethylamino)-1-propylamine derivative [90779, 869856-83-7]; Poly(maleic anhydride-alt-1-tetradecene), 3-(dimethylamino)-1-propylamine derivative BioReagent, for molecular biology [90771, 869857-14-7]; Polyoxyethylene (10) tridecyl ether [P2393, 24938-91-8]; Polyoxyethylene (100) stearate [P3690, 9004-99-3]; Polyoxyethylene (20) oleyl ether [P5641, 9004-98-2]; Polyoxyethylene (20) sorbitan monolaurate solution 10% in H2O [P8942, 9005-64-5]; Polyoxyethylene (20) sorbitan monolaurate solution ampule, ˜10% in H2O [93774, 9005-64-5]; Polyoxyethylene (40) stearate [P3440, 9004-99-3]; Polyoxyethylene (7) stearyl ether ˜95% [P1301, 66146-84-7]; Polyoxyethylene bis(imidazolyl carbonyl) [P5532,]; Polyoxyethylene (25) propylene glycol stearate [P2440, 37231-60-0]; Polysorbat 60 Ph Eur [95754, 9005-67-8]; Polysorbate 20 Ph Eur [44112, 9005-64-5]; Polysorbate 80 Ph Eur [59924, 9005-65-6]; Potassium nonafluoro-1-butanesulfonate purum, ≥97.0% (T) [60418, 29420-49-3]; Pril® neutral detergent [40643,]; Saponin [84510, 8047-15-2]; Saponin for molecular biology [47036, 8047-15-2]; Saponin from quillaja bark Sapogenin content ≥10% [S7900, 8047-15-2]; Saponin from quillaja bark Sapogenin content 20-35% [S4521, 8047-15-2]; Sodium 1-butanesulfonate [B2013, 2386-54-1]; Sodium 1-butanesulfonate ≥95.0% (T) [19028, 2386-54-1]; Sodium 1-butanesulfonate for ion pair chromatography, ≥99.0% (T) [19022, 2386-54-1]; Sodium 1-butanesulfonate puriss., ≥99.0% (T) [19024, 2386-54-1]; Sodium 1-decanesulfonate ˜98% [D3412, 13419-61-9]; Sodium 1-decanesulfonate ≥99.0% (T) [30633, 13419-61-9]; Sodium 1-decanesulfonate BioXtra, ˜98% [D0181, 13419-61-9]; Sodium 1-decanesulfonate for ion pair chromatography, ≥99.0% [30631, 13419-61-9]; Sodium 1-dodecanesulfonate [D5266, 2386-53-0]; Sodium 1-heptanesulfonate [H2766, 22767-50-6]; Sodium 1-heptanesulfonate BioXtra [H8901, 22767-50-6]; Sodium 1-heptanesulfonate monohydrate puriss. p.a., for ion pair chromatography, ≥99.0% (T) [51832, 207300-90-1]; Sodium 1-heptanesulfonate monohydrate puriss., ≥99.0% (T) [51833, 207300-90-1]; Sodium 1-hexanesulfonate monohydrate puriss. p.a., for ion pair chromatography, ≥99.0% (T) [52862, 207300-91-2]; Sodium 1-nonanesulfonate ˜98% [N0893, 35192-74-6]; Sodium 1-nonanesulfonate ≥97.0% (T) [74318, 35192-74-6]; Sodium 1-nonanesulfonate puriss. p.a., for ion pair chromatography, ≥99.0% (T) [74316, 35192-74-6]; Sodium 1-octanesulfonate monohydrate puriss. p.a., for ion pair chromatography, ≥99.0% (T) [74882, 207596-29-0]; Sodium 1-octanesulfonate monohydrate puriss., ≥99.0% (T) [74884, 207596-29-0]; Sodium 1-pentanesulfonate monohydrate puriss. p.a., for ion pair chromatography, ≥99.0% (T) [76952, 207605-40-1]; Sodium 1-propanesulfonate monohydrate [P4009, 304672-01-3]; Sodium 1-propanesulfonate monohydrate puriss. p.a., for ion pair chromatography, ≥99.0% (T) [81806, 304672-01-3]; Sodium 1-propanesulfonate monohydrate purum, ≥97.0% (T) [81805, 304672-01-3]; Sodium 1-undecanesulfonate puriss. p.a., ≥99.0% (T) [94133, 5838-34-6]; Sodium 2,3-dimercaptopropanesulfonate monohydrate ˜95% [D8016, 207233-91-8]; Sodium 2-bromoethanesulfonate purum, ≥95.0% (T) [16151, 4263-52-9]; Sodium 2-ethylhexyl sulfate purum, ˜50% in H2O [71220, 126-92-1]; Sodium chenodeoxycholate ≥97% [C8261, 2646-38-0]; Sodium cholate hydrate ≥97.0% (dried material, NT) [27029, 206986-87-0]; Sodium cholate hydrate BioXtra, ≥99% [C6445, 206986-87-0]; Sodium cholate hydrate from ox or sheep bile, ≥99% [C1254, 206986-87-0]; Sodium cholate hydrate suitable for cell culture, BioReagent [C9282, 206986-87-0]; Sodium choleate [S9875,]; Sodium cholesteryl sulfate [C9523, 2864-50-8]; Sodium cholesteryl sulfate purum, ≥98.0% (TLC) [26955, 2864-50-8]; Sodium dehydrocholate ˜95% [D3875, 145-41-5]; Sodium dehydrocholate ≥98.0% (calc. based on dry substance, NT) [30850, 145-41-5]; Sodium deoxycholate ≥97% (titration) [D6750, 302-95-4]; Sodium deoxycholate BioXtra, ≥98.0% (dry matter, NT) [30970, 302-95-4]; Sodium deoxycholate monohydrate BioUltra, ≥99.0% (NT) [30968, 145224-92-6]; Sodium deoxycholate monohydrate BioXtra, ≥99.0% (titration) [D5670, 145224-92-6]; Sodium dodecyl sulfate ≥90% [62862, 151-21-3]; Sodium dodecyl sulfate ≥96.0% (GC) [71728, 151-21-3]; Sodium dodecyl sulfate ≥98.0% (GC) [55422, 151-21-3]; Sodium dodecyl sulfate ≥98.0% (GC) [71729, 151-21-3]; Sodium dodecyl sulfate ≥98.0% (GC), dust-free pellets [75746, 151-21-3]; Sodium dodecyl sulfate 92.5-100.5% based on total alkyl sulfate content basis [L5750, 151-21-3]; Sodium dodecyl sulfate ACS reagent, ≥99.0% [436143, 151-21-3]; Sodium dodecyl sulfate BioUltra, ≥99.0% (GC) [71727, 151-21-3]; Sodium dodecyl sulfate BioUltra, for molecular biology, ≥99.0% (GC) [71725, 151-21-3]; Sodium dodecyl sulfate BioXtra, ≥99.0% (GC) [L6026, 151-21-3]; Sodium dodecyl sulfate for electrophoresis, ≥98.5% (GC) [L3771, 151-21-3]; Sodium dodecyl sulfate for ion pair chromatography, ≥99.0% [71726, 151-21-3]; Sodium dodecyl sulfate for molecular biology, ≥98.5% (GC) [L4390, 151-21-3]; Sodium dodecyl sulfate ReagentPlus®, ≥98.5% (GC) [L4509, 151-21-3]; Sodium dodecyl sulfate solution BioUltra, for molecular biology, 10% in H2O [71736, 151-21-3]; Sodium dodecyl sulfate solution BioUltra, for molecular biology, 20% in H2O [5030, 151-21-3]; Sodium dodecyl sulfate solution for molecular biology, 10% in 18 megohm water [L4522, 151-21-3]; Sodium dodecyl sulfate technical, ≥90.0% (GC) [71730, 151-21-3]; Sodium dodecyl sulfate USP/NF, mixture of sodium alkyl sulfates consisting mainly of sodium dodecyl sulfate [71717, 151-21-3]; Sodium glycochenodeoxycholate ≥97% (TLC) [G0759, 16564-43-5]; Sodium glycochenodeoxycholate ≥97.0% (TLC) [50534, 16564-43-5]; Sodium glycocholate hydrate ≥97% (TLC) [G7132, 338950-81-5]; Sodium glycodeoxycholate BioXtra, ≥97% (TLC) [G9910, 16409-34-0]; Sodium hexanesulfonate ≥98% (elemental analysis) [H5269, 2832-45-3]; Sodium hexanesulfonate BioXtra [H9026, 2832-45-3]; Sodium octanoate ≥99% (capillary GC) [C5038, 30834]; Sodium octanoate puriss., ≥98.5% (NT) [71339, 30834]; Sodium octyl sulfate ˜95% [04003, 142-31-4]; Sodium octyl sulfate puriss. p.a., for ion pair chromatography, ≥99.0% (T) [75073, 142-31-4]; Sodium pentanesulfonate ≥95% (elemental analysis) [P0299, 22767-49-3]; Sodium pentanesulfonate BioXtra [P8199, 22767-49-3]; Sodium taurochenodeoxycholate [T6260, 6009-98-9]; Sodium taurocholate hydrate ≥97.0% (TLC) [86339, 345909-26-4]; Sodium taurodeoxycholate hydrate ≥95% (TLC) [T0875, 207737-97-1]; Sodium taurodeoxycholate hydrate BioXtra, ≥97% (TLC) [T0557, 207737-97-1]; Sodium taurohyodeoxycholate hydrate ≥98% [T0682, 38411-85-7]; Sodium taurolithocholate [T7515, 6042-32-6]; Sodium tauroursodeoxycholate ˜90% [T0266, 14605-22-2]; SODOSIL® RA 32 clear rinse [16182,]; SODOSIL® RAM 05 acid cleaner with formic acid [16186,]; SODOSIL® RM 003 phosphate-free cleaner [16177,]; SODOSIL® RM 01 alkaline cleaner [16166,]; SODOSIL® RM 02 neutral cleaner [16167,]; Span® 20 [S6635, 1338-39-2]; Span® 60 [S7010, 1338-41-6]; Span® 65 [85547, 26658-19-5]; Span® 65 [S2028, 26658-19-5]; Span® 80 [S6760, 1338-43-8]; Span® 80 for GC [9569, 1338-43-8]; Span® 80 viscosity 1200-2000 mPa·s (20° C.) [85548, 1338-43-8]; Span® 85 [S7135, 26266-58-0]; Steareth-100 pellets, white [50216, 9005-00-9]; Sucrose monodecanoate [S1266, 31835-06-0]; Sucrose monolaurate BioXtra, ≥97.0% (TLC) [84110, 25339-99-5]; Surfactin from Bacillus subtilis, ≥98% (HPLC and TLC) [S3523, 24730-31-2]; Synperonic® F 108 surfactant, non-ionic [7579, 2594628]; Synperonic® L 122 surfactant [86218, 2594628]; Synperonic® NP 10 [86208, 9016-45-9]; Synperonic® NP 30 [86209, 9016-45-9]; Synperonic® P 85 surfactant [86213, 2594628]; Synperonic® PE P105 surfactant [86216, 2594628]; Synperonic® PE/F68 stationary phase for GC, block copolymer of polyethylene and polypropylene glycol [81112, 2594628]; Synperonic® PE/L61 liquid phase for GC, block copolymer of polyethylene and polypropylene glycol [81113, 2594628]; Synperonic® PE/L64 block copolymer of polyethylene and polypropylene glycol, liquid phase for GC [81114, 2594628]; Taurocholic acid sodium salt hydrate ≥95% (TLC) [T4009, 345909-26-4]; Taurocholic acid sodium salt hydrate BioXtra, ≥95% (TLC) [T9034, 345909-26-4]; Taurolithocholic acid 3-sulfate disodium salt [T0512, 64936-83-0]; Teepol® 610 S [86350,]; Tergitol® MIN FOAM 1× [T1135, 68551-14-4]; Tergitol® NP-9 [17881, 9016-45-9]; Tergitol® solution Type NP-40, 70% in H2O [NP40S,]; Tergitol® TMN 10 90% active ingredients basis [86454, 60828-78-6]; Tergitol® TMN 6 90% active ingredients basis [86453, 60828-78-6]; Tergitol® Type 15-S-12 [15S12, 68131-40-8]; Tergitol® Type 15-S-30 [15S30, 68131-40-8]; Tergitol® Type 15-S-5 [15S5, 68131-40-8]; Tergitol® Type 15-S-7 [15S7, 68131-40-8]; Tergitol® Type 15-S-9 [15S9, 68131-40-8]; Tergitol® Type NP-10 [NP10, 127087-87-0]; Tergitol® Type NP-35 [NP35, 127087-87-0]; Tergitol® Type NP-7 [NP7, 127087-87-0]; Tergitol® Type NP-9 [NP9, 127087-87-0]; Tetradecyl-β-D-maltoside ≥99.0% (TLC) [15826, 18449-82-6]; Tetraethylene glycol monodecyl ether ˜97% (GC) [P7425, 5703-94-6]; Tetraethylene glycol monodecyl ether ≥97.0% (GC) [86693, 5703-94-6]; Tetraethylene glycol monododecyl ether BioXtra, ≥98.0% (GC) [86694, 5274-68-0]; Tetraethylene glycol monohexadecyl ether [P0176, 5274-63-5]; Tetraethylene glycol monohexadecyl ether ≥99.0% (TLC) [86695, 5274-63-5]; Tetraethylene glycol monooctadecyl ether BioXtra, ≥99.0% (GC) [86696, 59970-10-4]; Tetraethylene glycol monooctyl ether BioXtra, ≥98.0% (GC) [86698, 19327-39-0]; Tetraethylene glycol monooctyl ether liquid, ≥98% (GC) [T3394, 19327-39-0]; Tetraethylene glycol monotetradecyl ether [P9300, 39034-24-7]; Tetraethylene glycol monotetradecyl ether ≥99.0% (TLC) [86697, 39034-24-7]; Tetraglycol BioXtra [T3396, 31692-85-0]; Tetraheptylammonium bromide puriss. p.a., for ion pair chromatography, ≥99.0% (AT) [87296, 4368-51-8]; Tetrakis(decyl)ammonium bromide puriss. p.a., for ion pair chromatography, ≥99.0% (AT) [87578, 14937-42-9]; Tetramethylammonium hydroxide pentahydrate ≥97% [T7505, 10424-65-4]; Thesit® for membrane research [88315, 9002-92-0]; Thesit® solution for membrane research, 10% in H2O [88317, 9002-92-0]; Thonzonium bromide [T7783, 553-08-2]; Tricaprylylmethylammonium chloride mixture of C8-C10 C8 is dominant [91042, 63393-96-4]; Tridecyl 3-D-maltoside ≥99.0% (TLC) [16321, 93911-12-7]; Tridodecylmethylammonium chloride purum, ≥97.0% (AT) [91662, 7173-54-8]; Tridodecylmethylammonium chloride Selectophore® [91661, 7173-54-8]; Triethylene glycol monodecyl ether BioXtra, ≥97.0% (GC) [90446, 4669-23-2]; Triethylene glycol monododecyl ether ≥98% (GC) [P8425, 3055-94-5]; Triethylene glycol monoheptyl ether ≥98.0% (GC) [90444, 55489-59-3]; Triethylene glycol monomethyl ether purum, ≥97.0% (GC) [90450, 112-35-6]; Triethylene glycol monooctyl ether BioXtra, ≥98.0% (GC) [90455, 19327-38-9]; Trimethyloctadecylammonium bromide purum, ≥97.0% (AT) [74765, 1120-02-1]; Triton® N-57 [N57,]; Triton® N-57 [N57,]; Triton® N-60 [N60,]; Triton® N-60 [N60,]; Triton® QS-15 [QS15, 11105-10-5]; Triton® X-100 [21123, 9002-93-1]; Triton® X-100 BioXtra [T9284, 9002-93-1]; Triton® X-100 BioXtra [93418, 9002-93-1]; Triton® X-100 BioXtra, for molecular biology [93426, 9002-93-1]; Triton® X-100 for electrophoresis [T8532, 9002-93-1]; Triton® X-100 for electrophoresis [T8532, 9002-93-1]; Triton® X-100 for molecular biology [T8787, 9002-93-1]; Triton® X-100 for molecular biology [T8787, 9002-93-1]; Triton® X-100 laboratory grade [X100, 9002-93-1]; Triton® X-100 non-ionic detergent [93420, 9002-93-1]; Triton® X-100 peroxide- and carbonyl-free [X100PC, 9002-93-1]; Triton® X-100 reduced [X100RS, 92046-34-9]; Triton® X-100 solution ampule, ˜10% in H2O [93427, 9002-93-1]; Triton® X-100 solution ampule, ˜10% in H2O [93427, 9002-93-1]; Triton® X-100 solution BioUltra, for molecular biology, ˜10% in H2O [93443, 9002-93-1]; Triton® X-100 solution BioUltra, for molecular biology, ˜10% in H2O [93443, 9002-93-1]; Triton® X-100, reduced [28468, 92046-34-9]; Triton® X-100, reduced form non-ionic detergent [93424, 92046-34-9]; Triton® X-102 [X102, 9002-93-1]; Triton® X-102 [X102, 9002-93-1]; Triton® X-114 [93422, 9036-19-5]; Triton® X-114 [93422, 9036-19-5]; Triton® X-114 laboratory grade [X114, 9036-19-5]; Triton® X-114 laboratory grade [X114, 9036-19-5]; Triton® X-114 solution ampule, ˜10% in H2O [93428, 9036-19-5]; Triton® X-114 solution ampule, ˜10% in H2O [93428, 9036-19-5]; Triton® X-15 [X15, 9036-19-5]; Triton® X-15 [X15, 9036-19-5]; Triton® X-151 solution 85% in H2O [X151, 59979-06-5]; Triton® X-165 solution 70% in H2O [X165, 9036-19-5]; Triton® X-165 solution 70% in H2O [X165, 9036-19-5]; Triton® X-200 solution 28% in H2O (dispersion) [X200, 9010-41-7]; Triton® X-207 [X207, 9036-19-5]; Triton® X-207 [X207, 9036-19-5]; Triton® X-305 solution ˜70% in H2O [93445, 9002-93-1]; Triton® X-305 solution 70% in H2O [X305, 9002-93-1]; Triton® X-305 solution 70% in H2O [X305, 9002-93-1]; Triton® X-405 solution 70% in H2O [X405, 9036-19-5]; Triton® X-405 solution 70% in H2O [X405, 9036-19-5]; Triton® X-45 [X45, 9002-93-1]; Triton® X-705 solution 70% in H2O [X70570, 9036-19-5]; Triton® X-705 solution 70% in H2O [X70570, 9036-19-5]; Triton® XQS-20 solution 70% in H2O [XQS20, 65256-19-1]; Triton® X-100 [234729, 9002-93-1]; Turkey red oil sodium salt [T5766, 8002-33-3]; Turkey red oil sodium salt 100% [3867, 8002-33-3]; Turkey red oil sodium salt for microscopy [16316, 8002-33-3]; TWEEN® 20 [63158, 9005-64-5]; TWEEN® 20 [T2700, 9005-64-5]; TWEEN® 20 average Mn ˜1,228 [274348, 9005-64-5]; TWEEN® 20 BioXtra, viscous liquid [P7949, 9005-64-5]; TWEEN® 20 for electrophoresis, suitable for solubilizing agent of membrane proteins and as a blocking reagent in immunoblotting [P5927, 9005-64-5]; TWEEN® 20 for molecular biology, viscous liquid [P9416, 9005-64-5]; TWEEN® 20 Low-peroxide; Low-carbonyls [P6585, 9005-64-5]; TWEEN® 20 Low-peroxide; Low-carbonyls [P8341, 9005-64-5]; TWEEN® 20 viscosity 250-450 mPa·s (25° C.) [93773, 9005-64-5]; TWEEN® 20 viscous liquid [P1379, 9005-64-5]; TWEEN® 20 viscous liquid, cell culture tested [P2287, 9005-64-5]; TWEEN® 21 [P2565, 9005-64-5]; TWEEN® 40 viscous liquid [P1504, 9005-66-7]; TWEEN® 60 [P1629, 9005-67-8]; Tween® 65 [93778, 9005-71-4]; TWEEN® 65 [P3190, 9005-71-4]; TWEEN® 80 [63161, 9005-65-6]; TWEEN® 80 BioXtra, viscous liquid [P8074, 9005-65-6]; TWEEN® 80 cell culture tested, viscous liquid [P4780, 9005-65-6]; TWEEN® 80 for molecular biology, syrup [P5188, 9005-65-6]; TWEEN® 80 from non-animal source [P6224, 9005-65-6]; TWEEN® 80 insect cell culture tested, viscous liquid [P4675, 9005-65-6]; TWEEN® 80 solution 10%, low peroxide [P0343, 9005-65-6]; TWEEN® 80 solution 10%, low peroxide [P8192, 9005-65-6]; TWEEN® 80 solution 10%, low peroxide [P0343, 9005-65-6]; TWEEN® 80 solution 10%, low peroxide [P8192, 9005-65-6]; TWEEN® 80 viscosity 375-480 mPa·s (25° C.) [93781, 9005-65-6]; TWEEN® 80 viscous liquid [P1754, 9005-65-6]; TWEEN® 80 viscous liquid, Low-peroxide [P6349, 9005-65-6]; TWEEN® 80 viscous liquid, Preservative Free, Low-peroxide; Low-carbonyls [P6474, 9005-65-6]; TWEEN® 85 [P4634, 9005-70-3]; Tyloxapol [T8761, 25301-02-4]; Tyloxapol BioXtra [T0307, 25301-02-4]; Undecyl β-D-maltoside ≥99.0% (TLC) [94206, 253678-67-0]; Ursodeoxycholic acid ≥99% [U5127, 128-13-2]; Zonyl® FS-300 nonionic fluorosurfactant [9996, 197664-69-0]; Zonyl® FSA anionic lithium carboxylate fluorosurfactant [9904, 57534-43-7]; Zonyl® FSE anionic phosphate fluorosurfactant [9989, 83653-37-6]; Zonyl® FSJ anionic phosphate surfactant; unique blend of fluorosurfactant (FSP, 09988) and a hydrocarbon surfactant [9991, 82784-95-0]; Zonyl® FSK amphoteric quaternary ammonium-acetate fluorosurfactant [9903, 80449-64-5]; Zonyl® FSN nonionic fluorosurfactant [9995, 65545-80-4]; Zonyl® FSO nonionic ethoxylated fluorosurfactant [9994, 65545-80-4]; Zonyl® FSP anionic phosphate fluorosurfactant [9988, 67479-86-1]; Zonyl® TBS NH4+-form, anionic sulfonate fluorosurfactant [9902, 111019-20-6]; Zonyl® UR anionic phosphate fluorosurfactant (100% solids) [9992, 99637-22-6].

Typical anionic detergents are alkylbenzenesulfonates. The alkylbenzene portion of these anions is lipophilic and the sulfonate is hydrophilic. Cationic detergents are similar to the anionic ones, with a hydrophobic component, but instead of the anionic sulfonate group, the cationic surfactants have quaternary ammonium as the polar end. The ammonium center is positively charged.

Ethoxylate detergents are compounds that have long hydrocarbon chains, but terminate with (OCH2CH2)nOH group. These groups are not charged, but they are highly hydrophilic owing to the presence of many oxygen centres. Non-ionic (or zwitterionic) detergents are characterized by their (net) uncharged, hydrophilic headgroups. They are based on polyoxyethylene glycol (i.e. Tween, Triton and Brij series), CHAPS, glycosides (i.e. octyl-thioglucoside, maltosides), bile acids such as DOC, lipids (HEGAs), or phosphine oxides.

### Salts

In preferred embodiments, the medium comprises a salt. Salts suitable for use according to the present invention may comprise one or more of the following cations: Hydrogen, Sodium, Lithium, Potassium, Rubidium, Cesium Silver, Beryllium, Magnesium, Calcium, Strontium, Barium, Radium, Aluminum, Bismuth, Hydrogen, Copper (I), Gold (I), Chromium (II), Copper (II), Cobalt (II), Iron (II), Lead (II), Manganese (II), Mercury(II), Tin (II), Chromium (III), Cobalt (III), Gold (III), Iron (III), Manganese (III), Lead (IV), Ammonium.

Salts suitable for use according to the present invention may comprise one or more of the following anions: hydride, oxide, nitride, carbide, fluoride, sulfide, phosphide, silicide, chloride, selenide, arsenide, bromide, telluride, iodide, cyanide, hydroxide, hypochlorite, sulfite, hypobromite, hypoiodite, chlorite, bromite, iodite, hydrogen sulfite (bisulfite), nitrite, chlorate, carbonate, bromate, chromate, iodate, dichromate, perchlorate, monohydrogen phosphate, perbromate, oxalate, periodate, sulfate, hydrogen carbonate, thiosulfate, hydrogen sulfate, dihydrogen phosphate, acetate, nitrate, permanganate, thiocyanate. In various embodiments a suitable salt may comprise one or more anions selected from; tetrafluoroborates, hexafluorophosphates, nitrates, carboxylates, benzoates, acetates, sulfates, tetraarylborates, arylsulfonates, alkylsulfonates, and halides. The cation portion of the salt can be at least one guanidinium salt or onium salt, including ammonium, phosphonium, or sulfonium salts that are substituted with organic residues. Illustrative examples of guanidinium salts include, but are not limited to, hexasubstituted guanidinium halides, such as hexaalkyl guanidinium halides, hexaaryl guanidinium halides, and hexasubstituted guanidinium halides containing mixtures of alkyl and aryl substituents each substituent group independently having a carbon number of 1 to 22; for example hexaalkylguanidinium chlorides or bromides. In one embodiment of the invention hexaethylguanidinium bromide is preferred. Illustrative examples of onium salts include, but are not limited to, tetraalkylammonium or tetraalkylphosphonium halides, sulfates, nitrates, p-tolylsulfonates, tetrafluoroborates, tetraarylborates, or hexafluorophosphates. In preferred embodiments the salts are halides such as the chlorides and bromides, particularly the bromides. Organic residues on the onium salts are typically include C.sub.6-10 aryl, C.sub.7-12 aralkyl, or C.sub.1-20 alkyl, or combinations thereof. Preferred onium salts are tetraalkylammonium halides containing primary and/or secondary alkyl groups containing about 1-8 carbon atoms. In one embodiment onium salts comprise tetraethylammonium, tetramethylammonium, tetrabutylammonium, or methyltributylammonium cations. Tetraethylammonium bromide is particularly preferred. In various embodiments a suitable salt may comprise one or more anions selected from; alkali metal cations. Accordingly, a non-exclusive listing of preferred alkali metal salts includes those with anions listed hereinabove, such as lithium bromide, sodium chloride, sodium bromide, potassium bromide, and cesium bromide. Examples of titanium sources include in organic titanium salts such as titanium (IV) bromide, titanium (IV) chloride; titanium alkoxides and aryloxides such as titanium (IV) methoxide, titanium (IV) ethoxide, titanium (IV) isopropoxide, titanium (IV) 2-ethylhexoxide, titanium (IV) butoxide, titanium (IV) 2-ethyl-1,3-hexanediolate, titanium (IV) (triethanolaminato)isopropoxide and titanium (IV) phenoxide; and titanium salts of .beta.-diketones or .beta.-ketoesters such as titanium (IV) diisopropoxide bis(acetylacetonate), titanium (IV) bis(ethyl acetoacetato)diisopropoxide, titanium (IV) oxide bis(2,4-pentanedionate) (or titanium (IV) oxide acetylacetonate). Mixtures of titanium compounds may also be employed. Examples of manganese sources (sometimes referred to hereinafter as manganese compound) include manganese halides, manganese chloride, manganese bromide, manganese nitrate, manganese carboxylates such as manganese (II) acetate, and manganese salts of .beta.-diketones such as manganese (III) 2,4-pentanedionate and manganese (II) 2,4-pentanedionate (manganese (II) acetylacetonate). Mixtures of manganese compounds may also be employed.

### Step C. Providing a Suitable Surface or Material

In step (C) of the present invention a suitable surface or material is provided. In preferred embodiments, a material consisting of a single type of atomic element is provided. As a non-limiting example, a carbon nanotube may be used as the material to find molecules with high affinity for carbon nanotubes. Methods of the present invention are suitable for identification of molecules which bind to fullerenes such as carbon nanotubes or graphene which consist of a single element (carbon). Methods of the present invention are also suitable for identification of molecules which binds to the surface of a metal such as gold, aluminum, lead, silver, or titanium. When using methods of the present invention to identify said molecules it is preferable that the surface comprises a single metal element such as gold (Au), lead (Pb), silver (Ag), or titanium (Ti).

Example materials consisting of only gold (Au) include the following: Gold nanotubes, gold nanowires. Example materials consisting of only titanium include the following: titanium rods, titanium plates. Example materials consisting of only silver include the following: silver fibres, silver cones. Example materials consisting of only zinc include the following: zinc nanotubes, zinc particles. Example materials consisting of only copper include the following: copper spheres, copper wires. On other preferred embodiments, the surface comprises two elements such as graphane which comprises carbon (C) and hydrogen (H). Example materials consisting of only zinc (Zn) and oxygen (O) include the following: ZnO nanotubes. Example materials consisting of only carbon (C) and hydrogen (H) include the following: polyethylene, polypropylene, polystyrene, graphane, Example materials consisting of two types of atoms include polytetrafluoroethylene (comprising C, F). Materials comprising C and O include graphene oxide. Materials comprising C and F include fluorinated graphene. Materials comprising C, H, Cl include polyvinylchloride. Other materials comprising 3 elements include poly(vinylalcohol) (C, H, O), polyacrylonitrile (C, H, N), poly[(dimethylsilylene)methylene] (C, H, Si), poly(thiophene) and thiol-functionalized graphene (C, H, S), hydroxide-functionalized graphene and hydroxide-functionalized carbon nanotubes (C, O, H), thiol-functionalized carbon nanotubes (C, S, H). Materials made up of 4 elements include poly(vinyl chloroacetate) (C, H, Cl, O), proteins and peptides (C, H, O, N). Materials made up of 5 elements include nucleic acids (C, H, N, O, P). A material may consist of just one atom (in its non-charged form or as an ion, e.g. Gd or Gd+++), or may consist of several atoms, held together in an organized structure. The following ions are particularly preferred materials: K+, Cl−, Ca++, Mg++, Gd+++, Cu+, Cu2+, Fe2+, Fe3+, Hg2+, Hg₂2+, Pb2+, Pb4+, Sn2+, Sn4+, Cr2+, Cr3+, Mn2+, Mn3+, Co2+, Co3+.

In cases where the transfer of a high number of library molecules in each round of the selection is the most important characteristic, a high concentration of material, such as CNTs, will be required. Thus, depending on the context, the concentration of the material, e.g CNTs, measured in mg/ml is preferably greater than 1E−9, such as greater than 1E−8, such as greater than 1E−7, such as greater than 1E−6, such as greater than 1E−5, such as greater than 1E−4, such as greater than 1E−3, such as greater than 1E−2, such as greater than 1E−1, such as greater than 1E+0, such as greater than 1E+1, such as greater than 1E+2, such as greater than 1E+3.

In cases where a high enrichment fold in each round of selection is the most important characteristic, a low concentration of material, e.g. CNTs, will be required. Thus, depending on the context, the concentration of material, such as CNTs, measured in mg/ml is preferably less than 1E+3, such as less than 1E+2, such as less than 1E+1, such as less than 1E+0, such as less than 1E−1, such as less than 1E−2, such as less than 1E−3, such as less than 1E−4, such as less than 1E−5, such as less than 1E−6, such as less than 1E−7, such as less than 1E−8, such as less than 1E−9.

### Molecular Weight (MW)

During screening, the weight of a material may be used to partition the material, e.g. with bound ligands, from non-bound molecules. This can be achieved, e.g. by centrifugation such that the material due to its weight will settle at the bottom of the centrifugated vial, whereas unbound ligand will stay in solution. Thus when a high weight is the most important characteristic of the material used, a high MW is preferred. Thus, depending on the context, the MW as measured in Dalton is preferably greater than 10, such as greater than 100, such as greater than 10², such as greater than 10³, such as greater than 10⁴, such as greater than 10⁵, such as greater than 10⁶, such as greater than 10⁸, such as greater than 10⁹. In cases where it is important to use a material with a low weight, e.g. so that the material stays in solution during centrifugation. Thus, when low weight is the most important characteristic of the material, a low MW is preferred. Thus, depending on context, the MW of library molecules measured in Dalton is preferably lower than 10⁹, such as lower than 10⁸, such as lower than 10⁷, such as lower than 10⁶, such as lower than 10⁵, such as lower than 10⁴, such as lower than 10³, such as lower than 10², such as lower than 10. In cases where both affinity and cost are important characteristics, the MW measured in Dalton is preferably; 10¹ to 10², or 10² to 10³, or 10³ to 10⁴, or 10⁴ to 10⁵, or 10⁵ to 10⁶, or 10⁶ to 10⁷, or 10⁷ to 10⁸, or 10⁸ to 10⁹.

### Density

For any characteristic of a material mentioned above, and in each characteristic's entire range, a further characteristic of importance is the density of the material. When ligands are sought for materials which a meant to be used as additives in composite materials of light weight, a low density is the most important characteristic of the material used. Thus, depending on the context, the density is preferably lower than 1E+20, such as lower than 1E+19, such as lower than 1E+18, such as lower than 1E+17, such as lower than 1E+16, such as lower than 1E+15, such as lower than 1E+14, such as lower than 1E+13, such as lower than 1E+12, such as lower than 1E+11, such as lower than 1E+10, such as lower than 1E+9, such as lower than 1E+8, such as lower than 1E+7, such as lower than 1E+6, such as lower than 1E+5, such as lower than 1E+4, such as lower than 1E+3, such as lower than 1E+2, such as lower than 1E+1, such as lower than 1E+0, such as lower than 1E−1, such as lower than 1E−2, such as lower than 1E−3, such as lower than 1E−4, such as lower than 1E−5, such as lower than 1E−6, such as lower than 1E−7, such as lower than 1E−8, such as lower than 1E−9, such as lower than 1E−10, such as lower than 1E−11, such as lower than 1E−12, such as lower than 1E−13, such as lower than 1E−14, such as lower than 1E−15, such as lower than 1E−16, such as lower than 1E−17, such as lower than 1E−18, such as lower than 1E−19, such as lower than 1E−20. When ligands are sought for materials which a meant to be used as additives in composite materials which efficiently can absorb sound or energy, a high density is the most important characteristic of the material used. Thus, depending on the context, the density of the material is preferably higher than 1E−20, such as more than 1E−19, such as more than 1E−18, such as more than 1E−17, such as more than 1E−16, such as more than 1E−15, such as more than 1E−14, such as more than 1E−13, such as more than 1E−12, such as more than 1E−11, such as more than 1E−10, such as more than 1E−9, such as more than 1E−8, such as more than 1E−7, such as more than 1E−6, such as more than 1E−5, such as more than 1E−4, such as more than 1E−3, such as more than 1E−2, such as more than 1E−1, such as more than 1E+0, such as more than 1E+1, such as more than 1E+2, such as more than 1E+3, such as more than 1E+4, such as more than 1E+5, such as more than 1E+6, such as more than 1E+7, such as more than 1E+8, such as more than 1E+9, such as more than 1E+10, such as more than 1E+11, such as more than 1E+12, such as more than 1E+13, such as more than 1E+14, such as more than 1E+15, such as more than 1E+16, such as more than 1E+17, such as more than 1E+18, such as more than 1E+19, such as more than 1E+20.

### Number of Different Elements

For any characteristic of a material mentioned above, and in each characteristic's entire range, a further characteristic of importance is the number of different elements of the material. The material preferably comprises a number of different atomic elements chosen from 1 to 2, 2 to 3, 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, 20 to 21, 21 to 22, 22 to 23, 23 to 24, 24 to 25 different atomic elements. In preferred embodiments, materials are composed of a single element, or two different elements, or three different elements, or four different elements, or five different elements, or six different elements, or seven different elements, or eight different elements, or nine different elements, or ten different elements, chosen from: Hydrogen (H), Helium (He), Lithium (Li), Beryllium (Be), Boron (B), Carbon (C), Nitrogen (N), Oxygen (O), Fluorine (F), Neon (Ne), Sodium (Na), Magnesium (Mg), Aluminium (Al), Silicon (Si), Phosphorus (P), Sulfur (S), Chlorine (Cl), Argon (Ar), Potassium (K), Calcium (Ca), Scandium (Sc), Titanium (Ti), Vanadium (V), Chromium (Cr), Manganese (Mn), Iron (Fe), Cobalt (Co), Nickel (Ni), Copper (Cu), Zinc (Zn), Gallium (Ga), Germanium (Ge), Arsenic (As), Selenium (Se), Bromine (Br), Krypton (Kr), Rubidium (Rb), Strontium (Sr), Yttrium (Y), Zirconium (Zr), Niobium (Nb), Molybdenum (Mo), Technetium (Tc), Ruthenium (Ru), Rhodium (Rh), Palladium (Pd), Silver (Ag), Cadmium (Cd), Indium (In), Tin (Sn), Antimony (Sb), Tellurium (Te), Iodine (I), Xenon (Xe), Caesium (Cs), Barium (Ba), Lanthanum (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Hafnium (Hf), Tantalum (Ta), Tungsten (W), Rhenium (Re), Osmium (Os), Iridium (Ir), Platinum (Pt), Gold (Au), Mercury (Hg), Thallium (Tl), Lead (Pb), Bismuth (Bi), Polonium (Po), Astatine (At), Radon (Rn), Francium (Fr), Radium (Ra), Actinium (Ac), Thorium (Th), Protactinium (Pa), Uranium (U), Neptunium (Np), Plutonium (Pu).

In a preferred embodiment, the material comprises a single atomic element such as carbon (for example fullerenes, carbon nanotubes, graphene). In a preferred embodiment, the material comprises two atomic elements such as carbon and hydrogen. In another preferred embodiment, the material comprises three atomic elements such as carbon, hydrogen and oxygen.

### Dielectric Constant

For any characteristic of a material mentioned above, and in each characteristic's entire range, a further characteristic of importance is the dielectric constant of the material. To efficiently identify hydrophobic ligands, a low dielectric constant is the most important characteristic of the material used. Thus, depending on the context, the dielectric constant of the medium is preferably lower than 250000, such as lower than 200000, such as lower than 50000, such as lower than 20000, such as lower than 1000, such as lower than 500, such as lower than 200, such as lower than 100, such as lower than 80, such as lower than 40, such as lower than 30, such as lower than 20, such as lower than 15, such as lower than 10, such as lower than 8, such as lower than 5, such as lower than 4, such as lower than 3, such as lower than 2, such as 1. To identify hydrophilic ligands, a high dielectric constant is the most important characteristic of the material used. Thus, depending on the context, the dielectric constant of the medium is preferably higher than 1, such as higher than 2, such as higher than 3, such as higher than 4, such as higher than 5, such as higher than 8, such as higher than 10, such as higher than 15, such as higher than 20, such as higher than 30, such as higher than 40, such as higher than 80, such as higher than 100, such as higher than 200, such as higher than 500, such as higher than 1000, such as higher than 20000, such as higher than 50000, such as higher than 200000, such as higher than 250000.

Young's Modulus

For any characteristic of a material mentioned above, and in each characteristic's entire range, a further characteristic of importance is the Young's modulus of the material. When ligands are sought for use in anchoring materials in a composite with a high Young's modulus is sought, a high Young's modulus of the material used for screening is the most important characteristic. Thus, depending on the context, the Young's modulus measured in megapascal is preferably higher than 1E−4, such as higher than 1E−3, such as higher than 1E−2, such as higher than 1E−1, such as higher than 1E+0, such as higher than 1E+1, such as higher than 1E+2, such as higher than 1E+3, such as higher than 1E+4, such as higher than 1E+5, such as higher than 1E+6. When ligands are sought for use in anchoring materials in a composite with a low Young's modulus is sought, a low Young's modulus of the material used for screening is the most important characteristic. Thus, depending on the context, the Young's modulus measured in megapascal is preferably lower than 1E+6, such as lower than 1E+5, such as lower than 1E+4, such as lower than 1E+3, such as lower than 1E+2, such as lower than 1E+1, such as lower than 1E+0, such as lower than 1E−1, such as lower than 1E−2, such as lower than 1E−3, such as lower than 1E−4.

### Tensile Strength

For any characteristic of a material mentioned above, and in each characteristic's entire range, a further characteristic of importance is the tensile strength of the material. When ligands are sought for use in anchoring materials in a composite with a high tensile strength is sought, a high tensile strength of the material used for screening is the most important characteristic. Thus, depending on the context, the tensile strength of the material used as measured in megapascal is preferably higher than 1E−4, such as higher than 1E−3, such as higher than 1E−2, such as higher than 1E−1, such as higher than 1E+0, such as higher than 1E+1, such as higher than 1E+2, such as higher than 1E+3, such as higher than 1E+4, such as higher than 1E+5, such as higher than 1E+6. When ligands are sought for use in anchoring materials in a composite with a low tensile strength is sought, a low tensile strength of the material used for screening is the most important characteristic. Thus, depending on the context, the tensile strength of the material used as measured in megapascal is preferably lower than 1E+6, such as lower than 1E+5, such as lower than 1E+4, such as lower than 1E+3, such as lower than 1E+2, such as lower than 1E+1, such as lower than 1E+0, such as lower than 1E−1, such as lower than 1E−2, such as lower than 1E−3, such as lower than 1E−4.

### Hydrogen Bond Acceptors (HBA)

For any characteristic of a material mentioned above, and in each characteristic's entire range, a further characteristic of importance is the number of HBAs of the material. In cases where ligands are sought with a high number of hydrogen bond donors, a high number of HBA is the most important characteristic of the material. Thus, depending on context, the number of HBA of the material surface per 100 square angstroms is preferably more than 1, such as more than 2, such as more than 3, such as more than 4, such as more than 5, such as more than 6, such as more than 7, such as more than 8, such as more than 9, such as more than 11, such as more than 13, such as more than 15, such as more than 17, such as more than 19, such as more than 21, such as more than 23, such as more than 25, such as more than 27, such as more than 29, such as more than 34, such as more than 39, such as more than 44, such as more than 49, such as more than 54, such as more than 59, such as more than 64, such as more than 69, such as more than 74, such as more than 79. In cases where ligands are sought with a low number of hydrogen bond donors, a low number of HBA is the most important characteristic of the material. Thus, depending on context, the number of HBA of the material surface per 100 square angstroms is preferably less than 94, such as less than 89, such as less than 84, such as less than 79, such as less than 74, such as less than 69, such as less than 64, such as less than 59, such as less than 54, such as less than 49, such as less than 44, such as less than 39, such as less than 34, such as less than 29, such as less than 27, such as less than 25, such as less than 23, such as less than 21, such as less than 19, such as less than 17, such as less than 15, such as less than 13, such as less than 11, such as less than 9, such as less than 8, such as less than 7, such as less than 6, such as less than 5, such as less than 4.

### Hydrogen Bond Donors (HBD)

For any characteristic of a material mentioned above, and in each characteristic's entire range, a further characteristic of importance is the number of HBDs of the material. In cases where ligands are sought with a high number of hydrogen bond acceptors, a high number of HBD is the most important characteristic of the material. Thus, depending on context, the number of HBD of the material surface per 100 square angstroms is preferably more than 1, such as more than 2, such as more than 3, such as more than 4, such as more than 5, such as more than 6, such as more than 7, such as more than 8, such as more than 9, such as more than 11, such as more than 13, such as more than 15, such as more than 17, such as more than 19, such as more than 21, such as more than 23, such as more than 25, such as more than 27, such as more than 29, such as more than 34, such as more than 39, such as more than 44, such as more than 49, such as more than 54, such as more than 59, such as more than 64, such as more than 69, such as more than 74, such as more than 79. In cases where ligands are sought with a low number of hydrogen bond acceptors, a low number of HBD is the most important characteristic of the material. Thus, depending on context, the number of HBD of the material surface per 100 square angstroms is preferably less than 94, such as less than 89, such as less than 84, such as less than 79, such as less than 74, such as less than 69, such as less than 64, such as less than 59, such as less than 54, such as less than 49, such as less than 44, such as less than 39, such as less than 34, such as less than 29, such as less than 27, such as less than 25, such as less than 23, such as less than 21, such as less than 19, such as less than 17, such as less than 15, such as less than 13, such as less than 11, such as less than 9, such as less than 8, such as less than 7, such as less than 6, such as less than 5, such as less than 4

### Charge

For any characteristic of a material mentioned above, and in each characteristic's entire range, a further characteristic of importance is the charge of the material. In cases where positively charged ligands are sought, a negative charge is the most important characteristic of the material used. Thus, depending on context, the charge of the material surface per 100 square angstrom is preferably higher than −50, such as higher than −45, such as higher than −40, such as higher than −35, such as higher than −30, such as higher than −25, such as higher than −20, such as higher than −15, such as higher than −10, such as higher than −5, such as higher than 0, such as higher than 5, such as higher than 10, such as higher than 15, such as higher than 20, such as higher than 25, such as higher than 30, such as higher than 35, such as higher than 40, such as higher than 45, such as higher than 50. In cases where negatively charged ligands are sought, a positive charge is the most important characteristic of the material used. Thus, depending on context, the charge of the material surface per 100 square angstrom is preferably lower than 50, such as lower than 45, such as lower than 40, such as lower than 35, such as lower than 30, such as lower than 25, such as lower than 20, such as lower than 15, such as lower than 10, such as lower than 5, such as lower than 0, such as lower than −5, such as lower than −10, such as lower than −15, such as lower than −20, such as lower than −25, such as lower than −30, such as lower than −35, such as lower than −40, such as lower than −45, such as lower than −50.

### Resistivity

For any characteristic of a surface or material mentioned above or below, and in each characteristic's entire range, the material preferably has a resistivity (measured in ohm metres) of 1E−10 to 1E−9, 1E−9 to 5E−8, 5E−8 to 2E−6, 2E−6 to 5E−5, 5E−5 to 1E−3, 1E−3 to 4E−2, 4E−2 to 1E+0, 1E+0 to 4E+1, 4E+1 to 1E+3, 1E+3 to 3E+4, 3E+4 to 1E+6, 1E+6 to 3E+7, 3E+7 to 9E+8, 9E+8 to 3E+10, 3E+10 to 8E+11, 8E+1l to 2E+13, 2E+13 to 7E+14, 7E+14 to 2E+16, 2E+16 to 6E+17, 6E+17 to 2E+19, 2E+19 to 6E+20, 6E+20 to 2E+22, 2E+22 to 5E+23, 5E+23 to 2E+25

### Materials

Preferred materials of the present invention include; a carbon fibre, a carbon nanofibre, a carbon nanothread, a ceramic material, a composite material, a fullerene, a MWCNT, a SWCNT, graphane, graphene oxide, graphite, graphite, graphyne, a COOH-functionalized carbon nanotube, a OH-functionalized carbon nanotube, an NH2-functionalized carbon nanotube, an SH-functionalized CNT, COOH-functionalized graphene, multi-layer graphene, NH2-functionalized graphene, OH-functionalized graphene, reduced graphene oxide, thiol-functionalized graphene, a glass fibre, aramid, E-glass, iron, polyester, polyethylene, S-glass, steel, a battery, a borosilicate, a buckyball, a buckytube, a capacitator, a carbon dome, a carbon material, a carbon megatube, a carbon nanofoam, a carbon polymer, a catalyst, a cathode, a coated carbon nanotube, a conductor, a covalent crystal, a crystal, a crystalline material, a defect-free graphene sheet, a defect-free MWCNT, a defect-free SWCNT, a dielectric material, a diode, a dodecahedrane, a doped glass, a fibre, a fullerite, a fused silica, a glue, a green ceramic, a lanthanides, a machinable ceramic, a metal alloy, a metal-functionalized carbon nanotube, a metalised dielectric, a metallised ceramic, a metalloid, a mineral, a non-covalent crystal, a piezoelectric material, a platinum group metal, a post-transition metal, a rare earth element, a sapphire, a semiconductor, a sensor, a silicon nitride, a single crystal fiber, a sol-gel, a synthetic diamond, a transition metal, a triple-wall carbon nanotube, a tungsten carbide, alumina, alumina trihydrate, aluminium, aluminum boride, aluminum oxide, aluminum trihydroxide, amorphous carbon, an actinides, an amalgam, an anode, an elastomers, an electrode, an endohedral fullerene, an insulator, an intermetallic, an ionic crystal, an organic material, anode, anthracite, asbestos, barium, bone, boron, brass, buckypaper, calcium carbonite, calcium metasilicate, calcium sulfate, calcium sulphate, carbon black, carbon nanofoam, cathode, chromium, clay, coal, copper, diamond, diamond-like carbon, double-layer graphene, exfoliated graphite, exfoliated silicate, flourinated graphene, fused silica, gallium arsenide, gallium nitride, germanium, glass, glass microsphere, glass ribbons, glassy carbon, gold, hardened steel, hydrous magnesium silicate, hyperdiamond, iron oxides, lead zirconium titanate, lignite, lithium niobate, lonsdaleite, magnesium dihydroxide, magnesium oxide, manganese, metal, metal oxide, mica, molybdenum, nickel, nylon, palladium, pencil lead, platinum, prismane, pyrolytic graphite, rubber, silica, silica gel, silicon, silicon carbide, silicon dioxide, silicon nitride, silver, soot, stainless steel, tantalum, titanium, titanium oxide, tooth cementum, tooth dentine, tooth enamel, tungsten, tungsten carbide, wood, zinc oxide, zirconia.

### Chiral Vectors

A characteristic of some materials is their chiral vector. For example the carbon nanotube can be conceptualized by wrapping a one-atom-thick layer of graphite called graphene into a seamless cylinder. The way the graphene sheet is wrapped is represented by a pair of indices (n,m) called the chiral vector. The integers n and m denote the number of unit vectors along two directions in the honeycomb crystal lattice of graphene. If m=0, the nanotubes are called zigzag nanotubes, and if n=m, the nanotubes are called armchair nanotubes. Otherwise, they are called chiral. As non-limiting examples, carbon nanotubes are characterised by a chiral vector (n, m) selected from: (0,0); (1,0); (2,0); (3,0); (4,0); (5,0); (6,0); (7,0); (8,0); (9,0); (10,0); (11,0); (12,0); (13,0); (14,0); (15,0); (16,0); (17,0); (18,0); (19,0); (20,0); (0,1); (1,1); (2,1); (3,1); (4,1); (5,1); (6,1); (7,1); (8,1); (9,1); (10,1); (11,1); (12,1); (13,1); (14,1); (15,1); (16,1); (17,1); (18,1); (19,1); (20,1); (0,2); (1,2); (2,2); (3,2); (4,2); (5,2); (6,2); (7,2); (8,2); (9,2); (10,2); (11,2); (12,2); (13,2); (14,2); (15,2); (16,2); (17,2); (18,2); (19,2); (20,2); (0,3); (1,3); (2,3); (3,3); (4,3); (5,3); (6,3); (7,3); (8,3); (9,3); (10,3); (11,3); (12,3); (13,3); (14,3); (15,3); (16,3); (17,3); (18,3); (19,3); (20,3); (0,4); (1,4); (2,4); (3,4); (4,4); (5,4); (6,4); (7,4); (8,4); (9,4); (10,4); (11,4); (12,4); (13,4); (14,4); (15,4); (16,4); (17,4); (18,4); (19,4); (20,4); (0,5); (1,5); (2,5); (3,5); (4,5); (5,5); (6,5); (7,5); (8,5); (9,5); (10,5); (11,5); (12,5); (13,5); (14,5); (15,5); (16,5); (17,5); (18,5); (19,5); (20,5); (0,6); (1,6); (2,6); (3,6); (4,6); (5,6); (6,6); (7,6); (8,6); (9,6); (10,6); (11,6); (12,6); (13,6); (14,6); (15,6); (16,6); (17,6); (18,6); (19,6); (20,6); (0,7); (1,7); (2,7); (3,7); (4,7); (5,7); (6,7); (7,7); (8,7); (9,7); (10,7); (11,7); (12,7); (13,7); (14,7); (15,7); (16,7); (17,7); (18,7); (19,7); (20,7); (0,8); (1,8); (2,8); (3,8); (4,8); (5,8); (6,8); (7,8); (8,8); (9,8); (10,8); (11,8); (12,8); (13,8); (14,8); (15,8); (16,8); (17,8); (18,8); (19,8); (20,8); (0,9); (1,9); (2,9); (3,9); (4,9); (5,9); (6,9); (7,9); (8,9); (9,9); (10,9); (11,9); (12,9); (13,9); (14,9); (15,9); (16,9); (17,9); (18,9); (19,9); (20,9); (0,10); (1,10); (2,10); (3,10); (4,10); (5,10); (6,10); (7,10); (8,10); (9,10); (10,10); (11,10); (12,10); (13,10); (14,10); (15,10); (16,10); (17,10); (18,10); (19,10) (20,10)(0,11); (1,11); (2,11); (3,11); (4,11); (5,11); (6,11); (7,11); (8,11); (9,11); (10,11); (11,11); (12,11); (13,11); (14,11); (15,11); (16,11); (17,11); (18,11); (19,11); (20,11); (0,12); (1,12); (2,12); (3,12); (4,12); (5,12); (6,12); (7,12); (8,12); (9,12); (10,12); (11,12); (12,12); (13,12); (14,12); (15,12); (16,12); (17,12); (18,12); (19,12); (20,12); (0,13); (1,13); (2,13); (3,13); (4,13); (5,13); (6,13); (7,13); (8,13); (9,13); (10,13); (11,13); (12,13); (13,13); (14,13); (15,13); (16,13); (17,13); (18,13); (19,13); (20,13); (0,14); (1,14); (2,14); (3,14); (4,14); (5,14); (6,14); (7,14); (8,14); (9,14); (10,14); (11,14); (12,14); (13,14); (14,14); (15,14); (16,14); (17,14); (18,14); (19,14); (20,14); (0,15); (1,15); (2,15); (3,15); (4,15); (5,15); (6,15); (7,15); (8,15); (9,15); (10,15); (11,15); (12,15); (13,15); (14,15); (15,15); (16,15); (17,15); (18,15); (19,15); (20,15); (0,16); (1,16); (2,16); (3,16); (4,16); (5,16); (6,16); (7,16); (8,16); (9,16); (10,16); (11,16); (12,16); (13,16); (14,16); (15,16); (16,16); (17,16); (18,16); (19,16); (20,16); (0,17); (1,17); (2,17); (3,17); (4,17); (5,17); (6,17); (7,17); (8,17); (9,17); (10,17); (11,17); (12,17); (13,17); (14,17); (15,17); (16,17); (17,17); (18,17); (19,17); (20,17); (0,18); (1,18); (2,18); (3,18); (4,18); (5,18); (6,18); (7,18); (8,18); (9,18); (10,18); (11,18); (12,18); (13,18); (14,18); (15,18); (16,18); (17,18); (18,18); (19,18); (20,18); (0,19); (1,19); (2,19); (3,19); (4,19); (5,19); (6,19); (7,19); (8,19); (9,19); (10,19); (11,19); (12,19); (13,19); (14,19); (15,19); (16,19); (17,19); (18,19); (19,19); (20,19); (0,20); (1,20); (2,20); (3,20); (4,20); (5,20); (6,20); (7,20); (8,20); (9,20); (10,20); (11,20); (12,20); (13,20); (14,20); (15,20); (16,20); (17,20); (18,20); (19,20); (20,20).

### Materials Structure

In preferred embodiments, materials have a structure chosen from; dots such as a quantum dot; colloidals such as colloidal gold; a cup, such as a nanocup, a stacked carbon nanocup; a particle, such as a nanoparticle, a silver nanoparticle or a gold nanoparticle, a microparticle; a tube, such as a carbon tube, a metallic tube; a nanotube, such as a metallic nanotube, a nanotube, nanotubes made of carbon, zinc, gold, silver, aluminum, boron; a microtube; a film; a fibre, a carbon fibre, such as a vitreous fibre, such as a glass fibre; a metallic fibre, such as a metallic carbon nanofibre; a nanofibre, a microfibre, a ribbon, such as a nanoribbon, carbon nanoribbon; a microribbon; a layer, such as a monolayer, fullerenere monolayer; a multilayer, such as a fullerene multilayer, a sphere, such as a nanosphere, a sheet, such as a nanosheet, a foam; a rod, such as a nanorod; an aggregated diamond nanorod; a knot; a wall, such as a carbon nanowall; a nanoparticles, such as silver nanoparticles, iron nanoparticles, platinum nanoparticles; a nanotube made from ZnO, CeO2, CNT or gold; a particle; a surface; a nanocluster; a carbon nanofiber, a diamond, a diamond film; a bamboo-structured nanotube; a filter, a pore; a membrane, such as a nanomembrane.

### Defect Surfaces

In preferred embodiments, defect surfaces or materials are used. For example, a material suitable for the present invention is iron. Pure iron may be considered defect-free and partly oxidized iron may be considered defect in that each oxidated atomic position corresponds to a defect. In preferred embodiments, defect surfaces or materials are used to identify molecules which bind with high affinity to defect sites. In other preferred embodiments defect-free surfaces are used to identify molecules with high affinity for defect-free surfaces or areas of surfaces which are defect-free. A defect-free surface may for example be defect-free graphene nanoribbons (Graphene Nanoribbons by Chemists: Nanometer-Sized, Soluble, and Defect-Free, Dossel et al. Angewandte Chemie International Edition, DOI: 10.1002/anie.201006593). In a preferred embodiment, carbon nanotubes with defects comprising sp3-hybridized carbon atoms are used as a material. In another preferred embodiment, graphene with defects comprising sp3-hybridized carbon atoms is used as a material. In preferred embodiments, the number of defects per square nanometer surface or material is 0, 0-1, 1-2, 2-3, 3-5, 5-10, 10-100, 100-1000, 10000 to 100000. In other preferred embodiments, the number of defects per 1000 atoms surface or material is 0, 0-1, 1-2, 2-3, 3-5, 5-10, 10-100, 100-1000. In yet other preferred embodiments, the number of defects per cubic nanometer surface or material is 0, 0-1, 1-2, 2-3, 3-5, 5-10, 10-100, 100-1000, 10000 to 100000. In yet another preferred embodiment, a defect (mutant) protein is used as material.

### Inorganic Materials

In preferred embodiments, the material comprises one or more entities chosen from: Aluminium antimonide—AlSb, Aluminium arsenide—AlAs, Aluminium chloride—AlCl3, Aluminium fluoride—AlF3, Aluminium hydroxide—Al(OH)3, Aluminium nitrate—Al(NO3)3, Aluminium nitride—AlN, Aluminium oxide—Al2O3, Aluminium phosphide—AlP, Aluminium sulfate—Al2(SO4)3, Ammonia—NH3, Ammonium bicarbonate—NH4HCO3, Ammonium cerium(IV) nitrate—(NH4)2Ce(NO3)6, Ammonium chloride—NH4Cl, Ammonium hydroxide—NH4OH, Ammonium nitrate—NH4NO3, Ammonium sulfate—(NH4)2SO4, Ammonium tetrathiocyanatodiamminechromate(III)—NH4[Cr(SCN)4(NH3)2], Antimony hydride—SbH3, Antimony pentachloride—SbCl5, Antimony pentafluoride—SbF5, Antimony trioxide—Sb2O3, Arsenic trioxide (Arsenic(III) oxide)—As2O3, Arsenous acid—As(OH)3, Arsine—AsH3, Baking soda—NaHCO3, Barium chloride—BaCl2, Barium chromate—BaCrO4, Barium hydroxide—Ba(OH)2, Barium iodide—BaI2, Barium nitrate—Ba(NO3)2, Barium sulfate—BaSO4, Barium titanate—BaTiO3, Beryllium borohydride—Be(BH4)2, Beryllium bromide—BeBr2, Beryllium carbonate—BeCO3, Beryllium chloride—BeCl2, Beryllium fluoride—BeF2, Beryllium hydride—BeH2, Beryllium hydroxide—Be(OH)2, Beryllium iodide—BeI2, Beryllium nitrate—Be(NO3)2, Beryllium nitride—Be3N2, Beryllium oxide—BeO, Beryllium sulfate—BeSO4, Beryllium sulfite—BeSO3, Beryllium telluride—BeTe, Bismuth(III) oxide—Bi2O3, Bismuth(III) telluride—Bi2Te3, Borane—Diborane: B2H6, Pentaborane: B5H9 Decaborane: B10H14, Borax—Na2B4O7.10H2O, Boric acid—H3BO3, Boron carbide—B4C, Boron nitride—BN, Boron oxide—B2O3, Boron suboxide—B6O, Boron trichloride—BCl3, Boron trifluoride—BF3, Bromine pentafluoride—BrF5, Bromine trifluoride—BrF3, Cacodylic acid—(CH3)2AsO2H, Cadmium arsenide—Cd3As2, Cadmium bromide—CdBr2, Cadmium chloride—CdCl2, Cadmium fluoride—CdF2, Cadmium iodide—CdI2, Cadmium nitrate—Cd(NO3)2, Cadmium selenide—CdSe (of quantum dot fame), Cadmium sulfate—CdSO4, Cadmium telluride—CdTe, Caesium bicarbonate—CsHCO3, Caesium carbonate—Cs2CO3, Caesium chloride—CsCl, Caesium chromate—Cs2CrO4, Caesium fluoride—CsF, Caesium hydride—CsH, Calcium carbide—CaC2, Calcium chlorate—Ca(ClO3)2, Calcium chloride—CaCl2, Calcium chromate—CaCrO4, Calcium cyanamide—CaCN2, Calcium fluoride—CaF2, Calcium hydride—CaH2, Calcium hydroxide—Ca(OH)2, Calcium sulfate (Gypsum)—CaSO4, Carbon dioxide—CO2, Carbon disulfide—CS2, Carbon monoxide—CO, Carbon tetrabromide—CBr4, Carbon tetrachloride—CCl4, Carbon tetraiodide—CI4, Carbonic acid—H2CO3, Carbonyl fluoride—COF2, Carbonyl sulfide—COS, Carboplatin—C6H12N2O4Pt, carborundum SiC, Cerium aluminium—CeAl, Cerium cadmium—CeCd, Cerium magnesium—CeMg, Cerium mercury—CeHg, Cerium silver—CeAg, Cerium thallium—CeTl, Cerium zinc—CeZn, Cerium(III) bromide—CeBr3, Cerium(III) chloride—CeCl3, Cerium(IV) sulfate—Ce(SO4)2, Chrome-alum; K2SO4Cr2(SO4)3.24H2O, Chromic acid—CrO3, Chromium trioxide (Chromic acid)—CrO3, Chromium(II) chloride—CrCl2 (also chromous chloride), Chromium(II) sulfate—CrSO4, Chromium(III) chloride—CrCl3, Chromium(III) oxide—Cr2O3, Chromium(IV) oxide—CrO2, Chromyl chloride—CrO2Cl2, Cisplatin (cis-platinum(II) chloride diammine)—PtCl2(NH3)2, Cobalt(II) bromide—CoBr2, Cobalt(II) carbonate—CoCO3, Cobalt(II) chloride—CoCl2, Cobalt(II) sulfate—CoSO4, Columbite—Fe2+Nb2O6, Copper(I) chloride—CuCl, Copper(I) oxide—Cu2O, Copper(I) sulfide—Cu2S, Copper(II) carbonate—CuCO3, Copper(II) chloride—CuCl2, Copper(II) hydroxide—Cu(OH)2, Copper(II) nitrate—Cu(NO3)2, Copper(II) oxide—CuO, Copper(II) sulfate—CuSO4, Copper(II) sulfide—CuS, Cyanogen—(CN)2, Cyanogen chloride—CNCl, Cyanuric chloride—C3Cl3N3, Decaborane (Diborane)—B10H14, Diammonium phosphate—(NH4)2HPO4, Diborane—B2H6, Dichlorosilane—SiH2Cl2, Digallane—Ga2H6, Dinitrogen pentoxide (nitronium nitrate)—N2O5, Disilane—Si2H6, Disulfur dichloride S2Cl2, Dysprosium(III) chloride—DyCl3, Erbium(III) chloride—ErCl3, Erbium-copper—ErCu, Erbium-gold—ErAu, Erbium-Iridium—ErIr, Erbium-silver—ErAg, Europium(III) chloride—EuCl3, Fluorosulfuric acid—FSO2(OH), Gadolinium(III) chloride—GdCl3, Gadolinium(III) oxide—Gd2O3, Gallium antimonide—GaSb, Gallium arsenide—GaAs, Gallium nitride—GaN, Gallium phosphide—GaP, Gallium trichloride—GaCl3, Germanium (IV) nitride—Ge3N4, Germanium telluride—GeTe, Germanium(II) bromide—GeBr2, Germanium(II) chloride—GeCl2, Germanium(II) fluoride—GeF2, Germanium(II) iodide—GeI2, Germanium(II) oxide—GeO, Germanium(II) selenide—GeSe, Germanium(II) sulfide—GeS, Germanium(III) hydride—Ge2H6, Germanium(IV) bromide—GeBr4, Germanium(IV) chloride—GeCl4, Germanium(IV) fluoride—GeF4, Germanium(IV) hydride (Germane)—GeH4, Germanium(IV) iodide—GeI4, Germanium(IV) oxide—GeO2, Germanium(IV) selenide—GeSe2, Germanium(IV) sulfide—GeS2, Gold ditelluride—AuTe2, Gold(I) bromide—AuBr, Gold(I) chloride—AuCl, Gold(I) iodide—AuI, Gold(I) sulfide—Au2S, Gold(I,III) chloride—Au4Cl8, Gold(III) bromide—(AuBr3)2, Gold(III) chloride—(AuCl3)2, Gold(III) chloride—AuCl3, Gold(III) fluoride—AuF3, Gold(III) iodide—AuI3, Gold(III) oxide—Au2O3, Gold(III) selenide—Au2Se3, Gold(III) selenide—AuSe, Gold(III) sulfide—Au2S3, Gold(V) fluoride—AuF5, Hafnium fluoride, Hafnium tetrachloride—HfCl4, Hexadecacarbonylhexarhodium—Rh6CO16, Hydrazine—N2H4, Hydrazoic acid—HN3, Hydrobromic acid—HBr, Hydrochloric acid—HCl, Hydrogen bromide—HBr, Hydrogen chloride—HCl, Hydrogen fluoride—HF, Hydrogen peroxide—H2O2, Hydrogen selenide—H2Se, Hydrogen sulfide—H2S, Hydrogen telluride—H2Te, Hydroiodic acid—HI, Hydroxylamine—NH2OH, Hypochlorous acid—HClO, Hypophosphorous acid—H3PO2, Indium antimonide—InSb, Indium arsenide—InAs, Indium nitride—InN, Indium phosphide—InP, Indium(I) chloride, Iodic acid—HIO3, Iodine heptafluoride—IF7, Iodine monochloride—ICl, Iodine pentafluoride—IF5, Iridium(IV) chloride, Iron(II) chloride—FeCl2 including hydrate, Iron(II) oxide—FeO, Iron(II,III) oxide—Fe3O4, Iron(III) chloride—FeCl3, Iron(III) nitrate—Fe(NO3)3(H2O)9, Iron(III) oxide—Fe2O3, Iron(III) thiocyanate, Iron-sulfur cluster, Krypton difluoride—KrF2, Lanthanum aluminium—LaAl, Lanthanum cadmium—LaCd, Lanthanum carbonate—La2(CO3)3, Lanthanum magnesium—LaMg, Lanthanum mercury—LaHg, Lanthanum silver—LaAg, Lanthanum tallium—LaTl, Lanthanum zinc—LaZn, Lead zirconate titanate—Pb[TixZr1-x]O3 (e.g., x=0.52 is Lead zirconium titanate), Lead(II) carbonate—Pb(CO3), Lead(II) chloride—PbCl2, Lead(II) iodide—PbI2, Lead(II) nitrate—Pb(NO3)2, Lead(II) oxide—PbO, Lead(II) phosphate—Pb3(PO4)2, Lead(II) selenide—PbSe, Lead(II) sulfate—Pb(SO4), Lead(II) sulfide—PbS, Lead(II) telluride—PbTe, Lead(IV) oxide—PbO2, Lithium aluminium hydride—LiAlH4, Lithium bromide—LiBr, Lithium carbonate (Lithium salt)—Li2CO3, Lithium chloride—LiCl, Lithium hydride—LiH, Lithium hydroxide—LiOH, Lithium iodide—LiI, Lithium nitrate—LiNO3, Lithium sulfate—Li2SO4, Magnesium antimonide—MgSb, Magnesium carbonate—MgCO3, Magnesium chloride—MgCl2, Magnesium oxide—MgO, Magnesium phosphate—Mg3(PO4)2, Magnesium sulfate—MgSO4, Manganese(II) chloride—MnCl2, Manganese(II) phosphate—Mn3(PO4)2, Manganese(II) sulfate monohydrate—MnSO4.H2O, Manganese(III) chloride—MnCl3, Manganese(IV) fluoride—MnF4, Manganese(IV) oxide (manganese dioxide)—MnO2, Mercury fulminate—Hg(ONC)2, Mercury(I) chloride—Hg2Cl2, Mercury(I) sulfate—Hg2SO4, Mercury(II) chloride—HgCl2, Mercury(II) selenide—HgSe, Mercury(II) sulfate—HgSO4, Mercury(II) sulfide—HgS, Mercury(II) telluride—HgTe, Metaphosphoric acid—HPO3, Molybdate orange, Molybdenum disulfide—MoS2, Molybdenum hexacarbonyl—C6O6Mo, Molybdenum trioxide—MoO3, Molybdic acid—H2MoO4, Neodymium(III) chloride—NdCl3, Nessler's reagent—K2[HgI4], Nickel(II) carbonate—NiCO3, Nickel(II) chloride—NiCl2 and hexahydrate, Nickel(II) hydroxide—Ni(OH)2, Nickel(II) nitrate—Ni(NO3)2, Nickel(II) oxide—NiO, Niobium oxychloride—NbOCl3, Niobium pentachloride—NbCl5, Nitric acid—HNO3, Nitrogen dioxide—NO2, Nitrogen monoxide—NO, Nitrosylsulfuric acid—NOHSO4, Osmium tetroxide (osmium(VIII) oxide)—OsO4, Osmium trioxide (osmium(VI) oxide)—OsO3, Oxybis(tributyltin)—C24H54OSn2, Oxygen difluoride—OF2 Ozone—O3, Palladium(II) chloride—PdCl2, Palladium(II) nitrate—Pd(NO3)2, Pentaborane—B5H9, Pentasulfide antimony—Sb2S5, Perchloric acid—HClO4, Perchloryl fluoride—ClFO3, Persulfuric acid (Caro's acid)—H2SO5, Perxenic acid—H4XeO6, Phenylarsine oxide—(C6H5)AsO, Phenylphosphine—C6H7P, Phosgene—COCl2, Phosphine—PH3, Phosphite—HPO32-, Phosphomolybdic acid—HMoNiO6P-4, Phosphoric acid—H3PO4, Phosphorous acid (Phosphoric(III) acid)—H3PO3, Phosphorus pentabromide—PBr5, Phosphorus pentafluoride—PF5, Phosphorus pentasulfide—P4S10, Phosphorus pentoxide—P205, Phosphorus sesquisulfide—P4S3, Phosphorus tribromide—PBr3, Phosphorus trichloride—PCl3, Phosphorus trifluoride—PF3, Phosphorus triiodide—PI3, Phosphotungstic acid—H3PW12O40, Platinum(II) chloride—PtCl2, Platinum(IV) chloride—PtCl4, Plutonium dioxide (Plutonium(IV) oxide)—PuO2, Plutonium(III) chloride—PuCl3, Potash Alum—K2SO4.Al2(SO4)3.24H2O, Potassium aluminium fluoride—KAlF4, Potassium borate—K2B4O7.4H2O, Potassium bromide—KBr, Potassium calcium chloride—KCaCl3, Potassium carbonate—K2CO3, Potassium chlorate—KClO3, Potassium chloride—KCl, Potassium ferrioxalate—K3[Fe(C2O4)3], Potassium hydrogen fluoride—HF2K, Potassium hydrogencarbonate—KHCO3, Potassium hydroxide—KOH, Potassium iodide—KI, Potassium monopersulfate—K2SO4-KHSO4-2KHSO5, Potassium nitrate—KNO3, Potassium perbromate—KBrO4, Potassium perchlorate—KClO4, Potassium permanganate—KMnO4, Potassium sulfate—K2SO4, Potassium sulfide—K2S, Potassium titanyl phosphate—KTiOPO4, Potassium vanadate—KVO3, Praseodymium(III) chloride—PrCl3, Protonated molecular hydrogen—H3+, Prussian blue (Iron(III) hexacyanoferrate(II))—Fe4[Fe(CN)6]3, Pyrosulfuric acid—H2S2O7, Radium chloride—RaCl2, Radon difluoride—RnF2, Rhodium(III) chloride—RhCl3, Rubidium bromide—RbBr, Rubidium chloride—RbCl, Rubidium fluoride—RbF, Rubidium hydroxide—RbOH, Rubidium iodide—RbI, Rubidium nitrate—RbNO3, Rubidium oxide—Rb2O, Rubidium telluride—Rb2Te, Ruthenium(VIII) oxide—RuO4, Samarium(II) iodide—SmI2, Samarium(III) chloride—SmCl3, Scandium(III) chloride—ScCl3 and hydrate, Scandium(III) fluoride—ScF3, Scandium(III) nitrate—Sc(NO3)3, Scandium(III) oxide—Sc2O3, Scandium(III) triflate—Sc(OSO2CF3)3, Selenic acid—H2SeO4, Selenious acid—H2SeO3, Selenium dioxide—SeO2, Selenium trioxide—SeO3, Silane—SiH4, Silica gel—SiO2-nH2O, Silicic acid—[SiOx(OH)4−2x]n, Silicochloroform—C13HSi, Silicofluoric acid—H2SiF6, Silicon dioxide—SiO2, Silver chloride—AgCl, Silver iodide—AgI, Silver nitrate—AgNO3, Silver sulfide—Ag2S, Silver(I) fluoride—AgF, Silver(II) fluoride—AgF2, Soda lime—, Sodamide—NaNH2, Sodium borohydride—NaBH4, Sodium bromate—NaBrO3, Sodium bromide—NaBr, Sodium carbonate—Na2CO3, Sodium chlorate—NaClO3, Sodium chloride—NaCl, Sodium cyanide—NaCN, Sodium ferrocyanide—Na4Fe(CN)6, Sodium hydride—NaH, Sodium hydrogen carbonate (Sodium bicarbonate)—NaHCO3, Sodium hydrosulfide—NaSH, Sodium hydroxide—NaOH, Sodium iodide—NaI, Sodium monofluorophosphate (MFP)—Na2PFO3, Sodium nitrate—NaNO3, Sodium nitrite—NaNO2, Sodium percarbonate—2Na2CO3.3H2O2, Sodium persulfate—Na2S2O8, Sodium phosphate; see Trisodium phosphate—Na3PO4, Sodium silicate—Na2SiO3, Sodium sulfate—Na2SO4, Sodium sulfide—Na2S, Sodium sulfite—Na2SO3, Sodium tellurite—Na2TeO3, Stannous chloride (tin(II) chloride)—SnCl2, Stibine—SbH3, Strontium chloride—SrCl2, Strontium nitrate—Sr(NO3)2, Strontium titanate—SrTiO3, Sulfamic acid—H3NO3S, Sulfane—H2S, Sulfur dioxide—SO2, Sulfuric acid—H2SO4, Sulfurous acid—H2SO3, Sulfuryl chloride—SO2Cl2, Tantalum carbide—TaC, Tantalum(V) oxide—Ta2O5, Telluric acid—H6TeO6, Tellurium dioxide—TeO2, Tellurium tetrachloride—TeCl4, Tellurous acid—H2TeO3, Terbium(III) chloride—TbCl3, Tetraborane(10)—B4H10, Tetrachloroauric acid—AuCl3, Tetrafluorohydrazine—N2F4, Tetramminecopper(II) sulfate—[Cu(NH3)4]SO4, Tetrasulfur tetranitride—S4N4, Thallium(I) carbonate—Tl2CO3, Thallium(I) fluoride—TlF, Thallium(III) oxide T1203, Thallium(III) sulfate, Thionyl chloride—SOCl2, Thiophosgene—CSCl2, Thiophosphoryl chloride—Cl3PS, Thorium dioxide—ThO2, Thortveitite—(Sc,Y)2Si2O7, Thulium(III) chloride—TmCl3, Tin(II) chloride—SnCl2, Tin(II) fluoride—SnF2, Tin(IV) chloride—SnCl4, Titanium boride—TiB2, Titanium carbide—TiC, Titanium dioxide (B) (titanium(IV) oxide)—TiO2, Titanium dioxide (titanium(IV) oxide)—TiO2, Titanium nitride—TiN, Titanium(II) chloride—TiCl2, Titanium(III) chloride—TiCl3, Titanium(IV) bromide (titanium tetrabromide)—TiBr4, Titanium(IV) chloride (titanium tetrachloride)—TiCl4, Titanium(IV) iodide (titanium tetraiodide)—TiI4, Trifluoromethanesulfonic acid—CF3SO3H, Trifluoromethylisocyanide—C2NF3, Trimethylphosphine—C3H9P, Trioxidane—H2O3, Tripotassium phosphate—K3PO4, Trisodium phosphate—Na3PO4, Triuranium octaoxide (pitchblende or yellowcake)—U308, Tungsten carbide—WC, Tungsten hexacarbonyl—W(CO)6, Tungsten(VI) chloride—WCl6, Tungsten(VI) Fluoride—WF6, Tungstic acid—H2WO4, Uranium hexafluoride—UF6, Uranium pentafluoride—UF5, Uranium tetrachloride—UCl4, Uranium tetrafluoride—UF4, Uranyl carbonate—UO2CO3, Uranyl chloride—UO2Cl2, Uranyl fluoride—UO2F2, Uranyl hydroxide—(UO2)2(OH)4, Uranyl hydroxide—UO2(OH)2, Uranyl nitrate—UO2(NO3)2, Uranyl sulfate—UO2SO4, Vanadium carbide—VC, Vanadium oxytrichloride (Vanadium(V) oxide trichloride)—VOCl3, Vanadium(II) chloride—VCl2, Vanadium(II) oxide—VO, Vanadium(III) bromide—VBr3, Vanadium(III) chloride—VCl3, Vanadium(III) fluoride—VF3, Vanadium(III) nitride—VN, Vanadium(III) oxide—V2O3, Vanadium(IV) chloride—VCl4, Vanadium(IV) fluoride—VF4, Vanadium(IV) oxide—VO2, Vanadium(IV) sulfate—VOSO4 Vanadium(V) oxide—V2O5, Water—H2O, Xenic acid—H2XeO4, Xenon difluoride—XeF2, Xenon hexafluoroplatinate—Xe[PtF6], Xenon tetrafluoride—XeF4, Xenon tetroxide—XeO4, Ytterbium(III) chloride—YbCl3, Ytterbium(III) oxide—Yb2O3, Yttrium aluminium garnet—Y3Al5O12, Yttrium barium copper oxide—YBa2Cu3O7, Yttrium cadmium—YCd, Yttrium copper—YCu, Yttrium gold—YAu, Yttrium iridium—YIr, Yttrium iron garnet—Y3Fe5O12, Yttrium magnesium—YMg, Yttrium rhodium—YRh, Yttrium silver—YAg, Yttrium zinc—YZn, Yttrium(III) antimonide—YSb, Yttrium(III) arsenide—YAs, Yttrium(III) bromide—YBr3, Yttrium(III) fluoride—YF3, Yttrium(III) oxide—Y2O3, Yttrium(III) sulfide—Y2S3, Zinc bromide—ZnBr2, Zinc carbonate—ZnCO3, Zinc chloride—ZnCl2, Zinc cyanide—Zn(CN)2, Zinc fluoride—ZnF2, Zinc iodide—ZnI2, Zinc oxide—ZnO, Zinc selenide—ZnSe, Zinc sulfate—ZnSO4, Zinc sulfide—ZnS, Zinc telluride—ZnTe, Zirconia hydrate—ZrO2.nH2O, Zirconium carbide—ZrC, Zirconium hydroxide—Zr(OH)4, Zirconium nitride—ZrN, Zirconium orthosilicate—ZrSiO4, Zirconium tetrahydroxide—H4O4Zr, Zirconium tungstate—ZrW2O8, Zirconium(IV) chloride—ZrCl4, Zirconium(IV) oxide—ZrO2.

### Composite Materials

In preferred embodiments, materials are chosen from;

a carbon rubber composite, Carbon fibre composite, a carbon tube composite, a carbon nanotube composite. Carbon nanotube composites, a composite comprising Single-walled carbon nanotubes, Multi-walled carbon nanotubes, Functionalized carbon nanotubes, Defect-free carbon nanotubes, Defect carbon nanotubes, carbon nanotubes on silicon wafers, carbon nanotubes that contain gadolinium, Carbon nanohorns, Carbon nanobuds, Graphene, such as single layer graphene, multi-layer graphene, defect-free graphene, defect graphene, functionalized graphene, graphite. In a preferred embodiment, the material is a composite or a composite of composites.
Steps D, E, F, G, H; Isolating and/or Identifying Molecules with Desired Characteristics; Screening

To isolate molecules with desired characteristics, a molecule library is “screened” or subjected to selection by performing a series of steps.

Said steps are described in more detail in the following.

Step D. Combining One or More of Said Library, Medium, Surface and/or Material

In a step of the present invention, one or more molecules are provided, e.g., as a library of molecules and said molecules are combined with a surface or a material. In preferred embodiments, the molecules and surface/material is combined in the presence of a medium such as a liquid medium.

Step E. Optionally Performing One or More Manipulations of Said Library, Medium, Surface and/or Material

At any time or any time period before, during, or after any of the steps D1, D2, D3, or D4, the molecule, molecule library, medium, surface, and/or material may be subjected to a manipulation.

In preferred embodiments, said manipulation may comprise adjusting the temperature, e.g. by heating, applying a magnetic field, applying radiation such as microwave radiation, or light such as laser light, applying a physical influence such as shaking, rocking, spinning, vortexing, sonication.

### Manipulations of Library Molecules

In preferred embodiments, a manipulation comprises removing, changing og modifying one or more library molecule(s). For example, molecules may be removed by partitioning, molecules may be changed by cleaving a linker from a molecule and molecules may be modified by reacting a fragment with a molecule.

In other preferred embodiments, a manipulation comprises diluting of changing the location of one or more library molecule(s). For example, molecules may be diluted by adding more medium or more of an entity of a medium. As another example, the location of one or more molecules may be changed by density centrifugation.

In yet other preferred embodiments, a manipulation comprises changing the composition of one or more characteristics of one or more library molecule(s). For example, the composition of a molecule may be changed by melting a double stranded nucleotide moiety of a molecule. As another example, a characteristic such as a shape of a molecule may be changed by subjecting the molecule to electromagnetic radiation.

### Manipulations of Linkers

Cleavable linkers may be cleaved, e.g. to release tags from M-L-T complexes bound to a surface/material. Said cleavage may be performed using UV light or using an enzyme as appropriate.

### Manipulations of Tags

At or during any step of the method of the present invention, tags may be manipulated. For example, DNA tags may be amplified by PCR, DNA tags may be cleaved by an enzyme, single-stranded DNA tags may be converted to double-stranded tags and vice versa.

### Manipulations of a Medium

In preferred embodiments, a manipulation comprises removing or changing or modifying the medium. For example, changing the medium may comprise a washing step.

In preferred embodiments, a manipulation comprises diluting the medium or changing the location of the medium. For example, diluting the medium may comprise adding an additional amount of one or more components of a medium.

In preferred embodiments, a manipulation comprises changing the composition or one or more characteristics of the medium. For example, a buffering entity may be added to a medium.

### Blocking Agent

At any time or during any time period before, during or after any of the mentioned steps or substeps, a blocking agent may be added to molecules, tags, medium, surface or material.

Said blocking agent may comprise skimmed milk, skimmed milk proteins, bacteria extract, mammalian cell extract, yeast extract, milk, cetyltrimethyl ammonium bromide (CTAB), hexadecyltrimethyl ammonium bromide (HTAB), an ionic molecule, such as a detergent, a non-ionic molecule, such as a nonionic detergent, a biomolecule, such as an oligonucleotide, such as a single- or double- or triple- or quadruple-stranded DNA or RNA or modified DNA or modified RNA or DNA mimic or RNA mimic, a polymer, such as a PEG chain, or one or more suitable polymer(s), such as a tag-binding polymer as described in Lamboy 2009, JACS 2009, 131(45) 16454-16460. Furthermore, a blocking agent may be a protein which is linked covalently or noncovalently to one or more molecules, tags, surfaces, or materials. Suitable proteins may be chosen from enzymes, structural proteins, and receptors and the like.

Manipulations of a Surface and/or Material

In preferred embodiments, a manipulation comprises removing or changing or modifying the surface or the material. For example, a surface may be changed by oxidation.

In preferred embodiments, a manipulation comprises diluting a surface or materials or changing the location of a surface or material. For example, the location of a material may be changed by centrifugation or by applying a magnetic field.

In preferred embodiments, a manipulation comprises changing the composition or one or more characteristics of the surface or the material. For example, the composition of a surface may be changed by covalently or noncovalently linking fragments or molecules to the surface. As another example, a characteristic of a surface may be changed by placing the surface on another surface.

Step F. Partitioning or Isolating a Fraction of Said Library

At any time or during any time period before, during or after any of the mentioned steps or substeps a partitioning may be performed. For example, one or more molecules, the medium or a material or a surface is relocated. As another example, a partitioning may be performed such that one or more library molecules change state from a binding state where they bind the surface of the provided material to a dissolved state where they are dissolved in the medium.

In preferred embodiments, the number of library molecules which are partitioned or isolated are chosen from; 2E+0 to 1E+1, 1E+1 to 1E+2, 1E+2 to 1E+3, 1E+3 to 1E+4, 1E+4 to 1E+5, 1E+5 to 1E+6, 1E+6 to 1E+7, 1E+7 to 1E+8, 1E+8 to 1E+9, 1E+9 to 1E+10, 1E+10 to 1E+11, 1E+11 to 1E+12, 1E+12 to 1E+13, 1E+13 to 1E+14, 1E+14 to 1E+15, 1E+15 to 1E+16, 1E+16 to 1E+17, 1E+17 to 1E+18, 1E+18 to 1E+19, 1E+19 to 1E+20, 1E+20 to 1E+21, 1E+21 to 1E+22, 1E+22 to 1E+23, 1E+23 to 1E+24, 1E+24 to 1E+25, 1E+25 to 1E+26, 1E+26 to 1E+27, 1E+27 to 1E+28, 1E+28 to 1E+29, 1E+29 to 1E+30, 1E+30 to 1E+31, 1E+31 to 1E+32, 1E+32 to 1E+33, 1E+33 to 1E+34, 1E+34 to 1E+35, 1E+35 to 1E+36, 1E+36 to 1E+37, 1E+37 to 1E+38, 1E+38 to 1E+39, 1E+39 to 1E+40 molecules.

In preferred embodiments, screening methods are performed in a microfluidics setup. For example, a microfluidic-based system may be used to isolate molecules according to the present invention. Said microfluidic-based system optionally comprises a purification chip. Said microfluidic-based system preferably comprises a microfluidic architecture.

Other preferred methods for screening (isolating or partitioning molecules) are described in the following:

### Sonication

In cases where the survival of a high number of bacteriophages is the most important characteristic, a low power input will be required. Thus, depending on the context, the power input measured in watt is preferably less than 10,000, such as less than 2,000, such as less than 1,000, such as less than 800, such as less than 600, such as less than 400, such as less than 200, such as less than 100, such as less than 50, such as less than 10, such as less than 5, such as less than 1, such as less than 0.1, such as less than 0.01.

In cases where an efficient washing or elution step is the most important characteristic, a high power input will be required. Thus, depending on the context, the power input measured in watt is preferably greater than 0.01, such as greater than 0.1, such as greater than 1, such as greater than 5, such as greater than 10, such as greater than 50, such as greater than 100, such as greater than 200, such as greater than 400, such as greater than 600, such as greater than 800, such as greater than 1,000, such as greater than 2,000, such as greater than 10,000.

In cases where the survival of a high number of bacteriophages is the most important characteristic, a short sonication time will be required. Thus, depending on the context, the sonication time measured in seconds is preferably less than 100,000, such as less than 10,000, such as less than 2,000, such as less than 1,000, such as less than 800, such as less than 600, such as less than 400, such as less than 200, such as less than 100, such as less than 50, such as less than 10, such as less than 5, such as less than 2.

In cases where an efficient washing or elution step is the most important characteristic, a long sonication time will be required. Thus, depending on the context, the sonication time measured in seconds is preferably greater than 1, such as greater than 5, such as greater than 10, such as greater than 50, such as greater than 100, such as greater than 200, such as greater than 400, such as greater than 600, such as greater than 800, such as greater than 1,000, such as greater than 2,000, such as greater than 10,000, such as greater than 100,000.

### Screening by Adsorption Chromatography

In a preferred embodiment, molecules are partitioned using adsorption chromatography whereby partitioning is based mainly on differences between the adsorption affinities of molecules for a surface or material.

### Affinity Capillary Electrophoresis (ACE)

In a preferred embodiment, molecules are partitioned using ACE which may be used with mass spectroscopy as detection system.

### Affinity Screening—Mass Spectroscopy

In a preferred embodiment, molecules are partitioned using affinity screening. A preferred method comprises the following steps; (i) combining a library of molecules with a material, then (ii) separating non-binders by pressure-based ultrafiltration, (iii) reequilibrating with library-free buffer and re-separating by ultrafiltration to enrich higher affinity ligands, and (iv) eluting ligands and analyzing the eluted ligands by e.g. liquid chromatography (LC)-MS. Another preferred method comprises the following steps; Using a centrifuged 96-well plate assembly of GPC columns, including a top sample loading plate with pinholes in the bottom of each well; a central filter plate pre-loaded with GPC resin (Sephadex, Pharmacia); and a bottom collection plate for the capture of effluents containing separated molecule(s).

### Affinity Selection on Immobilized Surface

In preferred embodiments, a surface is immobilized covalently or non-covalently to a solid support such as beads, the bottom of a well of a microtiter plate, a reagent tube, a chromatographic column, or any other solid support. A library of molecules are now incubated with the surface, non-bound molecules are washed off by replacing supernatant or column buffer with fresh buffer one or more times. After washing the bound molecules are released from solid support, for example by addition of reagents, specific ligands or the like that results in the elution of the molecule, or the pH is increased or decreased to release the bound molecules, or a tag of the molecule is cleaved off from the encoded molecule with a reagent, pH change or light-induced cleavage.

### Affinity Selection on Surface in Solution or Suspension,

In a preferred embodiments, one or more molecules are incubated with a surface or material in solution. Followed by any means of isolation of the molecules bound to the target, e.g. by immunoprecipitation of the surface-molecule complexes, or centrifuging the surface particles to the bottom and identifying on or more characteristics of one or more molecules. A library of molecules is incubated with target molecules (e.g. a protein). After complex formation of bi-functional molecules with target, the complex is isolated from non-complexes, for example by the addition of polyvalent antibodies against the surface and precipitation, or is precipitated by the addition of beads that bind the surface. The latter may for example be by addition of streptavidin-coated beads that bind to pre-biotinylated surfaces. The tags recovered by precipitation can now be characterised or amplified, e.g., by PCR. In preferred embodiments, affinity selection on a surface or material in solution is followed by gel retardation, chromatographic separation e.g. size exclusion chromatography, or separation by centrifugation e.g. in a CsCl2-gradient: A library of molecules are incubated with surface. After formation of molecule-surface complexes, the complex is isolated from non-complexes, for example by gel electrophoresis or size exclusion chromatography, or any other chromatographic or non-chromatographic method that separates the surface-bound molecules from non-surface bound molecules, for example based on the difference in size and/or charge.

### Affinity Selection on Surfaces.

In a preferred embodiment one or more molecules are partitioned by affinity selection on a surface or material. For example, Particles, preferably small particles, of solid material, e.g., metal particles, metal oxide particles, grinded plastic, wood, preformed carbon nanotubes, clay, glas, silica, bacterial biofilm or biofilm of other microorganism, cement, solid paint particles, laminate, stone, marble, quartz, textile, paper, skin, hair, cell membranes, industrial membranes, epiderm, or the like, is added to a solution comprising a library of molecules. After incubation, one or more washing steps are performed, to remove unbound molecules. Then, the molecules bound to the surface, or the identifiers of the molecules bound to the surface, are released as described above, and the identifiers characterised and/or amplified as described above.

### Affinity Chromatography

In a preferred embodiment, molecules are applied to a chromatographic column. Molecules which are bound to a surface of the chromatographic column are separated from unbound molecules because they travel differently in the chromatographic column. Hence, bound and unbound molecules are separated.

### Chromatography

In a preferred embodiment, molecules are combined with a surface or material and subjected to chromatography. Material-bound molecules and unbound molecules travel differently in the chromatographic column for example due to the charge, size, hydrophobicity, shape, stoke's radius, density or another characteristic of the material. Hence, bound and unbound molecules are separated.

### Dialysis

In a preferred embodiment, molecules are combined with a surface and unbound molecules are removed by dialysis. In contrast to surface-bound molecules, molecules which do not bind a surface can cross the dialysis membrane are thereby removed, thus isolating or partitioning the library molecules.

### Exclusion Chromatography

In a preferred embodiment, separation is based mainly on exclusion effects, such as differences in molecular size and/or shape or in charge. The term Size-Exclusion Chromatography may also be used when separation is based on molecular size. The terms Gel Filtration and Gel-Permeation Chromatography (GPC) are used to describe this process when the stationary phase is a swollen gel. The term Ion-Exclusion Chromutography is specifically used for the separation of ions in an aqueous phase.

### Gradient Elution

In a preferred embodiment, the composition of the mobile phase is changed continuously or stepwise during an elution process.

### Ion-Exchange Chromatography

In a preferred embodiment, separation is based mainly on differences in the ion exchange affinities of the sample components. Ion-exchange chromatography on small particle high efficiency columns and usually utilising conductometric or spectroscopic detectors is often referred to as Ion Chromatography (IC).

### Isothermal Chromatography

In a preferred embodiment, the temperature of a column is kept constant during a separation.

Kinetic Screening, e.g. Screening for On-Rate or Off-Rate

In addition to screening molecule libraries for tight-binding variants, as measured by equilibrium binding affinity, the present invention allows the sorting (isolation) of variants which are altered in either the on-rate (kon) or the off-rate (koff) of binding to surface or material. Screening for higher on-rate may be done by using short molecule-surface incubation times, such as 1 minute, 10 seconds, 1 second. In various embodiments, a slow off-rate screening process can include one or more steps, including the addition of an incubation with a competitor molecule, dilution of the mixture, or a combination of these (e.g., dilution of the mixture in the presence of a competitor molecule). Because the effect of a slow off-rate enrichment process generally depends upon the differing dissociation rates of different molecule-surface complexes, the duration of the slow off-rate enrichment process is selected so as to retain a high proportion of molecule-surface complexes having slow dissociation rates while substantially reducing the number of molecule-surface complexes having fast dissociation rates. The slow off-rate enrichment process may be used in one or more cycles of screening. When dilution and the addition of a competitor are used in combination, they may be performed simultaneously or sequentially, in any order.

### Ligand Density Gradient

In one embodiment, molecules are isolated using a separation matrix comprised of ligands coupled to the surfaces of a porous support, such as one or more porous particles, wherein the ligands provide at least one chemical gradient within the support. Preferably, the chemical gradient is a ligand density gradient. A separation matrix comprising a ligand density gradient and a porous support may be provided by solvent-controlled diffusion of at least one reagent into the porous support. In another embodiment, molecules may be isolated by affinity chromatography, hydrophobic interaction chromatography, reverse phased chromatography, anion exchange chromatography, ionic exchange chromatography, immobilized metal affinity chromatography, hydrophobic charge induction chromatography, ion exchange chromatography, cation exchange chromatography, hydroxylapatite chromatography.

### Normal-Phase Chromatography

In a preferred embodiment, an elution procedure is used in which the stationary phase is more polar than the mobile phase. This is in contrast to reversed-phase chromatography.

Isocratic screening

In a preferred embodiment, the composition of the mobile phase in a chromatographic screening remains constant during the elution process.

### Post-Column Derivatization

A preferred embodiment is a version of reaction chromatography in which the separated sample components eluting from the column are derivatized prior to entering the detector. The derivatization process is generally carried out “on-the-fly”, i.e., during transfer of the molecules from the column to the detector. Derivatization may also be carried out before the sample enters the column or the planar medium; this is pre-column (preliminary) derivatization.

### Programmed-Flow Chromatography (Flow Programming)

In a preferred embodiment, the rate of flow of the mobile phase is changed systematically during apart or the whole of the separation.

### Programmed-Pressure Chromatography (Pressure Programming)

In a preferred embodiment, the inlet pressure of the mobile phase is changed systematically during a part or whole of the separation.

### Programmed-Temperature Chromatography (Temperature Programming)

In a preferred embodiment, the temperature of the column is changed systematically during a part or the whole of the separation.

### Pyrolysis-Gas Chromatography

In a preferred embodiment, a sample is thermally decomposed to simpler fragments before entering the column.

### Reaction Chromatography

In a preferred embodiment, the identities of the sample components are intentionally changed between sample introduction and detection. The reaction can take place upstream of the column when the chemical identity of the individual components passing through the column differs from that of the original sample, or between the column and the detector when the original sample components are separated in the column but their identity is changed prior to entering the detection device.

### Reversed-Phase Chromatography

In a preferred embodiment, an elution procedure used in liquid chromatography in which the mobile phase is significantly more polar then the stationary phase, e.g., a microporous silica-based material with chemically bonded alkyl chains.

### Frontal Affinity Chromatography

In a preferred embodiment, molecules are passed through a column onto which a material has been attached. The non-binding compounds elute early, e.g. in the void volume, while molecules with affinity for said material elute at a later time depending on the affinity for the material.

### Gel Electrophoresis

In a preferred embodiment, molecules are combined with a material and subjected to gel electrophoresis. Material-bound molecules and unbound molecules travel differently in the gel for example due to the charge, size, hydrophobicity, shape, stoke's radius, density or another characteristic of the material. Hence, bound and unbound molecules are separated.

### Screening for Affinity by Size Exclusion Chromatography

A preferred embodiment comprises the following steps:
a) surface and library molecules are first combined and allowed to reach equilibrium,
b) then the mixture is loaded into a chromatography system fitted with a reusable SEC column containing a resin for rapid separation (<20 s) of surface-bound molecules from unbound molecules components.
c) Surface-molecule complexes in the SEC eluant are monitored (e.g by UV detection)
d) and an automated system directs the surface signal peak to a reverse-phase chromatography column for dissociation, desalting,
e) and elution of molecules into an ESIMS system for identification.

### Selection by Iterative Rounds of Binding and Elution.

In a preferred embodiment, molecules are incubated with e.g. immobilised surface, e.g. a surface immobilised on streptavidin beads. After washing one or more times, the bound molecules are released from solid support by a change in pH, or by addition of an excess of ligand that binds the surface (the ligand can be e.g. a small molecule, peptide, DNA aptamer or protein that is known to bind the surface). Alternatively, the molecules may be released by degradation of the surface, induced property changes in the surface or the like. The recovered molecules are now re-applied to a surface, optionally after removal or degradation of the ligand or reagent used for elution in the previous step. Again, washing is performed, and the bound molecules eluted. The process of incubation and binding, washing and elution can be repeated many times, until eventually only molecules of high affinity remains. Then the tags of the molecules are amplified and/or characterised. Surfaces may be immobilised on columns, on beads (batch selection), on the surface of a well, or surface and molecules may interact in solution, followed by immunoprecipitation partitioning by gravity of the surface (leading to partitioning of molecules bound to the surface).

In one embodiment of the present invention, a step involves the identification of one or more molecules with one or more desired characteristic. One such characteristic may be the ability to bind to a provided surface or material. Such binding molecules may be termed ligands and may be said to have an affinity for the surface.

In various embodiments, nanotubes having different properties exhibit different buoyant densities upon association with (e.g., encapsulation by) the surface active components. The encapsulated nanotube complexes are introduced into a density gradient provided by a fluid medium and centrifuged. Over the course of the ultracentrifugation, the complexes move within the density gradient to their respective isopycnic points, that is, where their respective buoyant density matches the density of a particular layer of the density gradient. Upon sufficient centrifugation (i.e., for a selected period of time and/or at a selected rotational rate at least partially sufficient to separate the carbon nanotubes along the medium gradient), the complexes settle into multiple bands of materials according to the desirable characteristic(s) and can be removed layer by layer from the density gradient to provide separation fractions that primarily contain nanotubes having the desirable characteristic. The success of a separation can be defined as having the complexes settle into distinct bands of materials at different locations in the density gradient that are visible to human eye. For example, each band of materials can differ in colors or shades of similar colors.

Generally, density gradient centrifugation uses a fluid medium with a predefined variation in its density as a function of position within a centrifuge tube or compartment (i.e., a density gradient). Fluid media useful with the present teachings are limited only by nanotube aggregation therein to an extent precluding at least partial separation. Accordingly, aqueous and non-aqueous fluids can be used in conjunction with any substance soluble or dispersible therein, over a range of concentrations, so as to provide the medium a density gradient for use in the separation techniques described herein. Such substances can be ionic or non-ionic, non-limiting examples of which include inorganic salts and alcohols, respectively. Such a medium can include a range of aqueous iodixanol concentrations and the corresponding gradient of concentration densities. As understood by those skilled in the art, aqueous iodixanol is a common, widely used non-ionic density gradient medium. However, other media can be used in methods of the present teachings, as would be understood by those skilled in the art.

More generally, any material or compound stable, soluble or dispersible in a fluid or solvent of choice can be used as a density gradient medium. A range of densities can be formed by dissolving such a material or compound in the fluid at different concentrations, and a density gradient can be formed, for instance, in a centrifuge tube or compartment. More practically, with regard to choice of medium, the nanotubes, whether or not functionalized (e.g., by means of association with one or more surface active components), also should be soluble, stable or dispersible within the fluids/solvent or resulting density gradient. Likewise, from a practical perspective, the maximum density of the gradient medium, as determined by the solubility limit of such a material or compound in the solvent or fluid of choice, should be at least as large as the buoyant density of the particular nanotubes (and/or in composition with one or more surface active components) for a particular medium. Accordingly, any aqueous or non-aqueous density gradient medium can be used provided that the nanotubes are stable; that is, do not aggregate to an extent precluding useful separation. Alternatives to iodixanol include inorganic salts (such as CsCl, Cs2SO4, KBr, etc.), polyhydric alcohols (such as sucrose, glycerol, sorbitol, etc.), polysaccharides (such as polysucrose, dextrans, etc.), other iodinated compounds in addition to iodixanol (such as diatrizoate, nycodenz, etc.), and colloidal materials (such as Percoll®). Other parameters which can be considered upon choice of a suitable density gradient medium include the diffusion coefficient and the sedimentation coefficient, both of which can determine how quickly a gradient redistributes during centrifugation. Generally, for more shallow gradients, a larger diffusion coefficient and a smaller sedimentation coefficient are desired.

### Selection by Phase Partitioning.

In a preferred embodiment, a two- or three phase system may be set up, wherein the molecules will partition out according (at least in part) to the characteristics of the encoded molecules. Therefore, the principle allows the identification of encoded molecules that have particular preference for a certain kind of solvent. Again, the tags of the isolated molecules can be amplified and/or characterised after the selection has occurred. It may be necessary to coat the surface of the molecule, to ensure that the partitioning of the molecule is significantly correlated with the characteristics of the encoded molecule.

### Selection for Induced Properties or Assemblies of Surfaces.

In a preferred embodiment, encoded molecules are sought that induce the binding of two or more surfaces. Thus, a selection protocol for encoded molecules with the potential to induce dimerization of surfaces A and B is as follows: A library of molecules are incubated with surfaces A and B. After incubation, the solution is applied to gel electrophoresis, ultracentrifugation (e.g. CsCI-centrifugation), size exclusion chromatography, or any other kind of separation that separates the surface A-surface B-molecule-complex from un-complexed surface A and B, and other undesired complexes, such as surface A-surface B-complex. Molecules from the band or fraction corresponding to the size and/or charge of the surface A-surface B-molecule-complex is recovered, and template identifiers are then amplified and/or characterised as described above. In this case, the encoded molecule would be resynthesized, and tested in a surface dimerisation assay for its effect on the dimerisation of surface A and B.

### Stepwise Elution

In a preferred embodiment, the composition of the mobile phase is changed in steps during a single chromatographic run.

### Two-Dimensional Chromatography

In a preferred embodiment, parts or all of the separated sample components are subjected to additional separation steps. This can be done e.g., by conducting a particular fraction eluting from the column into another column (system) having different separation characteristics. When combined with additional separation steps, this may be described as Multi-Dimensional Chromatography. In planar chromatography two-dimensional chromatography refers to the chromatographic process in which the components are caused to migrate first in one direction and subsequently in a direction at right angles to the first one; the two elutions are carried out with different eluents.

Step G. Optionally Amplifying or Copying the Whole or a Part of One or More Molecules and/or Tags

### Optionally Amplifying a Tag

At any time or during any time period before, during or after any of the mentioned steps or substeps, any part of any library molecule may be amplified or duplicated. For example, a part of a library molecule comprising DNA or RNA or modified DNA, or modified RNA, or a DNA mimic or an RNA mimic may be optionally amplified by PCR and optionally sequenced. Following partitioning, tags can optionally be amplified by PCR, and cloned and sequenced to reveal the structure of the molecule encoded by the tag, or alternatively, be amplified and taken through an additional round of templated synthesis. PCR amplification methods are described in detail in U.S. Pat. Nos. 4,683,192, 4,683,202, 4,800,159, and 4,965,188, and at least in PCR Technology: Principles and Applications for DNA Amplification, H. Erlich, ed., Stockton Press, New York (1989); and PCR Protocols: A Guide to Methods and Applications, Innis et al., eds., Academic Press, San Diego. Calif. (1990). The contents of all the foregoing documents are incorporated herein by reference.

Oligonucleotide tags can be amplified using PCR with primers generating two unique cut-sites. These cut-sites can be used for multimerization of the coding region by cloning into a suitable vector for sequencing. This approach will allow simultaneously sequencing of many encoding regions. Alternatively, the PCR product is directly cloned into a suitable vector using for example TA cloning. In still another approach the identity of the molecule is established by applying the PCR product to a suitable microarray. In preferred embodiments, oligonucleotide tags have a common terminal sequence which can serve as a primer for PCR, as is known in the art.

In embodiments in which PCR is to be used to amplify the oligonucleotide tags of selected bifunctional complexes, the identifier oligonucleotides preferably include PCR primer sequences. For example, a PCR primer sequence can be included in the display oligonucleotide and/or it can be included with the first tag oligonucleotide. The oligonucleotide tag can also include a capping PCR primer sequence that follows the tag sequences.

PCR primer sequences suitable for use in the libraries of the invention are known in the art; suitable primers and methods are set forth, for example, in Innis et al., eds., PCR Protocols: A Guide to Methods and Applications, San Diego: Academic Press (1990), the contents of which are incorporated herein by reference in their entirety.

As indicated above, the nucleotide sequence of the oligonucleotide tag as part of the methods of this invention, may be determined by the use of the polymerase chain reaction (PCR).

The PCR reaction can be performed by mixing the PCR primer pair, preferably a predetermined amount thereof, with the identifier oligonucleotide, preferably a predetermined amount thereof, in a PCR buffer to form a PCR reaction admixture. The admixture is thermocycled for a number of cycles, which is typically predetermined, sufficient for the formation of a PCR reaction product. A sufficient amount of product is one that can be isolated in a sufficient amount to allow for DNA sequence determination. PCR is typically carried out by thermocycling i.e. repeatedly increasing and decreasing the temperature of a PCR reaction admixture within a temperature range whose lower limit is about 30 C to about 55 C and whose upper limit is about 90 C to about 100 C. The increasing and decreasing steps can be continuous, but is preferably phasic with time periods of relative temperature stability at each of temperatures favoring polynucleotide synthesis, denaturation and hybridization. The PCR reaction can be performed using any suitable method. Generally it occurs in a buffered aqueous solution, i. e. a PCR buffer, preferably at a pH of 7-9. Preferably, a molar excess of the primer is present. A large molar excess is preferred to improve the efficiency of the process. The PCR buffer also contains the deoxyribonucleotide triphosphates (polynucleotide synthesis substrates) dATP, dCTP, dGTP and dTTP and a polymerase, typically thermostable, all in adequate amounts for primer extension (polynucleotide synthesis) reaction. The resulting solution (PCR mixture) is-heated to about 90 C-100 C for about 1 to 10 minutes, preferably from 1 to 4 minutes. After this heating period the solution is allowed to cool to 54 C, which is preferable for primer hybridization. The synthesis reaction may occur at a temperature ranging from room temperature up to a temperature above which the polymerase no longer functions efficiently. Suitable enzymes for elongating the primer sequences include, for example, E. coli DNA polymerase I, Taq DNA polymerase, Klenow fragment of E. coli DNA polymerasel, T4 DNA polymerase, other available DNA polymerases, reverse transcriptase, and other enzymes, including heat-stable enzymes, which will facilitate combination of the nucleotides in the proper manner to form the primer extension products which are complementary to each nucleic acid strand. Generally, the synthesis will be initiated at the 3′end of each primer and proceed in the 5′direction along the template strand, until synthesis terminates, producing molecules of different lengths. The newly synthesized DNA strand and its complementary strand form a double-stranded molecule which can be used in the succeeding steps of the analysis process.

Amplification may also be done by identifying each molecule and then synthesizing each molecule in a larger amount.

When the linker is a cell or a bacteriophage, amplification of tags may be done by propagating said cells or bacteriophages as is known in the art, e.g. using the following M13 Amplification Protocol (https://www.neb.com/protocols/1/01/01/m13-amplification)
1. Grow overnight culture of F′+ E. coli (e.g. ER2738).
2. Inoculate a 20 ml culture in a 250 ml Erlenmeyer flask with 200 μL overnight E. coli culture. Add 1 μL phage suspension. Shake flask at 37° C., 250 rpm for 4-5 hrs.
3. Remove cells by centrifugation at 4500 g for 10 min. Transfer supernatant to a fresh tube. Repeat centrifugation.
4. Transfer top 16 ml of supernatant to a new tube and add 4 mL of 2.5 M NaCl/20% PEG-8000 (w/v). Briefly mix. Precipitate phage for 1 hr or overnight at 4° C.
5. Pellet phage by centrifugation at 12000 g for 15 min. Decant supernatant. Resuspend pellet in 1 mL TBS. Transfer to an eppendorf tube. Spin briefly to remove any cell debris.
6. Transfer supernatant to a fresh tube. Add 200 μL of 2.5 M NaCl/20% PEG-8000. Incubate on ice for 15-60 min. Spin 12000-14000 rpm in a benchtop centrifuge for 10 min. Discard supernatant. Spin again briefly and remove remaining supernatant with pipette. Resuspend pellet in 200 μL TBS. For long-term storage at −20° C., add 200 μL sterile glycerol.
To scale up the above protocol, use multiple culture flasks. Alternatively, after incubating 20 ml culture for 2 hrs, add the entire culture to 1 L LB. Incubate the large culture for 4 hrs, then modify the protocol to remove cells and purify phage.

### Step H. Optionally Identifying One or More Characteristics of One or More Library Molecules, Linker or Tag, or Isolating One or More Library Molecules, Linker, or Tag

At any time or during any time period before, during or after any of the mentioned steps or substeps one or more characteristics of one or more molecules may be determined. For example, the mass of a molecule may be determined by mass spectrometry. As another example the RAMAN spectrum of a library of molecules may be determined. As yet another example, the nucleotide sequence of one or more tags may be determined.

### Identification of One or More Characteristics of a Molecule

At any time or during any time period before, during or after any of the mentioned steps or substeps, any part of any library molecule may be identified using for example using mass spectroscopy, nuclear magnetic resonance, RAMAN spectroscopy, chromatography, electrophoresis, such as capillary electrophoresis, flow cytometry, hybridization, sequencing, atomic force microscopy, absorption frequency measurements.

### Sequencing

In preferred embodiments, oligomeric tags, such as oligonucleotide tags are sequenced to reveal the identity or one or more characteristics of a molecule linked to said tag.

Sequencing methods of use for detecting and quantifying tags of interest according to the present invention include without limitation single molecule sequencing, sequencing by synthesis, sequencing using arrays (hybridization and/or ligation), capillary sequencers, Sanger sequencing, constant denaturant capillary electrophoresis (CDCE), cycling temperature capillary electrophoresis (CTCE), polony sequencing, pyrosequencing, shot-gun sequencing, and the like

Commercial high throughput sequencing platforms for detecting and quantifying tags of interest and such platforms are known in the art and can include without limitation Illumina's GA, Life Technologies' SOLD, Roche's 454, Pacific Biosciences single molecule sequencing platform, Oxford Nanopore, Ion Torrent, Complete Genomics, Nimblegen, Helicos Biosciences, Lingvitae, Nabsys, and Visigen Biotechnologies. PCR methods of use for detecting and quantifying tags of interest according to the present invention include without limitation digital PCR, quantitative PCR, and competitive PCR.

Several methods may be used for comparing tags. Such methods include detecting the identity of the different tags, detecting and/or quantifying the number of molecules of each tag. Comparing tags in further embodiments of the invention can include without limitation evaluating the concentration of tags and evaluating relative concentrations and/or relative numbers of molecules.

In a further embodiment, relative concentrations or numbers of molecules or tags may be determined.

Methods for detecting and quantifying tags or any other sequence of interest are known in the art Such methods include without limitation any methods that detect DNA (including without limitation genomic and cDNA) and RNA (including without limitation mRNA, microRNA, and silent RNA) Such methods for detecting nucleic acids can include without limitation sequencing methods, gel electrophoresis, mass spectrometry, detection of methylation patterns, PCR methods, high performance liquid chromatography (HPLC) and the like Methods for detecting and quantifying tags in accordance with the present invention provide the sequence of the tags present in the sample (or identify the presence of an allele of interest) and may also provide the number of molecules of each of the tags in the sample Methods for detecting and/or quantifying tags that are of use in the present invention also include methods that quantify the relative amounts, i.e., relative numbers of molecules and/or relative concentrations of two or more tags of interest in a sample.

Use of Molecules

Molecules identified according to a method of the present invention may covalently or non-covalently bind to a material, catalytically change a material, and/or induce a change of one or more characteristics of a material.

Furthermore, Molecules identified according to a method of the present invention may be used to modulate one or more characteristic(s) of a composite material. Said characteristic may be tensile strength, Young's modulus, flexibility, energy storage, conductivity, resistivity, brittleness.

For example, carbon nanotube binding peptides identified by methods of the present invention may be combined with a polymer in the synthesis of a biodegradable polymer with a high tensile strength.

Molecules identified according to a method of the present invention may be used in production of composites such as carbon nanotube or graphene composites using solution mixing, melt processing, electrospinning, in-situ polymerisation or any other method.

Molecules identified according to a method of the present invention may be used for sorting, e.g. molecules may be used for sorting different carbon nanotubes according to lengths, different chiralities or diameters. As another example, molecules may be used to sort carbon nanotubes or graphene according to presence or absence of defects or functionalizations.

Molecules identified according to a method of the present invention may be used for solubilizing or dispersion, such as solubilizing or dispersing carbon nanotubes or graphene

Molecules identified according to a method of the present invention may be used for top-down or bottom-up assembly methods.

Compositions/kits according to the present invention are preferably chosen from the following list, where CNT is carbon nanotube, GS is graphene sheet, SWNT is single-walled nanotube, MWNTs is multi-walled nanotube, and BNNT is boron nitride nanotube; a phage-encoded peptide library is a library where molecules (M) are optionally substituted peptides and the linker is a phage and the tag is phage genetic information which encodes the peptide, a polysome-encoded peptide library is a library where molecules (M) are optionally substituted peptides and the tag is RNA which encodes the peptide, a cell-encoded peptide library is a library where molecules (M) are optionally substituted peptides and the linker is a cell and the tag is cell genetic information which encodes the peptide,

A1.1.1.1: A DNA-encoded small molecule library, a medium comprising an organic solvent, a CNT, and a library molecule with an affinity for a CNT of <1E−2 M.

A1.1.1.2: A DNA-encoded small molecule library, a medium comprising an organic solvent, a CNT, and a library molecule with an affinity for a CNT of <1E−4 M.

A1.1.1.3: A DNA-encoded small molecule library, a medium comprising an organic solvent, a CNT, and a library molecule with an affinity for a CNT of <1E−6 M.

A1.1.1.4: A DNA-encoded small molecule library, a medium comprising an organic solvent, a CNT, and a library molecule with an affinity for a CNT of <1E−7 M.

A1.1.1.5: A DNA-encoded small molecule library, a medium comprising an organic solvent, a CNT, and a library molecule with an affinity for a CNT of <1E−8 M.

A1.1.1.6: A DNA-encoded small molecule library, a medium comprising an organic solvent, a CNT, and a library molecule with an affinity for a CNT of <1E−9 M.

A1.1.1.7: A DNA-encoded small molecule library, a medium comprising an organic solvent, a CNT, and a library molecule with an affinity for a CNT of <1E−10 M.

A1.1.1.8: A DNA-encoded small molecule library, a medium comprising an organic solvent, a CNT, and a library molecule with an affinity for a CNT of <1E−12 M.

A1.1.2.1: A DNA-encoded small molecule library, a medium comprising an organic solvent, a GS, and a library molecule with an affinity for a GS of <1E−2 M.

A1.1.2.2: A DNA-encoded small molecule library, a medium comprising an organic solvent, a GS, and a library molecule with an affinity for a GS of <1E−4 M.

A1.1.2.3: A DNA-encoded small molecule library, a medium comprising an organic solvent, a GS, and a library molecule with an affinity for a GS of <1E−6 M.

A1.1.2.4: A DNA-encoded small molecule library, a medium comprising an organic solvent, a GS, and a library molecule with an affinity for a GS of <1E−7 M.

A1.1.2.5: A DNA-encoded small molecule library, a medium comprising an organic solvent, a GS, and a library molecule with an affinity for a GS of <1E−8 M.

A1.1.2.6: A DNA-encoded small molecule library, a medium comprising an organic solvent, a GS, and a library molecule with an affinity for a GS of <1E−9 M.

A1.1.2.7: A DNA-encoded small molecule library, a medium comprising an organic solvent, a GS, and a library molecule with an affinity for a GS of <1E−10 M.

A1.1.2.8: A DNA-encoded small molecule library, a medium comprising an organic solvent, a GS, and a library molecule with an affinity for a GS of <1E−12 M.

A1.1.3.1: A DNA-encoded small molecule library, a medium comprising an organic solvent, a SWNT, and a library molecule with an affinity for a SWNT of <1E−2 M.

A1.1.3.2: A DNA-encoded small molecule library, a medium comprising an organic solvent, a SWNT, and a library molecule with an affinity for a SWNT of <1E−4 M.

A1.1.3.3: A DNA-encoded small molecule library, a medium comprising an organic solvent, a SWNT, and a library molecule with an affinity for a SWNT of <1E−6 M.

A1.1.3.4: A DNA-encoded small molecule library, a medium comprising an organic solvent, a SWNT, and a library molecule with an affinity for a SWNT of <1E−7 M.

A1.1.3.5: A DNA-encoded small molecule library, a medium comprising an organic solvent, a SWNT, and a library molecule with an affinity for a SWNT of <1E−8 M.

A1.1.3.6: A DNA-encoded small molecule library, a medium comprising an organic solvent, a SWNT, and a library molecule with an affinity for a SWNT of <1E−9 M.

A1.1.3.7: A DNA-encoded small molecule library, a medium comprising an organic solvent, a SWNT, and a library molecule with an affinity for a SWNT of <1E−10 M.

A1.1.3.8: A DNA-encoded small molecule library, a medium comprising an organic solvent, a SWNT, and a library molecule with an affinity for a SWNT of <1E−12 M.

A1.1.4.1: A DNA-encoded small molecule library, a medium comprising an organic solvent, a MWNT, and a library molecule with an affinity for a MWNT of <1E−2 M.

A1.1.4.2: A DNA-encoded small molecule library, a medium comprising an organic solvent, a MWNT, and a library molecule with an affinity for a MWNT of <1E−4 M.

A1.1.4.3: A DNA-encoded small molecule library, a medium comprising an organic solvent, a MWNT, and a library molecule with an affinity for a MWNT of <1E−6 M.

A1.1.4.4: A DNA-encoded small molecule library, a medium comprising an organic solvent, a MWNT, and a library molecule with an affinity for a MWNT of <1E−7 M.

A1.1.4.5: A DNA-encoded small molecule library, a medium comprising an organic solvent, a MWNT, and a library molecule with an affinity for a MWNT of <1E−8 M.

A1.1.4.6: A DNA-encoded small molecule library, a medium comprising an organic solvent, a MWNT, and a library molecule with an affinity for a MWNT of <1E−9 M.

A1.1.4.7: A DNA-encoded small molecule library, a medium comprising an organic solvent, a MWNT, and a library molecule with an affinity for a MWNT of <1E−10 M.

A1.1.4.8: A DNA-encoded small molecule library, a medium comprising an organic solvent, a MWNT, and a library molecule with an affinity for a MWNT of <1E−12 M.

A1.1.5.1: A DNA-encoded small molecule library, a medium comprising an organic solvent, a nanotube, and a library molecule with an affinity for a nanotube of <1E−2 M.

A1.1.5.2: A DNA-encoded small molecule library, a medium comprising an organic solvent, a nanotube, and a library molecule with an affinity for a nanotube of <1E−4 M.

A1.1.5.3: A DNA-encoded small molecule library, a medium comprising an organic solvent, a nanotube, and a library molecule with an affinity for a nanotube of <1E−6 M.

A1.1.5.4: A DNA-encoded small molecule library, a medium comprising an organic solvent, a nanotube, and a library molecule with an affinity for a nanotube of <1E−7 M.

A1.1.5.5: A DNA-encoded small molecule library, a medium comprising an organic solvent, a nanotube, and a library molecule with an affinity for a nanotube of <1E−8 M.

A1.1.5.6: A DNA-encoded small molecule library, a medium comprising an organic solvent, a nanotube, and a library molecule with an affinity for a nanotube of <1E−9 M.

A1.1.5.7: A DNA-encoded small molecule library, a medium comprising an organic solvent, a nanotube, and a library molecule with an affinity for a nanotube of <1E−10 M.

A1.1.5.8: A DNA-encoded small molecule library, a medium comprising an organic solvent, a nanotube, and a library molecule with an affinity for a nanotube of <1E−12 M.

A1.1.6.1: A DNA-encoded small molecule library, a medium comprising an organic solvent, a fullerene, and a library molecule with an affinity for a fullerene of <1E−2 M.

A1.1.6.2: A DNA-encoded small molecule library, a medium comprising an organic solvent, a fullerene, and a library molecule with an affinity for a fullerene of <1E−4 M.

A1.1.6.3: A DNA-encoded small molecule library, a medium comprising an organic solvent, a fullerene, and a library molecule with an affinity for a fullerene of <1E−6 M.

A1.1.6.4: A DNA-encoded small molecule library, a medium comprising an organic solvent, a fullerene, and a library molecule with an affinity for a fullerene of <1E−7 M.

A1.1.6.5: A DNA-encoded small molecule library, a medium comprising an organic solvent, a fullerene, and a library molecule with an affinity for a fullerene of <1E−8 M.

A1.1.6.6: A DNA-encoded small molecule library, a medium comprising an organic solvent, a fullerene, and a library molecule with an affinity for a fullerene of <1E−9 M.

A1.1.6.7: A DNA-encoded small molecule library, a medium comprising an organic solvent, a fullerene, and a library molecule with an affinity for a fullerene of <1E−10 M.

A1.1.6.8: A DNA-encoded small molecule library, a medium comprising an organic solvent, a fullerene, and a library molecule with an affinity for a fullerene of <1E−12 M.

A1.1.7.1: A DNA-encoded small molecule library, a medium comprising an organic solvent, a BNNT, and a library molecule with an affinity for a BNNT of <1E−2 M.

A1.1.7.2: A DNA-encoded small molecule library, a medium comprising an organic solvent, a BNNT, and a library molecule with an affinity for a BNNT of <1E−4 M.

A1.1.7.3: A DNA-encoded small molecule library, a medium comprising an organic solvent, a BNNT, and a library molecule with an affinity for a BNNT of <1E−6 M.

A1.1.7.4: A DNA-encoded small molecule library, a medium comprising an organic solvent, a BNNT, and a library molecule with an affinity for a BNNT of <1E−7 M.

A1.1.7.5: A DNA-encoded small molecule library, a medium comprising an organic solvent, a BNNT, and a library molecule with an affinity for a BNNT of <1E−8 M.

A1.1.7.6: A DNA-encoded small molecule library, a medium comprising an organic solvent, a BNNT, and a library molecule with an affinity for a BNNT of <1E−9 M.

A1.1.7.7: A DNA-encoded small molecule library, a medium comprising an organic solvent, a BNNT, and a library molecule with an affinity for a BNNT of <1E−10 M.

A1.1.7.8: A DNA-encoded small molecule library, a medium comprising an organic solvent, a BNNT, and a library molecule with an affinity for a BNNT of <1E−12 M.

A1.2.1.1: A DNA-encoded small molecule library, a medium comprising an organic solvent and a soluble polymer, a CNT, and a library molecule with an affinity for a CNT of <1E−2 M.

A1.2.1.2: A DNA-encoded small molecule library, a medium comprising an organic solvent and a soluble polymer, a CNT, and a library molecule with an affinity for a CNT of <1E−4 M.

A1.2.1.3: A DNA-encoded small molecule library, a medium comprising an organic solvent and a soluble polymer, a CNT, and a library molecule with an affinity for a CNT of <1E−6 M.

A1.2.1.4: A DNA-encoded small molecule library, a medium comprising an organic solvent and a soluble polymer, a CNT, and a library molecule with an affinity for a CNT of <1E−7 M.

A1.2.1.5: A DNA-encoded small molecule library, a medium comprising an organic solvent and a soluble polymer, a CNT, and a library molecule with an affinity for a CNT of <1E−8 M.

A1.2.1.6: A DNA-encoded small molecule library, a medium comprising an organic solvent and a soluble polymer, a CNT, and a library molecule with an affinity for a CNT of <1E−9 M.

A1.2.1.7: A DNA-encoded small molecule library, a medium comprising an organic solvent and a soluble polymer, a CNT, and a library molecule with an affinity for a CNT of <1E−10 M.

A1.2.1.8: A DNA-encoded small molecule library, a medium comprising an organic solvent and a soluble polymer, a CNT, and a library molecule with an affinity for a CNT of <1E−12 M.

A1.2.2.1: A DNA-encoded small molecule library, a medium comprising an organic solvent and a soluble polymer, a GS, and a library molecule with an affinity for a GS of <1E−2 M.

A1.2.2.2: A DNA-encoded small molecule library, a medium comprising an organic solvent and a soluble polymer, a GS, and a library molecule with an affinity for a GS of <1E−4 M.

A1.2.2.3: A DNA-encoded small molecule library, a medium comprising an organic solvent and a soluble polymer, a GS, and a library molecule with an affinity for a GS of <1E−6 M.

A1.2.2.4: A DNA-encoded small molecule library, a medium comprising an organic solvent and a soluble polymer, a GS, and a library molecule with an affinity for a GS of <1E−7 M.

A1.2.2.5: A DNA-encoded small molecule library, a medium comprising an organic solvent and a soluble polymer, a GS, and a library molecule with an affinity for a GS of <1E−8 M.

A1.2.2.6: A DNA-encoded small molecule library, a medium comprising an organic solvent and a soluble polymer, a GS, and a library molecule with an affinity for a GS of <1E−9 M.

A1.2.2.7: A DNA-encoded small molecule library, a medium comprising an organic solvent and a soluble polymer, a GS, and a library molecule with an affinity for a GS of <1E−10 M.

A1.2.2.8: A DNA-encoded small molecule library, a medium comprising an organic solvent and a soluble polymer, a GS, and a library molecule with an affinity for a GS of <1E−12 M.

A1.2.3.1: A DNA-encoded small molecule library, a medium comprising an organic solvent and a soluble polymer, a SWNT, and a library molecule with an affinity for a SWNT of <1E−2 M.

A1.2.3.2: A DNA-encoded small molecule library, a medium comprising an organic solvent and a soluble polymer, a SWNT, and a library molecule with an affinity for a SWNT of <1E−4 M.

A1.2.3.3: A DNA-encoded small molecule library, a medium comprising an organic solvent and a soluble polymer, a SWNT, and a library molecule with an affinity for a SWNT of <1E−6 M.

A1.2.3.4: A DNA-encoded small molecule library, a medium comprising an organic solvent and a soluble polymer, a SWNT, and a library molecule with an affinity for a SWNT of <1E−7 M.

A1.2.3.5: A DNA-encoded small molecule library, a medium comprising an organic solvent and a soluble polymer, a SWNT, and a library molecule with an affinity for a SWNT of <1E−8 M.

A1.2.3.6: A DNA-encoded small molecule library, a medium comprising an organic solvent and a soluble polymer, a SWNT, and a library molecule with an affinity for a SWNT of <1E−9 M.

A1.2.3.7: A DNA-encoded small molecule library, a medium comprising an organic solvent and a soluble polymer, a SWNT, and a library molecule with an affinity for a SWNT of <1E−10 M.

A1.2.3.8: A DNA-encoded small molecule library, a medium comprising an organic solvent and a soluble polymer, a SWNT, and a library molecule with an affinity for a SWNT of <1E−12 M.

A1.2.4.1: A DNA-encoded small molecule library, a medium comprising an organic solvent and a soluble polymer, a MWNT, and a library molecule with an affinity for a MWNT of <1E−2 M.

A1.2.4.2: A DNA-encoded small molecule library, a medium comprising an organic solvent and a soluble polymer, a MWNT, and a library molecule with an affinity for a MWNT of <1E−4 M.

A1.2.4.3: A DNA-encoded small molecule library, a medium comprising an organic solvent and a soluble polymer, a MWNT, and a library molecule with an affinity for a MWNT of <1E−6 M.

A1.2.4.4: A DNA-encoded small molecule library, a medium comprising an organic solvent and a soluble polymer, a MWNT, and a library molecule with an affinity for a MWNT of <1E−7 M.

A1.2.4.5: A DNA-encoded small molecule library, a medium comprising an organic solvent and a soluble polymer, a MWNT, and a library molecule with an affinity for a MWNT of <1E−8 M.

A1.2.4.6: A DNA-encoded small molecule library, a medium comprising an organic solvent and a soluble polymer, a MWNT, and a library molecule with an affinity for a MWNT of <1E−9 M.

A1.2.4.7: A DNA-encoded small molecule library, a medium comprising an organic solvent and a soluble polymer, a MWNT, and a library molecule with an affinity for a MWNT of <1E−10 M.

A1.2.4.8: A DNA-encoded small molecule library, a medium comprising an organic solvent and a soluble polymer, a MWNT, and a library molecule with an affinity for a MWNT of <1E−12 M.

A1.2.5.1: A DNA-encoded small molecule library, a medium comprising an organic solvent and a soluble polymer, a nanotube, and a library molecule with an affinity for a nanotube of <1E−2 M.

A1.2.5.2: A DNA-encoded small molecule library, a medium comprising an organic solvent and a soluble polymer, a nanotube, and a library molecule with an affinity for a nanotube of <1E−4 M.

A1.2.5.3: A DNA-encoded small molecule library, a medium comprising an organic solvent and a soluble polymer, a nanotube, and a library molecule with an affinity for a nanotube of <1E−6 M.

A1.2.5.4: A DNA-encoded small molecule library, a medium comprising an organic solvent and a soluble polymer, a nanotube, and a library molecule with an affinity for a nanotube of <1E−7 M.

A1.2.5.5: A DNA-encoded small molecule library, a medium comprising an organic solvent and a soluble polymer, a nanotube, and a library molecule with an affinity for a nanotube of <1E−8 M.

A1.2.5.6: A DNA-encoded small molecule library, a medium comprising an organic solvent and a soluble polymer, a nanotube, and a library molecule with an affinity for a nanotube of <1E−9 M.

A1.2.5.7: A DNA-encoded small molecule library, a medium comprising an organic solvent and a soluble polymer, a nanotube, and a library molecule with an affinity for a nanotube of <1E−10 M.

A1.2.5.8: A DNA-encoded small molecule library, a medium comprising an organic solvent and a soluble polymer, a nanotube, and a library molecule with an affinity for a nanotube of <1E−12 M.

A1.2.6.1: A DNA-encoded small molecule library, a medium comprising an organic solvent and a soluble polymer, a fullerene, and a library molecule with an affinity for a fullerene of <1E−2 M.

A1.2.6.2: A DNA-encoded small molecule library, a medium comprising an organic solvent and a soluble polymer, a fullerene, and a library molecule with an affinity for a fullerene of <1E−4 M.

A1.2.6.3: A DNA-encoded small molecule library, a medium comprising an organic solvent and a soluble polymer, a fullerene, and a library molecule with an affinity for a fullerene of <1E−6 M.

A1.2.6.4: A DNA-encoded small molecule library, a medium comprising an organic solvent and a soluble polymer, a fullerene, and a library molecule with an affinity for a fullerene of <1E−7 M.

A1.2.6.5: A DNA-encoded small molecule library, a medium comprising an organic solvent and a soluble polymer, a fullerene, and a library molecule with an affinity for a fullerene of <1E−8 M.

A1.2.6.6: A DNA-encoded small molecule library, a medium comprising an organic solvent and a soluble polymer, a fullerene, and a library molecule with an affinity for a fullerene of <1E−9 M.

A1.2.6.7: A DNA-encoded small molecule library, a medium comprising an organic solvent and a soluble polymer, a fullerene, and a library molecule with an affinity for a fullerene of <1E−10 M.

A1.2.6.8: A DNA-encoded small molecule library, a medium comprising an organic solvent and a soluble polymer, a fullerene, and a library molecule with an affinity for a fullerene of <1E−12 M.

A1.2.7.1: A DNA-encoded small molecule library, a medium comprising an organic solvent and a soluble polymer, a BNNT, and a library molecule with an affinity for a BNNT of <1E−2 M.

A1.2.7.2: A DNA-encoded small molecule library, a medium comprising an organic solvent and a soluble polymer, a BNNT, and a library molecule with an affinity for a BNNT of <1E−4 M.

A1.2.7.3: A DNA-encoded small molecule library, a medium comprising an organic solvent and a soluble polymer, a BNNT, and a library molecule with an affinity for a BNNT of <1E−6 M.

A1.2.7.4: A DNA-encoded small molecule library, a medium comprising an organic solvent and a soluble polymer, a BNNT, and a library molecule with an affinity for a BNNT of <1E−7 M.

A1.2.7.5: A DNA-encoded small molecule library, a medium comprising an organic solvent and a soluble polymer, a BNNT, and a library molecule with an affinity for a BNNT of <1E−8 M.

A1.2.7.6: A DNA-encoded small molecule library, a medium comprising an organic solvent and a soluble polymer, a BNNT, and a library molecule with an affinity for a BNNT of <1E−9 M.

A1.2.7.7: A DNA-encoded small molecule library, a medium comprising an organic solvent and a soluble polymer, a BNNT, and a library molecule with an affinity for a BNNT of <1E−10 M.

A1.2.7.8: A DNA-encoded small molecule library, a medium comprising an organic solvent and a soluble polymer, a BNNT, and a library molecule with an affinity for a BNNT of <1E−12 M.

A1.3.1.1: A DNA-encoded small molecule library, a medium comprising a soluble polymer, a CNT, and a library molecule with an affinity for a CNT of <1E−2 M.

A1.3.1.2: A DNA-encoded small molecule library, a medium comprising a soluble polymer, a CNT, and a library molecule with an affinity for a CNT of <1E−4 M.

A1.3.1.3: A DNA-encoded small molecule library, a medium comprising a soluble polymer, a CNT, and a library molecule with an affinity for a CNT of <1E−6 M.

A1.3.1.4: A DNA-encoded small molecule library, a medium comprising a soluble polymer, a CNT, and a library molecule with an affinity for a CNT of <1E−7 M.

A1.3.1.5: A DNA-encoded small molecule library, a medium comprising a soluble polymer, a CNT, and a library molecule with an affinity for a CNT of <1E−8 M.

A1.3.1.6: A DNA-encoded small molecule library, a medium comprising a soluble polymer, a CNT, and a library molecule with an affinity for a CNT of <1E−9 M.

A1.3.1.7: A DNA-encoded small molecule library, a medium comprising a soluble polymer, a CNT, and a library molecule with an affinity for a CNT of <1E−10 M.

A1.3.1.8: A DNA-encoded small molecule library, a medium comprising a soluble polymer, a CNT, and a library molecule with an affinity for a CNT of <1E−12 M.

A1.3.2.1: A DNA-encoded small molecule library, a medium comprising a soluble polymer, a GS, and a library molecule with an affinity for a GS of <1E−2 M.

A1.3.2.2: A DNA-encoded small molecule library, a medium comprising a soluble polymer, a GS, and a library molecule with an affinity for a GS of <1E−4 M.

A1.3.2.3: A DNA-encoded small molecule library, a medium comprising a soluble polymer, a GS, and a library molecule with an affinity for a GS of <1E−6 M.

A1.3.2.4: A DNA-encoded small molecule library, a medium comprising a soluble polymer, a GS, and a library molecule with an affinity for a GS of <1E−7 M.

A1.3.2.5: A DNA-encoded small molecule library, a medium comprising a soluble polymer, a GS, and a library molecule with an affinity for a GS of <1E−8 M.

A1.3.2.6: A DNA-encoded small molecule library, a medium comprising a soluble polymer, a GS, and a library molecule with an affinity for a GS of <1E−9 M.

A1.3.2.7: A DNA-encoded small molecule library, a medium comprising a soluble polymer, a GS, and a library molecule with an affinity for a GS of <1E−10 M.

A1.3.2.8: A DNA-encoded small molecule library, a medium comprising a soluble polymer, a GS, and a library molecule with an affinity for a GS of <1E−12 M.

A1.3.3.1: A DNA-encoded small molecule library, a medium comprising a soluble polymer, a SWNT, and a library molecule with an affinity for a SWNT of <1E−2 M.

A1.3.3.2: A DNA-encoded small molecule library, a medium comprising a soluble polymer, a SWNT, and a library molecule with an affinity for a SWNT of <1E−4 M.

A1.3.3.3: A DNA-encoded small molecule library, a medium comprising a soluble polymer, a SWNT, and a library molecule with an affinity for a SWNT of <1E−6 M.

A1.3.3.4: A DNA-encoded small molecule library, a medium comprising a soluble polymer, a SWNT, and a library molecule with an affinity for a SWNT of <1E−7 M.

A1.3.3.5: A DNA-encoded small molecule library, a medium comprising a soluble polymer, a SWNT, and a library molecule with an affinity for a SWNT of <1E−8 M.

A1.3.3.6: A DNA-encoded small molecule library, a medium comprising a soluble polymer, a SWNT, and a library molecule with an affinity for a SWNT of <1E−9 M.

A1.3.3.7: A DNA-encoded small molecule library, a medium comprising a soluble polymer, a SWNT, and a library molecule with an affinity for a SWNT of <1E−10 M.

A1.3.3.8: A DNA-encoded small molecule library, a medium comprising a soluble polymer, a SWNT, and a library molecule with an affinity for a SWNT of <1E−12 M.

A1.3.4.1: A DNA-encoded small molecule library, a medium comprising a soluble polymer, a MWNT, and a library molecule with an affinity for a MWNT of <1E−2 M.

A1.3.4.2: A DNA-encoded small molecule library, a medium comprising a soluble polymer, a MWNT, and a library molecule with an affinity for a MWNT of <1E−4 M.

A1.3.4.3: A DNA-encoded small molecule library, a medium comprising a soluble polymer, a MWNT, and a library molecule with an affinity for a MWNT of <1E−6 M.

A1.3.4.4: A DNA-encoded small molecule library, a medium comprising a soluble polymer, a MWNT, and a library molecule with an affinity for a MWNT of <1E−7 M.

A1.3.4.5: A DNA-encoded small molecule library, a medium comprising a soluble polymer, a MWNT, and a library molecule with an affinity for a MWNT of <1E−8 M.

A1.3.4.6: A DNA-encoded small molecule library, a medium comprising a soluble polymer, a MWNT, and a library molecule with an affinity for a MWNT of <1E−9 M.

A1.3.4.7: A DNA-encoded small molecule library, a medium comprising a soluble polymer, a MWNT, and a library molecule with an affinity for a MWNT of <1E−10 M.

A1.3.4.8: A DNA-encoded small molecule library, a medium comprising a soluble polymer, a MWNT, and a library molecule with an affinity for a MWNT of <1E−12 M.

A1.3.5.1: A DNA-encoded small molecule library, a medium comprising a soluble polymer, a nanotube, and a library molecule with an affinity for a nanotube of <1E−2 M.

A1.3.5.2: A DNA-encoded small molecule library, a medium comprising a soluble polymer, a nanotube, and a library molecule with an affinity for a nanotube of <1E−4 M.

A1.3.5.3: A DNA-encoded small molecule library, a medium comprising a soluble polymer, a nanotube, and a library molecule with an affinity for a nanotube of <1E−6 M.

A1.3.5.4: A DNA-encoded small molecule library, a medium comprising a soluble polymer, a nanotube, and a library molecule with an affinity for a nanotube of <1E−7 M.

A1.3.5.5: A DNA-encoded small molecule library, a medium comprising a soluble polymer, a nanotube, and a library molecule with an affinity for a nanotube of <1E−8 M.

A1.3.5.6: A DNA-encoded small molecule library, a medium comprising a soluble polymer, a nanotube, and a library molecule with an affinity for a nanotube of <1E−9 M.

A1.3.5.7: A DNA-encoded small molecule library, a medium comprising a soluble polymer, a nanotube, and a library molecule with an affinity for a nanotube of <1E−10 M.

A1.3.5.8: A DNA-encoded small molecule library, a medium comprising a soluble polymer, a nanotube, and a library molecule with an affinity for a nanotube of <1E−12 M.

A1.3.6.1: A DNA-encoded small molecule library, a medium comprising a soluble polymer, a fullerene, and a library molecule with an affinity for a fullerene of <1E−2 M.

A1.3.6.2: A DNA-encoded small molecule library, a medium comprising a soluble polymer, a fullerene, and a library molecule with an affinity for a fullerene of <1E−4 M.

A1.3.6.3: A DNA-encoded small molecule library, a medium comprising a soluble polymer, a fullerene, and a library molecule with an affinity for a fullerene of <1E−6 M.

A1.3.6.4: A DNA-encoded small molecule library, a medium comprising a soluble polymer, a fullerene, and a library molecule with an affinity for a fullerene of <1E−7 M.

A1.3.6.5: A DNA-encoded small molecule library, a medium comprising a soluble polymer, a fullerene, and a library molecule with an affinity for a fullerene of <1E−8 M.

A1.3.6.6: A DNA-encoded small molecule library, a medium comprising a soluble polymer, a fullerene, and a library molecule with an affinity for a fullerene of <1E−9 M.

A1.3.6.7: A DNA-encoded small molecule library, a medium comprising a soluble polymer, a fullerene, and a library molecule with an affinity for a fullerene of <1E−10 M.

A1.3.6.8: A DNA-encoded small molecule library, a medium comprising a soluble polymer, a fullerene, and a library molecule with an affinity for a fullerene of <1E−12 M.

A1.3.7.1: A DNA-encoded small molecule library, a medium comprising a soluble polymer, a BNNT, and a library molecule with an affinity for a BNNT of <1E−2 M.

A1.3.7.2: A DNA-encoded small molecule library, a medium comprising a soluble polymer, a BNNT, and a library molecule with an affinity for a BNNT of <1E−4 M.

A1.3.7.3: A DNA-encoded small molecule library, a medium comprising a soluble polymer, a BNNT, and a library molecule with an affinity for a BNNT of <1E−6 M.

A1.3.7.4: A DNA-encoded small molecule library, a medium comprising a soluble polymer, a BNNT, and a library molecule with an affinity for a BNNT of <1E−7 M.

A1.3.7.5: A DNA-encoded small molecule library, a medium comprising a soluble polymer, a BNNT, and a library molecule with an affinity for a BNNT of <1E−8 M.

A1.3.7.6: A DNA-encoded small molecule library, a medium comprising a soluble polymer, a BNNT, and a library molecule with an affinity for a BNNT of <1E−9 M.

A1.3.7.7: A DNA-encoded small molecule library, a medium comprising a soluble polymer, a BNNT, and a library molecule with an affinity for a BNNT of <1E−10 M.

A1.3.7.8: A DNA-encoded small molecule library, a medium comprising a soluble polymer, a BNNT, and a library molecule with an affinity for a BNNT of <1E−12 M.

A1.4.1.1: A DNA-encoded small molecule library, a medium comprising >50% organic solvent, a CNT, and a library molecule with an affinity for a CNT of <1E−2 M.

A1.4.1.2: A DNA-encoded small molecule library, a medium comprising >50% organic solvent, a CNT, and a library molecule with an affinity for a CNT of <1E−4 M.

A1.4.1.3: A DNA-encoded small molecule library, a medium comprising >50% organic solvent, a CNT, and a library molecule with an affinity for a CNT of <1E−6 M.

A1.4.1.4: A DNA-encoded small molecule library, a medium comprising >50% organic solvent, a CNT, and a library molecule with an affinity for a CNT of <1E−7 M.

A1.4.1.5: A DNA-encoded small molecule library, a medium comprising >50% organic solvent, a CNT, and a library molecule with an affinity for a CNT of <1E−8 M.

A1.4.1.6: A DNA-encoded small molecule library, a medium comprising >50% organic solvent, a CNT, and a library molecule with an affinity for a CNT of <1E−9 M.

A1.4.1.7: A DNA-encoded small molecule library, a medium comprising >50% organic solvent, a CNT, and a library molecule with an affinity for a CNT of <1E−10 M.

A1.4.1.8: A DNA-encoded small molecule library, a medium comprising >50% organic solvent, a CNT, and a library molecule with an affinity for a CNT of <1E−12 M.

A1.4.2.1: A DNA-encoded small molecule library, a medium comprising >50% organic solvent, a GS, and a library molecule with an affinity for a GS of <1E−2 M.

A1.4.2.2: A DNA-encoded small molecule library, a medium comprising >50% organic solvent, a GS, and a library molecule with an affinity for a GS of <1E−4 M.

A1.4.2.3: A DNA-encoded small molecule library, a medium comprising >50% organic solvent, a GS, and a library molecule with an affinity for a GS of <1E−6 M.

A1.4.2.4: A DNA-encoded small molecule library, a medium comprising >50% organic solvent, a GS, and a library molecule with an affinity for a GS of <1E−7 M.

A1.4.2.5: A DNA-encoded small molecule library, a medium comprising >50% organic solvent, a GS, and a library molecule with an affinity for a GS of <1E−8 M.

A1.4.2.6: A DNA-encoded small molecule library, a medium comprising >50% organic solvent, a GS, and a library molecule with an affinity for a GS of <1E−9 M.

A1.4.2.7: A DNA-encoded small molecule library, a medium comprising >50% organic solvent, a GS, and a library molecule with an affinity for a GS of <1E−10 M.

A1.4.2.8: A DNA-encoded small molecule library, a medium comprising >50% organic solvent, a GS, and a library molecule with an affinity for a GS of <1E−12 M.

A1.4.3.1: A DNA-encoded small molecule library, a medium comprising >50% organic solvent, a SWNT, and a library molecule with an affinity for a SWNT of <1E−2 M.

A1.4.3.2: A DNA-encoded small molecule library, a medium comprising >50% organic solvent, a SWNT, and a library molecule with an affinity for a SWNT of <1E−4 M.

A1.4.3.3: A DNA-encoded small molecule library, a medium comprising >50% organic solvent, a SWNT, and a library molecule with an affinity for a SWNT of <1E−6 M.

A1.4.3.4: A DNA-encoded small molecule library, a medium comprising >50% organic solvent, a SWNT, and a library molecule with an affinity for a SWNT of <1E−7 M.

A1.4.3.5: A DNA-encoded small molecule library, a medium comprising >50% organic solvent, a SWNT, and a library molecule with an affinity for a SWNT of <1E−8 M.

A1.4.3.6: A DNA-encoded small molecule library, a medium comprising >50% organic solvent, a SWNT, and a library molecule with an affinity for a SWNT of <1E−9 M.

A1.4.3.7: A DNA-encoded small molecule library, a medium comprising >50% organic solvent, a SWNT, and a library molecule with an affinity for a SWNT of <1E−10 M.

A1.4.3.8: A DNA-encoded small molecule library, a medium comprising >50% organic solvent, a SWNT, and a library molecule with an affinity for a SWNT of <1E−12 M.

A1.4.4.1: A DNA-encoded small molecule library, a medium comprising >50% organic solvent, a MWNT, and a library molecule with an affinity for a MWNT of <1E−2 M.

A1.4.4.2: A DNA-encoded small molecule library, a medium comprising >50% organic solvent, a MWNT, and a library molecule with an affinity for a MWNT of <1E−4 M.

A1.4.4.3: A DNA-encoded small molecule library, a medium comprising >50% organic solvent, a MWNT, and a library molecule with an affinity for a MWNT of <1E−6 M.

A1.4.4.4: A DNA-encoded small molecule library, a medium comprising >50% organic solvent, a MWNT, and a library molecule with an affinity for a MWNT of <1E−7 M.

A1.4.4.5: A DNA-encoded small molecule library, a medium comprising >50% organic solvent, a MWNT, and a library molecule with an affinity for a MWNT of <1E−8 M.

A1.4.4.6: A DNA-encoded small molecule library, a medium comprising >50% organic solvent, a MWNT, and a library molecule with an affinity for a MWNT of <1E−9 M.

A1.4.4.7: A DNA-encoded small molecule library, a medium comprising >50% organic solvent, a MWNT, and a library molecule with an affinity for a MWNT of <1E−10 M.

A1.4.4.8: A DNA-encoded small molecule library, a medium comprising >50% organic solvent, a MWNT, and a library molecule with an affinity for a MWNT of <1E−12 M.

A1.4.5.1: A DNA-encoded small molecule library, a medium comprising >50% organic solvent, a nanotube, and a library molecule with an affinity for a nanotube of <1E−2 M.

A1.4.5.2: A DNA-encoded small molecule library, a medium comprising >50% organic solvent, a nanotube, and a library molecule with an affinity for a nanotube of <1E−4 M.

A1.4.5.3: A DNA-encoded small molecule library, a medium comprising >50% organic solvent, a nanotube, and a library molecule with an affinity for a nanotube of <1E−6 M.

A1.4.5.4: A DNA-encoded small molecule library, a medium comprising >50% organic solvent, a nanotube, and a library molecule with an affinity for a nanotube of <1E−7 M.

A1.4.5.5: A DNA-encoded small molecule library, a medium comprising >50% organic solvent, a nanotube, and a library molecule with an affinity for a nanotube of <1E−8 M.

A1.4.5.6: A DNA-encoded small molecule library, a medium comprising >50% organic solvent, a nanotube, and a library molecule with an affinity for a nanotube of <1E−9 M.

A1.4.5.7: A DNA-encoded small molecule library, a medium comprising >50% organic solvent, a nanotube, and a library molecule with an affinity for a nanotube of <1E−10 M.

A1.4.5.8: A DNA-encoded small molecule library, a medium comprising >50% organic solvent, a nanotube, and a library molecule with an affinity for a nanotube of <1E−12 M.

A1.4.6.1: A DNA-encoded small molecule library, a medium comprising >50% organic solvent, a fullerene, and a library molecule with an affinity for a fullerene of <1E−2 M.

A1.4.6.2: A DNA-encoded small molecule library, a medium comprising >50% organic solvent, a fullerene, and a library molecule with an affinity for a fullerene of <1E−4 M.

A1.4.6.3: A DNA-encoded small molecule library, a medium comprising >50% organic solvent, a fullerene, and a library molecule with an affinity for a fullerene of <1E−6 M.

A1.4.6.4: A DNA-encoded small molecule library, a medium comprising >50% organic solvent, a fullerene, and a library molecule with an affinity for a fullerene of <1E−7 M.

A1.4.6.5: A DNA-encoded small molecule library, a medium comprising >50% organic solvent, a fullerene, and a library molecule with an affinity for a fullerene of <1E−8 M.

A1.4.6.6: A DNA-encoded small molecule library, a medium comprising >50% organic solvent, a fullerene, and a library molecule with an affinity for a fullerene of <1E−9 M.

A1.4.6.7: A DNA-encoded small molecule library, a medium comprising >50% organic solvent, a fullerene, and a library molecule with an affinity for a fullerene of <1E−10 M.

A1.4.6.8: A DNA-encoded small molecule library, a medium comprising >50% organic solvent, a fullerene, and a library molecule with an affinity for a fullerene of <1E−12 M.

A1.4.7.1: A DNA-encoded small molecule library, a medium comprising >50% organic solvent, a BNNT, and a library molecule with an affinity for a BNNT of <1E−2 M.

A1.4.7.2: A DNA-encoded small molecule library, a medium comprising >50% organic solvent, a BNNT, and a library molecule with an affinity for a BNNT of <1E−4 M.

A1.4.7.3: A DNA-encoded small molecule library, a medium comprising >50% organic solvent, a BNNT, and a library molecule with an affinity for a BNNT of <1E−6 M.

A1.4.7.4: A DNA-encoded small molecule library, a medium comprising >50% organic solvent, a BNNT, and a library molecule with an affinity for a BNNT of <1E−7 M.

A1.4.7.5: A DNA-encoded small molecule library, a medium comprising >50% organic solvent, a BNNT, and a library molecule with an affinity for a BNNT of <1E−8 M.

A1.4.7.6: A DNA-encoded small molecule library, a medium comprising >50% organic solvent, a BNNT, and a library molecule with an affinity for a BNNT of <1E−9 M.

A1.4.7.7: A DNA-encoded small molecule library, a medium comprising >50% organic solvent, a BNNT, and a library molecule with an affinity for a BNNT of <1E−10 M.

A1.4.7.8: A DNA-encoded small molecule library, a medium comprising >50% organic solvent, a BNNT, and a library molecule with an affinity for a BNNT of <1E−12 M.

A1.5.1.1: A DNA-encoded small molecule library, a medium comprising >50% organic solvent and a soluble polymer, a CNT, and a library molecule with an affinity for a CNT of <1E−2 M.

A1.5.1.2: A DNA-encoded small molecule library, a medium comprising >50% organic solvent and a soluble polymer, a CNT, and a library molecule with an affinity for a CNT of <1E−4 M.

A1.5.1.3: A DNA-encoded small molecule library, a medium comprising >50% organic solvent and a soluble polymer, a CNT, and a library molecule with an affinity for a CNT of <1E−6 M.

A1.5.1.4: A DNA-encoded small molecule library, a medium comprising >50% organic solvent and a soluble polymer, a CNT, and a library molecule with an affinity for a CNT of <1E−7 M.

A1.5.1.5: A DNA-encoded small molecule library, a medium comprising >50% organic solvent and a soluble polymer, a CNT, and a library molecule with an affinity for a CNT of <1E−8 M.

A1.5.1.6: A DNA-encoded small molecule library, a medium comprising >50% organic solvent and a soluble polymer, a CNT, and a library molecule with an affinity for a CNT of <1E−9 M.

A1.5.1.7: A DNA-encoded small molecule library, a medium comprising >50% organic solvent and a soluble polymer, a CNT, and a library molecule with an affinity for a CNT of <1E−10 M.

A1.5.1.8: A DNA-encoded small molecule library, a medium comprising >50% organic solvent and a soluble polymer, a CNT, and a library molecule with an affinity for a CNT of <1E−12 M.

A1.5.2.1: A DNA-encoded small molecule library, a medium comprising >50% organic solvent and a soluble polymer, a GS, and a library molecule with an affinity for a GS of <1E−2 M.

A1.5.2.2: A DNA-encoded small molecule library, a medium comprising >50% organic solvent and a soluble polymer, a GS, and a library molecule with an affinity for a GS of <1E−4 M.

A1.5.2.3: A DNA-encoded small molecule library, a medium comprising >50% organic solvent and a soluble polymer, a GS, and a library molecule with an affinity for a GS of <1E−6 M.

A1.5.2.4: A DNA-encoded small molecule library, a medium comprising >50% organic solvent and a soluble polymer, a GS, and a library molecule with an affinity for a GS of <1E−7 M.

A1.5.2.5: A DNA-encoded small molecule library, a medium comprising >50% organic solvent and a soluble polymer, a GS, and a library molecule with an affinity for a GS of <1E−8 M.

A1.5.2.6: A DNA-encoded small molecule library, a medium comprising >50% organic solvent and a soluble polymer, a GS, and a library molecule with an affinity for a GS of <1E−9 M.

A1.5.2.7: A DNA-encoded small molecule library, a medium comprising >50% organic solvent and a soluble polymer, a GS, and a library molecule with an affinity for a GS of <1E−10 M.

A1.5.2.8: A DNA-encoded small molecule library, a medium comprising >50% organic solvent and a soluble polymer, a GS, and a library molecule with an affinity for a GS of <1E−12 M.

A1.5.3.1: A DNA-encoded small molecule library, a medium comprising >50% organic solvent and a soluble polymer, a SWNT, and a library molecule with an affinity for a SWNT of <1E−2 M.

A1.5.3.2: A DNA-encoded small molecule library, a medium comprising >50% organic solvent and a soluble polymer, a SWNT, and a library molecule with an affinity for a SWNT of <1E−4 M.

A1.5.3.3: A DNA-encoded small molecule library, a medium comprising >50% organic solvent and a soluble polymer, a SWNT, and a library molecule with an affinity for a SWNT of <1E−6 M.

A1.5.3.4: A DNA-encoded small molecule library, a medium comprising >50% organic solvent and a soluble polymer, a SWNT, and a library molecule with an affinity for a SWNT of <1E−7 M.

A1.5.3.5: A DNA-encoded small molecule library, a medium comprising >50% organic solvent and a soluble polymer, a SWNT, and a library molecule with an affinity for a SWNT of <1E−8 M.

A1.5.3.6: A DNA-encoded small molecule library, a medium comprising >50% organic solvent and a soluble polymer, a SWNT, and a library molecule with an affinity for a SWNT of <1E−9 M.

A1.5.3.7: A DNA-encoded small molecule library, a medium comprising >50% organic solvent and a soluble polymer, a SWNT, and a library molecule with an affinity for a SWNT of <1E−10 M.

A1.5.3.8: A DNA-encoded small molecule library, a medium comprising >50% organic solvent and a soluble polymer, a SWNT, and a library molecule with an affinity for a SWNT of <1E−12 M.

A1.5.4.1: A DNA-encoded small molecule library, a medium comprising >50% organic solvent and a soluble polymer, a MWNT, and a library molecule with an affinity for a MWNT of <1E−2 M.

A1.5.4.2: A DNA-encoded small molecule library, a medium comprising >50% organic solvent and a soluble polymer, a MWNT, and a library molecule with an affinity for a MWNT of <1E−4 M.

A1.5.4.3: A DNA-encoded small molecule library, a medium comprising >50% organic solvent and a soluble polymer, a MWNT, and a library molecule with an affinity for a MWNT of <1E−6 M.

A1.5.4.4: A DNA-encoded small molecule library, a medium comprising >50% organic solvent and a soluble polymer, a MWNT, and a library molecule with an affinity for a MWNT of <1E−7 M.

A1.5.4.5: A DNA-encoded small molecule library, a medium comprising >50% organic solvent and a soluble polymer, a MWNT, and a library molecule with an affinity for a MWNT of <1E−8 M.

A1.5.4.6: A DNA-encoded small molecule library, a medium comprising >50% organic solvent and a soluble polymer, a MWNT, and a library molecule with an affinity for a MWNT of <1E−9 M.

A1.5.4.7: A DNA-encoded small molecule library, a medium comprising >50% organic solvent and a soluble polymer, a MWNT, and a library molecule with an affinity for a MWNT of <1E−10 M.

A1.5.4.8: A DNA-encoded small molecule library, a medium comprising >50% organic solvent and a soluble polymer, a MWNT, and a library molecule with an affinity for a MWNT of <1E−12 M.

A1.5.5.1: A DNA-encoded small molecule library, a medium comprising >50% organic solvent and a soluble polymer, a nanotube, and a library molecule with an affinity for a nanotube of <1E−2 M.

A1.5.5.2: A DNA-encoded small molecule library, a medium comprising >50% organic solvent and a soluble polymer, a nanotube, and a library molecule with an affinity for a nanotube of <1E−4 M.

A1.5.5.3: A DNA-encoded small molecule library, a medium comprising >50% organic solvent and a soluble polymer, a nanotube, and a library molecule with an affinity for a nanotube of <1E−6 M.

A1.5.5.4: A DNA-encoded small molecule library, a medium comprising >50% organic solvent and a soluble polymer, a nanotube, and a library molecule with an affinity for a nanotube of <1E−7 M.

A1.5.5.5: A DNA-encoded small molecule library, a medium comprising >50% organic solvent and a soluble polymer, a nanotube, and a library molecule with an affinity for a nanotube of <1E−8 M.

A1.5.5.6: A DNA-encoded small molecule library, a medium comprising >50% organic solvent and a soluble polymer, a nanotube, and a library molecule with an affinity for a nanotube of <1E−9 M.

A1.5.5.7: A DNA-encoded small molecule library, a medium comprising >50% organic solvent and a soluble polymer, a nanotube, and a library molecule with an affinity for a nanotube of <1E−10 M.

A1.5.5.8: A DNA-encoded small molecule library, a medium comprising >50% organic solvent and a soluble polymer, a nanotube, and a library molecule with an affinity for a nanotube of <1E−12 M.

A1.5.6.1: A DNA-encoded small molecule library, a medium comprising >50% organic solvent and a soluble polymer, a fullerene, and a library molecule with an affinity for a fullerene of <1E−2 M.

A1.5.6.2: A DNA-encoded small molecule library, a medium comprising >50% organic solvent and a soluble polymer, a fullerene, and a library molecule with an affinity for a fullerene of <1E−4 M.

A1.5.6.3: A DNA-encoded small molecule library, a medium comprising >50% organic solvent and a soluble polymer, a fullerene, and a library molecule with an affinity for a fullerene of <1E−6 M.

A1.5.6.4: A DNA-encoded small molecule library, a medium comprising >50% organic solvent and a soluble polymer, a fullerene, and a library molecule with an affinity for a fullerene of <1E−7 M.

A1.5.6.5: A DNA-encoded small molecule library, a medium comprising >50% organic solvent and a soluble polymer, a fullerene, and a library molecule with an affinity for a fullerene of <1E−8 M.

A1.5.6.6: A DNA-encoded small molecule library, a medium comprising >50% organic solvent and a soluble polymer, a fullerene, and a library molecule with an affinity for a fullerene of <1E−9 M.

A1.5.6.7: A DNA-encoded small molecule library, a medium comprising >50% organic solvent and a soluble polymer, a fullerene, and a library molecule with an affinity for a fullerene of <1E−10 M.

A1.5.6.8: A DNA-encoded small molecule library, a medium comprising >50% organic solvent and a soluble polymer, a fullerene, and a library molecule with an affinity for a fullerene of <1E−12 M.

A1.5.7.1: A DNA-encoded small molecule library, a medium comprising >50% organic solvent and a soluble polymer, a BNNT, and a library molecule with an affinity for a BNNT of <1E−2 M.

A1.5.7.2: A DNA-encoded small molecule library, a medium comprising >50% organic solvent and a soluble polymer, a BNNT, and a library molecule with an affinity for a BNNT of <1E−4 M.

A1.5.7.3: A DNA-encoded small molecule library, a medium comprising >50% organic solvent and a soluble polymer, a BNNT, and a library molecule with an affinity for a BNNT of <1E−6 M.

A1.5.7.4: A DNA-encoded small molecule library, a medium comprising >50% organic solvent and a soluble polymer, a BNNT, and a library molecule with an affinity for a BNNT of <1E−7 M.

A1.5.7.5: A DNA-encoded small molecule library, a medium comprising >50% organic solvent and a soluble polymer, a BNNT, and a library molecule with an affinity for a BNNT of <1E−8 M.

A1.5.7.6: A DNA-encoded small molecule library, a medium comprising >50% organic solvent and a soluble polymer, a BNNT, and a library molecule with an affinity for a BNNT of <1E−9 M.

A1.5.7.7: A DNA-encoded small molecule library, a medium comprising >50% organic solvent and a soluble polymer, a BNNT, and a library molecule with an affinity for a BNNT of <1E−10 M.

A1.5.7.8: A DNA-encoded small molecule library, a medium comprising >50% organic solvent and a soluble polymer, a BNNT, and a library molecule with an affinity for a BNNT of <1E−12 M.

A1.6.1.1: A DNA-encoded small molecule library, a medium comprising >80% organic solvent, a CNT, and a library molecule with an affinity for a CNT of <1E−2 M.

A1.6.1.2: A DNA-encoded small molecule library, a medium comprising >80% organic solvent, a CNT, and a library molecule with an affinity for a CNT of <1E−4 M.

A1.6.1.3: A DNA-encoded small molecule library, a medium comprising >80% organic solvent, a CNT, and a library molecule with an affinity for a CNT of <1E−6 M.

A1.6.1.4: A DNA-encoded small molecule library, a medium comprising >80% organic solvent, a CNT, and a library molecule with an affinity for a CNT of <1E−7 M.

A1.6.1.5: A DNA-encoded small molecule library, a medium comprising >80% organic solvent, a CNT, and a library molecule with an affinity for a CNT of <1E−8 M.

A1.6.1.6: A DNA-encoded small molecule library, a medium comprising >80% organic solvent, a CNT, and a library molecule with an affinity for a CNT of <1E−9 M.

A1.6.1.7: A DNA-encoded small molecule library, a medium comprising >80% organic solvent, a CNT, and a library molecule with an affinity for a CNT of <1E−10 M.

A1.6.1.8: A DNA-encoded small molecule library, a medium comprising >80% organic solvent, a CNT, and a library molecule with an affinity for a CNT of <1E−12 M.

A1.6.2.1: A DNA-encoded small molecule library, a medium comprising >80% organic solvent, a GS, and a library molecule with an affinity for a GS of <1E−2 M.

A1.6.2.2: A DNA-encoded small molecule library, a medium comprising >80% organic solvent, a GS, and a library molecule with an affinity for a GS of <1E−4 M.

A1.6.2.3: A DNA-encoded small molecule library, a medium comprising >80% organic solvent, a GS, and a library molecule with an affinity for a GS of <1E−6 M.

A1.6.2.4: A DNA-encoded small molecule library, a medium comprising >80% organic solvent, a GS, and a library molecule with an affinity for a GS of <1E−7 M.

A1.6.2.5: A DNA-encoded small molecule library, a medium comprising >80% organic solvent, a GS, and a library molecule with an affinity for a GS of <1E−8 M.

A1.6.2.6: A DNA-encoded small molecule library, a medium comprising >80% organic solvent, a GS, and a library molecule with an affinity for a GS of <1E−9 M.

A1.6.2.7: A DNA-encoded small molecule library, a medium comprising >80% organic solvent, a GS, and a library molecule with an affinity for a GS of <1E−10 M.

A1.6.2.8: A DNA-encoded small molecule library, a medium comprising >80% organic solvent, a GS, and a library molecule with an affinity for a GS of <1E−12 M.

A1.6.3.1: A DNA-encoded small molecule library, a medium comprising >80% organic solvent, a SWNT, and a library molecule with an affinity for a SWNT of <1E−2 M.

A1.6.3.2: A DNA-encoded small molecule library, a medium comprising >80% organic solvent, a SWNT, and a library molecule with an affinity for a SWNT of <1E−4 M.

A1.6.3.3: A DNA-encoded small molecule library, a medium comprising >80% organic solvent, a SWNT, and a library molecule with an affinity for a SWNT of <1E−6 M.

A1.6.3.4: A DNA-encoded small molecule library, a medium comprising >80% organic solvent, a SWNT, and a library molecule with an affinity for a SWNT of <1E−7 M.

A1.6.3.5: A DNA-encoded small molecule library, a medium comprising >80% organic solvent, a SWNT, and a library molecule with an affinity for a SWNT of <1E−8 M.

A1.6.3.6: A DNA-encoded small molecule library, a medium comprising >80% organic solvent, a SWNT, and a library molecule with an affinity for a SWNT of <1E−9 M.

A1.6.3.7: A DNA-encoded small molecule library, a medium comprising >80% organic solvent, a SWNT, and a library molecule with an affinity for a SWNT of <1E−10 M.

A1.6.3.8: A DNA-encoded small molecule library, a medium comprising >80% organic solvent, a SWNT, and a library molecule with an affinity for a SWNT of <1E−12 M.

A1.6.4.1: A DNA-encoded small molecule library, a medium comprising >80% organic solvent, a MWNT, and a library molecule with an affinity for a MWNT of <1E−2 M.

A1.6.4.2: A DNA-encoded small molecule library, a medium comprising >80% organic solvent, a MWNT, and a library molecule with an affinity for a MWNT of <1E−4 M.

A1.6.4.3: A DNA-encoded small molecule library, a medium comprising >80% organic solvent, a MWNT, and a library molecule with an affinity for a MWNT of <1E−6 M.

A1.6.4.4: A DNA-encoded small molecule library, a medium comprising >80% organic solvent, a MWNT, and a library molecule with an affinity for a MWNT of <1E−7 M.

A1.6.4.5: A DNA-encoded small molecule library, a medium comprising >80% organic solvent, a MWNT, and a library molecule with an affinity for a MWNT of <1E−8 M.

A1.6.4.6: A DNA-encoded small molecule library, a medium comprising >80% organic solvent, a MWNT, and a library molecule with an affinity for a MWNT of <1E−9 M.

A1.6.4.7: A DNA-encoded small molecule library, a medium comprising >80% organic solvent, a MWNT, and a library molecule with an affinity for a MWNT of <1E−10 M.

A1.6.4.8: A DNA-encoded small molecule library, a medium comprising >80% organic solvent, a MWNT, and a library molecule with an affinity for a MWNT of <1E−12 M.

A1.6.5.1: A DNA-encoded small molecule library, a medium comprising >80% organic solvent, a nanotube, and a library molecule with an affinity for a nanotube of <1E−2 M.

A1.6.5.2: A DNA-encoded small molecule library, a medium comprising >80% organic solvent, a nanotube, and a library molecule with an affinity for a nanotube of <1E−4 M.

A1.6.5.3: A DNA-encoded small molecule library, a medium comprising >80% organic solvent, a nanotube, and a library molecule with an affinity for a nanotube of <1E−6 M.

A1.6.5.4: A DNA-encoded small molecule library, a medium comprising >80% organic solvent, a nanotube, and a library molecule with an affinity for a nanotube of <1E−7 M.

A1.6.5.5: A DNA-encoded small molecule library, a medium comprising >80% organic solvent, a nanotube, and a library molecule with an affinity for a nanotube of <1E−8 M.

A1.6.5.6: A DNA-encoded small molecule library, a medium comprising >80% organic solvent, a nanotube, and a library molecule with an affinity for a nanotube of <1E−9 M.

A1.6.5.7: A DNA-encoded small molecule library, a medium comprising >80% organic solvent, a nanotube, and a library molecule with an affinity for a nanotube of <1E−10 M.

A1.6.5.8: A DNA-encoded small molecule library, a medium comprising >80% organic solvent, a nanotube, and a library molecule with an affinity for a nanotube of <1E−12 M.

A1.6.6.1: A DNA-encoded small molecule library, a medium comprising >80% organic solvent, a fullerene, and a library molecule with an affinity for a fullerene of <1E−2 M.

A1.6.6.2: A DNA-encoded small molecule library, a medium comprising >80% organic solvent, a fullerene, and a library molecule with an affinity for a fullerene of <1E−4 M.

A1.6.6.3: A DNA-encoded small molecule library, a medium comprising >80% organic solvent, a fullerene, and a library molecule with an affinity for a fullerene of <1E−6 M.

A1.6.6.4: A DNA-encoded small molecule library, a medium comprising >80% organic solvent, a fullerene, and a library molecule with an affinity for a fullerene of <1E−7 M.

A1.6.6.5: A DNA-encoded small molecule library, a medium comprising >80% organic solvent, a fullerene, and a library molecule with an affinity for a fullerene of <1E−8 M.

A1.6.6.6: A DNA-encoded small molecule library, a medium comprising >80% organic solvent, a fullerene, and a library molecule with an affinity for a fullerene of <1E−9 M.

A1.6.6.7: A DNA-encoded small molecule library, a medium comprising >80% organic solvent, a fullerene, and a library molecule with an affinity for a fullerene of <1E−10 M.

A1.6.6.8: A DNA-encoded small molecule library, a medium comprising >80% organic solvent, a fullerene, and a library molecule with an affinity for a fullerene of <1E−12 M.

A1.6.7.1: A DNA-encoded small molecule library, a medium comprising >80% organic solvent, a BNNT, and a library molecule with an affinity for a BNNT of <1E−2 M.

A1.6.7.2: A DNA-encoded small molecule library, a medium comprising >80% organic solvent, a BNNT, and a library molecule with an affinity for a BNNT of <1E−4 M.

A1.6.7.3: A DNA-encoded small molecule library, a medium comprising >80% organic solvent, a BNNT, and a library molecule with an affinity for a BNNT of <1E−6 M.

A1.6.7.4: A DNA-encoded small molecule library, a medium comprising >80% organic solvent, a BNNT, and a library molecule with an affinity for a BNNT of <1E−7 M.

A1.6.7.5: A DNA-encoded small molecule library, a medium comprising >80% organic solvent, a BNNT, and a library molecule with an affinity for a BNNT of <1E−8 M.

A1.6.7.6: A DNA-encoded small molecule library, a medium comprising >80% organic solvent, a BNNT, and a library molecule with an affinity for a BNNT of <1E−9 M.

A1.6.7.7: A DNA-encoded small molecule library, a medium comprising >80% organic solvent, a BNNT, and a library molecule with an affinity for a BNNT of <1E−10 M.

A1.6.7.8: A DNA-encoded small molecule library, a medium comprising >80% organic solvent, a BNNT, and a library molecule with an affinity for a BNNT of <1E−12 M.

A1.7.1.1: A DNA-encoded small molecule library, a medium comprising >80% organic solvent and a soluble polymer, a CNT, and a library molecule with an affinity for a CNT of <1E−2 M.

A1.7.1.2: A DNA-encoded small molecule library, a medium comprising >80% organic solvent and a soluble polymer, a CNT, and a library molecule with an affinity for a CNT of <1E−4 M.

A1.7.1.3: A DNA-encoded small molecule library, a medium comprising >80% organic solvent and a soluble polymer, a CNT, and a library molecule with an affinity for a CNT of <1E−6 M.

A1.7.1.4: A DNA-encoded small molecule library, a medium comprising >80% organic solvent and a soluble polymer, a CNT, and a library molecule with an affinity for a CNT of <1E−7 M.

A1.7.1.5: A DNA-encoded small molecule library, a medium comprising >80% organic solvent and a soluble polymer, a CNT, and a library molecule with an affinity for a CNT of <1E−8 M.

A1.7.1.6: A DNA-encoded small molecule library, a medium comprising >80% organic solvent and a soluble polymer, a CNT, and a library molecule with an affinity for a CNT of <1E−9 M.

A1.7.1.7: A DNA-encoded small molecule library, a medium comprising >80% organic solvent and a soluble polymer, a CNT, and a library molecule with an affinity for a CNT of <1E−10 M.

A1.7.1.8: A DNA-encoded small molecule library, a medium comprising >80% organic solvent and a soluble polymer, a CNT, and a library molecule with an affinity for a CNT of <1E−12 M.

A1.7.2.1: A DNA-encoded small molecule library, a medium comprising >80% organic solvent and a soluble polymer, a GS, and a library molecule with an affinity for a GS of <1E−2 M.

A1.7.2.2: A DNA-encoded small molecule library, a medium comprising >80% organic solvent and a soluble polymer, a GS, and a library molecule with an affinity for a GS of <1E−4 M.

A1.7.2.3: A DNA-encoded small molecule library, a medium comprising >80% organic solvent and a soluble polymer, a GS, and a library molecule with an affinity for a GS of <1E−6 M.

A1.7.2.4: A DNA-encoded small molecule library, a medium comprising >80% organic solvent and a soluble polymer, a GS, and a library molecule with an affinity for a GS of <1E−7 M.

A1.7.2.5: A DNA-encoded small molecule library, a medium comprising >80% organic solvent and a soluble polymer, a GS, and a library molecule with an affinity for a GS of <1E−8 M.

A1.7.2.6: A DNA-encoded small molecule library, a medium comprising >80% organic solvent and a soluble polymer, a GS, and a library molecule with an affinity for a GS of <1E−9 M.

A1.7.2.7: A DNA-encoded small molecule library, a medium comprising >80% organic solvent and a soluble polymer, a GS, and a library molecule with an affinity for a GS of <1E−10 M.

A1.7.2.8: A DNA-encoded small molecule library, a medium comprising >80% organic solvent and a soluble polymer, a GS, and a library molecule with an affinity for a GS of <1E−12 M.

A1.7.3.1: A DNA-encoded small molecule library, a medium comprising >80% organic solvent and a soluble polymer, a SWNT, and a library molecule with an affinity for a SWNT of <1E−2 M.

A1.7.3.2: A DNA-encoded small molecule library, a medium comprising >80% organic solvent and a soluble polymer, a SWNT, and a library molecule with an affinity for a SWNT of <1E−4 M.

A1.7.3.3: A DNA-encoded small molecule library, a medium comprising >80% organic solvent and a soluble polymer, a SWNT, and a library molecule with an affinity for a SWNT of <1E−6 M.

A1.7.3.4: A DNA-encoded small molecule library, a medium comprising >80% organic solvent and a soluble polymer, a SWNT, and a library molecule with an affinity for a SWNT of <1E−7 M.

A1.7.3.5: A DNA-encoded small molecule library, a medium comprising >80% organic solvent and a soluble polymer, a SWNT, and a library molecule with an affinity for a SWNT of <1E−8 M.

A1.7.3.6: A DNA-encoded small molecule library, a medium comprising >80% organic solvent and a soluble polymer, a SWNT, and a library molecule with an affinity for a SWNT of <1E−9 M.

A1.7.3.7: A DNA-encoded small molecule library, a medium comprising >80% organic solvent and a soluble polymer, a SWNT, and a library molecule with an affinity for a SWNT of <1E−10 M.

A1.7.3.8: A DNA-encoded small molecule library, a medium comprising >80% organic solvent and a soluble polymer, a SWNT, and a library molecule with an affinity for a SWNT of <1E−12 M.

A1.7.4.1: A DNA-encoded small molecule library, a medium comprising >80% organic solvent and a soluble polymer, a MWNT, and a library molecule with an affinity for a MWNT of <1E−2 M.

A1.7.4.2: A DNA-encoded small molecule library, a medium comprising >80% organic solvent and a soluble polymer, a MWNT, and a library molecule with an affinity for a MWNT of <1E−4 M.

A1.7.4.3: A DNA-encoded small molecule library, a medium comprising >80% organic solvent and a soluble polymer, a MWNT, and a library molecule with an affinity for a MWNT of <1E−6 M.

A1.7.4.4: A DNA-encoded small molecule library, a medium comprising >80% organic solvent and a soluble polymer, a MWNT, and a library molecule with an affinity for a MWNT of <1E−7 M.

A1.7.4.5: A DNA-encoded small molecule library, a medium comprising >80% organic solvent and a soluble polymer, a MWNT, and a library molecule with an affinity for a MWNT of <1E−8 M.

A1.7.4.6: A DNA-encoded small molecule library, a medium comprising >80% organic solvent and a soluble polymer, a MWNT, and a library molecule with an affinity for a MWNT of <1E−9 M.

A1.7.4.7: A DNA-encoded small molecule library, a medium comprising >80% organic solvent and a soluble polymer, a MWNT, and a library molecule with an affinity for a MWNT of <1E−10 M.

A1.7.4.8: A DNA-encoded small molecule library, a medium comprising >80% organic solvent and a soluble polymer, a MWNT, and a library molecule with an affinity for a MWNT of <1E−12 M.

A1.7.5.1: A DNA-encoded small molecule library, a medium comprising >80% organic solvent and a soluble polymer, a nanotube, and a library molecule with an affinity for a nanotube of <1E−2 M.

A1.7.5.2: A DNA-encoded small molecule library, a medium comprising >80% organic solvent and a soluble polymer, a nanotube, and a library molecule with an affinity for a nanotube of <1E−4 M.

A1.7.5.3: A DNA-encoded small molecule library, a medium comprising >80% organic solvent and a soluble polymer, a nanotube, and a library molecule with an affinity for a nanotube of <1E−6 M.

A1.7.5.4: A DNA-encoded small molecule library, a medium comprising >80% organic solvent and a soluble polymer, a nanotube, and a library molecule with an affinity for a nanotube of <1E−7 M.

A1.7.5.5: A DNA-encoded small molecule library, a medium comprising >80% organic solvent and a soluble polymer, a nanotube, and a library molecule with an affinity for a nanotube of <1E−8 M.

A1.7.5.6: A DNA-encoded small molecule library, a medium comprising >80% organic solvent and a soluble polymer, a nanotube, and a library molecule with an affinity for a nanotube of <1E−9 M.

A1.7.5.7: A DNA-encoded small molecule library, a medium comprising >80% organic solvent and a soluble polymer, a nanotube, and a library molecule with an affinity for a nanotube of <1E−10 M.

A1.7.5.8: A DNA-encoded small molecule library, a medium comprising >80% organic solvent and a soluble polymer, a nanotube, and a library molecule with an affinity for a nanotube of <1E−12 M.

A1.7.6.1: A DNA-encoded small molecule library, a medium comprising >80% organic solvent and a soluble polymer, a fullerene, and a library molecule with an affinity for a fullerene of <1E−2 M.

A1.7.6.2: A DNA-encoded small molecule library, a medium comprising >80% organic solvent and a soluble polymer, a fullerene, and a library molecule with an affinity for a fullerene of <1E−4 M.

A1.7.6.3: A DNA-encoded small molecule library, a medium comprising >80% organic solvent and a soluble polymer, a fullerene, and a library molecule with an affinity for a fullerene of <1E−6 M.

A1.7.6.4: A DNA-encoded small molecule library, a medium comprising >80% organic solvent and a soluble polymer, a fullerene, and a library molecule with an affinity for a fullerene of <1E−7 M.

A1.7.6.5: A DNA-encoded small molecule library, a medium comprising >80% organic solvent and a soluble polymer, a fullerene, and a library molecule with an affinity for a fullerene of <1E−8 M.

A1.7.6.6: A DNA-encoded small molecule library, a medium comprising >80% organic solvent and a soluble polymer, a fullerene, and a library molecule with an affinity for a fullerene of <1E−9 M.

A1.7.6.7: A DNA-encoded small molecule library, a medium comprising >80% organic solvent and a soluble polymer, a fullerene, and a library molecule with an affinity for a fullerene of <1E−10 M.

A1.7.6.8: A DNA-encoded small molecule library, a medium comprising >80% organic solvent and a soluble polymer, a fullerene, and a library molecule with an affinity for a fullerene of <1E−12 M.

A1.7.7.1: A DNA-encoded small molecule library, a medium comprising >80% organic solvent and a soluble polymer, a BNNT, and a library molecule with an affinity for a BNNT of <1E−2 M.

A1.7.7.2: A DNA-encoded small molecule library, a medium comprising >80% organic solvent and a soluble polymer, a BNNT, and a library molecule with an affinity for a BNNT of <1E−4 M.

A1.7.7.3: A DNA-encoded small molecule library, a medium comprising >80% organic solvent and a soluble polymer, a BNNT, and a library molecule with an affinity for a BNNT of <1E−6 M.

A1.7.7.4: A DNA-encoded small molecule library, a medium comprising >80% organic solvent and a soluble polymer, a BNNT, and a library molecule with an affinity for a BNNT of <1E−7 M.

A1.7.7.5: A DNA-encoded small molecule library, a medium comprising >80% organic solvent and a soluble polymer, a BNNT, and a library molecule with an affinity for a BNNT of <1E−8 M.

A1.7.7.6: A DNA-encoded small molecule library, a medium comprising >80% organic solvent and a soluble polymer, a BNNT, and a library molecule with an affinity for a BNNT of <1E−9 M.

A1.7.7.7: A DNA-encoded small molecule library, a medium comprising >80% organic solvent and a soluble polymer, a BNNT, and a library molecule with an affinity for a BNNT of <1E−10 M.

A1.7.7.8: A DNA-encoded small molecule library, a medium comprising >80% organic solvent and a soluble polymer, a BNNT, and a library molecule with an affinity for a BNNT of <1E−12 M.

A1.8.1.1: A DNA-encoded small molecule library, a medium comprising >95% organic solvent, a CNT, and a library molecule with an affinity for a CNT of <1E−2 M.

A1.8.1.2: A DNA-encoded small molecule library, a medium comprising >95% organic solvent, a CNT, and a library molecule with an affinity for a CNT of <1E−4 M.

A1.8.1.3: A DNA-encoded small molecule library, a medium comprising >95% organic solvent, a CNT, and a library molecule with an affinity for a CNT of <1E−6 M.

A1.8.1.4: A DNA-encoded small molecule library, a medium comprising >95% organic solvent, a CNT, and a library molecule with an affinity for a CNT of <1E−7 M.

A1.8.1.5: A DNA-encoded small molecule library, a medium comprising >95% organic solvent, a CNT, and a library molecule with an affinity for a CNT of <1E−8 M.

A1.8.1.6: A DNA-encoded small molecule library, a medium comprising >95% organic solvent, a CNT, and a library molecule with an affinity for a CNT of <1E−9 M.

A1.8.1.7: A DNA-encoded small molecule library, a medium comprising >95% organic solvent, a CNT, and a library molecule with an affinity for a CNT of <1E−10 M.

A1.8.1.8: A DNA-encoded small molecule library, a medium comprising >95% organic solvent, a CNT, and a library molecule with an affinity for a CNT of <1E−12 M.

A1.8.2.1: A DNA-encoded small molecule library, a medium comprising >95% organic solvent, a GS, and a library molecule with an affinity for a GS of <1E−2 M.

A1.8.2.2: A DNA-encoded small molecule library, a medium comprising >95% organic solvent, a GS, and a library molecule with an affinity for a GS of <1E−4 M.

A1.8.2.3: A DNA-encoded small molecule library, a medium comprising >95% organic solvent, a GS, and a library molecule with an affinity for a GS of <1E−6 M.

A1.8.2.4: A DNA-encoded small molecule library, a medium comprising >95% organic solvent, a GS, and a library molecule with an affinity for a GS of <1E−7 M.

A1.8.2.5: A DNA-encoded small molecule library, a medium comprising >95% organic solvent, a GS, and a library molecule with an affinity for a GS of <1E−8 M.

A1.8.2.6: A DNA-encoded small molecule library, a medium comprising >95% organic solvent, a GS, and a library molecule with an affinity for a GS of <1E−9 M.

A1.8.2.7: A DNA-encoded small molecule library, a medium comprising >95% organic solvent, a GS, and a library molecule with an affinity for a GS of <1E−10 M.

A1.8.2.8: A DNA-encoded small molecule library, a medium comprising >95% organic solvent, a GS, and a library molecule with an affinity for a GS of <1E−12 M.

A1.8.3.1: A DNA-encoded small molecule library, a medium comprising >95% organic solvent, a SWNT, and a library molecule with an affinity for a SWNT of <1E−2 M.

A1.8.3.2: A DNA-encoded small molecule library, a medium comprising >95% organic solvent, a SWNT, and a library molecule with an affinity for a SWNT of <1E−4 M.

A1.8.3.3: A DNA-encoded small molecule library, a medium comprising >95% organic solvent, a SWNT, and a library molecule with an affinity for a SWNT of <1E−6 M.

A1.8.3.4: A DNA-encoded small molecule library, a medium comprising >95% organic solvent, a SWNT, and a library molecule with an affinity for a SWNT of <1E−7 M.

A1.8.3.5: A DNA-encoded small molecule library, a medium comprising >95% organic solvent, a SWNT, and a library molecule with an affinity for a SWNT of <1E−8 M.

A1.8.3.6: A DNA-encoded small molecule library, a medium comprising >95% organic solvent, a SWNT, and a library molecule with an affinity for a SWNT of <1E−9 M.

A1.8.3.7: A DNA-encoded small molecule library, a medium comprising >95% organic solvent, a SWNT, and a library molecule with an affinity for a SWNT of <1E−10 M.

A1.8.3.8: A DNA-encoded small molecule library, a medium comprising >95% organic solvent, a SWNT, and a library molecule with an affinity for a SWNT of <1E−12 M.

A1.8.4.1: A DNA-encoded small molecule library, a medium comprising >95% organic solvent, a MWNT, and a library molecule with an affinity for a MWNT of <1E−2 M.

A1.8.4.2: A DNA-encoded small molecule library, a medium comprising >95% organic solvent, a MWNT, and a library molecule with an affinity for a MWNT of <1E−4 M.

A1.8.4.3: A DNA-encoded small molecule library, a medium comprising >95% organic solvent, a MWNT, and a library molecule with an affinity for a MWNT of <1E−6 M.

A1.8.4.4: A DNA-encoded small molecule library, a medium comprising >95% organic solvent, a MWNT, and a library molecule with an affinity for a MWNT of <1E−7 M.

A1.8.4.5: A DNA-encoded small molecule library, a medium comprising >95% organic solvent, a MWNT, and a library molecule with an affinity for a MWNT of <1E−8 M.

A1.8.4.6: A DNA-encoded small molecule library, a medium comprising >95% organic solvent, a MWNT, and a library molecule with an affinity for a MWNT of <1E−9 M.

A1.8.4.7: A DNA-encoded small molecule library, a medium comprising >95% organic solvent, a MWNT, and a library molecule with an affinity for a MWNT of <1E−10 M.

A1.8.4.8: A DNA-encoded small molecule library, a medium comprising >95% organic solvent, a MWNT, and a library molecule with an affinity for a MWNT of <1E−12 M.

A1.8.5.1: A DNA-encoded small molecule library, a medium comprising >95% organic solvent, a nanotube, and a library molecule with an affinity for a nanotube of <1E−2 M.

A1.8.5.2: A DNA-encoded small molecule library, a medium comprising >95% organic solvent, a nanotube, and a library molecule with an affinity for a nanotube of <1E−4 M.

A1.8.5.3: A DNA-encoded small molecule library, a medium comprising >95% organic solvent, a nanotube, and a library molecule with an affinity for a nanotube of <1E−6 M.

A1.8.5.4: A DNA-encoded small molecule library, a medium comprising >95% organic solvent, a nanotube, and a library molecule with an affinity for a nanotube of <1E−7 M.

A1.8.5.5: A DNA-encoded small molecule library, a medium comprising >95% organic solvent, a nanotube, and a library molecule with an affinity for a nanotube of <1E−8 M.

A1.8.5.6: A DNA-encoded small molecule library, a medium comprising >95% organic solvent, a nanotube, and a library molecule with an affinity for a nanotube of <1E−9 M.

A1.8.5.7: A DNA-encoded small molecule library, a medium comprising >95% organic solvent, a nanotube, and a library molecule with an affinity for a nanotube of <1E−10 M.

A1.8.5.8: A DNA-encoded small molecule library, a medium comprising >95% organic solvent, a nanotube, and a library molecule with an affinity for a nanotube of <1E−12 M.

A1.8.6.1: A DNA-encoded small molecule library, a medium comprising >95% organic solvent, a fullerene, and a library molecule with an affinity for a fullerene of <1E−2 M.

A1.8.6.2: A DNA-encoded small molecule library, a medium comprising >95% organic solvent, a fullerene, and a library molecule with an affinity for a fullerene of <1E−4 M.

A1.8.6.3: A DNA-encoded small molecule library, a medium comprising >95% organic solvent, a fullerene, and a library molecule with an affinity for a fullerene of <1E−6 M.

A1.8.6.4: A DNA-encoded small molecule library, a medium comprising >95% organic solvent, a fullerene, and a library molecule with an affinity for a fullerene of <1E−7 M.

A1.8.6.5: A DNA-encoded small molecule library, a medium comprising >95% organic solvent, a fullerene, and a library molecule with an affinity for a fullerene of <1E−8 M.

A1.8.6.6: A DNA-encoded small molecule library, a medium comprising >95% organic solvent, a fullerene, and a library molecule with an affinity for a fullerene of <1E−9 M.

A1.8.6.7: A DNA-encoded small molecule library, a medium comprising >95% organic solvent, a fullerene, and a library molecule with an affinity for a fullerene of <1E−10 M.

A1.8.6.8: A DNA-encoded small molecule library, a medium comprising >95% organic solvent, a fullerene, and a library molecule with an affinity for a fullerene of <1E−12 M.

A1.8.7.1: A DNA-encoded small molecule library, a medium comprising >95% organic solvent, a BNNT, and a library molecule with an affinity for a BNNT of <1E−2 M.

A1.8.7.2: A DNA-encoded small molecule library, a medium comprising >95% organic solvent, a BNNT, and a library molecule with an affinity for a BNNT of <1E−4 M.

A1.8.7.3: A DNA-encoded small molecule library, a medium comprising >95% organic solvent, a BNNT, and a library molecule with an affinity for a BNNT of <1E−6 M.

A1.8.7.4: A DNA-encoded small molecule library, a medium comprising >95% organic solvent, a BNNT, and a library molecule with an affinity for a BNNT of <1E−7 M.

A1.8.7.5: A DNA-encoded small molecule library, a medium comprising >95% organic solvent, a BNNT, and a library molecule with an affinity for a BNNT of <1E−8 M.

A1.8.7.6: A DNA-encoded small molecule library, a medium comprising >95% organic solvent, a BNNT, and a library molecule with an affinity for a BNNT of <1E−9 M.

A1.8.7.7: A DNA-encoded small molecule library, a medium comprising >95% organic solvent, a BNNT, and a library molecule with an affinity for a BNNT of <1E−10 M.

A1.8.7.8: A DNA-encoded small molecule library, a medium comprising >95% organic solvent, a BNNT, and a library molecule with an affinity for a BNNT of <1E−12 M.

A1.9.1.1: A DNA-encoded small molecule library, a medium comprising >95% organic solvent and a soluble polymer, a CNT, and a library molecule with an affinity for a CNT of <1E−2 M.

A1.9.1.2: A DNA-encoded small molecule library, a medium comprising >95% organic solvent and a soluble polymer, a CNT, and a library molecule with an affinity for a CNT of <1E−4 M.

A1.9.1.3: A DNA-encoded small molecule library, a medium comprising >95% organic solvent and a soluble polymer, a CNT, and a library molecule with an affinity for a CNT of <1E−6 M.

A1.9.1.4: A DNA-encoded small molecule library, a medium comprising >95% organic solvent and a soluble polymer, a CNT, and a library molecule with an affinity for a CNT of <1E−7 M.

A1.9.1.5: A DNA-encoded small molecule library, a medium comprising >95% organic solvent and a soluble polymer, a CNT, and a library molecule with an affinity for a CNT of <1E−8 M.

A1.9.1.6: A DNA-encoded small molecule library, a medium comprising >95% organic solvent and a soluble polymer, a CNT, and a library molecule with an affinity for a CNT of <1E−9 M.

A1.9.1.7: A DNA-encoded small molecule library, a medium comprising >95% organic solvent and a soluble polymer, a CNT, and a library molecule with an affinity for a CNT of <1E−10 M.

A1.9.1.8: A DNA-encoded small molecule library, a medium comprising >95% organic solvent and a soluble polymer, a CNT, and a library molecule with an affinity for a CNT of <1E−12 M.

A1.9.2.1: A DNA-encoded small molecule library, a medium comprising >95% organic solvent and a soluble polymer, a GS, and a library molecule with an affinity for a GS of <1E−2 M.

A1.9.2.2: A DNA-encoded small molecule library, a medium comprising >95% organic solvent and a soluble polymer, a GS, and a library molecule with an affinity for a GS of <1E−4 M.

A1.9.2.3: A DNA-encoded small molecule library, a medium comprising >95% organic solvent and a soluble polymer, a GS, and a library molecule with an affinity for a GS of <1E−6 M.

A1.9.2.4: A DNA-encoded small molecule library, a medium comprising >95% organic solvent and a soluble polymer, a GS, and a library molecule with an affinity for a GS of <1E−7 M.

A1.9.2.5: A DNA-encoded small molecule library, a medium comprising >95% organic solvent and a soluble polymer, a GS, and a library molecule with an affinity for a GS of <1E−8 M.

A1.9.2.6: A DNA-encoded small molecule library, a medium comprising >95% organic solvent and a soluble polymer, a GS, and a library molecule with an affinity for a GS of <1E−9 M.

A1.9.2.7: A DNA-encoded small molecule library, a medium comprising >95% organic solvent and a soluble polymer, a GS, and a library molecule with an affinity for a GS of <1E−10 M.

A1.9.2.8: A DNA-encoded small molecule library, a medium comprising >95% organic solvent and a soluble polymer, a GS, and a library molecule with an affinity for a GS of <1E−12 M.

A1.9.3.1: A DNA-encoded small molecule library, a medium comprising >95% organic solvent and a soluble polymer, a SWNT, and a library molecule with an affinity for a SWNT of <1E−2 M.

A1.9.3.2: A DNA-encoded small molecule library, a medium comprising >95% organic solvent and a soluble polymer, a SWNT, and a library molecule with an affinity for a SWNT of <1E−4 M.

A1.9.3.3: A DNA-encoded small molecule library, a medium comprising >95% organic solvent and a soluble polymer, a SWNT, and a library molecule with an affinity for a SWNT of <1E−6 M.

A1.9.3.4: A DNA-encoded small molecule library, a medium comprising >95% organic solvent and a soluble polymer, a SWNT, and a library molecule with an affinity for a SWNT of <1E−7 M.

A1.9.3.5: A DNA-encoded small molecule library, a medium comprising >95% organic solvent and a soluble polymer, a SWNT, and a library molecule with an affinity for a SWNT of <1E−8 M.

A1.9.3.6: A DNA-encoded small molecule library, a medium comprising >95% organic solvent and a soluble polymer, a SWNT, and a library molecule with an affinity for a SWNT of <1E−9 M.

A1.9.3.7: A DNA-encoded small molecule library, a medium comprising >95% organic solvent and a soluble polymer, a SWNT, and a library molecule with an affinity for a SWNT of <1E−10 M.

A1.9.3.8: A DNA-encoded small molecule library, a medium comprising >95% organic solvent and a soluble polymer, a SWNT, and a library molecule with an affinity for a SWNT of <1E−12 M.

A1.9.4.1: A DNA-encoded small molecule library, a medium comprising >95% organic solvent and a soluble polymer, a MWNT, and a library molecule with an affinity for a MWNT of <1E−2 M.

A1.9.4.2: A DNA-encoded small molecule library, a medium comprising >95% organic solvent and a soluble polymer, a MWNT, and a library molecule with an affinity for a MWNT of <1E−4 M.

A1.9.4.3: A DNA-encoded small molecule library, a medium comprising >95% organic solvent and a soluble polymer, a MWNT, and a library molecule with an affinity for a MWNT of <1E−6 M.

A1.9.4.4: A DNA-encoded small molecule library, a medium comprising >95% organic solvent and a soluble polymer, a MWNT, and a library molecule with an affinity for a MWNT of <1E−7 M.

A1.9.4.5: A DNA-encoded small molecule library, a medium comprising >95% organic solvent and a soluble polymer, a MWNT, and a library molecule with an affinity for a MWNT of <1E−8 M.

A1.9.4.6: A DNA-encoded small molecule library, a medium comprising >95% organic solvent and a soluble polymer, a MWNT, and a library molecule with an affinity for a MWNT of <1E−9 M.

A1.9.4.7: A DNA-encoded small molecule library, a medium comprising >95% organic solvent and a soluble polymer, a MWNT, and a library molecule with an affinity for a MWNT of <1E−10 M.

A1.9.4.8: A DNA-encoded small molecule library, a medium comprising >95% organic solvent and a soluble polymer, a MWNT, and a library molecule with an affinity for a MWNT of <1E−12 M.

A1.9.5.1: A DNA-encoded small molecule library, a medium comprising >95% organic solvent and a soluble polymer, a nanotube, and a library molecule with an affinity for a nanotube of <1E−2 M.

A1.9.5.2: A DNA-encoded small molecule library, a medium comprising >95% organic solvent and a soluble polymer, a nanotube, and a library molecule with an affinity for a nanotube of <1E−4 M.

A1.9.5.3: A DNA-encoded small molecule library, a medium comprising >95% organic solvent and a soluble polymer, a nanotube, and a library molecule with an affinity for a nanotube of <1E−6 M.

A1.9.5.4: A DNA-encoded small molecule library, a medium comprising >95% organic solvent and a soluble polymer, a nanotube, and a library molecule with an affinity for a nanotube of <1E−7 M.

A1.9.5.5: A DNA-encoded small molecule library, a medium comprising >95% organic solvent and a soluble polymer, a nanotube, and a library molecule with an affinity for a nanotube of <1E−8 M.

A1.9.5.6: A DNA-encoded small molecule library, a medium comprising >95% organic solvent and a soluble polymer, a nanotube, and a library molecule with an affinity for a nanotube of <1E−9 M.

A1.9.5.7: A DNA-encoded small molecule library, a medium comprising >95% organic solvent and a soluble polymer, a nanotube, and a library molecule with an affinity for a nanotube of <1E−10 M.

A1.9.5.8: A DNA-encoded small molecule library, a medium comprising >95% organic solvent and a soluble polymer, a nanotube, and a library molecule with an affinity for a nanotube of <1E−12 M.

A1.9.6.1: A DNA-encoded small molecule library, a medium comprising >95% organic solvent and a soluble polymer, a fullerene, and a library molecule with an affinity for a fullerene of <1E−2 M.

A1.9.6.2: A DNA-encoded small molecule library, a medium comprising >95% organic solvent and a soluble polymer, a fullerene, and a library molecule with an affinity for a fullerene of <1E−4 M.

A1.9.6.3: A DNA-encoded small molecule library, a medium comprising >95% organic solvent and a soluble polymer, a fullerene, and a library molecule with an affinity for a fullerene of <1E−6 M.

A1.9.6.4: A DNA-encoded small molecule library, a medium comprising >95% organic solvent and a soluble polymer, a fullerene, and a library molecule with an affinity for a fullerene of <1E−7 M.

A1.9.6.5: A DNA-encoded small molecule library, a medium comprising >95% organic solvent and a soluble polymer, a fullerene, and a library molecule with an affinity for a fullerene of <1E−8 M.

A1.9.6.6: A DNA-encoded small molecule library, a medium comprising >95% organic solvent and a soluble polymer, a fullerene, and a library molecule with an affinity for a fullerene of <1E−9 M.

A1.9.6.7: A DNA-encoded small molecule library, a medium comprising >95% organic solvent and a soluble polymer, a fullerene, and a library molecule with an affinity for a fullerene of <1E−10 M.

A1.9.6.8: A DNA-encoded small molecule library, a medium comprising >95% organic solvent and a soluble polymer, a fullerene, and a library molecule with an affinity for a fullerene of <1E−12 M.

A1.9.7.1: A DNA-encoded small molecule library, a medium comprising >95% organic solvent and a soluble polymer, a BNNT, and a library molecule with an affinity for a BNNT of <1E−2 M.

A1.9.7.2: A DNA-encoded small molecule library, a medium comprising >95% organic solvent and a soluble polymer, a BNNT, and a library molecule with an affinity for a BNNT of <1E−4 M.

A1.9.7.3: A DNA-encoded small molecule library, a medium comprising >95% organic solvent and a soluble polymer, a BNNT, and a library molecule with an affinity for a BNNT of <1E−6 M.

A1.9.7.4: A DNA-encoded small molecule library, a medium comprising >95% organic solvent and a soluble polymer, a BNNT, and a library molecule with an affinity for a BNNT of <1E−7 M.

A1.9.7.5: A DNA-encoded small molecule library, a medium comprising >95% organic solvent and a soluble polymer, a BNNT, and a library molecule with an affinity for a BNNT of <1E−8 M.

A1.9.7.6: A DNA-encoded small molecule library, a medium comprising >95% organic solvent and a soluble polymer, a BNNT, and a library molecule with an affinity for a BNNT of <1E−9 M.

A1.9.7.7: A DNA-encoded small molecule library, a medium comprising >95% organic solvent and a soluble polymer, a BNNT, and a library molecule with an affinity for a BNNT of <1E−10 M.

A1.9.7.8: A DNA-encoded small molecule library, a medium comprising >95% organic solvent and a soluble polymer, a BNNT, and a library molecule with an affinity for a BNNT of <1E−12 M.

A2.1.1.1: A DNA-encoded macrocycle, a medium comprising an organic solvent, a CNT, and a library molecule with an affinity for a CNT of <1E−2 M.

A2.1.1.2: A DNA-encoded macrocycle, a medium comprising an organic solvent, a CNT, and a library molecule with an affinity for a CNT of <1E−4 M.

A2.1.1.3: A DNA-encoded macrocycle, a medium comprising an organic solvent, a CNT, and a library molecule with an affinity for a CNT of <1E−6 M.

A2.1.1.4: A DNA-encoded macrocycle, a medium comprising an organic solvent, a CNT, and a library molecule with an affinity for a CNT of <1E−7 M.

A2.1.1.5: A DNA-encoded macrocycle, a medium comprising an organic solvent, a CNT, and a library molecule with an affinity for a CNT of <1E−8 M.

A2.1.1.6: A DNA-encoded macrocycle, a medium comprising an organic solvent, a CNT, and a library molecule with an affinity for a CNT of <1E−9 M.

A2.1.1.7: A DNA-encoded macrocycle, a medium comprising an organic solvent, a CNT, and a library molecule with an affinity for a CNT of <1E−10 M.

A2.1.1.8: A DNA-encoded macrocycle, a medium comprising an organic solvent, a CNT, and a library molecule with an affinity for a CNT of <1E−12 M.

A2.1.2.1: A DNA-encoded macrocycle, a medium comprising an organic solvent, a GS, and a library molecule with an affinity for a GS of <1E−2 M.

A2.1.2.2: A DNA-encoded macrocycle, a medium comprising an organic solvent, a GS, and a library molecule with an affinity for a GS of <1E−4 M.

A2.1.2.3: A DNA-encoded macrocycle, a medium comprising an organic solvent, a GS, and a library molecule with an affinity for a GS of <1E−6 M.

A2.1.2.4: A DNA-encoded macrocycle, a medium comprising an organic solvent, a GS, and a library molecule with an affinity for a GS of <1E−7 M.

A2.1.2.5: A DNA-encoded macrocycle, a medium comprising an organic solvent, a GS, and a library molecule with an affinity for a GS of <1E−8 M.

A2.1.2.6: A DNA-encoded macrocycle, a medium comprising an organic solvent, a GS, and a library molecule with an affinity for a GS of <1E−9 M.

A2.1.2.7: A DNA-encoded macrocycle, a medium comprising an organic solvent, a GS, and a library molecule with an affinity for a GS of <1E−10 M.

A2.1.2.8: A DNA-encoded macrocycle, a medium comprising an organic solvent, a GS, and a library molecule with an affinity for a GS of <1E−12 M.

A2.1.3.1: A DNA-encoded macrocycle, a medium comprising an organic solvent, a SWNT, and a library molecule with an affinity for a SWNT of <1E−2 M.

A2.1.3.2: A DNA-encoded macrocycle, a medium comprising an organic solvent, a SWNT, and a library molecule with an affinity for a SWNT of <1E−4 M.

A2.1.3.3: A DNA-encoded macrocycle, a medium comprising an organic solvent, a SWNT, and a library molecule with an affinity for a SWNT of <1E−6 M.

A2.1.3.4: A DNA-encoded macrocycle, a medium comprising an organic solvent, a SWNT, and a library molecule with an affinity for a SWNT of <1E−7 M.

A2.1.3.5: A DNA-encoded macrocycle, a medium comprising an organic solvent, a SWNT, and a library molecule with an affinity for a SWNT of <1E−8 M.

A2.1.3.6: A DNA-encoded macrocycle, a medium comprising an organic solvent, a SWNT, and a library molecule with an affinity for a SWNT of <1E−9 M.

A2.1.3.7: A DNA-encoded macrocycle, a medium comprising an organic solvent, a SWNT, and a library molecule with an affinity for a SWNT of <1E−10 M.

A2.1.3.8: A DNA-encoded macrocycle, a medium comprising an organic solvent, a SWNT, and a library molecule with an affinity for a SWNT of <1E−12 M.

A2.1.4.1: A DNA-encoded macrocycle, a medium comprising an organic solvent, a MWNT, and a library molecule with an affinity for a MWNT of <1E−2 M.

A2.1.4.2: A DNA-encoded macrocycle, a medium comprising an organic solvent, a MWNT, and a library molecule with an affinity for a MWNT of <1E−4 M.

A2.1.4.3: A DNA-encoded macrocycle, a medium comprising an organic solvent, a MWNT, and a library molecule with an affinity for a MWNT of <1E−6 M.

A2.1.4.4: A DNA-encoded macrocycle, a medium comprising an organic solvent, a MWNT, and a library molecule with an affinity for a MWNT of <1E−7 M.

A2.1.4.5: A DNA-encoded macrocycle, a medium comprising an organic solvent, a MWNT, and a library molecule with an affinity for a MWNT of <1E−8 M.

A2.1.4.6: A DNA-encoded macrocycle, a medium comprising an organic solvent, a MWNT, and a library molecule with an affinity for a MWNT of <1E−9 M.

A2.1.4.7: A DNA-encoded macrocycle, a medium comprising an organic solvent, a MWNT, and a library molecule with an affinity for a MWNT of <1E−10 M.

A2.1.4.8: A DNA-encoded macrocycle, a medium comprising an organic solvent, a MWNT, and a library molecule with an affinity for a MWNT of <1E−12 M.

A2.1.5.1: A DNA-encoded macrocycle, a medium comprising an organic solvent, a nanotube, and a library molecule with an affinity for a nanotube of <1E−2 M.

A2.1.5.2: A DNA-encoded macrocycle, a medium comprising an organic solvent, a nanotube, and a library molecule with an affinity for a nanotube of <1E−4 M.

A2.1.5.3: A DNA-encoded macrocycle, a medium comprising an organic solvent, a nanotube, and a library molecule with an affinity for a nanotube of <1E−6 M.

A2.1.5.4: A DNA-encoded macrocycle, a medium comprising an organic solvent, a nanotube, and a library molecule with an affinity for a nanotube of <1E−7 M.

A2.1.5.5: A DNA-encoded macrocycle, a medium comprising an organic solvent, a nanotube, and a library molecule with an affinity for a nanotube of <1E−8 M.

A2.1.5.6: A DNA-encoded macrocycle, a medium comprising an organic solvent, a nanotube, and a library molecule with an affinity for a nanotube of <1E−9 M.

A2.1.5.7: A DNA-encoded macrocycle, a medium comprising an organic solvent, a nanotube, and a library molecule with an affinity for a nanotube of <1E−10 M.

A2.1.5.8: A DNA-encoded macrocycle, a medium comprising an organic solvent, a nanotube, and a library molecule with an affinity for a nanotube of <1E−12 M.

A2.1.6.1: A DNA-encoded macrocycle, a medium comprising an organic solvent, a fullerene, and a library molecule with an affinity for a fullerene of <1E−2 M.

A2.1.6.2: A DNA-encoded macrocycle, a medium comprising an organic solvent, a fullerene, and a library molecule with an affinity for a fullerene of <1E−4 M.

A2.1.6.3: A DNA-encoded macrocycle, a medium comprising an organic solvent, a fullerene, and a library molecule with an affinity for a fullerene of <1E−6 M.

A2.1.6.4: A DNA-encoded macrocycle, a medium comprising an organic solvent, a fullerene, and a library molecule with an affinity for a fullerene of <1E−7 M.

A2.1.6.5: A DNA-encoded macrocycle, a medium comprising an organic solvent, a fullerene, and a library molecule with an affinity for a fullerene of <1E−8 M.

A2.1.6.6: A DNA-encoded macrocycle, a medium comprising an organic solvent, a fullerene, and a library molecule with an affinity for a fullerene of <1E−9 M.

A2.1.6.7: A DNA-encoded macrocycle, a medium comprising an organic solvent, a fullerene, and a library molecule with an affinity for a fullerene of <1E−10 M.

A2.1.6.8: A DNA-encoded macrocycle, a medium comprising an organic solvent, a fullerene, and a library molecule with an affinity for a fullerene of <1E−12 M.

A2.1.7.1: A DNA-encoded macrocycle, a medium comprising an organic solvent, a BNNT, and a library molecule with an affinity for a BNNT of <1E−2 M.

A2.1.7.2: A DNA-encoded macrocycle, a medium comprising an organic solvent, a BNNT, and a library molecule with an affinity for a BNNT of <1E−4 M.

A2.1.7.3: A DNA-encoded macrocycle, a medium comprising an organic solvent, a BNNT, and a library molecule with an affinity for a BNNT of <1E−6 M.

A2.1.7.4: A DNA-encoded macrocycle, a medium comprising an organic solvent, a BNNT, and a library molecule with an affinity for a BNNT of <1E−7 M.

A2.1.7.5: A DNA-encoded macrocycle, a medium comprising an organic solvent, a BNNT, and a library molecule with an affinity for a BNNT of <1E−8 M.

A2.1.7.6: A DNA-encoded macrocycle, a medium comprising an organic solvent, a BNNT, and a library molecule with an affinity for a BNNT of <1E−9 M.

A2.1.7.7: A DNA-encoded macrocycle, a medium comprising an organic solvent, a BNNT, and a library molecule with an affinity for a BNNT of <1E−10 M.

A2.1.7.8: A DNA-encoded macrocycle, a medium comprising an organic solvent, a BNNT, and a library molecule with an affinity for a BNNT of <1E−12 M.

A2.2.1.1: A DNA-encoded macrocycle, a medium comprising an organic solvent and a soluble polymer, a CNT, and a library molecule with an affinity for a CNT of <1E−2 M.

A2.2.1.2: A DNA-encoded macrocycle, a medium comprising an organic solvent and a soluble polymer, a CNT, and a library molecule with an affinity for a CNT of <1E−4 M.

A2.2.1.3: A DNA-encoded macrocycle, a medium comprising an organic solvent and a soluble polymer, a CNT, and a library molecule with an affinity for a CNT of <1E−6 M.

A2.2.1.4: A DNA-encoded macrocycle, a medium comprising an organic solvent and a soluble polymer, a CNT, and a library molecule with an affinity for a CNT of <1E−7 M.

A2.2.1.5: A DNA-encoded macrocycle, a medium comprising an organic solvent and a soluble polymer, a CNT, and a library molecule with an affinity for a CNT of <1E−8 M.

A2.2.1.6: A DNA-encoded macrocycle, a medium comprising an organic solvent and a soluble polymer, a CNT, and a library molecule with an affinity for a CNT of <1E−9 M.

A2.2.1.7: A DNA-encoded macrocycle, a medium comprising an organic solvent and a soluble polymer, a CNT, and a library molecule with an affinity for a CNT of <1E−10 M.

A2.2.1.8: A DNA-encoded macrocycle, a medium comprising an organic solvent and a soluble polymer, a CNT, and a library molecule with an affinity for a CNT of <1E−12 M.

A2.2.2.1: A DNA-encoded macrocycle, a medium comprising an organic solvent and a soluble polymer, a GS, and a library molecule with an affinity for a GS of <1E−2 M.

A2.2.2.2: A DNA-encoded macrocycle, a medium comprising an organic solvent and a soluble polymer, a GS, and a library molecule with an affinity for a GS of <1E−4 M.

A2.2.2.3: A DNA-encoded macrocycle, a medium comprising an organic solvent and a soluble polymer, a GS, and a library molecule with an affinity for a GS of <1E−6 M.

A2.2.2.4: A DNA-encoded macrocycle, a medium comprising an organic solvent and a soluble polymer, a GS, and a library molecule with an affinity for a GS of <1E−7 M.

A2.2.2.5: A DNA-encoded macrocycle, a medium comprising an organic solvent and a soluble polymer, a GS, and a library molecule with an affinity for a GS of <1E−8 M.

A2.2.2.6: A DNA-encoded macrocycle, a medium comprising an organic solvent and a soluble polymer, a GS, and a library molecule with an affinity for a GS of <1E−9 M.

A2.2.2.7: A DNA-encoded macrocycle, a medium comprising an organic solvent and a soluble polymer, a GS, and a library molecule with an affinity for a GS of <1E−10 M.

A2.2.2.8: A DNA-encoded macrocycle, a medium comprising an organic solvent and a soluble polymer, a GS, and a library molecule with an affinity for a GS of <1E−12 M.

A2.2.3.1: A DNA-encoded macrocycle, a medium comprising an organic solvent and a soluble polymer, a SWNT, and a library molecule with an affinity for a SWNT of <1E−2 M.

A2.2.3.2: A DNA-encoded macrocycle, a medium comprising an organic solvent and a soluble polymer, a SWNT, and a library molecule with an affinity for a SWNT of <1E−4 M.

A2.2.3.3: A DNA-encoded macrocycle, a medium comprising an organic solvent and a soluble polymer, a SWNT, and a library molecule with an affinity for a SWNT of <1E−6 M.

A2.2.3.4: A DNA-encoded macrocycle, a medium comprising an organic solvent and a soluble polymer, a SWNT, and a library molecule with an affinity for a SWNT of <1E−7 M.

A2.2.3.5: A DNA-encoded macrocycle, a medium comprising an organic solvent and a soluble polymer, a SWNT, and a library molecule with an affinity for a SWNT of <1E−8 M.

A2.2.3.6: A DNA-encoded macrocycle, a medium comprising an organic solvent and a soluble polymer, a SWNT, and a library molecule with an affinity for a SWNT of <1E−9 M.

A2.2.3.7: A DNA-encoded macrocycle, a medium comprising an organic solvent and a soluble polymer, a SWNT, and a library molecule with an affinity for a SWNT of <1E−10 M.

A2.2.3.8: A DNA-encoded macrocycle, a medium comprising an organic solvent and a soluble polymer, a SWNT, and a library molecule with an affinity for a SWNT of <1E−12 M.

A2.2.4.1: A DNA-encoded macrocycle, a medium comprising an organic solvent and a soluble polymer, a MWNT, and a library molecule with an affinity for a MWNT of <1E−2 M.

A2.2.4.2: A DNA-encoded macrocycle, a medium comprising an organic solvent and a soluble polymer, a MWNT, and a library molecule with an affinity for a MWNT of <1E−4 M.

A2.2.4.3: A DNA-encoded macrocycle, a medium comprising an organic solvent and a soluble polymer, a MWNT, and a library molecule with an affinity for a MWNT of <1E−6 M.

A2.2.4.4: A DNA-encoded macrocycle, a medium comprising an organic solvent and a soluble polymer, a MWNT, and a library molecule with an affinity for a MWNT of <1E−7 M.

A2.2.4.5: A DNA-encoded macrocycle, a medium comprising an organic solvent and a soluble polymer, a MWNT, and a library molecule with an affinity for a MWNT of <1E−8 M.

A2.2.4.6: A DNA-encoded macrocycle, a medium comprising an organic solvent and a soluble polymer, a MWNT, and a library molecule with an affinity for a MWNT of <1E−9 M.

A2.2.4.7: A DNA-encoded macrocycle, a medium comprising an organic solvent and a soluble polymer, a MWNT, and a library molecule with an affinity for a MWNT of <1E−10 M.

A2.2.4.8: A DNA-encoded macrocycle, a medium comprising an organic solvent and a soluble polymer, a MWNT, and a library molecule with an affinity for a MWNT of <1E−12 M.

A2.2.5.1: A DNA-encoded macrocycle, a medium comprising an organic solvent and a soluble polymer, a nanotube, and a library molecule with an affinity for a nanotube of <1E−2 M.

A2.2.5.2: A DNA-encoded macrocycle, a medium comprising an organic solvent and a soluble polymer, a nanotube, and a library molecule with an affinity for a nanotube of <1E−4 M.

A2.2.5.3: A DNA-encoded macrocycle, a medium comprising an organic solvent and a soluble polymer, a nanotube, and a library molecule with an affinity for a nanotube of <1E−6 M.

A2.2.5.4: A DNA-encoded macrocycle, a medium comprising an organic solvent and a soluble polymer, a nanotube, and a library molecule with an affinity for a nanotube of <1E−7 M.

A2.2.5.5: A DNA-encoded macrocycle, a medium comprising an organic solvent and a soluble polymer, a nanotube, and a library molecule with an affinity for a nanotube of <1E−8 M.

A2.2.5.6: A DNA-encoded macrocycle, a medium comprising an organic solvent and a soluble polymer, a nanotube, and a library molecule with an affinity for a nanotube of <1E−9 M.

A2.2.5.7: A DNA-encoded macrocycle, a medium comprising an organic solvent and a soluble polymer, a nanotube, and a library molecule with an affinity for a nanotube of <1E−10 M.

A2.2.5.8: A DNA-encoded macrocycle, a medium comprising an organic solvent and a soluble polymer, a nanotube, and a library molecule with an affinity for a nanotube of <1E−12 M.

A2.2.6.1: A DNA-encoded macrocycle, a medium comprising an organic solvent and a soluble polymer, a fullerene, and a library molecule with an affinity for a fullerene of <1E−2 M.

A2.2.6.2: A DNA-encoded macrocycle, a medium comprising an organic solvent and a soluble polymer, a fullerene, and a library molecule with an affinity for a fullerene of <1E−4 M.

A2.2.6.3: A DNA-encoded macrocycle, a medium comprising an organic solvent and a soluble polymer, a fullerene, and a library molecule with an affinity for a fullerene of <1E−6 M.

A2.2.6.4: A DNA-encoded macrocycle, a medium comprising an organic solvent and a soluble polymer, a fullerene, and a library molecule with an affinity for a fullerene of <1E−7 M.

A2.2.6.5: A DNA-encoded macrocycle, a medium comprising an organic solvent and a soluble polymer, a fullerene, and a library molecule with an affinity for a fullerene of <1E−8 M.

A2.2.6.6: A DNA-encoded macrocycle, a medium comprising an organic solvent and a soluble polymer, a fullerene, and a library molecule with an affinity for a fullerene of <1E−9 M.

A2.2.6.7: A DNA-encoded macrocycle, a medium comprising an organic solvent and a soluble polymer, a fullerene, and a library molecule with an affinity for a fullerene of <1E−10 M.

A2.2.6.8: A DNA-encoded macrocycle, a medium comprising an organic solvent and a soluble polymer, a fullerene, and a library molecule with an affinity for a fullerene of <1E−12 M.

A2.2.7.1: A DNA-encoded macrocycle, a medium comprising an organic solvent and a soluble polymer, a BNNT, and a library molecule with an affinity for a BNNT of <1E−2 M.

A2.2.7.2: A DNA-encoded macrocycle, a medium comprising an organic solvent and a soluble polymer, a BNNT, and a library molecule with an affinity for a BNNT of <1E−4 M.

A2.2.7.3: A DNA-encoded macrocycle, a medium comprising an organic solvent and a soluble polymer, a BNNT, and a library molecule with an affinity for a BNNT of <1E−6 M.

A2.2.7.4: A DNA-encoded macrocycle, a medium comprising an organic solvent and a soluble polymer, a BNNT, and a library molecule with an affinity for a BNNT of <1E−7 M.

A2.2.7.5: A DNA-encoded macrocycle, a medium comprising an organic solvent and a soluble polymer, a BNNT, and a library molecule with an affinity for a BNNT of <1E−8 M.

A2.2.7.6: A DNA-encoded macrocycle, a medium comprising an organic solvent and a soluble polymer, a BNNT, and a library molecule with an affinity for a BNNT of <1E−9 M.

A2.2.7.7: A DNA-encoded macrocycle, a medium comprising an organic solvent and a soluble polymer, a BNNT, and a library molecule with an affinity for a BNNT of <1E−10 M.

A2.2.7.8: A DNA-encoded macrocycle, a medium comprising an organic solvent and a soluble polymer, a BNNT, and a library molecule with an affinity for a BNNT of <1E−12 M.

A2.3.1.1: A DNA-encoded macrocycle, a medium comprising a soluble polymer, a CNT, and a library molecule with an affinity for a CNT of <1E−2 M.

A2.3.1.2: A DNA-encoded macrocycle, a medium comprising a soluble polymer, a CNT, and a library molecule with an affinity for a CNT of <1E−4 M.

A2.3.1.3: A DNA-encoded macrocycle, a medium comprising a soluble polymer, a CNT, and a library molecule with an affinity for a CNT of <1E−6 M.

A2.3.1.4: A DNA-encoded macrocycle, a medium comprising a soluble polymer, a CNT, and a library molecule with an affinity for a CNT of <1E−7 M.

A2.3.1.5: A DNA-encoded macrocycle, a medium comprising a soluble polymer, a CNT, and a library molecule with an affinity for a CNT of <1E−8 M.

A2.3.1.6: A DNA-encoded macrocycle, a medium comprising a soluble polymer, a CNT, and a library molecule with an affinity for a CNT of <1E−9 M.

A2.3.1.7: A DNA-encoded macrocycle, a medium comprising a soluble polymer, a CNT, and a library molecule with an affinity for a CNT of <1E−10 M.

A2.3.1.8: A DNA-encoded macrocycle, a medium comprising a soluble polymer, a CNT, and a library molecule with an affinity for a CNT of <1E−12 M.

A2.3.2.1: A DNA-encoded macrocycle, a medium comprising a soluble polymer, a GS, and a library molecule with an affinity for a GS of <1E−2 M.

A2.3.2.2: A DNA-encoded macrocycle, a medium comprising a soluble polymer, a GS, and a library molecule with an affinity for a GS of <1E−4 M.

A2.3.2.3: A DNA-encoded macrocycle, a medium comprising a soluble polymer, a GS, and a library molecule with an affinity for a GS of <1E−6 M.

A2.3.2.4: A DNA-encoded macrocycle, a medium comprising a soluble polymer, a GS, and a library molecule with an affinity for a GS of <1E−7 M.

A2.3.2.5: A DNA-encoded macrocycle, a medium comprising a soluble polymer, a GS, and a library molecule with an affinity for a GS of <1E−8 M.

A2.3.2.6: A DNA-encoded macrocycle, a medium comprising a soluble polymer, a GS, and a library molecule with an affinity for a GS of <1E−9 M.

A2.3.2.7: A DNA-encoded macrocycle, a medium comprising a soluble polymer, a GS, and a library molecule with an affinity for a GS of <1E−10 M.

A2.3.2.8: A DNA-encoded macrocycle, a medium comprising a soluble polymer, a GS, and a library molecule with an affinity for a GS of <1E−12 M.

A2.3.3.1: A DNA-encoded macrocycle, a medium comprising a soluble polymer, a SWNT, and a library molecule with an affinity for a SWNT of <1E−2 M.

A2.3.3.2: A DNA-encoded macrocycle, a medium comprising a soluble polymer, a SWNT, and a library molecule with an affinity for a SWNT of <1E−4 M.

A2.3.3.3: A DNA-encoded macrocycle, a medium comprising a soluble polymer, a SWNT, and a library molecule with an affinity for a SWNT of <1E−6 M.

A2.3.3.4: A DNA-encoded macrocycle, a medium comprising a soluble polymer, a SWNT, and a library molecule with an affinity for a SWNT of <1E−7 M.

A2.3.3.5: A DNA-encoded macrocycle, a medium comprising a soluble polymer, a SWNT, and a library molecule with an affinity for a SWNT of <1E−8 M.

A2.3.3.6: A DNA-encoded macrocycle, a medium comprising a soluble polymer, a SWNT, and a library molecule with an affinity for a SWNT of <1E−9 M.

A2.3.3.7: A DNA-encoded macrocycle, a medium comprising a soluble polymer, a SWNT, and a library molecule with an affinity for a SWNT of <1E−10 M.

A2.3.3.8: A DNA-encoded macrocycle, a medium comprising a soluble polymer, a SWNT, and a library molecule with an affinity for a SWNT of <1E−12 M.

A2.3.4.1: A DNA-encoded macrocycle, a medium comprising a soluble polymer, a MWNT, and a library molecule with an affinity for a MWNT of <1E−2 M.

A2.3.4.2: A DNA-encoded macrocycle, a medium comprising a soluble polymer, a MWNT, and a library molecule with an affinity for a MWNT of <1E−4 M.

A2.3.4.3: A DNA-encoded macrocycle, a medium comprising a soluble polymer, a MWNT, and a library molecule with an affinity for a MWNT of <1E−6 M.

A2.3.4.4: A DNA-encoded macrocycle, a medium comprising a soluble polymer, a MWNT, and a library molecule with an affinity for a MWNT of <1E−7 M.

A2.3.4.5: A DNA-encoded macrocycle, a medium comprising a soluble polymer, a MWNT, and a library molecule with an affinity for a MWNT of <1E−8 M.

A2.3.4.6: A DNA-encoded macrocycle, a medium comprising a soluble polymer, a MWNT, and a library molecule with an affinity for a MWNT of <1E−9 M.

A2.3.4.7: A DNA-encoded macrocycle, a medium comprising a soluble polymer, a MWNT, and a library molecule with an affinity for a MWNT of <1E−10 M.

A2.3.4.8: A DNA-encoded macrocycle, a medium comprising a soluble polymer, a MWNT, and a library molecule with an affinity for a MWNT of <1E−12 M.

A2.3.5.1: A DNA-encoded macrocycle, a medium comprising a soluble polymer, a nanotube, and a library molecule with an affinity for a nanotube of <1E−2 M.

A2.3.5.2: A DNA-encoded macrocycle, a medium comprising a soluble polymer, a nanotube, and a library molecule with an affinity for a nanotube of <1E−4 M.

A2.3.5.3: A DNA-encoded macrocycle, a medium comprising a soluble polymer, a nanotube, and a library molecule with an affinity for a nanotube of <1E−6 M.

A2.3.5.4: A DNA-encoded macrocycle, a medium comprising a soluble polymer, a nanotube, and a library molecule with an affinity for a nanotube of <1E−7 M.

A2.3.5.5: A DNA-encoded macrocycle, a medium comprising a soluble polymer, a nanotube, and a library molecule with an affinity for a nanotube of <1E−8 M.

A2.3.5.6: A DNA-encoded macrocycle, a medium comprising a soluble polymer, a nanotube, and a library molecule with an affinity for a nanotube of <1E−9 M.

A2.3.5.7: A DNA-encoded macrocycle, a medium comprising a soluble polymer, a nanotube, and a library molecule with an affinity for a nanotube of <1E−10 M.

A2.3.5.8: A DNA-encoded macrocycle, a medium comprising a soluble polymer, a nanotube, and a library molecule with an affinity for a nanotube of <1E−12 M.

A2.3.6.1: A DNA-encoded macrocycle, a medium comprising a soluble polymer, a fullerene, and a library molecule with an affinity for a fullerene of <1E−2 M.

A2.3.6.2: A DNA-encoded macrocycle, a medium comprising a soluble polymer, a fullerene, and a library molecule with an affinity for a fullerene of <1E−4 M.

A2.3.6.3: A DNA-encoded macrocycle, a medium comprising a soluble polymer, a fullerene, and a library molecule with an affinity for a fullerene of <1E−6 M.

A2.3.6.4: A DNA-encoded macrocycle, a medium comprising a soluble polymer, a fullerene, and a library molecule with an affinity for a fullerene of <1E−7 M.

A2.3.6.5: A DNA-encoded macrocycle, a medium comprising a soluble polymer, a fullerene, and a library molecule with an affinity for a fullerene of <1E−8 M.

A2.3.6.6: A DNA-encoded macrocycle, a medium comprising a soluble polymer, a fullerene, and a library molecule with an affinity for a fullerene of <1E−9 M.

A2.3.6.7: A DNA-encoded macrocycle, a medium comprising a soluble polymer, a fullerene, and a library molecule with an affinity for a fullerene of <1E−10 M.

A2.3.6.8: A DNA-encoded macrocycle, a medium comprising a soluble polymer, a fullerene, and a library molecule with an affinity for a fullerene of <1E−12 M.

A2.3.7.1: A DNA-encoded macrocycle, a medium comprising a soluble polymer, a BNNT, and a library molecule with an affinity for a BNNT of <1E−2 M.

A2.3.7.2: A DNA-encoded macrocycle, a medium comprising a soluble polymer, a BNNT, and a library molecule with an affinity for a BNNT of <1E−4 M.

A2.3.7.3: A DNA-encoded macrocycle, a medium comprising a soluble polymer, a BNNT, and a library molecule with an affinity for a BNNT of <1E−6 M.

A2.3.7.4: A DNA-encoded macrocycle, a medium comprising a soluble polymer, a BNNT, and a library molecule with an affinity for a BNNT of <1E−7 M.

A2.3.7.5: A DNA-encoded macrocycle, a medium comprising a soluble polymer, a BNNT, and a library molecule with an affinity for a BNNT of <1E−8 M.

A2.3.7.6: A DNA-encoded macrocycle, a medium comprising a soluble polymer, a BNNT, and a library molecule with an affinity for a BNNT of <1E−9 M.

A2.3.7.7: A DNA-encoded macrocycle, a medium comprising a soluble polymer, a BNNT, and a library molecule with an affinity for a BNNT of <1E−10 M.

A2.3.7.8: A DNA-encoded macrocycle, a medium comprising a soluble polymer, a BNNT, and a library molecule with an affinity for a BNNT of <1E−12 M.

A2.4.1.1: A DNA-encoded macrocycle, a medium comprising >50% organic solvent, a CNT, and a library molecule with an affinity for a CNT of <1E−2 M.

A2.4.1.2: A DNA-encoded macrocycle, a medium comprising >50% organic solvent, a CNT, and a library molecule with an affinity for a CNT of <1E−4 M.

A2.4.1.3: A DNA-encoded macrocycle, a medium comprising >50% organic solvent, a CNT, and a library molecule with an affinity for a CNT of <1E−6 M.

A2.4.1.4: A DNA-encoded macrocycle, a medium comprising >50% organic solvent, a CNT, and a library molecule with an affinity for a CNT of <1E−7 M.

A2.4.1.5: A DNA-encoded macrocycle, a medium comprising >50% organic solvent, a CNT, and a library molecule with an affinity for a CNT of <1E−8 M.

A2.4.1.6: A DNA-encoded macrocycle, a medium comprising >50% organic solvent, a CNT, and a library molecule with an affinity for a CNT of <1E−9 M.

A2.4.1.7: A DNA-encoded macrocycle, a medium comprising >50% organic solvent, a CNT, and a library molecule with an affinity for a CNT of <1E−10 M.

A2.4.1.8: A DNA-encoded macrocycle, a medium comprising >50% organic solvent, a CNT, and a library molecule with an affinity for a CNT of <1E−12 M.

A2.4.2.1: A DNA-encoded macrocycle, a medium comprising >50% organic solvent, a GS, and a library molecule with an affinity for a GS of <1E−2 M.

A2.4.2.2: A DNA-encoded macrocycle, a medium comprising >50% organic solvent, a GS, and a library molecule with an affinity for a GS of <1E−4 M.

A2.4.2.3: A DNA-encoded macrocycle, a medium comprising >50% organic solvent, a GS, and a library molecule with an affinity for a GS of <1E−6 M.

A2.4.2.4: A DNA-encoded macrocycle, a medium comprising >50% organic solvent, a GS, and a library molecule with an affinity for a GS of <1E−7 M.

A2.4.2.5: A DNA-encoded macrocycle, a medium comprising >50% organic solvent, a GS, and a library molecule with an affinity for a GS of <1E−8 M.

A2.4.2.6: A DNA-encoded macrocycle, a medium comprising >50% organic solvent, a GS, and a library molecule with an affinity for a GS of <1E−9 M.

A2.4.2.7: A DNA-encoded macrocycle, a medium comprising >50% organic solvent, a GS, and a library molecule with an affinity for a GS of <1E−10 M.

A2.4.2.8: A DNA-encoded macrocycle, a medium comprising >50% organic solvent, a GS, and a library molecule with an affinity for a GS of <1E−12 M.

A2.4.3.1: A DNA-encoded macrocycle, a medium comprising >50% organic solvent, a SWNT, and a library molecule with an affinity for a SWNT of <1E−2 M.

A2.4.3.2: A DNA-encoded macrocycle, a medium comprising >50% organic solvent, a SWNT, and a library molecule with an affinity for a SWNT of <1E−4 M.

A2.4.3.3: A DNA-encoded macrocycle, a medium comprising >50% organic solvent, a SWNT, and a library molecule with an affinity for a SWNT of <1E−6 M.

A2.4.3.4: A DNA-encoded macrocycle, a medium comprising >50% organic solvent, a SWNT, and a library molecule with an affinity for a SWNT of <1E−7 M.

A2.4.3.5: A DNA-encoded macrocycle, a medium comprising >50% organic solvent, a SWNT, and a library molecule with an affinity for a SWNT of <1E−8 M.

A2.4.3.6: A DNA-encoded macrocycle, a medium comprising >50% organic solvent, a SWNT, and a library molecule with an affinity for a SWNT of <1E−9 M.

A2.4.3.7: A DNA-encoded macrocycle, a medium comprising >50% organic solvent, a SWNT, and a library molecule with an affinity for a SWNT of <1E−10 M.

A2.4.3.8: A DNA-encoded macrocycle, a medium comprising >50% organic solvent, a SWNT, and a library molecule with an affinity for a SWNT of <1E−12 M.

A2.4.4.1: A DNA-encoded macrocycle, a medium comprising >50% organic solvent, a MWNT, and a library molecule with an affinity for a MWNT of <1E−2 M.

A2.4.4.2: A DNA-encoded macrocycle, a medium comprising >50% organic solvent, a MWNT, and a library molecule with an affinity for a MWNT of <1E−4 M.

A2.4.4.3: A DNA-encoded macrocycle, a medium comprising >50% organic solvent, a MWNT, and a library molecule with an affinity for a MWNT of <1E−6 M.

A2.4.4.4: A DNA-encoded macrocycle, a medium comprising >50% organic solvent, a MWNT, and a library molecule with an affinity for a MWNT of <1E−7 M.

A2.4.4.5: A DNA-encoded macrocycle, a medium comprising >50% organic solvent, a MWNT, and a library molecule with an affinity for a MWNT of <1E−8 M.

A2.4.4.6: A DNA-encoded macrocycle, a medium comprising >50% organic solvent, a MWNT, and a library molecule with an affinity for a MWNT of <1E−9 M.

A2.4.4.7: A DNA-encoded macrocycle, a medium comprising >50% organic solvent, a MWNT, and a library molecule with an affinity for a MWNT of <1E−10 M.

A2.4.4.8: A DNA-encoded macrocycle, a medium comprising >50% organic solvent, a MWNT, and a library molecule with an affinity for a MWNT of <1E−12 M.

A2.4.5.1: A DNA-encoded macrocycle, a medium comprising >50% organic solvent, a nanotube, and a library molecule with an affinity for a nanotube of <1E−2 M.

A2.4.5.2: A DNA-encoded macrocycle, a medium comprising >50% organic solvent, a nanotube, and a library molecule with an affinity for a nanotube of <1E−4 M.

A2.4.5.3: A DNA-encoded macrocycle, a medium comprising >50% organic solvent, a nanotube, and a library molecule with an affinity for a nanotube of <1E−6 M.

A2.4.5.4: A DNA-encoded macrocycle, a medium comprising >50% organic solvent, a nanotube, and a library molecule with an affinity for a nanotube of <1E−7 M.

A2.4.5.5: A DNA-encoded macrocycle, a medium comprising >50% organic solvent, a nanotube, and a library molecule with an affinity for a nanotube of <1E−8 M.

A2.4.5.6: A DNA-encoded macrocycle, a medium comprising >50% organic solvent, a nanotube, and a library molecule with an affinity for a nanotube of <1E−9 M.

A2.4.5.7: A DNA-encoded macrocycle, a medium comprising >50% organic solvent, a nanotube, and a library molecule with an affinity for a nanotube of <1E−10 M.

A2.4.5.8: A DNA-encoded macrocycle, a medium comprising >50% organic solvent, a nanotube, and a library molecule with an affinity for a nanotube of <1E−12 M.

A2.4.6.1: A DNA-encoded macrocycle, a medium comprising >50% organic solvent, a fullerene, and a library molecule with an affinity for a fullerene of <1E−2 M.

A2.4.6.2: A DNA-encoded macrocycle, a medium comprising >50% organic solvent, a fullerene, and a library molecule with an affinity for a fullerene of <1E−4 M.

A2.4.6.3: A DNA-encoded macrocycle, a medium comprising >50% organic solvent, a fullerene, and a library molecule with an affinity for a fullerene of <1E−6 M.

A2.4.6.4: A DNA-encoded macrocycle, a medium comprising >50% organic solvent, a fullerene, and a library molecule with an affinity for a fullerene of <1E−7 M.

A2.4.6.5: A DNA-encoded macrocycle, a medium comprising >50% organic solvent, a fullerene, and a library molecule with an affinity for a fullerene of <1E−8 M.

A2.4.6.6: A DNA-encoded macrocycle, a medium comprising >50% organic solvent, a fullerene, and a library molecule with an affinity for a fullerene of <1E−9 M.

A2.4.6.7: A DNA-encoded macrocycle, a medium comprising >50% organic solvent, a fullerene, and a library molecule with an affinity for a fullerene of <1E−10 M.

A2.4.6.8: A DNA-encoded macrocycle, a medium comprising >50% organic solvent, a fullerene, and a library molecule with an affinity for a fullerene of <1E−12 M.

A2.4.7.1: A DNA-encoded macrocycle, a medium comprising >50% organic solvent, a BNNT, and a library molecule with an affinity for a BNNT of <1E−2 M.

A2.4.7.2: A DNA-encoded macrocycle, a medium comprising >50% organic solvent, a BNNT, and a library molecule with an affinity for a BNNT of <1E−4 M.

A2.4.7.3: A DNA-encoded macrocycle, a medium comprising >50% organic solvent, a BNNT, and a library molecule with an affinity for a BNNT of <1E−6 M.

A2.4.7.4: A DNA-encoded macrocycle, a medium comprising >50% organic solvent, a BNNT, and a library molecule with an affinity for a BNNT of <1E−7 M.

A2.4.7.5: A DNA-encoded macrocycle, a medium comprising >50% organic solvent, a BNNT, and a library molecule with an affinity for a BNNT of <1E−8 M.

A2.4.7.6: A DNA-encoded macrocycle, a medium comprising >50% organic solvent, a BNNT, and a library molecule with an affinity for a BNNT of <1E−9 M.

A2.4.7.7: A DNA-encoded macrocycle, a medium comprising >50% organic solvent, a BNNT, and a library molecule with an affinity for a BNNT of <1E−10 M.

A2.4.7.8: A DNA-encoded macrocycle, a medium comprising >50% organic solvent, a BNNT, and a library molecule with an affinity for a BNNT of <1E−12 M.

A2.5.1.1: A DNA-encoded macrocycle, a medium comprising >50% organic solvent and a soluble polymer, a CNT, and a library molecule with an affinity for a CNT of <1E−2 M.

A2.5.1.2: A DNA-encoded macrocycle, a medium comprising >50% organic solvent and a soluble polymer, a CNT, and a library molecule with an affinity for a CNT of <1E−4 M.

A2.5.1.3: A DNA-encoded macrocycle, a medium comprising >50% organic solvent and a soluble polymer, a CNT, and a library molecule with an affinity for a CNT of <1E−6 M.

A2.5.1.4: A DNA-encoded macrocycle, a medium comprising >50% organic solvent and a soluble polymer, a CNT, and a library molecule with an affinity for a CNT of <1E−7 M.

A2.5.1.5: A DNA-encoded macrocycle, a medium comprising >50% organic solvent and a soluble polymer, a CNT, and a library molecule with an affinity for a CNT of <1E−8 M.

A2.5.1.6: A DNA-encoded macrocycle, a medium comprising >50% organic solvent and a soluble polymer, a CNT, and a library molecule with an affinity for a CNT of <1E−9 M.

A2.5.1.7: A DNA-encoded macrocycle, a medium comprising >50% organic solvent and a soluble polymer, a CNT, and a library molecule with an affinity for a CNT of <1E−10 M.

A2.5.1.8: A DNA-encoded macrocycle, a medium comprising >50% organic solvent and a soluble polymer, a CNT, and a library molecule with an affinity for a CNT of <1E−12 M.

A2.5.2.1: A DNA-encoded macrocycle, a medium comprising >50% organic solvent and a soluble polymer, a GS, and a library molecule with an affinity for a GS of <1E−2 M.

A2.5.2.2: A DNA-encoded macrocycle, a medium comprising >50% organic solvent and a soluble polymer, a GS, and a library molecule with an affinity for a GS of <1E−4 M.

A2.5.2.3: A DNA-encoded macrocycle, a medium comprising >50% organic solvent and a soluble polymer, a GS, and a library molecule with an affinity for a GS of <1E−6 M.

A2.5.2.4: A DNA-encoded macrocycle, a medium comprising >50% organic solvent and a soluble polymer, a GS, and a library molecule with an affinity for a GS of <1E−7 M.

A2.5.2.5: A DNA-encoded macrocycle, a medium comprising >50% organic solvent and a soluble polymer, a GS, and a library molecule with an affinity for a GS of <1E−8 M.

A2.5.2.6: A DNA-encoded macrocycle, a medium comprising >50% organic solvent and a soluble polymer, a GS, and a library molecule with an affinity for a GS of <1E−9 M.

A2.5.2.7: A DNA-encoded macrocycle, a medium comprising >50% organic solvent and a soluble polymer, a GS, and a library molecule with an affinity for a GS of <1E−10 M.

A2.5.2.8: A DNA-encoded macrocycle, a medium comprising >50% organic solvent and a soluble polymer, a GS, and a library molecule with an affinity for a GS of <1E−12 M.

A2.5.3.1: A DNA-encoded macrocycle, a medium comprising >50% organic solvent and a soluble polymer, a SWNT, and a library molecule with an affinity for a SWNT of <1E−2 M.

A2.5.3.2: A DNA-encoded macrocycle, a medium comprising >50% organic solvent and a soluble polymer, a SWNT, and a library molecule with an affinity for a SWNT of <1E−4 M.

A2.5.3.3: A DNA-encoded macrocycle, a medium comprising >50% organic solvent and a soluble polymer, a SWNT, and a library molecule with an affinity for a SWNT of <1E−6 M.

A2.5.3.4: A DNA-encoded macrocycle, a medium comprising >50% organic solvent and a soluble polymer, a SWNT, and a library molecule with an affinity for a SWNT of <1E−7 M.

A2.5.3.5: A DNA-encoded macrocycle, a medium comprising >50% organic solvent and a soluble polymer, a SWNT, and a library molecule with an affinity for a SWNT of <1E−8 M.

A2.5.3.6: A DNA-encoded macrocycle, a medium comprising >50% organic solvent and a soluble polymer, a SWNT, and a library molecule with an affinity for a SWNT of <1E−9 M.

A2.5.3.7: A DNA-encoded macrocycle, a medium comprising >50% organic solvent and a soluble polymer, a SWNT, and a library molecule with an affinity for a SWNT of <1E−10 M.

A2.5.3.8: A DNA-encoded macrocycle, a medium comprising >50% organic solvent and a soluble polymer, a SWNT, and a library molecule with an affinity for a SWNT of <1E−12 M.

A2.5.4.1: A DNA-encoded macrocycle, a medium comprising >50% organic solvent and a soluble polymer, a MWNT, and a library molecule with an affinity for a MWNT of <1E−2 M.

A2.5.4.2: A DNA-encoded macrocycle, a medium comprising >50% organic solvent and a soluble polymer, a MWNT, and a library molecule with an affinity for a MWNT of <1E−4 M.

A2.5.4.3: A DNA-encoded macrocycle, a medium comprising >50% organic solvent and a soluble polymer, a MWNT, and a library molecule with an affinity for a MWNT of <1E−6 M.

A2.5.4.4: A DNA-encoded macrocycle, a medium comprising >50% organic solvent and a soluble polymer, a MWNT, and a library molecule with an affinity for a MWNT of <1E−7 M.

A2.5.4.5: A DNA-encoded macrocycle, a medium comprising >50% organic solvent and a soluble polymer, a MWNT, and a library molecule with an affinity for a MWNT of <1E−8 M.

A2.5.4.6: A DNA-encoded macrocycle, a medium comprising >50% organic solvent and a soluble polymer, a MWNT, and a library molecule with an affinity for a MWNT of <1E−9 M.

A2.5.4.7: A DNA-encoded macrocycle, a medium comprising >50% organic solvent and a soluble polymer, a MWNT, and a library molecule with an affinity for a MWNT of <1E−10 M.

A2.5.4.8: A DNA-encoded macrocycle, a medium comprising >50% organic solvent and a soluble polymer, a MWNT, and a library molecule with an affinity for a MWNT of <1E−12 M.

A2.5.5.1: A DNA-encoded macrocycle, a medium comprising >50% organic solvent and a soluble polymer, a nanotube, and a library molecule with an affinity for a nanotube of <1E−2 M.

A2.5.5.2: A DNA-encoded macrocycle, a medium comprising >50% organic solvent and a soluble polymer, a nanotube, and a library molecule with an affinity for a nanotube of <1E−4 M.

A2.5.5.3: A DNA-encoded macrocycle, a medium comprising >50% organic solvent and a soluble polymer, a nanotube, and a library molecule with an affinity for a nanotube of <1E−6 M.

A2.5.5.4: A DNA-encoded macrocycle, a medium comprising >50% organic solvent and a soluble polymer, a nanotube, and a library molecule with an affinity for a nanotube of <1E−7 M.

A2.5.5.5: A DNA-encoded macrocycle, a medium comprising >50% organic solvent and a soluble polymer, a nanotube, and a library molecule with an affinity for a nanotube of <1E−8 M.

A2.5.5.6: A DNA-encoded macrocycle, a medium comprising >50% organic solvent and a soluble polymer, a nanotube, and a library molecule with an affinity for a nanotube of <1E−9 M.

A2.5.5.7: A DNA-encoded macrocycle, a medium comprising >50% organic solvent and a soluble polymer, a nanotube, and a library molecule with an affinity for a nanotube of <1E−10 M.

A2.5.5.8: A DNA-encoded macrocycle, a medium comprising >50% organic solvent and a soluble polymer, a nanotube, and a library molecule with an affinity for a nanotube of <1E−12 M.

A2.5.6.1: A DNA-encoded macrocycle, a medium comprising >50% organic solvent and a soluble polymer, a fullerene, and a library molecule with an affinity for a fullerene of <1E−2 M.

A2.5.6.2: A DNA-encoded macrocycle, a medium comprising >50% organic solvent and a soluble polymer, a fullerene, and a library molecule with an affinity for a fullerene of <1E−4 M.

A2.5.6.3: A DNA-encoded macrocycle, a medium comprising >50% organic solvent and a soluble polymer, a fullerene, and a library molecule with an affinity for a fullerene of <1E−6 M.

A2.5.6.4: A DNA-encoded macrocycle, a medium comprising >50% organic solvent and a soluble polymer, a fullerene, and a library molecule with an affinity for a fullerene of <1E−7 M.

A2.5.6.5: A DNA-encoded macrocycle, a medium comprising >50% organic solvent and a soluble polymer, a fullerene, and a library molecule with an affinity for a fullerene of <1E−8 M.

A2.5.6.6: A DNA-encoded macrocycle, a medium comprising >50% organic solvent and a soluble polymer, a fullerene, and a library molecule with an affinity for a fullerene of <1E−9 M.

A2.5.6.7: A DNA-encoded macrocycle, a medium comprising >50% organic solvent and a soluble polymer, a fullerene, and a library molecule with an affinity for a fullerene of <1E−10 M.

A2.5.6.8: A DNA-encoded macrocycle, a medium comprising >50% organic solvent and a soluble polymer, a fullerene, and a library molecule with an affinity for a fullerene of <1E−12 M.

A2.5.7.1: A DNA-encoded macrocycle, a medium comprising >50% organic solvent and a soluble polymer, a BNNT, and a library molecule with an affinity for a BNNT of <1E−2 M.

A2.5.7.2: A DNA-encoded macrocycle, a medium comprising >50% organic solvent and a soluble polymer, a BNNT, and a library molecule with an affinity for a BNNT of <1E−4 M.

A2.5.7.3: A DNA-encoded macrocycle, a medium comprising >50% organic solvent and a soluble polymer, a BNNT, and a library molecule with an affinity for a BNNT of <1E−6 M.

A2.5.7.4: A DNA-encoded macrocycle, a medium comprising >50% organic solvent and a soluble polymer, a BNNT, and a library molecule with an affinity for a BNNT of <1E−7 M.

A2.5.7.5: A DNA-encoded macrocycle, a medium comprising >50% organic solvent and a soluble polymer, a BNNT, and a library molecule with an affinity for a BNNT of <1E−8 M.

A2.5.7.6: A DNA-encoded macrocycle, a medium comprising >50% organic solvent and a soluble polymer, a BNNT, and a library molecule with an affinity for a BNNT of <1E−9 M.

A2.5.7.7: A DNA-encoded macrocycle, a medium comprising >50% organic solvent and a soluble polymer, a BNNT, and a library molecule with an affinity for a BNNT of <1E−10 M.

A2.5.7.8: A DNA-encoded macrocycle, a medium comprising >50% organic solvent and a soluble polymer, a BNNT, and a library molecule with an affinity for a BNNT of <1E−12 M.

A2.6.1.1: A DNA-encoded macrocycle, a medium comprising >80% organic solvent, a CNT, and a library molecule with an affinity for a CNT of <1E−2 M.

A2.6.1.2: A DNA-encoded macrocycle, a medium comprising >80% organic solvent, a CNT, and a library molecule with an affinity for a CNT of <1E−4 M.

A2.6.1.3: A DNA-encoded macrocycle, a medium comprising >80% organic solvent, a CNT, and a library molecule with an affinity for a CNT of <1E−6 M.

A2.6.1.4: A DNA-encoded macrocycle, a medium comprising >80% organic solvent, a CNT, and a library molecule with an affinity for a CNT of <1E−7 M.

A2.6.1.5: A DNA-encoded macrocycle, a medium comprising >80% organic solvent, a CNT, and a library molecule with an affinity for a CNT of <1E−8 M.

A2.6.1.6: A DNA-encoded macrocycle, a medium comprising >80% organic solvent, a CNT, and a library molecule with an affinity for a CNT of <1E−9 M.

A2.6.1.7: A DNA-encoded macrocycle, a medium comprising >80% organic solvent, a CNT, and a library molecule with an affinity for a CNT of <1E−10 M.

A2.6.1.8: A DNA-encoded macrocycle, a medium comprising >80% organic solvent, a CNT, and a library molecule with an affinity for a CNT of <1E−12 M.

A2.6.2.1: A DNA-encoded macrocycle, a medium comprising >80% organic solvent, a GS, and a library molecule with an affinity for a GS of <1E−2 M.

A2.6.2.2: A DNA-encoded macrocycle, a medium comprising >80% organic solvent, a GS, and a library molecule with an affinity for a GS of <1E−4 M.

A2.6.2.3: A DNA-encoded macrocycle, a medium comprising >80% organic solvent, a GS, and a library molecule with an affinity for a GS of <1E−6 M.

A2.6.2.4: A DNA-encoded macrocycle, a medium comprising >80% organic solvent, a GS, and a library molecule with an affinity for a GS of <1E−7 M.

A2.6.2.5: A DNA-encoded macrocycle, a medium comprising >80% organic solvent, a GS, and a library molecule with an affinity for a GS of <1E−8 M.

A2.6.2.6: A DNA-encoded macrocycle, a medium comprising >80% organic solvent, a GS, and a library molecule with an affinity for a GS of <1E−9 M.

A2.6.2.7: A DNA-encoded macrocycle, a medium comprising >80% organic solvent, a GS, and a library molecule with an affinity for a GS of <1E−10 M.

A2.6.2.8: A DNA-encoded macrocycle, a medium comprising >80% organic solvent, a GS, and a library molecule with an affinity for a GS of <1E−12 M.

A2.6.3.1: A DNA-encoded macrocycle, a medium comprising >80% organic solvent, a SWNT, and a library molecule with an affinity for a SWNT of <1E−2 M.

A2.6.3.2: A DNA-encoded macrocycle, a medium comprising >80% organic solvent, a SWNT, and a library molecule with an affinity for a SWNT of <1E−4 M.

A2.6.3.3: A DNA-encoded macrocycle, a medium comprising >80% organic solvent, a SWNT, and a library molecule with an affinity for a SWNT of <1E−6 M.

A2.6.3.4: A DNA-encoded macrocycle, a medium comprising >80% organic solvent, a SWNT, and a library molecule with an affinity for a SWNT of <1E−7 M.

A2.6.3.5: A DNA-encoded macrocycle, a medium comprising >80% organic solvent, a SWNT, and a library molecule with an affinity for a SWNT of <1E−8 M.

A2.6.3.6: A DNA-encoded macrocycle, a medium comprising >80% organic solvent, a SWNT, and a library molecule with an affinity for a SWNT of <1E−9 M.

A2.6.3.7: A DNA-encoded macrocycle, a medium comprising >80% organic solvent, a SWNT, and a library molecule with an affinity for a SWNT of <1E−10 M.

A2.6.3.8: A DNA-encoded macrocycle, a medium comprising >80% organic solvent, a SWNT, and a library molecule with an affinity for a SWNT of <1E−12 M.

A2.6.4.1: A DNA-encoded macrocycle, a medium comprising >80% organic solvent, a MWNT, and a library molecule with an affinity for a MWNT of <1E−2 M.

A2.6.4.2: A DNA-encoded macrocycle, a medium comprising >80% organic solvent, a MWNT, and a library molecule with an affinity for a MWNT of <1E−4 M.

A2.6.4.3: A DNA-encoded macrocycle, a medium comprising >80% organic solvent, a MWNT, and a library molecule with an affinity for a MWNT of <1E−6 M.

A2.6.4.4: A DNA-encoded macrocycle, a medium comprising >80% organic solvent, a MWNT, and a library molecule with an affinity for a MWNT of <1E−7 M.

A2.6.4.5: A DNA-encoded macrocycle, a medium comprising >80% organic solvent, a MWNT, and a library molecule with an affinity for a MWNT of <1E−8 M.

A2.6.4.6: A DNA-encoded macrocycle, a medium comprising >80% organic solvent, a MWNT, and a library molecule with an affinity for a MWNT of <1E−9 M.

A2.6.4.7: A DNA-encoded macrocycle, a medium comprising >80% organic solvent, a MWNT, and a library molecule with an affinity for a MWNT of <1E−10 M.

A2.6.4.8: A DNA-encoded macrocycle, a medium comprising >80% organic solvent, a MWNT, and a library molecule with an affinity for a MWNT of <1E−12 M.

A2.6.5.1: A DNA-encoded macrocycle, a medium comprising >80% organic solvent, a nanotube, and a library molecule with an affinity for a nanotube of <1E−2 M.

A2.6.5.2: A DNA-encoded macrocycle, a medium comprising >80% organic solvent, a nanotube, and a library molecule with an affinity for a nanotube of <1E−4 M.

A2.6.5.3: A DNA-encoded macrocycle, a medium comprising >80% organic solvent, a nanotube, and a library molecule with an affinity for a nanotube of <1E−6 M.

A2.6.5.4: A DNA-encoded macrocycle, a medium comprising >80% organic solvent, a nanotube, and a library molecule with an affinity for a nanotube of <1E−7 M.

A2.6.5.5: A DNA-encoded macrocycle, a medium comprising >80% organic solvent, a nanotube, and a library molecule with an affinity for a nanotube of <1E−8 M.

A2.6.5.6: A DNA-encoded macrocycle, a medium comprising >80% organic solvent, a nanotube, and a library molecule with an affinity for a nanotube of <1E−9 M.

A2.6.5.7: A DNA-encoded macrocycle, a medium comprising >80% organic solvent, a nanotube, and a library molecule with an affinity for a nanotube of <1E−10 M.

A2.6.5.8: A DNA-encoded macrocycle, a medium comprising >80% organic solvent, a nanotube, and a library molecule with an affinity for a nanotube of <1E−12 M.

A2.6.6.1: A DNA-encoded macrocycle, a medium comprising >80% organic solvent, a fullerene, and a library molecule with an affinity for a fullerene of <1E−2 M.

A2.6.6.2: A DNA-encoded macrocycle, a medium comprising >80% organic solvent, a fullerene, and a library molecule with an affinity for a fullerene of <1E−4 M.

A2.6.6.3: A DNA-encoded macrocycle, a medium comprising >80% organic solvent, a fullerene, and a library molecule with an affinity for a fullerene of <1E−6 M.

A2.6.6.4: A DNA-encoded macrocycle, a medium comprising >80% organic solvent, a fullerene, and a library molecule with an affinity for a fullerene of <1E−7 M.

A2.6.6.5: A DNA-encoded macrocycle, a medium comprising >80% organic solvent, a fullerene, and a library molecule with an affinity for a fullerene of <1E−8 M.

A2.6.6.6: A DNA-encoded macrocycle, a medium comprising >80% organic solvent, a fullerene, and a library molecule with an affinity for a fullerene of <1E−9 M.

A2.6.6.7: A DNA-encoded macrocycle, a medium comprising >80% organic solvent, a fullerene, and a library molecule with an affinity for a fullerene of <1E−10 M.

A2.6.6.8: A DNA-encoded macrocycle, a medium comprising >80% organic solvent, a fullerene, and a library molecule with an affinity for a fullerene of <1E−12 M.

A2.6.7.1: A DNA-encoded macrocycle, a medium comprising >80% organic solvent, a BNNT, and a library molecule with an affinity for a BNNT of <1E−2 M.

A2.6.7.2: A DNA-encoded macrocycle, a medium comprising >80% organic solvent, a BNNT, and a library molecule with an affinity for a BNNT of <1E−4 M.

A2.6.7.3: A DNA-encoded macrocycle, a medium comprising >80% organic solvent, a BNNT, and a library molecule with an affinity for a BNNT of <1E−6 M.

A2.6.7.4: A DNA-encoded macrocycle, a medium comprising >80% organic solvent, a BNNT, and a library molecule with an affinity for a BNNT of <1E−7 M.

A2.6.7.5: A DNA-encoded macrocycle, a medium comprising >80% organic solvent, a BNNT, and a library molecule with an affinity for a BNNT of <1E−8 M.

A2.6.7.6: A DNA-encoded macrocycle, a medium comprising >80% organic solvent, a BNNT, and a library molecule with an affinity for a BNNT of <1E−9 M.

A2.6.7.7: A DNA-encoded macrocycle, a medium comprising >80% organic solvent, a BNNT, and a library molecule with an affinity for a BNNT of <1E−10 M.

A2.6.7.8: A DNA-encoded macrocycle, a medium comprising >80% organic solvent, a BNNT, and a library molecule with an affinity for a BNNT of <1E−12 M.

A2.7.1.1: A DNA-encoded macrocycle, a medium comprising >80% organic solvent and a soluble polymer, a CNT, and a library molecule with an affinity for a CNT of <1E−2 M.

A2.7.1.2: A DNA-encoded macrocycle, a medium comprising >80% organic solvent and a soluble polymer, a CNT, and a library molecule with an affinity for a CNT of <1E−4 M.

A2.7.1.3: A DNA-encoded macrocycle, a medium comprising >80% organic solvent and a soluble polymer, a CNT, and a library molecule with an affinity for a CNT of <1E−6 M.

A2.7.1.4: A DNA-encoded macrocycle, a medium comprising >80% organic solvent and a soluble polymer, a CNT, and a library molecule with an affinity for a CNT of <1E−7 M.

A2.7.1.5: A DNA-encoded macrocycle, a medium comprising >80% organic solvent and a soluble polymer, a CNT, and a library molecule with an affinity for a CNT of <1E−8 M.

A2.7.1.6: A DNA-encoded macrocycle, a medium comprising >80% organic solvent and a soluble polymer, a CNT, and a library molecule with an affinity for a CNT of <1E−9 M.

A2.7.1.7: A DNA-encoded macrocycle, a medium comprising >80% organic solvent and a soluble polymer, a CNT, and a library molecule with an affinity for a CNT of <1E−10 M.

A2.7.1.8: A DNA-encoded macrocycle, a medium comprising >80% organic solvent and a soluble polymer, a CNT, and a library molecule with an affinity for a CNT of <1E−12 M.

A2.7.2.1: A DNA-encoded macrocycle, a medium comprising >80% organic solvent and a soluble polymer, a GS, and a library molecule with an affinity for a GS of <1E−2 M.

A2.7.2.2: A DNA-encoded macrocycle, a medium comprising >80% organic solvent and a soluble polymer, a GS, and a library molecule with an affinity for a GS of <1E−4 M.

A2.7.2.3: A DNA-encoded macrocycle, a medium comprising >80% organic solvent and a soluble polymer, a GS, and a library molecule with an affinity for a GS of <1E−6 M.

A2.7.2.4: A DNA-encoded macrocycle, a medium comprising >80% organic solvent and a soluble polymer, a GS, and a library molecule with an affinity for a GS of <1E−7 M.

A2.7.2.5: A DNA-encoded macrocycle, a medium comprising >80% organic solvent and a soluble polymer, a GS, and a library molecule with an affinity for a GS of <1E−8 M.

A2.7.2.6: A DNA-encoded macrocycle, a medium comprising >80% organic solvent and a soluble polymer, a GS, and a library molecule with an affinity for a GS of <1E−9 M.

A2.7.2.7: A DNA-encoded macrocycle, a medium comprising >80% organic solvent and a soluble polymer, a GS, and a library molecule with an affinity for a GS of <1E−10 M.

A2.7.2.8: A DNA-encoded macrocycle, a medium comprising >80% organic solvent and a soluble polymer, a GS, and a library molecule with an affinity for a GS of <1E−12 M.

A2.7.3.1: A DNA-encoded macrocycle, a medium comprising >80% organic solvent and a soluble polymer, a SWNT, and a library molecule with an affinity for a SWNT of <1E−2 M.

A2.7.3.2: A DNA-encoded macrocycle, a medium comprising >80% organic solvent and a soluble polymer, a SWNT, and a library molecule with an affinity for a SWNT of <1E−4 M.

A2.7.3.3: A DNA-encoded macrocycle, a medium comprising >80% organic solvent and a soluble polymer, a SWNT, and a library molecule with an affinity for a SWNT of <1E−6 M.

A2.7.3.4: A DNA-encoded macrocycle, a medium comprising >80% organic solvent and a soluble polymer, a SWNT, and a library molecule with an affinity for a SWNT of <1E−7 M.

A2.7.3.5: A DNA-encoded macrocycle, a medium comprising >80% organic solvent and a soluble polymer, a SWNT, and a library molecule with an affinity for a SWNT of <1E−8 M.

A2.7.3.6: A DNA-encoded macrocycle, a medium comprising >80% organic solvent and a soluble polymer, a SWNT, and a library molecule with an affinity for a SWNT of <1E−9 M.

A2.7.3.7: A DNA-encoded macrocycle, a medium comprising >80% organic solvent and a soluble polymer, a SWNT, and a library molecule with an affinity for a SWNT of <1E−10 M.

A2.7.3.8: A DNA-encoded macrocycle, a medium comprising >80% organic solvent and a soluble polymer, a SWNT, and a library molecule with an affinity for a SWNT of <1E−12 M.

A2.7.4.1: A DNA-encoded macrocycle, a medium comprising >80% organic solvent and a soluble polymer, a MWNT, and a library molecule with an affinity for a MWNT of <1E−2 M.

A2.7.4.2: A DNA-encoded macrocycle, a medium comprising >80% organic solvent and a soluble polymer, a MWNT, and a library molecule with an affinity for a MWNT of <1E−4 M.

A2.7.4.3: A DNA-encoded macrocycle, a medium comprising >80% organic solvent and a soluble polymer, a MWNT, and a library molecule with an affinity for a MWNT of <1E−6 M.

A2.7.4.4: A DNA-encoded macrocycle, a medium comprising >80% organic solvent and a soluble polymer, a MWNT, and a library molecule with an affinity for a MWNT of <1E−7 M.

A2.7.4.5: A DNA-encoded macrocycle, a medium comprising >80% organic solvent and a soluble polymer, a MWNT, and a library molecule with an affinity for a MWNT of <1E−8 M.

A2.7.4.6: A DNA-encoded macrocycle, a medium comprising >80% organic solvent and a soluble polymer, a MWNT, and a library molecule with an affinity for a MWNT of <1E−9 M.

A2.7.4.7: A DNA-encoded macrocycle, a medium comprising >80% organic solvent and a soluble polymer, a MWNT, and a library molecule with an affinity for a MWNT of <1E−10 M.

A2.7.4.8: A DNA-encoded macrocycle, a medium comprising >80% organic solvent and a soluble polymer, a MWNT, and a library molecule with an affinity for a MWNT of <1E−12 M.

A2.7.5.1: A DNA-encoded macrocycle, a medium comprising >80% organic solvent and a soluble polymer, a nanotube, and a library molecule with an affinity for a nanotube of <1E−2 M.

A2.7.5.2: A DNA-encoded macrocycle, a medium comprising >80% organic solvent and a soluble polymer, a nanotube, and a library molecule with an affinity for a nanotube of <1E−4 M.

A2.7.5.3: A DNA-encoded macrocycle, a medium comprising >80% organic solvent and a soluble polymer, a nanotube, and a library molecule with an affinity for a nanotube of <1E−6 M.

A2.7.5.4: A DNA-encoded macrocycle, a medium comprising >80% organic solvent and a soluble polymer, a nanotube, and a library molecule with an affinity for a nanotube of <1E−7 M.

A2.7.5.5: A DNA-encoded macrocycle, a medium comprising >80% organic solvent and a soluble polymer, a nanotube, and a library molecule with an affinity for a nanotube of <1E−8 M.

A2.7.5.6: A DNA-encoded macrocycle, a medium comprising >80% organic solvent and a soluble polymer, a nanotube, and a library molecule with an affinity for a nanotube of <1E−9 M.

A2.7.5.7: A DNA-encoded macrocycle, a medium comprising >80% organic solvent and a soluble polymer, a nanotube, and a library molecule with an affinity for a nanotube of <1E−10 M.

A2.7.5.8: A DNA-encoded macrocycle, a medium comprising >80% organic solvent and a soluble polymer, a nanotube, and a library molecule with an affinity for a nanotube of <1E−12 M.

A2.7.6.1: A DNA-encoded macrocycle, a medium comprising >80% organic solvent and a soluble polymer, a fullerene, and a library molecule with an affinity for a fullerene of <1E−2 M.

A2.7.6.2: A DNA-encoded macrocycle, a medium comprising >80% organic solvent and a soluble polymer, a fullerene, and a library molecule with an affinity for a fullerene of <1E−4 M.

A2.7.6.3: A DNA-encoded macrocycle, a medium comprising >80% organic solvent and a soluble polymer, a fullerene, and a library molecule with an affinity for a fullerene of <1E−6 M.

A2.7.6.4: A DNA-encoded macrocycle, a medium comprising >80% organic solvent and a soluble polymer, a fullerene, and a library molecule with an affinity for a fullerene of <1E−7 M.

A2.7.6.5: A DNA-encoded macrocycle, a medium comprising >80% organic solvent and a soluble polymer, a fullerene, and a library molecule with an affinity for a fullerene of <1E−8 M.

A2.7.6.6: A DNA-encoded macrocycle, a medium comprising >80% organic solvent and a soluble polymer, a fullerene, and a library molecule with an affinity for a fullerene of <1E−9 M.

A2.7.6.7: A DNA-encoded macrocycle, a medium comprising >80% organic solvent and a soluble polymer, a fullerene, and a library molecule with an affinity for a fullerene of <1E−10 M.

A2.7.6.8: A DNA-encoded macrocycle, a medium comprising >80% organic solvent and a soluble polymer, a fullerene, and a library molecule with an affinity for a fullerene of <1E−12 M.

A2.7.7.1: A DNA-encoded macrocycle, a medium comprising >80% organic solvent and a soluble polymer, a BNNT, and a library molecule with an affinity for a BNNT of <1E−2 M.

A2.7.7.2: A DNA-encoded macrocycle, a medium comprising >80% organic solvent and a soluble polymer, a BNNT, and a library molecule with an affinity for a BNNT of <1E−4 M.

A2.7.7.3: A DNA-encoded macrocycle, a medium comprising >80% organic solvent and a soluble polymer, a BNNT, and a library molecule with an affinity for a BNNT of <1E−6 M.

A2.7.7.4: A DNA-encoded macrocycle, a medium comprising >80% organic solvent and a soluble polymer, a BNNT, and a library molecule with an affinity for a BNNT of <1E−7 M.

A2.7.7.5: A DNA-encoded macrocycle, a medium comprising >80% organic solvent and a soluble polymer, a BNNT, and a library molecule with an affinity for a BNNT of <1E−8 M.

A2.7.7.6: A DNA-encoded macrocycle, a medium comprising >80% organic solvent and a soluble polymer, a BNNT, and a library molecule with an affinity for a BNNT of <1E−9 M.

A2.7.7.7: A DNA-encoded macrocycle, a medium comprising >80% organic solvent and a soluble polymer, a BNNT, and a library molecule with an affinity for a BNNT of <1E−10 M.

A2.7.7.8: A DNA-encoded macrocycle, a medium comprising >80% organic solvent and a soluble polymer, a BNNT, and a library molecule with an affinity for a BNNT of <1E−12 M.

A2.8.1.1: A DNA-encoded macrocycle, a medium comprising >95% organic solvent, a CNT, and a library molecule with an affinity for a CNT of <1E−2 M.

A2.8.1.2: A DNA-encoded macrocycle, a medium comprising >95% organic solvent, a CNT, and a library molecule with an affinity for a CNT of <1E−4 M.

A2.8.1.3: A DNA-encoded macrocycle, a medium comprising >95% organic solvent, a CNT, and a library molecule with an affinity for a CNT of <1E−6 M.

A2.8.1.4: A DNA-encoded macrocycle, a medium comprising >95% organic solvent, a CNT, and a library molecule with an affinity for a CNT of <1E−7 M.

A2.8.1.5: A DNA-encoded macrocycle, a medium comprising >95% organic solvent, a CNT, and a library molecule with an affinity for a CNT of <1E−8 M.

A2.8.1.6: A DNA-encoded macrocycle, a medium comprising >95% organic solvent, a CNT, and a library molecule with an affinity for a CNT of <1E−9 M.

A2.8.1.7: A DNA-encoded macrocycle, a medium comprising >95% organic solvent, a CNT, and a library molecule with an affinity for a CNT of <1E−10 M.

A2.8.1.8: A DNA-encoded macrocycle, a medium comprising >95% organic solvent, a CNT, and a library molecule with an affinity for a CNT of <1E−12 M.

A2.8.2.1: A DNA-encoded macrocycle, a medium comprising >95% organic solvent, a GS, and a library molecule with an affinity for a GS of <1E−2 M.

A2.8.2.2: A DNA-encoded macrocycle, a medium comprising >95% organic solvent, a GS, and a library molecule with an affinity for a GS of <1E−4 M.

A2.8.2.3: A DNA-encoded macrocycle, a medium comprising >95% organic solvent, a GS, and a library molecule with an affinity for a GS of <1E−6 M.

A2.8.2.4: A DNA-encoded macrocycle, a medium comprising >95% organic solvent, a GS, and a library molecule with an affinity for a GS of <1E−7 M.

A2.8.2.5: A DNA-encoded macrocycle, a medium comprising >95% organic solvent, a GS, and a library molecule with an affinity for a GS of <1E−8 M.

A2.8.2.6: A DNA-encoded macrocycle, a medium comprising >95% organic solvent, a GS, and a library molecule with an affinity for a GS of <1E−9 M.

A2.8.2.7: A DNA-encoded macrocycle, a medium comprising >95% organic solvent, a GS, and a library molecule with an affinity for a GS of <1E−10 M.

A2.8.2.8: A DNA-encoded macrocycle, a medium comprising >95% organic solvent, a GS, and a library molecule with an affinity for a GS of <1E−12 M.

A2.8.3.1: A DNA-encoded macrocycle, a medium comprising >95% organic solvent, a SWNT, and a library molecule with an affinity for a SWNT of <1E−2 M.

A2.8.3.2: A DNA-encoded macrocycle, a medium comprising >95% organic solvent, a SWNT, and a library molecule with an affinity for a SWNT of <1E−4 M.

A2.8.3.3: A DNA-encoded macrocycle, a medium comprising >95% organic solvent, a SWNT, and a library molecule with an affinity for a SWNT of <1E−6 M.

A2.8.3.4: A DNA-encoded macrocycle, a medium comprising >95% organic solvent, a SWNT, and a library molecule with an affinity for a SWNT of <1E−7 M.

A2.8.3.5: A DNA-encoded macrocycle, a medium comprising >95% organic solvent, a SWNT, and a library molecule with an affinity for a SWNT of <1E−8 M.

A2.8.3.6: A DNA-encoded macrocycle, a medium comprising >95% organic solvent, a SWNT, and a library molecule with an affinity for a SWNT of <1E−9 M.

A2.8.3.7: A DNA-encoded macrocycle, a medium comprising >95% organic solvent, a SWNT, and a library molecule with an affinity for a SWNT of <1E−10 M.

A2.8.3.8: A DNA-encoded macrocycle, a medium comprising >95% organic solvent, a SWNT, and a library molecule with an affinity for a SWNT of <1E−12 M.

A2.8.4.1: A DNA-encoded macrocycle, a medium comprising >95% organic solvent, a MWNT, and a library molecule with an affinity for a MWNT of <1E−2 M.

A2.8.4.2: A DNA-encoded macrocycle, a medium comprising >95% organic solvent, a MWNT, and a library molecule with an affinity for a MWNT of <1E−4 M.

A2.8.4.3: A DNA-encoded macrocycle, a medium comprising >95% organic solvent, a MWNT, and a library molecule with an affinity for a MWNT of <1E−6 M.

A2.8.4.4: A DNA-encoded macrocycle, a medium comprising >95% organic solvent, a MWNT, and a library molecule with an affinity for a MWNT of <1E−7 M.

A2.8.4.5: A DNA-encoded macrocycle, a medium comprising >95% organic solvent, a MWNT, and a library molecule with an affinity for a MWNT of <1E−8 M.

A2.8.4.6: A DNA-encoded macrocycle, a medium comprising >95% organic solvent, a MWNT, and a library molecule with an affinity for a MWNT of <1E−9 M.

A2.8.4.7: A DNA-encoded macrocycle, a medium comprising >95% organic solvent, a MWNT, and a library molecule with an affinity for a MWNT of <1E−10 M.

A2.8.4.8: A DNA-encoded macrocycle, a medium comprising >95% organic solvent, a MWNT, and a library molecule with an affinity for a MWNT of <1E−12 M.

A2.8.5.1: A DNA-encoded macrocycle, a medium comprising >95% organic solvent, a nanotube, and a library molecule with an affinity for a nanotube of <1E−2 M.

A2.8.5.2: A DNA-encoded macrocycle, a medium comprising >95% organic solvent, a nanotube, and a library molecule with an affinity for a nanotube of <1E−4 M.

A2.8.5.3: A DNA-encoded macrocycle, a medium comprising >95% organic solvent, a nanotube, and a library molecule with an affinity for a nanotube of <1E−6 M.

A2.8.5.4: A DNA-encoded macrocycle, a medium comprising >95% organic solvent, a nanotube, and a library molecule with an affinity for a nanotube of <1E−7 M.

A2.8.5.5: A DNA-encoded macrocycle, a medium comprising >95% organic solvent, a nanotube, and a library molecule with an affinity for a nanotube of <1E−8 M.

A2.8.5.6: A DNA-encoded macrocycle, a medium comprising >95% organic solvent, a nanotube, and a library molecule with an affinity for a nanotube of <1E−9 M.

A2.8.5.7: A DNA-encoded macrocycle, a medium comprising >95% organic solvent, a nanotube, and a library molecule with an affinity for a nanotube of <1E−10 M.

A2.8.5.8: A DNA-encoded macrocycle, a medium comprising >95% organic solvent, a nanotube, and a library molecule with an affinity for a nanotube of <1E−12 M.

A2.8.6.1: A DNA-encoded macrocycle, a medium comprising >95% organic solvent, a fullerene, and a library molecule with an affinity for a fullerene of <1E−2 M.

A2.8.6.2: A DNA-encoded macrocycle, a medium comprising >95% organic solvent, a fullerene, and a library molecule with an affinity for a fullerene of <1E−4 M.

A2.8.6.3: A DNA-encoded macrocycle, a medium comprising >95% organic solvent, a fullerene, and a library molecule with an affinity for a fullerene of <1E−6 M.

A2.8.6.4: A DNA-encoded macrocycle, a medium comprising >95% organic solvent, a fullerene, and a library molecule with an affinity for a fullerene of <1E−7 M.

A2.8.6.5: A DNA-encoded macrocycle, a medium comprising >95% organic solvent, a fullerene, and a library molecule with an affinity for a fullerene of <1E−8 M.

A2.8.6.6: A DNA-encoded macrocycle, a medium comprising >95% organic solvent, a fullerene, and a library molecule with an affinity for a fullerene of <1E−9 M.

A2.8.6.7: A DNA-encoded macrocycle, a medium comprising >95% organic solvent, a fullerene, and a library molecule with an affinity for a fullerene of <1E−10 M.

A2.8.6.8: A DNA-encoded macrocycle, a medium comprising >95% organic solvent, a fullerene, and a library molecule with an affinity for a fullerene of <1E−12 M.

A2.8.7.1: A DNA-encoded macrocycle, a medium comprising >95% organic solvent, a BNNT, and a library molecule with an affinity for a BNNT of <1E−2 M.

A2.8.7.2: A DNA-encoded macrocycle, a medium comprising >95% organic solvent, a BNNT, and a library molecule with an affinity for a BNNT of <1E−4 M.

A2.8.7.3: A DNA-encoded macrocycle, a medium comprising >95% organic solvent, a BNNT, and a library molecule with an affinity for a BNNT of <1E−6 M.

A2.8.7.4: A DNA-encoded macrocycle, a medium comprising >95% organic solvent, a BNNT, and a library molecule with an affinity for a BNNT of <1E−7 M.

A2.8.7.5: A DNA-encoded macrocycle, a medium comprising >95% organic solvent, a BNNT, and a library molecule with an affinity for a BNNT of <1E−8 M.

A2.8.7.6: A DNA-encoded macrocycle, a medium comprising >95% organic solvent, a BNNT, and a library molecule with an affinity for a BNNT of <1E−9 M.

A2.8.7.7: A DNA-encoded macrocycle, a medium comprising >95% organic solvent, a BNNT, and a library molecule with an affinity for a BNNT of <1E−10 M.

A2.8.7.8: A DNA-encoded macrocycle, a medium comprising >95% organic solvent, a BNNT, and a library molecule with an affinity for a BNNT of <1E−12 M.

A2.9.1.1: A DNA-encoded macrocycle, a medium comprising >95% organic solvent and a soluble polymer, a CNT, and a library molecule with an affinity for a CNT of <1E−2 M.

A2.9.1.2: A DNA-encoded macrocycle, a medium comprising >95% organic solvent and a soluble polymer, a CNT, and a library molecule with an affinity for a CNT of <1E−4 M.

A2.9.1.3: A DNA-encoded macrocycle, a medium comprising >95% organic solvent and a soluble polymer, a CNT, and a library molecule with an affinity for a CNT of <1E−6 M.

A2.9.1.4: A DNA-encoded macrocycle, a medium comprising >95% organic solvent and a soluble polymer, a CNT, and a library molecule with an affinity for a CNT of <1E−7 M.

A2.9.1.5: A DNA-encoded macrocycle, a medium comprising >95% organic solvent and a soluble polymer, a CNT, and a library molecule with an affinity for a CNT of <1E−8 M.

A2.9.1.6: A DNA-encoded macrocycle, a medium comprising >95% organic solvent and a soluble polymer, a CNT, and a library molecule with an affinity for a CNT of <1E−9 M.

A2.9.1.7: A DNA-encoded macrocycle, a medium comprising >95% organic solvent and a soluble polymer, a CNT, and a library molecule with an affinity for a CNT of <1E−10 M.

A2.9.1.8: A DNA-encoded macrocycle, a medium comprising >95% organic solvent and a soluble polymer, a CNT, and a library molecule with an affinity for a CNT of <1E−12 M.

A2.9.2.1: A DNA-encoded macrocycle, a medium comprising >95% organic solvent and a soluble polymer, a GS, and a library molecule with an affinity for a GS of <1E−2 M.

A2.9.2.2: A DNA-encoded macrocycle, a medium comprising >95% organic solvent and a soluble polymer, a GS, and a library molecule with an affinity for a GS of <1E−4 M.

A2.9.2.3: A DNA-encoded macrocycle, a medium comprising >95% organic solvent and a soluble polymer, a GS, and a library molecule with an affinity for a GS of <1E−6 M.

A2.9.2.4: A DNA-encoded macrocycle, a medium comprising >95% organic solvent and a soluble polymer, a GS, and a library molecule with an affinity for a GS of <1E−7 M.

A2.9.2.5: A DNA-encoded macrocycle, a medium comprising >95% organic solvent and a soluble polymer, a GS, and a library molecule with an affinity for a GS of <1E−8 M.

A2.9.2.6: A DNA-encoded macrocycle, a medium comprising >95% organic solvent and a soluble polymer, a GS, and a library molecule with an affinity for a GS of <1E−9 M.

A2.9.2.7: A DNA-encoded macrocycle, a medium comprising >95% organic solvent and a soluble polymer, a GS, and a library molecule with an affinity for a GS of <1E−10 M.

A2.9.2.8: A DNA-encoded macrocycle, a medium comprising >95% organic solvent and a soluble polymer, a GS, and a library molecule with an affinity for a GS of <1E−12 M.

A2.9.3.1: A DNA-encoded macrocycle, a medium comprising >95% organic solvent and a soluble polymer, a SWNT, and a library molecule with an affinity for a SWNT of <1E−2 M.

A2.9.3.2: A DNA-encoded macrocycle, a medium comprising >95% organic solvent and a soluble polymer, a SWNT, and a library molecule with an affinity for a SWNT of <1E−4 M.

A2.9.3.3: A DNA-encoded macrocycle, a medium comprising >95% organic solvent and a soluble polymer, a SWNT, and a library molecule with an affinity for a SWNT of <1E−6 M.

A2.9.3.4: A DNA-encoded macrocycle, a medium comprising >95% organic solvent and a soluble polymer, a SWNT, and a library molecule with an affinity for a SWNT of <1E−7 M.

A2.9.3.5: A DNA-encoded macrocycle, a medium comprising >95% organic solvent and a soluble polymer, a SWNT, and a library molecule with an affinity for a SWNT of <1E−8 M.

A2.9.3.6: A DNA-encoded macrocycle, a medium comprising >95% organic solvent and a soluble polymer, a SWNT, and a library molecule with an affinity for a SWNT of <1E−9 M.

A2.9.3.7: A DNA-encoded macrocycle, a medium comprising >95% organic solvent and a soluble polymer, a SWNT, and a library molecule with an affinity for a SWNT of <1E−10 M.

A2.9.3.8: A DNA-encoded macrocycle, a medium comprising >95% organic solvent and a soluble polymer, a SWNT, and a library molecule with an affinity for a SWNT of <1E−12 M.

A2.9.4.1: A DNA-encoded macrocycle, a medium comprising >95% organic solvent and a soluble polymer, a MWNT, and a library molecule with an affinity for a MWNT of <1E−2 M.

A2.9.4.2: A DNA-encoded macrocycle, a medium comprising >95% organic solvent and a soluble polymer, a MWNT, and a library molecule with an affinity for a MWNT of <1E−4 M.

A2.9.4.3: A DNA-encoded macrocycle, a medium comprising >95% organic solvent and a soluble polymer, a MWNT, and a library molecule with an affinity for a MWNT of <1E−6 M.

A2.9.4.4: A DNA-encoded macrocycle, a medium comprising >95% organic solvent and a soluble polymer, a MWNT, and a library molecule with an affinity for a MWNT of <1E−7 M.

A2.9.4.5: A DNA-encoded macrocycle, a medium comprising >95% organic solvent and a soluble polymer, a MWNT, and a library molecule with an affinity for a MWNT of <1E−8 M.

A2.9.4.6: A DNA-encoded macrocycle, a medium comprising >95% organic solvent and a soluble polymer, a MWNT, and a library molecule with an affinity for a MWNT of <1E−9 M.

A2.9.4.7: A DNA-encoded macrocycle, a medium comprising >95% organic solvent and a soluble polymer, a MWNT, and a library molecule with an affinity for a MWNT of <1E−10 M.

A2.9.4.8: A DNA-encoded macrocycle, a medium comprising >95% organic solvent and a soluble polymer, a MWNT, and a library molecule with an affinity for a MWNT of <1E−12 M.

A2.9.5.1: A DNA-encoded macrocycle, a medium comprising >95% organic solvent and a soluble polymer, a nanotube, and a library molecule with an affinity for a nanotube of <1E−2 M.

A2.9.5.2: A DNA-encoded macrocycle, a medium comprising >95% organic solvent and a soluble polymer, a nanotube, and a library molecule with an affinity for a nanotube of <1E−4 M.

A2.9.5.3: A DNA-encoded macrocycle, a medium comprising >95% organic solvent and a soluble polymer, a nanotube, and a library molecule with an affinity for a nanotube of <1E−6 M.

A2.9.5.4: A DNA-encoded macrocycle, a medium comprising >95% organic solvent and a soluble polymer, a nanotube, and a library molecule with an affinity for a nanotube of <1E−7 M.

A2.9.5.5: A DNA-encoded macrocycle, a medium comprising >95% organic solvent and a soluble polymer, a nanotube, and a library molecule with an affinity for a nanotube of <1E−8 M.

A2.9.5.6: A DNA-encoded macrocycle, a medium comprising >95% organic solvent and a soluble polymer, a nanotube, and a library molecule with an affinity for a nanotube of <1E−9 M.

A2.9.5.7: A DNA-encoded macrocycle, a medium comprising >95% organic solvent and a soluble polymer, a nanotube, and a library molecule with an affinity for a nanotube of <1E−10 M.

A2.9.5.8: A DNA-encoded macrocycle, a medium comprising >95% organic solvent and a soluble polymer, a nanotube, and a library molecule with an affinity for a nanotube of <1E−12 M.

A2.9.6.1: A DNA-encoded macrocycle, a medium comprising >95% organic solvent and a soluble polymer, a fullerene, and a library molecule with an affinity for a fullerene of <1E−2 M.

A2.9.6.2: A DNA-encoded macrocycle, a medium comprising >95% organic solvent and a soluble polymer, a fullerene, and a library molecule with an affinity for a fullerene of <1E−4 M.

A2.9.6.3: A DNA-encoded macrocycle, a medium comprising >95% organic solvent and a soluble polymer, a fullerene, and a library molecule with an affinity for a fullerene of <1E−6 M.

A2.9.6.4: A DNA-encoded macrocycle, a medium comprising >95% organic solvent and a soluble polymer, a fullerene, and a library molecule with an affinity for a fullerene of <1E−7 M.

A2.9.6.5: A DNA-encoded macrocycle, a medium comprising >95% organic solvent and a soluble polymer, a fullerene, and a library molecule with an affinity for a fullerene of <1E−8 M.

A2.9.6.6: A DNA-encoded macrocycle, a medium comprising >95% organic solvent and a soluble polymer, a fullerene, and a library molecule with an affinity for a fullerene of <1E−9 M.

A2.9.6.7: A DNA-encoded macrocycle, a medium comprising >95% organic solvent and a soluble polymer, a fullerene, and a library molecule with an affinity for a fullerene of <1E−10 M.

A2.9.6.8: A DNA-encoded macrocycle, a medium comprising >95% organic solvent and a soluble polymer, a fullerene, and a library molecule with an affinity for a fullerene of <1E−12 M.

A2.9.7.1: A DNA-encoded macrocycle, a medium comprising >95% organic solvent and a soluble polymer, a BNNT, and a library molecule with an affinity for a BNNT of <1E−2 M.

A2.9.7.2: A DNA-encoded macrocycle, a medium comprising >95% organic solvent and a soluble polymer, a BNNT, and a library molecule with an affinity for a BNNT of <1E−4 M.

A2.9.7.3: A DNA-encoded macrocycle, a medium comprising >95% organic solvent and a soluble polymer, a BNNT, and a library molecule with an affinity for a BNNT of <1E−6 M.

A2.9.7.4: A DNA-encoded macrocycle, a medium comprising >95% organic solvent and a soluble polymer, a BNNT, and a library molecule with an affinity for a BNNT of <1E−7 M.

A2.9.7.5: A DNA-encoded macrocycle, a medium comprising >95% organic solvent and a soluble polymer, a BNNT, and a library molecule with an affinity for a BNNT of <1E−8 M.

A2.9.7.6: A DNA-encoded macrocycle, a medium comprising >95% organic solvent and a soluble polymer, a BNNT, and a library molecule with an affinity for a BNNT of <1E−9 M.

A2.9.7.7: A DNA-encoded macrocycle, a medium comprising >95% organic solvent and a soluble polymer, a BNNT, and a library molecule with an affinity for a BNNT of <1E−10 M.

A2.9.7.8: A DNA-encoded macrocycle, a medium comprising >95% organic solvent and a soluble polymer, a BNNT, and a library molecule with an affinity for a BNNT of <1E−12 M.

A3.1.1.1: A DNA-encoded peptide library, a medium comprising an organic solvent, a CNT, and a library molecule with an affinity for a CNT of <1E−2 M.

A3.1.1.2: A DNA-encoded peptide library, a medium comprising an organic solvent, a CNT, and a library molecule with an affinity for a CNT of <1E−4 M.

A3.1.1.3: A DNA-encoded peptide library, a medium comprising an organic solvent, a CNT, and a library molecule with an affinity for a CNT of <1E−6 M.

A3.1.1.4: A DNA-encoded peptide library, a medium comprising an organic solvent, a CNT, and a library molecule with an affinity for a CNT of <1E−7 M.

A3.1.1.5: A DNA-encoded peptide library, a medium comprising an organic solvent, a CNT, and a library molecule with an affinity for a CNT of <1E−8 M.

A3.1.1.6: A DNA-encoded peptide library, a medium comprising an organic solvent, a CNT, and a library molecule with an affinity for a CNT of <1E−9 M.

A3.1.1.7: A DNA-encoded peptide library, a medium comprising an organic solvent, a CNT, and a library molecule with an affinity for a CNT of <1E−10 M.

A3.1.1.8: A DNA-encoded peptide library, a medium comprising an organic solvent, a CNT, and a library molecule with an affinity for a CNT of <1E−12 M.

A3.1.2.1: A DNA-encoded peptide library, a medium comprising an organic solvent, a GS, and a library molecule with an affinity for a GS of <1E−2 M.

A3.1.2.2: A DNA-encoded peptide library, a medium comprising an organic solvent, a GS, and a library molecule with an affinity for a GS of <1E−4 M.

A3.1.2.3: A DNA-encoded peptide library, a medium comprising an organic solvent, a GS, and a library molecule with an affinity for a GS of <1E−6 M.

A3.1.2.4: A DNA-encoded peptide library, a medium comprising an organic solvent, a GS, and a library molecule with an affinity for a GS of <1E−7 M.

A3.1.2.5: A DNA-encoded peptide library, a medium comprising an organic solvent, a GS, and a library molecule with an affinity for a GS of <1E−8 M.

A3.1.2.6: A DNA-encoded peptide library, a medium comprising an organic solvent, a GS, and a library molecule with an affinity for a GS of <1E−9 M.

A3.1.2.7: A DNA-encoded peptide library, a medium comprising an organic solvent, a GS, and a library molecule with an affinity for a GS of <1E−10 M.

A3.1.2.8: A DNA-encoded peptide library, a medium comprising an organic solvent, a GS, and a library molecule with an affinity for a GS of <1E−12 M.

A3.1.3.1: A DNA-encoded peptide library, a medium comprising an organic solvent, a SWNT, and a library molecule with an affinity for a SWNT of <1E−2 M.

A3.1.3.2: A DNA-encoded peptide library, a medium comprising an organic solvent, a SWNT, and a library molecule with an affinity for a SWNT of <1E−4 M.

A3.1.3.3: A DNA-encoded peptide library, a medium comprising an organic solvent, a SWNT, and a library molecule with an affinity for a SWNT of <1E−6 M.

A3.1.3.4: A DNA-encoded peptide library, a medium comprising an organic solvent, a SWNT, and a library molecule with an affinity for a SWNT of <1E−7 M.

A3.1.3.5: A DNA-encoded peptide library, a medium comprising an organic solvent, a SWNT, and a library molecule with an affinity for a SWNT of <1E−8 M.

A3.1.3.6: A DNA-encoded peptide library, a medium comprising an organic solvent, a SWNT, and a library molecule with an affinity for a SWNT of <1E−9 M.

A3.1.3.7: A DNA-encoded peptide library, a medium comprising an organic solvent, a SWNT, and a library molecule with an affinity for a SWNT of <1E−10 M.

A3.1.3.8: A DNA-encoded peptide library, a medium comprising an organic solvent, a SWNT, and a library molecule with an affinity for a SWNT of <1E−12 M.

A3.1.4.1: A DNA-encoded peptide library, a medium comprising an organic solvent, a MWNT, and a library molecule with an affinity for a MWNT of <1E−2 M.

A3.1.4.2: A DNA-encoded peptide library, a medium comprising an organic solvent, a MWNT, and a library molecule with an affinity for a MWNT of <1E−4 M.

A3.1.4.3: A DNA-encoded peptide library, a medium comprising an organic solvent, a MWNT, and a library molecule with an affinity for a MWNT of <1E−6 M.

A3.1.4.4: A DNA-encoded peptide library, a medium comprising an organic solvent, a MWNT, and a library molecule with an affinity for a MWNT of <1E−7 M.

A3.1.4.5: A DNA-encoded peptide library, a medium comprising an organic solvent, a MWNT, and a library molecule with an affinity for a MWNT of <1E−8 M.

A3.1.4.6: A DNA-encoded peptide library, a medium comprising an organic solvent, a MWNT, and a library molecule with an affinity for a MWNT of <1E−9 M.

A3.1.4.7: A DNA-encoded peptide library, a medium comprising an organic solvent, a MWNT, and a library molecule with an affinity for a MWNT of <1E−10 M.

A3.1.4.8: A DNA-encoded peptide library, a medium comprising an organic solvent, a MWNT, and a library molecule with an affinity for a MWNT of <1E−12 M.

A3.1.5.1: A DNA-encoded peptide library, a medium comprising an organic solvent, a nanotube, and a library molecule with an affinity for a nanotube of <1E−2 M.

A3.1.5.2: A DNA-encoded peptide library, a medium comprising an organic solvent, a nanotube, and a library molecule with an affinity for a nanotube of <1E−4 M.

A3.1.5.3: A DNA-encoded peptide library, a medium comprising an organic solvent, a nanotube, and a library molecule with an affinity for a nanotube of <1E−6 M.

A3.1.5.4: A DNA-encoded peptide library, a medium comprising an organic solvent, a nanotube, and a library molecule with an affinity for a nanotube of <1E−7 M.

A3.1.5.5: A DNA-encoded peptide library, a medium comprising an organic solvent, a nanotube, and a library molecule with an affinity for a nanotube of <1E−8 M.

A3.1.5.6: A DNA-encoded peptide library, a medium comprising an organic solvent, a nanotube, and a library molecule with an affinity for a nanotube of <1E−9 M.

A3.1.5.7: A DNA-encoded peptide library, a medium comprising an organic solvent, a nanotube, and a library molecule with an affinity for a nanotube of <1E−10 M.

A3.1.5.8: A DNA-encoded peptide library, a medium comprising an organic solvent, a nanotube, and a library molecule with an affinity for a nanotube of <1E−12 M.

A3.1.6.1: A DNA-encoded peptide library, a medium comprising an organic solvent, a fullerene, and a library molecule with an affinity for a fullerene of <1E−2 M.

A3.1.6.2: A DNA-encoded peptide library, a medium comprising an organic solvent, a fullerene, and a library molecule with an affinity for a fullerene of <1E−4 M.

A3.1.6.3: A DNA-encoded peptide library, a medium comprising an organic solvent, a fullerene, and a library molecule with an affinity for a fullerene of <1E−6 M.

A3.1.6.4: A DNA-encoded peptide library, a medium comprising an organic solvent, a fullerene, and a library molecule with an affinity for a fullerene of <1E−7 M.

A3.1.6.5: A DNA-encoded peptide library, a medium comprising an organic solvent, a fullerene, and a library molecule with an affinity for a fullerene of <1E−8 M.

A3.1.6.6: A DNA-encoded peptide library, a medium comprising an organic solvent, a fullerene, and a library molecule with an affinity for a fullerene of <1E−9 M.

A3.1.6.7: A DNA-encoded peptide library, a medium comprising an organic solvent, a fullerene, and a library molecule with an affinity for a fullerene of <1E−10 M.

A3.1.6.8: A DNA-encoded peptide library, a medium comprising an organic solvent, a fullerene, and a library molecule with an affinity for a fullerene of <1E−12 M.

A3.1.7.1: A DNA-encoded peptide library, a medium comprising an organic solvent, a BNNT, and a library molecule with an affinity for a BNNT of <1E−2 M.

A3.1.7.2: A DNA-encoded peptide library, a medium comprising an organic solvent, a BNNT, and a library molecule with an affinity for a BNNT of <1E−4 M.

A3.1.7.3: A DNA-encoded peptide library, a medium comprising an organic solvent, a BNNT, and a library molecule with an affinity for a BNNT of <1E−6 M.

A3.1.7.4: A DNA-encoded peptide library, a medium comprising an organic solvent, a BNNT, and a library molecule with an affinity for a BNNT of <1E−7 M.

A3.1.7.5: A DNA-encoded peptide library, a medium comprising an organic solvent, a BNNT, and a library molecule with an affinity for a BNNT of <1E−8 M.

A3.1.7.6: A DNA-encoded peptide library, a medium comprising an organic solvent, a BNNT, and a library molecule with an affinity for a BNNT of <1E−9 M.

A3.1.7.7: A DNA-encoded peptide library, a medium comprising an organic solvent, a BNNT, and a library molecule with an affinity for a BNNT of <1E−10 M.

A3.1.7.8: A DNA-encoded peptide library, a medium comprising an organic solvent, a BNNT, and a library molecule with an affinity for a BNNT of <1E−12 M.

A3.2.1.1: A DNA-encoded peptide library, a medium comprising an organic solvent and a soluble polymer, a CNT, and a library molecule with an affinity for a CNT of <1E−2 M.

A3.2.1.2: A DNA-encoded peptide library, a medium comprising an organic solvent and a soluble polymer, a CNT, and a library molecule with an affinity for a CNT of <1E−4 M.

A3.2.1.3: A DNA-encoded peptide library, a medium comprising an organic solvent and a soluble polymer, a CNT, and a library molecule with an affinity for a CNT of <1E−6 M.

A3.2.1.4: A DNA-encoded peptide library, a medium comprising an organic solvent and a soluble polymer, a CNT, and a library molecule with an affinity for a CNT of <1E−7 M.

A3.2.1.5: A DNA-encoded peptide library, a medium comprising an organic solvent and a soluble polymer, a CNT, and a library molecule with an affinity for a CNT of <1E−8 M.

A3.2.1.6: A DNA-encoded peptide library, a medium comprising an organic solvent and a soluble polymer, a CNT, and a library molecule with an affinity for a CNT of <1E−9 M.

A3.2.1.7: A DNA-encoded peptide library, a medium comprising an organic solvent and a soluble polymer, a CNT, and a library molecule with an affinity for a CNT of <1E−10 M.

A3.2.1.8: A DNA-encoded peptide library, a medium comprising an organic solvent and a soluble polymer, a CNT, and a library molecule with an affinity for a CNT of <1E−12 M.

A3.2.2.1: A DNA-encoded peptide library, a medium comprising an organic solvent and a soluble polymer, a GS, and a library molecule with an affinity for a GS of <1E−2 M.

A3.2.2.2: A DNA-encoded peptide library, a medium comprising an organic solvent and a soluble polymer, a GS, and a library molecule with an affinity for a GS of <1E−4 M.

A3.2.2.3: A DNA-encoded peptide library, a medium comprising an organic solvent and a soluble polymer, a GS, and a library molecule with an affinity for a GS of <1E−6 M.

A3.2.2.4: A DNA-encoded peptide library, a medium comprising an organic solvent and a soluble polymer, a GS, and a library molecule with an affinity for a GS of <1E−7 M.

A3.2.2.5: A DNA-encoded peptide library, a medium comprising an organic solvent and a soluble polymer, a GS, and a library molecule with an affinity for a GS of <1E−8 M.

A3.2.2.6: A DNA-encoded peptide library, a medium comprising an organic solvent and a soluble polymer, a GS, and a library molecule with an affinity for a GS of <1E−9 M.

A3.2.2.7: A DNA-encoded peptide library, a medium comprising an organic solvent and a soluble polymer, a GS, and a library molecule with an affinity for a GS of <1E−10 M.

A3.2.2.8: A DNA-encoded peptide library, a medium comprising an organic solvent and a soluble polymer, a GS, and a library molecule with an affinity for a GS of <1E−12 M.

A3.2.3.1: A DNA-encoded peptide library, a medium comprising an organic solvent and a soluble polymer, a SWNT, and a library molecule with an affinity for a SWNT of <1E−2 M.

A3.2.3.2: A DNA-encoded peptide library, a medium comprising an organic solvent and a soluble polymer, a SWNT, and a library molecule with an affinity for a SWNT of <1E−4 M.

A3.2.3.3: A DNA-encoded peptide library, a medium comprising an organic solvent and a soluble polymer, a SWNT, and a library molecule with an affinity for a SWNT of <1E−6 M.

A3.2.3.4: A DNA-encoded peptide library, a medium comprising an organic solvent and a soluble polymer, a SWNT, and a library molecule with an affinity for a SWNT of <1E−7 M.

A3.2.3.5: A DNA-encoded peptide library, a medium comprising an organic solvent and a soluble polymer, a SWNT, and a library molecule with an affinity for a SWNT of <1E−8 M.

A3.2.3.6: A DNA-encoded peptide library, a medium comprising an organic solvent and a soluble polymer, a SWNT, and a library molecule with an affinity for a SWNT of <1E−9 M.

A3.2.3.7: A DNA-encoded peptide library, a medium comprising an organic solvent and a soluble polymer, a SWNT, and a library molecule with an affinity for a SWNT of <1E−10 M.

A3.2.3.8: A DNA-encoded peptide library, a medium comprising an organic solvent and a soluble polymer, a SWNT, and a library molecule with an affinity for a SWNT of <1E−12 M.

A3.2.4.1: A DNA-encoded peptide library, a medium comprising an organic solvent and a soluble polymer, a MWNT, and a library molecule with an affinity for a MWNT of <1E−2 M.

A3.2.4.2: A DNA-encoded peptide library, a medium comprising an organic solvent and a soluble polymer, a MWNT, and a library molecule with an affinity for a MWNT of <1E−4 M.

A3.2.4.3: A DNA-encoded peptide library, a medium comprising an organic solvent and a soluble polymer, a MWNT, and a library molecule with an affinity for a MWNT of <1E−6 M.

A3.2.4.4: A DNA-encoded peptide library, a medium comprising an organic solvent and a soluble polymer, a MWNT, and a library molecule with an affinity for a MWNT of <1E−7 M.

A3.2.4.5: A DNA-encoded peptide library, a medium comprising an organic solvent and a soluble polymer, a MWNT, and a library molecule with an affinity for a MWNT of <1E−8 M.

A3.2.4.6: A DNA-encoded peptide library, a medium comprising an organic solvent and a soluble polymer, a MWNT, and a library molecule with an affinity for a MWNT of <1E−9 M.

A3.2.4.7: A DNA-encoded peptide library, a medium comprising an organic solvent and a soluble polymer, a MWNT, and a library molecule with an affinity for a MWNT of <1E−10 M.

A3.2.4.8: A DNA-encoded peptide library, a medium comprising an organic solvent and a soluble polymer, a MWNT, and a library molecule with an affinity for a MWNT of <1E−12 M.

A3.2.5.1: A DNA-encoded peptide library, a medium comprising an organic solvent and a soluble polymer, a nanotube, and a library molecule with an affinity for a nanotube of <1E−2 M.

A3.2.5.2: A DNA-encoded peptide library, a medium comprising an organic solvent and a soluble polymer, a nanotube, and a library molecule with an affinity for a nanotube of <1E−4 M.

A3.2.5.3: A DNA-encoded peptide library, a medium comprising an organic solvent and a soluble polymer, a nanotube, and a library molecule with an affinity for a nanotube of <1E−6 M.

A3.2.5.4: A DNA-encoded peptide library, a medium comprising an organic solvent and a soluble polymer, a nanotube, and a library molecule with an affinity for a nanotube of <1E−7 M.

A3.2.5.5: A DNA-encoded peptide library, a medium comprising an organic solvent and a soluble polymer, a nanotube, and a library molecule with an affinity for a nanotube of <1E−8 M.

A3.2.5.6: A DNA-encoded peptide library, a medium comprising an organic solvent and a soluble polymer, a nanotube, and a library molecule with an affinity for a nanotube of <1E−9 M.

A3.2.5.7: A DNA-encoded peptide library, a medium comprising an organic solvent and a soluble polymer, a nanotube, and a library molecule with an affinity for a nanotube of <1E−10 M.

A3.2.5.8: A DNA-encoded peptide library, a medium comprising an organic solvent and a soluble polymer, a nanotube, and a library molecule with an affinity for a nanotube of <1E−12 M.

A3.2.6.1: A DNA-encoded peptide library, a medium comprising an organic solvent and a soluble polymer, a fullerene, and a library molecule with an affinity for a fullerene of <1E−2 M.

A3.2.6.2: A DNA-encoded peptide library, a medium comprising an organic solvent and a soluble polymer, a fullerene, and a library molecule with an affinity for a fullerene of <1E−4 M.

A3.2.6.3: A DNA-encoded peptide library, a medium comprising an organic solvent and a soluble polymer, a fullerene, and a library molecule with an affinity for a fullerene of <1E−6 M.

A3.2.6.4: A DNA-encoded peptide library, a medium comprising an organic solvent and a soluble polymer, a fullerene, and a library molecule with an affinity for a fullerene of <1E−7 M.

A3.2.6.5: A DNA-encoded peptide library, a medium comprising an organic solvent and a soluble polymer, a fullerene, and a library molecule with an affinity for a fullerene of <1E−8 M.

A3.2.6.6: A DNA-encoded peptide library, a medium comprising an organic solvent and a soluble polymer, a fullerene, and a library molecule with an affinity for a fullerene of <1E−9 M.

A3.2.6.7: A DNA-encoded peptide library, a medium comprising an organic solvent and a soluble polymer, a fullerene, and a library molecule with an affinity for a fullerene of <1E−10 M.

A3.2.6.8: A DNA-encoded peptide library, a medium comprising an organic solvent and a soluble polymer, a fullerene, and a library molecule with an affinity for a fullerene of <1E−12 M.

A3.2.7.1: A DNA-encoded peptide library, a medium comprising an organic solvent and a soluble polymer, a BNNT, and a library molecule with an affinity for a BNNT of <1E−2 M.

A3.2.7.2: A DNA-encoded peptide library, a medium comprising an organic solvent and a soluble polymer, a BNNT, and a library molecule with an affinity for a BNNT of <1E−4 M.

A3.2.7.3: A DNA-encoded peptide library, a medium comprising an organic solvent and a soluble polymer, a BNNT, and a library molecule with an affinity for a BNNT of <1E−6 M.

A3.2.7.4: A DNA-encoded peptide library, a medium comprising an organic solvent and a soluble polymer, a BNNT, and a library molecule with an affinity for a BNNT of <1E−7 M.

A3.2.7.5: A DNA-encoded peptide library, a medium comprising an organic solvent and a soluble polymer, a BNNT, and a library molecule with an affinity for a BNNT of <1E−8 M.

A3.2.7.6: A DNA-encoded peptide library, a medium comprising an organic solvent and a soluble polymer, a BNNT, and a library molecule with an affinity for a BNNT of <1E−9 M.

A3.2.7.7: A DNA-encoded peptide library, a medium comprising an organic solvent and a soluble polymer, a BNNT, and a library molecule with an affinity for a BNNT of <1E−10 M.

A3.2.7.8: A DNA-encoded peptide library, a medium comprising an organic solvent and a soluble polymer, a BNNT, and a library molecule with an affinity for a BNNT of <1E−12 M.

A3.3.1.1: A DNA-encoded peptide library, a medium comprising a soluble polymer, a CNT, and a library molecule with an affinity for a CNT of <1E−2 M.

A3.3.1.2: A DNA-encoded peptide library, a medium comprising a soluble polymer, a CNT, and a library molecule with an affinity for a CNT of <1E−4 M.

A3.3.1.3: A DNA-encoded peptide library, a medium comprising a soluble polymer, a CNT, and a library molecule with an affinity for a CNT of <1E−6 M.

A3.3.1.4: A DNA-encoded peptide library, a medium comprising a soluble polymer, a CNT, and a library molecule with an affinity for a CNT of <1E−7 M.

A3.3.1.5: A DNA-encoded peptide library, a medium comprising a soluble polymer, a CNT, and a library molecule with an affinity for a CNT of <1E−8 M.

A3.3.1.6: A DNA-encoded peptide library, a medium comprising a soluble polymer, a CNT, and a library molecule with an affinity for a CNT of <1E−9 M.

A3.3.1.7: A DNA-encoded peptide library, a medium comprising a soluble polymer, a CNT, and a library molecule with an affinity for a CNT of <1E−10 M.

A3.3.1.8: A DNA-encoded peptide library, a medium comprising a soluble polymer, a CNT, and a library molecule with an affinity for a CNT of <1E−12 M.

A3.3.2.1: A DNA-encoded peptide library, a medium comprising a soluble polymer, a GS, and a library molecule with an affinity for a GS of <1E−2 M.

A3.3.2.2: A DNA-encoded peptide library, a medium comprising a soluble polymer, a GS, and a library molecule with an affinity for a GS of <1E−4 M.

A3.3.2.3: A DNA-encoded peptide library, a medium comprising a soluble polymer, a GS, and a library molecule with an affinity for a GS of <1E−6 M.

A3.3.2.4: A DNA-encoded peptide library, a medium comprising a soluble polymer, a GS, and a library molecule with an affinity for a GS of <1E−7 M.

A3.3.2.5: A DNA-encoded peptide library, a medium comprising a soluble polymer, a GS, and a library molecule with an affinity for a GS of <1E−8 M.

A3.3.2.6: A DNA-encoded peptide library, a medium comprising a soluble polymer, a GS, and a library molecule with an affinity for a GS of <1E−9 M.

A3.3.2.7: A DNA-encoded peptide library, a medium comprising a soluble polymer, a GS, and a library molecule with an affinity for a GS of <1E−10 M.

A3.3.2.8: A DNA-encoded peptide library, a medium comprising a soluble polymer, a GS, and a library molecule with an affinity for a GS of <1E−12 M.

A3.3.3.1: A DNA-encoded peptide library, a medium comprising a soluble polymer, a SWNT, and a library molecule with an affinity for a SWNT of <1E−2 M.

A3.3.3.2: A DNA-encoded peptide library, a medium comprising a soluble polymer, a SWNT, and a library molecule with an affinity for a SWNT of <1E−4 M.

A3.3.3.3: A DNA-encoded peptide library, a medium comprising a soluble polymer, a SWNT, and a library molecule with an affinity for a SWNT of <1E−6 M.

A3.3.3.4: A DNA-encoded peptide library, a medium comprising a soluble polymer, a SWNT, and a library molecule with an affinity for a SWNT of <1E−7 M.

A3.3.3.5: A DNA-encoded peptide library, a medium comprising a soluble polymer, a SWNT, and a library molecule with an affinity for a SWNT of <1E−8 M.

A3.3.3.6: A DNA-encoded peptide library, a medium comprising a soluble polymer, a SWNT, and a library molecule with an affinity for a SWNT of <1E−9 M.

A3.3.3.7: A DNA-encoded peptide library, a medium comprising a soluble polymer, a SWNT, and a library molecule with an affinity for a SWNT of <1E−10 M.

A3.3.3.8: A DNA-encoded peptide library, a medium comprising a soluble polymer, a SWNT, and a library molecule with an affinity for a SWNT of <1E−12 M.

A3.3.4.1: A DNA-encoded peptide library, a medium comprising a soluble polymer, a MWNT, and a library molecule with an affinity for a MWNT of <1E−2 M.

A3.3.4.2: A DNA-encoded peptide library, a medium comprising a soluble polymer, a MWNT, and a library molecule with an affinity for a MWNT of <1E−4 M.

A3.3.4.3: A DNA-encoded peptide library, a medium comprising a soluble polymer, a MWNT, and a library molecule with an affinity for a MWNT of <1E−6 M.

A3.3.4.4: A DNA-encoded peptide library, a medium comprising a soluble polymer, a MWNT, and a library molecule with an affinity for a MWNT of <1E−7 M.

A3.3.4.5: A DNA-encoded peptide library, a medium comprising a soluble polymer, a MWNT, and a library molecule with an affinity for a MWNT of <1E−8 M.

A3.3.4.6: A DNA-encoded peptide library, a medium comprising a soluble polymer, a MWNT, and a library molecule with an affinity for a MWNT of <1E−9 M.

A3.3.4.7: A DNA-encoded peptide library, a medium comprising a soluble polymer, a MWNT, and a library molecule with an affinity for a MWNT of <1E−10 M.

A3.3.4.8: A DNA-encoded peptide library, a medium comprising a soluble polymer, a MWNT, and a library molecule with an affinity for a MWNT of <1E−12 M.

A3.3.5.1: A DNA-encoded peptide library, a medium comprising a soluble polymer, a nanotube, and a library molecule with an affinity for a nanotube of <1E−2 M.

A3.3.5.2: A DNA-encoded peptide library, a medium comprising a soluble polymer, a nanotube, and a library molecule with an affinity for a nanotube of <1E−4 M.

A3.3.5.3: A DNA-encoded peptide library, a medium comprising a soluble polymer, a nanotube, and a library molecule with an affinity for a nanotube of <1E−6 M.

A3.3.5.4: A DNA-encoded peptide library, a medium comprising a soluble polymer, a nanotube, and a library molecule with an affinity for a nanotube of <1E−7 M.

A3.3.5.5: A DNA-encoded peptide library, a medium comprising a soluble polymer, a nanotube, and a library molecule with an affinity for a nanotube of <1E−8 M.

A3.3.5.6: A DNA-encoded peptide library, a medium comprising a soluble polymer, a nanotube, and a library molecule with an affinity for a nanotube of <1E−9 M.

A3.3.5.7: A DNA-encoded peptide library, a medium comprising a soluble polymer, a nanotube, and a library molecule with an affinity for a nanotube of <1E−10 M.

A3.3.5.8: A DNA-encoded peptide library, a medium comprising a soluble polymer, a nanotube, and a library molecule with an affinity for a nanotube of <1E−12 M.

A3.3.6.1: A DNA-encoded peptide library, a medium comprising a soluble polymer, a fullerene, and a library molecule with an affinity for a fullerene of <1E−2 M.

A3.3.6.2: A DNA-encoded peptide library, a medium comprising a soluble polymer, a fullerene, and a library molecule with an affinity for a fullerene of <1E−4 M.

A3.3.6.3: A DNA-encoded peptide library, a medium comprising a soluble polymer, a fullerene, and a library molecule with an affinity for a fullerene of <1E−6 M.

A3.3.6.4: A DNA-encoded peptide library, a medium comprising a soluble polymer, a fullerene, and a library molecule with an affinity for a fullerene of <1E−7 M.

A3.3.6.5: A DNA-encoded peptide library, a medium comprising a soluble polymer, a fullerene, and a library molecule with an affinity for a fullerene of <1E−8 M.

A3.3.6.6: A DNA-encoded peptide library, a medium comprising a soluble polymer, a fullerene, and a library molecule with an affinity for a fullerene of <1E−9 M.

A3.3.6.7: A DNA-encoded peptide library, a medium comprising a soluble polymer, a fullerene, and a library molecule with an affinity for a fullerene of <1E−10 M.

A3.3.6.8: A DNA-encoded peptide library, a medium comprising a soluble polymer, a fullerene, and a library molecule with an affinity for a fullerene of <1E−12 M.

A3.3.7.1: A DNA-encoded peptide library, a medium comprising a soluble polymer, a BNNT, and a library molecule with an affinity for a BNNT of <1E−2 M.

A3.3.7.2: A DNA-encoded peptide library, a medium comprising a soluble polymer, a BNNT, and a library molecule with an affinity for a BNNT of <1E−4 M.

A3.3.7.3: A DNA-encoded peptide library, a medium comprising a soluble polymer, a BNNT, and a library molecule with an affinity for a BNNT of <1E−6 M.

A3.3.7.4: A DNA-encoded peptide library, a medium comprising a soluble polymer, a BNNT, and a library molecule with an affinity for a BNNT of <1E−7 M.

A3.3.7.5: A DNA-encoded peptide library, a medium comprising a soluble polymer, a BNNT, and a library molecule with an affinity for a BNNT of <1E−8 M.

A3.3.7.6: A DNA-encoded peptide library, a medium comprising a soluble polymer, a BNNT, and a library molecule with an affinity for a BNNT of <1E−9 M.

A3.3.7.7: A DNA-encoded peptide library, a medium comprising a soluble polymer, a BNNT, and a library molecule with an affinity for a BNNT of <1E−10 M.

A3.3.7.8: A DNA-encoded peptide library, a medium comprising a soluble polymer, a BNNT, and a library molecule with an affinity for a BNNT of <1E−12 M.

A3.4.1.1: A DNA-encoded peptide library, a medium comprising >50% organic solvent, a CNT, and a library molecule with an affinity for a CNT of <1E−2 M.

A3.4.1.2: A DNA-encoded peptide library, a medium comprising >50% organic solvent, a CNT, and a library molecule with an affinity for a CNT of <1E−4 M.

A3.4.1.3: A DNA-encoded peptide library, a medium comprising >50% organic solvent, a CNT, and a library molecule with an affinity for a CNT of <1E−6 M.

A3.4.1.4: A DNA-encoded peptide library, a medium comprising >50% organic solvent, a CNT, and a library molecule with an affinity for a CNT of <1E−7 M.

A3.4.1.5: A DNA-encoded peptide library, a medium comprising >50% organic solvent, a CNT, and a library molecule with an affinity for a CNT of <1E−8 M.

A3.4.1.6: A DNA-encoded peptide library, a medium comprising >50% organic solvent, a CNT, and a library molecule with an affinity for a CNT of <1E−9 M.

A3.4.1.7: A DNA-encoded peptide library, a medium comprising >50% organic solvent, a CNT, and a library molecule with an affinity for a CNT of <1E−10 M.

A3.4.1.8: A DNA-encoded peptide library, a medium comprising >50% organic solvent, a CNT, and a library molecule with an affinity for a CNT of <1E−12 M.

A3.4.2.1: A DNA-encoded peptide library, a medium comprising >50% organic solvent, a GS, and a library molecule with an affinity for a GS of <1E−2 M.

A3.4.2.2: A DNA-encoded peptide library, a medium comprising >50% organic solvent, a GS, and a library molecule with an affinity for a GS of <1E−4 M.

A3.4.2.3: A DNA-encoded peptide library, a medium comprising >50% organic solvent, a GS, and a library molecule with an affinity for a GS of <1E−6 M.

A3.4.2.4: A DNA-encoded peptide library, a medium comprising >50% organic solvent, a GS, and a library molecule with an affinity for a GS of <1E−7 M.

A3.4.2.5: A DNA-encoded peptide library, a medium comprising >50% organic solvent, a GS, and a library molecule with an affinity for a GS of <1E−8 M.

A3.4.2.6: A DNA-encoded peptide library, a medium comprising >50% organic solvent, a GS, and a library molecule with an affinity for a GS of <1E−9 M.

A3.4.2.7: A DNA-encoded peptide library, a medium comprising >50% organic solvent, a GS, and a library molecule with an affinity for a GS of <1E−10 M.

A3.4.2.8: A DNA-encoded peptide library, a medium comprising >50% organic solvent, a GS, and a library molecule with an affinity for a GS of <1E−12 M.

A3.4.3.1: A DNA-encoded peptide library, a medium comprising >50% organic solvent, a SWNT, and a library molecule with an affinity for a SWNT of <1E−2 M.

A3.4.3.2: A DNA-encoded peptide library, a medium comprising >50% organic solvent, a SWNT, and a library molecule with an affinity for a SWNT of <1E−4 M.

A3.4.3.3: A DNA-encoded peptide library, a medium comprising >50% organic solvent, a SWNT, and a library molecule with an affinity for a SWNT of <1E−6 M.

A3.4.3.4: A DNA-encoded peptide library, a medium comprising >50% organic solvent, a SWNT, and a library molecule with an affinity for a SWNT of <1E−7 M.

A3.4.3.5: A DNA-encoded peptide library, a medium comprising >50% organic solvent, a SWNT, and a library molecule with an affinity for a SWNT of <1E−8 M.

A3.4.3.6: A DNA-encoded peptide library, a medium comprising >50% organic solvent, a SWNT, and a library molecule with an affinity for a SWNT of <1E−9 M.

A3.4.3.7: A DNA-encoded peptide library, a medium comprising >50% organic solvent, a SWNT, and a library molecule with an affinity for a SWNT of <1E−10 M.

A3.4.3.8: A DNA-encoded peptide library, a medium comprising >50% organic solvent, a SWNT, and a library molecule with an affinity for a SWNT of <1E−12 M.

A3.4.4.1: A DNA-encoded peptide library, a medium comprising >50% organic solvent, a MWNT, and a library molecule with an affinity for a MWNT of <1E−2 M.

A3.4.4.2: A DNA-encoded peptide library, a medium comprising >50% organic solvent, a MWNT, and a library molecule with an affinity for a MWNT of <1E−4 M.

A3.4.4.3: A DNA-encoded peptide library, a medium comprising >50% organic solvent, a MWNT, and a library molecule with an affinity for a MWNT of <1E−6 M.

A3.4.4.4: A DNA-encoded peptide library, a medium comprising >50% organic solvent, a MWNT, and a library molecule with an affinity for a MWNT of <1E−7 M.

A3.4.4.5: A DNA-encoded peptide library, a medium comprising >50% organic solvent, a MWNT, and a library molecule with an affinity for a MWNT of <1E−8 M.

A3.4.4.6: A DNA-encoded peptide library, a medium comprising >50% organic solvent, a MWNT, and a library molecule with an affinity for a MWNT of <1E−9 M.

A3.4.4.7: A DNA-encoded peptide library, a medium comprising >50% organic solvent, a MWNT, and a library molecule with an affinity for a MWNT of <1E−10 M.

A3.4.4.8: A DNA-encoded peptide library, a medium comprising >50% organic solvent, a MWNT, and a library molecule with an affinity for a MWNT of <1E−12 M.

A3.4.5.1: A DNA-encoded peptide library, a medium comprising >50% organic solvent, a nanotube, and a library molecule with an affinity for a nanotube of <1E−2 M.

A3.4.5.2: A DNA-encoded peptide library, a medium comprising >50% organic solvent, a nanotube, and a library molecule with an affinity for a nanotube of <1E−4 M.

A3.4.5.3: A DNA-encoded peptide library, a medium comprising >50% organic solvent, a nanotube, and a library molecule with an affinity for a nanotube of <1E−6 M.

A3.4.5.4: A DNA-encoded peptide library, a medium comprising >50% organic solvent, a nanotube, and a library molecule with an affinity for a nanotube of <1E−7 M.

A3.4.5.5: A DNA-encoded peptide library, a medium comprising >50% organic solvent, a nanotube, and a library molecule with an affinity for a nanotube of <1E−8 M.

A3.4.5.6: A DNA-encoded peptide library, a medium comprising >50% organic solvent, a nanotube, and a library molecule with an affinity for a nanotube of <1E−9 M.

A3.4.5.7: A DNA-encoded peptide library, a medium comprising >50% organic solvent, a nanotube, and a library molecule with an affinity for a nanotube of <1E−10 M.

A3.4.5.8: A DNA-encoded peptide library, a medium comprising >50% organic solvent, a nanotube, and a library molecule with an affinity for a nanotube of <1E−12 M.

A3.4.6.1: A DNA-encoded peptide library, a medium comprising >50% organic solvent, a fullerene, and a library molecule with an affinity for a fullerene of <1E−2 M.

A3.4.6.2: A DNA-encoded peptide library, a medium comprising >50% organic solvent, a fullerene, and a library molecule with an affinity for a fullerene of <1E−4 M.

A3.4.6.3: A DNA-encoded peptide library, a medium comprising >50% organic solvent, a fullerene, and a library molecule with an affinity for a fullerene of <1E−6 M.

A3.4.6.4: A DNA-encoded peptide library, a medium comprising >50% organic solvent, a fullerene, and a library molecule with an affinity for a fullerene of <1E−7 M.

A3.4.6.5: A DNA-encoded peptide library, a medium comprising >50% organic solvent, a fullerene, and a library molecule with an affinity for a fullerene of <1E−8 M.

A3.4.6.6: A DNA-encoded peptide library, a medium comprising >50% organic solvent, a fullerene, and a library molecule with an affinity for a fullerene of <1E−9 M.

A3.4.6.7: A DNA-encoded peptide library, a medium comprising >50% organic solvent, a fullerene, and a library molecule with an affinity for a fullerene of <1E−10 M.

A3.4.6.8: A DNA-encoded peptide library, a medium comprising >50% organic solvent, a fullerene, and a library molecule with an affinity for a fullerene of <1E−12 M.

A3.4.7.1: A DNA-encoded peptide library, a medium comprising >50% organic solvent, a BNNT, and a library molecule with an affinity for a BNNT of <1E−2 M.

A3.4.7.2: A DNA-encoded peptide library, a medium comprising >50% organic solvent, a BNNT, and a library molecule with an affinity for a BNNT of <1E−4 M.

A3.4.7.3: A DNA-encoded peptide library, a medium comprising >50% organic solvent, a BNNT, and a library molecule with an affinity for a BNNT of <1E−6 M.

A3.4.7.4: A DNA-encoded peptide library, a medium comprising >50% organic solvent, a BNNT, and a library molecule with an affinity for a BNNT of <1E−7 M.

A3.4.7.5: A DNA-encoded peptide library, a medium comprising >50% organic solvent, a BNNT, and a library molecule with an affinity for a BNNT of <1E−8 M.

A3.4.7.6: A DNA-encoded peptide library, a medium comprising >50% organic solvent, a BNNT, and a library molecule with an affinity for a BNNT of <1E−9 M.

A3.4.7.7: A DNA-encoded peptide library, a medium comprising >50% organic solvent, a BNNT, and a library molecule with an affinity for a BNNT of <1E−10 M.

A3.4.7.8: A DNA-encoded peptide library, a medium comprising >50% organic solvent, a BNNT, and a library molecule with an affinity for a BNNT of <1E−12 M.

A3.5.1.1: A DNA-encoded peptide library, a medium comprising >50% organic solvent and a soluble polymer, a CNT, and a library molecule with an affinity for a CNT of <1E−2 M.

A3.5.1.2: A DNA-encoded peptide library, a medium comprising >50% organic solvent and a soluble polymer, a CNT, and a library molecule with an affinity for a CNT of <1E−4 M.

A3.5.1.3: A DNA-encoded peptide library, a medium comprising >50% organic solvent and a soluble polymer, a CNT, and a library molecule with an affinity for a CNT of <1E−6 M.

A3.5.1.4: A DNA-encoded peptide library, a medium comprising >50% organic solvent and a soluble polymer, a CNT, and a library molecule with an affinity for a CNT of <1E−7 M.

A3.5.1.5: A DNA-encoded peptide library, a medium comprising >50% organic solvent and a soluble polymer, a CNT, and a library molecule with an affinity for a CNT of <1E−8 M.

A3.5.1.6: A DNA-encoded peptide library, a medium comprising >50% organic solvent and a soluble polymer, a CNT, and a library molecule with an affinity for a CNT of <1E−9 M.

A3.5.1.7: A DNA-encoded peptide library, a medium comprising >50% organic solvent and a soluble polymer, a CNT, and a library molecule with an affinity for a CNT of <1E−10 M.

A3.5.1.8: A DNA-encoded peptide library, a medium comprising >50% organic solvent and a soluble polymer, a CNT, and a library molecule with an affinity for a CNT of <1E−12 M.

A3.5.2.1: A DNA-encoded peptide library, a medium comprising >50% organic solvent and a soluble polymer, a GS, and a library molecule with an affinity for a GS of <1E−2 M.

A3.5.2.2: A DNA-encoded peptide library, a medium comprising >50% organic solvent and a soluble polymer, a GS, and a library molecule with an affinity for a GS of <1E−4 M.

A3.5.2.3: A DNA-encoded peptide library, a medium comprising >50% organic solvent and a soluble polymer, a GS, and a library molecule with an affinity for a GS of <1E−6 M.

A3.5.2.4: A DNA-encoded peptide library, a medium comprising >50% organic solvent and a soluble polymer, a GS, and a library molecule with an affinity for a GS of <1E−7 M.

A3.5.2.5: A DNA-encoded peptide library, a medium comprising >50% organic solvent and a soluble polymer, a GS, and a library molecule with an affinity for a GS of <1E−8 M.

A3.5.2.6: A DNA-encoded peptide library, a medium comprising >50% organic solvent and a soluble polymer, a GS, and a library molecule with an affinity for a GS of <1E−9 M.

A3.5.2.7: A DNA-encoded peptide library, a medium comprising >50% organic solvent and a soluble polymer, a GS, and a library molecule with an affinity for a GS of <1E−10 M.

A3.5.2.8: A DNA-encoded peptide library, a medium comprising >50% organic solvent and a soluble polymer, a GS, and a library molecule with an affinity for a GS of <1E−12 M.

A3.5.3.1: A DNA-encoded peptide library, a medium comprising >50% organic solvent and a soluble polymer, a SWNT, and a library molecule with an affinity for a SWNT of <1E−2 M.

A3.5.3.2: A DNA-encoded peptide library, a medium comprising >50% organic solvent and a soluble polymer, a SWNT, and a library molecule with an affinity for a SWNT of <1E−4 M.

A3.5.3.3: A DNA-encoded peptide library, a medium comprising >50% organic solvent and a soluble polymer, a SWNT, and a library molecule with an affinity for a SWNT of <1E−6 M.

A3.5.3.4: A DNA-encoded peptide library, a medium comprising >50% organic solvent and a soluble polymer, a SWNT, and a library molecule with an affinity for a SWNT of <1E−7 M.

A3.5.3.5: A DNA-encoded peptide library, a medium comprising >50% organic solvent and a soluble polymer, a SWNT, and a library molecule with an affinity for a SWNT of <1E−8 M.

A3.5.3.6: A DNA-encoded peptide library, a medium comprising >50% organic solvent and a soluble polymer, a SWNT, and a library molecule with an affinity for a SWNT of <1E−9 M.

A3.5.3.7: A DNA-encoded peptide library, a medium comprising >50% organic solvent and a soluble polymer, a SWNT, and a library molecule with an affinity for a SWNT of <1E−10 M.

A3.5.3.8: A DNA-encoded peptide library, a medium comprising >50% organic solvent and a soluble polymer, a SWNT, and a library molecule with an affinity for a SWNT of <1E−12 M.

A3.5.4.1: A DNA-encoded peptide library, a medium comprising >50% organic solvent and a soluble polymer, a MWNT, and a library molecule with an affinity for a MWNT of <1E−2 M.

A3.5.4.2: A DNA-encoded peptide library, a medium comprising >50% organic solvent and a soluble polymer, a MWNT, and a library molecule with an affinity for a MWNT of <1E−4 M.

A3.5.4.3: A DNA-encoded peptide library, a medium comprising >50% organic solvent and a soluble polymer, a MWNT, and a library molecule with an affinity for a MWNT of <1E−6 M.

A3.5.4.4: A DNA-encoded peptide library, a medium comprising >50% organic solvent and a soluble polymer, a MWNT, and a library molecule with an affinity for a MWNT of <1E−7 M.

A3.5.4.5: A DNA-encoded peptide library, a medium comprising >50% organic solvent and a soluble polymer, a MWNT, and a library molecule with an affinity for a MWNT of <1E−8 M.

A3.5.4.6: A DNA-encoded peptide library, a medium comprising >50% organic solvent and a soluble polymer, a MWNT, and a library molecule with an affinity for a MWNT of <1E−9 M.

A3.5.4.7: A DNA-encoded peptide library, a medium comprising >50% organic solvent and a soluble polymer, a MWNT, and a library molecule with an affinity for a MWNT of <1E−10 M.

A3.5.4.8: A DNA-encoded peptide library, a medium comprising >50% organic solvent and a soluble polymer, a MWNT, and a library molecule with an affinity for a MWNT of <1E−12 M.

A3.5.5.1: A DNA-encoded peptide library, a medium comprising >50% organic solvent and a soluble polymer, a nanotube, and a library molecule with an affinity for a nanotube of <1E−2 M.

A3.5.5.2: A DNA-encoded peptide library, a medium comprising >50% organic solvent and a soluble polymer, a nanotube, and a library molecule with an affinity for a nanotube of <1E−4 M.

A3.5.5.3: A DNA-encoded peptide library, a medium comprising >50% organic solvent and a soluble polymer, a nanotube, and a library molecule with an affinity for a nanotube of <1E−6 M.

A3.5.5.4: A DNA-encoded peptide library, a medium comprising >50% organic solvent and a soluble polymer, a nanotube, and a library molecule with an affinity for a nanotube of <1E−7 M.

A3.5.5.5: A DNA-encoded peptide library, a medium comprising >50% organic solvent and a soluble polymer, a nanotube, and a library molecule with an affinity for a nanotube of <1E−8 M.

A3.5.5.6: A DNA-encoded peptide library, a medium comprising >50% organic solvent and a soluble polymer, a nanotube, and a library molecule with an affinity for a nanotube of <1E−9 M.

A3.5.5.7: A DNA-encoded peptide library, a medium comprising >50% organic solvent and a soluble polymer, a nanotube, and a library molecule with an affinity for a nanotube of <1E−10 M.

A3.5.5.8: A DNA-encoded peptide library, a medium comprising >50% organic solvent and a soluble polymer, a nanotube, and a library molecule with an affinity for a nanotube of <1E−12 M.

A3.5.6.1: A DNA-encoded peptide library, a medium comprising >50% organic solvent and a soluble polymer, a fullerene, and a library molecule with an affinity for a fullerene of <1E−2 M.

A3.5.6.2: A DNA-encoded peptide library, a medium comprising >50% organic solvent and a soluble polymer, a fullerene, and a library molecule with an affinity for a fullerene of <1E−4 M.

A3.5.6.3: A DNA-encoded peptide library, a medium comprising >50% organic solvent and a soluble polymer, a fullerene, and a library molecule with an affinity for a fullerene of <1E−6 M.

A3.5.6.4: A DNA-encoded peptide library, a medium comprising >50% organic solvent and a soluble polymer, a fullerene, and a library molecule with an affinity for a fullerene of <1E−7 M.

A3.5.6.5: A DNA-encoded peptide library, a medium comprising >50% organic solvent and a soluble polymer, a fullerene, and a library molecule with an affinity for a fullerene of <1E−8 M.

A3.5.6.6: A DNA-encoded peptide library, a medium comprising >50% organic solvent and a soluble polymer, a fullerene, and a library molecule with an affinity for a fullerene of <1E−9 M.

A3.5.6.7: A DNA-encoded peptide library, a medium comprising >50% organic solvent and a soluble polymer, a fullerene, and a library molecule with an affinity for a fullerene of <1E−10 M.

A3.5.6.8: A DNA-encoded peptide library, a medium comprising >50% organic solvent and a soluble polymer, a fullerene, and a library molecule with an affinity for a fullerene of <1E−12 M.

A3.5.7.1: A DNA-encoded peptide library, a medium comprising >50% organic solvent and a soluble polymer, a BNNT, and a library molecule with an affinity for a BNNT of <1E−2 M.

A3.5.7.2: A DNA-encoded peptide library, a medium comprising >50% organic solvent and a soluble polymer, a BNNT, and a library molecule with an affinity for a BNNT of <1E−4 M.

A3.5.7.3: A DNA-encoded peptide library, a medium comprising >50% organic solvent and a soluble polymer, a BNNT, and a library molecule with an affinity for a BNNT of <1E−6 M.

A3.5.7.4: A DNA-encoded peptide library, a medium comprising >50% organic solvent and a soluble polymer, a BNNT, and a library molecule with an affinity for a BNNT of <1E−7 M.

A3.5.7.5: A DNA-encoded peptide library, a medium comprising >50% organic solvent and a soluble polymer, a BNNT, and a library molecule with an affinity for a BNNT of <1E−8 M.

A3.5.7.6: A DNA-encoded peptide library, a medium comprising >50% organic solvent and a soluble polymer, a BNNT, and a library molecule with an affinity for a BNNT of <1E−9 M.

A3.5.7.7: A DNA-encoded peptide library, a medium comprising >50% organic solvent and a soluble polymer, a BNNT, and a library molecule with an affinity for a BNNT of <1E−10 M.

A3.5.7.8: A DNA-encoded peptide library, a medium comprising >50% organic solvent and a soluble polymer, a BNNT, and a library molecule with an affinity for a BNNT of <1E−12 M.

A3.6.1.1: A DNA-encoded peptide library, a medium comprising >80% organic solvent, a CNT, and a library molecule with an affinity for a CNT of <1E−2 M.

A3.6.1.2: A DNA-encoded peptide library, a medium comprising >80% organic solvent, a CNT, and a library molecule with an affinity for a CNT of <1E−4 M.

A3.6.1.3: A DNA-encoded peptide library, a medium comprising >80% organic solvent, a CNT, and a library molecule with an affinity for a CNT of <1E−6 M.

A3.6.1.4: A DNA-encoded peptide library, a medium comprising >80% organic solvent, a CNT, and a library molecule with an affinity for a CNT of <1E−7 M.

A3.6.1.5: A DNA-encoded peptide library, a medium comprising >80% organic solvent, a CNT, and a library molecule with an affinity for a CNT of <1E−8 M.

A3.6.1.6: A DNA-encoded peptide library, a medium comprising >80% organic solvent, a CNT, and a library molecule with an affinity for a CNT of <1E−9 M.

A3.6.1.7: A DNA-encoded peptide library, a medium comprising >80% organic solvent, a CNT, and a library molecule with an affinity for a CNT of <1E−10 M.

A3.6.1.8: A DNA-encoded peptide library, a medium comprising >80% organic solvent, a CNT, and a library molecule with an affinity for a CNT of <1E−12 M.

A3.6.2.1: A DNA-encoded peptide library, a medium comprising >80% organic solvent, a GS, and a library molecule with an affinity for a GS of <1E−2 M.

A3.6.2.2: A DNA-encoded peptide library, a medium comprising >80% organic solvent, a GS, and a library molecule with an affinity for a GS of <1E−4 M.

A3.6.2.3: A DNA-encoded peptide library, a medium comprising >80% organic solvent, a GS, and a library molecule with an affinity for a GS of <1E−6 M.

A3.6.2.4: A DNA-encoded peptide library, a medium comprising >80% organic solvent, a GS, and a library molecule with an affinity for a GS of <1E−7 M.

A3.6.2.5: A DNA-encoded peptide library, a medium comprising >80% organic solvent, a GS, and a library molecule with an affinity for a GS of <1E−8 M.

A3.6.2.6: A DNA-encoded peptide library, a medium comprising >80% organic solvent, a GS, and a library molecule with an affinity for a GS of <1E−9 M.

A3.6.2.7: A DNA-encoded peptide library, a medium comprising >80% organic solvent, a GS, and a library molecule with an affinity for a GS of <1E−10 M.

A3.6.2.8: A DNA-encoded peptide library, a medium comprising >80% organic solvent, a GS, and a library molecule with an affinity for a GS of <1E−12 M.

A3.6.3.1: A DNA-encoded peptide library, a medium comprising >80% organic solvent, a SWNT, and a library molecule with an affinity for a SWNT of <1E−2 M.

A3.6.3.2: A DNA-encoded peptide library, a medium comprising >80% organic solvent, a SWNT, and a library molecule with an affinity for a SWNT of <1E−4 M.

A3.6.3.3: A DNA-encoded peptide library, a medium comprising >80% organic solvent, a SWNT, and a library molecule with an affinity for a SWNT of <1E−6 M.

A3.6.3.4: A DNA-encoded peptide library, a medium comprising >80% organic solvent, a SWNT, and a library molecule with an affinity for a SWNT of <1E−7 M.

A3.6.3.5: A DNA-encoded peptide library, a medium comprising >80% organic solvent, a SWNT, and a library molecule with an affinity for a SWNT of <1E−8 M.

A3.6.3.6: A DNA-encoded peptide library, a medium comprising >80% organic solvent, a SWNT, and a library molecule with an affinity for a SWNT of <1E−9 M.

A3.6.3.7: A DNA-encoded peptide library, a medium comprising >80% organic solvent, a SWNT, and a library molecule with an affinity for a SWNT of <1E−10 M.

A3.6.3.8: A DNA-encoded peptide library, a medium comprising >80% organic solvent, a SWNT, and a library molecule with an affinity for a SWNT of <1E−12 M.

A3.6.4.1: A DNA-encoded peptide library, a medium comprising >80% organic solvent, a MWNT, and a library molecule with an affinity for a MWNT of <1E−2 M.

A3.6.4.2: A DNA-encoded peptide library, a medium comprising >80% organic solvent, a MWNT, and a library molecule with an affinity for a MWNT of <1E−4 M.

A3.6.4.3: A DNA-encoded peptide library, a medium comprising >80% organic solvent, a MWNT, and a library molecule with an affinity for a MWNT of <1E−6 M.

A3.6.4.4: A DNA-encoded peptide library, a medium comprising >80% organic solvent, a MWNT, and a library molecule with an affinity for a MWNT of <1E−7 M.

A3.6.4.5: A DNA-encoded peptide library, a medium comprising >80% organic solvent, a MWNT, and a library molecule with an affinity for a MWNT of <1E−8 M.

A3.6.4.6: A DNA-encoded peptide library, a medium comprising >80% organic solvent, a MWNT, and a library molecule with an affinity for a MWNT of <1E−9 M.

A3.6.4.7: A DNA-encoded peptide library, a medium comprising >80% organic solvent, a MWNT, and a library molecule with an affinity for a MWNT of <1E−10 M.

A3.6.4.8: A DNA-encoded peptide library, a medium comprising >80% organic solvent, a MWNT, and a library molecule with an affinity for a MWNT of <1E−12 M.

A3.6.5.1: A DNA-encoded peptide library, a medium comprising >80% organic solvent, a nanotube, and a library molecule with an affinity for a nanotube of <1E−2 M.

A3.6.5.2: A DNA-encoded peptide library, a medium comprising >80% organic solvent, a nanotube, and a library molecule with an affinity for a nanotube of <1E−4 M.

A3.6.5.3: A DNA-encoded peptide library, a medium comprising >80% organic solvent, a nanotube, and a library molecule with an affinity for a nanotube of <1E−6 M.

A3.6.5.4: A DNA-encoded peptide library, a medium comprising >80% organic solvent, a nanotube, and a library molecule with an affinity for a nanotube of <1E−7 M.

A3.6.5.5: A DNA-encoded peptide library, a medium comprising >80% organic solvent, a nanotube, and a library molecule with an affinity for a nanotube of <1E−8 M.

A3.6.5.6: A DNA-encoded peptide library, a medium comprising >80% organic solvent, a nanotube, and a library molecule with an affinity for a nanotube of <1E−9 M.

A3.6.5.7: A DNA-encoded peptide library, a medium comprising >80% organic solvent, a nanotube, and a library molecule with an affinity for a nanotube of <1E−10 M.

A3.6.5.8: A DNA-encoded peptide library, a medium comprising >80% organic solvent, a nanotube, and a library molecule with an affinity for a nanotube of <1E−12 M.

A3.6.6.1: A DNA-encoded peptide library, a medium comprising >80% organic solvent, a fullerene, and a library molecule with an affinity for a fullerene of <1E−2 M.

A3.6.6.2: A DNA-encoded peptide library, a medium comprising >80% organic solvent, a fullerene, and a library molecule with an affinity for a fullerene of <1E−4 M.

A3.6.6.3: A DNA-encoded peptide library, a medium comprising >80% organic solvent, a fullerene, and a library molecule with an affinity for a fullerene of <1E−6 M.

A3.6.6.4: A DNA-encoded peptide library, a medium comprising >80% organic solvent, a fullerene, and a library molecule with an affinity for a fullerene of <1E−7 M.

A3.6.6.5: A DNA-encoded peptide library, a medium comprising >80% organic solvent, a fullerene, and a library molecule with an affinity for a fullerene of <1E−8 M.

A3.6.6.6: A DNA-encoded peptide library, a medium comprising >80% organic solvent, a fullerene, and a library molecule with an affinity for a fullerene of <1E−9 M.

A3.6.6.7: A DNA-encoded peptide library, a medium comprising >80% organic solvent, a fullerene, and a library molecule with an affinity for a fullerene of <1E−10 M.

A3.6.6.8: A DNA-encoded peptide library, a medium comprising >80% organic solvent, a fullerene, and a library molecule with an affinity for a fullerene of <1E−12 M.

A3.6.7.1: A DNA-encoded peptide library, a medium comprising >80% organic solvent, a BNNT, and a library molecule with an affinity for a BNNT of <1E−2 M.

A3.6.7.2: A DNA-encoded peptide library, a medium comprising >80% organic solvent, a BNNT, and a library molecule with an affinity for a BNNT of <1E−4 M.

A3.6.7.3: A DNA-encoded peptide library, a medium comprising >80% organic solvent, a BNNT, and a library molecule with an affinity for a BNNT of <1E−6 M.

A3.6.7.4: A DNA-encoded peptide library, a medium comprising >80% organic solvent, a BNNT, and a library molecule with an affinity for a BNNT of <1E−7 M.

A3.6.7.5: A DNA-encoded peptide library, a medium comprising >80% organic solvent, a BNNT, and a library molecule with an affinity for a BNNT of <1E−8 M.

A3.6.7.6: A DNA-encoded peptide library, a medium comprising >80% organic solvent, a BNNT, and a library molecule with an affinity for a BNNT of <1E−9 M.

A3.6.7.7: A DNA-encoded peptide library, a medium comprising >80% organic solvent, a BNNT, and a library molecule with an affinity for a BNNT of <1E−10 M.

A3.6.7.8: A DNA-encoded peptide library, a medium comprising >80% organic solvent, a BNNT, and a library molecule with an affinity for a BNNT of <1E−12 M.

A3.7.1.1: A DNA-encoded peptide library, a medium comprising >80% organic solvent and a soluble polymer, a CNT, and a library molecule with an affinity for a CNT of <1E−2 M.

A3.7.1.2: A DNA-encoded peptide library, a medium comprising >80% organic solvent and a soluble polymer, a CNT, and a library molecule with an affinity for a CNT of <1E−4 M.

A3.7.1.3: A DNA-encoded peptide library, a medium comprising >80% organic solvent and a soluble polymer, a CNT, and a library molecule with an affinity for a CNT of <1E−6 M.

A3.7.1.4: A DNA-encoded peptide library, a medium comprising >80% organic solvent and a soluble polymer, a CNT, and a library molecule with an affinity for a CNT of <1E−7 M.

A3.7.1.5: A DNA-encoded peptide library, a medium comprising >80% organic solvent and a soluble polymer, a CNT, and a library molecule with an affinity for a CNT of <1E−8 M.

A3.7.1.6: A DNA-encoded peptide library, a medium comprising >80% organic solvent and a soluble polymer, a CNT, and a library molecule with an affinity for a CNT of <1E−9 M.

A3.7.1.7: A DNA-encoded peptide library, a medium comprising >80% organic solvent and a soluble polymer, a CNT, and a library molecule with an affinity for a CNT of <1E−10 M.

A3.7.1.8: A DNA-encoded peptide library, a medium comprising >80% organic solvent and a soluble polymer, a CNT, and a library molecule with an affinity for a CNT of <1E−12 M.

A3.7.2.1: A DNA-encoded peptide library, a medium comprising >80% organic solvent and a soluble polymer, a GS, and a library molecule with an affinity for a GS of <1E−2 M.

A3.7.2.2: A DNA-encoded peptide library, a medium comprising >80% organic solvent and a soluble polymer, a GS, and a library molecule with an affinity for a GS of <1E−4 M.

A3.7.2.3: A DNA-encoded peptide library, a medium comprising >80% organic solvent and a soluble polymer, a GS, and a library molecule with an affinity for a GS of <1E−6 M.

A3.7.2.4: A DNA-encoded peptide library, a medium comprising >80% organic solvent and a soluble polymer, a GS, and a library molecule with an affinity for a GS of <1E−7 M.

A3.7.2.5: A DNA-encoded peptide library, a medium comprising >80% organic solvent and a soluble polymer, a GS, and a library molecule with an affinity for a GS of <1E−8 M.

A3.7.2.6: A DNA-encoded peptide library, a medium comprising >80% organic solvent and a soluble polymer, a GS, and a library molecule with an affinity for a GS of <1E−9 M.

A3.7.2.7: A DNA-encoded peptide library, a medium comprising >80% organic solvent and a soluble polymer, a GS, and a library molecule with an affinity for a GS of <1E−10 M.

A3.7.2.8: A DNA-encoded peptide library, a medium comprising >80% organic solvent and a soluble polymer, a GS, and a library molecule with an affinity for a GS of <1E−12 M.

A3.7.3.1: A DNA-encoded peptide library, a medium comprising >80% organic solvent and a soluble polymer, a SWNT, and a library molecule with an affinity for a SWNT of <1E−2 M.

A3.7.3.2: A DNA-encoded peptide library, a medium comprising >80% organic solvent and a soluble polymer, a SWNT, and a library molecule with an affinity for a SWNT of <1E−4 M.

A3.7.3.3: A DNA-encoded peptide library, a medium comprising >80% organic solvent and a soluble polymer, a SWNT, and a library molecule with an affinity for a SWNT of <1E−6 M.

A3.7.3.4: A DNA-encoded peptide library, a medium comprising >80% organic solvent and a soluble polymer, a SWNT, and a library molecule with an affinity for a SWNT of <1E−7 M.

A3.7.3.5: A DNA-encoded peptide library, a medium comprising >80% organic solvent and a soluble polymer, a SWNT, and a library molecule with an affinity for a SWNT of <1E−8 M.

A3.7.3.6: A DNA-encoded peptide library, a medium comprising >80% organic solvent and a soluble polymer, a SWNT, and a library molecule with an affinity for a SWNT of <1E−9 M.

A3.7.3.7: A DNA-encoded peptide library, a medium comprising >80% organic solvent and a soluble polymer, a SWNT, and a library molecule with an affinity for a SWNT of <1E−10 M.

A3.7.3.8: A DNA-encoded peptide library, a medium comprising >80% organic solvent and a soluble polymer, a SWNT, and a library molecule with an affinity for a SWNT of <1E−12 M.

A3.7.4.1: A DNA-encoded peptide library, a medium comprising >80% organic solvent and a soluble polymer, a MWNT, and a library molecule with an affinity for a MWNT of <1E−2 M.

A3.7.4.2: A DNA-encoded peptide library, a medium comprising >80% organic solvent and a soluble polymer, a MWNT, and a library molecule with an affinity for a MWNT of <1E−4 M.

A3.7.4.3: A DNA-encoded peptide library, a medium comprising >80% organic solvent and a soluble polymer, a MWNT, and a library molecule with an affinity for a MWNT of <1E−6 M.

A3.7.4.4: A DNA-encoded peptide library, a medium comprising >80% organic solvent and a soluble polymer, a MWNT, and a library molecule with an affinity for a MWNT of <1E−7 M.

A3.7.4.5: A DNA-encoded peptide library, a medium comprising >80% organic solvent and a soluble polymer, a MWNT, and a library molecule with an affinity for a MWNT of <1E−8 M.

A3.7.4.6: A DNA-encoded peptide library, a medium comprising >80% organic solvent and a soluble polymer, a MWNT, and a library molecule with an affinity for a MWNT of <1E−9 M.

A3.7.4.7: A DNA-encoded peptide library, a medium comprising >80% organic solvent and a soluble polymer, a MWNT, and a library molecule with an affinity for a MWNT of <1E−10 M.

A3.7.4.8: A DNA-encoded peptide library, a medium comprising >80% organic solvent and a soluble polymer, a MWNT, and a library molecule with an affinity for a MWNT of <1E−12 M.

A3.7.5.1: A DNA-encoded peptide library, a medium comprising >80% organic solvent and a soluble polymer, a nanotube, and a library molecule with an affinity for a nanotube of <1E−2 M.

A3.7.5.2: A DNA-encoded peptide library, a medium comprising >80% organic solvent and a soluble polymer, a nanotube, and a library molecule with an affinity for a nanotube of <1E−4 M.

A3.7.5.3: A DNA-encoded peptide library, a medium comprising >80% organic solvent and a soluble polymer, a nanotube, and a library molecule with an affinity for a nanotube of <1E−6 M.

A3.7.5.4: A DNA-encoded peptide library, a medium comprising >80% organic solvent and a soluble polymer, a nanotube, and a library molecule with an affinity for a nanotube of <1E−7 M.

A3.7.5.5: A DNA-encoded peptide library, a medium comprising >80% organic solvent and a soluble polymer, a nanotube, and a library molecule with an affinity for a nanotube of <1E−8 M.

A3.7.5.6: A DNA-encoded peptide library, a medium comprising >80% organic solvent and a soluble polymer, a nanotube, and a library molecule with an affinity for a nanotube of <1E−9 M.

A3.7.5.7: A DNA-encoded peptide library, a medium comprising >80% organic solvent and a soluble polymer, a nanotube, and a library molecule with an affinity for a nanotube of <1E−10 M.

A3.7.5.8: A DNA-encoded peptide library, a medium comprising >80% organic solvent and a soluble polymer, a nanotube, and a library molecule with an affinity for a nanotube of <1E−12 M.

A3.7.6.1: A DNA-encoded peptide library, a medium comprising >80% organic solvent and a soluble polymer, a fullerene, and a library molecule with an affinity for a fullerene of <1E−2 M.

A3.7.6.2: A DNA-encoded peptide library, a medium comprising >80% organic solvent and a soluble polymer, a fullerene, and a library molecule with an affinity for a fullerene of <1E−4 M.

A3.7.6.3: A DNA-encoded peptide library, a medium comprising >80% organic solvent and a soluble polymer, a fullerene, and a library molecule with an affinity for a fullerene of <1E−6 M.

A3.7.6.4: A DNA-encoded peptide library, a medium comprising >80% organic solvent and a soluble polymer, a fullerene, and a library molecule with an affinity for a fullerene of <1E−7 M.

A3.7.6.5: A DNA-encoded peptide library, a medium comprising >80% organic solvent and a soluble polymer, a fullerene, and a library molecule with an affinity for a fullerene of <1E−8 M.

A3.7.6.6: A DNA-encoded peptide library, a medium comprising >80% organic solvent and a soluble polymer, a fullerene, and a library molecule with an affinity for a fullerene of <1E−9 M.

A3.7.6.7: A DNA-encoded peptide library, a medium comprising >80% organic solvent and a soluble polymer, a fullerene, and a library molecule with an affinity for a fullerene of <1E−10 M.

A3.7.6.8: A DNA-encoded peptide library, a medium comprising >80% organic solvent and a soluble polymer, a fullerene, and a library molecule with an affinity for a fullerene of <1E−12 M.

A3.7.7.1: A DNA-encoded peptide library, a medium comprising >80% organic solvent and a soluble polymer, a BNNT, and a library molecule with an affinity for a BNNT of <1E−2 M.

A3.7.7.2: A DNA-encoded peptide library, a medium comprising >80% organic solvent and a soluble polymer, a BNNT, and a library molecule with an affinity for a BNNT of <1E−4 M.

A3.7.7.3: A DNA-encoded peptide library, a medium comprising >80% organic solvent and a soluble polymer, a BNNT, and a library molecule with an affinity for a BNNT of <1E−6 M.

A3.7.7.4: A DNA-encoded peptide library, a medium comprising >80% organic solvent and a soluble polymer, a BNNT, and a library molecule with an affinity for a BNNT of <1E−7 M.

A3.7.7.5: A DNA-encoded peptide library, a medium comprising >80% organic solvent and a soluble polymer, a BNNT, and a library molecule with an affinity for a BNNT of <1E−8 M.

A3.7.7.6: A DNA-encoded peptide library, a medium comprising >80% organic solvent and a soluble polymer, a BNNT, and a library molecule with an affinity for a BNNT of <1E−9 M.

A3.7.7.7: A DNA-encoded peptide library, a medium comprising >80% organic solvent and a soluble polymer, a BNNT, and a library molecule with an affinity for a BNNT of <1E−10 M.

A3.7.7.8: A DNA-encoded peptide library, a medium comprising >80% organic solvent and a soluble polymer, a BNNT, and a library molecule with an affinity for a BNNT of <1E−12 M.

A3.8.1.1: A DNA-encoded peptide library, a medium comprising >95% organic solvent, a CNT, and a library molecule with an affinity for a CNT of <1E−2 M.

A3.8.1.2: A DNA-encoded peptide library, a medium comprising >95% organic solvent, a CNT, and a library molecule with an affinity for a CNT of <1E−4 M.

A3.8.1.3: A DNA-encoded peptide library, a medium comprising >95% organic solvent, a CNT, and a library molecule with an affinity for a CNT of <1E−6 M.

A3.8.1.4: A DNA-encoded peptide library, a medium comprising >95% organic solvent, a CNT, and a library molecule with an affinity for a CNT of <1E−7 M.

A3.8.1.5: A DNA-encoded peptide library, a medium comprising >95% organic solvent, a CNT, and a library molecule with an affinity for a CNT of <1E−8 M.

A3.8.1.6: A DNA-encoded peptide library, a medium comprising >95% organic solvent, a CNT, and a library molecule with an affinity for a CNT of <1E−9 M.

A3.8.1.7: A DNA-encoded peptide library, a medium comprising >95% organic solvent, a CNT, and a library molecule with an affinity for a CNT of <1E−10 M.

A3.8.1.8: A DNA-encoded peptide library, a medium comprising >95% organic solvent, a CNT, and a library molecule with an affinity for a CNT of <1E−12 M.

A3.8.2.1: A DNA-encoded peptide library, a medium comprising >95% organic solvent, a GS, and a library molecule with an affinity for a GS of <1E−2 M.

A3.8.2.2: A DNA-encoded peptide library, a medium comprising >95% organic solvent, a GS, and a library molecule with an affinity for a GS of <1E−4 M.

A3.8.2.3: A DNA-encoded peptide library, a medium comprising >95% organic solvent, a GS, and a library molecule with an affinity for a GS of <1E−6 M.

A3.8.2.4: A DNA-encoded peptide library, a medium comprising >95% organic solvent, a GS, and a library molecule with an affinity for a GS of <1E−7 M.

A3.8.2.5: A DNA-encoded peptide library, a medium comprising >95% organic solvent, a GS, and a library molecule with an affinity for a GS of <1E−8 M.

A3.8.2.6: A DNA-encoded peptide library, a medium comprising >95% organic solvent, a GS, and a library molecule with an affinity for a GS of <1E−9 M.

A3.8.2.7: A DNA-encoded peptide library, a medium comprising >95% organic solvent, a GS, and a library molecule with an affinity for a GS of <1E−10 M.

A3.8.2.8: A DNA-encoded peptide library, a medium comprising >95% organic solvent, a GS, and a library molecule with an affinity for a GS of <1E−12 M.

A3.8.3.1: A DNA-encoded peptide library, a medium comprising >95% organic solvent, a SWNT, and a library molecule with an affinity for a SWNT of <1E−2 M.

A3.8.3.2: A DNA-encoded peptide library, a medium comprising >95% organic solvent, a SWNT, and a library molecule with an affinity for a SWNT of <1E−4 M.

A3.8.3.3: A DNA-encoded peptide library, a medium comprising >95% organic solvent, a SWNT, and a library molecule with an affinity for a SWNT of <1E−6 M.

A3.8.3.4: A DNA-encoded peptide library, a medium comprising >95% organic solvent, a SWNT, and a library molecule with an affinity for a SWNT of <1E−7 M.

A3.8.3.5: A DNA-encoded peptide library, a medium comprising >95% organic solvent, a SWNT, and a library molecule with an affinity for a SWNT of <1E−8 M.

A3.8.3.6: A DNA-encoded peptide library, a medium comprising >95% organic solvent, a SWNT, and a library molecule with an affinity for a SWNT of <1E−9 M.

A3.8.3.7: A DNA-encoded peptide library, a medium comprising >95% organic solvent, a SWNT, and a library molecule with an affinity for a SWNT of <1E−10 M.

A3.8.3.8: A DNA-encoded peptide library, a medium comprising >95% organic solvent, a SWNT, and a library molecule with an affinity for a SWNT of <1E−12 M.

A3.8.4.1: A DNA-encoded peptide library, a medium comprising >95% organic solvent, a MWNT, and a library molecule with an affinity for a MWNT of <1E−2 M.

A3.8.4.2: A DNA-encoded peptide library, a medium comprising >95% organic solvent, a MWNT, and a library molecule with an affinity for a MWNT of <1E−4 M.

A3.8.4.3: A DNA-encoded peptide library, a medium comprising >95% organic solvent, a MWNT, and a library molecule with an affinity for a MWNT of <1E−6 M.

A3.8.4.4: A DNA-encoded peptide library, a medium comprising >95% organic solvent, a MWNT, and a library molecule with an affinity for a MWNT of <1E−7 M.

A3.8.4.5: A DNA-encoded peptide library, a medium comprising >95% organic solvent, a MWNT, and a library molecule with an affinity for a MWNT of <1E−8 M.

A3.8.4.6: A DNA-encoded peptide library, a medium comprising >95% organic solvent, a MWNT, and a library molecule with an affinity for a MWNT of <1E−9 M.

A3.8.4.7: A DNA-encoded peptide library, a medium comprising >95% organic solvent, a MWNT, and a library molecule with an affinity for a MWNT of <1E−10 M.

A3.8.4.8: A DNA-encoded peptide library, a medium comprising >95% organic solvent, a MWNT, and a library molecule with an affinity for a MWNT of <1E−12 M.

A3.8.5.1: A DNA-encoded peptide library, a medium comprising >95% organic solvent, a nanotube, and a library molecule with an affinity for a nanotube of <1E−2 M.

A3.8.5.2: A DNA-encoded peptide library, a medium comprising >95% organic solvent, a nanotube, and a library molecule with an affinity for a nanotube of <1E−4 M.

A3.8.5.3: A DNA-encoded peptide library, a medium comprising >95% organic solvent, a nanotube, and a library molecule with an affinity for a nanotube of <1E−6 M.

A3.8.5.4: A DNA-encoded peptide library, a medium comprising >95% organic solvent, a nanotube, and a library molecule with an affinity for a nanotube of <1E−7 M.

A3.8.5.5: A DNA-encoded peptide library, a medium comprising >95% organic solvent, a nanotube, and a library molecule with an affinity for a nanotube of <1E−8 M.

A3.8.5.6: A DNA-encoded peptide library, a medium comprising >95% organic solvent, a nanotube, and a library molecule with an affinity for a nanotube of <1E−9 M.

A3.8.5.7: A DNA-encoded peptide library, a medium comprising >95% organic solvent, a nanotube, and a library molecule with an affinity for a nanotube of <1E−10 M.

A3.8.5.8: A DNA-encoded peptide library, a medium comprising >95% organic solvent, a nanotube, and a library molecule with an affinity for a nanotube of <1E−12 M.

A3.8.6.1: A DNA-encoded peptide library, a medium comprising >95% organic solvent, a fullerene, and a library molecule with an affinity for a fullerene of <1E−2 M.

A3.8.6.2: A DNA-encoded peptide library, a medium comprising >95% organic solvent, a fullerene, and a library molecule with an affinity for a fullerene of <1E−4 M.

A3.8.6.3: A DNA-encoded peptide library, a medium comprising >95% organic solvent, a fullerene, and a library molecule with an affinity for a fullerene of <1E−6 M.

A3.8.6.4: A DNA-encoded peptide library, a medium comprising >95% organic solvent, a fullerene, and a library molecule with an affinity for a fullerene of <1E−7 M.

A3.8.6.5: A DNA-encoded peptide library, a medium comprising >95% organic solvent, a fullerene, and a library molecule with an affinity for a fullerene of <1E−8 M.

A3.8.6.6: A DNA-encoded peptide library, a medium comprising >95% organic solvent, a fullerene, and a library molecule with an affinity for a fullerene of <1E−9 M.

A3.8.6.7: A DNA-encoded peptide library, a medium comprising >95% organic solvent, a fullerene, and a library molecule with an affinity for a fullerene of <1E−10 M.

A3.8.6.8: A DNA-encoded peptide library, a medium comprising >95% organic solvent, a fullerene, and a library molecule with an affinity for a fullerene of <1E−12 M.

A3.8.7.1: A DNA-encoded peptide library, a medium comprising >95% organic solvent, a BNNT, and a library molecule with an affinity for a BNNT of <1E−2 M.

A3.8.7.2: A DNA-encoded peptide library, a medium comprising >95% organic solvent, a BNNT, and a library molecule with an affinity for a BNNT of <1E−4 M.

A3.8.7.3: A DNA-encoded peptide library, a medium comprising >95% organic solvent, a BNNT, and a library molecule with an affinity for a BNNT of <1E−6 M.

A3.8.7.4: A DNA-encoded peptide library, a medium comprising >95% organic solvent, a BNNT, and a library molecule with an affinity for a BNNT of <1E−7 M.

A3.8.7.5: A DNA-encoded peptide library, a medium comprising >95% organic solvent, a BNNT, and a library molecule with an affinity for a BNNT of <1E−8 M.

A3.8.7.6: A DNA-encoded peptide library, a medium comprising >95% organic solvent, a BNNT, and a library molecule with an affinity for a BNNT of <1E−9 M.

A3.8.7.7: A DNA-encoded peptide library, a medium comprising >95% organic solvent, a BNNT, and a library molecule with an affinity for a BNNT of <1E−10 M.

A3.8.7.8: A DNA-encoded peptide library, a medium comprising >95% organic solvent, a BNNT, and a library molecule with an affinity for a BNNT of <1E−12 M.

A3.9.1.1: A DNA-encoded peptide library, a medium comprising >95% organic solvent and a soluble polymer, a CNT, and a library molecule with an affinity for a CNT of <1E−2 M.

A3.9.1.2: A DNA-encoded peptide library, a medium comprising >95% organic solvent and a soluble polymer, a CNT, and a library molecule with an affinity for a CNT of <1E−4 M.

A3.9.1.3: A DNA-encoded peptide library, a medium comprising >95% organic solvent and a soluble polymer, a CNT, and a library molecule with an affinity for a CNT of <1E−6 M.

A3.9.1.4: A DNA-encoded peptide library, a medium comprising >95% organic solvent and a soluble polymer, a CNT, and a library molecule with an affinity for a CNT of <1E−7 M.

A3.9.1.5: A DNA-encoded peptide library, a medium comprising >95% organic solvent and a soluble polymer, a CNT, and a library molecule with an affinity for a CNT of <1E−8 M.

A3.9.1.6: A DNA-encoded peptide library, a medium comprising >95% organic solvent and a soluble polymer, a CNT, and a library molecule with an affinity for a CNT of <1E−9 M.

A3.9.1.7: A DNA-encoded peptide library, a medium comprising >95% organic solvent and a soluble polymer, a CNT, and a library molecule with an affinity for a CNT of <1E−10 M.

A3.9.1.8: A DNA-encoded peptide library, a medium comprising >95% organic solvent and a soluble polymer, a CNT, and a library molecule with an affinity for a CNT of <1E−12 M.

A3.9.2.1: A DNA-encoded peptide library, a medium comprising >95% organic solvent and a soluble polymer, a GS, and a library molecule with an affinity for a GS of <1E−2 M.

A3.9.2.2: A DNA-encoded peptide library, a medium comprising >95% organic solvent and a soluble polymer, a GS, and a library molecule with an affinity for a GS of <1E−4 M.

A3.9.2.3: A DNA-encoded peptide library, a medium comprising >95% organic solvent and a soluble polymer, a GS, and a library molecule with an affinity for a GS of <1E−6 M.

A3.9.2.4: A DNA-encoded peptide library, a medium comprising >95% organic solvent and a soluble polymer, a GS, and a library molecule with an affinity for a GS of <1E−7 M.

A3.9.2.5: A DNA-encoded peptide library, a medium comprising >95% organic solvent and a soluble polymer, a GS, and a library molecule with an affinity for a GS of <1E−8 M.

A3.9.2.6: A DNA-encoded peptide library, a medium comprising >95% organic solvent and a soluble polymer, a GS, and a library molecule with an affinity for a GS of <1E−9 M.

A3.9.2.7: A DNA-encoded peptide library, a medium comprising >95% organic solvent and a soluble polymer, a GS, and a library molecule with an affinity for a GS of <1E−10 M.

A3.9.2.8: A DNA-encoded peptide library, a medium comprising >95% organic solvent and a soluble polymer, a GS, and a library molecule with an affinity for a GS of <1E−12 M.

A3.9.3.1: A DNA-encoded peptide library, a medium comprising >95% organic solvent and a soluble polymer, a SWNT, and a library molecule with an affinity for a SWNT of <1E−2 M.

A3.9.3.2: A DNA-encoded peptide library, a medium comprising >95% organic solvent and a soluble polymer, a SWNT, and a library molecule with an affinity for a SWNT of <1E−4 M.

A3.9.3.3: A DNA-encoded peptide library, a medium comprising >95% organic solvent and a soluble polymer, a SWNT, and a library molecule with an affinity for a SWNT of <1E−6 M.

A3.9.3.4: A DNA-encoded peptide library, a medium comprising >95% organic solvent and a soluble polymer, a SWNT, and a library molecule with an affinity for a SWNT of <1E−7 M.

A3.9.3.5: A DNA-encoded peptide library, a medium comprising >95% organic solvent and a soluble polymer, a SWNT, and a library molecule with an affinity for a SWNT of <1E−8 M.

A3.9.3.6: A DNA-encoded peptide library, a medium comprising >95% organic solvent and a soluble polymer, a SWNT, and a library molecule with an affinity for a SWNT of <1E−9 M.

A3.9.3.7: A DNA-encoded peptide library, a medium comprising >95% organic solvent and a soluble polymer, a SWNT, and a library molecule with an affinity for a SWNT of <1E−10 M.

A3.9.3.8: A DNA-encoded peptide library, a medium comprising >95% organic solvent and a soluble polymer, a SWNT, and a library molecule with an affinity for a SWNT of <1E−12 M.

A3.9.4.1: A DNA-encoded peptide library, a medium comprising >95% organic solvent and a soluble polymer, a MWNT, and a library molecule with an affinity for a MWNT of <1E−2 M.

A3.9.4.2: A DNA-encoded peptide library, a medium comprising >95% organic solvent and a soluble polymer, a MWNT, and a library molecule with an affinity for a MWNT of <1E−4 M.

A3.9.4.3: A DNA-encoded peptide library, a medium comprising >95% organic solvent and a soluble polymer, a MWNT, and a library molecule with an affinity for a MWNT of <1E−6 M.

A3.9.4.4: A DNA-encoded peptide library, a medium comprising >95% organic solvent and a soluble polymer, a MWNT, and a library molecule with an affinity for a MWNT of <1E−7 M.

A3.9.4.5: A DNA-encoded peptide library, a medium comprising >95% organic solvent and a soluble polymer, a MWNT, and a library molecule with an affinity for a MWNT of <1E−8 M.

A3.9.4.6: A DNA-encoded peptide library, a medium comprising >95% organic solvent and a soluble polymer, a MWNT, and a library molecule with an affinity for a MWNT of <1E−9 M.

A3.9.4.7: A DNA-encoded peptide library, a medium comprising >95% organic solvent and a soluble polymer, a MWNT, and a library molecule with an affinity for a MWNT of <1E−10 M.

A3.9.4.8: A DNA-encoded peptide library, a medium comprising >95% organic solvent and a soluble polymer, a MWNT, and a library molecule with an affinity for a MWNT of <1E−12 M.

A3.9.5.1: A DNA-encoded peptide library, a medium comprising >95% organic solvent and a soluble polymer, a nanotube, and a library molecule with an affinity for a nanotube of <1E−2 M.

A3.9.5.2: A DNA-encoded peptide library, a medium comprising >95% organic solvent and a soluble polymer, a nanotube, and a library molecule with an affinity for a nanotube of <1E−4 M.

A3.9.5.3: A DNA-encoded peptide library, a medium comprising >95% organic solvent and a soluble polymer, a nanotube, and a library molecule with an affinity for a nanotube of <1E−6 M.

A3.9.5.4: A DNA-encoded peptide library, a medium comprising >95% organic solvent and a soluble polymer, a nanotube, and a library molecule with an affinity for a nanotube of <1E−7 M.

A3.9.5.5: A DNA-encoded peptide library, a medium comprising >95% organic solvent and a soluble polymer, a nanotube, and a library molecule with an affinity for a nanotube of <1E−8 M.

A3.9.5.6: A DNA-encoded peptide library, a medium comprising >95% organic solvent and a soluble polymer, a nanotube, and a library molecule with an affinity for a nanotube of <1E−9 M.

A3.9.5.7: A DNA-encoded peptide library, a medium comprising >95% organic solvent and a soluble polymer, a nanotube, and a library molecule with an affinity for a nanotube of <1E−10 M.

A3.9.5.8: A DNA-encoded peptide library, a medium comprising >95% organic solvent and a soluble polymer, a nanotube, and a library molecule with an affinity for a nanotube of <1E−12 M.

A3.9.6.1: A DNA-encoded peptide library, a medium comprising >95% organic solvent and a soluble polymer, a fullerene, and a library molecule with an affinity for a fullerene of <1E−2 M.

A3.9.6.2: A DNA-encoded peptide library, a medium comprising >95% organic solvent and a soluble polymer, a fullerene, and a library molecule with an affinity for a fullerene of <1E−4 M.

A3.9.6.3: A DNA-encoded peptide library, a medium comprising >95% organic solvent and a soluble polymer, a fullerene, and a library molecule with an affinity for a fullerene of <1E−6 M.

A3.9.6.4: A DNA-encoded peptide library, a medium comprising >95% organic solvent and a soluble polymer, a fullerene, and a library molecule with an affinity for a fullerene of <1E−7 M.

A3.9.6.5: A DNA-encoded peptide library, a medium comprising >95% organic solvent and a soluble polymer, a fullerene, and a library molecule with an affinity for a fullerene of <1E−8 M.

A3.9.6.6: A DNA-encoded peptide library, a medium comprising >95% organic solvent and a soluble polymer, a fullerene, and a library molecule with an affinity for a fullerene of <1E−9 M.

A3.9.6.7: A DNA-encoded peptide library, a medium comprising >95% organic solvent and a soluble polymer, a fullerene, and a library molecule with an affinity for a fullerene of <1E−10 M.

A3.9.6.8: A DNA-encoded peptide library, a medium comprising >95% organic solvent and a soluble polymer, a fullerene, and a library molecule with an affinity for a fullerene of <1E−12 M.

A3.9.7.1: A DNA-encoded peptide library, a medium comprising >95% organic solvent and a soluble polymer, a BNNT, and a library molecule with an affinity for a BNNT of <1E−2 M.

A3.9.7.2: A DNA-encoded peptide library, a medium comprising >95% organic solvent and a soluble polymer, a BNNT, and a library molecule with an affinity for a BNNT of <1E−4 M.

A3.9.7.3: A DNA-encoded peptide library, a medium comprising >95% organic solvent and a soluble polymer, a BNNT, and a library molecule with an affinity for a BNNT of <1E−6 M.

A3.9.7.4: A DNA-encoded peptide library, a medium comprising >95% organic solvent and a soluble polymer, a BNNT, and a library molecule with an affinity for a BNNT of <1E−7 M.

A3.9.7.5: A DNA-encoded peptide library, a medium comprising >95% organic solvent and a soluble polymer, a BNNT, and a library molecule with an affinity for a BNNT of <1E−8 M.

A3.9.7.6: A DNA-encoded peptide library, a medium comprising >95% organic solvent and a soluble polymer, a BNNT, and a library molecule with an affinity for a BNNT of <1E−9 M.

A3.9.7.7: A DNA-encoded peptide library, a medium comprising >95% organic solvent and a soluble polymer, a BNNT, and a library molecule with an affinity for a BNNT of <1E−10 M.

A3.9.7.8: A DNA-encoded peptide library, a medium comprising >95% organic solvent and a soluble polymer, a BNNT, and a library molecule with an affinity for a BNNT of <1E−12 M.

A4.1.1.1: A phage-encoded peptide library, a medium comprising an organic solvent, a CNT, and a library molecule with an affinity for a CNT of <1E−2 M.

A4.1.1.2: A phage-encoded peptide library, a medium comprising an organic solvent, a CNT, and a library molecule with an affinity for a CNT of <1E−4 M.

A4.1.1.3: A phage-encoded peptide library, a medium comprising an organic solvent, a CNT, and a library molecule with an affinity for a CNT of <1E−6 M.

A4.1.1.4: A phage-encoded peptide library, a medium comprising an organic solvent, a CNT, and a library molecule with an affinity for a CNT of <1E−7 M.

A4.1.1.5: A phage-encoded peptide library, a medium comprising an organic solvent, a CNT, and a library molecule with an affinity for a CNT of <1E−8 M.

A4.1.1.6: A phage-encoded peptide library, a medium comprising an organic solvent, a CNT, and a library molecule with an affinity for a CNT of <1E−9 M.

A4.1.1.7: A phage-encoded peptide library, a medium comprising an organic solvent, a CNT, and a library molecule with an affinity for a CNT of <1E−10 M.

A4.1.1.8: A phage-encoded peptide library, a medium comprising an organic solvent, a CNT, and a library molecule with an affinity for a CNT of <1E−12 M.

A4.1.2.1: A phage-encoded peptide library, a medium comprising an organic solvent, a GS, and a library molecule with an affinity for a GS of <1E−2 M.

A4.1.2.2: A phage-encoded peptide library, a medium comprising an organic solvent, a GS, and a library molecule with an affinity for a GS of <1E−4 M.

A4.1.2.3: A phage-encoded peptide library, a medium comprising an organic solvent, a GS, and a library molecule with an affinity for a GS of <1E−6 M.

A4.1.2.4: A phage-encoded peptide library, a medium comprising an organic solvent, a GS, and a library molecule with an affinity for a GS of <1E−7 M.

A4.1.2.5: A phage-encoded peptide library, a medium comprising an organic solvent, a GS, and a library molecule with an affinity for a GS of <1E−8 M.

A4.1.2.6: A phage-encoded peptide library, a medium comprising an organic solvent, a GS, and a library molecule with an affinity for a GS of <1E−9 M.

A4.1.2.7: A phage-encoded peptide library, a medium comprising an organic solvent, a GS, and a library molecule with an affinity for a GS of <1E−10 M.

A4.1.2.8: A phage-encoded peptide library, a medium comprising an organic solvent, a GS, and a library molecule with an affinity for a GS of <1E−12 M.

A4.1.3.1: A phage-encoded peptide library, a medium comprising an organic solvent, a SWNT, and a library molecule with an affinity for a SWNT of <1E−2 M.

A4.1.3.2: A phage-encoded peptide library, a medium comprising an organic solvent, a SWNT, and a library molecule with an affinity for a SWNT of <1E−4 M.

A4.1.3.3: A phage-encoded peptide library, a medium comprising an organic solvent, a SWNT, and a library molecule with an affinity for a SWNT of <1E−6 M.

A4.1.3.4: A phage-encoded peptide library, a medium comprising an organic solvent, a SWNT, and a library molecule with an affinity for a SWNT of <1E−7 M.

A4.1.3.5: A phage-encoded peptide library, a medium comprising an organic solvent, a SWNT, and a library molecule with an affinity for a SWNT of <1E−8 M.

A4.1.3.6: A phage-encoded peptide library, a medium comprising an organic solvent, a SWNT, and a library molecule with an affinity for a SWNT of <1E−9 M.

A4.1.3.7: A phage-encoded peptide library, a medium comprising an organic solvent, a SWNT, and a library molecule with an affinity for a SWNT of <1E−10 M.

A4.1.3.8: A phage-encoded peptide library, a medium comprising an organic solvent, a SWNT, and a library molecule with an affinity for a SWNT of <1E−12 M.

A4.1.4.1: A phage-encoded peptide library, a medium comprising an organic solvent, a MWNT, and a library molecule with an affinity for a MWNT of <1E−2 M.

A4.1.4.2: A phage-encoded peptide library, a medium comprising an organic solvent, a MWNT, and a library molecule with an affinity for a MWNT of <1E−4 M.

A4.1.4.3: A phage-encoded peptide library, a medium comprising an organic solvent, a MWNT, and a library molecule with an affinity for a MWNT of <1E−6 M.

A4.1.4.4: A phage-encoded peptide library, a medium comprising an organic solvent, a MWNT, and a library molecule with an affinity for a MWNT of <1E−7 M.

A4.1.4.5: A phage-encoded peptide library, a medium comprising an organic solvent, a MWNT, and a library molecule with an affinity for a MWNT of <1E−8 M.

A4.1.4.6: A phage-encoded peptide library, a medium comprising an organic solvent, a MWNT, and a library molecule with an affinity for a MWNT of <1E−9 M.

A4.1.4.7: A phage-encoded peptide library, a medium comprising an organic solvent, a MWNT, and a library molecule with an affinity for a MWNT of <1E−10 M.

A4.1.4.8: A phage-encoded peptide library, a medium comprising an organic solvent, a MWNT, and a library molecule with an affinity for a MWNT of <1E−12 M.

A4.1.5.1: A phage-encoded peptide library, a medium comprising an organic solvent, a nanotube, and a library molecule with an affinity for a nanotube of <1E−2 M.

A4.1.5.2: A phage-encoded peptide library, a medium comprising an organic solvent, a nanotube, and a library molecule with an affinity for a nanotube of <1E−4 M.

A4.1.5.3: A phage-encoded peptide library, a medium comprising an organic solvent, a nanotube, and a library molecule with an affinity for a nanotube of <1E−6 M.

A4.1.5.4: A phage-encoded peptide library, a medium comprising an organic solvent, a nanotube, and a library molecule with an affinity for a nanotube of <1E−7 M.

A4.1.5.5: A phage-encoded peptide library, a medium comprising an organic solvent, a nanotube, and a library molecule with an affinity for a nanotube of <1E−8 M.

A4.1.5.6: A phage-encoded peptide library, a medium comprising an organic solvent, a nanotube, and a library molecule with an affinity for a nanotube of <1E−9 M.

A4.1.5.7: A phage-encoded peptide library, a medium comprising an organic solvent, a nanotube, and a library molecule with an affinity for a nanotube of <1E−10 M.

A4.1.5.8: A phage-encoded peptide library, a medium comprising an organic solvent, a nanotube, and a library molecule with an affinity for a nanotube of <1E−12 M.

A4.1.6.1: A phage-encoded peptide library, a medium comprising an organic solvent, a fullerene, and a library molecule with an affinity for a fullerene of <1E−2 M.

A4.1.6.2: A phage-encoded peptide library, a medium comprising an organic solvent, a fullerene, and a library molecule with an affinity for a fullerene of <1E−4 M.

A4.1.6.3: A phage-encoded peptide library, a medium comprising an organic solvent, a fullerene, and a library molecule with an affinity for a fullerene of <1E−6 M.

A4.1.6.4: A phage-encoded peptide library, a medium comprising an organic solvent, a fullerene, and a library molecule with an affinity for a fullerene of <1E−7 M.

A4.1.6.5: A phage-encoded peptide library, a medium comprising an organic solvent, a fullerene, and a library molecule with an affinity for a fullerene of <1E−8 M.

A4.1.6.6: A phage-encoded peptide library, a medium comprising an organic solvent, a fullerene, and a library molecule with an affinity for a fullerene of <1E−9 M.

A4.1.6.7: A phage-encoded peptide library, a medium comprising an organic solvent, a fullerene, and a library molecule with an affinity for a fullerene of <1E−10 M.

A4.1.6.8: A phage-encoded peptide library, a medium comprising an organic solvent, a fullerene, and a library molecule with an affinity for a fullerene of <1E−12 M.

A4.1.7.1: A phage-encoded peptide library, a medium comprising an organic solvent, a BNNT, and a library molecule with an affinity for a BNNT of <1E−2 M.

A4.1.7.2: A phage-encoded peptide library, a medium comprising an organic solvent, a BNNT, and a library molecule with an affinity for a BNNT of <1E−4 M.

A4.1.7.3: A phage-encoded peptide library, a medium comprising an organic solvent, a BNNT, and a library molecule with an affinity for a BNNT of <1E−6 M.

A4.1.7.4: A phage-encoded peptide library, a medium comprising an organic solvent, a BNNT, and a library molecule with an affinity for a BNNT of <1E−7 M.

A4.1.7.5: A phage-encoded peptide library, a medium comprising an organic solvent, a BNNT, and a library molecule with an affinity for a BNNT of <1E−8 M.

A4.1.7.6: A phage-encoded peptide library, a medium comprising an organic solvent, a BNNT, and a library molecule with an affinity for a BNNT of <1E−9 M.

A4.1.7.7: A phage-encoded peptide library, a medium comprising an organic solvent, a BNNT, and a library molecule with an affinity for a BNNT of <1E−10 M.

A4.1.7.8: A phage-encoded peptide library, a medium comprising an organic solvent, a BNNT, and a library molecule with an affinity for a BNNT of <1E−12 M.

A4.2.1.1: A phage-encoded peptide library, a medium comprising an organic solvent and a soluble polymer, a CNT, and a library molecule with an affinity for a CNT of <1E−2 M.

A4.2.1.2: A phage-encoded peptide library, a medium comprising an organic solvent and a soluble polymer, a CNT, and a library molecule with an affinity for a CNT of <1E−4 M.

A4.2.1.3: A phage-encoded peptide library, a medium comprising an organic solvent and a soluble polymer, a CNT, and a library molecule with an affinity for a CNT of <1E−6 M.

A4.2.1.4: A phage-encoded peptide library, a medium comprising an organic solvent and a soluble polymer, a CNT, and a library molecule with an affinity for a CNT of <1E−7 M.

A4.2.1.5: A phage-encoded peptide library, a medium comprising an organic solvent and a soluble polymer, a CNT, and a library molecule with an affinity for a CNT of <1E−8 M.

A4.2.1.6: A phage-encoded peptide library, a medium comprising an organic solvent and a soluble polymer, a CNT, and a library molecule with an affinity for a CNT of <1E−9 M.

A4.2.1.7: A phage-encoded peptide library, a medium comprising an organic solvent and a soluble polymer, a CNT, and a library molecule with an affinity for a CNT of <1E−10 M.

A4.2.1.8: A phage-encoded peptide library, a medium comprising an organic solvent and a soluble polymer, a CNT, and a library molecule with an affinity for a CNT of <1E−12 M.

A4.2.2.1: A phage-encoded peptide library, a medium comprising an organic solvent and a soluble polymer, a GS, and a library molecule with an affinity for a GS of <1E−2 M.

A4.2.2.2: A phage-encoded peptide library, a medium comprising an organic solvent and a soluble polymer, a GS, and a library molecule with an affinity for a GS of <1E−4 M.

A4.2.2.3: A phage-encoded peptide library, a medium comprising an organic solvent and a soluble polymer, a GS, and a library molecule with an affinity for a GS of <1E−6 M.

A4.2.2.4: A phage-encoded peptide library, a medium comprising an organic solvent and a soluble polymer, a GS, and a library molecule with an affinity for a GS of <1E−7 M.

A4.2.2.5: A phage-encoded peptide library, a medium comprising an organic solvent and a soluble polymer, a GS, and a library molecule with an affinity for a GS of <1E−8 M.

A4.2.2.6: A phage-encoded peptide library, a medium comprising an organic solvent and a soluble polymer, a GS, and a library molecule with an affinity for a GS of <1E−9 M.

A4.2.2.7: A phage-encoded peptide library, a medium comprising an organic solvent and a soluble polymer, a GS, and a library molecule with an affinity for a GS of <1E−10 M.

A4.2.2.8: A phage-encoded peptide library, a medium comprising an organic solvent and a soluble polymer, a GS, and a library molecule with an affinity for a GS of <1E−12 M.

A4.2.3.1: A phage-encoded peptide library, a medium comprising an organic solvent and a soluble polymer, a SWNT, and a library molecule with an affinity for a SWNT of <1E−2 M.

A4.2.3.2: A phage-encoded peptide library, a medium comprising an organic solvent and a soluble polymer, a SWNT, and a library molecule with an affinity for a SWNT of <1E−4 M.

A4.2.3.3: A phage-encoded peptide library, a medium comprising an organic solvent and a soluble polymer, a SWNT, and a library molecule with an affinity for a SWNT of <1E−6 M.

A4.2.3.4: A phage-encoded peptide library, a medium comprising an organic solvent and a soluble polymer, a SWNT, and a library molecule with an affinity for a SWNT of <1E−7 M.

A4.2.3.5: A phage-encoded peptide library, a medium comprising an organic solvent and a soluble polymer, a SWNT, and a library molecule with an affinity for a SWNT of <1E−8 M.

A4.2.3.6: A phage-encoded peptide library, a medium comprising an organic solvent and a soluble polymer, a SWNT, and a library molecule with an affinity for a SWNT of <1E−9 M.

A4.2.3.7: A phage-encoded peptide library, a medium comprising an organic solvent and a soluble polymer, a SWNT, and a library molecule with an affinity for a SWNT of <1E−10 M.

A4.2.3.8: A phage-encoded peptide library, a medium comprising an organic solvent and a soluble polymer, a SWNT, and a library molecule with an affinity for a SWNT of <1E−12 M.

A4.2.4.1: A phage-encoded peptide library, a medium comprising an organic solvent and a soluble polymer, a MWNT, and a library molecule with an affinity for a MWNT of <1E−2 M.

A4.2.4.2: A phage-encoded peptide library, a medium comprising an organic solvent and a soluble polymer, a MWNT, and a library molecule with an affinity for a MWNT of <1E−4 M.

A4.2.4.3: A phage-encoded peptide library, a medium comprising an organic solvent and a soluble polymer, a MWNT, and a library molecule with an affinity for a MWNT of <1E−6 M.

A4.2.4.4: A phage-encoded peptide library, a medium comprising an organic solvent and a soluble polymer, a MWNT, and a library molecule with an affinity for a MWNT of <1E−7 M.

A4.2.4.5: A phage-encoded peptide library, a medium comprising an organic solvent and a soluble polymer, a MWNT, and a library molecule with an affinity for a MWNT of <1E−8 M.

A4.2.4.6: A phage-encoded peptide library, a medium comprising an organic solvent and a soluble polymer, a MWNT, and a library molecule with an affinity for a MWNT of <1E−9 M.

A4.2.4.7: A phage-encoded peptide library, a medium comprising an organic solvent and a soluble polymer, a MWNT, and a library molecule with an affinity for a MWNT of <1E−10 M.

A4.2.4.8: A phage-encoded peptide library, a medium comprising an organic solvent and a soluble polymer, a MWNT, and a library molecule with an affinity for a MWNT of <1E−12 M.

A4.2.5.1: A phage-encoded peptide library, a medium comprising an organic solvent and a soluble polymer, a nanotube, and a library molecule with an affinity for a nanotube of <1E−2 M.

A4.2.5.2: A phage-encoded peptide library, a medium comprising an organic solvent and a soluble polymer, a nanotube, and a library molecule with an affinity for a nanotube of <1E−4 M.

A4.2.5.3: A phage-encoded peptide library, a medium comprising an organic solvent and a soluble polymer, a nanotube, and a library molecule with an affinity for a nanotube of <1E−6 M.

A4.2.5.4: A phage-encoded peptide library, a medium comprising an organic solvent and a soluble polymer, a nanotube, and a library molecule with an affinity for a nanotube of <1E−7 M.

A4.2.5.5: A phage-encoded peptide library, a medium comprising an organic solvent and a soluble polymer, a nanotube, and a library molecule with an affinity for a nanotube of <1E−8 M.

A4.2.5.6: A phage-encoded peptide library, a medium comprising an organic solvent and a soluble polymer, a nanotube, and a library molecule with an affinity for a nanotube of <1E−9 M.

A4.2.5.7: A phage-encoded peptide library, a medium comprising an organic solvent and a soluble polymer, a nanotube, and a library molecule with an affinity for a nanotube of <1E−10 M.

A4.2.5.8: A phage-encoded peptide library, a medium comprising an organic solvent and a soluble polymer, a nanotube, and a library molecule with an affinity for a nanotube of <1E−12 M.

A4.2.6.1: A phage-encoded peptide library, a medium comprising an organic solvent and a soluble polymer, a fullerene, and a library molecule with an affinity for a fullerene of <1E−2 M.

A4.2.6.2: A phage-encoded peptide library, a medium comprising an organic solvent and a soluble polymer, a fullerene, and a library molecule with an affinity for a fullerene of <1E−4 M.

A4.2.6.3: A phage-encoded peptide library, a medium comprising an organic solvent and a soluble polymer, a fullerene, and a library molecule with an affinity for a fullerene of <1E−6 M.

A4.2.6.4: A phage-encoded peptide library, a medium comprising an organic solvent and a soluble polymer, a fullerene, and a library molecule with an affinity for a fullerene of <1E−7 M.

A4.2.6.5: A phage-encoded peptide library, a medium comprising an organic solvent and a soluble polymer, a fullerene, and a library molecule with an affinity for a fullerene of <1E−8 M.

A4.2.6.6: A phage-encoded peptide library, a medium comprising an organic solvent and a soluble polymer, a fullerene, and a library molecule with an affinity for a fullerene of <1E−9 M.

A4.2.6.7: A phage-encoded peptide library, a medium comprising an organic solvent and a soluble polymer, a fullerene, and a library molecule with an affinity for a fullerene of <1E−10 M.

A4.2.6.8: A phage-encoded peptide library, a medium comprising an organic solvent and a soluble polymer, a fullerene, and a library molecule with an affinity for a fullerene of <1E−12 M.

A4.2.7.1: A phage-encoded peptide library, a medium comprising an organic solvent and a soluble polymer, a BNNT, and a library molecule with an affinity for a BNNT of <1E−2 M.

A4.2.7.2: A phage-encoded peptide library, a medium comprising an organic solvent and a soluble polymer, a BNNT, and a library molecule with an affinity for a BNNT of <1E−4 M.

A4.2.7.3: A phage-encoded peptide library, a medium comprising an organic solvent and a soluble polymer, a BNNT, and a library molecule with an affinity for a BNNT of <1E−6 M.

A4.2.7.4: A phage-encoded peptide library, a medium comprising an organic solvent and a soluble polymer, a BNNT, and a library molecule with an affinity for a BNNT of <1E−7 M.

A4.2.7.5: A phage-encoded peptide library, a medium comprising an organic solvent and a soluble polymer, a BNNT, and a library molecule with an affinity for a BNNT of <1E−8 M.

A4.2.7.6: A phage-encoded peptide library, a medium comprising an organic solvent and a soluble polymer, a BNNT, and a library molecule with an affinity for a BNNT of <1E−9 M.

A4.2.7.7: A phage-encoded peptide library, a medium comprising an organic solvent and a soluble polymer, a BNNT, and a library molecule with an affinity for a BNNT of <1E−10 M.

A4.2.7.8: A phage-encoded peptide library, a medium comprising an organic solvent and a soluble polymer, a BNNT, and a library molecule with an affinity for a BNNT of <1E−12 M.

A4.3.1.1: A phage-encoded peptide library, a medium comprising a soluble polymer, a CNT, and a library molecule with an affinity for a CNT of <1E−2 M.

A4.3.1.2: A phage-encoded peptide library, a medium comprising a soluble polymer, a CNT, and a library molecule with an affinity for a CNT of <1E−4 M.

A4.3.1.3: A phage-encoded peptide library, a medium comprising a soluble polymer, a CNT, and a library molecule with an affinity for a CNT of <1E−6 M.

A4.3.1.4: A phage-encoded peptide library, a medium comprising a soluble polymer, a CNT, and a library molecule with an affinity for a CNT of <1E−7 M.

A4.3.1.5: A phage-encoded peptide library, a medium comprising a soluble polymer, a CNT, and a library molecule with an affinity for a CNT of <1E−8 M.

A4.3.1.6: A phage-encoded peptide library, a medium comprising a soluble polymer, a CNT, and a library molecule with an affinity for a CNT of <1E−9 M.

A4.3.1.7: A phage-encoded peptide library, a medium comprising a soluble polymer, a CNT, and a library molecule with an affinity for a CNT of <1E−10 M.

A4.3.1.8: A phage-encoded peptide library, a medium comprising a soluble polymer, a CNT, and a library molecule with an affinity for a CNT of <1E−12 M.

A4.3.2.1: A phage-encoded peptide library, a medium comprising a soluble polymer, a GS, and a library molecule with an affinity for a GS of <1E−2 M.

A4.3.2.2: A phage-encoded peptide library, a medium comprising a soluble polymer, a GS, and a library molecule with an affinity for a GS of <1E−4 M.

A4.3.2.3: A phage-encoded peptide library, a medium comprising a soluble polymer, a GS, and a library molecule with an affinity for a GS of <1E−6 M.

A4.3.2.4: A phage-encoded peptide library, a medium comprising a soluble polymer, a GS, and a library molecule with an affinity for a GS of <1E−7 M.

A4.3.2.5: A phage-encoded peptide library, a medium comprising a soluble polymer, a GS, and a library molecule with an affinity for a GS of <1E−8 M.

A4.3.2.6: A phage-encoded peptide library, a medium comprising a soluble polymer, a GS, and a library molecule with an affinity for a GS of <1E−9 M.

A4.3.2.7: A phage-encoded peptide library, a medium comprising a soluble polymer, a GS, and a library molecule with an affinity for a GS of <1E−10 M.

A4.3.2.8: A phage-encoded peptide library, a medium comprising a soluble polymer, a GS, and a library molecule with an affinity for a GS of <1E−12 M.

A4.3.3.1: A phage-encoded peptide library, a medium comprising a soluble polymer, a SWNT, and a library molecule with an affinity for a SWNT of <1E−2 M.

A4.3.3.2: A phage-encoded peptide library, a medium comprising a soluble polymer, a SWNT, and a library molecule with an affinity for a SWNT of <1E−4 M.

A4.3.3.3: A phage-encoded peptide library, a medium comprising a soluble polymer, a SWNT, and a library molecule with an affinity for a SWNT of <1E−6 M.

A4.3.3.4: A phage-encoded peptide library, a medium comprising a soluble polymer, a SWNT, and a library molecule with an affinity for a SWNT of <1E−7 M.

A4.3.3.5: A phage-encoded peptide library, a medium comprising a soluble polymer, a SWNT, and a library molecule with an affinity for a SWNT of <1E−8 M.

A4.3.3.6: A phage-encoded peptide library, a medium comprising a soluble polymer, a SWNT, and a library molecule with an affinity for a SWNT of <1E−9 M.

A4.3.3.7: A phage-encoded peptide library, a medium comprising a soluble polymer, a SWNT, and a library molecule with an affinity for a SWNT of <1E−10 M.

A4.3.3.8: A phage-encoded peptide library, a medium comprising a soluble polymer, a SWNT, and a library molecule with an affinity for a SWNT of <1E−12 M.

A4.3.4.1: A phage-encoded peptide library, a medium comprising a soluble polymer, a MWNT, and a library molecule with an affinity for a MWNT of <1E−2 M.

A4.3.4.2: A phage-encoded peptide library, a medium comprising a soluble polymer, a MWNT, and a library molecule with an affinity for a MWNT of <1E−4 M.

A4.3.4.3: A phage-encoded peptide library, a medium comprising a soluble polymer, a MWNT, and a library molecule with an affinity for a MWNT of <1E−6 M.

A4.3.4.4: A phage-encoded peptide library, a medium comprising a soluble polymer, a MWNT, and a library molecule with an affinity for a MWNT of <1E−7 M.

A4.3.4.5: A phage-encoded peptide library, a medium comprising a soluble polymer, a MWNT, and a library molecule with an affinity for a MWNT of <1E−8 M.

A4.3.4.6: A phage-encoded peptide library, a medium comprising a soluble polymer, a MWNT, and a library molecule with an affinity for a MWNT of <1E−9 M.

A4.3.4.7: A phage-encoded peptide library, a medium comprising a soluble polymer, a MWNT, and a library molecule with an affinity for a MWNT of <1E−10 M.

A4.3.4.8: A phage-encoded peptide library, a medium comprising a soluble polymer, a MWNT, and a library molecule with an affinity for a MWNT of <1E−12 M.

A4.3.5.1: A phage-encoded peptide library, a medium comprising a soluble polymer, a nanotube, and a library molecule with an affinity for a nanotube of <1E−2 M.

A4.3.5.2: A phage-encoded peptide library, a medium comprising a soluble polymer, a nanotube, and a library molecule with an affinity for a nanotube of <1E−4 M.

A4.3.5.3: A phage-encoded peptide library, a medium comprising a soluble polymer, a nanotube, and a library molecule with an affinity for a nanotube of <1E−6 M.

A4.3.5.4: A phage-encoded peptide library, a medium comprising a soluble polymer, a nanotube, and a library molecule with an affinity for a nanotube of <1E−7 M.

A4.3.5.5: A phage-encoded peptide library, a medium comprising a soluble polymer, a nanotube, and a library molecule with an affinity for a nanotube of <1E−8 M.

A4.3.5.6: A phage-encoded peptide library, a medium comprising a soluble polymer, a nanotube, and a library molecule with an affinity for a nanotube of <1E−9 M.

A4.3.5.7: A phage-encoded peptide library, a medium comprising a soluble polymer, a nanotube, and a library molecule with an affinity for a nanotube of <1E−10 M.

A4.3.5.8: A phage-encoded peptide library, a medium comprising a soluble polymer, a nanotube, and a library molecule with an affinity for a nanotube of <1E−12 M.

A4.3.6.1: A phage-encoded peptide library, a medium comprising a soluble polymer, a fullerene, and a library molecule with an affinity for a fullerene of <1E−2 M.

A4.3.6.2: A phage-encoded peptide library, a medium comprising a soluble polymer, a fullerene, and a library molecule with an affinity for a fullerene of <1E−4 M.

A4.3.6.3: A phage-encoded peptide library, a medium comprising a soluble polymer, a fullerene, and a library molecule with an affinity for a fullerene of <1E−6 M.

A4.3.6.4: A phage-encoded peptide library, a medium comprising a soluble polymer, a fullerene, and a library molecule with an affinity for a fullerene of <1E−7 M.

A4.3.6.5: A phage-encoded peptide library, a medium comprising a soluble polymer, a fullerene, and a library molecule with an affinity for a fullerene of <1E−8 M.

A4.3.6.6: A phage-encoded peptide library, a medium comprising a soluble polymer, a fullerene, and a library molecule with an affinity for a fullerene of <1E−9 M.

A4.3.6.7: A phage-encoded peptide library, a medium comprising a soluble polymer, a fullerene, and a library molecule with an affinity for a fullerene of <1E−10 M.

A4.3.6.8: A phage-encoded peptide library, a medium comprising a soluble polymer, a fullerene, and a library molecule with an affinity for a fullerene of <1E−12 M.

A4.3.7.1: A phage-encoded peptide library, a medium comprising a soluble polymer, a BNNT, and a library molecule with an affinity for a BNNT of <1E−2 M.

A4.3.7.2: A phage-encoded peptide library, a medium comprising a soluble polymer, a BNNT, and a library molecule with an affinity for a BNNT of <1E−4 M.

A4.3.7.3: A phage-encoded peptide library, a medium comprising a soluble polymer, a BNNT, and a library molecule with an affinity for a BNNT of <1E−6 M.

A4.3.7.4: A phage-encoded peptide library, a medium comprising a soluble polymer, a BNNT, and a library molecule with an affinity for a BNNT of <1E−7 M.

A4.3.7.5: A phage-encoded peptide library, a medium comprising a soluble polymer, a BNNT, and a library molecule with an affinity for a BNNT of <1E−8 M.

A4.3.7.6: A phage-encoded peptide library, a medium comprising a soluble polymer, a BNNT, and a library molecule with an affinity for a BNNT of <1E−9 M.

A4.3.7.7: A phage-encoded peptide library, a medium comprising a soluble polymer, a BNNT, and a library molecule with an affinity for a BNNT of <1E−10 M.

A4.3.7.8: A phage-encoded peptide library, a medium comprising a soluble polymer, a BNNT, and a library molecule with an affinity for a BNNT of <1E−12 M.

A4.4.1.1: A phage-encoded peptide library, a medium comprising >50% organic solvent, a CNT, and a library molecule with an affinity for a CNT of <1E−2 M.

A4.4.1.2: A phage-encoded peptide library, a medium comprising >50% organic solvent, a CNT, and a library molecule with an affinity for a CNT of <1E−4 M.

A4.4.1.3: A phage-encoded peptide library, a medium comprising >50% organic solvent, a CNT, and a library molecule with an affinity for a CNT of <1E−6 M.

A4.4.1.4: A phage-encoded peptide library, a medium comprising >50% organic solvent, a CNT, and a library molecule with an affinity for a CNT of <1E−7 M.

A4.4.1.5: A phage-encoded peptide library, a medium comprising >50% organic solvent, a CNT, and a library molecule with an affinity for a CNT of <1E−8 M.

A4.4.1.6: A phage-encoded peptide library, a medium comprising >50% organic solvent, a CNT, and a library molecule with an affinity for a CNT of <1E−9 M.

A4.4.1.7: A phage-encoded peptide library, a medium comprising >50% organic solvent, a CNT, and a library molecule with an affinity for a CNT of <1E−10 M.

A4.4.1.8: A phage-encoded peptide library, a medium comprising >50% organic solvent, a CNT, and a library molecule with an affinity for a CNT of <1E−12 M.

A4.4.2.1: A phage-encoded peptide library, a medium comprising >50% organic solvent, a GS, and a library molecule with an affinity for a GS of <1E−2 M.

A4.4.2.2: A phage-encoded peptide library, a medium comprising >50% organic solvent, a GS, and a library molecule with an affinity for a GS of <1E−4 M.

A4.4.2.3: A phage-encoded peptide library, a medium comprising >50% organic solvent, a GS, and a library molecule with an affinity for a GS of <1E−6 M.

A4.4.2.4: A phage-encoded peptide library, a medium comprising >50% organic solvent, a GS, and a library molecule with an affinity for a GS of <1E−7 M.

A4.4.2.5: A phage-encoded peptide library, a medium comprising >50% organic solvent, a GS, and a library molecule with an affinity for a GS of <1E−8 M.

A4.4.2.6: A phage-encoded peptide library, a medium comprising >50% organic solvent, a GS, and a library molecule with an affinity for a GS of <1E−9 M.

A4.4.2.7: A phage-encoded peptide library, a medium comprising >50% organic solvent, a GS, and a library molecule with an affinity for a GS of <1E−10 M.

A4.4.2.8: A phage-encoded peptide library, a medium comprising >50% organic solvent, a GS, and a library molecule with an affinity for a GS of <1E−12 M.

A4.4.3.1: A phage-encoded peptide library, a medium comprising >50% organic solvent, a SWNT, and a library molecule with an affinity for a SWNT of <1E−2 M.

A4.4.3.2: A phage-encoded peptide library, a medium comprising >50% organic solvent, a SWNT, and a library molecule with an affinity for a SWNT of <1E−4 M.

A4.4.3.3: A phage-encoded peptide library, a medium comprising >50% organic solvent, a SWNT, and a library molecule with an affinity for a SWNT of <1E−6 M.

A4.4.3.4: A phage-encoded peptide library, a medium comprising >50% organic solvent, a SWNT, and a library molecule with an affinity for a SWNT of <1E−7 M.

A4.4.3.5: A phage-encoded peptide library, a medium comprising >50% organic solvent, a SWNT, and a library molecule with an affinity for a SWNT of <1E−8 M.

A4.4.3.6: A phage-encoded peptide library, a medium comprising >50% organic solvent, a SWNT, and a library molecule with an affinity for a SWNT of <1E−9 M.

A4.4.3.7: A phage-encoded peptide library, a medium comprising >50% organic solvent, a SWNT, and a library molecule with an affinity for a SWNT of <1E−10 M.

A4.4.3.8: A phage-encoded peptide library, a medium comprising >50% organic solvent, a SWNT, and a library molecule with an affinity for a SWNT of <1E−12 M.

A4.4.4.1: A phage-encoded peptide library, a medium comprising >50% organic solvent, a MWNT, and a library molecule with an affinity for a MWNT of <1E−2 M.

A4.4.4.2: A phage-encoded peptide library, a medium comprising >50% organic solvent, a MWNT, and a library molecule with an affinity for a MWNT of <1E−4 M.

A4.4.4.3: A phage-encoded peptide library, a medium comprising >50% organic solvent, a MWNT, and a library molecule with an affinity for a MWNT of <1E−6 M.

A4.4.4.4: A phage-encoded peptide library, a medium comprising >50% organic solvent, a MWNT, and a library molecule with an affinity for a MWNT of <1E−7 M.

A4.4.4.5: A phage-encoded peptide library, a medium comprising >50% organic solvent, a MWNT, and a library molecule with an affinity for a MWNT of <1E−8 M.

A4.4.4.6: A phage-encoded peptide library, a medium comprising >50% organic solvent, a MWNT, and a library molecule with an affinity for a MWNT of <1E−9 M.

A4.4.4.7: A phage-encoded peptide library, a medium comprising >50% organic solvent, a MWNT, and a library molecule with an affinity for a MWNT of <1E−10 M.

A4.4.4.8: A phage-encoded peptide library, a medium comprising >50% organic solvent, a MWNT, and a library molecule with an affinity for a MWNT of <1E−12 M.

A4.4.5.1: A phage-encoded peptide library, a medium comprising >50% organic solvent, a nanotube, and a library molecule with an affinity for a nanotube of <1E−2 M.

A4.4.5.2: A phage-encoded peptide library, a medium comprising >50% organic solvent, a nanotube, and a library molecule with an affinity for a nanotube of <1E−4 M.

A4.4.5.3: A phage-encoded peptide library, a medium comprising >50% organic solvent, a nanotube, and a library molecule with an affinity for a nanotube of <1E−6 M.

A4.4.5.4: A phage-encoded peptide library, a medium comprising >50% organic solvent, a nanotube, and a library molecule with an affinity for a nanotube of <1E−7 M.

A4.4.5.5: A phage-encoded peptide library, a medium comprising >50% organic solvent, a nanotube, and a library molecule with an affinity for a nanotube of <1E−8 M.

A4.4.5.6: A phage-encoded peptide library, a medium comprising >50% organic solvent, a nanotube, and a library molecule with an affinity for a nanotube of <1E−9 M.

A4.4.5.7: A phage-encoded peptide library, a medium comprising >50% organic solvent, a nanotube, and a library molecule with an affinity for a nanotube of <1E−10 M.

A4.4.5.8: A phage-encoded peptide library, a medium comprising >50% organic solvent, a nanotube, and a library molecule with an affinity for a nanotube of <1E−12 M.

A4.4.6.1: A phage-encoded peptide library, a medium comprising >50% organic solvent, a fullerene, and a library molecule with an affinity for a fullerene of <1E−2 M.

A4.4.6.2: A phage-encoded peptide library, a medium comprising >50% organic solvent, a fullerene, and a library molecule with an affinity for a fullerene of <1E−4 M.

A4.4.6.3: A phage-encoded peptide library, a medium comprising >50% organic solvent, a fullerene, and a library molecule with an affinity for a fullerene of <1E−6 M.

A4.4.6.4: A phage-encoded peptide library, a medium comprising >50% organic solvent, a fullerene, and a library molecule with an affinity for a fullerene of <1E−7 M.

A4.4.6.5: A phage-encoded peptide library, a medium comprising >50% organic solvent, a fullerene, and a library molecule with an affinity for a fullerene of <1E−8 M.

A4.4.6.6: A phage-encoded peptide library, a medium comprising >50% organic solvent, a fullerene, and a library molecule with an affinity for a fullerene of <1E−9 M.

A4.4.6.7: A phage-encoded peptide library, a medium comprising >50% organic solvent, a fullerene, and a library molecule with an affinity for a fullerene of <1E−10 M.

A4.4.6.8: A phage-encoded peptide library, a medium comprising >50% organic solvent, a fullerene, and a library molecule with an affinity for a fullerene of <1E−12 M.

A4.4.7.1: A phage-encoded peptide library, a medium comprising >50% organic solvent, a BNNT, and a library molecule with an affinity for a BNNT of <1E−2 M.

A4.4.7.2: A phage-encoded peptide library, a medium comprising >50% organic solvent, a BNNT, and a library molecule with an affinity for a BNNT of <1E−4 M.

A4.4.7.3: A phage-encoded peptide library, a medium comprising >50% organic solvent, a BNNT, and a library molecule with an affinity for a BNNT of <1E−6 M.

A4.4.7.4: A phage-encoded peptide library, a medium comprising >50% organic solvent, a BNNT, and a library molecule with an affinity for a BNNT of <1E−7 M.

A4.4.7.5: A phage-encoded peptide library, a medium comprising >50% organic solvent, a BNNT, and a library molecule with an affinity for a BNNT of <1E−8 M.

A4.4.7.6: A phage-encoded peptide library, a medium comprising >50% organic solvent, a BNNT, and a library molecule with an affinity for a BNNT of <1E−9 M.

A4.4.7.7: A phage-encoded peptide library, a medium comprising >50% organic solvent, a BNNT, and a library molecule with an affinity for a BNNT of <1E−10 M.

A4.4.7.8: A phage-encoded peptide library, a medium comprising >50% organic solvent, a BNNT, and a library molecule with an affinity for a BNNT of <1E−12 M.

A4.5.1.1: A phage-encoded peptide library, a medium comprising >50% organic solvent and a soluble polymer, a CNT, and a library molecule with an affinity for a CNT of <1E−2 M.

A4.5.1.2: A phage-encoded peptide library, a medium comprising >50% organic solvent and a soluble polymer, a CNT, and a library molecule with an affinity for a CNT of <1E−4 M.

A4.5.1.3: A phage-encoded peptide library, a medium comprising >50% organic solvent and a soluble polymer, a CNT, and a library molecule with an affinity for a CNT of <1E−6 M.

A4.5.1.4: A phage-encoded peptide library, a medium comprising >50% organic solvent and a soluble polymer, a CNT, and a library molecule with an affinity for a CNT of <1E−7 M.

A4.5.1.5: A phage-encoded peptide library, a medium comprising >50% organic solvent and a soluble polymer, a CNT, and a library molecule with an affinity for a CNT of <1E−8 M.

A4.5.1.6: A phage-encoded peptide library, a medium comprising >50% organic solvent and a soluble polymer, a CNT, and a library molecule with an affinity for a CNT of <1E−9 M.

A4.5.1.7: A phage-encoded peptide library, a medium comprising >50% organic solvent and a soluble polymer, a CNT, and a library molecule with an affinity for a CNT of <1E−10 M.

A4.5.1.8: A phage-encoded peptide library, a medium comprising >50% organic solvent and a soluble polymer, a CNT, and a library molecule with an affinity for a CNT of <1E−12 M.

A4.5.2.1: A phage-encoded peptide library, a medium comprising >50% organic solvent and a soluble polymer, a GS, and a library molecule with an affinity for a GS of <1E−2 M.

A4.5.2.2: A phage-encoded peptide library, a medium comprising >50% organic solvent and a soluble polymer, a GS, and a library molecule with an affinity for a GS of <1E−4 M.

A4.5.2.3: A phage-encoded peptide library, a medium comprising >50% organic solvent and a soluble polymer, a GS, and a library molecule with an affinity for a GS of <1E−6 M.

A4.5.2.4: A phage-encoded peptide library, a medium comprising >50% organic solvent and a soluble polymer, a GS, and a library molecule with an affinity for a GS of <1E−7 M.

A4.5.2.5: A phage-encoded peptide library, a medium comprising >50% organic solvent and a soluble polymer, a GS, and a library molecule with an affinity for a GS of <1E−8 M.

A4.5.2.6: A phage-encoded peptide library, a medium comprising >50% organic solvent and a soluble polymer, a GS, and a library molecule with an affinity for a GS of <1E−9 M.

A4.5.2.7: A phage-encoded peptide library, a medium comprising >50% organic solvent and a soluble polymer, a GS, and a library molecule with an affinity for a GS of <1E−10 M.

A4.5.2.8: A phage-encoded peptide library, a medium comprising >50% organic solvent and a soluble polymer, a GS, and a library molecule with an affinity for a GS of <1E−12 M.

A4.5.3.1: A phage-encoded peptide library, a medium comprising >50% organic solvent and a soluble polymer, a SWNT, and a library molecule with an affinity for a SWNT of <1E−2 M.

A4.5.3.2: A phage-encoded peptide library, a medium comprising >50% organic solvent and a soluble polymer, a SWNT, and a library molecule with an affinity for a SWNT of <1E−4 M.

A4.5.3.3: A phage-encoded peptide library, a medium comprising >50% organic solvent and a soluble polymer, a SWNT, and a library molecule with an affinity for a SWNT of <1E−6 M.

A4.5.3.4: A phage-encoded peptide library, a medium comprising >50% organic solvent and a soluble polymer, a SWNT, and a library molecule with an affinity for a SWNT of <1E−7 M.

A4.5.3.5: A phage-encoded peptide library, a medium comprising >50% organic solvent and a soluble polymer, a SWNT, and a library molecule with an affinity for a SWNT of <1E−8 M.

A4.5.3.6: A phage-encoded peptide library, a medium comprising >50% organic solvent and a soluble polymer, a SWNT, and a library molecule with an affinity for a SWNT of <1E−9 M.

A4.5.3.7: A phage-encoded peptide library, a medium comprising >50% organic solvent and a soluble polymer, a SWNT, and a library molecule with an affinity for a SWNT of <1E−10 M.

A4.5.3.8: A phage-encoded peptide library, a medium comprising >50% organic solvent and a soluble polymer, a SWNT, and a library molecule with an affinity for a SWNT of <1E−12 M.

A4.5.4.1: A phage-encoded peptide library, a medium comprising >50% organic solvent and a soluble polymer, a MWNT, and a library molecule with an affinity for a MWNT of <1E−2 M.

A4.5.4.2: A phage-encoded peptide library, a medium comprising >50% organic solvent and a soluble polymer, a MWNT, and a library molecule with an affinity for a MWNT of <1E−4 M.

A4.5.4.3: A phage-encoded peptide library, a medium comprising >50% organic solvent and a soluble polymer, a MWNT, and a library molecule with an affinity for a MWNT of <1E−6 M.

A4.5.4.4: A phage-encoded peptide library, a medium comprising >50% organic solvent and a soluble polymer, a MWNT, and a library molecule with an affinity for a MWNT of <1E−7 M.

A4.5.4.5: A phage-encoded peptide library, a medium comprising >50% organic solvent and a soluble polymer, a MWNT, and a library molecule with an affinity for a MWNT of <1E−8 M.

A4.5.4.6: A phage-encoded peptide library, a medium comprising >50% organic solvent and a soluble polymer, a MWNT, and a library molecule with an affinity for a MWNT of <1E−9 M.

A4.5.4.7: A phage-encoded peptide library, a medium comprising >50% organic solvent and a soluble polymer, a MWNT, and a library molecule with an affinity for a MWNT of <1E−10 M.

A4.5.4.8: A phage-encoded peptide library, a medium comprising >50% organic solvent and a soluble polymer, a MWNT, and a library molecule with an affinity for a MWNT of <1E−12 M.

A4.5.5.1: A phage-encoded peptide library, a medium comprising >50% organic solvent and a soluble polymer, a nanotube, and a library molecule with an affinity for a nanotube of <1E−2 M.

A4.5.5.2: A phage-encoded peptide library, a medium comprising >50% organic solvent and a soluble polymer, a nanotube, and a library molecule with an affinity for a nanotube of <1E−4 M.

A4.5.5.3: A phage-encoded peptide library, a medium comprising >50% organic solvent and a soluble polymer, a nanotube, and a library molecule with an affinity for a nanotube of <1E−6 M.

A4.5.5.4: A phage-encoded peptide library, a medium comprising >50% organic solvent and a soluble polymer, a nanotube, and a library molecule with an affinity for a nanotube of <1E−7 M.

A4.5.5.5: A phage-encoded peptide library, a medium comprising >50% organic solvent and a soluble polymer, a nanotube, and a library molecule with an affinity for a nanotube of <1E−8 M.

A4.5.5.6: A phage-encoded peptide library, a medium comprising >50% organic solvent and a soluble polymer, a nanotube, and a library molecule with an affinity for a nanotube of <1E−9 M.

A4.5.5.7: A phage-encoded peptide library, a medium comprising >50% organic solvent and a soluble polymer, a nanotube, and a library molecule with an affinity for a nanotube of <1E−10 M.

A4.5.5.8: A phage-encoded peptide library, a medium comprising >50% organic solvent and a soluble polymer, a nanotube, and a library molecule with an affinity for a nanotube of <1E−12 M.

A4.5.6.1: A phage-encoded peptide library, a medium comprising >50% organic solvent and a soluble polymer, a fullerene, and a library molecule with an affinity for a fullerene of <1E−2 M.

A4.5.6.2: A phage-encoded peptide library, a medium comprising >50% organic solvent and a soluble polymer, a fullerene, and a library molecule with an affinity for a fullerene of <1E−4 M.

A4.5.6.3: A phage-encoded peptide library, a medium comprising >50% organic solvent and a soluble polymer, a fullerene, and a library molecule with an affinity for a fullerene of <1E−6 M.

A4.5.6.4: A phage-encoded peptide library, a medium comprising >50% organic solvent and a soluble polymer, a fullerene, and a library molecule with an affinity for a fullerene of <1E−7 M.

A4.5.6.5: A phage-encoded peptide library, a medium comprising >50% organic solvent and a soluble polymer, a fullerene, and a library molecule with an affinity for a fullerene of <1E−8 M.

A4.5.6.6: A phage-encoded peptide library, a medium comprising >50% organic solvent and a soluble polymer, a fullerene, and a library molecule with an affinity for a fullerene of <1E−9 M.

A4.5.6.7: A phage-encoded peptide library, a medium comprising >50% organic solvent and a soluble polymer, a fullerene, and a library molecule with an affinity for a fullerene of <1E−10 M.

A4.5.6.8: A phage-encoded peptide library, a medium comprising >50% organic solvent and a soluble polymer, a fullerene, and a library molecule with an affinity for a fullerene of <1E−12 M.

A4.5.7.1: A phage-encoded peptide library, a medium comprising >50% organic solvent and a soluble polymer, a BNNT, and a library molecule with an affinity for a BNNT of <1E−2 M.

A4.5.7.2: A phage-encoded peptide library, a medium comprising >50% organic solvent and a soluble polymer, a BNNT, and a library molecule with an affinity for a BNNT of <1E−4 M.

A4.5.7.3: A phage-encoded peptide library, a medium comprising >50% organic solvent and a soluble polymer, a BNNT, and a library molecule with an affinity for a BNNT of <1E−6 M.

A4.5.7.4: A phage-encoded peptide library, a medium comprising >50% organic solvent and a soluble polymer, a BNNT, and a library molecule with an affinity for a BNNT of <1E−7 M.

A4.5.7.5: A phage-encoded peptide library, a medium comprising >50% organic solvent and a soluble polymer, a BNNT, and a library molecule with an affinity for a BNNT of <1E−8 M.

A4.5.7.6: A phage-encoded peptide library, a medium comprising >50% organic solvent and a soluble polymer, a BNNT, and a library molecule with an affinity for a BNNT of <1E−9 M.

A4.5.7.7: A phage-encoded peptide library, a medium comprising >50% organic solvent and a soluble polymer, a BNNT, and a library molecule with an affinity for a BNNT of <1E−10 M.

A4.5.7.8: A phage-encoded peptide library, a medium comprising >50% organic solvent and a soluble polymer, a BNNT, and a library molecule with an affinity for a BNNT of <1E−12 M.

A4.6.1.1: A phage-encoded peptide library, a medium comprising >80% organic solvent, a CNT, and a library molecule with an affinity for a CNT of <1E−2 M.

A4.6.1.2: A phage-encoded peptide library, a medium comprising >80% organic solvent, a CNT, and a library molecule with an affinity for a CNT of <1E−4 M.

A4.6.1.3: A phage-encoded peptide library, a medium comprising >80% organic solvent, a CNT, and a library molecule with an affinity for a CNT of <1E−6 M.

A4.6.1.4: A phage-encoded peptide library, a medium comprising >80% organic solvent, a CNT, and a library molecule with an affinity for a CNT of <1E−7 M.

A4.6.1.5: A phage-encoded peptide library, a medium comprising >80% organic solvent, a CNT, and a library molecule with an affinity for a CNT of <1E−8 M.

A4.6.1.6: A phage-encoded peptide library, a medium comprising >80% organic solvent, a CNT, and a library molecule with an affinity for a CNT of <1E−9 M.

A4.6.1.7: A phage-encoded peptide library, a medium comprising >80% organic solvent, a CNT, and a library molecule with an affinity for a CNT of <1E−10 M.

A4.6.1.8: A phage-encoded peptide library, a medium comprising >80% organic solvent, a CNT, and a library molecule with an affinity for a CNT of <1E−12 M.

A4.6.2.1: A phage-encoded peptide library, a medium comprising >80% organic solvent, a GS, and a library molecule with an affinity for a GS of <1E−2 M.

A4.6.2.2: A phage-encoded peptide library, a medium comprising >80% organic solvent, a GS, and a library molecule with an affinity for a GS of <1E−4 M.

A4.6.2.3: A phage-encoded peptide library, a medium comprising >80% organic solvent, a GS, and a library molecule with an affinity for a GS of <1E−6 M.

A4.6.2.4: A phage-encoded peptide library, a medium comprising >80% organic solvent, a GS, and a library molecule with an affinity for a GS of <1E−7 M.

A4.6.2.5: A phage-encoded peptide library, a medium comprising >80% organic solvent, a GS, and a library molecule with an affinity for a GS of <1E−8 M.

A4.6.2.6: A phage-encoded peptide library, a medium comprising >80% organic solvent, a GS, and a library molecule with an affinity for a GS of <1E−9 M.

A4.6.2.7: A phage-encoded peptide library, a medium comprising >80% organic solvent, a GS, and a library molecule with an affinity for a GS of <1E−10 M.

A4.6.2.8: A phage-encoded peptide library, a medium comprising >80% organic solvent, a GS, and a library molecule with an affinity for a GS of <1E−12 M.

A4.6.3.1: A phage-encoded peptide library, a medium comprising >80% organic solvent, a SWNT, and a library molecule with an affinity for a SWNT of <1E−2 M.

A4.6.3.2: A phage-encoded peptide library, a medium comprising >80% organic solvent, a SWNT, and a library molecule with an affinity for a SWNT of <1E−4 M.

A4.6.3.3: A phage-encoded peptide library, a medium comprising >80% organic solvent, a SWNT, and a library molecule with an affinity for a SWNT of <1E−6 M.

A4.6.3.4: A phage-encoded peptide library, a medium comprising >80% organic solvent, a SWNT, and a library molecule with an affinity for a SWNT of <1E−7 M.

A4.6.3.5: A phage-encoded peptide library, a medium comprising >80% organic solvent, a SWNT, and a library molecule with an affinity for a SWNT of <1E−8 M.

A4.6.3.6: A phage-encoded peptide library, a medium comprising >80% organic solvent, a SWNT, and a library molecule with an affinity for a SWNT of <1E−9 M.

A4.6.3.7: A phage-encoded peptide library, a medium comprising >80% organic solvent, a SWNT, and a library molecule with an affinity for a SWNT of <1E−10 M.

A4.6.3.8: A phage-encoded peptide library, a medium comprising >80% organic solvent, a SWNT, and a library molecule with an affinity for a SWNT of <1E−12 M.

A4.6.4.1: A phage-encoded peptide library, a medium comprising >80% organic solvent, a MWNT, and a library molecule with an affinity for a MWNT of <1E−2 M.

A4.6.4.2: A phage-encoded peptide library, a medium comprising >80% organic solvent, a MWNT, and a library molecule with an affinity for a MWNT of <1E−4 M.

A4.6.4.3: A phage-encoded peptide library, a medium comprising >80% organic solvent, a MWNT, and a library molecule with an affinity for a MWNT of <1E−6 M.

A4.6.4.4: A phage-encoded peptide library, a medium comprising >80% organic solvent, a MWNT, and a library molecule with an affinity for a MWNT of <1E−7 M.

A4.6.4.5: A phage-encoded peptide library, a medium comprising >80% organic solvent, a MWNT, and a library molecule with an affinity for a MWNT of <1E−8 M.

A4.6.4.6: A phage-encoded peptide library, a medium comprising >80% organic solvent, a MWNT, and a library molecule with an affinity for a MWNT of <1E−9 M.

A4.6.4.7: A phage-encoded peptide library, a medium comprising >80% organic solvent, a MWNT, and a library molecule with an affinity for a MWNT of <1E−10 M.

A4.6.4.8: A phage-encoded peptide library, a medium comprising >80% organic solvent, a MWNT, and a library molecule with an affinity for a MWNT of <1E−12 M.

A4.6.5.1: A phage-encoded peptide library, a medium comprising >80% organic solvent, a nanotube, and a library molecule with an affinity for a nanotube of <1E−2 M.

A4.6.5.2: A phage-encoded peptide library, a medium comprising >80% organic solvent, a nanotube, and a library molecule with an affinity for a nanotube of <1E−4 M.

A4.6.5.3: A phage-encoded peptide library, a medium comprising >80% organic solvent, a nanotube, and a library molecule with an affinity for a nanotube of <1E−6 M.

A4.6.5.4: A phage-encoded peptide library, a medium comprising >80% organic solvent, a nanotube, and a library molecule with an affinity for a nanotube of <1E−7 M.

A4.6.5.5: A phage-encoded peptide library, a medium comprising >80% organic solvent, a nanotube, and a library molecule with an affinity for a nanotube of <1E−8 M.

A4.6.5.6: A phage-encoded peptide library, a medium comprising >80% organic solvent, a nanotube, and a library molecule with an affinity for a nanotube of <1E−9 M.

A4.6.5.7: A phage-encoded peptide library, a medium comprising >80% organic solvent, a nanotube, and a library molecule with an affinity for a nanotube of <1E−10 M.

A4.6.5.8: A phage-encoded peptide library, a medium comprising >80% organic solvent, a nanotube, and a library molecule with an affinity for a nanotube of <1E−12 M.

A4.6.6.1: A phage-encoded peptide library, a medium comprising >80% organic solvent, a fullerene, and a library molecule with an affinity for a fullerene of <1E−2 M.

A4.6.6.2: A phage-encoded peptide library, a medium comprising >80% organic solvent, a fullerene, and a library molecule with an affinity for a fullerene of <1E−4 M.

A4.6.6.3: A phage-encoded peptide library, a medium comprising >80% organic solvent, a fullerene, and a library molecule with an affinity for a fullerene of <1E−6 M.

A4.6.6.4: A phage-encoded peptide library, a medium comprising >80% organic solvent, a fullerene, and a library molecule with an affinity for a fullerene of <1E−7 M.

A4.6.6.5: A phage-encoded peptide library, a medium comprising >80% organic solvent, a fullerene, and a library molecule with an affinity for a fullerene of <1E−8 M.

A4.6.6.6: A phage-encoded peptide library, a medium comprising >80% organic solvent, a fullerene, and a library molecule with an affinity for a fullerene of <1E−9 M.

A4.6.6.7: A phage-encoded peptide library, a medium comprising >80% organic solvent, a fullerene, and a library molecule with an affinity for a fullerene of <1E−10 M.

A4.6.6.8: A phage-encoded peptide library, a medium comprising >80% organic solvent, a fullerene, and a library molecule with an affinity for a fullerene of <1E−12 M.

A4.6.7.1: A phage-encoded peptide library, a medium comprising >80% organic solvent, a BNNT, and a library molecule with an affinity for a BNNT of <1E−2 M.

A4.6.7.2: A phage-encoded peptide library, a medium comprising >80% organic solvent, a BNNT, and a library molecule with an affinity for a BNNT of <1E−4 M.

A4.6.7.3: A phage-encoded peptide library, a medium comprising >80% organic solvent, a BNNT, and a library molecule with an affinity for a BNNT of <1E−6 M.

A4.6.7.4: A phage-encoded peptide library, a medium comprising >80% organic solvent, a BNNT, and a library molecule with an affinity for a BNNT of <1E−7 M.

A4.6.7.5: A phage-encoded peptide library, a medium comprising >80% organic solvent, a BNNT, and a library molecule with an affinity for a BNNT of <1E−8 M.

A4.6.7.6: A phage-encoded peptide library, a medium comprising >80% organic solvent, a BNNT, and a library molecule with an affinity for a BNNT of <1E−9 M.

A4.6.7.7: A phage-encoded peptide library, a medium comprising >80% organic solvent, a BNNT, and a library molecule with an affinity for a BNNT of <1E−10 M.

A4.6.7.8: A phage-encoded peptide library, a medium comprising >80% organic solvent, a BNNT, and a library molecule with an affinity for a BNNT of <1E−12 M.

A4.7.1.1: A phage-encoded peptide library, a medium comprising >80% organic solvent and a soluble polymer, a CNT, and a library molecule with an affinity for a CNT of <1E−2 M.

A4.7.1.2: A phage-encoded peptide library, a medium comprising >80% organic solvent and a soluble polymer, a CNT, and a library molecule with an affinity for a CNT of <1E−4 M.

A4.7.1.3: A phage-encoded peptide library, a medium comprising >80% organic solvent and a soluble polymer, a CNT, and a library molecule with an affinity for a CNT of <1E−6 M.

A4.7.1.4: A phage-encoded peptide library, a medium comprising >80% organic solvent and a soluble polymer, a CNT, and a library molecule with an affinity for a CNT of <1E−7 M.

A4.7.1.5: A phage-encoded peptide library, a medium comprising >80% organic solvent and a soluble polymer, a CNT, and a library molecule with an affinity for a CNT of <1E−8 M.

A4.7.1.6: A phage-encoded peptide library, a medium comprising >80% organic solvent and a soluble polymer, a CNT, and a library molecule with an affinity for a CNT of <1E−9 M.

A4.7.1.7: A phage-encoded peptide library, a medium comprising >80% organic solvent and a soluble polymer, a CNT, and a library molecule with an affinity for a CNT of <1E−10 M.

A4.7.1.8: A phage-encoded peptide library, a medium comprising >80% organic solvent and a soluble polymer, a CNT, and a library molecule with an affinity for a CNT of <1E−12 M.

A4.7.2.1: A phage-encoded peptide library, a medium comprising >80% organic solvent and a soluble polymer, a GS, and a library molecule with an affinity for a GS of <1E−2 M.

A4.7.2.2: A phage-encoded peptide library, a medium comprising >80% organic solvent and a soluble polymer, a GS, and a library molecule with an affinity for a GS of <1E−4 M.

A4.7.2.3: A phage-encoded peptide library, a medium comprising >80% organic solvent and a soluble polymer, a GS, and a library molecule with an affinity for a GS of <1E−6 M.

A4.7.2.4: A phage-encoded peptide library, a medium comprising >80% organic solvent and a soluble polymer, a GS, and a library molecule with an affinity for a GS of <1E−7 M.

A4.7.2.5: A phage-encoded peptide library, a medium comprising >80% organic solvent and a soluble polymer, a GS, and a library molecule with an affinity for a GS of <1E−8 M.

A4.7.2.6: A phage-encoded peptide library, a medium comprising >80% organic solvent and a soluble polymer, a GS, and a library molecule with an affinity for a GS of <1E−9 M.

A4.7.2.7: A phage-encoded peptide library, a medium comprising >80% organic solvent and a soluble polymer, a GS, and a library molecule with an affinity for a GS of <1E−10 M.

A4.7.2.8: A phage-encoded peptide library, a medium comprising >80% organic solvent and a soluble polymer, a GS, and a library molecule with an affinity for a GS of <1E−12 M.

A4.7.3.1: A phage-encoded peptide library, a medium comprising >80% organic solvent and a soluble polymer, a SWNT, and a library molecule with an affinity for a SWNT of <1E−2 M.

A4.7.3.2: A phage-encoded peptide library, a medium comprising >80% organic solvent and a soluble polymer, a SWNT, and a library molecule with an affinity for a SWNT of <1E−4 M.

A4.7.3.3: A phage-encoded peptide library, a medium comprising >80% organic solvent and a soluble polymer, a SWNT, and a library molecule with an affinity for a SWNT of <1E−6 M.

A4.7.3.4: A phage-encoded peptide library, a medium comprising >80% organic solvent and a soluble polymer, a SWNT, and a library molecule with an affinity for a SWNT of <1E−7 M.

A4.7.3.5: A phage-encoded peptide library, a medium comprising >80% organic solvent and a soluble polymer, a SWNT, and a library molecule with an affinity for a SWNT of <1E−8 M.

A4.7.3.6: A phage-encoded peptide library, a medium comprising >80% organic solvent and a soluble polymer, a SWNT, and a library molecule with an affinity for a SWNT of <1E−9 M.

A4.7.3.7: A phage-encoded peptide library, a medium comprising >80% organic solvent and a soluble polymer, a SWNT, and a library molecule with an affinity for a SWNT of <1E−10 M.

A4.7.3.8: A phage-encoded peptide library, a medium comprising >80% organic solvent and a soluble polymer, a SWNT, and a library molecule with an affinity for a SWNT of <1E−12 M.

A4.7.4.1: A phage-encoded peptide library, a medium comprising >80% organic solvent and a soluble polymer, a MWNT, and a library molecule with an affinity for a MWNT of <1E−2 M.

A4.7.4.2: A phage-encoded peptide library, a medium comprising >80% organic solvent and a soluble polymer, a MWNT, and a library molecule with an affinity for a MWNT of <1E−4 M.

A4.7.4.3: A phage-encoded peptide library, a medium comprising >80% organic solvent and a soluble polymer, a MWNT, and a library molecule with an affinity for a MWNT of <1E−6 M.

A4.7.4.4: A phage-encoded peptide library, a medium comprising >80% organic solvent and a soluble polymer, a MWNT, and a library molecule with an affinity for a MWNT of <1E−7 M.

A4.7.4.5: A phage-encoded peptide library, a medium comprising >80% organic solvent and a soluble polymer, a MWNT, and a library molecule with an affinity for a MWNT of <1E−8 M.

A4.7.4.6: A phage-encoded peptide library, a medium comprising >80% organic solvent and a soluble polymer, a MWNT, and a library molecule with an affinity for a MWNT of <1E−9 M.

A4.7.4.7: A phage-encoded peptide library, a medium comprising >80% organic solvent and a soluble polymer, a MWNT, and a library molecule with an affinity for a MWNT of <1E−10 M.

A4.7.4.8: A phage-encoded peptide library, a medium comprising >80% organic solvent and a soluble polymer, a MWNT, and a library molecule with an affinity for a MWNT of <1E−12 M.

A4.7.5.1: A phage-encoded peptide library, a medium comprising >80% organic solvent and a soluble polymer, a nanotube, and a library molecule with an affinity for a nanotube of <1E−2 M.

A4.7.5.2: A phage-encoded peptide library, a medium comprising >80% organic solvent and a soluble polymer, a nanotube, and a library molecule with an affinity for a nanotube of <1E−4 M.

A4.7.5.3: A phage-encoded peptide library, a medium comprising >80% organic solvent and a soluble polymer, a nanotube, and a library molecule with an affinity for a nanotube of <1E−6 M.

A4.7.5.4: A phage-encoded peptide library, a medium comprising >80% organic solvent and a soluble polymer, a nanotube, and a library molecule with an affinity for a nanotube of <1E−7 M.

A4.7.5.5: A phage-encoded peptide library, a medium comprising >80% organic solvent and a soluble polymer, a nanotube, and a library molecule with an affinity for a nanotube of <1E−8 M.

A4.7.5.6: A phage-encoded peptide library, a medium comprising >80% organic solvent and a soluble polymer, a nanotube, and a library molecule with an affinity for a nanotube of <1E−9 M.

A4.7.5.7: A phage-encoded peptide library, a medium comprising >80% organic solvent and a soluble polymer, a nanotube, and a library molecule with an affinity for a nanotube of <1E−10 M.

A4.7.5.8: A phage-encoded peptide library, a medium comprising >80% organic solvent and a soluble polymer, a nanotube, and a library molecule with an affinity for a nanotube of <1E−12 M.

A4.7.6.1: A phage-encoded peptide library, a medium comprising >80% organic solvent and a soluble polymer, a fullerene, and a library molecule with an affinity for a fullerene of <1E−2 M.

A4.7.6.2: A phage-encoded peptide library, a medium comprising >80% organic solvent and a soluble polymer, a fullerene, and a library molecule with an affinity for a fullerene of <1E−4 M.

A4.7.6.3: A phage-encoded peptide library, a medium comprising >80% organic solvent and a soluble polymer, a fullerene, and a library molecule with an affinity for a fullerene of <1E−6 M.

A4.7.6.4: A phage-encoded peptide library, a medium comprising >80% organic solvent and a soluble polymer, a fullerene, and a library molecule with an affinity for a fullerene of <1E−7 M.

A4.7.6.5: A phage-encoded peptide library, a medium comprising >80% organic solvent and a soluble polymer, a fullerene, and a library molecule with an affinity for a fullerene of <1E−8 M.

A4.7.6.6: A phage-encoded peptide library, a medium comprising >80% organic solvent and a soluble polymer, a fullerene, and a library molecule with an affinity for a fullerene of <1E−9 M.

A4.7.6.7: A phage-encoded peptide library, a medium comprising >80% organic solvent and a soluble polymer, a fullerene, and a library molecule with an affinity for a fullerene of <1E−10 M.

A4.7.6.8: A phage-encoded peptide library, a medium comprising >80% organic solvent and a soluble polymer, a fullerene, and a library molecule with an affinity for a fullerene of <1E−12 M.

A4.7.7.1: A phage-encoded peptide library, a medium comprising >80% organic solvent and a soluble polymer, a BNNT, and a library molecule with an affinity for a BNNT of <1E−2 M.

A4.7.7.2: A phage-encoded peptide library, a medium comprising >80% organic solvent and a soluble polymer, a BNNT, and a library molecule with an affinity for a BNNT of <1E−4 M.

A4.7.7.3: A phage-encoded peptide library, a medium comprising >80% organic solvent and a soluble polymer, a BNNT, and a library molecule with an affinity for a BNNT of <1E−6 M.

A4.7.7.4: A phage-encoded peptide library, a medium comprising >80% organic solvent and a soluble polymer, a BNNT, and a library molecule with an affinity for a BNNT of <1E−7 M.

A4.7.7.5: A phage-encoded peptide library, a medium comprising >80% organic solvent and a soluble polymer, a BNNT, and a library molecule with an affinity for a BNNT of <1E−8 M.

A4.7.7.6: A phage-encoded peptide library, a medium comprising >80% organic solvent and a soluble polymer, a BNNT, and a library molecule with an affinity for a BNNT of <1E−9 M.

A4.7.7.7: A phage-encoded peptide library, a medium comprising >80% organic solvent and a soluble polymer, a BNNT, and a library molecule with an affinity for a BNNT of <1E−10 M.

A4.7.7.8: A phage-encoded peptide library, a medium comprising >80% organic solvent and a soluble polymer, a BNNT, and a library molecule with an affinity for a BNNT of <1E−12 M.

A4.8.1.1: A phage-encoded peptide library, a medium comprising >95% organic solvent, a CNT, and a library molecule with an affinity for a CNT of <1E−2 M.

A4.8.1.2: A phage-encoded peptide library, a medium comprising >95% organic solvent, a CNT, and a library molecule with an affinity for a CNT of <1E−4 M.

A4.8.1.3: A phage-encoded peptide library, a medium comprising >95% organic solvent, a CNT, and a library molecule with an affinity for a CNT of <1E−6 M.

A4.8.1.4: A phage-encoded peptide library, a medium comprising >95% organic solvent, a CNT, and a library molecule with an affinity for a CNT of <1E−7 M.

A4.8.1.5: A phage-encoded peptide library, a medium comprising >95% organic solvent, a CNT, and a library molecule with an affinity for a CNT of <1E−8 M.

A4.8.1.6: A phage-encoded peptide library, a medium comprising >95% organic solvent, a CNT, and a library molecule with an affinity for a CNT of <1E−9 M.

A4.8.1.7: A phage-encoded peptide library, a medium comprising >95% organic solvent, a CNT, and a library molecule with an affinity for a CNT of <1E−10 M.

A4.8.1.8: A phage-encoded peptide library, a medium comprising >95% organic solvent, a CNT, and a library molecule with an affinity for a CNT of <1E−12 M.

A4.8.2.1: A phage-encoded peptide library, a medium comprising >95% organic solvent, a GS, and a library molecule with an affinity for a GS of <1E−2 M.

A4.8.2.2: A phage-encoded peptide library, a medium comprising >95% organic solvent, a GS, and a library molecule with an affinity for a GS of <1E−4 M.

A4.8.2.3: A phage-encoded peptide library, a medium comprising >95% organic solvent, a GS, and a library molecule with an affinity for a GS of <1E−6 M.

A4.8.2.4: A phage-encoded peptide library, a medium comprising >95% organic solvent, a GS, and a library molecule with an affinity for a GS of <1E−7 M.

A4.8.2.5: A phage-encoded peptide library, a medium comprising >95% organic solvent, a GS, and a library molecule with an affinity for a GS of <1E−8 M.

A4.8.2.6: A phage-encoded peptide library, a medium comprising >95% organic solvent, a GS, and a library molecule with an affinity for a GS of <1E−9 M.

A4.8.2.7: A phage-encoded peptide library, a medium comprising >95% organic solvent, a GS, and a library molecule with an affinity for a GS of <1E−10 M.

A4.8.2.8: A phage-encoded peptide library, a medium comprising >95% organic solvent, a GS, and a library molecule with an affinity for a GS of <1E−12 M.

A4.8.3.1: A phage-encoded peptide library, a medium comprising >95% organic solvent, a SWNT, and a library molecule with an affinity for a SWNT of <1E−2 M.

A4.8.3.2: A phage-encoded peptide library, a medium comprising >95% organic solvent, a SWNT, and a library molecule with an affinity for a SWNT of <1E−4 M.

A4.8.3.3: A phage-encoded peptide library, a medium comprising >95% organic solvent, a SWNT, and a library molecule with an affinity for a SWNT of <1E−6 M.

A4.8.3.4: A phage-encoded peptide library, a medium comprising >95% organic solvent, a SWNT, and a library molecule with an affinity for a SWNT of <1E−7 M.

A4.8.3.5: A phage-encoded peptide library, a medium comprising >95% organic solvent, a SWNT, and a library molecule with an affinity for a SWNT of <1E−8 M.

A4.8.3.6: A phage-encoded peptide library, a medium comprising >95% organic solvent, a SWNT, and a library molecule with an affinity for a SWNT of <1E−9 M.

A4.8.3.7: A phage-encoded peptide library, a medium comprising >95% organic solvent, a SWNT, and a library molecule with an affinity for a SWNT of <1E−10 M.

A4.8.3.8: A phage-encoded peptide library, a medium comprising >95% organic solvent, a SWNT, and a library molecule with an affinity for a SWNT of <1E−12 M.

A4.8.4.1: A phage-encoded peptide library, a medium comprising >95% organic solvent, a MWNT, and a library molecule with an affinity for a MWNT of <1E−2 M.

A4.8.4.2: A phage-encoded peptide library, a medium comprising >95% organic solvent, a MWNT, and a library molecule with an affinity for a MWNT of <1E−4 M.

A4.8.4.3: A phage-encoded peptide library, a medium comprising >95% organic solvent, a MWNT, and a library molecule with an affinity for a MWNT of <1E−6 M.

A4.8.4.4: A phage-encoded peptide library, a medium comprising >95% organic solvent, a MWNT, and a library molecule with an affinity for a MWNT of <1E−7 M.

A4.8.4.5: A phage-encoded peptide library, a medium comprising >95% organic solvent, a MWNT, and a library molecule with an affinity for a MWNT of <1E−8 M.

A4.8.4.6: A phage-encoded peptide library, a medium comprising >95% organic solvent, a MWNT, and a library molecule with an affinity for a MWNT of <1E−9 M.

A4.8.4.7: A phage-encoded peptide library, a medium comprising >95% organic solvent, a MWNT, and a library molecule with an affinity for a MWNT of <1E−10 M.

A4.8.4.8: A phage-encoded peptide library, a medium comprising >95% organic solvent, a MWNT, and a library molecule with an affinity for a MWNT of <1E−12 M.

A4.8.5.1: A phage-encoded peptide library, a medium comprising >95% organic solvent, a nanotube, and a library molecule with an affinity for a nanotube of <1E−2 M.

A4.8.5.2: A phage-encoded peptide library, a medium comprising >95% organic solvent, a nanotube, and a library molecule with an affinity for a nanotube of <1E−4 M.

A4.8.5.3: A phage-encoded peptide library, a medium comprising >95% organic solvent, a nanotube, and a library molecule with an affinity for a nanotube of <1E−6 M.

A4.8.5.4: A phage-encoded peptide library, a medium comprising >95% organic solvent, a nanotube, and a library molecule with an affinity for a nanotube of <1E−7 M.

A4.8.5.5: A phage-encoded peptide library, a medium comprising >95% organic solvent, a nanotube, and a library molecule with an affinity for a nanotube of <1E−8 M.

A4.8.5.6: A phage-encoded peptide library, a medium comprising >95% organic solvent, a nanotube, and a library molecule with an affinity for a nanotube of <1E−9 M.

A4.8.5.7: A phage-encoded peptide library, a medium comprising >95% organic solvent, a nanotube, and a library molecule with an affinity for a nanotube of <1E−10 M.

A4.8.5.8: A phage-encoded peptide library, a medium comprising >95% organic solvent, a nanotube, and a library molecule with an affinity for a nanotube of <1E−12 M.

A4.8.6.1: A phage-encoded peptide library, a medium comprising >95% organic solvent, a fullerene, and a library molecule with an affinity for a fullerene of <1E−2 M.

A4.8.6.2: A phage-encoded peptide library, a medium comprising >95% organic solvent, a fullerene, and a library molecule with an affinity for a fullerene of <1E−4 M.

A4.8.6.3: A phage-encoded peptide library, a medium comprising >95% organic solvent, a fullerene, and a library molecule with an affinity for a fullerene of <1E−6 M.

A4.8.6.4: A phage-encoded peptide library, a medium comprising >95% organic solvent, a fullerene, and a library molecule with an affinity for a fullerene of <1E−7 M.

A4.8.6.5: A phage-encoded peptide library, a medium comprising >95% organic solvent, a fullerene, and a library molecule with an affinity for a fullerene of <1E−8 M.

A4.8.6.6: A phage-encoded peptide library, a medium comprising >95% organic solvent, a fullerene, and a library molecule with an affinity for a fullerene of <1E−9 M.

A4.8.6.7: A phage-encoded peptide library, a medium comprising >95% organic solvent, a fullerene, and a library molecule with an affinity for a fullerene of <1E−10 M.

A4.8.6.8: A phage-encoded peptide library, a medium comprising >95% organic solvent, a fullerene, and a library molecule with an affinity for a fullerene of <1E−12 M.

A4.8.7.1: A phage-encoded peptide library, a medium comprising >95% organic solvent, a BNNT, and a library molecule with an affinity for a BNNT of <1E−2 M.

A4.8.7.2: A phage-encoded peptide library, a medium comprising >95% organic solvent, a BNNT, and a library molecule with an affinity for a BNNT of <1E−4 M.

A4.8.7.3: A phage-encoded peptide library, a medium comprising >95% organic solvent, a BNNT, and a library molecule with an affinity for a BNNT of <1E−6 M.

A4.8.7.4: A phage-encoded peptide library, a medium comprising >95% organic solvent, a BNNT, and a library molecule with an affinity for a BNNT of <1E−7 M.

A4.8.7.5: A phage-encoded peptide library, a medium comprising >95% organic solvent, a BNNT, and a library molecule with an affinity for a BNNT of <1E−8 M.

A4.8.7.6: A phage-encoded peptide library, a medium comprising >95% organic solvent, a BNNT, and a library molecule with an affinity for a BNNT of <1E−9 M.

A4.8.7.7: A phage-encoded peptide library, a medium comprising >95% organic solvent, a BNNT, and a library molecule with an affinity for a BNNT of <1E−10 M.

A4.8.7.8: A phage-encoded peptide library, a medium comprising >95% organic solvent, a BNNT, and a library molecule with an affinity for a BNNT of <1E−12 M.

A4.9.1.1: A phage-encoded peptide library, a medium comprising >95% organic solvent and a soluble polymer, a CNT, and a library molecule with an affinity for a CNT of <1E−2 M.

A4.9.1.2: A phage-encoded peptide library, a medium comprising >95% organic solvent and a soluble polymer, a CNT, and a library molecule with an affinity for a CNT of <1E−4 M.

A4.9.1.3: A phage-encoded peptide library, a medium comprising >95% organic solvent and a soluble polymer, a CNT, and a library molecule with an affinity for a CNT of <1E−6 M.

A4.9.1.4: A phage-encoded peptide library, a medium comprising >95% organic solvent and a soluble polymer, a CNT, and a library molecule with an affinity for a CNT of <1E−7 M.

A4.9.1.5: A phage-encoded peptide library, a medium comprising >95% organic solvent and a soluble polymer, a CNT, and a library molecule with an affinity for a CNT of <1E−8 M.

A4.9.1.6: A phage-encoded peptide library, a medium comprising >95% organic solvent and a soluble polymer, a CNT, and a library molecule with an affinity for a CNT of <1E−9 M.

A4.9.1.7: A phage-encoded peptide library, a medium comprising >95% organic solvent and a soluble polymer, a CNT, and a library molecule with an affinity for a CNT of <1E−10 M.

A4.9.1.8: A phage-encoded peptide library, a medium comprising >95% organic solvent and a soluble polymer, a CNT, and a library molecule with an affinity for a CNT of <1E−12 M.

A4.9.2.1: A phage-encoded peptide library, a medium comprising >95% organic solvent and a soluble polymer, a GS, and a library molecule with an affinity for a GS of <1E−2 M.

A4.9.2.2: A phage-encoded peptide library, a medium comprising >95% organic solvent and a soluble polymer, a GS, and a library molecule with an affinity for a GS of <1E−4 M.

A4.9.2.3: A phage-encoded peptide library, a medium comprising >95% organic solvent and a soluble polymer, a GS, and a library molecule with an affinity for a GS of <1E−6 M.

A4.9.2.4: A phage-encoded peptide library, a medium comprising >95% organic solvent and a soluble polymer, a GS, and a library molecule with an affinity for a GS of <1E−7 M.

A4.9.2.5: A phage-encoded peptide library, a medium comprising >95% organic solvent and a soluble polymer, a GS, and a library molecule with an affinity for a GS of <1E−8 M.

A4.9.2.6: A phage-encoded peptide library, a medium comprising >95% organic solvent and a soluble polymer, a GS, and a library molecule with an affinity for a GS of <1E−9 M.

A4.9.2.7: A phage-encoded peptide library, a medium comprising >95% organic solvent and a soluble polymer, a GS, and a library molecule with an affinity for a GS of <1E−10 M.

A4.9.2.8: A phage-encoded peptide library, a medium comprising >95% organic solvent and a soluble polymer, a GS, and a library molecule with an affinity for a GS of <1E−12 M.

A4.9.3.1: A phage-encoded peptide library, a medium comprising >95% organic solvent and a soluble polymer, a SWNT, and a library molecule with an affinity for a SWNT of <1E−2 M.

A4.9.3.2: A phage-encoded peptide library, a medium comprising >95% organic solvent and a soluble polymer, a SWNT, and a library molecule with an affinity for a SWNT of <1E−4 M.

A4.9.3.3: A phage-encoded peptide library, a medium comprising >95% organic solvent and a soluble polymer, a SWNT, and a library molecule with an affinity for a SWNT of <1E−6 M.

A4.9.3.4: A phage-encoded peptide library, a medium comprising >95% organic solvent and a soluble polymer, a SWNT, and a library molecule with an affinity for a SWNT of <1E−7 M.

A4.9.3.5: A phage-encoded peptide library, a medium comprising >95% organic solvent and a soluble polymer, a SWNT, and a library molecule with an affinity for a SWNT of <1E−8 M.

A4.9.3.6: A phage-encoded peptide library, a medium comprising >95% organic solvent and a soluble polymer, a SWNT, and a library molecule with an affinity for a SWNT of <1E−9 M.

A4.9.3.7: A phage-encoded peptide library, a medium comprising >95% organic solvent and a soluble polymer, a SWNT, and a library molecule with an affinity for a SWNT of <1E−10 M.

A4.9.3.8: A phage-encoded peptide library, a medium comprising >95% organic solvent and a soluble polymer, a SWNT, and a library molecule with an affinity for a SWNT of <1E−12 M.

A4.9.4.1: A phage-encoded peptide library, a medium comprising >95% organic solvent and a soluble polymer, a MWNT, and a library molecule with an affinity for a MWNT of <1E−2 M.

A4.9.4.2: A phage-encoded peptide library, a medium comprising >95% organic solvent and a soluble polymer, a MWNT, and a library molecule with an affinity for a MWNT of <1E−4 M.

A4.9.4.3: A phage-encoded peptide library, a medium comprising >95% organic solvent and a soluble polymer, a MWNT, and a library molecule with an affinity for a MWNT of <1E−6 M.

A4.9.4.4: A phage-encoded peptide library, a medium comprising >95% organic solvent and a soluble polymer, a MWNT, and a library molecule with an affinity for a MWNT of <1E−7 M.

A4.9.4.5: A phage-encoded peptide library, a medium comprising >95% organic solvent and a soluble polymer, a MWNT, and a library molecule with an affinity for a MWNT of <1E−8 M.

A4.9.4.6: A phage-encoded peptide library, a medium comprising >95% organic solvent and a soluble polymer, a MWNT, and a library molecule with an affinity for a MWNT of <1E−9 M.

A4.9.4.7: A phage-encoded peptide library, a medium comprising >95% organic solvent and a soluble polymer, a MWNT, and a library molecule with an affinity for a MWNT of <1E−10 M.

A4.9.4.8: A phage-encoded peptide library, a medium comprising >95% organic solvent and a soluble polymer, a MWNT, and a library molecule with an affinity for a MWNT of <1E−12 M.

A4.9.5.1: A phage-encoded peptide library, a medium comprising >95% organic solvent and a soluble polymer, a nanotube, and a library molecule with an affinity for a nanotube of <1E−2 M.

A4.9.5.2: A phage-encoded peptide library, a medium comprising >95% organic solvent and a soluble polymer, a nanotube, and a library molecule with an affinity for a nanotube of <1E−4 M.

A4.9.5.3: A phage-encoded peptide library, a medium comprising >95% organic solvent and a soluble polymer, a nanotube, and a library molecule with an affinity for a nanotube of <1E−6 M.

A4.9.5.4: A phage-encoded peptide library, a medium comprising >95% organic solvent and a soluble polymer, a nanotube, and a library molecule with an affinity for a nanotube of <1E−7 M.

A4.9.5.5: A phage-encoded peptide library, a medium comprising >95% organic solvent and a soluble polymer, a nanotube, and a library molecule with an affinity for a nanotube of <1E−8 M.

A4.9.5.6: A phage-encoded peptide library, a medium comprising >95% organic solvent and a soluble polymer, a nanotube, and a library molecule with an affinity for a nanotube of <1E−9 M.

A4.9.5.7: A phage-encoded peptide library, a medium comprising >95% organic solvent and a soluble polymer, a nanotube, and a library molecule with an affinity for a nanotube of <1E−10 M.

A4.9.5.8: A phage-encoded peptide library, a medium comprising >95% organic solvent and a soluble polymer, a nanotube, and a library molecule with an affinity for a nanotube of <1E−12 M.

A4.9.6.1: A phage-encoded peptide library, a medium comprising >95% organic solvent and a soluble polymer, a fullerene, and a library molecule with an affinity for a fullerene of <1E−2 M.

A4.9.6.2: A phage-encoded peptide library, a medium comprising >95% organic solvent and a soluble polymer, a fullerene, and a library molecule with an affinity for a fullerene of <1E−4 M.

A4.9.6.3: A phage-encoded peptide library, a medium comprising >95% organic solvent and a soluble polymer, a fullerene, and a library molecule with an affinity for a fullerene of <1E−6 M.

A4.9.6.4: A phage-encoded peptide library, a medium comprising >95% organic solvent and a soluble polymer, a fullerene, and a library molecule with an affinity for a fullerene of <1E−7 M.

A4.9.6.5: A phage-encoded peptide library, a medium comprising >95% organic solvent and a soluble polymer, a fullerene, and a library molecule with an affinity for a fullerene of <1E−8 M.

A4.9.6.6: A phage-encoded peptide library, a medium comprising >95% organic solvent and a soluble polymer, a fullerene, and a library molecule with an affinity for a fullerene of <1E−9 M.

A4.9.6.7: A phage-encoded peptide library, a medium comprising >95% organic solvent and a soluble polymer, a fullerene, and a library molecule with an affinity for a fullerene of <1E−10 M.

A4.9.6.8: A phage-encoded peptide library, a medium comprising >95% organic solvent and a soluble polymer, a fullerene, and a library molecule with an affinity for a fullerene of <1E−12 M.

A4.9.7.1: A phage-encoded peptide library, a medium comprising >95% organic solvent and a soluble polymer, a BNNT, and a library molecule with an affinity for a BNNT of <1E−2 M.

A4.9.7.2: A phage-encoded peptide library, a medium comprising >95% organic solvent and a soluble polymer, a BNNT, and a library molecule with an affinity for a BNNT of <1E−4 M.

A4.9.7.3: A phage-encoded peptide library, a medium comprising >95% organic solvent and a soluble polymer, a BNNT, and a library molecule with an affinity for a BNNT of <1E−6 M.

A4.9.7.4: A phage-encoded peptide library, a medium comprising >95% organic solvent and a soluble polymer, a BNNT, and a library molecule with an affinity for a BNNT of <1E−7 M.

A4.9.7.5: A phage-encoded peptide library, a medium comprising >95% organic solvent and a soluble polymer, a BNNT, and a library molecule with an affinity for a BNNT of <1E−8 M.

A4.9.7.6: A phage-encoded peptide library, a medium comprising >95% organic solvent and a soluble polymer, a BNNT, and a library molecule with an affinity for a BNNT of <1E−9 M.

A4.9.7.7: A phage-encoded peptide library, a medium comprising >95% organic solvent and a soluble polymer, a BNNT, and a library molecule with an affinity for a BNNT of <1E−10 M.

A4.9.7.8: A phage-encoded peptide library, a medium comprising >95% organic solvent and a soluble polymer, a BNNT, and a library molecule with an affinity for a BNNT of <1E−12 M.

A5.1.1.1: A polysome-encoded peptide library, a medium comprising an organic solvent, a CNT, and a library molecule with an affinity for a CNT of <1E−2 M.

A5.1.1.2: A polysome-encoded peptide library, a medium comprising an organic solvent, a CNT, and a library molecule with an affinity for a CNT of <1E−4 M.

A5.1.1.3: A polysome-encoded peptide library, a medium comprising an organic solvent, a CNT, and a library molecule with an affinity for a CNT of <1E−6 M.

A5.1.1.4: A polysome-encoded peptide library, a medium comprising an organic solvent, a CNT, and a library molecule with an affinity for a CNT of <1E−7 M.

A5.1.1.5: A polysome-encoded peptide library, a medium comprising an organic solvent, a CNT, and a library molecule with an affinity for a CNT of <1E−8 M.

A5.1.1.6: A polysome-encoded peptide library, a medium comprising an organic solvent, a CNT, and a library molecule with an affinity for a CNT of <1E−9 M.

A5.1.1.7: A polysome-encoded peptide library, a medium comprising an organic solvent, a CNT, and a library molecule with an affinity for a CNT of <1E−10 M.

A5.1.1.8: A polysome-encoded peptide library, a medium comprising an organic solvent, a CNT, and a library molecule with an affinity for a CNT of <1E−12 M.

A5.1.2.1: A polysome-encoded peptide library, a medium comprising an organic solvent, a GS, and a library molecule with an affinity for a GS of <1E−2 M.

A5.1.2.2: A polysome-encoded peptide library, a medium comprising an organic solvent, a GS, and a library molecule with an affinity for a GS of <1E−4 M.

A5.1.2.3: A polysome-encoded peptide library, a medium comprising an organic solvent, a GS, and a library molecule with an affinity for a GS of <1E−6 M.

A5.1.2.4: A polysome-encoded peptide library, a medium comprising an organic solvent, a GS, and a library molecule with an affinity for a GS of <1E−7 M.

A5.1.2.5: A polysome-encoded peptide library, a medium comprising an organic solvent, a GS, and a library molecule with an affinity for a GS of <1E−8 M.

A5.1.2.6: A polysome-encoded peptide library, a medium comprising an organic solvent, a GS, and a library molecule with an affinity for a GS of <1E−9 M.

A5.1.2.7: A polysome-encoded peptide library, a medium comprising an organic solvent, a GS, and a library molecule with an affinity for a GS of <1E−10 M.

A5.1.2.8: A polysome-encoded peptide library, a medium comprising an organic solvent, a GS, and a library molecule with an affinity for a GS of <1E−12 M.

A5.1.3.1: A polysome-encoded peptide library, a medium comprising an organic solvent, a SWNT, and a library molecule with an affinity for a SWNT of <1E−2 M.

A5.1.3.2: A polysome-encoded peptide library, a medium comprising an organic solvent, a SWNT, and a library molecule with an affinity for a SWNT of <1E−4 M.

A5.1.3.3: A polysome-encoded peptide library, a medium comprising an organic solvent, a SWNT, and a library molecule with an affinity for a SWNT of <1E−6 M.

A5.1.3.4: A polysome-encoded peptide library, a medium comprising an organic solvent, a SWNT, and a library molecule with an affinity for a SWNT of <1E−7 M.

A5.1.3.5: A polysome-encoded peptide library, a medium comprising an organic solvent, a SWNT, and a library molecule with an affinity for a SWNT of <1E−8 M.

A5.1.3.6: A polysome-encoded peptide library, a medium comprising an organic solvent, a SWNT, and a library molecule with an affinity for a SWNT of <1E−9 M.

A5.1.3.7: A polysome-encoded peptide library, a medium comprising an organic solvent, a SWNT, and a library molecule with an affinity for a SWNT of <1E−10 M.

A5.1.3.8: A polysome-encoded peptide library, a medium comprising an organic solvent, a SWNT, and a library molecule with an affinity for a SWNT of <1E−12 M.

A5.1.4.1: A polysome-encoded peptide library, a medium comprising an organic solvent, a MWNT, and a library molecule with an affinity for a MWNT of <1E−2 M.

A5.1.4.2: A polysome-encoded peptide library, a medium comprising an organic solvent, a MWNT, and a library molecule with an affinity for a MWNT of <1E−4 M.

A5.1.4.3: A polysome-encoded peptide library, a medium comprising an organic solvent, a MWNT, and a library molecule with an affinity for a MWNT of <1E−6 M.

A5.1.4.4: A polysome-encoded peptide library, a medium comprising an organic solvent, a MWNT, and a library molecule with an affinity for a MWNT of <1E−7 M.

A5.1.4.5: A polysome-encoded peptide library, a medium comprising an organic solvent, a MWNT, and a library molecule with an affinity for a MWNT of <1E−8 M.

A5.1.4.6: A polysome-encoded peptide library, a medium comprising an organic solvent, a MWNT, and a library molecule with an affinity for a MWNT of <1E−9 M.

A5.1.4.7: A polysome-encoded peptide library, a medium comprising an organic solvent, a MWNT, and a library molecule with an affinity for a MWNT of <1E−10 M.

A5.1.4.8: A polysome-encoded peptide library, a medium comprising an organic solvent, a MWNT, and a library molecule with an affinity for a MWNT of <1E−12 M.

A5.1.5.1: A polysome-encoded peptide library, a medium comprising an organic solvent, a nanotube, and a library molecule with an affinity for a nanotube of <1E−2 M.

A5.1.5.2: A polysome-encoded peptide library, a medium comprising an organic solvent, a nanotube, and a library molecule with an affinity for a nanotube of <1E−4 M.

A5.1.5.3: A polysome-encoded peptide library, a medium comprising an organic solvent, a nanotube, and a library molecule with an affinity for a nanotube of <1E−6 M.

A5.1.5.4: A polysome-encoded peptide library, a medium comprising an organic solvent, a nanotube, and a library molecule with an affinity for a nanotube of <1E−7 M.

A5.1.5.5: A polysome-encoded peptide library, a medium comprising an organic solvent, a nanotube, and a library molecule with an affinity for a nanotube of <1E−8 M.

A5.1.5.6: A polysome-encoded peptide library, a medium comprising an organic solvent, a nanotube, and a library molecule with an affinity for a nanotube of <1E−9 M.

A5.1.5.7: A polysome-encoded peptide library, a medium comprising an organic solvent, a nanotube, and a library molecule with an affinity for a nanotube of <1E−10 M.

A5.1.5.8: A polysome-encoded peptide library, a medium comprising an organic solvent, a nanotube, and a library molecule with an affinity for a nanotube of <1E−12 M.

A5.1.6.1: A polysome-encoded peptide library, a medium comprising an organic solvent, a fullerene, and a library molecule with an affinity for a fullerene of <1E−2 M.

A5.1.6.2: A polysome-encoded peptide library, a medium comprising an organic solvent, a fullerene, and a library molecule with an affinity for a fullerene of <1E−4 M.

A5.1.6.3: A polysome-encoded peptide library, a medium comprising an organic solvent, a fullerene, and a library molecule with an affinity for a fullerene of <1E−6 M.

A5.1.6.4: A polysome-encoded peptide library, a medium comprising an organic solvent, a fullerene, and a library molecule with an affinity for a fullerene of <1E−7 M.

A5.1.6.5: A polysome-encoded peptide library, a medium comprising an organic solvent, a fullerene, and a library molecule with an affinity for a fullerene of <1E−8 M.

A5.1.6.6: A polysome-encoded peptide library, a medium comprising an organic solvent, a fullerene, and a library molecule with an affinity for a fullerene of <1E−9 M.

A5.1.6.7: A polysome-encoded peptide library, a medium comprising an organic solvent, a fullerene, and a library molecule with an affinity for a fullerene of <1E−10 M.

A5.1.6.8: A polysome-encoded peptide library, a medium comprising an organic solvent, a fullerene, and a library molecule with an affinity for a fullerene of <1E−12 M.

A5.1.7.1: A polysome-encoded peptide library, a medium comprising an organic solvent, a BNNT, and a library molecule with an affinity for a BNNT of <1E−2 M.

A5.1.7.2: A polysome-encoded peptide library, a medium comprising an organic solvent, a BNNT, and a library molecule with an affinity for a BNNT of <1E−4 M.

A5.1.7.3: A polysome-encoded peptide library, a medium comprising an organic solvent, a BNNT, and a library molecule with an affinity for a BNNT of <1E−6 M.

A5.1.7.4: A polysome-encoded peptide library, a medium comprising an organic solvent, a BNNT, and a library molecule with an affinity for a BNNT of <1E−7 M.

A5.1.7.5: A polysome-encoded peptide library, a medium comprising an organic solvent, a BNNT, and a library molecule with an affinity for a BNNT of <1E−8 M.

A5.1.7.6: A polysome-encoded peptide library, a medium comprising an organic solvent, a BNNT, and a library molecule with an affinity for a BNNT of <1E−9 M.

A5.1.7.7: A polysome-encoded peptide library, a medium comprising an organic solvent, a BNNT, and a library molecule with an affinity for a BNNT of <1E−10 M.

A5.1.7.8: A polysome-encoded peptide library, a medium comprising an organic solvent, a BNNT, and a library molecule with an affinity for a BNNT of <1E−12 M.

A5.2.1.1: A polysome-encoded peptide library, a medium comprising an organic solvent and a soluble polymer, a CNT, and a library molecule with an affinity for a CNT of <1E−2 M.

A5.2.1.2: A polysome-encoded peptide library, a medium comprising an organic solvent and a soluble polymer, a CNT, and a library molecule with an affinity for a CNT of <1E−4 M.

A5.2.1.3: A polysome-encoded peptide library, a medium comprising an organic solvent and a soluble polymer, a CNT, and a library molecule with an affinity for a CNT of <1E−6 M.

A5.2.1.4: A polysome-encoded peptide library, a medium comprising an organic solvent and a soluble polymer, a CNT, and a library molecule with an affinity for a CNT of <1E−7 M.

A5.2.1.5: A polysome-encoded peptide library, a medium comprising an organic solvent and a soluble polymer, a CNT, and a library molecule with an affinity for a CNT of <1E−8 M.

A5.2.1.6: A polysome-encoded peptide library, a medium comprising an organic solvent and a soluble polymer, a CNT, and a library molecule with an affinity for a CNT of <1E−9 M.

A5.2.1.7: A polysome-encoded peptide library, a medium comprising an organic solvent and a soluble polymer, a CNT, and a library molecule with an affinity for a CNT of <1E−10 M.

A5.2.1.8: A polysome-encoded peptide library, a medium comprising an organic solvent and a soluble polymer, a CNT, and a library molecule with an affinity for a CNT of <1E−12 M.

A5.2.2.1: A polysome-encoded peptide library, a medium comprising an organic solvent and a soluble polymer, a GS, and a library molecule with an affinity for a GS of <1E−2 M.

A5.2.2.2: A polysome-encoded peptide library, a medium comprising an organic solvent and a soluble polymer, a GS, and a library molecule with an affinity for a GS of <1E−4 M.

A5.2.2.3: A polysome-encoded peptide library, a medium comprising an organic solvent and a soluble polymer, a GS, and a library molecule with an affinity for a GS of <1E−6 M.

A5.2.2.4: A polysome-encoded peptide library, a medium comprising an organic solvent and a soluble polymer, a GS, and a library molecule with an affinity for a GS of <1E−7 M.

A5.2.2.5: A polysome-encoded peptide library, a medium comprising an organic solvent and a soluble polymer, a GS, and a library molecule with an affinity for a GS of <1E−8 M.

A5.2.2.6: A polysome-encoded peptide library, a medium comprising an organic solvent and a soluble polymer, a GS, and a library molecule with an affinity for a GS of <1E−9 M.

A5.2.2.7: A polysome-encoded peptide library, a medium comprising an organic solvent and a soluble polymer, a GS, and a library molecule with an affinity for a GS of <1E−10 M.

A5.2.2.8: A polysome-encoded peptide library, a medium comprising an organic solvent and a soluble polymer, a GS, and a library molecule with an affinity for a GS of <1E−12 M.

A5.2.3.1: A polysome-encoded peptide library, a medium comprising an organic solvent and a soluble polymer, a SWNT, and a library molecule with an affinity for a SWNT of <1E−2 M.

A5.2.3.2: A polysome-encoded peptide library, a medium comprising an organic solvent and a soluble polymer, a SWNT, and a library molecule with an affinity for a SWNT of <1E−4 M.

A5.2.3.3: A polysome-encoded peptide library, a medium comprising an organic solvent and a soluble polymer, a SWNT, and a library molecule with an affinity for a SWNT of <1E−6 M.

A5.2.3.4: A polysome-encoded peptide library, a medium comprising an organic solvent and a soluble polymer, a SWNT, and a library molecule with an affinity for a SWNT of <1E−7 M.

A5.2.3.5: A polysome-encoded peptide library, a medium comprising an organic solvent and a soluble polymer, a SWNT, and a library molecule with an affinity for a SWNT of <1E−8 M.

A5.2.3.6: A polysome-encoded peptide library, a medium comprising an organic solvent and a soluble polymer, a SWNT, and a library molecule with an affinity for a SWNT of <1E−9 M.

A5.2.3.7: A polysome-encoded peptide library, a medium comprising an organic solvent and a soluble polymer, a SWNT, and a library molecule with an affinity for a SWNT of <1E−10 M.

A5.2.3.8: A polysome-encoded peptide library, a medium comprising an organic solvent and a soluble polymer, a SWNT, and a library molecule with an affinity for a SWNT of <1E−12 M.

A5.2.4.1: A polysome-encoded peptide library, a medium comprising an organic solvent and a soluble polymer, a MWNT, and a library molecule with an affinity for a MWNT of <1E−2 M.

A5.2.4.2: A polysome-encoded peptide library, a medium comprising an organic solvent and a soluble polymer, a MWNT, and a library molecule with an affinity for a MWNT of <1E−4 M.

A5.2.4.3: A polysome-encoded peptide library, a medium comprising an organic solvent and a soluble polymer, a MWNT, and a library molecule with an affinity for a MWNT of <1E−6 M.

A5.2.4.4: A polysome-encoded peptide library, a medium comprising an organic solvent and a soluble polymer, a MWNT, and a library molecule with an affinity for a MWNT of <1E−7 M.

A5.2.4.5: A polysome-encoded peptide library, a medium comprising an organic solvent and a soluble polymer, a MWNT, and a library molecule with an affinity for a MWNT of <1E−8 M.

A5.2.4.6: A polysome-encoded peptide library, a medium comprising an organic solvent and a soluble polymer, a MWNT, and a library molecule with an affinity for a MWNT of <1E−9 M.

A5.2.4.7: A polysome-encoded peptide library, a medium comprising an organic solvent and a soluble polymer, a MWNT, and a library molecule with an affinity for a MWNT of <1E−10 M.

A5.2.4.8: A polysome-encoded peptide library, a medium comprising an organic solvent and a soluble polymer, a MWNT, and a library molecule with an affinity for a MWNT of <1E−12 M.

A5.2.5.1: A polysome-encoded peptide library, a medium comprising an organic solvent and a soluble polymer, a nanotube, and a library molecule with an affinity for a nanotube of <1E−2 M.

A5.2.5.2: A polysome-encoded peptide library, a medium comprising an organic solvent and a soluble polymer, a nanotube, and a library molecule with an affinity for a nanotube of <1E−4 M.

A5.2.5.3: A polysome-encoded peptide library, a medium comprising an organic solvent and a soluble polymer, a nanotube, and a library molecule with an affinity for a nanotube of <1E−6 M.

A5.2.5.4: A polysome-encoded peptide library, a medium comprising an organic solvent and a soluble polymer, a nanotube, and a library molecule with an affinity for a nanotube of <1E−7 M.

A5.2.5.5: A polysome-encoded peptide library, a medium comprising an organic solvent and a soluble polymer, a nanotube, and a library molecule with an affinity for a nanotube of <1E−8 M.

A5.2.5.6: A polysome-encoded peptide library, a medium comprising an organic solvent and a soluble polymer, a nanotube, and a library molecule with an affinity for a nanotube of <1E−9 M.

A5.2.5.7: A polysome-encoded peptide library, a medium comprising an organic solvent and a soluble polymer, a nanotube, and a library molecule with an affinity for a nanotube of <1E−10 M.

A5.2.5.8: A polysome-encoded peptide library, a medium comprising an organic solvent and a soluble polymer, a nanotube, and a library molecule with an affinity for a nanotube of <1E−12 M.

A5.2.6.1: A polysome-encoded peptide library, a medium comprising an organic solvent and a soluble polymer, a fullerene, and a library molecule with an affinity for a fullerene of <1E−2 M.

A5.2.6.2: A polysome-encoded peptide library, a medium comprising an organic solvent and a soluble polymer, a fullerene, and a library molecule with an affinity for a fullerene of <1E−4 M.

A5.2.6.3: A polysome-encoded peptide library, a medium comprising an organic solvent and a soluble polymer, a fullerene, and a library molecule with an affinity for a fullerene of <1E−6 M.

A5.2.6.4: A polysome-encoded peptide library, a medium comprising an organic solvent and a soluble polymer, a fullerene, and a library molecule with an affinity for a fullerene of <1E−7 M.

A5.2.6.5: A polysome-encoded peptide library, a medium comprising an organic solvent and a soluble polymer, a fullerene, and a library molecule with an affinity for a fullerene of <1E−8 M.

A5.2.6.6: A polysome-encoded peptide library, a medium comprising an organic solvent and a soluble polymer, a fullerene, and a library molecule with an affinity for a fullerene of <1E−9 M.

A5.2.6.7: A polysome-encoded peptide library, a medium comprising an organic solvent and a soluble polymer, a fullerene, and a library molecule with an affinity for a fullerene of <1E−10 M.

A5.2.6.8: A polysome-encoded peptide library, a medium comprising an organic solvent and a soluble polymer, a fullerene, and a library molecule with an affinity for a fullerene of <1E−12 M.

A5.2.7.1: A polysome-encoded peptide library, a medium comprising an organic solvent and a soluble polymer, a BNNT, and a library molecule with an affinity for a BNNT of <1E−2 M.

A5.2.7.2: A polysome-encoded peptide library, a medium comprising an organic solvent and a soluble polymer, a BNNT, and a library molecule with an affinity for a BNNT of <1E−4 M.

A5.2.7.3: A polysome-encoded peptide library, a medium comprising an organic solvent and a soluble polymer, a BNNT, and a library molecule with an affinity for a BNNT of <1E−6 M.

A5.2.7.4: A polysome-encoded peptide library, a medium comprising an organic solvent and a soluble polymer, a BNNT, and a library molecule with an affinity for a BNNT of <1E−7 M.

A5.2.7.5: A polysome-encoded peptide library, a medium comprising an organic solvent and a soluble polymer, a BNNT, and a library molecule with an affinity for a BNNT of <1E−8 M.

A5.2.7.6: A polysome-encoded peptide library, a medium comprising an organic solvent and a soluble polymer, a BNNT, and a library molecule with an affinity for a BNNT of <1E−9 M.

A5.2.7.7: A polysome-encoded peptide library, a medium comprising an organic solvent and a soluble polymer, a BNNT, and a library molecule with an affinity for a BNNT of <1E−10 M.

A5.2.7.8: A polysome-encoded peptide library, a medium comprising an organic solvent and a soluble polymer, a BNNT, and a library molecule with an affinity for a BNNT of <1E−12 M.

A5.3.1.1: A polysome-encoded peptide library, a medium comprising a soluble polymer, a CNT, and a library molecule with an affinity for a CNT of <1E−2 M.

A5.3.1.2: A polysome-encoded peptide library, a medium comprising a soluble polymer, a CNT, and a library molecule with an affinity for a CNT of <1E−4 M.

A5.3.1.3: A polysome-encoded peptide library, a medium comprising a soluble polymer, a CNT, and a library molecule with an affinity for a CNT of <1E−6 M.

A5.3.1.4: A polysome-encoded peptide library, a medium comprising a soluble polymer, a CNT, and a library molecule with an affinity for a CNT of <1E−7 M.

A5.3.1.5: A polysome-encoded peptide library, a medium comprising a soluble polymer, a CNT, and a library molecule with an affinity for a CNT of <1E−8 M.

A5.3.1.6: A polysome-encoded peptide library, a medium comprising a soluble polymer, a CNT, and a library molecule with an affinity for a CNT of <1E−9 M.

A5.3.1.7: A polysome-encoded peptide library, a medium comprising a soluble polymer, a CNT, and a library molecule with an affinity for a CNT of <1E−10 M.

A5.3.1.8: A polysome-encoded peptide library, a medium comprising a soluble polymer, a CNT, and a library molecule with an affinity for a CNT of <1E−12 M.

A5.3.2.1: A polysome-encoded peptide library, a medium comprising a soluble polymer, a GS, and a library molecule with an affinity for a GS of <1E−2 M.

A5.3.2.2: A polysome-encoded peptide library, a medium comprising a soluble polymer, a GS, and a library molecule with an affinity for a GS of <1E−4 M.

A5.3.2.3: A polysome-encoded peptide library, a medium comprising a soluble polymer, a GS, and a library molecule with an affinity for a GS of <1E−6 M.

A5.3.2.4: A polysome-encoded peptide library, a medium comprising a soluble polymer, a GS, and a library molecule with an affinity for a GS of <1E−7 M.

A5.3.2.5: A polysome-encoded peptide library, a medium comprising a soluble polymer, a GS, and a library molecule with an affinity for a GS of <1E−8 M.

A5.3.2.6: A polysome-encoded peptide library, a medium comprising a soluble polymer, a GS, and a library molecule with an affinity for a GS of <1E−9 M.

A5.3.2.7: A polysome-encoded peptide library, a medium comprising a soluble polymer, a GS, and a library molecule with an affinity for a GS of <1E−10 M.

A5.3.2.8: A polysome-encoded peptide library, a medium comprising a soluble polymer, a GS, and a library molecule with an affinity for a GS of <1E−12 M.

A5.3.3.1: A polysome-encoded peptide library, a medium comprising a soluble polymer, a SWNT, and a library molecule with an affinity for a SWNT of <1E−2 M.

A5.3.3.2: A polysome-encoded peptide library, a medium comprising a soluble polymer, a SWNT, and a library molecule with an affinity for a SWNT of <1E−4 M.

A5.3.3.3: A polysome-encoded peptide library, a medium comprising a soluble polymer, a SWNT, and a library molecule with an affinity for a SWNT of <1E−6 M.

A5.3.3.4: A polysome-encoded peptide library, a medium comprising a soluble polymer, a SWNT, and a library molecule with an affinity for a SWNT of <1E−7 M.

A5.3.3.5: A polysome-encoded peptide library, a medium comprising a soluble polymer, a SWNT, and a library molecule with an affinity for a SWNT of <1E−8 M.

A5.3.3.6: A polysome-encoded peptide library, a medium comprising a soluble polymer, a SWNT, and a library molecule with an affinity for a SWNT of <1E−9 M.

A5.3.3.7: A polysome-encoded peptide library, a medium comprising a soluble polymer, a SWNT, and a library molecule with an affinity for a SWNT of <1E−10 M.

A5.3.3.8: A polysome-encoded peptide library, a medium comprising a soluble polymer, a SWNT, and a library molecule with an affinity for a SWNT of <1E−12 M.

A5.3.4.1: A polysome-encoded peptide library, a medium comprising a soluble polymer, a MWNT, and a library molecule with an affinity for a MWNT of <1E−2 M.

A5.3.4.2: A polysome-encoded peptide library, a medium comprising a soluble polymer, a MWNT, and a library molecule with an affinity for a MWNT of <1E−4 M.

A5.3.4.3: A polysome-encoded peptide library, a medium comprising a soluble polymer, a MWNT, and a library molecule with an affinity for a MWNT of <1E−6 M.

A5.3.4.4: A polysome-encoded peptide library, a medium comprising a soluble polymer, a MWNT, and a library molecule with an affinity for a MWNT of <1E−7 M.

A5.3.4.5: A polysome-encoded peptide library, a medium comprising a soluble polymer, a MWNT, and a library molecule with an affinity for a MWNT of <1E−8 M.

A5.3.4.6: A polysome-encoded peptide library, a medium comprising a soluble polymer, a MWNT, and a library molecule with an affinity for a MWNT of <1E−9 M.

A5.3.4.7: A polysome-encoded peptide library, a medium comprising a soluble polymer, a MWNT, and a library molecule with an affinity for a MWNT of <1E−10 M.

A5.3.4.8: A polysome-encoded peptide library, a medium comprising a soluble polymer, a MWNT, and a library molecule with an affinity for a MWNT of <1E−12 M.

A5.3.5.1: A polysome-encoded peptide library, a medium comprising a soluble polymer, a nanotube, and a library molecule with an affinity for a nanotube of <1E−2 M.

A5.3.5.2: A polysome-encoded peptide library, a medium comprising a soluble polymer, a nanotube, and a library molecule with an affinity for a nanotube of <1E−4 M.

A5.3.5.3: A polysome-encoded peptide library, a medium comprising a soluble polymer, a nanotube, and a library molecule with an affinity for a nanotube of <1E−6 M.

A5.3.5.4: A polysome-encoded peptide library, a medium comprising a soluble polymer, a nanotube, and a library molecule with an affinity for a nanotube of <1E−7 M.

A5.3.5.5: A polysome-encoded peptide library, a medium comprising a soluble polymer, a nanotube, and a library molecule with an affinity for a nanotube of <1E−8 M.

A5.3.5.6: A polysome-encoded peptide library, a medium comprising a soluble polymer, a nanotube, and a library molecule with an affinity for a nanotube of <1E−9 M.

A5.3.5.7: A polysome-encoded peptide library, a medium comprising a soluble polymer, a nanotube, and a library molecule with an affinity for a nanotube of <1E−10 M.

A5.3.5.8: A polysome-encoded peptide library, a medium comprising a soluble polymer, a nanotube, and a library molecule with an affinity for a nanotube of <1E−12 M.

A5.3.6.1: A polysome-encoded peptide library, a medium comprising a soluble polymer, a fullerene, and a library molecule with an affinity for a fullerene of <1E−2 M.

A5.3.6.2: A polysome-encoded peptide library, a medium comprising a soluble polymer, a fullerene, and a library molecule with an affinity for a fullerene of <1E−4 M.

A5.3.6.3: A polysome-encoded peptide library, a medium comprising a soluble polymer, a fullerene, and a library molecule with an affinity for a fullerene of <1E−6 M.

A5.3.6.4: A polysome-encoded peptide library, a medium comprising a soluble polymer, a fullerene, and a library molecule with an affinity for a fullerene of <1E−7 M.

A5.3.6.5: A polysome-encoded peptide library, a medium comprising a soluble polymer, a fullerene, and a library molecule with an affinity for a fullerene of <1E−8 M.

A5.3.6.6: A polysome-encoded peptide library, a medium comprising a soluble polymer, a fullerene, and a library molecule with an affinity for a fullerene of <1E−9 M.

A5.3.6.7: A polysome-encoded peptide library, a medium comprising a soluble polymer, a fullerene, and a library molecule with an affinity for a fullerene of <1E−10 M.

A5.3.6.8: A polysome-encoded peptide library, a medium comprising a soluble polymer, a fullerene, and a library molecule with an affinity for a fullerene of <1E−12 M.

A5.3.7.1: A polysome-encoded peptide library, a medium comprising a soluble polymer, a BNNT, and a library molecule with an affinity for a BNNT of <1E−2 M.

A5.3.7.2: A polysome-encoded peptide library, a medium comprising a soluble polymer, a BNNT, and a library molecule with an affinity for a BNNT of <1E−4 M.

A5.3.7.3: A polysome-encoded peptide library, a medium comprising a soluble polymer, a BNNT, and a library molecule with an affinity for a BNNT of <1E−6 M.

A5.3.7.4: A polysome-encoded peptide library, a medium comprising a soluble polymer, a BNNT, and a library molecule with an affinity for a BNNT of <1E−7 M.

A5.3.7.5: A polysome-encoded peptide library, a medium comprising a soluble polymer, a BNNT, and a library molecule with an affinity for a BNNT of <1E−8 M.

A5.3.7.6: A polysome-encoded peptide library, a medium comprising a soluble polymer, a BNNT, and a library molecule with an affinity for a BNNT of <1E−9 M.

A5.3.7.7: A polysome-encoded peptide library, a medium comprising a soluble polymer, a BNNT, and a library molecule with an affinity for a BNNT of <1E−10 M.

A5.3.7.8: A polysome-encoded peptide library, a medium comprising a soluble polymer, a BNNT, and a library molecule with an affinity for a BNNT of <1E−12 M.

A5.4.1.1: A polysome-encoded peptide library, a medium comprising >50% organic solvent, a CNT, and a library molecule with an affinity for a CNT of <1E−2 M.

A5.4.1.2: A polysome-encoded peptide library, a medium comprising >50% organic solvent, a CNT, and a library molecule with an affinity for a CNT of <1E−4 M.

A5.4.1.3: A polysome-encoded peptide library, a medium comprising >50% organic solvent, a CNT, and a library molecule with an affinity for a CNT of <1E−6 M.

A5.4.1.4: A polysome-encoded peptide library, a medium comprising >50% organic solvent, a CNT, and a library molecule with an affinity for a CNT of <1E−7 M.

A5.4.1.5: A polysome-encoded peptide library, a medium comprising >50% organic solvent, a CNT, and a library molecule with an affinity for a CNT of <1E−8 M.

A5.4.1.6: A polysome-encoded peptide library, a medium comprising >50% organic solvent, a CNT, and a library molecule with an affinity for a CNT of <1E−9 M.

A5.4.1.7: A polysome-encoded peptide library, a medium comprising >50% organic solvent, a CNT, and a library molecule with an affinity for a CNT of <1E−10 M.

A5.4.1.8: A polysome-encoded peptide library, a medium comprising >50% organic solvent, a CNT, and a library molecule with an affinity for a CNT of <1E−12 M.

A5.4.2.1: A polysome-encoded peptide library, a medium comprising >50% organic solvent, a GS, and a library molecule with an affinity for a GS of <1E−2 M.

A5.4.2.2: A polysome-encoded peptide library, a medium comprising >50% organic solvent, a GS, and a library molecule with an affinity for a GS of <1E−4 M.

A5.4.2.3: A polysome-encoded peptide library, a medium comprising >50% organic solvent, a GS, and a library molecule with an affinity for a GS of <1E−6 M.

A5.4.2.4: A polysome-encoded peptide library, a medium comprising >50% organic solvent, a GS, and a library molecule with an affinity for a GS of <1E−7 M.

A5.4.2.5: A polysome-encoded peptide library, a medium comprising >50% organic solvent, a GS, and a library molecule with an affinity for a GS of <1E−8 M.

A5.4.2.6: A polysome-encoded peptide library, a medium comprising >50% organic solvent, a GS, and a library molecule with an affinity for a GS of <1E−9 M.

A5.4.2.7: A polysome-encoded peptide library, a medium comprising >50% organic solvent, a GS, and a library molecule with an affinity for a GS of <1E−10 M.

A5.4.2.8: A polysome-encoded peptide library, a medium comprising >50% organic solvent, a GS, and a library molecule with an affinity for a GS of <1E−12 M.

A5.4.3.1: A polysome-encoded peptide library, a medium comprising >50% organic solvent, a SWNT, and a library molecule with an affinity for a SWNT of <1E−2 M.

A5.4.3.2: A polysome-encoded peptide library, a medium comprising >50% organic solvent, a SWNT, and a library molecule with an affinity for a SWNT of <1E−4 M.

A5.4.3.3: A polysome-encoded peptide library, a medium comprising >50% organic solvent, a SWNT, and a library molecule with an affinity for a SWNT of <1E−6 M.

A5.4.3.4: A polysome-encoded peptide library, a medium comprising >50% organic solvent, a SWNT, and a library molecule with an affinity for a SWNT of <1E−7 M.

A5.4.3.5: A polysome-encoded peptide library, a medium comprising >50% organic solvent, a SWNT, and a library molecule with an affinity for a SWNT of <1E−8 M.

A5.4.3.6: A polysome-encoded peptide library, a medium comprising >50% organic solvent, a SWNT, and a library molecule with an affinity for a SWNT of <1E−9 M.

A5.4.3.7: A polysome-encoded peptide library, a medium comprising >50% organic solvent, a SWNT, and a library molecule with an affinity for a SWNT of <1E−10 M.

A5.4.3.8: A polysome-encoded peptide library, a medium comprising >50% organic solvent, a SWNT, and a library molecule with an affinity for a SWNT of <1E−12 M.

A5.4.4.1: A polysome-encoded peptide library, a medium comprising >50% organic solvent, a MWNT, and a library molecule with an affinity for a MWNT of <1E−2 M.

A5.4.4.2: A polysome-encoded peptide library, a medium comprising >50% organic solvent, a MWNT, and a library molecule with an affinity for a MWNT of <1E−4 M.

A5.4.4.3: A polysome-encoded peptide library, a medium comprising >50% organic solvent, a MWNT, and a library molecule with an affinity for a MWNT of <1E−6 M.

A5.4.4.4: A polysome-encoded peptide library, a medium comprising >50% organic solvent, a MWNT, and a library molecule with an affinity for a MWNT of <1E−7 M.

A5.4.4.5: A polysome-encoded peptide library, a medium comprising >50% organic solvent, a MWNT, and a library molecule with an affinity for a MWNT of <1E−8 M.

A5.4.4.6: A polysome-encoded peptide library, a medium comprising >50% organic solvent, a MWNT, and a library molecule with an affinity for a MWNT of <1E−9 M.

A5.4.4.7: A polysome-encoded peptide library, a medium comprising >50% organic solvent, a MWNT, and a library molecule with an affinity for a MWNT of <1E−10 M.

A5.4.4.8: A polysome-encoded peptide library, a medium comprising >50% organic solvent, a MWNT, and a library molecule with an affinity for a MWNT of <1E−12 M.

A5.4.5.1: A polysome-encoded peptide library, a medium comprising >50% organic solvent, a nanotube, and a library molecule with an affinity for a nanotube of <1E−2 M.

A5.4.5.2: A polysome-encoded peptide library, a medium comprising >50% organic solvent, a nanotube, and a library molecule with an affinity for a nanotube of <1E−4 M.

A5.4.5.3: A polysome-encoded peptide library, a medium comprising >50% organic solvent, a nanotube, and a library molecule with an affinity for a nanotube of <1E−6 M.

A5.4.5.4: A polysome-encoded peptide library, a medium comprising >50% organic solvent, a nanotube, and a library molecule with an affinity for a nanotube of <1E−7 M.

A5.4.5.5: A polysome-encoded peptide library, a medium comprising >50% organic solvent, a nanotube, and a library molecule with an affinity for a nanotube of <1E−8 M.

A5.4.5.6: A polysome-encoded peptide library, a medium comprising >50% organic solvent, a nanotube, and a library molecule with an affinity for a nanotube of <1E−9 M.

A5.4.5.7: A polysome-encoded peptide library, a medium comprising >50% organic solvent, a nanotube, and a library molecule with an affinity for a nanotube of <1E−10 M.

A5.4.5.8: A polysome-encoded peptide library, a medium comprising >50% organic solvent, a nanotube, and a library molecule with an affinity for a nanotube of <1E−12 M.

A5.4.6.1: A polysome-encoded peptide library, a medium comprising >50% organic solvent, a fullerene, and a library molecule with an affinity for a fullerene of <1E−2 M.

A5.4.6.2: A polysome-encoded peptide library, a medium comprising >50% organic solvent, a fullerene, and a library molecule with an affinity for a fullerene of <1E−4 M.

A5.4.6.3: A polysome-encoded peptide library, a medium comprising >50% organic solvent, a fullerene, and a library molecule with an affinity for a fullerene of <1E−6 M.

A5.4.6.4: A polysome-encoded peptide library, a medium comprising >50% organic solvent, a fullerene, and a library molecule with an affinity for a fullerene of <1E−7 M.

A5.4.6.5: A polysome-encoded peptide library, a medium comprising >50% organic solvent, a fullerene, and a library molecule with an affinity for a fullerene of <1E−8 M.

A5.4.6.6: A polysome-encoded peptide library, a medium comprising >50% organic solvent, a fullerene, and a library molecule with an affinity for a fullerene of <1E−9 M.

A5.4.6.7: A polysome-encoded peptide library, a medium comprising >50% organic solvent, a fullerene, and a library molecule with an affinity for a fullerene of <1E−10 M.

A5.4.6.8: A polysome-encoded peptide library, a medium comprising >50% organic solvent, a fullerene, and a library molecule with an affinity for a fullerene of <1E−12 M.

A5.4.7.1: A polysome-encoded peptide library, a medium comprising >50% organic solvent, a BNNT, and a library molecule with an affinity for a BNNT of <1E−2 M.

A5.4.7.2: A polysome-encoded peptide library, a medium comprising >50% organic solvent, a BNNT, and a library molecule with an affinity for a BNNT of <1E−4 M.

A5.4.7.3: A polysome-encoded peptide library, a medium comprising >50% organic solvent, a BNNT, and a library molecule with an affinity for a BNNT of <1E−6 M.

A5.4.7.4: A polysome-encoded peptide library, a medium comprising >50% organic solvent, a BNNT, and a library molecule with an affinity for a BNNT of <1E−7 M.

A5.4.7.5: A polysome-encoded peptide library, a medium comprising >50% organic solvent, a BNNT, and a library molecule with an affinity for a BNNT of <1E−8 M.

A5.4.7.6: A polysome-encoded peptide library, a medium comprising >50% organic solvent, a BNNT, and a library molecule with an affinity for a BNNT of <1E−9 M.

A5.4.7.7: A polysome-encoded peptide library, a medium comprising >50% organic solvent, a BNNT, and a library molecule with an affinity for a BNNT of <1E−10 M.

A5.4.7.8: A polysome-encoded peptide library, a medium comprising >50% organic solvent, a BNNT, and a library molecule with an affinity for a BNNT of <1E−12 M.

A5.5.1.1: A polysome-encoded peptide library, a medium comprising >50% organic solvent and a soluble polymer, a CNT, and a library molecule with an affinity for a CNT of <1E−2 M.

A5.5.1.2: A polysome-encoded peptide library, a medium comprising >50% organic solvent and a soluble polymer, a CNT, and a library molecule with an affinity for a CNT of <1E−4 M.

A5.5.1.3: A polysome-encoded peptide library, a medium comprising >50% organic solvent and a soluble polymer, a CNT, and a library molecule with an affinity for a CNT of <1E−6 M.

A5.5.1.4: A polysome-encoded peptide library, a medium comprising >50% organic solvent and a soluble polymer, a CNT, and a library molecule with an affinity for a CNT of <1E−7 M.

A5.5.1.5: A polysome-encoded peptide library, a medium comprising >50% organic solvent and a soluble polymer, a CNT, and a library molecule with an affinity for a CNT of <1E−8 M.

A5.5.1.6: A polysome-encoded peptide library, a medium comprising >50% organic solvent and a soluble polymer, a CNT, and a library molecule with an affinity for a CNT of <1E−9 M.

A5.5.1.7: A polysome-encoded peptide library, a medium comprising >50% organic solvent and a soluble polymer, a CNT, and a library molecule with an affinity for a CNT of <1E−10 M.

A5.5.1.8: A polysome-encoded peptide library, a medium comprising >50% organic solvent and a soluble polymer, a CNT, and a library molecule with an affinity for a CNT of <1E−12 M.

A5.5.2.1: A polysome-encoded peptide library, a medium comprising >50% organic solvent and a soluble polymer, a GS, and a library molecule with an affinity for a GS of <1E−2 M.

A5.5.2.2: A polysome-encoded peptide library, a medium comprising >50% organic solvent and a soluble polymer, a GS, and a library molecule with an affinity for a GS of <1E−4 M.

A5.5.2.3: A polysome-encoded peptide library, a medium comprising >50% organic solvent and a soluble polymer, a GS, and a library molecule with an affinity for a GS of <1E−6 M.

A5.5.2.4: A polysome-encoded peptide library, a medium comprising >50% organic solvent and a soluble polymer, a GS, and a library molecule with an affinity for a GS of <1E−7 M.

A5.5.2.5: A polysome-encoded peptide library, a medium comprising >50% organic solvent and a soluble polymer, a GS, and a library molecule with an affinity for a GS of <1E−8 M.

A5.5.2.6: A polysome-encoded peptide library, a medium comprising >50% organic solvent and a soluble polymer, a GS, and a library molecule with an affinity for a GS of <1E−9 M.

A5.5.2.7: A polysome-encoded peptide library, a medium comprising >50% organic solvent and a soluble polymer, a GS, and a library molecule with an affinity for a GS of <1E−10 M.

A5.5.2.8: A polysome-encoded peptide library, a medium comprising >50% organic solvent and a soluble polymer, a GS, and a library molecule with an affinity for a GS of <1E−12 M.

A5.5.3.1: A polysome-encoded peptide library, a medium comprising >50% organic solvent and a soluble polymer, a SWNT, and a library molecule with an affinity for a SWNT of <1E−2 M.

A5.5.3.2: A polysome-encoded peptide library, a medium comprising >50% organic solvent and a soluble polymer, a SWNT, and a library molecule with an affinity for a SWNT of <1E−4 M.

A5.5.3.3: A polysome-encoded peptide library, a medium comprising >50% organic solvent and a soluble polymer, a SWNT, and a library molecule with an affinity for a SWNT of <1E−6 M.

A5.5.3.4: A polysome-encoded peptide library, a medium comprising >50% organic solvent and a soluble polymer, a SWNT, and a library molecule with an affinity for a SWNT of <1E−7 M.

A5.5.3.5: A polysome-encoded peptide library, a medium comprising >50% organic solvent and a soluble polymer, a SWNT, and a library molecule with an affinity for a SWNT of <1E−8 M.

A5.5.3.6: A polysome-encoded peptide library, a medium comprising >50% organic solvent and a soluble polymer, a SWNT, and a library molecule with an affinity for a SWNT of <1E−9 M.

A5.5.3.7: A polysome-encoded peptide library, a medium comprising >50% organic solvent and a soluble polymer, a SWNT, and a library molecule with an affinity for a SWNT of <1E−10 M.

A5.5.3.8: A polysome-encoded peptide library, a medium comprising >50% organic solvent and a soluble polymer, a SWNT, and a library molecule with an affinity for a SWNT of <1E−12 M.

A5.5.4.1: A polysome-encoded peptide library, a medium comprising >50% organic solvent and a soluble polymer, a MWNT, and a library molecule with an affinity for a MWNT of <1E−2 M.

A5.5.4.2: A polysome-encoded peptide library, a medium comprising >50% organic solvent and a soluble polymer, a MWNT, and a library molecule with an affinity for a MWNT of <1E−4 M.

A5.5.4.3: A polysome-encoded peptide library, a medium comprising >50% organic solvent and a soluble polymer, a MWNT, and a library molecule with an affinity for a MWNT of <1E−6 M.

A5.5.4.4: A polysome-encoded peptide library, a medium comprising >50% organic solvent and a soluble polymer, a MWNT, and a library molecule with an affinity for a MWNT of <1E−7 M.

A5.5.4.5: A polysome-encoded peptide library, a medium comprising >50% organic solvent and a soluble polymer, a MWNT, and a library molecule with an affinity for a MWNT of <1E−8 M.

A5.5.4.6: A polysome-encoded peptide library, a medium comprising >50% organic solvent and a soluble polymer, a MWNT, and a library molecule with an affinity for a MWNT of <1E−9 M.

A5.5.4.7: A polysome-encoded peptide library, a medium comprising >50% organic solvent and a soluble polymer, a MWNT, and a library molecule with an affinity for a MWNT of <1E−10 M.

A5.5.4.8: A polysome-encoded peptide library, a medium comprising >50% organic solvent and a soluble polymer, a MWNT, and a library molecule with an affinity for a MWNT of <1E−12 M.

A5.5.5.1: A polysome-encoded peptide library, a medium comprising >50% organic solvent and a soluble polymer, a nanotube, and a library molecule with an affinity for a nanotube of <1E−2 M.

A5.5.5.2: A polysome-encoded peptide library, a medium comprising >50% organic solvent and a soluble polymer, a nanotube, and a library molecule with an affinity for a nanotube of <1E−4 M.

A5.5.5.3: A polysome-encoded peptide library, a medium comprising >50% organic solvent and a soluble polymer, a nanotube, and a library molecule with an affinity for a nanotube of <1E−6 M.

A5.5.5.4: A polysome-encoded peptide library, a medium comprising >50% organic solvent and a soluble polymer, a nanotube, and a library molecule with an affinity for a nanotube of <1E−7 M.

A5.5.5.5: A polysome-encoded peptide library, a medium comprising >50% organic solvent and a soluble polymer, a nanotube, and a library molecule with an affinity for a nanotube of <1E−8 M.

A5.5.5.6: A polysome-encoded peptide library, a medium comprising >50% organic solvent and a soluble polymer, a nanotube, and a library molecule with an affinity for a nanotube of <1E−9 M.

A5.5.5.7: A polysome-encoded peptide library, a medium comprising >50% organic solvent and a soluble polymer, a nanotube, and a library molecule with an affinity for a nanotube of <1E−10 M.

A5.5.5.8: A polysome-encoded peptide library, a medium comprising >50% organic solvent and a soluble polymer, a nanotube, and a library molecule with an affinity for a nanotube of <1E−12 M.

A5.5.6.1: A polysome-encoded peptide library, a medium comprising >50% organic solvent and a soluble polymer, a fullerene, and a library molecule with an affinity for a fullerene of <1E−2 M.

A5.5.6.2: A polysome-encoded peptide library, a medium comprising >50% organic solvent and a soluble polymer, a fullerene, and a library molecule with an affinity for a fullerene of <1E−4 M.

A5.5.6.3: A polysome-encoded peptide library, a medium comprising >50% organic solvent and a soluble polymer, a fullerene, and a library molecule with an affinity for a fullerene of <1E−6 M.

A5.5.6.4: A polysome-encoded peptide library, a medium comprising >50% organic solvent and a soluble polymer, a fullerene, and a library molecule with an affinity for a fullerene of <1E−7 M.

A5.5.6.5: A polysome-encoded peptide library, a medium comprising >50% organic solvent and a soluble polymer, a fullerene, and a library molecule with an affinity for a fullerene of <1E−8 M.

A5.5.6.6: A polysome-encoded peptide library, a medium comprising >50% organic solvent and a soluble polymer, a fullerene, and a library molecule with an affinity for a fullerene of <1E−9 M.

A5.5.6.7: A polysome-encoded peptide library, a medium comprising >50% organic solvent and a soluble polymer, a fullerene, and a library molecule with an affinity for a fullerene of <1E−10 M.

A5.5.6.8: A polysome-encoded peptide library, a medium comprising >50% organic solvent and a soluble polymer, a fullerene, and a library molecule with an affinity for a fullerene of <1E−12 M.

A5.5.7.1: A polysome-encoded peptide library, a medium comprising >50% organic solvent and a soluble polymer, a BNNT, and a library molecule with an affinity for a BNNT of <1E−2 M.

A5.5.7.2: A polysome-encoded peptide library, a medium comprising >50% organic solvent and a soluble polymer, a BNNT, and a library molecule with an affinity for a BNNT of <1E−4 M.

A5.5.7.3: A polysome-encoded peptide library, a medium comprising >50% organic solvent and a soluble polymer, a BNNT, and a library molecule with an affinity for a BNNT of <1E−6 M.

A5.5.7.4: A polysome-encoded peptide library, a medium comprising >50% organic solvent and a soluble polymer, a BNNT, and a library molecule with an affinity for a BNNT of <1E−7 M.

A5.5.7.5: A polysome-encoded peptide library, a medium comprising >50% organic solvent and a soluble polymer, a BNNT, and a library molecule with an affinity for a BNNT of <1E−8 M.

A5.5.7.6: A polysome-encoded peptide library, a medium comprising >50% organic solvent and a soluble polymer, a BNNT, and a library molecule with an affinity for a BNNT of <1E−9 M.

A5.5.7.7: A polysome-encoded peptide library, a medium comprising >50% organic solvent and a soluble polymer, a BNNT, and a library molecule with an affinity for a BNNT of <1E−10 M.

A5.5.7.8: A polysome-encoded peptide library, a medium comprising >50% organic solvent and a soluble polymer, a BNNT, and a library molecule with an affinity for a BNNT of <1E−12 M.

A5.6.1.1: A polysome-encoded peptide library, a medium comprising >80% organic solvent, a CNT, and a library molecule with an affinity for a CNT of <1E−2 M.

A5.6.1.2: A polysome-encoded peptide library, a medium comprising >80% organic solvent, a CNT, and a library molecule with an affinity for a CNT of <1E−4 M.

A5.6.1.3: A polysome-encoded peptide library, a medium comprising >80% organic solvent, a CNT, and a library molecule with an affinity for a CNT of <1E−6 M.

A5.6.1.4: A polysome-encoded peptide library, a medium comprising >80% organic solvent, a CNT, and a library molecule with an affinity for a CNT of <1E−7 M.

A5.6.1.5: A polysome-encoded peptide library, a medium comprising >80% organic solvent, a CNT, and a library molecule with an affinity for a CNT of <1E−8 M.

A5.6.1.6: A polysome-encoded peptide library, a medium comprising >80% organic solvent, a CNT, and a library molecule with an affinity for a CNT of <1E−9 M.

A5.6.1.7: A polysome-encoded peptide library, a medium comprising >80% organic solvent, a CNT, and a library molecule with an affinity for a CNT of <1E−10 M.

A5.6.1.8: A polysome-encoded peptide library, a medium comprising >80% organic solvent, a CNT, and a library molecule with an affinity for a CNT of <1E−12 M.

A5.6.2.1: A polysome-encoded peptide library, a medium comprising >80% organic solvent, a GS, and a library molecule with an affinity for a GS of <1E−2 M.

A5.6.2.2: A polysome-encoded peptide library, a medium comprising >80% organic solvent, a GS, and a library molecule with an affinity for a GS of <1E−4 M.

A5.6.2.3: A polysome-encoded peptide library, a medium comprising >80% organic solvent, a GS, and a library molecule with an affinity for a GS of <1E−6 M.

A5.6.2.4: A polysome-encoded peptide library, a medium comprising >80% organic solvent, a GS, and a library molecule with an affinity for a GS of <1E−7 M.

A5.6.2.5: A polysome-encoded peptide library, a medium comprising >80% organic solvent, a GS, and a library molecule with an affinity for a GS of <1E−8 M.

A5.6.2.6: A polysome-encoded peptide library, a medium comprising >80% organic solvent, a GS, and a library molecule with an affinity for a GS of <1E−9 M.

A5.6.2.7: A polysome-encoded peptide library, a medium comprising >80% organic solvent, a GS, and a library molecule with an affinity for a GS of <1E−10 M.

A5.6.2.8: A polysome-encoded peptide library, a medium comprising >80% organic solvent, a GS, and a library molecule with an affinity for a GS of <1E−12 M.

A5.6.3.1: A polysome-encoded peptide library, a medium comprising >80% organic solvent, a SWNT, and a library molecule with an affinity for a SWNT of <1E−2 M.

A5.6.3.2: A polysome-encoded peptide library, a medium comprising >80% organic solvent, a SWNT, and a library molecule with an affinity for a SWNT of <1E−4 M.

A5.6.3.3: A polysome-encoded peptide library, a medium comprising >80% organic solvent, a SWNT, and a library molecule with an affinity for a SWNT of <1E−6 M.

A5.6.3.4: A polysome-encoded peptide library, a medium comprising >80% organic solvent, a SWNT, and a library molecule with an affinity for a SWNT of <1E−7 M.

A5.6.3.5: A polysome-encoded peptide library, a medium comprising >80% organic solvent, a SWNT, and a library molecule with an affinity for a SWNT of <1E−8 M.

A5.6.3.6: A polysome-encoded peptide library, a medium comprising >80% organic solvent, a SWNT, and a library molecule with an affinity for a SWNT of <1E−9 M.

A5.6.3.7: A polysome-encoded peptide library, a medium comprising >80% organic solvent, a SWNT, and a library molecule with an affinity for a SWNT of <1E−10 M.

A5.6.3.8: A polysome-encoded peptide library, a medium comprising >80% organic solvent, a SWNT, and a library molecule with an affinity for a SWNT of <1E−12 M.

A5.6.4.1: A polysome-encoded peptide library, a medium comprising >80% organic solvent, a MWNT, and a library molecule with an affinity for a MWNT of <1E−2 M.

A5.6.4.2: A polysome-encoded peptide library, a medium comprising >80% organic solvent, a MWNT, and a library molecule with an affinity for a MWNT of <1E−4 M.

A5.6.4.3: A polysome-encoded peptide library, a medium comprising >80% organic solvent, a MWNT, and a library molecule with an affinity for a MWNT of <1E−6 M.

A5.6.4.4: A polysome-encoded peptide library, a medium comprising >80% organic solvent, a MWNT, and a library molecule with an affinity for a MWNT of <1E−7 M.

A5.6.4.5: A polysome-encoded peptide library, a medium comprising >80% organic solvent, a MWNT, and a library molecule with an affinity for a MWNT of <1E−8 M.

A5.6.4.6: A polysome-encoded peptide library, a medium comprising >80% organic solvent, a MWNT, and a library molecule with an affinity for a MWNT of <1E−9 M.

A5.6.4.7: A polysome-encoded peptide library, a medium comprising >80% organic solvent, a MWNT, and a library molecule with an affinity for a MWNT of <1E−10 M.

A5.6.4.8: A polysome-encoded peptide library, a medium comprising >80% organic solvent, a MWNT, and a library molecule with an affinity for a MWNT of <1E−12 M.

A5.6.5.1: A polysome-encoded peptide library, a medium comprising >80% organic solvent, a nanotube, and a library molecule with an affinity for a nanotube of <1E−2 M.

A5.6.5.2: A polysome-encoded peptide library, a medium comprising >80% organic solvent, a nanotube, and a library molecule with an affinity for a nanotube of <1E−4 M.

A5.6.5.3: A polysome-encoded peptide library, a medium comprising >80% organic solvent, a nanotube, and a library molecule with an affinity for a nanotube of <1E−6 M.

A5.6.5.4: A polysome-encoded peptide library, a medium comprising >80% organic solvent, a nanotube, and a library molecule with an affinity for a nanotube of <1E−7 M.

A5.6.5.5: A polysome-encoded peptide library, a medium comprising >80% organic solvent, a nanotube, and a library molecule with an affinity for a nanotube of <1E−8 M.

A5.6.5.6: A polysome-encoded peptide library, a medium comprising >80% organic solvent, a nanotube, and a library molecule with an affinity for a nanotube of <1E−9 M.

A5.6.5.7: A polysome-encoded peptide library, a medium comprising >80% organic solvent, a nanotube, and a library molecule with an affinity for a nanotube of <1E−10 M.

A5.6.5.8: A polysome-encoded peptide library, a medium comprising >80% organic solvent, a nanotube, and a library molecule with an affinity for a nanotube of <1E−12 M.

A5.6.6.1: A polysome-encoded peptide library, a medium comprising >80% organic solvent, a fullerene, and a library molecule with an affinity for a fullerene of <1E−2 M.

A5.6.6.2: A polysome-encoded peptide library, a medium comprising >80% organic solvent, a fullerene, and a library molecule with an affinity for a fullerene of <1E−4 M.

A5.6.6.3: A polysome-encoded peptide library, a medium comprising >80% organic solvent, a fullerene, and a library molecule with an affinity for a fullerene of <1E−6 M.

A5.6.6.4: A polysome-encoded peptide library, a medium comprising >80% organic solvent, a fullerene, and a library molecule with an affinity for a fullerene of <1E−7 M.

A5.6.6.5: A polysome-encoded peptide library, a medium comprising >80% organic solvent, a fullerene, and a library molecule with an affinity for a fullerene of <1E−8 M.

A5.6.6.6: A polysome-encoded peptide library, a medium comprising >80% organic solvent, a fullerene, and a library molecule with an affinity for a fullerene of <1E−9 M.

A5.6.6.7: A polysome-encoded peptide library, a medium comprising >80% organic solvent, a fullerene, and a library molecule with an affinity for a fullerene of <1E−10 M.

A5.6.6.8: A polysome-encoded peptide library, a medium comprising >80% organic solvent, a fullerene, and a library molecule with an affinity for a fullerene of <1E−12 M.

A5.6.7.1: A polysome-encoded peptide library, a medium comprising >80% organic solvent, a BNNT, and a library molecule with an affinity for a BNNT of <1E−2 M.

A5.6.7.2: A polysome-encoded peptide library, a medium comprising >80% organic solvent, a BNNT, and a library molecule with an affinity for a BNNT of <1E−4 M.

A5.6.7.3: A polysome-encoded peptide library, a medium comprising >80% organic solvent, a BNNT, and a library molecule with an affinity for a BNNT of <1E−6 M.

A5.6.7.4: A polysome-encoded peptide library, a medium comprising >80% organic solvent, a BNNT, and a library molecule with an affinity for a BNNT of <1E−7 M.

A5.6.7.5: A polysome-encoded peptide library, a medium comprising >80% organic solvent, a BNNT, and a library molecule with an affinity for a BNNT of <1E−8 M.

A5.6.7.6: A polysome-encoded peptide library, a medium comprising >80% organic solvent, a BNNT, and a library molecule with an affinity for a BNNT of <1E−9 M.

A5.6.7.7: A polysome-encoded peptide library, a medium comprising >80% organic solvent, a BNNT, and a library molecule with an affinity for a BNNT of <1E−10 M.

A5.6.7.8: A polysome-encoded peptide library, a medium comprising >80% organic solvent, a BNNT, and a library molecule with an affinity for a BNNT of <1E−12 M.

A5.7.1.1: A polysome-encoded peptide library, a medium comprising >80% organic solvent and a soluble polymer, a CNT, and a library molecule with an affinity for a CNT of <1E−2 M.

A5.7.1.2: A polysome-encoded peptide library, a medium comprising >80% organic solvent and a soluble polymer, a CNT, and a library molecule with an affinity for a CNT of <1E−4 M.

A5.7.1.3: A polysome-encoded peptide library, a medium comprising >80% organic solvent and a soluble polymer, a CNT, and a library molecule with an affinity for a CNT of <1E−6 M.

A5.7.1.4: A polysome-encoded peptide library, a medium comprising >80% organic solvent and a soluble polymer, a CNT, and a library molecule with an affinity for a CNT of <1E−7 M.

A5.7.1.5: A polysome-encoded peptide library, a medium comprising >80% organic solvent and a soluble polymer, a CNT, and a library molecule with an affinity for a CNT of <1E−8 M.

A5.7.1.6: A polysome-encoded peptide library, a medium comprising >80% organic solvent and a soluble polymer, a CNT, and a library molecule with an affinity for a CNT of <1E−9 M.

A5.7.1.7: A polysome-encoded peptide library, a medium comprising >80% organic solvent and a soluble polymer, a CNT, and a library molecule with an affinity for a CNT of <1E−10 M.

A5.7.1.8: A polysome-encoded peptide library, a medium comprising >80% organic solvent and a soluble polymer, a CNT, and a library molecule with an affinity for a CNT of <1E−12 M.

A5.7.2.1: A polysome-encoded peptide library, a medium comprising >80% organic solvent and a soluble polymer, a GS, and a library molecule with an affinity for a GS of <1E−2 M.

A5.7.2.2: A polysome-encoded peptide library, a medium comprising >80% organic solvent and a soluble polymer, a GS, and a library molecule with an affinity for a GS of <1E−4 M.

A5.7.2.3: A polysome-encoded peptide library, a medium comprising >80% organic solvent and a soluble polymer, a GS, and a library molecule with an affinity for a GS of <1E−6 M.

A5.7.2.4: A polysome-encoded peptide library, a medium comprising >80% organic solvent and a soluble polymer, a GS, and a library molecule with an affinity for a GS of <1E−7 M.

A5.7.2.5: A polysome-encoded peptide library, a medium comprising >80% organic solvent and a soluble polymer, a GS, and a library molecule with an affinity for a GS of <1E−8 M.

A5.7.2.6: A polysome-encoded peptide library, a medium comprising >80% organic solvent and a soluble polymer, a GS, and a library molecule with an affinity for a GS of <1E−9 M.

A5.7.2.7: A polysome-encoded peptide library, a medium comprising >80% organic solvent and a soluble polymer, a GS, and a library molecule with an affinity for a GS of <1E−10 M.

A5.7.2.8: A polysome-encoded peptide library, a medium comprising >80% organic solvent and a soluble polymer, a GS, and a library molecule with an affinity for a GS of <1E−12 M.

A5.7.3.1: A polysome-encoded peptide library, a medium comprising >80% organic solvent and a soluble polymer, a SWNT, and a library molecule with an affinity for a SWNT of <1E−2 M.

A5.7.3.2: A polysome-encoded peptide library, a medium comprising >80% organic solvent and a soluble polymer, a SWNT, and a library molecule with an affinity for a SWNT of <1E−4 M.

A5.7.3.3: A polysome-encoded peptide library, a medium comprising >80% organic solvent and a soluble polymer, a SWNT, and a library molecule with an affinity for a SWNT of <1E−6 M.

A5.7.3.4: A polysome-encoded peptide library, a medium comprising >80% organic solvent and a soluble polymer, a SWNT, and a library molecule with an affinity for a SWNT of <1E−7 M.

A5.7.3.5: A polysome-encoded peptide library, a medium comprising >80% organic solvent and a soluble polymer, a SWNT, and a library molecule with an affinity for a SWNT of <1E−8 M.

A5.7.3.6: A polysome-encoded peptide library, a medium comprising >80% organic solvent and a soluble polymer, a SWNT, and a library molecule with an affinity for a SWNT of <1E−9 M.

A5.7.3.7: A polysome-encoded peptide library, a medium comprising >80% organic solvent and a soluble polymer, a SWNT, and a library molecule with an affinity for a SWNT of <1E−10 M.

A5.7.3.8: A polysome-encoded peptide library, a medium comprising >80% organic solvent and a soluble polymer, a SWNT, and a library molecule with an affinity for a SWNT of <1E−12 M.

A5.7.4.1: A polysome-encoded peptide library, a medium comprising >80% organic solvent and a soluble polymer, a MWNT, and a library molecule with an affinity for a MWNT of <1E−2 M.

A5.7.4.2: A polysome-encoded peptide library, a medium comprising >80% organic solvent and a soluble polymer, a MWNT, and a library molecule with an affinity for a MWNT of <1E−4 M.

A5.7.4.3: A polysome-encoded peptide library, a medium comprising >80% organic solvent and a soluble polymer, a MWNT, and a library molecule with an affinity for a MWNT of <1E−6 M.

A5.7.4.4: A polysome-encoded peptide library, a medium comprising >80% organic solvent and a soluble polymer, a MWNT, and a library molecule with an affinity for a MWNT of <1E−7 M.

A5.7.4.5: A polysome-encoded peptide library, a medium comprising >80% organic solvent and a soluble polymer, a MWNT, and a library molecule with an affinity for a MWNT of <1E−8 M.

A5.7.4.6: A polysome-encoded peptide library, a medium comprising >80% organic solvent and a soluble polymer, a MWNT, and a library molecule with an affinity for a MWNT of <1E−9 M.

A5.7.4.7: A polysome-encoded peptide library, a medium comprising >80% organic solvent and a soluble polymer, a MWNT, and a library molecule with an affinity for a MWNT of <1E−10 M.

A5.7.4.8: A polysome-encoded peptide library, a medium comprising >80% organic solvent and a soluble polymer, a MWNT, and a library molecule with an affinity for a MWNT of <1E−12 M.

A5.7.5.1: A polysome-encoded peptide library, a medium comprising >80% organic solvent and a soluble polymer, a nanotube, and a library molecule with an affinity for a nanotube of <1E−2 M.

A5.7.5.2: A polysome-encoded peptide library, a medium comprising >80% organic solvent and a soluble polymer, a nanotube, and a library molecule with an affinity for a nanotube of <1E−4 M.

A5.7.5.3: A polysome-encoded peptide library, a medium comprising >80% organic solvent and a soluble polymer, a nanotube, and a library molecule with an affinity for a nanotube of <1E−6 M.

A5.7.5.4: A polysome-encoded peptide library, a medium comprising >80% organic solvent and a soluble polymer, a nanotube, and a library molecule with an affinity for a nanotube of <1E−7 M.

A5.7.5.5: A polysome-encoded peptide library, a medium comprising >80% organic solvent and a soluble polymer, a nanotube, and a library molecule with an affinity for a nanotube of <1E−8 M.

A5.7.5.6: A polysome-encoded peptide library, a medium comprising >80% organic solvent and a soluble polymer, a nanotube, and a library molecule with an affinity for a nanotube of <1E−9 M.

A5.7.5.7: A polysome-encoded peptide library, a medium comprising >80% organic solvent and a soluble polymer, a nanotube, and a library molecule with an affinity for a nanotube of <1E−10 M.

A5.7.5.8: A polysome-encoded peptide library, a medium comprising >80% organic solvent and a soluble polymer, a nanotube, and a library molecule with an affinity for a nanotube of <1E−12 M.

A5.7.6.1: A polysome-encoded peptide library, a medium comprising >80% organic solvent and a soluble polymer, a fullerene, and a library molecule with an affinity for a fullerene of <1E−2 M.

A5.7.6.2: A polysome-encoded peptide library, a medium comprising >80% organic solvent and a soluble polymer, a fullerene, and a library molecule with an affinity for a fullerene of <1E−4 M.

A5.7.6.3: A polysome-encoded peptide library, a medium comprising >80% organic solvent and a soluble polymer, a fullerene, and a library molecule with an affinity for a fullerene of <1E−6 M.

A5.7.6.4: A polysome-encoded peptide library, a medium comprising >80% organic solvent and a soluble polymer, a fullerene, and a library molecule with an affinity for a fullerene of <1E−7 M.

A5.7.6.5: A polysome-encoded peptide library, a medium comprising >80% organic solvent and a soluble polymer, a fullerene, and a library molecule with an affinity for a fullerene of <1E−8 M.

A5.7.6.6: A polysome-encoded peptide library, a medium comprising >80% organic solvent and a soluble polymer, a fullerene, and a library molecule with an affinity for a fullerene of <1E−9 M.

A5.7.6.7: A polysome-encoded peptide library, a medium comprising >80% organic solvent and a soluble polymer, a fullerene, and a library molecule with an affinity for a fullerene of <1E−10 M.

A5.7.6.8: A polysome-encoded peptide library, a medium comprising >80% organic solvent and a soluble polymer, a fullerene, and a library molecule with an affinity for a fullerene of <1E−12 M.

A5.7.7.1: A polysome-encoded peptide library, a medium comprising >80% organic solvent and a soluble polymer, a BNNT, and a library molecule with an affinity for a BNNT of <1E−2 M.

A5.7.7.2: A polysome-encoded peptide library, a medium comprising >80% organic solvent and a soluble polymer, a BNNT, and a library molecule with an affinity for a BNNT of <1E−4 M.

A5.7.7.3: A polysome-encoded peptide library, a medium comprising >80% organic solvent and a soluble polymer, a BNNT, and a library molecule with an affinity for a BNNT of <1E−6 M.

A5.7.7.4: A polysome-encoded peptide library, a medium comprising >80% organic solvent and a soluble polymer, a BNNT, and a library molecule with an affinity for a BNNT of <1E−7 M.

A5.7.7.5: A polysome-encoded peptide library, a medium comprising >80% organic solvent and a soluble polymer, a BNNT, and a library molecule with an affinity for a BNNT of <1E−8 M.

A5.7.7.6: A polysome-encoded peptide library, a medium comprising >80% organic solvent and a soluble polymer, a BNNT, and a library molecule with an affinity for a BNNT of <1E−9 M.

A5.7.7.7: A polysome-encoded peptide library, a medium comprising >80% organic solvent and a soluble polymer, a BNNT, and a library molecule with an affinity for a BNNT of <1E−10 M.

A5.7.7.8: A polysome-encoded peptide library, a medium comprising >80% organic solvent and a soluble polymer, a BNNT, and a library molecule with an affinity for a BNNT of <1E−12 M.

A5.8.1.1: A polysome-encoded peptide library, a medium comprising >95% organic solvent, a CNT, and a library molecule with an affinity for a CNT of <1E−2 M.

A5.8.1.2: A polysome-encoded peptide library, a medium comprising >95% organic solvent, a CNT, and a library molecule with an affinity for a CNT of <1E−4 M.

A5.8.1.3: A polysome-encoded peptide library, a medium comprising >95% organic solvent, a CNT, and a library molecule with an affinity for a CNT of <1E−6 M.

A5.8.1.4: A polysome-encoded peptide library, a medium comprising >95% organic solvent, a CNT, and a library molecule with an affinity for a CNT of <1E−7 M.

A5.8.1.5: A polysome-encoded peptide library, a medium comprising >95% organic solvent, a CNT, and a library molecule with an affinity for a CNT of <1E−8 M.

A5.8.1.6: A polysome-encoded peptide library, a medium comprising >95% organic solvent, a CNT, and a library molecule with an affinity for a CNT of <1E−9 M.

A5.8.1.7: A polysome-encoded peptide library, a medium comprising >95% organic solvent, a CNT, and a library molecule with an affinity for a CNT of <1E−10 M.

A5.8.1.8: A polysome-encoded peptide library, a medium comprising >95% organic solvent, a CNT, and a library molecule with an affinity for a CNT of <1E−12 M.

A5.8.2.1: A polysome-encoded peptide library, a medium comprising >95% organic solvent, a GS, and a library molecule with an affinity for a GS of <1E−2 M.

A5.8.2.2: A polysome-encoded peptide library, a medium comprising >95% organic solvent, a GS, and a library molecule with an affinity for a GS of <1E−4 M.

A5.8.2.3: A polysome-encoded peptide library, a medium comprising >95% organic solvent, a GS, and a library molecule with an affinity for a GS of <1E−6 M.

A5.8.2.4: A polysome-encoded peptide library, a medium comprising >95% organic solvent, a GS, and a library molecule with an affinity for a GS of <1E−7 M.

A5.8.2.5: A polysome-encoded peptide library, a medium comprising >95% organic solvent, a GS, and a library molecule with an affinity for a GS of <1E−8 M.

A5.8.2.6: A polysome-encoded peptide library, a medium comprising >95% organic solvent, a GS, and a library molecule with an affinity for a GS of <1E−9 M.

A5.8.2.7: A polysome-encoded peptide library, a medium comprising >95% organic solvent, a GS, and a library molecule with an affinity for a GS of <1E−10 M.

A5.8.2.8: A polysome-encoded peptide library, a medium comprising >95% organic solvent, a GS, and a library molecule with an affinity for a GS of <1E−12 M.

A5.8.3.1: A polysome-encoded peptide library, a medium comprising >95% organic solvent, a SWNT, and a library molecule with an affinity for a SWNT of <1E−2 M.

A5.8.3.2: A polysome-encoded peptide library, a medium comprising >95% organic solvent, a SWNT, and a library molecule with an affinity for a SWNT of <1E−4 M.

A5.8.3.3: A polysome-encoded peptide library, a medium comprising >95% organic solvent, a SWNT, and a library molecule with an affinity for a SWNT of <1E−6 M.

A5.8.3.4: A polysome-encoded peptide library, a medium comprising >95% organic solvent, a SWNT, and a library molecule with an affinity for a SWNT of <1E−7 M.

A5.8.3.5: A polysome-encoded peptide library, a medium comprising >95% organic solvent, a SWNT, and a library molecule with an affinity for a SWNT of <1E−8 M.

A5.8.3.6: A polysome-encoded peptide library, a medium comprising >95% organic solvent, a SWNT, and a library molecule with an affinity for a SWNT of <1E−9 M.

A5.8.3.7: A polysome-encoded peptide library, a medium comprising >95% organic solvent, a SWNT, and a library molecule with an affinity for a SWNT of <1E−10 M.

A5.8.3.8: A polysome-encoded peptide library, a medium comprising >95% organic solvent, a SWNT, and a library molecule with an affinity for a SWNT of <1E−12 M.

A5.8.4.1: A polysome-encoded peptide library, a medium comprising >95% organic solvent, a MWNT, and a library molecule with an affinity for a MWNT of <1E−2 M.

A5.8.4.2: A polysome-encoded peptide library, a medium comprising >95% organic solvent, a MWNT, and a library molecule with an affinity for a MWNT of <1E−4 M.

A5.8.4.3: A polysome-encoded peptide library, a medium comprising >95% organic solvent, a MWNT, and a library molecule with an affinity for a MWNT of <1E−6 M.

A5.8.4.4: A polysome-encoded peptide library, a medium comprising >95% organic solvent, a MWNT, and a library molecule with an affinity for a MWNT of <1E−7 M.

A5.8.4.5: A polysome-encoded peptide library, a medium comprising >95% organic solvent, a MWNT, and a library molecule with an affinity for a MWNT of <1E−8 M.

A5.8.4.6: A polysome-encoded peptide library, a medium comprising >95% organic solvent, a MWNT, and a library molecule with an affinity for a MWNT of <1E−9 M.

A5.8.4.7: A polysome-encoded peptide library, a medium comprising >95% organic solvent, a MWNT, and a library molecule with an affinity for a MWNT of <1E−10 M.

A5.8.4.8: A polysome-encoded peptide library, a medium comprising >95% organic solvent, a MWNT, and a library molecule with an affinity for a MWNT of <1E−12 M.

A5.8.5.1: A polysome-encoded peptide library, a medium comprising >95% organic solvent, a nanotube, and a library molecule with an affinity for a nanotube of <1E−2 M.

A5.8.5.2: A polysome-encoded peptide library, a medium comprising >95% organic solvent, a nanotube, and a library molecule with an affinity for a nanotube of <1E−4 M.

A5.8.5.3: A polysome-encoded peptide library, a medium comprising >95% organic solvent, a nanotube, and a library molecule with an affinity for a nanotube of <1E−6 M.

A5.8.5.4: A polysome-encoded peptide library, a medium comprising >95% organic solvent, a nanotube, and a library molecule with an affinity for a nanotube of <1E−7 M.

A5.8.5.5: A polysome-encoded peptide library, a medium comprising >95% organic solvent, a nanotube, and a library molecule with an affinity for a nanotube of <1E−8 M.

A5.8.5.6: A polysome-encoded peptide library, a medium comprising >95% organic solvent, a nanotube, and a library molecule with an affinity for a nanotube of <1E−9 M.

A5.8.5.7: A polysome-encoded peptide library, a medium comprising >95% organic solvent, a nanotube, and a library molecule with an affinity for a nanotube of <1E−10 M.

A5.8.5.8: A polysome-encoded peptide library, a medium comprising >95% organic solvent, a nanotube, and a library molecule with an affinity for a nanotube of <1E−12 M.

A5.8.6.1: A polysome-encoded peptide library, a medium comprising >95% organic solvent, a fullerene, and a library molecule with an affinity for a fullerene of <1E−2 M.

A5.8.6.2: A polysome-encoded peptide library, a medium comprising >95% organic solvent, a fullerene, and a library molecule with an affinity for a fullerene of <1E−4 M.

A5.8.6.3: A polysome-encoded peptide library, a medium comprising >95% organic solvent, a fullerene, and a library molecule with an affinity for a fullerene of <1E−6 M.

A5.8.6.4: A polysome-encoded peptide library, a medium comprising >95% organic solvent, a fullerene, and a library molecule with an affinity for a fullerene of <1E−7 M.

A5.8.6.5: A polysome-encoded peptide library, a medium comprising >95% organic solvent, a fullerene, and a library molecule with an affinity for a fullerene of <1E−8 M.

A5.8.6.6: A polysome-encoded peptide library, a medium comprising >95% organic solvent, a fullerene, and a library molecule with an affinity for a fullerene of <1E−9 M.

A5.8.6.7: A polysome-encoded peptide library, a medium comprising >95% organic solvent, a fullerene, and a library molecule with an affinity for a fullerene of <1E−10 M.

A5.8.6.8: A polysome-encoded peptide library, a medium comprising >95% organic solvent, a fullerene, and a library molecule with an affinity for a fullerene of <1E−12 M.

A5.8.7.1: A polysome-encoded peptide library, a medium comprising >95% organic solvent, a BNNT, and a library molecule with an affinity for a BNNT of <1E−2 M.

A5.8.7.2: A polysome-encoded peptide library, a medium comprising >95% organic solvent, a BNNT, and a library molecule with an affinity for a BNNT of <1E−4 M.

A5.8.7.3: A polysome-encoded peptide library, a medium comprising >95% organic solvent, a BNNT, and a library molecule with an affinity for a BNNT of <1E−6 M.

A5.8.7.4: A polysome-encoded peptide library, a medium comprising >95% organic solvent, a BNNT, and a library molecule with an affinity for a BNNT of <1E−7 M.

A5.8.7.5: A polysome-encoded peptide library, a medium comprising >95% organic solvent, a BNNT, and a library molecule with an affinity for a BNNT of <1E−8 M.

A5.8.7.6: A polysome-encoded peptide library, a medium comprising >95% organic solvent, a BNNT, and a library molecule with an affinity for a BNNT of <1E−9 M.

A5.8.7.7: A polysome-encoded peptide library, a medium comprising >95% organic solvent, a BNNT, and a library molecule with an affinity for a BNNT of <1E−10 M.

A5.8.7.8: A polysome-encoded peptide library, a medium comprising >95% organic solvent, a BNNT, and a library molecule with an affinity for a BNNT of <1E−12 M.

A5.9.1.1: A polysome-encoded peptide library, a medium comprising >95% organic solvent and a soluble polymer, a CNT, and a library molecule with an affinity for a CNT of <1E−2 M.

A5.9.1.2: A polysome-encoded peptide library, a medium comprising >95% organic solvent and a soluble polymer, a CNT, and a library molecule with an affinity for a CNT of <1E−4 M.

A5.9.1.3: A polysome-encoded peptide library, a medium comprising >95% organic solvent and a soluble polymer, a CNT, and a library molecule with an affinity for a CNT of <1E−6 M.

A5.9.1.4: A polysome-encoded peptide library, a medium comprising >95% organic solvent and a soluble polymer, a CNT, and a library molecule with an affinity for a CNT of <1E−7 M.

A5.9.1.5: A polysome-encoded peptide library, a medium comprising >95% organic solvent and a soluble polymer, a CNT, and a library molecule with an affinity for a CNT of <1E−8 M.

A5.9.1.6: A polysome-encoded peptide library, a medium comprising >95% organic solvent and a soluble polymer, a CNT, and a library molecule with an affinity for a CNT of <1E−9 M.

A5.9.1.7: A polysome-encoded peptide library, a medium comprising >95% organic solvent and a soluble polymer, a CNT, and a library molecule with an affinity for a CNT of <1E−10 M.

A5.9.1.8: A polysome-encoded peptide library, a medium comprising >95% organic solvent and a soluble polymer, a CNT, and a library molecule with an affinity for a CNT of <1E−12 M.

A5.9.2.1: A polysome-encoded peptide library, a medium comprising >95% organic solvent and a soluble polymer, a GS, and a library molecule with an affinity for a GS of <1E−2 M.

A5.9.2.2: A polysome-encoded peptide library, a medium comprising >95% organic solvent and a soluble polymer, a GS, and a library molecule with an affinity for a GS of <1E−4 M.

A5.9.2.3: A polysome-encoded peptide library, a medium comprising >95% organic solvent and a soluble polymer, a GS, and a library molecule with an affinity for a GS of <1E−6 M.

A5.9.2.4: A polysome-encoded peptide library, a medium comprising >95% organic solvent and a soluble polymer, a GS, and a library molecule with an affinity for a GS of <1E−7 M.

A5.9.2.5: A polysome-encoded peptide library, a medium comprising >95% organic solvent and a soluble polymer, a GS, and a library molecule with an affinity for a GS of <1E−8 M.

A5.9.2.6: A polysome-encoded peptide library, a medium comprising >95% organic solvent and a soluble polymer, a GS, and a library molecule with an affinity for a GS of <1E−9 M.

A5.9.2.7: A polysome-encoded peptide library, a medium comprising >95% organic solvent and a soluble polymer, a GS, and a library molecule with an affinity for a GS of <1E−10 M.

A5.9.2.8: A polysome-encoded peptide library, a medium comprising >95% organic solvent and a soluble polymer, a GS, and a library molecule with an affinity for a GS of <1E−12 M.

A5.9.3.1: A polysome-encoded peptide library, a medium comprising >95% organic solvent and a soluble polymer, a SWNT, and a library molecule with an affinity for a SWNT of <1E−2 M.

A5.9.3.2: A polysome-encoded peptide library, a medium comprising >95% organic solvent and a soluble polymer, a SWNT, and a library molecule with an affinity for a SWNT of <1E−4 M.

A5.9.3.3: A polysome-encoded peptide library, a medium comprising >95% organic solvent and a soluble polymer, a SWNT, and a library molecule with an affinity for a SWNT of <1E−6 M.

A5.9.3.4: A polysome-encoded peptide library, a medium comprising >95% organic solvent and a soluble polymer, a SWNT, and a library molecule with an affinity for a SWNT of <1E−7 M.

A5.9.3.5: A polysome-encoded peptide library, a medium comprising >95% organic solvent and a soluble polymer, a SWNT, and a library molecule with an affinity for a SWNT of <1E−8 M.

A5.9.3.6: A polysome-encoded peptide library, a medium comprising >95% organic solvent and a soluble polymer, a SWNT, and a library molecule with an affinity for a SWNT of <1E−9 M.

A5.9.3.7: A polysome-encoded peptide library, a medium comprising >95% organic solvent and a soluble polymer, a SWNT, and a library molecule with an affinity for a SWNT of <1E−10 M.

A5.9.3.8: A polysome-encoded peptide library, a medium comprising >95% organic solvent and a soluble polymer, a SWNT, and a library molecule with an affinity for a SWNT of <1E−12 M.

A5.9.4.1: A polysome-encoded peptide library, a medium comprising >95% organic solvent and a soluble polymer, a MWNT, and a library molecule with an affinity for a MWNT of <1E−2 M.

A5.9.4.2: A polysome-encoded peptide library, a medium comprising >95% organic solvent and a soluble polymer, a MWNT, and a library molecule with an affinity for a MWNT of <1E−4 M.

A5.9.4.3: A polysome-encoded peptide library, a medium comprising >95% organic solvent and a soluble polymer, a MWNT, and a library molecule with an affinity for a MWNT of <1E−6 M.

A5.9.4.4: A polysome-encoded peptide library, a medium comprising >95% organic solvent and a soluble polymer, a MWNT, and a library molecule with an affinity for a MWNT of <1E−7 M.

A5.9.4.5: A polysome-encoded peptide library, a medium comprising >95% organic solvent and a soluble polymer, a MWNT, and a library molecule with an affinity for a MWNT of <1E−8 M.

A5.9.4.6: A polysome-encoded peptide library, a medium comprising >95% organic solvent and a soluble polymer, a MWNT, and a library molecule with an affinity for a MWNT of <1E−9 M.

A5.9.4.7: A polysome-encoded peptide library, a medium comprising >95% organic solvent and a soluble polymer, a MWNT, and a library molecule with an affinity for a MWNT of <1E−10 M.

A5.9.4.8: A polysome-encoded peptide library, a medium comprising >95% organic solvent and a soluble polymer, a MWNT, and a library molecule with an affinity for a MWNT of <1E−12 M.

A5.9.5.1: A polysome-encoded peptide library, a medium comprising >95% organic solvent and a soluble polymer, a nanotube, and a library molecule with an affinity for a nanotube of <1E−2 M.

A5.9.5.2: A polysome-encoded peptide library, a medium comprising >95% organic solvent and a soluble polymer, a nanotube, and a library molecule with an affinity for a nanotube of <1E−4 M.

A5.9.5.3: A polysome-encoded peptide library, a medium comprising >95% organic solvent and a soluble polymer, a nanotube, and a library molecule with an affinity for a nanotube of <1E−6 M.

A5.9.5.4: A polysome-encoded peptide library, a medium comprising >95% organic solvent and a soluble polymer, a nanotube, and a library molecule with an affinity for a nanotube of <1E−7 M.

A5.9.5.5: A polysome-encoded peptide library, a medium comprising >95% organic solvent and a soluble polymer, a nanotube, and a library molecule with an affinity for a nanotube of <1E−8 M.

A5.9.5.6: A polysome-encoded peptide library, a medium comprising >95% organic solvent and a soluble polymer, a nanotube, and a library molecule with an affinity for a nanotube of <1E−9 M.

A5.9.5.7: A polysome-encoded peptide library, a medium comprising >95% organic solvent and a soluble polymer, a nanotube, and a library molecule with an affinity for a nanotube of <1E−10 M.

A5.9.5.8: A polysome-encoded peptide library, a medium comprising >95% organic solvent and a soluble polymer, a nanotube, and a library molecule with an affinity for a nanotube of <1E−12 M.

A5.9.6.1: A polysome-encoded peptide library, a medium comprising >95% organic solvent and a soluble polymer, a fullerene, and a library molecule with an affinity for a fullerene of <1E−2 M.

A5.9.6.2: A polysome-encoded peptide library, a medium comprising >95% organic solvent and a soluble polymer, a fullerene, and a library molecule with an affinity for a fullerene of <1E−4 M.

A5.9.6.3: A polysome-encoded peptide library, a medium comprising >95% organic solvent and a soluble polymer, a fullerene, and a library molecule with an affinity for a fullerene of <1E−6 M.

A5.9.6.4: A polysome-encoded peptide library, a medium comprising >95% organic solvent and a soluble polymer, a fullerene, and a library molecule with an affinity for a fullerene of <1E−7 M.

A5.9.6.5: A polysome-encoded peptide library, a medium comprising >95% organic solvent and a soluble polymer, a fullerene, and a library molecule with an affinity for a fullerene of <1E−8 M.

A5.9.6.6: A polysome-encoded peptide library, a medium comprising >95% organic solvent and a soluble polymer, a fullerene, and a library molecule with an affinity for a fullerene of <1E−9 M.

A5.9.6.7: A polysome-encoded peptide library, a medium comprising >95% organic solvent and a soluble polymer, a fullerene, and a library molecule with an affinity for a fullerene of <1E−10 M.

A5.9.6.8: A polysome-encoded peptide library, a medium comprising >95% organic solvent and a soluble polymer, a fullerene, and a library molecule with an affinity for a fullerene of <1E−12 M.

A5.9.7.1: A polysome-encoded peptide library, a medium comprising >95% organic solvent and a soluble polymer, a BNNT, and a library molecule with an affinity for a BNNT of <1E−2 M.

A5.9.7.2: A polysome-encoded peptide library, a medium comprising >95% organic solvent and a soluble polymer, a BNNT, and a library molecule with an affinity for a BNNT of <1E−4 M.

A5.9.7.3: A polysome-encoded peptide library, a medium comprising >95% organic solvent and a soluble polymer, a BNNT, and a library molecule with an affinity for a BNNT of <1E−6 M.

A5.9.7.4: A polysome-encoded peptide library, a medium comprising >95% organic solvent and a soluble polymer, a BNNT, and a library molecule with an affinity for a BNNT of <1E−7 M.

A5.9.7.5: A polysome-encoded peptide library, a medium comprising >95% organic solvent and a soluble polymer, a BNNT, and a library molecule with an affinity for a BNNT of <1E−8 M.

A5.9.7.6: A polysome-encoded peptide library, a medium comprising >95% organic solvent and a soluble polymer, a BNNT, and a library molecule with an affinity for a BNNT of <1E−9 M.

A5.9.7.7: A polysome-encoded peptide library, a medium comprising >95% organic solvent and a soluble polymer, a BNNT, and a library molecule with an affinity for a BNNT of <1E−10 M.

A5.9.7.8: A polysome-encoded peptide library, a medium comprising >95% organic solvent and a soluble polymer, a BNNT, and a library molecule with an affinity for a BNNT of <1E−12 M.

A6.1.1.1: A cell-encoded peptide library, a medium comprising an organic solvent, a CNT, and a library molecule with an affinity for a CNT of <1E−2 M.

A6.1.1.2: A cell-encoded peptide library, a medium comprising an organic solvent, a CNT, and a library molecule with an affinity for a CNT of <1E−4 M.

A6.1.1.3: A cell-encoded peptide library, a medium comprising an organic solvent, a CNT, and a library molecule with an affinity for a CNT of <1E−6 M.

A6.1.1.4: A cell-encoded peptide library, a medium comprising an organic solvent, a CNT, and a library molecule with an affinity for a CNT of <1E−7 M.

A6.1.1.5: A cell-encoded peptide library, a medium comprising an organic solvent, a CNT, and a library molecule with an affinity for a CNT of <1E−8 M.

A6.1.1.6: A cell-encoded peptide library, a medium comprising an organic solvent, a CNT, and a library molecule with an affinity for a CNT of <1E−9 M.

A6.1.1.7: A cell-encoded peptide library, a medium comprising an organic solvent, a CNT, and a library molecule with an affinity for a CNT of <1E−10 M.

A6.1.1.8: A cell-encoded peptide library, a medium comprising an organic solvent, a CNT, and a library molecule with an affinity for a CNT of <1E−12 M.

A6.1.2.1: A cell-encoded peptide library, a medium comprising an organic solvent, a GS, and a library molecule with an affinity for a GS of <1E−2 M.

A6.1.2.2: A cell-encoded peptide library, a medium comprising an organic solvent, a GS, and a library molecule with an affinity for a GS of <1E−4 M.

A6.1.2.3: A cell-encoded peptide library, a medium comprising an organic solvent, a GS, and a library molecule with an affinity for a GS of <1E−6 M.

A6.1.2.4: A cell-encoded peptide library, a medium comprising an organic solvent, a GS, and a library molecule with an affinity for a GS of <1E−7 M.

A6.1.2.5: A cell-encoded peptide library, a medium comprising an organic solvent, a GS, and a library molecule with an affinity for a GS of <1E−8 M.

A6.1.2.6: A cell-encoded peptide library, a medium comprising an organic solvent, a GS, and a library molecule with an affinity for a GS of <1E−9 M.

A6.1.2.7: A cell-encoded peptide library, a medium comprising an organic solvent, a GS, and a library molecule with an affinity for a GS of <1E−10 M.

A6.1.2.8: A cell-encoded peptide library, a medium comprising an organic solvent, a GS, and a library molecule with an affinity for a GS of <1E−12 M.

A6.1.3.1: A cell-encoded peptide library, a medium comprising an organic solvent, a SWNT, and a library molecule with an affinity for a SWNT of <1E−2 M.

A6.1.3.2: A cell-encoded peptide library, a medium comprising an organic solvent, a SWNT, and a library molecule with an affinity for a SWNT of <1E−4 M.

A6.1.3.3: A cell-encoded peptide library, a medium comprising an organic solvent, a SWNT, and a library molecule with an affinity for a SWNT of <1E−6 M.

A6.1.3.4: A cell-encoded peptide library, a medium comprising an organic solvent, a SWNT, and a library molecule with an affinity for a SWNT of <1E−7 M.

A6.1.3.5: A cell-encoded peptide library, a medium comprising an organic solvent, a SWNT, and a library molecule with an affinity for a SWNT of <1E−8 M.

A6.1.3.6: A cell-encoded peptide library, a medium comprising an organic solvent, a SWNT, and a library molecule with an affinity for a SWNT of <1E−9 M.

A6.1.3.7: A cell-encoded peptide library, a medium comprising an organic solvent, a SWNT, and a library molecule with an affinity for a SWNT of <1E−10 M.

A6.1.3.8: A cell-encoded peptide library, a medium comprising an organic solvent, a SWNT, and a library molecule with an affinity for a SWNT of <1E−12 M.

A6.1.4.1: A cell-encoded peptide library, a medium comprising an organic solvent, a MWNT, and a library molecule with an affinity for a MWNT of <1E−2 M.

A6.1.4.2: A cell-encoded peptide library, a medium comprising an organic solvent, a MWNT, and a library molecule with an affinity for a MWNT of <1E−4 M.

A6.1.4.3: A cell-encoded peptide library, a medium comprising an organic solvent, a MWNT, and a library molecule with an affinity for a MWNT of <1E−6 M.

A6.1.4.4: A cell-encoded peptide library, a medium comprising an organic solvent, a MWNT, and a library molecule with an affinity for a MWNT of <1E−7 M.

A6.1.4.5: A cell-encoded peptide library, a medium comprising an organic solvent, a MWNT, and a library molecule with an affinity for a MWNT of <1E−8 M.

A6.1.4.6: A cell-encoded peptide library, a medium comprising an organic solvent, a MWNT, and a library molecule with an affinity for a MWNT of <1E−9 M.

A6.1.4.7: A cell-encoded peptide library, a medium comprising an organic solvent, a MWNT, and a library molecule with an affinity for a MWNT of <1E−10 M.

A6.1.4.8: A cell-encoded peptide library, a medium comprising an organic solvent, a MWNT, and a library molecule with an affinity for a MWNT of <1E−12 M.

A6.1.5.1: A cell-encoded peptide library, a medium comprising an organic solvent, a nanotube, and a library molecule with an affinity for a nanotube of <1E−2 M.

A6.1.5.2: A cell-encoded peptide library, a medium comprising an organic solvent, a nanotube, and a library molecule with an affinity for a nanotube of <1E−4 M.

A6.1.5.3: A cell-encoded peptide library, a medium comprising an organic solvent, a nanotube, and a library molecule with an affinity for a nanotube of <1E−6 M.

A6.1.5.4: A cell-encoded peptide library, a medium comprising an organic solvent, a nanotube, and a library molecule with an affinity for a nanotube of <1E−7 M.

A6.1.5.5: A cell-encoded peptide library, a medium comprising an organic solvent, a nanotube, and a library molecule with an affinity for a nanotube of <1E−8 M.

A6.1.5.6: A cell-encoded peptide library, a medium comprising an organic solvent, a nanotube, and a library molecule with an affinity for a nanotube of <1E−9 M.

A6.1.5.7: A cell-encoded peptide library, a medium comprising an organic solvent, a nanotube, and a library molecule with an affinity for a nanotube of <1E−10 M.

A6.1.5.8: A cell-encoded peptide library, a medium comprising an organic solvent, a nanotube, and a library molecule with an affinity for a nanotube of <1E−12 M.

A6.1.6.1: A cell-encoded peptide library, a medium comprising an organic solvent, a fullerene, and a library molecule with an affinity for a fullerene of <1E−2 M.

A6.1.6.2: A cell-encoded peptide library, a medium comprising an organic solvent, a fullerene, and a library molecule with an affinity for a fullerene of <1E−4 M.

A6.1.6.3: A cell-encoded peptide library, a medium comprising an organic solvent, a fullerene, and a library molecule with an affinity for a fullerene of <1E−6 M.

A6.1.6.4: A cell-encoded peptide library, a medium comprising an organic solvent, a fullerene, and a library molecule with an affinity for a fullerene of <1E−7 M.

A6.1.6.5: A cell-encoded peptide library, a medium comprising an organic solvent, a fullerene, and a library molecule with an affinity for a fullerene of <1E−8 M.

A6.1.6.6: A cell-encoded peptide library, a medium comprising an organic solvent, a fullerene, and a library molecule with an affinity for a fullerene of <1E−9 M.

A6.1.6.7: A cell-encoded peptide library, a medium comprising an organic solvent, a fullerene, and a library molecule with an affinity for a fullerene of <1E−10 M.

A6.1.6.8: A cell-encoded peptide library, a medium comprising an organic solvent, a fullerene, and a library molecule with an affinity for a fullerene of <1E−12 M.

A6.1.7.1: A cell-encoded peptide library, a medium comprising an organic solvent, a BNNT, and a library molecule with an affinity for a BNNT of <1E−2 M.

A6.1.7.2: A cell-encoded peptide library, a medium comprising an organic solvent, a BNNT, and a library molecule with an affinity for a BNNT of <1E−4 M.

A6.1.7.3: A cell-encoded peptide library, a medium comprising an organic solvent, a BNNT, and a library molecule with an affinity for a BNNT of <1E−6 M.

A6.1.7.4: A cell-encoded peptide library, a medium comprising an organic solvent, a BNNT, and a library molecule with an affinity for a BNNT of <1E−7 M.

A6.1.7.5: A cell-encoded peptide library, a medium comprising an organic solvent, a BNNT, and a library molecule with an affinity for a BNNT of <1E−8 M.

A6.1.7.6: A cell-encoded peptide library, a medium comprising an organic solvent, a BNNT, and a library molecule with an affinity for a BNNT of <1E−9 M.

A6.1.7.7: A cell-encoded peptide library, a medium comprising an organic solvent, a BNNT, and a library molecule with an affinity for a BNNT of <1E−10 M.

A6.1.7.8: A cell-encoded peptide library, a medium comprising an organic solvent, a BNNT, and a library molecule with an affinity for a BNNT of <1E−12 M.

A6.2.1.1: A cell-encoded peptide library, a medium comprising an organic solvent and a soluble polymer, a CNT, and a library molecule with an affinity for a CNT of <1E−2 M.

A6.2.1.2: A cell-encoded peptide library, a medium comprising an organic solvent and a soluble polymer, a CNT, and a library molecule with an affinity for a CNT of <1E−4 M.

A6.2.1.3: A cell-encoded peptide library, a medium comprising an organic solvent and a soluble polymer, a CNT, and a library molecule with an affinity for a CNT of <1E−6 M.

A6.2.1.4: A cell-encoded peptide library, a medium comprising an organic solvent and a soluble polymer, a CNT, and a library molecule with an affinity for a CNT of <1E−7 M.

A6.2.1.5: A cell-encoded peptide library, a medium comprising an organic solvent and a soluble polymer, a CNT, and a library molecule with an affinity for a CNT of <1E−8 M.

A6.2.1.6: A cell-encoded peptide library, a medium comprising an organic solvent and a soluble polymer, a CNT, and a library molecule with an affinity for a CNT of <1E−9 M.

A6.2.1.7: A cell-encoded peptide library, a medium comprising an organic solvent and a soluble polymer, a CNT, and a library molecule with an affinity for a CNT of <1E−10 M.

A6.2.1.8: A cell-encoded peptide library, a medium comprising an organic solvent and a soluble polymer, a CNT, and a library molecule with an affinity for a CNT of <1E−12 M.

A6.2.2.1: A cell-encoded peptide library, a medium comprising an organic solvent and a soluble polymer, a GS, and a library molecule with an affinity for a GS of <1E−2 M.

A6.2.2.2: A cell-encoded peptide library, a medium comprising an organic solvent and a soluble polymer, a GS, and a library molecule with an affinity for a GS of <1E−4 M.

A6.2.2.3: A cell-encoded peptide library, a medium comprising an organic solvent and a soluble polymer, a GS, and a library molecule with an affinity for a GS of <1E−6 M.

A6.2.2.4: A cell-encoded peptide library, a medium comprising an organic solvent and a soluble polymer, a GS, and a library molecule with an affinity for a GS of <1E−7 M.

A6.2.2.5: A cell-encoded peptide library, a medium comprising an organic solvent and a soluble polymer, a GS, and a library molecule with an affinity for a GS of <1E−8 M.

A6.2.2.6: A cell-encoded peptide library, a medium comprising an organic solvent and a soluble polymer, a GS, and a library molecule with an affinity for a GS of <1E−9 M.

A6.2.2.7: A cell-encoded peptide library, a medium comprising an organic solvent and a soluble polymer, a GS, and a library molecule with an affinity for a GS of <1E−10 M.

A6.2.2.8: A cell-encoded peptide library, a medium comprising an organic solvent and a soluble polymer, a GS, and a library molecule with an affinity for a GS of <1E−12 M.

A6.2.3.1: A cell-encoded peptide library, a medium comprising an organic solvent and a soluble polymer, a SWNT, and a library molecule with an affinity for a SWNT of <1E−2 M.

A6.2.3.2: A cell-encoded peptide library, a medium comprising an organic solvent and a soluble polymer, a SWNT, and a library molecule with an affinity for a SWNT of <1E−4 M.

A6.2.3.3: A cell-encoded peptide library, a medium comprising an organic solvent and a soluble polymer, a SWNT, and a library molecule with an affinity for a SWNT of <1E−6 M.

A6.2.3.4: A cell-encoded peptide library, a medium comprising an organic solvent and a soluble polymer, a SWNT, and a library molecule with an affinity for a SWNT of <1E−7 M.

A6.2.3.5: A cell-encoded peptide library, a medium comprising an organic solvent and a soluble polymer, a SWNT, and a library molecule with an affinity for a SWNT of <1E−8 M.

A6.2.3.6: A cell-encoded peptide library, a medium comprising an organic solvent and a soluble polymer, a SWNT, and a library molecule with an affinity for a SWNT of <1E−9 M.

A6.2.3.7: A cell-encoded peptide library, a medium comprising an organic solvent and a soluble polymer, a SWNT, and a library molecule with an affinity for a SWNT of <1E−10 M.

A6.2.3.8: A cell-encoded peptide library, a medium comprising an organic solvent and a soluble polymer, a SWNT, and a library molecule with an affinity for a SWNT of <1E−12 M.

A6.2.4.1: A cell-encoded peptide library, a medium comprising an organic solvent and a soluble polymer, a MWNT, and a library molecule with an affinity for a MWNT of <1E−2 M.

A6.2.4.2: A cell-encoded peptide library, a medium comprising an organic solvent and a soluble polymer, a MWNT, and a library molecule with an affinity for a MWNT of <1E−4 M.

A6.2.4.3: A cell-encoded peptide library, a medium comprising an organic solvent and a soluble polymer, a MWNT, and a library molecule with an affinity for a MWNT of <1E−6 M.

A6.2.4.4: A cell-encoded peptide library, a medium comprising an organic solvent and a soluble polymer, a MWNT, and a library molecule with an affinity for a MWNT of <1E−7 M.

A6.2.4.5: A cell-encoded peptide library, a medium comprising an organic solvent and a soluble polymer, a MWNT, and a library molecule with an affinity for a MWNT of <1E−8 M.

A6.2.4.6: A cell-encoded peptide library, a medium comprising an organic solvent and a soluble polymer, a MWNT, and a library molecule with an affinity for a MWNT of <1E−9 M.

A6.2.4.7: A cell-encoded peptide library, a medium comprising an organic solvent and a soluble polymer, a MWNT, and a library molecule with an affinity for a MWNT of <1E−10 M.

A6.2.4.8: A cell-encoded peptide library, a medium comprising an organic solvent and a soluble polymer, a MWNT, and a library molecule with an affinity for a MWNT of <1E−12 M.

A6.2.5.1: A cell-encoded peptide library, a medium comprising an organic solvent and a soluble polymer, a nanotube, and a library molecule with an affinity for a nanotube of <1E−2 M.

A6.2.5.2: A cell-encoded peptide library, a medium comprising an organic solvent and a soluble polymer, a nanotube, and a library molecule with an affinity for a nanotube of <1E−4 M.

A6.2.5.3: A cell-encoded peptide library, a medium comprising an organic solvent and a soluble polymer, a nanotube, and a library molecule with an affinity for a nanotube of <1E−6 M.

A6.2.5.4: A cell-encoded peptide library, a medium comprising an organic solvent and a soluble polymer, a nanotube, and a library molecule with an affinity for a nanotube of <1E−7 M.

A6.2.5.5: A cell-encoded peptide library, a medium comprising an organic solvent and a soluble polymer, a nanotube, and a library molecule with an affinity for a nanotube of <1E−8 M.

A6.2.5.6: A cell-encoded peptide library, a medium comprising an organic solvent and a soluble polymer, a nanotube, and a library molecule with an affinity for a nanotube of <1E−9 M.

A6.2.5.7: A cell-encoded peptide library, a medium comprising an organic solvent and a soluble polymer, a nanotube, and a library molecule with an affinity for a nanotube of <1E−10 M.

A6.2.5.8: A cell-encoded peptide library, a medium comprising an organic solvent and a soluble polymer, a nanotube, and a library molecule with an affinity for a nanotube of <1E−12 M.

A6.2.6.1: A cell-encoded peptide library, a medium comprising an organic solvent and a soluble polymer, a fullerene, and a library molecule with an affinity for a fullerene of <1E−2 M.

A6.2.6.2: A cell-encoded peptide library, a medium comprising an organic solvent and a soluble polymer, a fullerene, and a library molecule with an affinity for a fullerene of <1E−4 M.

A6.2.6.3: A cell-encoded peptide library, a medium comprising an organic solvent and a soluble polymer, a fullerene, and a library molecule with an affinity for a fullerene of <1E−6 M.

A6.2.6.4: A cell-encoded peptide library, a medium comprising an organic solvent and a soluble polymer, a fullerene, and a library molecule with an affinity for a fullerene of <1E−7 M.

A6.2.6.5: A cell-encoded peptide library, a medium comprising an organic solvent and a soluble polymer, a fullerene, and a library molecule with an affinity for a fullerene of <1E−8 M.

A6.2.6.6: A cell-encoded peptide library, a medium comprising an organic solvent and a soluble polymer, a fullerene, and a library molecule with an affinity for a fullerene of <1E−9 M.

A6.2.6.7: A cell-encoded peptide library, a medium comprising an organic solvent and a soluble polymer, a fullerene, and a library molecule with an affinity for a fullerene of <1E−10 M.

A6.2.6.8: A cell-encoded peptide library, a medium comprising an organic solvent and a soluble polymer, a fullerene, and a library molecule with an affinity for a fullerene of <1E−12 M.

A6.2.7.1: A cell-encoded peptide library, a medium comprising an organic solvent and a soluble polymer, a BNNT, and a library molecule with an affinity for a BNNT of <1E−2 M.

A6.2.7.2: A cell-encoded peptide library, a medium comprising an organic solvent and a soluble polymer, a BNNT, and a library molecule with an affinity for a BNNT of <1E−4 M.

A6.2.7.3: A cell-encoded peptide library, a medium comprising an organic solvent and a soluble polymer, a BNNT, and a library molecule with an affinity for a BNNT of <1E−6 M.

A6.2.7.4: A cell-encoded peptide library, a medium comprising an organic solvent and a soluble polymer, a BNNT, and a library molecule with an affinity for a BNNT of <1E−7 M.

A6.2.7.5: A cell-encoded peptide library, a medium comprising an organic solvent and a soluble polymer, a BNNT, and a library molecule with an affinity for a BNNT of <1E−8 M.

A6.2.7.6: A cell-encoded peptide library, a medium comprising an organic solvent and a soluble polymer, a BNNT, and a library molecule with an affinity for a BNNT of <1E−9 M.

A6.2.7.7: A cell-encoded peptide library, a medium comprising an organic solvent and a soluble polymer, a BNNT, and a library molecule with an affinity for a BNNT of <1E−10 M.

A6.2.7.8: A cell-encoded peptide library, a medium comprising an organic solvent and a soluble polymer, a BNNT, and a library molecule with an affinity for a BNNT of <1E−12 M.

A6.3.1.1: A cell-encoded peptide library, a medium comprising a soluble polymer, a CNT, and a library molecule with an affinity for a CNT of <1E−2 M.

A6.3.1.2: A cell-encoded peptide library, a medium comprising a soluble polymer, a CNT, and a library molecule with an affinity for a CNT of <1E−4 M.

A6.3.1.3: A cell-encoded peptide library, a medium comprising a soluble polymer, a CNT, and a library molecule with an affinity for a CNT of <1E−6 M.

A6.3.1.4: A cell-encoded peptide library, a medium comprising a soluble polymer, a CNT, and a library molecule with an affinity for a CNT of <1E−7 M.

A6.3.1.5: A cell-encoded peptide library, a medium comprising a soluble polymer, a CNT, and a library molecule with an affinity for a CNT of <1E−8 M.

A6.3.1.6: A cell-encoded peptide library, a medium comprising a soluble polymer, a CNT, and a library molecule with an affinity for a CNT of <1E−9 M.

A6.3.1.7: A cell-encoded peptide library, a medium comprising a soluble polymer, a CNT, and a library molecule with an affinity for a CNT of <1E−10 M.

A6.3.1.8: A cell-encoded peptide library, a medium comprising a soluble polymer, a CNT, and a library molecule with an affinity for a CNT of <1E−12 M.

A6.3.2.1: A cell-encoded peptide library, a medium comprising a soluble polymer, a GS, and a library molecule with an affinity for a GS of <1E−2 M.

A6.3.2.2: A cell-encoded peptide library, a medium comprising a soluble polymer, a GS, and a library molecule with an affinity for a GS of <1E−4 M.

A6.3.2.3: A cell-encoded peptide library, a medium comprising a soluble polymer, a GS, and a library molecule with an affinity for a GS of <1E−6 M.

A6.3.2.4: A cell-encoded peptide library, a medium comprising a soluble polymer, a GS, and a library molecule with an affinity for a GS of <1E−7 M.

A6.3.2.5: A cell-encoded peptide library, a medium comprising a soluble polymer, a GS, and a library molecule with an affinity for a GS of <1E−8 M.

A6.3.2.6: A cell-encoded peptide library, a medium comprising a soluble polymer, a GS, and a library molecule with an affinity for a GS of <1E−9 M.

A6.3.2.7: A cell-encoded peptide library, a medium comprising a soluble polymer, a GS, and a library molecule with an affinity for a GS of <1E−10 M.

A6.3.2.8: A cell-encoded peptide library, a medium comprising a soluble polymer, a GS, and a library molecule with an affinity for a GS of <1E−12 M.

A6.3.3.1: A cell-encoded peptide library, a medium comprising a soluble polymer, a SWNT, and a library molecule with an affinity for a SWNT of <1E−2 M.

A6.3.3.2: A cell-encoded peptide library, a medium comprising a soluble polymer, a SWNT, and a library molecule with an affinity for a SWNT of <1E−4 M.

A6.3.3.3: A cell-encoded peptide library, a medium comprising a soluble polymer, a SWNT, and a library molecule with an affinity for a SWNT of <1E−6 M.

A6.3.3.4: A cell-encoded peptide library, a medium comprising a soluble polymer, a SWNT, and a library molecule with an affinity for a SWNT of <1E−7 M.

A6.3.3.5: A cell-encoded peptide library, a medium comprising a soluble polymer, a SWNT, and a library molecule with an affinity for a SWNT of <1E−8 M.

A6.3.3.6: A cell-encoded peptide library, a medium comprising a soluble polymer, a SWNT, and a library molecule with an affinity for a SWNT of <1E−9 M.

A6.3.3.7: A cell-encoded peptide library, a medium comprising a soluble polymer, a SWNT, and a library molecule with an affinity for a SWNT of <1E−10 M.

A6.3.3.8: A cell-encoded peptide library, a medium comprising a soluble polymer, a SWNT, and a library molecule with an affinity for a SWNT of <1E−12 M.

A6.3.4.1: A cell-encoded peptide library, a medium comprising a soluble polymer, a MWNT, and a library molecule with an affinity for a MWNT of <1E−2 M.

A6.3.4.2: A cell-encoded peptide library, a medium comprising a soluble polymer, a MWNT, and a library molecule with an affinity for a MWNT of <1E−4 M.

A6.3.4.3: A cell-encoded peptide library, a medium comprising a soluble polymer, a MWNT, and a library molecule with an affinity for a MWNT of <1E−6 M.

A6.3.4.4: A cell-encoded peptide library, a medium comprising a soluble polymer, a MWNT, and a library molecule with an affinity for a MWNT of <1E−7 M.

A6.3.4.5: A cell-encoded peptide library, a medium comprising a soluble polymer, a MWNT, and a library molecule with an affinity for a MWNT of <1E−8 M.

A6.3.4.6: A cell-encoded peptide library, a medium comprising a soluble polymer, a MWNT, and a library molecule with an affinity for a MWNT of <1E−9 M.

A6.3.4.7: A cell-encoded peptide library, a medium comprising a soluble polymer, a MWNT, and a library molecule with an affinity for a MWNT of <1E−10 M.

A6.3.4.8: A cell-encoded peptide library, a medium comprising a soluble polymer, a MWNT, and a library molecule with an affinity for a MWNT of <1E−12 M.

A6.3.5.1: A cell-encoded peptide library, a medium comprising a soluble polymer, a nanotube, and a library molecule with an affinity for a nanotube of <1E−2 M.

A6.3.5.2: A cell-encoded peptide library, a medium comprising a soluble polymer, a nanotube, and a library molecule with an affinity for a nanotube of <1E−4 M.

A6.3.5.3: A cell-encoded peptide library, a medium comprising a soluble polymer, a nanotube, and a library molecule with an affinity for a nanotube of <1E−6 M.

A6.3.5.4: A cell-encoded peptide library, a medium comprising a soluble polymer, a nanotube, and a library molecule with an affinity for a nanotube of <1E−7 M.

A6.3.5.5: A cell-encoded peptide library, a medium comprising a soluble polymer, a nanotube, and a library molecule with an affinity for a nanotube of <1E−8 M.

A6.3.5.6: A cell-encoded peptide library, a medium comprising a soluble polymer, a nanotube, and a library molecule with an affinity for a nanotube of <1E−9 M.

A6.3.5.7: A cell-encoded peptide library, a medium comprising a soluble polymer, a nanotube, and a library molecule with an affinity for a nanotube of <1E−10 M.

A6.3.5.8: A cell-encoded peptide library, a medium comprising a soluble polymer, a nanotube, and a library molecule with an affinity for a nanotube of <1E−12 M.

A6.3.6.1: A cell-encoded peptide library, a medium comprising a soluble polymer, a fullerene, and a library molecule with an affinity for a fullerene of <1E−2 M.

A6.3.6.2: A cell-encoded peptide library, a medium comprising a soluble polymer, a fullerene, and a library molecule with an affinity for a fullerene of <1E−4 M.

A6.3.6.3: A cell-encoded peptide library, a medium comprising a soluble polymer, a fullerene, and a library molecule with an affinity for a fullerene of <1E−6 M.

A6.3.6.4: A cell-encoded peptide library, a medium comprising a soluble polymer, a fullerene, and a library molecule with an affinity for a fullerene of <1E−7 M.

A6.3.6.5: A cell-encoded peptide library, a medium comprising a soluble polymer, a fullerene, and a library molecule with an affinity for a fullerene of <1E−8 M.

A6.3.6.6: A cell-encoded peptide library, a medium comprising a soluble polymer, a fullerene, and a library molecule with an affinity for a fullerene of <1E−9 M.

A6.3.6.7: A cell-encoded peptide library, a medium comprising a soluble polymer, a fullerene, and a library molecule with an affinity for a fullerene of <1E−10 M.

A6.3.6.8: A cell-encoded peptide library, a medium comprising a soluble polymer, a fullerene, and a library molecule with an affinity for a fullerene of <1E−12 M.

A6.3.7.1: A cell-encoded peptide library, a medium comprising a soluble polymer, a BNNT, and a library molecule with an affinity for a BNNT of <1E−2 M.

A6.3.7.2: A cell-encoded peptide library, a medium comprising a soluble polymer, a BNNT, and a library molecule with an affinity for a BNNT of <1E−4 M.

A6.3.7.3: A cell-encoded peptide library, a medium comprising a soluble polymer, a BNNT, and a library molecule with an affinity for a BNNT of <1E−6 M.

A6.3.7.4: A cell-encoded peptide library, a medium comprising a soluble polymer, a BNNT, and a library molecule with an affinity for a BNNT of <1E−7 M.

A6.3.7.5: A cell-encoded peptide library, a medium comprising a soluble polymer, a BNNT, and a library molecule with an affinity for a BNNT of <1E−8 M.

A6.3.7.6: A cell-encoded peptide library, a medium comprising a soluble polymer, a BNNT, and a library molecule with an affinity for a BNNT of <1E−9 M.

A6.3.7.7: A cell-encoded peptide library, a medium comprising a soluble polymer, a BNNT, and a library molecule with an affinity for a BNNT of <1E−10 M.

A6.3.7.8: A cell-encoded peptide library, a medium comprising a soluble polymer, a BNNT, and a library molecule with an affinity for a BNNT of <1E−12 M.

A6.4.1.1: A cell-encoded peptide library, a medium comprising >50% organic solvent, a CNT, and a library molecule with an affinity for a CNT of <1E−2 M.

A6.4.1.2: A cell-encoded peptide library, a medium comprising >50% organic solvent, a CNT, and a library molecule with an affinity for a CNT of <1E−4 M.

A6.4.1.3: A cell-encoded peptide library, a medium comprising >50% organic solvent, a CNT, and a library molecule with an affinity for a CNT of <1E−6 M.

A6.4.1.4: A cell-encoded peptide library, a medium comprising >50% organic solvent, a CNT, and a library molecule with an affinity for a CNT of <1E−7 M.

A6.4.1.5: A cell-encoded peptide library, a medium comprising >50% organic solvent, a CNT, and a library molecule with an affinity for a CNT of <1E−8 M.

A6.4.1.6: A cell-encoded peptide library, a medium comprising >50% organic solvent, a CNT, and a library molecule with an affinity for a CNT of <1E−9 M.

A6.4.1.7: A cell-encoded peptide library, a medium comprising >50% organic solvent, a CNT, and a library molecule with an affinity for a CNT of <1E−10 M.

A6.4.1.8: A cell-encoded peptide library, a medium comprising >50% organic solvent, a CNT, and a library molecule with an affinity for a CNT of <1E−12 M.

A6.4.2.1: A cell-encoded peptide library, a medium comprising >50% organic solvent, a GS, and a library molecule with an affinity for a GS of <1E−2 M.

A6.4.2.2: A cell-encoded peptide library, a medium comprising >50% organic solvent, a GS, and a library molecule with an affinity for a GS of <1E−4 M.

A6.4.2.3: A cell-encoded peptide library, a medium comprising >50% organic solvent, a GS, and a library molecule with an affinity for a GS of <1E−6 M.

A6.4.2.4: A cell-encoded peptide library, a medium comprising >50% organic solvent, a GS, and a library molecule with an affinity for a GS of <1E−7 M.

A6.4.2.5: A cell-encoded peptide library, a medium comprising >50% organic solvent, a GS, and a library molecule with an affinity for a GS of <1E−8 M.

A6.4.2.6: A cell-encoded peptide library, a medium comprising >50% organic solvent, a GS, and a library molecule with an affinity for a GS of <1E−9 M.

A6.4.2.7: A cell-encoded peptide library, a medium comprising >50% organic solvent, a GS, and a library molecule with an affinity for a GS of <1E−10 M.

A6.4.2.8: A cell-encoded peptide library, a medium comprising >50% organic solvent, a GS, and a library molecule with an affinity for a GS of <1E−12 M.

A6.4.3.1: A cell-encoded peptide library, a medium comprising >50% organic solvent, a SWNT, and a library molecule with an affinity for a SWNT of <1E−2 M.

A6.4.3.2: A cell-encoded peptide library, a medium comprising >50% organic solvent, a SWNT, and a library molecule with an affinity for a SWNT of <1E−4 M.

A6.4.3.3: A cell-encoded peptide library, a medium comprising >50% organic solvent, a SWNT, and a library molecule with an affinity for a SWNT of <1E−6 M.

A6.4.3.4: A cell-encoded peptide library, a medium comprising >50% organic solvent, a SWNT, and a library molecule with an affinity for a SWNT of <1E−7 M.

A6.4.3.5: A cell-encoded peptide library, a medium comprising >50% organic solvent, a SWNT, and a library molecule with an affinity for a SWNT of <1E−8 M.

A6.4.3.6: A cell-encoded peptide library, a medium comprising >50% organic solvent, a SWNT, and a library molecule with an affinity for a SWNT of <1E−9 M.

A6.4.3.7: A cell-encoded peptide library, a medium comprising >50% organic solvent, a SWNT, and a library molecule with an affinity for a SWNT of <1E−10 M.

A6.4.3.8: A cell-encoded peptide library, a medium comprising >50% organic solvent, a SWNT, and a library molecule with an affinity for a SWNT of <1E−12 M.

A6.4.4.1: A cell-encoded peptide library, a medium comprising >50% organic solvent, a MWNT, and a library molecule with an affinity for a MWNT of <1E−2 M.

A6.4.4.2: A cell-encoded peptide library, a medium comprising >50% organic solvent, a MWNT, and a library molecule with an affinity for a MWNT of <1E−4 M.

A6.4.4.3: A cell-encoded peptide library, a medium comprising >50% organic solvent, a MWNT, and a library molecule with an affinity for a MWNT of <1E−6 M.

A6.4.4.4: A cell-encoded peptide library, a medium comprising >50% organic solvent, a MWNT, and a library molecule with an affinity for a MWNT of <1E−7 M.

A6.4.4.5: A cell-encoded peptide library, a medium comprising >50% organic solvent, a MWNT, and a library molecule with an affinity for a MWNT of <1E−8 M.

A6.4.4.6: A cell-encoded peptide library, a medium comprising >50% organic solvent, a MWNT, and a library molecule with an affinity for a MWNT of <1E−9 M.

A6.4.4.7: A cell-encoded peptide library, a medium comprising >50% organic solvent, a MWNT, and a library molecule with an affinity for a MWNT of <1E−10 M.

A6.4.4.8: A cell-encoded peptide library, a medium comprising >50% organic solvent, a MWNT, and a library molecule with an affinity for a MWNT of <1E−12 M.

A6.4.5.1: A cell-encoded peptide library, a medium comprising >50% organic solvent, a nanotube, and a library molecule with an affinity for a nanotube of <1E−2 M.

A6.4.5.2: A cell-encoded peptide library, a medium comprising >50% organic solvent, a nanotube, and a library molecule with an affinity for a nanotube of <1E−4 M.

A6.4.5.3: A cell-encoded peptide library, a medium comprising >50% organic solvent, a nanotube, and a library molecule with an affinity for a nanotube of <1E−6 M.

A6.4.5.4: A cell-encoded peptide library, a medium comprising >50% organic solvent, a nanotube, and a library molecule with an affinity for a nanotube of <1E−7 M.

A6.4.5.5: A cell-encoded peptide library, a medium comprising >50% organic solvent, a nanotube, and a library molecule with an affinity for a nanotube of <1E−8 M.

A6.4.5.6: A cell-encoded peptide library, a medium comprising >50% organic solvent, a nanotube, and a library molecule with an affinity for a nanotube of <1E−9 M.

A6.4.5.7: A cell-encoded peptide library, a medium comprising >50% organic solvent, a nanotube, and a library molecule with an affinity for a nanotube of <1E−10 M.

A6.4.5.8: A cell-encoded peptide library, a medium comprising >50% organic solvent, a nanotube, and a library molecule with an affinity for a nanotube of <1E−12 M.

A6.4.6.1: A cell-encoded peptide library, a medium comprising >50% organic solvent, a fullerene, and a library molecule with an affinity for a fullerene of <1E−2 M.

A6.4.6.2: A cell-encoded peptide library, a medium comprising >50% organic solvent, a fullerene, and a library molecule with an affinity for a fullerene of <1E−4 M.

A6.4.6.3: A cell-encoded peptide library, a medium comprising >50% organic solvent, a fullerene, and a library molecule with an affinity for a fullerene of <1E−6 M.

A6.4.6.4: A cell-encoded peptide library, a medium comprising >50% organic solvent, a fullerene, and a library molecule with an affinity for a fullerene of <1E−7 M.

A6.4.6.5: A cell-encoded peptide library, a medium comprising >50% organic solvent, a fullerene, and a library molecule with an affinity for a fullerene of <1E−8 M.

A6.4.6.6: A cell-encoded peptide library, a medium comprising >50% organic solvent, a fullerene, and a library molecule with an affinity for a fullerene of <1E−9 M.

A6.4.6.7: A cell-encoded peptide library, a medium comprising >50% organic solvent, a fullerene, and a library molecule with an affinity for a fullerene of <1E−10 M.

A6.4.6.8: A cell-encoded peptide library, a medium comprising >50% organic solvent, a fullerene, and a library molecule with an affinity for a fullerene of <1E−12 M.

A6.4.7.1: A cell-encoded peptide library, a medium comprising >50% organic solvent, a BNNT, and a library molecule with an affinity for a BNNT of <1E−2 M.

A6.4.7.2: A cell-encoded peptide library, a medium comprising >50% organic solvent, a BNNT, and a library molecule with an affinity for a BNNT of <1E−4 M.

A6.4.7.3: A cell-encoded peptide library, a medium comprising >50% organic solvent, a BNNT, and a library molecule with an affinity for a BNNT of <1E−6 M.

A6.4.7.4: A cell-encoded peptide library, a medium comprising >50% organic solvent, a BNNT, and a library molecule with an affinity for a BNNT of <1E−7 M.

A6.4.7.5: A cell-encoded peptide library, a medium comprising >50% organic solvent, a BNNT, and a library molecule with an affinity for a BNNT of <1E−8 M.

A6.4.7.6: A cell-encoded peptide library, a medium comprising >50% organic solvent, a BNNT, and a library molecule with an affinity for a BNNT of <1E−9 M.

A6.4.7.7: A cell-encoded peptide library, a medium comprising >50% organic solvent, a BNNT, and a library molecule with an affinity for a BNNT of <1E−10 M.

A6.4.7.8: A cell-encoded peptide library, a medium comprising >50% organic solvent, a BNNT, and a library molecule with an affinity for a BNNT of <1E−12 M.

A6.5.1.1: A cell-encoded peptide library, a medium comprising >50% organic solvent and a soluble polymer, a CNT, and a library molecule with an affinity for a CNT of <1E−2 M.

A6.5.1.2: A cell-encoded peptide library, a medium comprising >50% organic solvent and a soluble polymer, a CNT, and a library molecule with an affinity for a CNT of <1E−4 M.

A6.5.1.3: A cell-encoded peptide library, a medium comprising >50% organic solvent and a soluble polymer, a CNT, and a library molecule with an affinity for a CNT of <1E−6 M.

A6.5.1.4: A cell-encoded peptide library, a medium comprising >50% organic solvent and a soluble polymer, a CNT, and a library molecule with an affinity for a CNT of <1E−7 M.

A6.5.1.5: A cell-encoded peptide library, a medium comprising >50% organic solvent and a soluble polymer, a CNT, and a library molecule with an affinity for a CNT of <1E−8 M.

A6.5.1.6: A cell-encoded peptide library, a medium comprising >50% organic solvent and a soluble polymer, a CNT, and a library molecule with an affinity for a CNT of <1E−9 M.

A6.5.1.7: A cell-encoded peptide library, a medium comprising >50% organic solvent and a soluble polymer, a CNT, and a library molecule with an affinity for a CNT of <1E−10 M.

A6.5.1.8: A cell-encoded peptide library, a medium comprising >50% organic solvent and a soluble polymer, a CNT, and a library molecule with an affinity for a CNT of <1E−12 M.

A6.5.2.1: A cell-encoded peptide library, a medium comprising >50% organic solvent and a soluble polymer, a GS, and a library molecule with an affinity for a GS of <1E−2 M.

A6.5.2.2: A cell-encoded peptide library, a medium comprising >50% organic solvent and a soluble polymer, a GS, and a library molecule with an affinity for a GS of <1E−4 M.

A6.5.2.3: A cell-encoded peptide library, a medium comprising >50% organic solvent and a soluble polymer, a GS, and a library molecule with an affinity for a GS of <1E−6 M.

A6.5.2.4: A cell-encoded peptide library, a medium comprising >50% organic solvent and a soluble polymer, a GS, and a library molecule with an affinity for a GS of <1E−7 M.

A6.5.2.5: A cell-encoded peptide library, a medium comprising >50% organic solvent and a soluble polymer, a GS, and a library molecule with an affinity for a GS of <1E−8 M.

A6.5.2.6: A cell-encoded peptide library, a medium comprising >50% organic solvent and a soluble polymer, a GS, and a library molecule with an affinity for a GS of <1E−9 M.

A6.5.2.7: A cell-encoded peptide library, a medium comprising >50% organic solvent and a soluble polymer, a GS, and a library molecule with an affinity for a GS of <1E−10 M.

A6.5.2.8: A cell-encoded peptide library, a medium comprising >50% organic solvent and a soluble polymer, a GS, and a library molecule with an affinity for a GS of <1E−12 M.

A6.5.3.1: A cell-encoded peptide library, a medium comprising >50% organic solvent and a soluble polymer, a SWNT, and a library molecule with an affinity for a SWNT of <1E−2 M.

A6.5.3.2: A cell-encoded peptide library, a medium comprising >50% organic solvent and a soluble polymer, a SWNT, and a library molecule with an affinity for a SWNT of <1E−4 M.

A6.5.3.3: A cell-encoded peptide library, a medium comprising >50% organic solvent and a soluble polymer, a SWNT, and a library molecule with an affinity for a SWNT of <1E−6 M.

A6.5.3.4: A cell-encoded peptide library, a medium comprising >50% organic solvent and a soluble polymer, a SWNT, and a library molecule with an affinity for a SWNT of <1E−7 M.

A6.5.3.5: A cell-encoded peptide library, a medium comprising >50% organic solvent and a soluble polymer, a SWNT, and a library molecule with an affinity for a SWNT of <1E−8 M.

A6.5.3.6: A cell-encoded peptide library, a medium comprising >50% organic solvent and a soluble polymer, a SWNT, and a library molecule with an affinity for a SWNT of <1E−9 M.

A6.5.3.7: A cell-encoded peptide library, a medium comprising >50% organic solvent and a soluble polymer, a SWNT, and a library molecule with an affinity for a SWNT of <1E−10 M.

A6.5.3.8: A cell-encoded peptide library, a medium comprising >50% organic solvent and a soluble polymer, a SWNT, and a library molecule with an affinity for a SWNT of <1E−12 M.

A6.5.4.1: A cell-encoded peptide library, a medium comprising >50% organic solvent and a soluble polymer, a MWNT, and a library molecule with an affinity for a MWNT of <1E−2 M.

A6.5.4.2: A cell-encoded peptide library, a medium comprising >50% organic solvent and a soluble polymer, a MWNT, and a library molecule with an affinity for a MWNT of <1E−4 M.

A6.5.4.3: A cell-encoded peptide library, a medium comprising >50% organic solvent and a soluble polymer, a MWNT, and a library molecule with an affinity for a MWNT of <1E−6 M.

A6.5.4.4: A cell-encoded peptide library, a medium comprising >50% organic solvent and a soluble polymer, a MWNT, and a library molecule with an affinity for a MWNT of <1E−7 M.

A6.5.4.5: A cell-encoded peptide library, a medium comprising >50% organic solvent and a soluble polymer, a MWNT, and a library molecule with an affinity for a MWNT of <1E−8 M.

A6.5.4.6: A cell-encoded peptide library, a medium comprising >50% organic solvent and a soluble polymer, a MWNT, and a library molecule with an affinity for a MWNT of <1E−9 M.

A6.5.4.7: A cell-encoded peptide library, a medium comprising >50% organic solvent and a soluble polymer, a MWNT, and a library molecule with an affinity for a MWNT of <1E−10 M.

A6.5.4.8: A cell-encoded peptide library, a medium comprising >50% organic solvent and a soluble polymer, a MWNT, and a library molecule with an affinity for a MWNT of <1E−12 M.

A6.5.5.1: A cell-encoded peptide library, a medium comprising >50% organic solvent and a soluble polymer, a nanotube, and a library molecule with an affinity for a nanotube of <1E−2 M.

A6.5.5.2: A cell-encoded peptide library, a medium comprising >50% organic solvent and a soluble polymer, a nanotube, and a library molecule with an affinity for a nanotube of <1E−4 M.

A6.5.5.3: A cell-encoded peptide library, a medium comprising >50% organic solvent and a soluble polymer, a nanotube, and a library molecule with an affinity for a nanotube of <1E−6 M.

A6.5.5.4: A cell-encoded peptide library, a medium comprising >50% organic solvent and a soluble polymer, a nanotube, and a library molecule with an affinity for a nanotube of <1E−7 M.

A6.5.5.5: A cell-encoded peptide library, a medium comprising >50% organic solvent and a soluble polymer, a nanotube, and a library molecule with an affinity for a nanotube of <1E−8 M.

A6.5.5.6: A cell-encoded peptide library, a medium comprising >50% organic solvent and a soluble polymer, a nanotube, and a library molecule with an affinity for a nanotube of <1E−9 M.

A6.5.5.7: A cell-encoded peptide library, a medium comprising >50% organic solvent and a soluble polymer, a nanotube, and a library molecule with an affinity for a nanotube of <1E−10 M.

A6.5.5.8: A cell-encoded peptide library, a medium comprising >50% organic solvent and a soluble polymer, a nanotube, and a library molecule with an affinity for a nanotube of <1E−12 M.

A6.5.6.1: A cell-encoded peptide library, a medium comprising >50% organic solvent and a soluble polymer, a fullerene, and a library molecule with an affinity for a fullerene of <1E−2 M.

A6.5.6.2: A cell-encoded peptide library, a medium comprising >50% organic solvent and a soluble polymer, a fullerene, and a library molecule with an affinity for a fullerene of <1E−4 M.

A6.5.6.3: A cell-encoded peptide library, a medium comprising >50% organic solvent and a soluble polymer, a fullerene, and a library molecule with an affinity for a fullerene of <1E−6 M.

A6.5.6.4: A cell-encoded peptide library, a medium comprising >50% organic solvent and a soluble polymer, a fullerene, and a library molecule with an affinity for a fullerene of <1E−7 M.

A6.5.6.5: A cell-encoded peptide library, a medium comprising >50% organic solvent and a soluble polymer, a fullerene, and a library molecule with an affinity for a fullerene of <1E−8 M.

A6.5.6.6: A cell-encoded peptide library, a medium comprising >50% organic solvent and a soluble polymer, a fullerene, and a library molecule with an affinity for a fullerene of <1E−9 M.

A6.5.6.7: A cell-encoded peptide library, a medium comprising >50% organic solvent and a soluble polymer, a fullerene, and a library molecule with an affinity for a fullerene of <1E−10 M.

A6.5.6.8: A cell-encoded peptide library, a medium comprising >50% organic solvent and a soluble polymer, a fullerene, and a library molecule with an affinity for a fullerene of <1E−12 M.

A6.5.7.1: A cell-encoded peptide library, a medium comprising >50% organic solvent and a soluble polymer, a BNNT, and a library molecule with an affinity for a BNNT of <1E−2 M.

A6.5.7.2: A cell-encoded peptide library, a medium comprising >50% organic solvent and a soluble polymer, a BNNT, and a library molecule with an affinity for a BNNT of <1E−4 M.

A6.5.7.3: A cell-encoded peptide library, a medium comprising >50% organic solvent and a soluble polymer, a BNNT, and a library molecule with an affinity for a BNNT of <1E−6 M.

A6.5.7.4: A cell-encoded peptide library, a medium comprising >50% organic solvent and a soluble polymer, a BNNT, and a library molecule with an affinity for a BNNT of <1E−7 M.

A6.5.7.5: A cell-encoded peptide library, a medium comprising >50% organic solvent and a soluble polymer, a BNNT, and a library molecule with an affinity for a BNNT of <1E−8 M.

A6.5.7.6: A cell-encoded peptide library, a medium comprising >50% organic solvent and a soluble polymer, a BNNT, and a library molecule with an affinity for a BNNT of <1E−9 M.

A6.5.7.7: A cell-encoded peptide library, a medium comprising >50% organic solvent and a soluble polymer, a BNNT, and a library molecule with an affinity for a BNNT of <1E−10 M.

A6.5.7.8: A cell-encoded peptide library, a medium comprising >50% organic solvent and a soluble polymer, a BNNT, and a library molecule with an affinity for a BNNT of <1E−12 M.

A6.6.1.1: A cell-encoded peptide library, a medium comprising >80% organic solvent, a CNT, and a library molecule with an affinity for a CNT of <1E−2 M.

A6.6.1.2: A cell-encoded peptide library, a medium comprising >80% organic solvent, a CNT, and a library molecule with an affinity for a CNT of <1E−4 M.

A6.6.1.3: A cell-encoded peptide library, a medium comprising >80% organic solvent, a CNT, and a library molecule with an affinity for a CNT of <1E−6 M.

A6.6.1.4: A cell-encoded peptide library, a medium comprising >80% organic solvent, a CNT, and a library molecule with an affinity for a CNT of <1E−7 M.

A6.6.1.5: A cell-encoded peptide library, a medium comprising >80% organic solvent, a CNT, and a library molecule with an affinity for a CNT of <1E−8 M.

A6.6.1.6: A cell-encoded peptide library, a medium comprising >80% organic solvent, a CNT, and a library molecule with an affinity for a CNT of <1E−9 M.

A6.6.1.7: A cell-encoded peptide library, a medium comprising >80% organic solvent, a CNT, and a library molecule with an affinity for a CNT of <1E−10 M.

A6.6.1.8: A cell-encoded peptide library, a medium comprising >80% organic solvent, a CNT, and a library molecule with an affinity for a CNT of <1E−12 M.

A6.6.2.1: A cell-encoded peptide library, a medium comprising >80% organic solvent, a GS, and a library molecule with an affinity for a GS of <1E−2 M.

A6.6.2.2: A cell-encoded peptide library, a medium comprising >80% organic solvent, a GS, and a library molecule with an affinity for a GS of <1E−4 M.

A6.6.2.3: A cell-encoded peptide library, a medium comprising >80% organic solvent, a GS, and a library molecule with an affinity for a GS of <1E−6 M.

A6.6.2.4: A cell-encoded peptide library, a medium comprising >80% organic solvent, a GS, and a library molecule with an affinity for a GS of <1E−7 M.

A6.6.2.5: A cell-encoded peptide library, a medium comprising >80% organic solvent, a GS, and a library molecule with an affinity for a GS of <1E−8 M.

A6.6.2.6: A cell-encoded peptide library, a medium comprising >80% organic solvent, a GS, and a library molecule with an affinity for a GS of <1E−9 M.

A6.6.2.7: A cell-encoded peptide library, a medium comprising >80% organic solvent, a GS, and a library molecule with an affinity for a GS of <1E−10 M.

A6.6.2.8: A cell-encoded peptide library, a medium comprising >80% organic solvent, a GS, and a library molecule with an affinity for a GS of <1E−12 M.

A6.6.3.1: A cell-encoded peptide library, a medium comprising >80% organic solvent, a SWNT, and a library molecule with an affinity for a SWNT of <1E−2 M.

A6.6.3.2: A cell-encoded peptide library, a medium comprising >80% organic solvent, a SWNT, and a library molecule with an affinity for a SWNT of <1E−4 M.

A6.6.3.3: A cell-encoded peptide library, a medium comprising >80% organic solvent, a SWNT, and a library molecule with an affinity for a SWNT of <1E−6 M.

A6.6.3.4: A cell-encoded peptide library, a medium comprising >80% organic solvent, a SWNT, and a library molecule with an affinity for a SWNT of <1E−7 M.

A6.6.3.5: A cell-encoded peptide library, a medium comprising >80% organic solvent, a SWNT, and a library molecule with an affinity for a SWNT of <1E−8 M.

A6.6.3.6: A cell-encoded peptide library, a medium comprising >80% organic solvent, a SWNT, and a library molecule with an affinity for a SWNT of <1E−9 M.

A6.6.3.7: A cell-encoded peptide library, a medium comprising >80% organic solvent, a SWNT, and a library molecule with an affinity for a SWNT of <1E−10 M.

A6.6.3.8: A cell-encoded peptide library, a medium comprising >80% organic solvent, a SWNT, and a library molecule with an affinity for a SWNT of <1E−12 M.

A6.6.4.1: A cell-encoded peptide library, a medium comprising >80% organic solvent, a MWNT, and a library molecule with an affinity for a MWNT of <1E−2 M.

A6.6.4.2: A cell-encoded peptide library, a medium comprising >80% organic solvent, a MWNT, and a library molecule with an affinity for a MWNT of <1E−4 M.

A6.6.4.3: A cell-encoded peptide library, a medium comprising >80% organic solvent, a MWNT, and a library molecule with an affinity for a MWNT of <1E−6 M.

A6.6.4.4: A cell-encoded peptide library, a medium comprising >80% organic solvent, a MWNT, and a library molecule with an affinity for a MWNT of <1E−7 M.

A6.6.4.5: A cell-encoded peptide library, a medium comprising >80% organic solvent, a MWNT, and a library molecule with an affinity for a MWNT of <1E−8 M.

A6.6.4.6: A cell-encoded peptide library, a medium comprising >80% organic solvent, a MWNT, and a library molecule with an affinity for a MWNT of <1E−9 M.

A6.6.4.7: A cell-encoded peptide library, a medium comprising >80% organic solvent, a MWNT, and a library molecule with an affinity for a MWNT of <1E−10 M.

A6.6.4.8: A cell-encoded peptide library, a medium comprising >80% organic solvent, a MWNT, and a library molecule with an affinity for a MWNT of <1E−12 M.

A6.6.5.1: A cell-encoded peptide library, a medium comprising >80% organic solvent, a nanotube, and a library molecule with an affinity for a nanotube of <1E−2 M.

A6.6.5.2: A cell-encoded peptide library, a medium comprising >80% organic solvent, a nanotube, and a library molecule with an affinity for a nanotube of <1E−4 M.

A6.6.5.3: A cell-encoded peptide library, a medium comprising >80% organic solvent, a nanotube, and a library molecule with an affinity for a nanotube of <1E−6 M.

A6.6.5.4: A cell-encoded peptide library, a medium comprising >80% organic solvent, a nanotube, and a library molecule with an affinity for a nanotube of <1E−7 M.

A6.6.5.5: A cell-encoded peptide library, a medium comprising >80% organic solvent, a nanotube, and a library molecule with an affinity for a nanotube of <1E−8 M.

A6.6.5.6: A cell-encoded peptide library, a medium comprising >80% organic solvent, a nanotube, and a library molecule with an affinity for a nanotube of <1E−9 M.

A6.6.5.7: A cell-encoded peptide library, a medium comprising >80% organic solvent, a nanotube, and a library molecule with an affinity for a nanotube of <1E−10 M.

A6.6.5.8: A cell-encoded peptide library, a medium comprising >80% organic solvent, a nanotube, and a library molecule with an affinity for a nanotube of <1E−12 M.

A6.6.6.1: A cell-encoded peptide library, a medium comprising >80% organic solvent, a fullerene, and a library molecule with an affinity for a fullerene of <1E−2 M.

A6.6.6.2: A cell-encoded peptide library, a medium comprising >80% organic solvent, a fullerene, and a library molecule with an affinity for a fullerene of <1E−4 M.

A6.6.6.3: A cell-encoded peptide library, a medium comprising >80% organic solvent, a fullerene, and a library molecule with an affinity for a fullerene of <1E−6 M.

A6.6.6.4: A cell-encoded peptide library, a medium comprising >80% organic solvent, a fullerene, and a library molecule with an affinity for a fullerene of <1E−7 M.

A6.6.6.5: A cell-encoded peptide library, a medium comprising >80% organic solvent, a fullerene, and a library molecule with an affinity for a fullerene of <1E−8 M.

A6.6.6.6: A cell-encoded peptide library, a medium comprising >80% organic solvent, a fullerene, and a library molecule with an affinity for a fullerene of <1E−9 M.

A6.6.6.7: A cell-encoded peptide library, a medium comprising >80% organic solvent, a fullerene, and a library molecule with an affinity for a fullerene of <1E−10 M.

A6.6.6.8: A cell-encoded peptide library, a medium comprising >80% organic solvent, a fullerene, and a library molecule with an affinity for a fullerene of <1E−12 M.

A6.6.7.1: A cell-encoded peptide library, a medium comprising >80% organic solvent, a BNNT, and a library molecule with an affinity for a BNNT of <1E−2 M.

A6.6.7.2: A cell-encoded peptide library, a medium comprising >80% organic solvent, a BNNT, and a library molecule with an affinity for a BNNT of <1E−4 M.

A6.6.7.3: A cell-encoded peptide library, a medium comprising >80% organic solvent, a BNNT, and a library molecule with an affinity for a BNNT of <1E−6 M.

A6.6.7.4: A cell-encoded peptide library, a medium comprising >80% organic solvent, a BNNT, and a library molecule with an affinity for a BNNT of <1E−7 M.

A6.6.7.5: A cell-encoded peptide library, a medium comprising >80% organic solvent, a BNNT, and a library molecule with an affinity for a BNNT of <1E−8 M.

A6.6.7.6: A cell-encoded peptide library, a medium comprising >80% organic solvent, a BNNT, and a library molecule with an affinity for a BNNT of <1E−9 M.

A6.6.7.7: A cell-encoded peptide library, a medium comprising >80% organic solvent, a BNNT, and a library molecule with an affinity for a BNNT of <1E−10 M.

A6.6.7.8: A cell-encoded peptide library, a medium comprising >80% organic solvent, a BNNT, and a library molecule with an affinity for a BNNT of <1E−12 M.

A6.7.1.1: A cell-encoded peptide library, a medium comprising >80% organic solvent and a soluble polymer, a CNT, and a library molecule with an affinity for a CNT of <1E−2 M.

A6.7.1.2: A cell-encoded peptide library, a medium comprising >80% organic solvent and a soluble polymer, a CNT, and a library molecule with an affinity for a CNT of <1E−4 M.

A6.7.1.3: A cell-encoded peptide library, a medium comprising >80% organic solvent and a soluble polymer, a CNT, and a library molecule with an affinity for a CNT of <1E−6 M.

A6.7.1.4: A cell-encoded peptide library, a medium comprising >80% organic solvent and a soluble polymer, a CNT, and a library molecule with an affinity for a CNT of <1E−7 M.

A6.7.1.5: A cell-encoded peptide library, a medium comprising >80% organic solvent and a soluble polymer, a CNT, and a library molecule with an affinity for a CNT of <1E−8 M.

A6.7.1.6: A cell-encoded peptide library, a medium comprising >80% organic solvent and a soluble polymer, a CNT, and a library molecule with an affinity for a CNT of <1E−9 M.

A6.7.1.7: A cell-encoded peptide library, a medium comprising >80% organic solvent and a soluble polymer, a CNT, and a library molecule with an affinity for a CNT of <1E−10 M.

A6.7.1.8: A cell-encoded peptide library, a medium comprising >80% organic solvent and a soluble polymer, a CNT, and a library molecule with an affinity for a CNT of <1E−12 M.

A6.7.2.1: A cell-encoded peptide library, a medium comprising >80% organic solvent and a soluble polymer, a GS, and a library molecule with an affinity for a GS of <1E−2 M.

A6.7.2.2: A cell-encoded peptide library, a medium comprising >80% organic solvent and a soluble polymer, a GS, and a library molecule with an affinity for a GS of <1E−4 M.

A6.7.2.3: A cell-encoded peptide library, a medium comprising >80% organic solvent and a soluble polymer, a GS, and a library molecule with an affinity for a GS of <1E−6 M.

A6.7.2.4: A cell-encoded peptide library, a medium comprising >80% organic solvent and a soluble polymer, a GS, and a library molecule with an affinity for a GS of <1E−7 M.

A6.7.2.5: A cell-encoded peptide library, a medium comprising >80% organic solvent and a soluble polymer, a GS, and a library molecule with an affinity for a GS of <1E−8 M.

A6.7.2.6: A cell-encoded peptide library, a medium comprising >80% organic solvent and a soluble polymer, a GS, and a library molecule with an affinity for a GS of <1E−9 M.

A6.7.2.7: A cell-encoded peptide library, a medium comprising >80% organic solvent and a soluble polymer, a GS, and a library molecule with an affinity for a GS of <1E−10 M.

A6.7.2.8: A cell-encoded peptide library, a medium comprising >80% organic solvent and a soluble polymer, a GS, and a library molecule with an affinity for a GS of <1E−12 M.

A6.7.3.1: A cell-encoded peptide library, a medium comprising >80% organic solvent and a soluble polymer, a SWNT, and a library molecule with an affinity for a SWNT of <1E−2 M.

A6.7.3.2: A cell-encoded peptide library, a medium comprising >80% organic solvent and a soluble polymer, a SWNT, and a library molecule with an affinity for a SWNT of <1E−4 M.

A6.7.3.3: A cell-encoded peptide library, a medium comprising >80% organic solvent and a soluble polymer, a SWNT, and a library molecule with an affinity for a SWNT of <1E−6 M.

A6.7.3.4: A cell-encoded peptide library, a medium comprising >80% organic solvent and a soluble polymer, a SWNT, and a library molecule with an affinity for a SWNT of <1E−7 M.

A6.7.3.5: A cell-encoded peptide library, a medium comprising >80% organic solvent and a soluble polymer, a SWNT, and a library molecule with an affinity for a SWNT of <1E−8 M.

A6.7.3.6: A cell-encoded peptide library, a medium comprising >80% organic solvent and a soluble polymer, a SWNT, and a library molecule with an affinity for a SWNT of <1E−9 M.

A6.7.3.7: A cell-encoded peptide library, a medium comprising >80% organic solvent and a soluble polymer, a SWNT, and a library molecule with an affinity for a SWNT of <1E−10 M.

A6.7.3.8: A cell-encoded peptide library, a medium comprising >80% organic solvent and a soluble polymer, a SWNT, and a library molecule with an affinity for a SWNT of <1E−12 M.

A6.7.4.1: A cell-encoded peptide library, a medium comprising >80% organic solvent and a soluble polymer, a MWNT, and a library molecule with an affinity for a MWNT of <1E−2 M.

A6.7.4.2: A cell-encoded peptide library, a medium comprising >80% organic solvent and a soluble polymer, a MWNT, and a library molecule with an affinity for a MWNT of <1E−4 M.

A6.7.4.3: A cell-encoded peptide library, a medium comprising >80% organic solvent and a soluble polymer, a MWNT, and a library molecule with an affinity for a MWNT of <1E−6 M.

A6.7.4.4: A cell-encoded peptide library, a medium comprising >80% organic solvent and a soluble polymer, a MWNT, and a library molecule with an affinity for a MWNT of <1E−7 M.

A6.7.4.5: A cell-encoded peptide library, a medium comprising >80% organic solvent and a soluble polymer, a MWNT, and a library molecule with an affinity for a MWNT of <1E−8 M.

A6.7.4.6: A cell-encoded peptide library, a medium comprising >80% organic solvent and a soluble polymer, a MWNT, and a library molecule with an affinity for a MWNT of <1E−9 M.

A6.7.4.7: A cell-encoded peptide library, a medium comprising >80% organic solvent and a soluble polymer, a MWNT, and a library molecule with an affinity for a MWNT of <1E−10 M.

A6.7.4.8: A cell-encoded peptide library, a medium comprising >80% organic solvent and a soluble polymer, a MWNT, and a library molecule with an affinity for a MWNT of <1E−12 M.

A6.7.5.1: A cell-encoded peptide library, a medium comprising >80% organic solvent and a soluble polymer, a nanotube, and a library molecule with an affinity for a nanotube of <1E−2 M.

A6.7.5.2: A cell-encoded peptide library, a medium comprising >80% organic solvent and a soluble polymer, a nanotube, and a library molecule with an affinity for a nanotube of <1E−4 M.

A6.7.5.3: A cell-encoded peptide library, a medium comprising >80% organic solvent and a soluble polymer, a nanotube, and a library molecule with an affinity for a nanotube of <1E−6 M.

A6.7.5.4: A cell-encoded peptide library, a medium comprising >80% organic solvent and a soluble polymer, a nanotube, and a library molecule with an affinity for a nanotube of <1E−7 M.

A6.7.5.5: A cell-encoded peptide library, a medium comprising >80% organic solvent and a soluble polymer, a nanotube, and a library molecule with an affinity for a nanotube of <1E−8 M.

A6.7.5.6: A cell-encoded peptide library, a medium comprising >80% organic solvent and a soluble polymer, a nanotube, and a library molecule with an affinity for a nanotube of <1E−9 M.

A6.7.5.7: A cell-encoded peptide library, a medium comprising >80% organic solvent and a soluble polymer, a nanotube, and a library molecule with an affinity for a nanotube of <1E−10 M.

A6.7.5.8: A cell-encoded peptide library, a medium comprising >80% organic solvent and a soluble polymer, a nanotube, and a library molecule with an affinity for a nanotube of <1E−12 M.

A6.7.6.1: A cell-encoded peptide library, a medium comprising >80% organic solvent and a soluble polymer, a fullerene, and a library molecule with an affinity for a fullerene of <1E−2 M.

A6.7.6.2: A cell-encoded peptide library, a medium comprising >80% organic solvent and a soluble polymer, a fullerene, and a library molecule with an affinity for a fullerene of <1E−4 M.

A6.7.6.3: A cell-encoded peptide library, a medium comprising >80% organic solvent and a soluble polymer, a fullerene, and a library molecule with an affinity for a fullerene of <1E−6 M.

A6.7.6.4: A cell-encoded peptide library, a medium comprising >80% organic solvent and a soluble polymer, a fullerene, and a library molecule with an affinity for a fullerene of <1E−7 M.

A6.7.6.5: A cell-encoded peptide library, a medium comprising >80% organic solvent and a soluble polymer, a fullerene, and a library molecule with an affinity for a fullerene of <1E−8 M.

A6.7.6.6: A cell-encoded peptide library, a medium comprising >80% organic solvent and a soluble polymer, a fullerene, and a library molecule with an affinity for a fullerene of <1E−9 M.

A6.7.6.7: A cell-encoded peptide library, a medium comprising >80% organic solvent and a soluble polymer, a fullerene, and a library molecule with an affinity for a fullerene of <1E−10 M.

A6.7.6.8: A cell-encoded peptide library, a medium comprising >80% organic solvent and a soluble polymer, a fullerene, and a library molecule with an affinity for a fullerene of <1E−12 M.

A6.7.7.1: A cell-encoded peptide library, a medium comprising >80% organic solvent and a soluble polymer, a BNNT, and a library molecule with an affinity for a BNNT of <1E−2 M.

A6.7.7.2: A cell-encoded peptide library, a medium comprising >80% organic solvent and a soluble polymer, a BNNT, and a library molecule with an affinity for a BNNT of <1E−4 M.

A6.7.7.3: A cell-encoded peptide library, a medium comprising >80% organic solvent and a soluble polymer, a BNNT, and a library molecule with an affinity for a BNNT of <1E−6 M.

A6.7.7.4: A cell-encoded peptide library, a medium comprising >80% organic solvent and a soluble polymer, a BNNT, and a library molecule with an affinity for a BNNT of <1E−7 M.

A6.7.7.5: A cell-encoded peptide library, a medium comprising >80% organic solvent and a soluble polymer, a BNNT, and a library molecule with an affinity for a BNNT of <1E−8 M.

A6.7.7.6: A cell-encoded peptide library, a medium comprising >80% organic solvent and a soluble polymer, a BNNT, and a library molecule with an affinity for a BNNT of <1E−9 M.

A6.7.7.7: A cell-encoded peptide library, a medium comprising >80% organic solvent and a soluble polymer, a BNNT, and a library molecule with an affinity for a BNNT of <1E−10 M.

A6.7.7.8: A cell-encoded peptide library, a medium comprising >80% organic solvent and a soluble polymer, a BNNT, and a library molecule with an affinity for a BNNT of <1E−12 M.

A6.8.1.1: A cell-encoded peptide library, a medium comprising >95% organic solvent, a CNT, and a library molecule with an affinity for a CNT of <1E−2 M.

A6.8.1.2: A cell-encoded peptide library, a medium comprising >95% organic solvent, a CNT, and a library molecule with an affinity for a CNT of <1E−4 M.

A6.8.1.3: A cell-encoded peptide library, a medium comprising >95% organic solvent, a CNT, and a library molecule with an affinity for a CNT of <1E−6 M.

A6.8.1.4: A cell-encoded peptide library, a medium comprising >95% organic solvent, a CNT, and a library molecule with an affinity for a CNT of <1E−7 M.

A6.8.1.5: A cell-encoded peptide library, a medium comprising >95% organic solvent, a CNT, and a library molecule with an affinity for a CNT of <1E−8 M.

A6.8.1.6: A cell-encoded peptide library, a medium comprising >95% organic solvent, a CNT, and a library molecule with an affinity for a CNT of <1E−9 M.

A6.8.1.7: A cell-encoded peptide library, a medium comprising >95% organic solvent, a CNT, and a library molecule with an affinity for a CNT of <1E−10 M.

A6.8.1.8: A cell-encoded peptide library, a medium comprising >95% organic solvent, a CNT, and a library molecule with an affinity for a CNT of <1E−12 M.

A6.8.2.1: A cell-encoded peptide library, a medium comprising >95% organic solvent, a GS, and a library molecule with an affinity for a GS of <1E−2 M.

A6.8.2.2: A cell-encoded peptide library, a medium comprising >95% organic solvent, a GS, and a library molecule with an affinity for a GS of <1E−4 M.

A6.8.2.3: A cell-encoded peptide library, a medium comprising >95% organic solvent, a GS, and a library molecule with an affinity for a GS of <1E−6 M.

A6.8.2.4: A cell-encoded peptide library, a medium comprising >95% organic solvent, a GS, and a library molecule with an affinity for a GS of <1E−7 M.

A6.8.2.5: A cell-encoded peptide library, a medium comprising >95% organic solvent, a GS, and a library molecule with an affinity for a GS of <1E−8 M.

A6.8.2.6: A cell-encoded peptide library, a medium comprising >95% organic solvent, a GS, and a library molecule with an affinity for a GS of <1E−9 M.

A6.8.2.7: A cell-encoded peptide library, a medium comprising >95% organic solvent, a GS, and a library molecule with an affinity for a GS of <1E−10 M.

A6.8.2.8: A cell-encoded peptide library, a medium comprising >95% organic solvent, a GS, and a library molecule with an affinity for a GS of <1E−12 M.

A6.8.3.1: A cell-encoded peptide library, a medium comprising >95% organic solvent, a SWNT, and a library molecule with an affinity for a SWNT of <1E−2 M.

A6.8.3.2: A cell-encoded peptide library, a medium comprising >95% organic solvent, a SWNT, and a library molecule with an affinity for a SWNT of <1E−4 M.

A6.8.3.3: A cell-encoded peptide library, a medium comprising >95% organic solvent, a SWNT, and a library molecule with an affinity for a SWNT of <1E−6 M.

A6.8.3.4: A cell-encoded peptide library, a medium comprising >95% organic solvent, a SWNT, and a library molecule with an affinity for a SWNT of <1E−7 M.

A6.8.3.5: A cell-encoded peptide library, a medium comprising >95% organic solvent, a SWNT, and a library molecule with an affinity for a SWNT of <1E−8 M.

A6.8.3.6: A cell-encoded peptide library, a medium comprising >95% organic solvent, a SWNT, and a library molecule with an affinity for a SWNT of <1E−9 M.

A6.8.3.7: A cell-encoded peptide library, a medium comprising >95% organic solvent, a SWNT, and a library molecule with an affinity for a SWNT of <1E−10 M.

A6.8.3.8: A cell-encoded peptide library, a medium comprising >95% organic solvent, a SWNT, and a library molecule with an affinity for a SWNT of <1E−12 M.

A6.8.4.1: A cell-encoded peptide library, a medium comprising >95% organic solvent, a MWNT, and a library molecule with an affinity for a MWNT of <1E−2 M.

A6.8.4.2: A cell-encoded peptide library, a medium comprising >95% organic solvent, a MWNT, and a library molecule with an affinity for a MWNT of <1E−4 M.

A6.8.4.3: A cell-encoded peptide library, a medium comprising >95% organic solvent, a MWNT, and a library molecule with an affinity for a MWNT of <1E−6 M.

A6.8.4.4: A cell-encoded peptide library, a medium comprising >95% organic solvent, a MWNT, and a library molecule with an affinity for a MWNT of <1E−7 M.

A6.8.4.5: A cell-encoded peptide library, a medium comprising >95% organic solvent, a MWNT, and a library molecule with an affinity for a MWNT of <1E−8 M.

A6.8.4.6: A cell-encoded peptide library, a medium comprising >95% organic solvent, a MWNT, and a library molecule with an affinity for a MWNT of <1E−9 M.

A6.8.4.7: A cell-encoded peptide library, a medium comprising >95% organic solvent, a MWNT, and a library molecule with an affinity for a MWNT of <1E−10 M.

A6.8.4.8: A cell-encoded peptide library, a medium comprising >95% organic solvent, a MWNT, and a library molecule with an affinity for a MWNT of <1E−12 M.

A6.8.5.1: A cell-encoded peptide library, a medium comprising >95% organic solvent, a nanotube, and a library molecule with an affinity for a nanotube of <1E−2 M.

A6.8.5.2: A cell-encoded peptide library, a medium comprising >95% organic solvent, a nanotube, and a library molecule with an affinity for a nanotube of <1E−4 M.

A6.8.5.3: A cell-encoded peptide library, a medium comprising >95% organic solvent, a nanotube, and a library molecule with an affinity for a nanotube of <1E−6 M.

A6.8.5.4: A cell-encoded peptide library, a medium comprising >95% organic solvent, a nanotube, and a library molecule with an affinity for a nanotube of <1E−7 M.

A6.8.5.5: A cell-encoded peptide library, a medium comprising >95% organic solvent, a nanotube, and a library molecule with an affinity for a nanotube of <1E−8 M.

A6.8.5.6: A cell-encoded peptide library, a medium comprising >95% organic solvent, a nanotube, and a library molecule with an affinity for a nanotube of <1E−9 M.

A6.8.5.7: A cell-encoded peptide library, a medium comprising >95% organic solvent, a nanotube, and a library molecule with an affinity for a nanotube of <1E−10 M.

A6.8.5.8: A cell-encoded peptide library, a medium comprising >95% organic solvent, a nanotube, and a library molecule with an affinity for a nanotube of <1E−12 M.

A6.8.6.1: A cell-encoded peptide library, a medium comprising >95% organic solvent, a fullerene, and a library molecule with an affinity for a fullerene of <1E−2 M.

A6.8.6.2: A cell-encoded peptide library, a medium comprising >95% organic solvent, a fullerene, and a library molecule with an affinity for a fullerene of <1E−4 M.

A6.8.6.3: A cell-encoded peptide library, a medium comprising >95% organic solvent, a fullerene, and a library molecule with an affinity for a fullerene of <1E−6 M.

A6.8.6.4: A cell-encoded peptide library, a medium comprising >95% organic solvent, a fullerene, and a library molecule with an affinity for a fullerene of <1E−7 M.

A6.8.6.5: A cell-encoded peptide library, a medium comprising >95% organic solvent, a fullerene, and a library molecule with an affinity for a fullerene of <1E−8 M.

A6.8.6.6: A cell-encoded peptide library, a medium comprising >95% organic solvent, a fullerene, and a library molecule with an affinity for a fullerene of <1E−9 M.

A6.8.6.7: A cell-encoded peptide library, a medium comprising >95% organic solvent, a fullerene, and a library molecule with an affinity for a fullerene of <1E−10 M.

A6.8.6.8: A cell-encoded peptide library, a medium comprising >95% organic solvent, a fullerene, and a library molecule with an affinity for a fullerene of <1E−12 M.

A6.8.7.1: A cell-encoded peptide library, a medium comprising >95% organic solvent, a BNNT, and a library molecule with an affinity for a BNNT of <1E−2 M.

A6.8.7.2: A cell-encoded peptide library, a medium comprising >95% organic solvent, a BNNT, and a library molecule with an affinity for a BNNT of <1E−4 M.

A6.8.7.3: A cell-encoded peptide library, a medium comprising >95% organic solvent, a BNNT, and a library molecule with an affinity for a BNNT of <1E−6 M.

A6.8.7.4: A cell-encoded peptide library, a medium comprising >95% organic solvent, a BNNT, and a library molecule with an affinity for a BNNT of <1E−7 M.

A6.8.7.5: A cell-encoded peptide library, a medium comprising >95% organic solvent, a BNNT, and a library molecule with an affinity for a BNNT of <1E−8 M.

A6.8.7.6: A cell-encoded peptide library, a medium comprising >95% organic solvent, a BNNT, and a library molecule with an affinity for a BNNT of <1E−9 M.

A6.8.7.7: A cell-encoded peptide library, a medium comprising >95% organic solvent, a BNNT, and a library molecule with an affinity for a BNNT of <1E−10 M.

A6.8.7.8: A cell-encoded peptide library, a medium comprising >95% organic solvent, a BNNT, and a library molecule with an affinity for a BNNT of <1E−12 M.

A6.9.1.1: A cell-encoded peptide library, a medium comprising >95% organic solvent and a soluble polymer, a CNT, and a library molecule with an affinity for a CNT of <1E−2 M.

A6.9.1.2: A cell-encoded peptide library, a medium comprising >95% organic solvent and a soluble polymer, a CNT, and a library molecule with an affinity for a CNT of <1E−4 M.

A6.9.1.3: A cell-encoded peptide library, a medium comprising >95% organic solvent and a soluble polymer, a CNT, and a library molecule with an affinity for a CNT of <1E−6 M.

A6.9.1.4: A cell-encoded peptide library, a medium comprising >95% organic solvent and a soluble polymer, a CNT, and a library molecule with an affinity for a CNT of <1E−7 M.

A6.9.1.5: A cell-encoded peptide library, a medium comprising >95% organic solvent and a soluble polymer, a CNT, and a library molecule with an affinity for a CNT of <1E−8 M.

A6.9.1.6: A cell-encoded peptide library, a medium comprising >95% organic solvent and a soluble polymer, a CNT, and a library molecule with an affinity for a CNT of <1E−9 M.

A6.9.1.7: A cell-encoded peptide library, a medium comprising >95% organic solvent and a soluble polymer, a CNT, and a library molecule with an affinity for a CNT of <1E−10 M.

A6.9.1.8: A cell-encoded peptide library, a medium comprising >95% organic solvent and a soluble polymer, a CNT, and a library molecule with an affinity for a CNT of <1E−12 M.

A6.9.2.1: A cell-encoded peptide library, a medium comprising >95% organic solvent and a soluble polymer, a GS, and a library molecule with an affinity for a GS of <1E−2 M.

A6.9.2.2: A cell-encoded peptide library, a medium comprising >95% organic solvent and a soluble polymer, a GS, and a library molecule with an affinity for a GS of <1E−4 M.

A6.9.2.3: A cell-encoded peptide library, a medium comprising >95% organic solvent and a soluble polymer, a GS, and a library molecule with an affinity for a GS of <1E−6 M.

A6.9.2.4: A cell-encoded peptide library, a medium comprising >95% organic solvent and a soluble polymer, a GS, and a library molecule with an affinity for a GS of <1E−7 M.

A6.9.2.5: A cell-encoded peptide library, a medium comprising >95% organic solvent and a soluble polymer, a GS, and a library molecule with an affinity for a GS of <1E−8 M.

A6.9.2.6: A cell-encoded peptide library, a medium comprising >95% organic solvent and a soluble polymer, a GS, and a library molecule with an affinity for a GS of <1E−9 M.

A6.9.2.7: A cell-encoded peptide library, a medium comprising >95% organic solvent and a soluble polymer, a GS, and a library molecule with an affinity for a GS of <1E−10 M.

A6.9.2.8: A cell-encoded peptide library, a medium comprising >95% organic solvent and a soluble polymer, a GS, and a library molecule with an affinity for a GS of <1E−12 M.

A6.9.3.1: A cell-encoded peptide library, a medium comprising >95% organic solvent and a soluble polymer, a SWNT, and a library molecule with an affinity for a SWNT of <1E−2 M.

A6.9.3.2: A cell-encoded peptide library, a medium comprising >95% organic solvent and a soluble polymer, a SWNT, and a library molecule with an affinity for a SWNT of <1E−4 M.

A6.9.3.3: A cell-encoded peptide library, a medium comprising >95% organic solvent and a soluble polymer, a SWNT, and a library molecule with an affinity for a SWNT of <1E−6 M.

A6.9.3.4: A cell-encoded peptide library, a medium comprising >95% organic solvent and a soluble polymer, a SWNT, and a library molecule with an affinity for a SWNT of <1E−7 M.

A6.9.3.5: A cell-encoded peptide library, a medium comprising >95% organic solvent and a soluble polymer, a SWNT, and a library molecule with an affinity for a SWNT of <1E−8 M.

A6.9.3.6: A cell-encoded peptide library, a medium comprising >95% organic solvent and a soluble polymer, a SWNT, and a library molecule with an affinity for a SWNT of <1E−9 M.

A6.9.3.7: A cell-encoded peptide library, a medium comprising >95% organic solvent and a soluble polymer, a SWNT, and a library molecule with an affinity for a SWNT of <1E−10 M.

A6.9.3.8: A cell-encoded peptide library, a medium comprising >95% organic solvent and a soluble polymer, a SWNT, and a library molecule with an affinity for a SWNT of <1E−12 M.

A6.9.4.1: A cell-encoded peptide library, a medium comprising >95% organic solvent and a soluble polymer, a MWNT, and a library molecule with an affinity for a MWNT of <1E−2 M.

A6.9.4.2: A cell-encoded peptide library, a medium comprising >95% organic solvent and a soluble polymer, a MWNT, and a library molecule with an affinity for a MWNT of <1E−4 M.

A6.9.4.3: A cell-encoded peptide library, a medium comprising >95% organic solvent and a soluble polymer, a MWNT, and a library molecule with an affinity for a MWNT of <1E−6 M.

A6.9.4.4: A cell-encoded peptide library, a medium comprising >95% organic solvent and a soluble polymer, a MWNT, and a library molecule with an affinity for a MWNT of <1E−7 M.

A6.9.4.5: A cell-encoded peptide library, a medium comprising >95% organic solvent and a soluble polymer, a MWNT, and a library molecule with an affinity for a MWNT of <1E−8 M.

A6.9.4.6: A cell-encoded peptide library, a medium comprising >95% organic solvent and a soluble polymer, a MWNT, and a library molecule with an affinity for a MWNT of <1E−9 M.

A6.9.4.7: A cell-encoded peptide library, a medium comprising >95% organic solvent and a soluble polymer, a MWNT, and a library molecule with an affinity for a MWNT of <1E−10 M.

A6.9.4.8: A cell-encoded peptide library, a medium comprising >95% organic solvent and a soluble polymer, a MWNT, and a library molecule with an affinity for a MWNT of <1E−12 M.

A6.9.5.1: A cell-encoded peptide library, a medium comprising >95% organic solvent and a soluble polymer, a nanotube, and a library molecule with an affinity for a nanotube of <1E−2 M.

A6.9.5.2: A cell-encoded peptide library, a medium comprising >95% organic solvent and a soluble polymer, a nanotube, and a library molecule with an affinity for a nanotube of <1E−4 M.

A6.9.5.3: A cell-encoded peptide library, a medium comprising >95% organic solvent and a soluble polymer, a nanotube, and a library molecule with an affinity for a nanotube of <1E−6 M.

A6.9.5.4: A cell-encoded peptide library, a medium comprising >95% organic solvent and a soluble polymer, a nanotube, and a library molecule with an affinity for a nanotube of <1E−7 M.

A6.9.5.5: A cell-encoded peptide library, a medium comprising >95% organic solvent and a soluble polymer, a nanotube, and a library molecule with an affinity for a nanotube of <1E−8 M.

A6.9.5.6: A cell-encoded peptide library, a medium comprising >95% organic solvent and a soluble polymer, a nanotube, and a library molecule with an affinity for a nanotube of <1E−9 M.

A6.9.5.7: A cell-encoded peptide library, a medium comprising >95% organic solvent and a soluble polymer, a nanotube, and a library molecule with an affinity for a nanotube of <1E−10 M.

A6.9.5.8: A cell-encoded peptide library, a medium comprising >95% organic solvent and a soluble polymer, a nanotube, and a library molecule with an affinity for a nanotube of <1E−12 M.

A6.9.6.1: A cell-encoded peptide library, a medium comprising >95% organic solvent and a soluble polymer, a fullerene, and a library molecule with an affinity for a fullerene of <1E−2 M.

A6.9.6.2: A cell-encoded peptide library, a medium comprising >95% organic solvent and a soluble polymer, a fullerene, and a library molecule with an affinity for a fullerene of <1E−4 M.

A6.9.6.3: A cell-encoded peptide library, a medium comprising >95% organic solvent and a soluble polymer, a fullerene, and a library molecule with an affinity for a fullerene of <1E−6 M.

A6.9.6.4: A cell-encoded peptide library, a medium comprising >95% organic solvent and a soluble polymer, a fullerene, and a library molecule with an affinity for a fullerene of <1E−7 M.

A6.9.6.5: A cell-encoded peptide library, a medium comprising >95% organic solvent and a soluble polymer, a fullerene, and a library molecule with an affinity for a fullerene of <1E−8 M.

A6.9.6.6: A cell-encoded peptide library, a medium comprising >95% organic solvent and a soluble polymer, a fullerene, and a library molecule with an affinity for a fullerene of <1E−9 M.

A6.9.6.7: A cell-encoded peptide library, a medium comprising >95% organic solvent and a soluble polymer, a fullerene, and a library molecule with an affinity for a fullerene of <1E−10 M.

A6.9.6.8: A cell-encoded peptide library, a medium comprising >95% organic solvent and a soluble polymer, a fullerene, and a library molecule with an affinity for a fullerene of <1E−12 M.

A6.9.7.1: A cell-encoded peptide library, a medium comprising >95% organic solvent and a soluble polymer, a BNNT, and a library molecule with an affinity for a BNNT of <1E−2 M.

A6.9.7.2: A cell-encoded peptide library, a medium comprising >95% organic solvent and a soluble polymer, a BNNT, and a library molecule with an affinity for a BNNT of <1E−4 M.

A6.9.7.3: A cell-encoded peptide library, a medium comprising >95% organic solvent and a soluble polymer, a BNNT, and a library molecule with an affinity for a BNNT of <1E−6 M.

A6.9.7.4: A cell-encoded peptide library, a medium comprising >95% organic solvent and a soluble polymer, a BNNT, and a library molecule with an affinity for a BNNT of <1E−7 M.

A6.9.7.5: A cell-encoded peptide library, a medium comprising >95% organic solvent and a soluble polymer, a BNNT, and a library molecule with an affinity for a BNNT of <1E−8 M.

A6.9.7.6: A cell-encoded peptide library, a medium comprising >95% organic solvent and a soluble polymer, a BNNT, and a library molecule with an affinity for a BNNT of <1E−9 M.

A6.9.7.7: A cell-encoded peptide library, a medium comprising >95% organic solvent and a soluble polymer, a BNNT, and a library molecule with an affinity for a BNNT of <1E−10 M.

A6.9.7.8: A cell-encoded peptide library, a medium comprising >95% organic solvent and a soluble polymer, a BNNT, and a library molecule with an affinity for a BNNT of <1E−12 M.

### EXAMPLES

The following section presents examples which illustrate but do not limit the present invention. Libraries of molecules according to the present invention may contain any type of molecules and molecule fragments. In a preferred embodiment, libraries of molecules comprise drug-like fragments.

In another preferred embodiment, libraries of molecules comprise non-drug-like fragments, such as charged fragments or flexible fragments.

The following table describes molecules suitable for the present invention (left column) and corresponding materials to which said molecules can bind (middle column):

| Molecule | Material | Reference^{a} |
|---|---|---|
| (SVSVGMKPSPRPGGGK) | Hydroxyappatite | Weiger et al. 2010 |
| (KD = 14.1 μm ± 3.8 μm) to HA | | |
| THRTSTLDYFVI | Chlorine-doped | Sanghvi et al. 2005 |
| | polypyrrole (PPyCl). | |
| benzene, naphthalene, biphenyl, fluorene, phenanthrene, | Carbon nanotubes | Yoo et al. 2011 |
| anthracene, pyrene, triphenylene, p-terphenyl, and | | |
| tetraphene | | |
| Pyrenecarboxylic acid, | Carbon nanotubes | Simmons et al. 2009 |
| SDS, simple alkyl chains of surfactants such asSDS, | Carbon nanotubes | Hu et al. 2009 |
| SDSA (sodium dodecyl sulfonate), DTAB | | |
| (dodecyltrimethylammonium | | |
| bromide), NaDDBS, Tween-60 and Tween-80 | | |
| (polyethylene oxide (20) sorbitan | | |
| monostearate and monooleate, PSPEO, polyvinyl | | |
| pyrrolidone (PVP) and polystyrene | | |
| sulfonate (PSS), poly(2-ethyl-2-oxazoline) (PEOX), | | |
| Poly (maleicacid/octyl vinyl ether) (PMAOVE), | | |
| poly(m-phenylenevinylene) (PmPV), sulfonated | | |
| polyaniline (SPAN), poly(phenylacetylene)-wrapped | | |
| carbon nanotubes | | |
| (PPA-CNTs), Lysophospholipids, Lysozyme, | | |
| porphyrins and phthalocyanins, zinc | | |
| protoporphyrin IX | | |
| Palladium, platinum, and titanium particles | | Park et al. 2009 |

^{a}References; Weiger et al. Biomaterials Volume 31, Issue 11, April 2010, Pages 2955-2963, Sanghvi et al. 2005, Nature Materials 4, 496-502 (2005); Yoo et al. Nanoscale, 2011, 3, 2517-2522; Simmons et al. ACS Nano, 2009, 3 (4), pp 865-870; Hu et al., J. Chin. Chem. Soc., Vol. 56, No. 2, 2009 and references therein; Park et al. Appl. Phys. Lett. 94, 073105 (2009);

Other suitable molecules comprise; Thiols (which can react with metal surfaces), Congo Red, porphyrins, phthalocyanines, polystyrene sulphonate (PSS), polyvinyl pyrrolidone (PVP), poly(m-phenylenevinylene) substituted with octyloxy chains, riboflavin, ionic and nonionic surfactans, such as sodium dodecyl, sulfate (SDS), sodium dodecylbenzene sulfonate (SDBS), cetyltrimethylammonium bromide (CTAB), Brij, Tween, Triton X and Siloxane polyether copolymer (PSPEO). SDSA (sodium dodecyl sulfonate), DTAB (dodecyltrimethylammonium bromide), PSPEO, polyvinyl pyrrolidone (PVP) and polystyrene sulfonate (PSS), poly(2-ethyl-2-oxazoline) (PEOX), Poly (maleic acid/octyl vinyl ether) (PMAOVE), poly(m-phenylenevinylene) (PmPV), sulfonated polyaniline (SPAN), fluorescein-polyethylene glycol, maleimide polymers.

### Examples of Identification of Molecules According to the Present Invention

### Example 1.1.1.1 Identification of Peptides with Affinity Towards Carbon Nanotubes

In this example library molecules (M) are peptides linked by bacteria (L) to DNA tags encoding the peptide. The material used is carbon nanotubes and the medium used is PKT buffer (10 mM potassium phosphate, pH 7.0, 0.1 M KCl, 1% Triton X-100) and TNT buffer (TNT buffer is 10 mM Tris-HCl, pH 7.8, 10 mM NaCl, 1% Triton X-100). Identification of carbon nanotube binding molecules is described in Brown et al. (Small. 2008 April; 4 (4):416-20). The CNT target is prepared from single-walled carbon nanotubes (purified HiPCO, Carbon Nanotechnologies Inc., US) used as purchased. Bundles and individual tubes are suspended in dichloroethane by sonication and deposited on pieces of double side polished Si wafers. The CNT mats are inspected by scanning electron microscopy. A uniform, dense coverage on both sides of the 1 cm2 substrates is achieved by repeatedly depositing drops from the suspension, rinsing with isopropanol, and blow drying with nitrogen. Test enrichments are conducted inn a phosphate buffer (PKT). The enrichment for adhesion to CNT mats using solution conditions is done similarly to other searches for CNT binders. The population recovered from a single cycle of enrichment for binding to CNTs in a Tris-HCl buffer (TNT) differed from that obtained in PKT.

### Example 2.1.1.1 Identification of Peptides with Affinity Towards Multi-Walled Carbon Nanotubes

In this example library molecules (M) are peptides linked by bacteriophages (L) to DNA tags encoding the peptide. The material used is multi-walled carbon nanotubes and the medium used is 150 mM Tris-buffered-saline with varying concentrations of Tween 20. Identification of multi-walled carbon nanotube (MWCNT) binding molecules is described in Wang et al. (Nature Materials 2, 196-200 (2003) High-purity α-SiC powder of 10.1 μm mean particle diameter (Norton Company) is heated in high-purity graphite crucibles to 1,700° C. for 24 h in a graphite-heated furnace under mild vacuum (0.1-1 torr). This procedure results in a high rate of SiO volatilization because of the modest vacuum, while pinning the local carbon activity in the graphite stability regime because of the buffered atmosphere provided by the graphite crucibles. The slightly oxidizing ambient results in nucleation of nanotubes at the SiC surface followed by complete conversion to densely packed arrays of multi-walled nanotubes (MWNTs). A few phage display experiments are also carried out using HiPCO carbon nanotubes to establish that the process will result in a controlled reduction of the quantity of bound phage, as measured by concentration of plaque-forming units (PFU). Other binding experiments, described in the following paragraphs, are done using laser-ablated or HiPCO single-walled carbon nanotubes (SWNT). The in vitro selection procedure consisted of a sequence of incubations of the phage library with the nanotubes in the presence of added detergent of varying stringency. Carbon nanotubes are suspended by sonication in 150 mM Tris-buffered-saline with varying concentration of Tween 20 (starting the first round with 0.1% at a concentration of 1 mg ml-1). Phage-display experiments are done with a commercially available library (New England BioLabs, Beverly, Mass., USA, catalog no. E8110S,Ph.D.-12 phage display peptide library kit). Ten microlitres of M13 phage library (containing about 1011 phage and 109 different sequences) are added to 1 ml of the carbon nanotube suspension. The mixture is incubated at room temperature with mild agitation for 60 minutes. Unbound phage are separated from the nanotube sample by high-speed spinning at 16,000 g in an Eppendorf 5415C centrifuge for 10 minutes. This procedure dissects the phage colony into a set each of binding and non-binding sequences. Phage that remained bound to the nanotubes are eluted by incubating with 0.5 ml of 0.25 M glycine-HCl, pH 3.0, for 10-15 minutes at room temperature followed by centrifuging at 16,000 g for 10 minutes, with the clear supernatant containing the eluted phage. The presence and concentration of binding phage in the supernatant are measured by the number of PFU. This number is measured by serial dilution of the phage pool and plating of the diluted pool with bacteria on an agar plate. Active non-lytic phage, such as M13, retard bacterial growth, and, because the presence of one phage in a cell prevents infection by others (superinfection exclusion), the number of infected bacteria gives a count of the number of active phage particles in the diluted pool. The eluted binding sequences are used for subsequent incubation after in vivo amplification. In subsequent steps, the concentration of Tween 20 is increased sequentially, resulting in stepwise enrichment of the phage pool in favour of the tightest binding sequences. When desired, individual clones are characterized by DNA sequencing. Phage particles differ only in the sequence of the displayed peptide. Therefore, when consensus binding sequences are obtained, differences in binding affinity are often due to the displayed peptide alone. Nevertheless, whether the oligopeptide will independently bind remains a question to be established separately. Preliminary experiments with HiPCO nanotubes established that the concentration of eluted phage decreased monotonically with increasing detergent concentration. This indicated an effective selection pressure by the nonionic detergent Tween 20.

### Example 3.1.1.1 Identification of Proteins with Affinity Towards Metal-Oxides

In this example library molecules (M) are proteins linked by bacteria (L) to DNA tags encoding the proteins. The material used is metal oxides and the medium used is 1 mM D-mannose in M63 salts. Metal-Oxide binding molecules is described in Brown S. (1992, PNAS (89) 8651-8655): Iron oxide is purchased as a dark brown aqueous suspension (catalog no. 4200B) from Advanced Magnetics (Cambridge, Mass.). The following metal oxides are purchased from Aldrich: Fe2O3 (catalog no. 20,351-3), Fe3O4 (catalog no. 31,006-9), and Cr203 (catalog no. 20,306-8). Particles of a size suitable for microscopy are prepared by differential sedimentation of metal oxide suspensions in distilled water. All metal oxides are suspended in M63 salts Logarithmic-phase cultures of the bacterial population are established at 370 C in yeast extract/tryptone (YT) broth (18) supplemented with ampicillin at 200 pug/ml. Expression of the recombinant A receptors is induced by adding isopropyl f3-D-thiogalactoside (IPTG) to a final concentration of 2 mM and incubating at 370 C for 15 min. The bacteria are recovered by centrifugation and resuspended with buffer A (1 mM D-mannose in M63 salts). The D-mannose is included to reduce nonspecific adhesion by the bacterial fimbrae (19). The bacterial suspension is mixed with iron oxide at 200 pug/ml (Advanced Magnetics) in buffer A, and the mixture is incubated at room temperature for 10 min. The iron oxide particles with adhering bacteria are recovered by placing the culture tube next to a magnet. After the magnetic separation, the liquid and nonadhering bacteria are removed by aspiration, and the culture tube is removed from the vicinity of the magnet. The iron oxide is resuspended with M63 salts and spread on YT-ampicillin agar. After overnight incubation of the agar plates at 37° C., colonies appearing on the surface are pooled, exponentially growing cultures are established, and the induction and enrichment procedures are repeated. PCR Analysis and DNA Sequencing. Insert regions in plasmid DNA are PCR amplified (20) 15 cycles with Vent polymerase (New England Biolabs) and indicated primers.

Primers used are upstream PCR primer 5′-ACATCGATGTTGGCTTCGGT-3′, downstream PCR primer 5′-ATCTGCGCTAAACGCACATCG-3′ (see FIG. 1), and RRTVK primer 5′-GCGCCGCACTGTTAAGCA-3′. The RRTVK primer is part of the sense-strand sequence of the first oligonucleotide inserted in pSB1972. The insert region from CsCl-ethidium bromide-purified plasmid DNA is sequenced by the dideoxynucleotide chain-termination sequencing method with upstream and downstream PCR primers and Sequenase (United States Biochemical).

### Example 4.1.1.1 Identification of Polypeptides with Affinity Towards Gold

In this example library molecules (M) are polypeptides linked by bacteria (L) to DNA tags encoding the polypeptides. The material used is gold and the medium used is 90% (v/v) Percol in 10 mM potassium phosphate (pH 7.0), 0.1 M KCl, trypsin, Tris-HCl (pH 8.0) and CaCl2 added to final concentrations of 1 mg/ml, 20 mM and 2 mM. Gold binding molecules are identified essentially as described in Brown S. et al. Journal of Molecular Biology 2000, vol 299, no. 3, pp. 725-735): Populations are enriched for those bacteria adhering to metallic gold in a manner similar to that described (Brown, 1997). Cultures of the populations are established in YT broth supplemented with 25 mg/ml chloramphenicol at 30 degrees C. and expression of the 1-receptor induced for 20 minutes with 0.1 mM IPTG. Induced cultures are diluted into 90% (v/v) Percol in 10 mM potassium phosphate (pH 7.0), 0.1 M KCl and HF-ished gold powder (Brown, 1997) is added to 1 mg/ml.

Adhesion is permitted for 20 minutes at room temperature and the gold with adhering bacteria recovered by centrifugation. The supernatant was discarded and the gold resuspended with YT broth and trypsin, Tris-HCl (pH 8.0) and CaCl2 added to final concentrations of 1 mg/ml, 20 mM and 2 mM, respectively. After 30 minutes at 37° C., all bacteria and gold are recovered by centrifugation and resuspended with the above Percol solution. Gold is removed by centrifugation and the supernatant with released bacteria diluted into YT broth supplemented with 25 mg/ml chloramphenicol and grown overnight. The enrichment procedure is repeated the next day.

### Example 5.1.1.1 Identification of Polypeptides with Affinity Towards Zeolite

Library molecules: Polypeptide

### Tag: Bacterium

Medium: M63 salts containing a final concentration of 75% Percoll

### Material: Zeolite

Zeolite binding molecules are identified essentially as described in Nygaard S et al. adv. Mater. 2002, 1853-1856 14(24) Enrichment for zeolite-binding proteins is conducted and monitored as described [S. Brown, Nat. Biotechnol. 1997, 15, 269.]. Zeolites are washed in sterile 10 mM potassium phosphate, pH 7.0, 0.1 M KCl, and neutral pH is verified before exposure to bacteria.

### Example 6.1.1.1 Identification of Polypeptides with Affinity Towards Gold or Chromium

In this example library molecules (M) are polypeptides linked by bacteria (L) to DNA tags encoding the polypeptides. The materials used are gold and chromium, and the medium used is M63 salts containing a final concentration of 75% Percoll. Chromium or Gold binding molecules are identified as described in Brown S (Nature Biotechnology 1997, (15), 269-272) Cultures are established at 30° C. in YT broth supplemented with 25 microgram/ml chloramphenicol and transcription of the hybrid lamb genes induced by the 2 mM IPTG. This procedure results in approximately 10,000 copies of the hybrid protein to be displayed on the surface of the bacterium. The induced cultures are diluted into M63 salts containing a final concentration of 75% Percoll (Pharmacia, Sweden). At this concentration, the bacteria are less dense than the solution. Metal is added at a concentration of 0.5-1 mg/ml and the bacteria allowed to adhere at room temperature. Following this incubation, the suspensions are centrifuged, the supernatants with the nonadhering bacteria discarded, and the metal with any adhering bacteria resuspended in YT broth supplemented with 25 microgram/ml chloramphenicol. The broth suspensions are incubated overnight, during which the bacteria multiplied under conditions that did not induce expression of the hybrid LamB protein. After each cycle of enrichment, aliquots of the saturated cultures are frozen at −80° C. with 15% glycerol and stored for later analysis.

### Example 7.1.1.1 Identification of Titanium-Binding Peptides

In this example library molecules (M) are peptides linked by bacteriophages (L) to DNA tags encoding the peptides. The materials used are gold and chromium, and the medium used is TBST (0.1%). Titanium binding molecules are identified as described in Liu et al. 2010, Journal of Materials Science: Materials in Medicine, Volume 21, Number 4, Pages 1103-1107. Peptide sequences with preferential binding to titanium based implants are identified by screening the Ph.D.-7TM Phage Display Library (New England Biolabs, #E8100SC), consisting of 2.89E+9 different phages with 7-mer amino acid linear peptide inserts, against cp-Ti disks. Cp-Ti circular disks with 10 mm diameter and 2 mm thickness (Baoji Nonferrous Metal Industry Co., China) are sequentially wet-polished with silicon carbide (SiC) paper to a final finish of 1000 grit, ultrasonically cleaned in ddH2O for 15 min and oxidized with 30% HNO3 solution for 30 min. Thereafter, disks are thoroughly degreased, washed and sonicated in a series of solvents (ethanol, acetone and chloroform for 15 min each) and dried with a stream of nitrogen gas (99.9%). All samples are sterilized under ultraviolet light for 30 min and soaked in ddH2O overnight before use [12]. The ddH2O is removed and replaced with blocking buffer (Tris-HCl buffer solution (50 mM Tris-HCl (pH 7.5), 150 mM NaCl) with 0.1 or 0.5% Tween20 (TBST) and bovine serum albumin (BSA)) and incubated at 4 C for 1 h. The blocking buffer is discarded and the disks are washed six times with TBST (0.1%). An aliquot of the original Ph.D.-7TM library containing 10¹¹ pfu (plaque-forming units, input) diluted in 100 ll of TBST (0.1%) is introduced to each substrate and gently rocked for 1 h at room temperature. Nonbinding phages are discarded and the disks are washed ten times with TBST (0.1%) to remove the nonspecifically or weakly bound phages. The phages remaining on the disk surfaces are then eluted with 0.2 M Glycine/HCl (pH 2.2), containing 1 mg/ml BSA for 10 min at room temperature under gentle agitation. The eluted phages are transferred to a new sterile tube and neutralized with 1 M Tris-HCl (pH 9.1). The number of eluted phages (output) is estimated by infecting Escherichia coli (E. coli) strain ER2738. Eluted phages are then re-amplified with early log phase E. coli ER2738. The amplified phages are isolated by PEG/NaCl (20 w/v % polyethylene glycol-8000 in 2.5 M NaCl) precipitation and used for further biopanning rounds according to the manufacturer's protocols. The similar panning procedures are repeated three more times. The third and fourth panning are different from the earlier two as 0.5% Tween20 is used. Tittering is carried out for both the eluted phages and amplified phages after each round of panning to determine the number of phages. The phages obtained from the third and fourth round are plated on LB agar media containing 5-bromo-4-chloro-3-indolyl-D-galactopyranoside (X-gal) and isopropyl-D-thiogalactopyranosid (IPTG) in appropriate serial dilutions to identify correct plaques. Because the ER2738 strain lacks the lacZ a gene, only the cells infected by M13mp 19 bacteriophage, which carries the lacZ a gene, can produce competent-galactosidase. Therefore, infected cells can hydrolyze. After growing the phages on LB-agar plates, individual blue plaques containing monoclonal phage particles are picked from plates with less than *100 plaques and amplified by infecting 1 ml early-log culture. The culture is centrifuged and 800 ll of the supernatant is precipitated with PEG/NaCl solution. Single stranded DNA is isolated by iodide buffer (10 mM Tris-HCl (pH 8.0), 1 mM EDTA, 4 M NaI), cleaned in 70% ethanol, and finally resuspended in DNase/RNase free water. The purified DNA samples are sequenced by Invitrogen Biotech. Co. Ltd (Shanghai, China) with the NEB-96 gIII sequencing primer (50-HOCCC TCA TAG TTA GCG TAA CG-30) provided in the kit. The amino acid sequence alignments are performed with Align-Version 1.02 (Scientific & Educational Software).

### Example 8.1.1.1 Identification of Polypeptides Binding to Copper Oxide and Zinc Oxide

In this example library molecules (M) are polypeptides linked by bacteria (L) to DNA tags encoding the polypeptides. The material used is Cu2O and ZnO. Binding molecules are identified essentially as described in Thai et al. Biotechnol Bioeng. 2004; 87:129-37. Copper oxide substrates are synthesized electrochemically as previously described (Dai et al., 2004). Briefly, a copper foil working electrode (99.99% purity; Alfa Aesar) is immersed in 1M KOH. Cu2O and Cu(OH)2 films are anodically grown on the foil by control of the sweep potential range during cyclic voltammetry. Cu2O films are formed by sweeping the potential from 1000 to 450 mV, whereas Cu(OH)2 films are formed in the 400- to 125-mV potential range (against SCE). For ZnO substrates, a gold-coated glass working electrode is immersed in 0.1 M Zn(NO3)2 with air bubbling for 2 h and a piece of Zn sheet is used as the counter-electrode. ZnO is cathodically deposited for 30 min on the gold surface at 800 mV and 60jC. All deposited films are thoroughly rinsed with deionized water and air dried. One vial of the FliTrx library (Invitrogen, Carlsbad, Calif.) is used to inoculate 50 mL of IMC supplemented with 50 Ag/mL carbenicillin in a sterile 250-mL shake flask. Cells are grown for 15 to 18 h at 25jC with shaking (250 rpm) to saturation (A600 c 3). Aliquots corresponding to 1010 cells (assuming 109 cells per milliliter per absorbance unit at 600 nm) are transferred to 50 mL of IMC medium supplemented with carbenicillin and 100 Ag/mL L-tryptophan to induce expression of the modified flagellar protein. Cells are grown for 5 or 6 h as just described. Cu2O and ZnO surfaces (10 10 mm) are positioned in the center of 4-cm-diameter polystyrene culture dishes (Corning), washed with 10 mL of sterile deionized water for 2 to 3 min, and incubated with gentle shaking with 10 mL of freshly prepared blocking solution (100 mg dry milk, 300 AL 5 M NaCl, 500 AL 20% methyl a-D-mannopyranoside, 9.2 mL IMC, and 10 AL of 50 mg/mL carbenicillin) for 5 min at 25jC. After decanting the liquid, 10 mL of induced cultures supplemented with 100 mg dry milk, 300 AL of 5 M sterile NaCl, and 500 AL of sterile 20% methyl a-D-mannopyranoside are added to the dish. Cells are allowed to contact the inorganic surface at 25jC for 1 min with agitation at 50 rpm and without agitation for 45 min. At the end of the incubation period, the surface is transferred to a new culture dish containing 10 mL of freshly prepared wash solution (2.5 mL 20% methyl a-D-mannopyranoside, 47.5 mL IMC, 50 AL of 50 mg/mL carbenicillin) using sterile tweezers. The dish is agitated at room temperature and 50 rpm for 5 min. The surface is transferred to a fresh dish as described earlier and the wash operation is repeated a total of five times. To recover binders, the surface is moved to a sterile culture dish containing 10 mL of IMC supplemented with carbenicillin and the plate is vortexed for 30 s to shear off the flagella. The solution is recovered by pipetting, transferred to a sterile 125-mL shake flask, and cells are allowed to grow for 15 to 18 h at 25jC. This completed a round of panning. The entire panning process is repeated five times to enrich for tight binders. Dilutions from overnight cultures obtained after the fifth panning round are plated onto RMG plates (M9 medium, 2% casamino acids, 0.5% glucose, 1 mM MgCl2, 1.5% agar) containing carbenicillin and incubated overnight at 30jC. Randomly selected colonies are used to inoculate 5 mL of RM medium supplemented with carbenicillin and cultures are grown for 16 to 24 h at 30jC. Plasmid DNA is recovered using the QIAprep Spin Miniprep Kit (Qiagen) and the nucleotide sequences of the inserts are determined using the Perkin-Elmer Big Dye PCR sequencing kit and either 5V-ATTCACCTGACTGACGAC-3V as a forward primer or 5V-CCCTGATATTCGTCAGCG-3V as a reverse primer.

### Example 9.1.1.1 Indentification of Peptides Binding to Conductive Polymers

In this example library molecules (M) are peptides linked by bacteriophages (L) to (genomic) DNA tags encoding the peptides. The material used is a conducting polymer. Conducting polymer binding molecules are identified essentially as described in Sanghvi et al. Nature Materials 4, 496-502 (2005). To select peptides for PPyCl using phage display, we synthesized thin films (200-250 nm) of PPyCl by standard electrochemical methods. PPyCl thin films are incubated with a commercially available 12-mer linear phage display library (M13 clone, filamentous bacteriophage), which expressed random peptides on minor coat proteins (pIII) with a diversity of 2.7×109 (Ph.D.-12 Phage Display Peptide Library Kit, New England Biolabs). The sequences, which correspond to the peptides displayed on the phage, are analysed using DNA sequencing

### Example 10.1.1.1 Identification of Peptides Binding to a Semiconductive Material

In this example library molecules (M) are peptides linked by bacteriophages (L) to (genomic) DNA tags encoding the peptides. The material used is a semiconductor, and the medium is Tris-buffered saline (TBS) containing 0.1% TWEEN-20. Semiconductor binding molecules are identified essentially as described in Whaley et al. Nature 405, 665-668 Different single-crystal semiconductors: GaAs(100), GaAs(111)A, GaAs(111)B, InP(100) and Si(100). These substrates allowed for systematic evaluation of the peptide-substrate interactions. Protein sequences that successfully bound to the specific crystal are eluted from the surface, amplified by 106, and re-reacted against the substrate under more stringent conditions. This procedure is repeated five times to select the phage with the most specific binding. After the third, fourth and fifth rounds of phage selection, crystal-specific phage are isolated and their DNA sequenced. We identified peptide binding that is selective for the crystal composition (for example, binding to GaAs but not to Si) and crystalline face (for example, binding to (100) GaAs, but not to (111)B GaAs). The library is exposed to the semiconductor crystals in Tris-buffered saline (TBS) containing 0.1% TWEEN-20, to reduce phage±phage interactions on the surface. After rocking for 1 h at room temperature, the surfaces are washed with 10 exposures to Tris-buffered saline, pH 7.5, and increasing TWEEN-20 concentrations from 0.1% to 0.5% (v/v). The phage are eluted from the surface by the addition of glycine±HCl (pH 2.2) for 10 min, transferred to a fresh tube and then neutralized with Tris±HCl (pH 9.1). The eluted phage are titred and binding efficiency is compared. The phage eluted after third-round substrate exposure are mixed with their Escherichia coli ER2537 host and plated on LB XGal/IPTG plates. Since the library phage are derived from the vector M13mp19, which carries the lacZa gene, phage plaques are blue in colour when plated on media containing Xgal (5-bromo-4-chloro-3-indoyl-b-Dgalactoside) and IPTG (isopropyl-b-D-thiogalactoside). Blue/white screening is used to select phage plaques with the random peptide insert. Plaques are picked and DNA sequenced from these plates.

### Example 11.1.1.1 Identification of Peptides Binding to Graphene

In this example library molecules (M) are peptides linked by bacteriophages (L) to (genomic) DNA tags encoding the peptides. The material used is graphene, and the medium is Tris buffered saline containing 0.1-0.8% Tween-20 (TBST). Graphene binding molecules are identified essentially as described in Cui et al. Nano Lett. 2010, 10, 4559-4565 We used three different forms of commercial graphene/graphite samples as targets for panning against peptide libraries: TIMREX SLP30 Primary Synthetic Potato graphite (TIMCAL, Westlake, Ohio, surface area 8.0 m2/g); N006 nano graphene platelets (Angstron Materials, Dayton, Ohio, surface area up to ˜2675 m2/g); and AFM standard highly ordered pyrolytic graphite (HOPG, SPI, West Chester, Pa., surface area not available). Briefly, 7-mer or 12-mer peptide phage libraries are incubated with SLP30, N006, or HOPG graphene/graphite flakes in Trisbuffered saline containing 0.1-0.8% Tween-20 (TBST) for 1 h at room temperature. The graphite particles are then washed several times with TBST buffer. The phages are eluted from the particles by addition of glycine-HCl (pH 2.2) for 5 min, neutralized with Tris-HCl, pH 9.1, amplified, and subjected to additional pannings. Eluted phages are then amplified in E. coli, and the process repeated for up to five rounds of biopanning, under increasingly stringent conditions, to obtain phage clones expressing peptides having the highest binding affinities to the graphene/graphite samples. After the final round of panning, DNA sequence analysis of the isolated phage clones yielded heptameric or dodecameric graphene-binding peptides (GBPs).

In the following examples, the source of ligands are varied and the screening is done as described in the indicated general example.

| | Based on general | Library | | |
|---|---|---|---|---|
| Example | example | molecules (M) | Tags (T) | Outcome |
| 4.2.1.1 | 4.1.1.1 | Macrocyclic | DNA | Macrocyclic |
| | | molecules | | molecules with |
| | | | | affinity towards gold |
| 5.2.1.1 | 5.1.1.1 | Peptides | DNA | Peptides with |
| | | | | affinity towards |
| | | | | zeolite |
| 6.2.1.1 | 6.1.1.1 | Small organic | PNA | Small organic |
| | | molecules | | molecules with |
| | | | | affinity towards |
| | | | | gold or chromium |
| 7.2.1.1 | 7.1.1.1 | Macrocyclic | PNA | Macrocycles with |
| | | molecules | | affinity |
| | | | | towards titanium |
| 8.2.1.1 | 8.1.1.1 | peptides | PNA | Peptides with |
| | | | | affinity towards |
| | | | | copper oxide and |
| | | | | zinc oxide |
| 9.2.1.1 | 9.1.1.1. | Small organic | DNA | Small organic |
| | | molecules | | molecules with |
| | | | | affinity towards |
| | | | | conductive polymers |

### Example 11.2.1.1 Identification of Small Organic Molecules Binding to Graphene

Binding molecules are identified essentially as described in Example 11.1.1.1 Except the following molecules are used: Small molecules linked to (encoded by) polynucleotide tags. Except that library molecules (M) are small organic molecules linked by a PEG6 linker (6 repeated polyethylene glycol units) to DNA tags encoding the molecules. The material used is graphene, and the medium is Tris buffered saline containing 0.1-0.8% Tween-20 (TBST). Graphene binding molecules are identified essentially as described in Cui et al. Nano Lett. 2010, 10, 4559-4565 We used three different forms of commercial graphene/graphite samples as targets for panning: TIMREX SLP30 Primary Synthetic Potato graphite (TIMCAL, Westlake, Ohio, surface area 8.0 m2/g); N006 nano graphene platelets (Angstron Materials, Dayton, Ohio, surface area up to ˜2675 m2/g); and AFM standard highly ordered pyrolytic graphite (HOPG, SPI, West Chester, Pa., surface area not available). Briefly, a library of polynucleotide-tagged small molecules are incubated with SLP30, N006, or HOPG graphene/graphite flakes in Trisbuffered saline containing 0.1-0.8% Tween-20 (TBST) for 1 h at room temperature. The graphite particles are then washed several times with TBST buffer. The polynucleotide-tagged small molecules are eluted from the particles by addition of glycine-HCl (pH 2.2) for 5 min, neutralized with Tris-HCl, pH 9.1, amplified, and subjected to additional pannings. Eluted polynucleotide-tagged small molecules are then amplified, and the process repeated for several rounds, under increasingly stringent conditions, to obtain polynucleotide-tagged small molecules having the highest binding affinities to the graphene/graphite samples. After the final round of panning, the polynucleotide sequences of the polynucleotide-tagged small molecules are determined by sequencing.

### Example 12.1.1.1 Identification of Fragments Binding Via —S—S— Bonds to a Material with Exposed —SH Groups

In this example library molecules (M) are fragments with disulphide (—S—S—) groups without tags but screened in pools containing fragments of different sizes such that a fragment can be identified via its mass. The material is a thiol-functionalized material (a protein in this case). The material used is graphene, and the medium is buffer containing 25 mM potassium phosphate (pH 7.5) and 1 mM 2-mercaptoethanol. Other similar buffers and reducing agents may also be used. Thiol-functionalized-Protein-binding molecules are identified as described in Erlanson et al. PNAS; Aug. 15, 2000; vol. 97; no. 17; 9367-9372 In a typical experiment, 1 ml of a DMSO solution containing a library of 8-15 disulfide containing compounds is added to 49 ml of protein-containing buffer. These compounds are chosen so that each has a unique molecular weight; ideally, these molecular weights differ by at least 10 atomic mass units so that deconvolution is unambiguous. Although we have typically chosen to screen pools of 8-15 disulfide-containing compounds for ease of deconvolution, larger pools can be used as discussed below and as shown in FIG. 2C. The protein is present at a concentration of 15 mM, each of the disulfide library members is present at 0.2 mM, and thus the total concentration of all disulfide library members is 2 mM. The reaction is done in a buffer containing 25 mM potassium phosphate (pH 7.5) and 1 mM 2-mercaptoethanol, although other buffers and reducing agents can be used. The reactions are allowed to equilibrate at ambient temperature for at least 30 min. These conditions can be varied considerably depending on the ease with which the protein ionizes in the mass spectrometer, the reactivity of the specific cysteine(s), etc. In the case of TS, the conditions described above are found to be satisfactory. No special effort is made to exclude oxygen or adventitious metal ions; on the time scale of these reactions, there is sufficient free thiol to facilitate disulfide exchange. After equilibration, the reaction is injected onto an HP1100 HPLC and chromatographed on a C18 column attached to a mass spectrometer (Finnigan-MAT LCQ, San Jose, Calif.). The multiply charged ions arising from the protein are deconvoluted with available software (XCALIBUR) to arrive at the mass of the protein. The identity of any library member bonded through a disulfide bond to the protein is then easily determined by subtracting the known mass of the unmodified protein from the observed mass. This process assumes that the attachment of a library member does not dramatically change the ionization characteristics of the protein itself, a conservative assumption because in most cases the protein will be at least 20-fold larger than any given library member. This assumption is confirmed by demonstrating that small molecules selected by one protein are not selected by other proteins (data not shown).

Tethering Scheme:

Principles of tethering, where monophore:target interaction is weak (a) thiolate disulfide exchange disfavors adduct formation. Longer-lived association (b) yields more product. Binding of fragments can be accompanied by conformational changes (c) in both ligand and target (allostery). Extended tethering employs an irreversible alklyator plus a thiol, enabling further fragments (orange) to be found at more distant binding sites (d). (e) Typical tethering monophores design.

### Example 13.1.1.1 Identification of Small Organic Molecules Binding to a Material Using Affinity Screening and Decoding by Mass Spectroscopy

In this example library molecules (M) are fragments without tags. The molecules are screened in pools containing fragments of different sizes such that a fragment can be identified in the screening output on the basis of its mass. The material is a protein and the medium is pH 7.5 phosphate buffer containing 2.5% DMSO and 100 mM NaCl. Material-binding molecules are identified essentially as described in Annis et al. 2004; International Journal of Mass Spectrometry 238 (2004) 77-83 AS-MS analysis are conducted by incubating 2500-member libraries at 2.5 mM cumulative compound concentration with 5 M E. coli DHFR [38] in a final volume of 2 L pH 7.5 phosphate buffer containing 2.5% DMSO and 100 mM NaCl. As such, 2 pmol of each library component (at 1.0 M/component) and 10 pmol (0.18 g) protein are used in a single analysis. The use of excess protein relative to each library member minimizes competition between multiple binders in a given library. Typical sample preparation protocol is as follows: to 1 L of a DMSO solution of 100 mM 2500-member library is added 19 L pre-warmed (37° C.) pH 7.5, 50 mM phosphate buffer containing 100 mM NaCl and 0.1 mM dithioethrythritol. The resulting solution is mixed by repeated pipetting and centrifuged at 10,000×g for 10 min. A 1.0 L aliquot of the supernatant is added to 1.0 L of a 10 M solution of purified DHFR in pH 7.550 mM phosphate buffer containing 100 mM NaCl and 0.1 mM dithioethrythritol. Samples are incubated at room temperature for 30 min and then chilled at 4° C. pending AS-MSanalysis. Discrete compound screening and competition experiments are prepared identically except that compound stock concentrations in DMSO are adjusted such that the final DMSO concentration in each protein-containing sample is 2.5%.

Further examples using the general examples described above:

| | Based on general | Library | | |
|---|---|---|---|---|
| Example | example | molecules (M) | Tags (T) | Outcome |
| 1.2.1.1 | 1.1.1.1 | Small organic | DNA | Small organic |
| | | molecules | | molecules with |
| | | | | affinity for CNTs |
| 2.2.1.1. | 2.1.1.1 | Small organic | DNA | Small organic |
| | | molecules | | molecules with |
| | | | | affinity for MWCNTs |
| 3.2.1.1. | 3.1.1.1 | Small organic | DNA | Small organic |
| | | molecules | | molecules with |
| | | | | affinity for metal |
| | | | | oxides |
| 12.2.1.1 | 12.1.1.1 but where the | Small organic | No tags, molecules | Small organic |
| | material is thiol- | molecules | are identified by | molecules with |
| | functionalized CNT | | mass spectrometry | affinity for CNT |
| 13.2.1.1 | 13.1.1.1 | Small organic | DNA | |
| | | molecules | | |

Further examples using the general examples described above:

| | Based on general | Library | | |
|---|---|---|---|---|
| Example | example | molecules (M) | Material | Outcome |
| 1.3.1.1 | 1.1.1.1 | Fragments with | Thiol- | Small organic |
| | | disulphides | functionalized | molecules with |
| | | | carbon | affinity for |
| | | | nanotubes | CNT |
| 11.3.1.1 | 11.1.1.1 | Fragments with | Thiol- | Small organic |
| | | disulphides | functionalized | molecules with |
| | | | graphene | affinity for |
| | | | | graphene. |

Further examples using the general examples described above:

| | Based on general | |
|---|---|---|
| Example | example | Medium used |
| 1.1.2.1 | 1.1.1.1 | Acetonitrile (>99.9% MeCN, <0.1% H2O) comprising 0.1%-10% |
| | | epoxy repeat units (a mixture comprising |
| | | 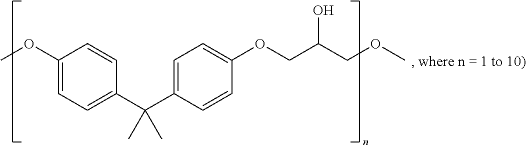 |
| 2.1.2.1 | 2.1.1.1 | Dichloromethane (>99.9% DCM, <0.1% H2O) comprising 0.1%- |
| | | 10% nylon repeat units (a mixture comprising |
| | | 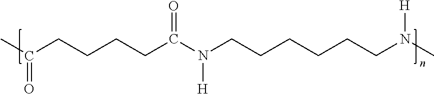 |
| | | Where n = 1 to 10. |
| 3.1.2.1 | 3.1.1.1 | Tetrahydrofurane (>99.9% THF, <0.1% H2O) comprising 0.1%- |
| | | 10% polypropylene repeat units (a mixture comprising |
| | | 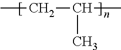 |
| | | Where n = 1 to 10 |
| 4.1.2.1 | 4.1.1.1 | Ethanol (1-10%) comprising 0.1%-10% repeat units (a mixture |
| | | comprising X—[CH2—CH2]n—Y, where n = 1 to 10; and X and Y is |
| | | individually —OH, —CH3, —COOH, —NH2, —CHO, or —H]) |
| 5.1.2.1 | 5.1.1.1 | Ethanol (>5%) comprising 0.1%-10% repeat units (a mixture |
| | | comprising X—[CH2—CH2]n—Y, where n = 1 to 10; and X and Y is |
| | | individually —OH, —CH3, —COOH, —NH2, —CHO, or —H]) |
| 6.1.2.1 | 6.1.1.1 | Ethanol (>50%) comprising 0.1%-10% repeat units (a mixture |
| | | comprising X—[CH2—CH2]n—Y, where n = 1 to 10; and X and Y is |
| | | individually —OH, —CH3, —COOH, —NH2, —CHO, or —H]) |
| 10.1.2.1 | 10.1.1.1 | Acetonitrile (>1%) comprising 0.1%-10% repeat units (a mixture |
| | | comprising X—[CH2—CH2]n—Y, where n = 1 to 10; and X and Y is |
| | | individually —OH, —CH3, —COOH, —NH2, —CHO, or —H]) |

### Example 11.1.2.1 Identification of Small Organic Molecules Binding to Carbon Nanotubes

Binding molecules are identified essentially as described in Example 11.2.1.1, except the following material is used: Carbon nanotubes and the following medium is used: 10 mM potassium phosphate, pH 7.0, 0.1 M KCl, 1% Triton X-100 comprising 0.1-10% Acetonitrile and comprising 0.1%-10% repeat units (a mixture comprising X—[CH2-CH2]n-Y, where n=1 to 10; and X and Y is individually —OH, —CH3, —COOH, —NH2, —CHO, or —H]). Carbon nanotube binding molecules are identified essentially as described in Cui et al. Nano Lett. 2010, 10, 4559-4565 We used three different forms of commercial graphene/graphite samples as targets for panning: TIMREX SLP30 Primary Synthetic Potato graphite (TIMCAL, Westlake, Ohio, surface area 8.0 m2/g); N006 nano graphene platelets (Angstron Materials, Dayton, Ohio, surface area up to ˜2675 m2/g); and AFM standard highly ordered pyrolytic graphite (HOPG, SPI, West Chester, Pa., surface area not available). Briefly, a library of polynucleotide-tagged small molecules are incubated with SLP30, N006, or HOPG graphene/graphite flakes in Trisbuffered saline containing 0.1-0.8% Tween-20 (TBST) for 1 h at room temperature. The graphite particles are then washed several times with TBST buffer. The polynucleotide-tagged small molecules are eluted from the particles by addition of glycine-HCl (pH 2.2) for 5 min, neutralized with Tris-HCl, pH 9.1, amplified, and subjected to additional pannings. Eluted polynucleotide-tagged small molecules are then amplified, and the process repeated for several rounds, under increasingly stringent conditions, to obtain polynucleotide-tagged small molecules having the highest binding affinities to the graphene/graphite samples. After the final round of panning, the polynucleotide sequences of the polynucleotide-tagged small molecules are determined by sequencing.

### Example 12.1.2.1 Identification of Fragments in the Presence of a Soluble Polymer During Screening

Binding molecules are identified essentially as described in Example 12.1.1.1

Except the following medium is used: Acetonitrile (0.1-1%) comprising 0.1%-10% repeat units (a mixture comprising X—[CH2-CH2]n-Y, where n=1 to 10; and X and Y is individually —OH, —CH3, —COOH, —NH2, —CHO, or —H])

### Example 13.1.2.1 Identification of Small Organic Molecules in the Presence of a Soluble Polymer During Screening

Binding molecules are identified essentially as described in Example 13.1.1.1

Except the following medium is used: Acetonitrile (1-10%) comprising 0.1%-10% repeat units (a mixture comprising X—[CH2-CH2]n-Y, where n=1 to 10; and X and Y is individually —OH, —CH3, —COOH, —NH2, —CHO, or —H])

Further examples using the general examples described above, but where other materials are used, are listed in the following table:

| | Based on | |
|---|---|---|
| | general | |
| Example | example | Material used instead of that used in the general example |
| 1.1.1.2 | 1.1.1.1 | Multi-walled carbon nanotubes (MWCNT, Cheap Tubes, Inc., USA) |
| 2.1.1.2 | 2.1.1.1 | Graphene PureSheets MONO NanoIntegris Technologies, Inc., Canada |
| 3.1.1.2 | 3.1.1.1 | Graphane, made from graphene using the method of Ph 2012, |
| | | Nanoscale. 2012 Nov. 21; 4(22): 7006-11 |
| 4.1.1.2 | 4.1.1.1 | Fullerenes (mixture of buckyballs [Fullerene-C60, Sigma |
| | | Aldrich, Cat. No. 379646], graphene, carbon nanotubes) |
| 5.1.1.2 | 5.1.1.1 | Hydroxy-Functionalized fullerenes (mixture of buckyballs, graphene, |
| | | carbon nanotubes hydroxylated as described in Li et al., 1993, |
| | | Journal of the Chemical Society, Chemical Communications (23): 1784.) |
| 6.1.1.2 | 6.1.1.1 | Defect fullerenes, i.e. graphene and carbon nanotubes where the defect |
| | | comprise sp3-hybridized carbon atoms introduced by laser irradiation. |
| 7.1.1.2 | 7.1.1.1 | Carbon nanotubes (HiPco SuperPurified, NanoIntegris Technologies, |
| | | Inc., Canada |
| 8.1.1.2 | 8.1.1.1 | Carbon nanotubes (HiPco SuperPurified, NanoIntegris Technologies, |
| | | Inc., Canada |
| 9.1.1.2 | 9.1.1.1 | Ceramic, Silicon carbide nanopowder, <100 nm particle size from Sigma |
| | | (product no. 594911) |

### Example 14.1.1.1 Particle Size Fractionation of Glutathione Monolayer-Protected Gold Nanoparticles

The purpose of this Example is to demonstrate the size fractionation of glutathione (GSH) monolayer protected gold nanoparticles from an aqueous solution. The method comprises the fractional precipitation of the stabilized, charged, water-soluble nanoparticles by addition of a substantially water-miscible organic solvent in the presence of an electrolyte. Preparation of Glutathione Monolayer-Protected Gold Nanoparticles. Unless otherwise noted, all reagents are purchased from Aldrich (Milwaukee, Wis.) and are used without further purification. In a typical reaction, 60 mL of methanol (HPLC grade) and 10 mL of acetic acid (HPLC grade) are mixed in an Erlenmeyer flask by stirring for 2-5 min. Tetrachloroauric acid (HAuC14.xH20, 99.99%) (0.37 g) and 61.4 mg of glutathione (GSH) (99% minimum, obtained from Sigma, St. Louis, Mo.) are added to the above mixed solvents and dissolved by stirring for 5 min, resulting in a clear, yellow solution. A sodium borohydride solution is prepared by dissolving 0.6 g of NaBH4 (99%) in 30 g of Nanopure® water. The NaBH4 solution is added dropwise into the above solution with rapid stirring. When the first drop of NaBH4 solution is added, the HAuC14 solution immediately turned dark brown from yellow. This reaction is exothermic, warming the solution for approximately 15 min. During the reaction, the pH of the solution changed from 1.2 to about 5.0. The reaction solution is stirred rapidly for 2 h. The glutathione monolayer-protected gold nanoparticles are soluble in water and when diluted, the solution became clear purple. This preparation method results in nanoparticles with a broad size distribution. Fractionation of Glutathione Monolayer-Protected Gold Nanoparticles. The GSH monolayer-protected gold nanoparticles (0.3 g) are dissolved in 50 mL of a 100 mM sodium chloride solution. The first fraction of the nanoparticles is precipitated out by adding methanol to the nanoparticle solution to a final content of 14% by volume. The nanoparticles are collected by centrifugation at 4000 rpm for 1 min in a Sorvall® RT7 PLUS centrifuge (Kendra Laboratory Products, Newtown, Conn.). Then, more methanol is added to the supernatant to a final content of 18% by volume and the precipitated nanoparticles are collected as described above as the second fraction. This step-wise addition of methanol is continued and nanoparticle fractions 3-7 are collected at methanol concentrations of 22%, 26%, 30%, 34% and 38% by volume, respectively.

### Example 14.2.1.1 Identification of Small Molecule Ligands with Affinity to Carbon Nanotubes

Binding molecules are identified essentially as described in Example 14.1.1.1, except carbon nanotubes are used as material instead of gold and a library of organic molecules linked to (encoded by) DNA is used to coat the carbon nanotubes. Coated carbon nanotubes are collected and the identity of the organic molecules which coat the nanotubes are identified by sequencing the polynucleotide tag.

### Example 15.0.0.0 Identification of Ligands to Carbon Nanotubes in the Presence of PPyCl

Binding molecules, which bind efficiently to a filler (carbon nanotubes) but not to a polymer matrix (PPyCl) of a composite material, are identified essentially as described in example 1.1.1.1. The library of molecules comprises the following molecules; polynucleotide-tagged THRTSTLDYFVI-pyrene (which can bind to both carbon nanotubes and PPyCl) and polynucleotide-tagged pyrene (which binds efficiently to carbon nanotubes but not PPyCl). In a first experiment, binding molecules are identified essentially as described in example 1.1.1.1. Thus both THRTSTLDYFVI-pyrene and polynucleotide-tagged pyrene are identified as carbon nanotube binding molecules. In a second experiment, binding molecules are identified essentially as described in example 1.1.1.1 but the medium comprises repeat units of PPyCl. During an incubation step, polynucleotide-tagged THRTSTLDYFVI-pyrene molecules binds to the PPyCl repeat units and are removed during the partitioning step, whereas polynucleotide-tagged pyrene molecules are retained during the partitioning step. Such pyrene molecules without the THRTSTLDYFVI moiety bind efficiently to a filler (carbon nanotubes) but not to a polymer matrix (PPyCl) of a composite material.

### Example T.1.1.1.1.1.1.1.1

CNT-binding peptide are enriched from a mixture of CNT-binding peptides and control peptides all bound to bacteriophages using the following method. In this example, the library molecules were peptides fused to M13 bacteriophage tags. The medium used was TNT buffer (10 mM Tris-HCl, pH 8; 10 mM NaCl; 1% Triton X-100) and the material used is carbon nanotubes (HiPco SuperPurified, NanoIntegris Technologies, Inc., Canada), respectively.

### Step 1:

Carbon nanotubes (CNTs) were dispersed in TNT buffer by sonication using 20% power for 4 minutes in a Qsonica Q700 sonicator with a cup horn installed. The CNT concentration is 0.02 mg/ml. CNTs were washed twice in TNT buffer and collected by centrifugation at 20,000 g for 5 minutes.

### Step 2.

Bacteriophages displaying peptide A2 (IFRLSWGTYFS, inserted in the phagemid pCANTAB using restriction sites NcoI and NotI) and wildtype pIll in the form of mini-M13 bacteriophages (Brown et al. 2015: Brown, S., Majikes, J., Martinez, A., Giron, T. M., Fennell, H., Samano, E. C., LaBean, T. H. 2015. An easy-to-prepare mini-scaffold for DNA origami. Nanoscale 7, 16621-16624) were mixed with the CNTs and allowed to bind to the CNTs for 1 hour at room temperature. The ratio between bacteriophages displaying a CNT-binding peptide (A2) and bacteriophages displaying a non-CNT-binding peptide (mini-M13) is 1:5 and the total number of bacteriophages added is 1.1E+9.

### Step 3:

CNTs were washed with TNT-buffer 1 time, 3 times or 10 times and collected by centrifugation. After the last wash, the CNTs were suspended in TNT-buffer and transferred to an Escherichia coli strain TG1 culture and the bacteriophages were allowed to infect Escherichia coli.

### Step 4.

For easy evaluation of enrichment by blue/white screening, the mini-M13 bacteriophages prepared by Brown et al. 2015 using phagemid pSB4423 were prepared using pBluescriptKS(−) instead, such that the mini-M13 bacteriophages displaying the non-CNT-binding peptide carried the lacZα gene whereas the bacteriophages displaying the CNT-binding peptide did not. After infection (step 3), the bacterial cells were plated on LB (lysogenic broth) agar plates containing 100 μg/ml amp (ampicillin), 40 μg/ml X-gal (5-bromo-4-chloro-3-indolyl-β-D-galactopyranoside) and 0.1 mM IPTG (isopropyl 3-D-1-thiogalactopyranoside, and incubated 16 hours at 37° C.

### Step 5:

Enrichment for the CNT-binding peptide A2 is evaluated based on the changes in the ratio between bacteriophages displaying peptide A2 and bacteriophages displaying wildtype pIII (mini-M13), and is measured by counting blue and white colonies on LB agar plates containing 100 μg/ml amp, 40 μg/ml X-gal and 0.1 mM IPTG. The input and output ratios as well as the enrichment folds obtained are shown in the table below.

| | Input ratio | Output ratio | |
|---|---|---|---|
| No. of washes | (A2:mini-M13) | (A2:mini-M13) | Enrichment fold |
| 1 | 1:5 | 20:1 | 100 |
| 3 | 1:5 | 17:1 | 85 |
| 10 | 1:5 | 18.2:1 | 91 |

### Varying the CNT Concentration

In the following variant examples, enrichment for the CNT-binding peptide A2 is carried out essentially as described in Example T.1.1.1.1.1.1.1.1, except the CNT concentration used was varied. Enrichment for the CNT-binding peptide A2 was evaluated based on the changes in the ratio between bacteriophages displaying peptide A2 and bacteriophages displaying wildtype pIII (mini-M13), and was measured by counting blue and white colonies on LB agar plates containing 100 μg/ml amp, 40 g/ml X-gal and 0.1 mM IPTG as described earlier (cf. example T.1.1.1.1.1.1.1.1). The outcome obtained is listed in the table below.

| | Bacteriophages | CNT | | | |
|---|---|---|---|---|---|
| Example | added | concentration | | Enrichment fold | |
| T.1.1.1.1.1.2.1.1 | 1.1E+9 | 0.2 | mg/ml | 23 | (one wash) |
| | | | | 16 | (three washes) |
| | | | | 17 | (ten washes). |
| T.1.1.1.1.1.3.1.1 | 1.1E+9 | 2 | mg/ml | 4 | (one wash) |
| | | | | 2 | (three washes) |
| | | | | 2 | (ten washes). |

### Varying the Total Number of Bacteriophages Added

In the following variant examples, enrichment for the CNT-binding peptide A2 was carried out essentially as described in Example T.1.1.1.1.1.1.1.1, T.1.1.1.1.1.2.1.1 and T.1.1.1.1.1.3.1.1, except the total number of bacteriophages added was varied. Enrichment for the CNT-binding peptide A2 was evaluated based on the changes in the ratio between bacteriophages displaying peptide A2 and bacteriophages displaying wildtype pIII (mini-M13), and was measured by counting blue and white colonies on LB agar plates containing 100 μg/ml amp, 40 μg/ml X-gal and 0.1 mM IPTG as described earlier (cf. example T.1.1.1.1.1.1.1.1). The outcome obtained is listed in the table below.

| | Bacteriophages | CNT | | | |
|---|---|---|---|---|---|
| Example | added | concentration | | Enrichment fold | |
| T.1.1.1.1.2.1.1.1 | 1.1E+11 | 0.02 | mg/ml | 2 | (one wash) |
| | | | | 16 | (three washes) |
| | | | | 8 | (ten washes) |
| T.1.1.1.1.2.2.1.1 | 1.1E+11 | 0.2 | mg/ml | 7 | (one wash) |
| | | | | 16 | (three washes) |
| | | | | 19 | (ten washes) |
| T.1.1.1.1.2.3.1.1 | 1.1E+11 | 2 | mg/ml | 10 | (one wash) |
| | | | | 4 | (three washes) |
| | | | | 3 | (ten washes) |

### Example T.1.1.1.1.2.2.2.1

Binding molecules were identified essentially as described in Example T.1.1.1.1.2.2.1.1, except 1 min. sonication at 35 kHz and 160 W (Sonorex Digitec DT 31 H, Bandelin, Germany) was applied during washing steps to increase the washing efficiency. In this example, the obtained enrichment folds were 17 (three washes) and 15 (ten washes).

### Varying the Source of Ligands

### Example T.1.2.1.1.2.1.1.2

Binding molecules are identified essentially as described in Example T.1.1.1.1.2.1.1.1, except the bacteriophages display a peptide library with a complexity of 4E+10, i.e. 4E+10 different peptides are displayed on the bacteriophages. Each of the displayed peptides is a linear peptide consisting of 12 amino acids. After selection, the bacteriophages are not allowed to infect Escherichia coli. Instead, PCR is performed on the entire selection output and the enrichment of CNT-binding peptide ligands is evaluated using next-generation sequencing, where the frequency of occurrence of specific peptide sequences from the library is counted. In this example, a large number, e.g. 1,000, of linear CNT-binding peptides is identified.

### Example L.1.2.1.1.2.1.1.2

Binding molecules are identified essentially as described in Example T. 1.2.1.1.2.1.1.2, except the library used is prepared as described in Lam et al. 1991 (Nature 354; 82-84). Briefly, a library of about 2.5 million pentapeptides are synthesized on beads in a split-and-mix fashion using 10 reaction vessels to incorporate all natural amino acids except cysteine (to avoid disulphide formation). Following screening on CNT, where the frequency of occurrence of specific peptide sequences from the library is counted. In this example, a large number, e.g. 1,000, of linear CNT-binding peptides is identified.

### Example E.1.2.1.1.2.1.1.2

Binding molecules are identified essentially as described in Example T. 1.2.1.1.2.1.1.2, except the library used is prepared as described in Melkko et al. 2004 (Nat. Biotechnol. 22 (2004), 568-574.).

Briefly, Small organic molecules are coupled to amino modified oligonucleotides, containing a hybridization domain and a unique coding sequence, which ensure the identity of the coupled molecule. DNA strands are then allowed to form duplexes, and the duplexes, wherein each strand is linked to—and encodes—a small molecule, are screened on CNT as shown in the following scheme where the target is CNT:

Following screening on CNT, where the frequency of occurrence of specific peptide sequences from the library is counted. In this example, number, of CNT-binding small molecules is identified.

### Example T.1.3.1.1.2.1.1.2

Binding molecules are identified essentially as described in Example T.1.1.1.1.2.1.1.1, except the bacteriophages display a peptide library with a complexity of 4.5E+9, i.e. 4.5E+9 different peptides are displayed on the bacteriophages. Each of the displayed peptides is a cyclic peptide consisting of 12 amino acids, where amino acids 3 and 10 are cysteines to allow cyclization of the peptide under oxidizing conditions. After selection, the bacteriophages are not allowed to infect Escherichia coli. Instead, PCR is performed on the entire selection output and the enrichment of CNT-binding peptide ligands is evaluated using next-generation sequencing, where the frequency of occurrence of specific peptide sequences from the library is counted. In this example, a large number, e.g. 1,000, of cyclic CNT-binding peptides is identified.

### Example T.2.4.6.1.3.1.1.2

Binding molecules are identified essentially as described in Example T.1.1.1.1.1.1.1.1, except the tag is mRNA instead of bacteriophages and the medium is SA-binding buffer (40 mM Tris(hydroxymethyl)aminomethane, 300 mM KCl, 2 mM EDTA, 0.1% Triton X-100, 5 mM 2-mercaptoethanol, 100 mg/ml BSA, 1 mg/ml tRNA, pH 7.4). A library of 1E+13 random peptide sequences is prepared as described by Wilson et al. 2001 (Wilson, D. S., Keefe, A. D., Szostak, J. W. 2001. The use of mRNA display to select high-affinity protein-binding peptides. Proceedings of the National Academy of Sciences, vol. 98, no. 7). After selection, PCR is performed on the entire selection output and the enrichment of CNT-binding peptide ligands is evaluated using next-generation sequencing, where the frequency of occurrence of specific peptide sequences from the library is counted. In this example, a large number, e.g. 10,000, of CNT-binding peptides is identified.

### Varying the Type of Material Used for Selection

Binding molecules are identified essentially as described in Example T. 1.2.1.1.2.1.1.2, except the material used to search for binding molecules is varied.

| Example | Material | Manufacturer | Outcome |
|---|---|---|---|
| T.1.2.1.2.2.1.1.2 | Metallic single wall carbon | NanoIntegris Technologies, | >100 peptides that can bind |
| | nanotubes (SWNTs) | Inc., Canada | to metallic SWNTs |
| T.1.2.1.3.2.1.1.2 | Semiconducting single wall | NanoIntegris Technologies, | >100 peptides that can bind |
| | carbon nanotubes (SWNTs) | Inc., Canada | to semiconducting SWNTs |
| T.1.2.1.4.2.1.1.2 | Plasma carbon nanotubes | NanoIntegris Technologies, | >100 peptides that can bind |
| | (CNTs) | Inc., Canada | to plasma CNTs |
| T.1.2.1.5.2.1.1.2 | Multiwalled carbon | Cheap Tubes, Inc., USA | >100 peptides that can bind |
| | nanotubes (MWNT) | | to MWNTs |
| T.1.2.1.6.2.1.1.2 | Graphene PureSheets | NanoIntegris Technologies, | >100 peptides that can bind |
| | MONO | Inc., Canada | to graphene |
| T.1.2.1.7.2.1.1.2 | Graphene nanoplatelets | Cheap Tubes, Inc., USA | >100 peptides that can bind |
| | (GNPs) | | to GNPs |

### Varying the Medium Composition Used for Selection

Binding molecules are identified essentially as described in Example T. 1.2.1.1.2.1.1.2, except the medium composition used to search for binding molecules is varied. To find molecules that can bind in e.g. an organic solvent, the medium composition needs to contain said solvent during selection. Examples of organic solvents are, but not limited to, acetonitrile, dimethyl formaide, propylene carbonate and chloroform. Bacteriophages have shown excellent stability organic solvents, e.g. Olofsson et al. 1998 (Olofsson, L., Ankarloo, J., Nicholls, I. A. 1998. Phage viability in organic media: insights into phage stability. Journal of Molecular Recognition, vol. 11, 91-93) have shown that 100% of M13 bacteriophages remain infective in 10% acetonitrile in 50 mM sodium phosphate, pH7 and in 30% acetonitrile in 50 mM sodium phosphate, pH7, while 50% remain infective in 99% acetonitrile in 50 mM sodium phosphate, pH7. For dimethyl formamide (DMF), Olofsson et al. 1998 have shown that 95% of M13 bacteriophages remain infective in 10% DMF, 50 mM sodium phosphate, pH7; 75% of bacteriophages remain infective in 30% DMF in 50 mM sodium phosphate, pH7; and 35% remain infective in 65% DMF in 50 mM sodium phosphate, pH7.

| Example | Medium composition | Outcome |
|---|---|---|
| T.1.2.2.1.2.1.1.2 | 10% acetonitrile; 50 mM sodium | >100 peptides that can bind to CNTs in a medium |
| | phosphate, pH 7 | composition containing ≥10% acetonitrile |
| T.1.2.3.1.2.1.1.2 | 30% acetonitrile; 50 mM sodium | >100 peptides that can bind to CNTs in a medium |
| | phosphate, pH 7 | composition containing ≥30% acetonitrile |
| T.1.2.4.1.2.1.1.2 | 99% acetonitrile; 50 mM sodium | >100 peptides that can bind to CNTs in a medium |
| | phosphate, pH 7 | composition containing ≥99% acetonitrile |
| T.1.2.5.1.2.1.1.2 | 10% dimethyl formamide; 50 mM | >100 peptides that can bind to CNTs in a medium |
| | sodium phosphate, pH 7 | composition containing ≥10% dimethyl formamide |
| T.1.2.6.1.2.1.1.2 | 30% dimethyl formamide; 50 mM | >100 peptides that can bind to CNTs in a medium |
| | sodium phosphate, pH 7 | composition containing ≥30% dimethyl formamide |
| T.1.2.7.1.2.1.1.2 | 65% dimethyl formamide; 50 mM | >100 peptides that can bind to CNTs in a medium |
| | sodium phosphate, pH 7 | composition containing ≥65% dimethyl formamide |
| T.1.2.8.1.2.1.1.2 | 10% propylene carbonate; 50 mM | >100 peptides that can bind to CNTs in a medium |
| | sodium phosphate, pH 7 | composition containing ≥10% propylene carbonate |
| T.1.2.9.1.2.1.1.2 | 30% propylene carbonate; 50 mM | >100 peptides that can bind to CNTs in a medium |
| | sodium phosphate, pH 7 | composition containing ≥30% propylene carbonate |
| T.1.2.10.1.2.1.1.2 | 99% propylene carbonate; 50 mM | >100 peptides that can bind to CNTs in a medium |
| | sodium phosphate, pH 7 | composition containing ≥99% propylene carbonate |
| T.1.2.11.1.2.1.1.2 | 10% chloroform; 50 mM sodium | >100 peptides that can bind to CNTs in a medium |
| | phosphate, pH 7 | composition containing ≥10% chloroform |
| T.1.2.12.1.2.1.1.2 | 30% chloroform; 50 mM sodium | >100 peptides that can bind to CNTs in a medium |
| | phosphate, pH 7 | composition containing ≥30% chloroform |
| T.1.2.13.1.2.1.1.2 | 99% chloroform; 50 mM sodium | >100 peptides that can bind to CNTs in a medium |
| | phosphate, pH 7 | composition containing ≥99% chloroform |
| T.1.2.14.1.2.1.1.2 | 10% caprolactone; 50 mM sodium | >100 peptides that can bind to CNTs in a medium |
| | phosphate, pH 7 | composition containing ≥10% caprolactone |
| T.1.2.15.1.2.1.1.2 | 30% caprolactone; 50 mM sodium | >100 peptides that can bind to CNTs in a medium |
| | phosphate, pH 7 | composition containing ≥30% caprolactone |
| T.1.2.16.1.2.1.1.2 | 99% caprolactone; 50 mM sodium | >100 peptides that can bind to CNTs in a medium |
| | phosphate, pH 7 | composition containing ≥99% caprolactone |

### Example M.3.5.0.8.0.0.0.0, Affinity Selection of a DNA-Encoded Small Molecule Library Encoding 85,000,000 Small Molecules

In this example, the library molecules are small organic molecules, and the tag (T) is DNA which encodes said small organic molecules. A library is generated using 4 rounds of building block addition as described in WO2007062664, example 2, using the following scheme which illustrates the synthesis of small molecules composed of 4 building blocks and encoded by 4 tags fragments:

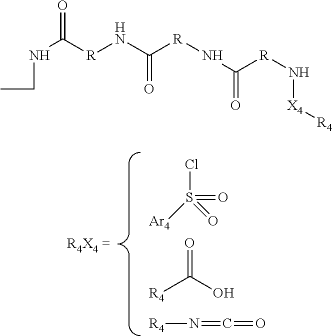

To identify ligands a fraction of the obtained bifunctional complexes is lyophilized and dissolved in 5 microliters thrombin buffer (137 mM NaCi, 2.7 mM KCl, 10 mM sodium phosphate, 0.1% PEG8000). 50 microliters streptavidin sepharose (Amersham Biosciences) slurry is washed in 4×100 microliters thrombin buffer and resuspended in 50 μl thrombin buffer. Then biotinylated thrombin (Novagen—2 units) is added to the streptavidin sepharose slurry and the slurry is incubated at 15 degrees C. with agitation (1400 rpm) for 30 minutes and subsequently washed 4 times with 100 microliters thrombin buffer. A 10 microliters Eppendorf tip is packed with glass wool up to the 2.5 μl mark. The streptavidin sepharose is applied to the tip and washed 3 times with 100 μl thrombin buffer by applying vacuum to the bottom end of the tip. The library is applied to the column and allowed to soak in. Then the column is washed 5 times with 100 μl thrombin buffer. Bifunctional complexes are eluted by applying 25 μl of a nanomolar ligand (100 μlM in PBS) to the column for 10 min followed by centrifugation of the column (1000 rcf for 30 seconds). An additional 25 μl PBS is applied to the column and spun through. The eluted material is re-applied to a fresh column. This cycle is repeated 4 times. A 10 μl sample of eluted material is used PCR using the forward and reverse primers 5′-CAAGTCACCAAGAATTCATG and 5′-TCTGGTGGTCTACGTGCTCT. The PCR product is clones and sequenced using standard methods.

### Example M.3.6.0.8.0.0.0.0, Affinity Selection of a DNA-Encoded Small Molecule Library Encoding 110,000,000,000 Small Molecules

The library is synthesized using the method described in example M.3.5.0.8.0.0.0.0. Briefly, a first set of building blocks (reactants) are loaded onto a display oligo (see WO2007062664 FIG. 1.2). The described general procedures are used in the following order: Position A building blocks (reactants) and A-tags: R1-P1-QC1-V1-T1-QC3-M1-V2-D1-V2-P3-V2-S1-V1. 576 position A building blocks (reactants) are used. The building block are trifunctional with one free —COOH reactive group, one Ns-protected amine and one Msec-protected amine. Position B building blocks (reactants) and B-tags: R1-P1-QC1-V1-T1-QC3-M1-V2-D1-V2-P3-V2-S1-V1. 576 position B building blocks (reactants) are used. Position C building blocks (reactants) and C-tags: (R1/R2/R3)-P1-QC1-V1-T1-QC3-M1-V2-D2-V2-P3-V2-S1-V1. 96 isocyanate building blocks (reactants) are used 192 sulfonoyl building blocks (reactants) are used 192 acylation building blocks (reactants) are used. Position D building blocks (reactants) and D-tags: (R1/R3/R4/R5)-P1-QC1-V1-T1-QC3-M1-V2-P3-V2-D1-V2. 96 aldehyde building blocks (reactants) are used 96 sulfonoyl building blocks (reactants) are used 192 halogenated heteroaromatic building blocks (reactants) are used 96 acylation building blocks (reactants) are used. The library is screened using unspecific elution (WO2007062664 example 7), identifiers are amplified and analyzed by ultra high-throughput sequencing. The sequencing revealed that identifiers containing the following tag combinations had been enriched:

### A543-B203-C131-D236

### A543-B203-C131-D236

### A543-B158-C131-D236

### A543-B158-C131-D122

### A543-B203-C131-D122

Ligands corresponding to said enriched identifiers are synthesized according to the method described in WO2007062664 example 5. The affinity of the ligands is determined to be between 1 nM and 10 μM

### Varying the Type of Material Used for Selection and Other Selection Parameters

Binding molecules are identified using the library described in Example M.3.6.0.8.0.0.0.0 and the screening method used in example T.1.1.1.1.1.1.1.1, except the material used to search for binding molecules is varied. TNT-buffer is used as the medium, sonication is used during washing as indicated, 0.02 mg/ml material is used, and the indicated method is used for elution;

| | | Sonication | | |
|---|---|---|---|---|
| | | during | | |
| Example | Material | washing? | Elution method | Outcome |
| M.3.6.1.1.4.1.1.3 | HiPco SuperPurified | No | Sonication | >100 small |
| | carbon nanotubes | | | molecules that |
| | (SWNTs, | | | can bind to |
| | (NanoIntegris | | | SWNTs |
| | Technologies, Inc., | | | |
| | Canada) | | | |
| M.3.6.1.2.4.1.2.4 | Metallic single wall | Yes | Glycine pH 2.2 | >100 small |
| | carbon nanotubes | | | molecules that |
| | (SWNTs, NanoIntegris | | | can bind to |
| | Technologies, Inc., | | | metallic SWNTs |
| | Canada) | | | |
| M.3.6.1.3.4.1.1.5 | Semiconducting single | No | Competitive | >100 small |
| | wall carbon nanotubes | | elution using | molecules that |
| | (SWNTs, NanoIntegris | | high | can bind to |
| | Technologies, Inc., | | concentration | semiconducting |
| | Canada) | | pyrene | SWNTs |
| M.3.6.1.4.4.1.2.3 | Plasma carbon | Yes | Extended | >100 small |
| | nanotubes (CNTs), | | sonication | molecules that |
| | NanoIntegris | | | can bind to |
| | Technologies, Inc., | | | plasma CNTs |
| | Canada | | | |
| M.3.6.1.5.4.1.1.6 | Multiwalled carbon | No | No elution; PCR | >100 small |
| | nanotubes (MWNT), | | of the DNA tag | molecules that |
| | Cheap Tubes, Inc., | | following by | can bind to |
| | USA | | sequencing | MWNTs |
| M.3.6.1.6.4.1.2.6 | Graphene Pure Sheets | Yes | No elution; PCR | >100 small |
| | MONO, NanoIntegris | | of the DNA tag | molecules that |
| | Technologies, Inc., | | following by | can bind to |
| | Canada | | sequencing | graphene |
| M.3.6.1.7.4.1.1.6 | Graphene nanoplatelets | No | No elution; PCR | >100 small |
| | (GNPs), Cheap Tubes, | | of the DNA tag | molecules that |
| | Inc., USA | | following by | can bind to |
| | | | sequencing | GNPs |

### Example M.3.7.0.9.0.0.0.0 Synthesis and Screening of a 7 Million-Member Scaffolded Library

Libraries are synthesized as described in Clark M A et. al 2009 (Acharya R A, Arico-Muendel C C, Belyanskaya S L, Benjamin D R, Carlson N R, et al. Design, synthesis and selection of DNA-encoded small-molecule libraries. Nat Chem Biol. 2009; 5: 647-654. doi:10.1038/nchembio.211). Specifically, two libraries are synthesized and screened; (1) a 7,000,000-member triazine library (DEL-A below) and (2) a 800,000,000-member, four-synthesis-cycles library (DEL-B, below).

### Synthesis Schemes of DEL-A and DEL-B:

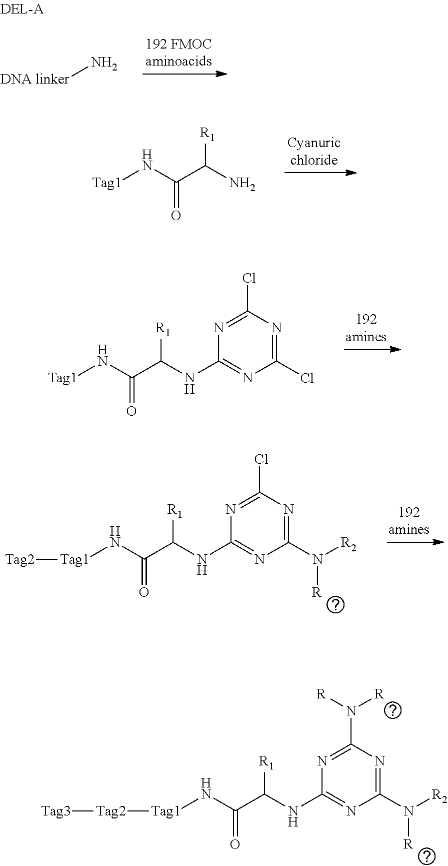

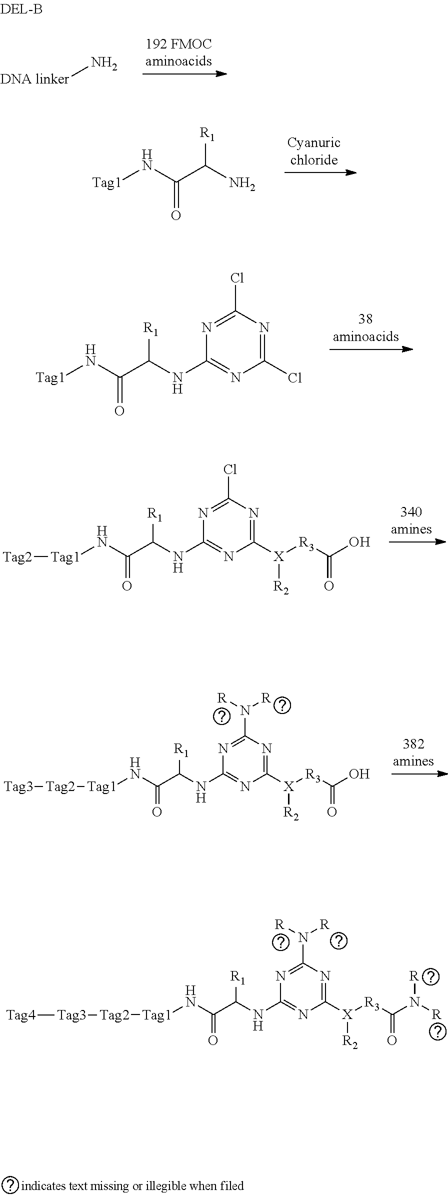

The libraries are screened as follows:

DEL A p38 screening. DEL library (5 nmol) is incubated with 500 nM His-tagged p38a protein (R&D Systems) in 60 μL selection buffer (50 mM Tris-HCl, pH 7.5, 150 mM NaCl, 0.1% tween-20, 1 mg/mL sheared salmon sperm DNA (Ambion) 1 mg/mL BSA (Ambion), and 1 mM βME) for 1 h at room temperature. The solution is incubated with 5 μL IMAC resin (Phynexus) for 5 min, then washed 10 times with 100 μL selection buffer. To elute the protein-bound library molecules, the resin is incubated with 60 μL imidazole buffer (50 mM Tris-HCl, pH 7.5, 150 mM NaCl, 0.1% tween-20, 1 mg/mL sheared salmon sperm DNA (Ambion) 1 mg/mL BSA (Ambion), 1 mM βME, and 100 mM imidazole) for 5 min. The eluant is heated for 5 min at 72° C. to denature the p38α protein. To allow for recapture of the denatured protein on IMAC resin, the elution is then diluted 10-fold in selection buffer to lower the imidazole concentration to 10 mM. The diluted elution is then incubated with IMAC resin (Phynexus) for 15 min to remove denatured protein and library molecules that bind to IMAC resin. Subsequent rounds of selection are performed by incubating the eluant from the previous round with 200 nM His-tagged p38α protein (Roche) in selection buffer for the second round of selection, and 200 nM His-tagged p38a protein (Roche) in selection buffer for the third round of selection, followed by the wash and elution steps described. Eluted molecules are used as templates for PCR amplification.

### Example M.3.7.0.9.0.0.0.0 Screening of a 800-Million-Member Scaffolded DNA-Encoded Library

Screening of the DEL-B described in example M.3.6.0.8.0.0.0.0; A 238 pmol aliquot of p38a (R&D Systems) is immobilized on 5 L IMAC resin (Phynexus). DEL B library (5 nmol) in 60 uL selection buffer (50 mM Tris-HCl, pH 7.5, 150 mM NaCl, 0.1% tween-20, 1 mg/mL sheared salmon sperm DNA (Ambion) 1 mg/mL BSA (Ambion), and 1 mM βME) is incubated with the immobilized p38a for 1 h at room temperature, then washed 10 times with 100 μL selection buffer. To elute the bound library molecules, the resin is incubated with 60 μL elution buffer at 72° C. for 10 min. The elution is then incubated twice with IMAC resin (Phynexus) for 15 min to remove denatured protein and library molecules that bind to IMAC resin. Subsequent rounds of selection are performed by incubating the elution from the previous round with fresh immobilized p38α (R&D Systems), followed by the wash and elution steps described. Decoding. After selection, library molecules are amplified by PCR (5 min at 95° C., then 20 cycles of 30 s at 92° C.; 15 s at 55° C.; 15 s at 72° C., followed by 10 min at 72° C.) using primers 5′O (5′-GCCTTGCCAGCCCGCTCAGTGACTCCCAAATCGATGTG-3′; 400 nM; IDT) and 3′O (5′GCCTCCCTCGCGCCATCAGGCAGGTGAAGCTTGTCTG-3′; 400 nM; IDT). The PCR products are purified to remove primers and nucleotides (PCR Clean-Up Kit, Qiagen) and sequenced (454 Life Sciences).

### Example M.3.9.0.10.0.0.0.0, Screening of a DNA-Encoded 4 Billion Member Small Molecule Library and Identification of Nanomolar Ligands

A 4 billion member library is synthesized as described in Deng et al. 2012, J. Med. Chem., 55, 7061-7079. Briefly, A 4,000,000,000-member library based on the triazine library concept reported earlier by the same group in Clark et al. 2009 is established and tested. The library is assembled from 192 FMOC-amino acids acylated on the DNA headpiece, followed by applying the triazine scaffold. This scaffold is assembled via the addition of cyanuric chloride to the FMOC-deprotected amines from Cycle 1. After that, 479 different amines are reacted. The next synthesis cycle incorporated 96 diamines, 60 of them containing a photolabile NVOC protecting group. The latter amines are further acylated with 173 carboxylic acids, subjected to reductive amination with 94 aldehydes, sulfonylated with 107 sulfonyl chlorides, and reacted with 85 isocyanates. Screening this library for ADAMTS-5 (aggrecanase-2, a member in the family of ADAMTS zinc metalloproteases cleaving the N-terminal interglobular domain of aggrecan) delivered multiple potent binders with affinities down to 30 nM paired with a clear structure-activity relationship of the hit series. The best hits showed strong selectivity for the ADAMTS-5 target compared to related metalloprotease enzymes like ADAMTS-1, ADAMTS-4, TACE, and MMP-13. Furthermore, they exhibited suppression of cytokine-mediated cartilage degradation in human osteoarthritis cartilage explants.

### Varying the Type of Material Used for Selection and Other Selection Parameters

Binding molecules are identified using the library described in Example M.3.6.0.8.0.0.0.0 and the screening method used in example T.1.1.1.1.1.1.1.1, except the material used to search for binding molecules is varied. Acetonitrile 99% is used as the medium, sonication is used during washing as indicated, 0.02 mg/ml material is used, and the indicated method is used for elution;

| | | Sonication | | |
|---|---|---|---|---|
| | | during | | |
| Example | Material | washing? | Elution method | Outcome |
| M.3.9.4.1.5.1.1.7 | HiPco SuperPurified | No | No elution; PCR | >100 small |
| | carbon nanotubes | | of the DNA tag | molecules that |
| | (SWNTs, | | following by | can bind to |
| | (NanoIntegris | | sequencing | SWNTs |
| | Technologies, Inc., | | | |
| | Canada) | | | |
| M.3.9.4.2.5.1.2.7 | Metallic single wall | Yes | No elution; PCR | >100 small |
| | carbon nanotubes | | of the DNA tag | molecules that |
| | (SWNTs, NanoIntegris | | following by | can bind to |
| | Technologies, Inc., | | sequencing | metallic SWNTs |
| | Canada) | | | |
| M.3.9.4.3.5.1.1.7 | Semiconducting single | No | No elution; PCR | >100 small |
| | wall carbon nanotubes | | of the DNA tag | molecules that |
| | (SWNTs, NanoIntegris | | following by | can bind to |
| | Technologies, Inc., | | sequencing | semiconducting |
| | Canada) | | | SWNTs |
| M.3.9.4.4.5.1.2.6 | Plasma carbon | Yes | Extended | >100 small |
| | nanotubes (CNTs), | | sonication | molecules that |
| | NanoIntegris | | | can bind to |
| | Technologies, Inc., | | | plasma CNTs |
| | Canada | | | |
| M.3.9.4.5.5.1.1.3 | Multiwalled carbon | No | Sonication | >100 small |
| | nanotubes (MWNT), | | | molecules that |
| | Cheap Tubes, Inc., | | | can bind to |
| | USA | | | MWNTs |
| M.3.9.4.6.5.1.2.4 | Graphene PureSheets | Yes | Glycine pH 2.2 | >100 small |
| | MONO, NanoIntegris | | | molecules that |
| | Technologies, Inc., | | | can bind to |
| | Canada | | | graphene |
| M.3.9.4.7.5.1.1.5 | Graphene nanoplatelets | No | Competitive | >100 small |
| | (GNPs), Cheap Tubes, | | elution using | molecules that |
| | Inc., USA | | high | can bind to |
| | | | concentration | GNPs |
| | | | pyrene | |

### Using Soluble Polymers as Components of the Medium.

Binding molecules are identified as described in example

M.3.9.4.1.5.1.1.7, except that the medium used is a mixture of CHCl₃ and Ethyl acetate with added soluble polymers as detailed in the following.

To provide soluble polymers, a first group of poly(3-hexylthiophene, P3HT)s is synthesized according to the catalyst transfer polymerization reaction method. These compounds, listed in Table 1 below, are tailor-made to their specific molecular weight. They have a very sharp molecular mass distribution and thus a very low polydispersity index (PDI). The number-average molecular weights (Mn) and the weight-average molecular weights (Mw) are measured by two methods; gel permeations chromatography (GPC) in tetrahydrofuran (THF) with polystyrene as calibration standard as well as matrix-assisted laser desorption ionization-time of flight mass spectroscopy (MALDI-TOF MS);

TABLE 1
Molecular Weights (MW) of Defined P3HTs Directly Synthesized
to Get Well-Controlled Molecular Weights

| Poly(3-hexylthiophene) | Mₙ [Da] | M_{w} [Da] | PDI | Mₙ | DP |
|---|---|---|---|---|---|
| Based on | GPC | GPC | GPC | MALDI-TOF MS | MALDI-TOF MS |
| 5 kDa | 5,100 | 6,300 | 1.22 | 3,200 | 19 |
| 11 kDa | 11,300 | 15,200 | 1.11 | 7,100 | 43 |
| 19 kDa | 18,600 | 21,600 | 1.16 | 12,400 | 74 |

Mₙ, number-average molecular weight; M_{w}, weight-average molecular Weight; PDI = M_{w}/Mₙ, polydispersity index; DP, degree of polymerization, i.e. the number of repeating units.

The second group of poly(3-hexylthiophene)s is synthesized according to the original McCullough route that does not allow for molecular weight control and that leads to broad molecular weight distributions. 18 In order to obtain different molecular weight samples, the initial molecular mass distribution is fractionated with different solvents as listed in Table 2:

TABLE 2
Poly(3-hexylthiophene) Fractionated from a Broad Molecular
Mass Distribution Sample with Different Solvents

| Poly(3-hexylthiophene) Fractionated With | Mₙ [Da] | M_{w} [Da] | PDI |
|---|---|---|---|
| Hexane | 5,600 | 6,600 | 1.18 |
| Dichloromethane | 13,800 | 20,400 | 1.48 |
| Chloroform | 19,00 | 25,600 | 1.35 |

Molecular weights measured with GPC (Mₙ, number-average molecular weight; M_{w}, weight-average molecular weight; PDI = M_{w}/Mₙ, polydispersity index).

An example of a mass spectrum poly(3-hexyl thiophene) is shown here:

(Scharsich et al., 2011, J. Polym Sci Part B: Polymer Physics 2012, 50, 442-453).

The low-molecular weight polymers are all soluble in 90% CHCl3, 10% Ethyl acetate without forming aggregates. Concentrations used are 0.25 mg/mol for the P3HT samples of defined MW, whereas 0.10 mg/mol is used for solutions of the extracted P3HT samples.

Soluble polymer preparations and other components of the medium used are:

| | | Mw [Da] | | | |
|---|---|---|---|---|---|
| | Soluble | (weight-average | Concentration | | |
| | polymer | Molecular | of soluble | | % Ethyl |
| Example | used | Weight) | polymer used | % CHCl₃ | acetate |
| M.3.9.4.1.5.1.1.7-1 | P3HT | 6300 | 0.25 mg/mol | 90 | 10 |
| M.3.9.4.1.5.1.1.7-2 | P3HT | 15200 | 0.25 mg/mol | 90 | 10 |
| M.3.9.4.1.5.1.1.7-3 | P3HT | 21600 | 0.25 mg/mol | 90 | 10 |
| M.3.9.4.1.5.1.1.7-4 | P3HT | 6600 | 0.10 mg/mol | 90 | 10 |
| M.3.9.4.1.5.1.1.7-5 | P3HT | 21600 | 0.25 mg/mol | 90 | 10 |
| M.3.9.4.1.5.1.1.7-6 | P3HT | 6600 | 0.10 mg/mol | 90 | 10 |

In all cases, the outcome is >100 small molecules which can bind SWNTs in the presence of P3HT.

Using Libraries with Library Molecules of Known Affinity

A library is synthesized as described in M.3.9.0.10.0.0.0.0, except that the molecules listed below are all included in the library. Molecules are linked to tags using standard chemical linking such that the affinity of the molecules towards the materials listed below are retained:

| | MW | K_{d}^{b} | K_{d}/MW | | |
|---|---|---|---|---|---|
| Ligand^{a} | [g/mol] | [M] | [M/Dal] | Material^{c} | Ref^{e} |
| QLMHDYR | 962.1 | 1.6E-05 | 1.7E-08 | PLLA (melt crystallization) | Matsuno et al. 2008 |
| QLMHDYR | 962.1 | 1.8E-04 | 1.8E-07 | PMMA (atactic) | Matsuno et al. 2008 |
| QLMHDYR | 962.1 | 1.5E-04 | 1.5E-07 | PLLA (amorphous) | Matsuno et al. 2008 |
| QLMHDYR | 962.1 | 6.7E-05 | 6.9E-08 | PLLA (layer by layer) | Matsuno et al. 2008 |
| QLMHDYR | 962.1 | 2.1E-05 | 2.2E-08 | PDLA | Matsuno et al. 2008 |
| CPTSTGQAC | 867 | 2.8E-07 | 3.2E-10 | Platinum | Seker et al. 2011 |
| CTLHVSSYC | 1,012 | 1.1E-06 | 1.1E-09 | Platinum | Seker et al. 2011 |
| RLNPPSQMD- | 1,399 | 8.3E-06 | 6.0E-09 | Silica | Seker et al. 2011 |
| PPF | | | | | |
| QTWPPPLWF- | 1,447 | 8.1E-07 | 5.6E-10 | Silica | Seker et al. 2011 |
| STS | | | | | |
| HTDWRLGT- | 1,533 | 1.3E-06 | 8.5E-10 | PPV (hyper-branched) | Eijima et al. 2010 |
| WHETS | | | | | |
| HTDWRLGT- | 1,533 | 1.9E-05 | 1.3E-08 | PPV (linear) | Eijima et al. 2010 |
| WHETS | | | | | |
| ELWSIDTSA- | 1,443 | 2.7E-05 | 1.9E-08 | PPV (hyper-branched) | Eijima et al. 2010 |
| HRK | | | | | |
| ELWSIDTSA- | 1,443 | 1.3E-05 | 9.0E-09 | PPV (linear) | Eijima et al. 2010 |
| HRK | | | | | |
| Sodium dodecyl- | 288.4 | 8.9E-04 | 3.1E-06 | SWNT (8,6) | Sim et al. 2013 |
| benzenesulfonate | | | | | |
| Sodium dodecyl- | 288.4 | 7.1E-04 | 2.5E-06 | SWNT (6,5) | Sim et al. 2013 |
| benzene sulfonate | | | | | |
| Sodium dodecyl- | 288.4 | 6.4E-04 | 2.2E-06 | SWNT (10,2) | Sim et al. 2013 |
| benzene sulfonate | | | | | |
| IFRLSWGTYFS | 1,377 | 2.0E-05 | 1.5E-08 | SWNT (HiPco, raw) | Li et al. 2013 |
| HWKHPWGA- | 1,534 | 5.0E-05 | 3.3E-08 | MWNT (array) | Wang et al. 2003 |
| WDTL | | | | | |
| ELWR | 602.7 | 6.3E-04 | 1.0E-06 | PMMA (isotactic) | Serizawa et al. 2007 |
| ELWRPTR | 957.1 | 3.6E-06 | 3.7E-09 | PMMA (isotactic) | Serizawa et al. 2007 |
| ELWRPTR | 957.1 | 1.5E-04 | 1.5E-07 | PMMA (syndiotactic) | Serizawa et al. 2007 |
| KPRSVSG | 965 | 5.4E-06 | 5.6E-09 | Ceramic fluorapatite | Islam et al. 2012 |
| KPRSVSG | 965 | 7.2E-06 | 7.5E-09 | Ceramic hydroxyl-apatite | Islam et al. 2012 |
| Pyrene | 202.3 | 6.1E-02 | 3.0E-04 | SWNT (plasma purified) | Juan et al. 2015 |
| Pyrene | 202.3 | 4.2E-02 | 2.1E-04 | SWNT (plasma purified) | Juan et al. 2015 |
| Pyrene | 202.3 | 4.8E-02 | 2.4E-04 | SWNT (plasma purified) | Juan et al. 2015 |
| Pyrene | 202.3 | 1.1E-01 | 5.5E-04 | SWNT (plasma purified) | Juan et al. 2015 |
| Pyrene | 202.3 | 2.2E-01 | 1.1E-03 | SWNT (plasma purified) | Juan et al. 2015 |
| Pyrene | 202.3 | 3.8E-04 | 1.9E-06 | SWNT (plasma purified) | Juan et al. 2015 |
| Pyrene | 202.3 | 2.4E-02 | 1.2E-04 | SWNT (6,5) | Juan et al. 2015 |
| Pyrene | 202.3 | 6.3E-01 | 3.1E-03 | SWNT (6,5) | Juan et al. 2015 |
| Pyrene | 202.3 | 6.3E-01 | 3.1E-03 | SWNT (6,5) | Juan et al. 2015 |
| Pyrene | 202.3 | 1.0E-03 | 4.9E-06 | SWNT (6,5) | Juan et al. 2015 |
| Pyrene diamine | 230 | 4.5E-03 | 2.0E-05 | SWNT (plasma purified) | Juan et al. 2015 |
| Pyrene diamine | 230 | 3.4E-02 | 1.5E-04 | SWNT (6,5) | Juan et al. 2015 |
| Pyrene phenyl | 240 | 1.1E-02 | 4.6E-05 | SWNT (plasma purified) | Juan et al. 2015 |
| ester | | | | | |
| Pyrene phenyl | 240 | 5.0E-02 | 2.1E-04 | SWNT (plasma purified) | Juan et al. 2015 |
| ester | | | | | |
| Dipyrene phenyl | 440 | 1.5E-04 | 3.5E-07 | SWNT (plasma purified) | Juan et al. 2015 |
| ester | | | | | |
| Dipyrene phenyl | 440 | 2.5E-04 | 5.7E-07 | SWNT (plasma purified) | Juan et al. 2015 |
| ester | | | | | |
| Bis-pyrene | 640 | 1.4E-04 | 2.2E-07 | SWNT (plasma purified) | Juan et al. 2015 |
| U-shape molecule | | | | | |
| MHGKTQATS- | 10,018 | 1.5E-10 | 1.5E-14 | Gold | Brown 1997 |
| GTIQS₇ | | | | | |
| DAEFRHDSGY- | 4,514 | 6.3E-18 | 1.4E-21 | Copper | Atwood et al. 2000 |
| EVHHQKLVFF- | | | | | |
| AEDVGSNKGA- | | | | | |
| IIGLMVGGVVIA | | | | | |
| DAEFRHDSGY- | 4,330 | 5.0E-11 | 1.2E-14 | Copper | Atwood et al. 2000 |
| EVHHQKLVFF- | | | | | |
| AEDVGSNKGA- | | | | | |
| IIGLMVGGVV | | | | | |
| CHKKPSKSC | 1,015 | 4.1E-09 | 4.0E-12 | Silica | Chen et al. 2006 |
| RKLPDAPGM- | 1,408 | 1.3E-05 | 9.4E-09 | Titanium | Sano et al. 2005 |
| HTW | | | | | |
| SCSDCLKSVD- | 1,946 | 2.5E-07 | 1.3E-10 | Titanium | Meyers et al. 2007 |
| FIPSSLASS | | | | | |
| YLTMPTP | 822 | 5.0E-12 | 6.1E-15 | Polystyrene (syndiotactic) | Serizawa et al. 2007 |
| FSWEAFA | 857 | 8.5E-11 | 9.9E-14 | Polystyrene (atactic) | Serizawa et al. 2007 |
| FSWEAFA | 857 | 3.4E-11 | 4.0E-14 | Polystyrene (isotactic) | Serizawa et al. 2007 |
| FSWEAFA | 857 | 6.1E-12 | 7.1E-15 | Polystyrene (syndiotactic) | Serizawa et al. 2007 |
| HLESTPG | 740 | 1.0E-10 | 1.4E-13 | Polystyrene (atactic) | Serizawa et al. 2007 |
| HLESTPG | 740 | 6.5E-11 | 8.8E-14 | Polystyrene (isotactic) | Serizawa et al. 2007 |
| HLESTPG | 740 | 7.9E-12 | 1.1E-14 | Polystyrene (syndiotactic) | Serizawa et al. 2007 |
| GETRAPL | 743 | 6.7E-11 | 9.0E-14 | Polystyrene (atactic) | Serizawa et al. 2007 |
| GETRAPL | 743 | 3.6E-11 | 4.9E-14 | Polystyrene (isotactic) | Serizawa et al. 2007 |
| GETRAPL | 743 | 6.7E-12 | 9.0E-15 | Polystyrene (syndiotactic) | Serizawa et al. 2007 |
| RHEPPLA | 819 | 5.6E-11 | 6.8E-14 | Polystyrene (atactic) | Serizawa et al. 2007 |
| RHEPPLA | 819 | 2.1E-11 | 2.6E-14 | Polystyrene (isotactic) | Serizawa et al. 2007 |
| RHEPPLA | 819 | 1.1E-11 | 1.3E-14 | Polystyrene (syndiotactic) | Serizawa et al. 2007 |
| GETQCAA | 679 | 3.4E-11 | 5.0E-14 | Polystyrene (atactic) | Serizawa et al. 2007 |
| YLTMPTP | 822 | 2.2E-11 | 2.6E-14 | Polystyrene (atactic) | Serizawa et al. 2007 |
| FPGRPSP | 757 | 3.1E-11 | 4.1E-14 | Polystyrene (atactic) | Serizawa et al. 2007 |
| HTAQSTA | 715 | 1.4E-11 | 2.0E-14 | Polystyrene (atactic) | Serizawa et al. 2007 |
| HKPDANR | 837 | 2.5E-11 | 3.0E-14 | PMMA (conditioned | Serizawa et al. 2007 |
| | | | | syndiotactic film) | |
| FPGHSGP | 698 | 1.0E-10 | 1.4E-13 | PMMA | Serizawa et al. 2007 |
| | | | | (non-conditioned | |
| | | | | syndiotactic film) | |
| THLPWQT | 882 | 6.7E-11 | 7.6E-14 | PMMA | Serizawa et al. 2007 |
| | | | | (non-conditioned | |
| | | | | syndiotactic film) | |
| GETQCAA | 679 | 7.4E-12 | 1.1E-14 | Polystyrene (syndiotactic) | Serizawa et al. 2007 |
| FPGRPSP | 757 | 8.5E-12 | 1.1E-14 | Polystyrene (syndiotactic) | Serizawa et al. 2007 |
| HTAQSTA | 715 | 1.0E-11 | 1.4E-14 | Polystyrene (syndiotactic) | Serizawa et al. 2007 |
| VKTQATSREE- | 9,433 | 1.0E-10 | 1.1E-14 | Zeolite | Nygaard et al. 2002 |
| PPRLPSKHRPG₄ | | | | | |
| PQAQDVELPQ- | 12,366 | 4.0E-11 | 3.2E-15 | SWNT | Brown et al. 2008 |
| ELQDQHREVEV₅ | | | | (HiPco, purified) | |

^{a}All ligands denoted by capital letters are peptide sequences; for polypeptides, the subscripts denote how many times the peptide sequence is repeated.
^{b}Kd is the dissociation constant.
^{c}MW-NT is multi walled carbon nanotube; SWNT is single walled carbon nanotube; PLLA is polylactic acid; PMMA is poly(methyl methacrylate); PDLA is poly-D-lactide; PPV is poly(p-phenylene vinylene); HDPE is high-density polyethylene; numbers in brackets after SWNTs denote chirality, e.g. (6,5).
^{d}Affinity parameter values with the unit L/kg are affinities expressed as the adsorption distribution coefficient; affinity parameter values with the unit M⁻¹ are affinities expressed as the affinity constant Kₐ, which is equal to 1/K_{d}; affinity parameter values with the unit kcal/mol are affinities expressed as the standard-state adsorption free energy values (ΔG° _{ads}); affinity parameter values with the unit L/mg are affinities expressed as Langmuir affinity constant (K_{L}).
^{e}References are Kragulj et al. 2013 (Kragulj, M., Trickovic, J., Dalmacija, B., Kukovecz A., Kónya, Z., Molnar, J., Roncevic, S. 2013. Molecular interactions between organic compounds and functionally modified multiwalled carbon nanotubes, Chemical Engineering Journal 225, 144-152); Ji et al. 2008 (Ji, L., Chen, W., Duan, L., Zhu, D. 2008. Mechanisms for strong adsorption of tetracycline to carbon nanotubes: A comparative study using activated carbon and graphite as adsorbents, Environmental Science & Technology, 43 (7), 2322-2327); Matsuno et al. 2008 (Matsuno, H., Sekine, J., Yajima, H., Serizawa, T. 2008. Biological Selection of Peptides for Poly(L-lactide) Substrates, Langmuir 24 (13), 6399-6403); Seker et al. 2011 (Seker, U. O. S., Demir, H. V. 2001. Material Binding Peptides for Nanotechnology, Molecules 16 (2), 1426-1451); Ejima et al. 2010 (Ejima, H., Matsuno, H., Serizawa, T. 2010. Biological Identification of Peptides that Specifically Bind to Poly(phenylene vinylene) Surfaces: Recognition of the Branched or Linear Structure of the Conjugated Polymer, Langmuir 26 (22), 17278-17285); Sim et al. 2013 (Sim, J., Oha, H., Kooa, E., Ju, S. 2013. Effect of tight flavin mononucleotide wrapping and its binding affinity on carbon nanotube covalent reactivities, Physical Chemistry Chemical Physics 15, 19169-19179); Li et al. 2013 (Li, Z., Uzawa, T., Tanaka, T., Hida, A., Ishibashi, K., Katakura, H., Kobatake, E., Ito, Y. 2013. In vitro selection of peptide aptamers with affinity to single-wall carbon nanotubes using a ribosome display, Biotechnology Letters 35, 39-45); Wang etal. 2003 (Wang, S., Humphreys, E. S., Chung, S., Delduco, D. F., Lustig, S. R., Wang, H., Parker, K. N., Rizzo, N. W., Subramoney, S., Chiang, Y., Jagota, A. 2003. Peptides with selective affinity for carbon nanotubes, Nature Materials 2, 196-200); Serizawa et al. 2007 (Serizawa, T., Sawada, T., Matsuno, H. 2007. Highly Specific Affinities of Short Peptides against Synthetic Polymers, Langmuir 23 (22), 11127-11133); Islam et al. 2012 (Islam, T., Bibi, N. S., Vennapusa, R. R., Fernandez-Lahore, M. 2012. Selection of ceramic fluorapatite-binding peptides from a phage display combinatorial peptide library: optimum affinity tags for fluorapatite chromatography, Journal of Molecular Recognition 26(8), 341-350); Juan et al. 2015 (de Juan, A., López-Moreno, A., Calbo, J., Orti, E., Pérez, E. M. 2015. Determination of association constants towards carbon nanotubes, Chemical Science 6,7008-7014); Aby et al. 2012 (Thyparambil, A. A., Wei, Y., Latour, R. A. 2012. Determination of Peptide-Surface Adsorption Free Energy for Material Surfaces Not Conducive to SPR or QCM using AFM, Langmuir 28 (13), 5687-5694); Chen et al. 2015 (Chen, X., Chen, B. 2015. Macroscopic and Spectroscopic Investigations of the Adsorption of Nitroaromatic Compounds on Graphene Oxide, Reduced Graphene Oxide, and Graphene Nanosheets, Environmental Science and Technology 49 (10), 6181-6189); Brown 1997 (Brown, S. 1997. Metal-recognition by repeating polypeptides, Nature Biotechnology 15,269-272); Atwood et al. 2000 (Atwood, C. S., Scarpa, R. C., Huang, X., Moir, R. D., Jones, W. D., Fairlie, D. P., Tanzi, R. E., Bush, A. I. 2000. Characterization of Copper Interactions with Alzheimer Amyloid β Peptides, Journal of Neurochemistry 75 (3), 1219-1233); Chen et al. 2006 (Chen, H., Su, X., Neoh, K., Choe, W. 2006. QCM-D Analysis of Binding Mechanism of Phage Particles Displaying a Constrained Heptapeptide with Specific Affinity to SiO₂ and TiO₂, Analytical Chemistry 78(14), 4872-4879); Sano et al. 2005 (Sano, K., Ajima, K., Iwahori, K., Yudasaka, M., Iijima, S., Yamashita, I., Shiba, K. 2005. Endowing a Ferritin-Like Cage Protein with High Affinity and Selectivity for Certain Inorganic Materials, Small 1 (8-9), 826-832); Meyers et al. 2007 (Meyers, S. R., Hamilton, P. T., Walsh, E. B., Kenan, D. J., Grinstaff, M. W. 2007. Endothelialization of Titanium Surfaces, Advanced Materials 19,2492-2498); Serizawa et al. 2007 (Serizawa, T., Techawanitchai, P., Matsuno, H. 2007. Isolation of Peptides that Can Recognize Syndiotactic Polystyrene, ChemBioChem 8 (9), 989-993); Nygaard et al. 2002 (Nygaard, S., Wendelbo, R., Brown, S. 2002. Surface-Specific Zeolite-Binding Proteins, Advanced Materials 14 (24), 1853-1856); Brown et al. 2008 (Brown, S., Jespersen, T. S., Nygaard, J. 2008. A Genetic Analysis of Carbon-Nanotube-Binding Proteins, Small 4 (4), 416-420).

The library is screened on the following targets in parallel (as described in example M.3.10.0.11.0.0.0.0); using appropriate buffers PLLA (melt crystallization), PMMA (atactic), PLLA (amorphous), PLLA (layer by layer), PDLA, Platinum, Silica, PPV (hyper-branched), PPV (linear), SWNT (8,6), SWNT (6,5), SWNT (10,2), SWNT (HiPco, raw), MWNT (array), PMMA (isotactic), PMMA (syndiotactic), Ceramic fluorapatite, Ceramic hydroxyl-apatite, SWNT (plasma purified), Gold, Copper, Titanium, Polystyrene (syndiotactic), Polystyrene (atactic), Polystyrene (isotactic), Polystyrene (atactic), PMMA (conditioned syndiotactic film), PMMA (non-conditioned syndiotactic film), Zeolite, SWNT (HiPco, purified), boron nitride nanotubes (BNNTs), and boron nitride sheets (BNS), and graphene, and molecules with affinity said materials are identified.

### Example M.3.10.0.11.0.0.0.0, Screening of a DNA-Encoded Macrocyclic Library and Identification of Ligands with Nanomolar Affinity

A library with the general structure shown below is synthesized as described in Kleiner et al. 2010, JACS, 132, 11779-11791, and nanomolar ligands are identified. Briefly, the DNA-templated small-molecule macrocycle library used in this in Vitro selection effort is generated in three DNA-templated reactions followed by a nontemplated Wittig macrocyclization reaction to generate the final product. Each DNA-templated library synthesis step relied on a set of 12 DNA-linked building blocks comprising both natural and non-natural amino acids. These 36 building blocks together with eight variable diamino-acid scaffolds result in a theoretical diversity of 13 824 DNA-templated macrocycles. The fidelity of DNA-templated macrocycle library synthesis is extensively characterized by high-resolution LC-MS analysis of sublibraries and by gel electrophoresis and LC-MS analysis of reactions containing all possible building blocks and scaffolds. A stock solution of GST-labeled protein target is diluted in protein-binding buffer (50 mM Tris-HCl, pH 7.5, 50 mM NaCl, 10 mM beta-mercaptoethanol) to a final concentration of 1 μM. The protein target (200 pmol) is incubated with 2 μL of MagneGST. (Promega) glutathione-linked magnetic particles for 1 h at 4° C. with gentle shaking. The beads are washed three times with 100 μL of TBST buffer supplemented with 1 M NaCl, followed by two washes with 300 μL of protein-binding buffer. The beads are then diluted in 20 μL selection buffer (TBST, 3 mg/mL yeast tRNA, 1 mM DTT) and 10 μL of resulting protein bead solution is combined with 1 pmol of a nonbinding DNA sequence and 1/10, 1/100, 1/1000, or 1/10 000 pmol of the known protein-binding ligand conjugated to DNA. After removing unbound molecules, the beads are washed three times with 200 μL of selection buffer, and bound molecules are eluted in 20 μL of 0.1 mM glutathione in 50 mM Tris pH 8.0 buffer for 15 min. Of this eluant, 5 μL is subjected to PCR amplification. In Vitro selections of the 13 824-membered library are conducted exactly as described above for the positive control selections. The library is screened on the following targets in parallel; Akt3 AMPK, ERBB4, MK2, p38R, MKK6, Piml, Src, VEGFR2, DEP1, MEG2, PRL2, Cdc42, H-Ras-V12, Erbin, Dvl1-3, Bcl-xL, BIR3, (XIAP), Abl1, Abl2, P85RN, PPARδ, P85RC, P55γC, RhoA, γ1-syntrophin, Magi1, MUPP1, PAR6B, PSD95, RGS3, SAP97, Semcap3, Shank3. For each selection, 5 pmol of library is used. PCR amplification of selection survivors is performed with 5′-barcoded forward and reverse primers. Barcoded PCR amplicons are quantitated using Picogreen dsDNA quantitation reagent (Invitrogen), mixed in equimolar amounts, and submitted for high-throughput DNA sequencing. The most potent molecules, macrocycles with building blocks/fragments trans-A10-B1-C5-D6 and cis-A11-B1-C5-D7, which share the furylalanine (B1) and cyclopropylalanine (C5) building blocks, but otherwise display significant structural diversity, inhibited Src with IC50 values of 680 and 960 nM, respectively. The following scheme shows the encoding layout of the library.

### Example M.3.11.0.12.0.0.0.0, Screening of a DNA-Encoded Small Molecule Library Synthesized Using Chemical Ligation—and Identification of 2 nM Affinity Ligands

A library is synthesized and screened as described in Litovchick (2015) DOi: 10.1038/srep10916.

Scheme for encoded library synthesis. a) 2,259 5′-azido-3′-TIPS propargyl tags, Cu(OAc)2, sodium ascorbate, TBTA; b) 2,259 primary amines, NaBH3CN, 60 deg. C.; c) TBAF; d) 666 5′-azido-3′-TIPS propargyl tags, Cu(OAc)2, sodium ascorbate, TBTA; e) 3×222 bromoaryl acids, DMT-MM f) 669 5′-azido-3′-TIPS propargyl tags, Cu(OAc)2, sodium ascorbate, TBTA; g) 667 boronic acids and esters, Pd(PPh3)4, CsCO3. Remaining 2 wells are encoded nulls, and received no reagent or palladium catalyst and no boronate, respectively. Library synthesis is based on a set of bromoarylcarboxylic acids. The synthetic route is shown in the scheme above. In the first cycle of library synthesis, the starting material is split into 2,259 wells for tagging by CuAAC. After chemical ligation, 2,259 primary amines are installed onto the aldehyde by reductive amination. After pooling the wells, the TIPS group is removed by treatment with TBAF. In the next diversity-generating step, the material is split into 666 wells, tagged by CuAAC, and then acylated with 3×222 bromoarylcarboxylates. After pooling and deprotection, a final diversification step is conducted with 669 tags and 667 boronic acid/boronate ester building blocks, which are installed by Suzuki cross-coupling (the extra tags are used to encode the products of reaction failure). Installation of boronates onto oligonucleotides by Suzuki coupling has been shown previously, including in the context of encoded libraries. We adapted procedures commonly used for Suzuki coupling under aqueous conditions. After completion of the final step, the wells are pooled and the library purified by reverse-phase HPLC. The recovered yield of library after HPLC purification following the Suzuki chemistry is 3-fold lower than that in the previous two steps (17%, compared to 53% and 61% respectively). We believe the loss is related to the lower yield of the Suzuki reaction and the generally lower recovery of library DNA we've observed after transition-metal mediated chemistries. Whatever the source of the losses, they do not appear to arise from the chemical ligation protocol, as they are not observed in the first two synthesis cycles. Prior to selection, the 334 million compound library is subjected to TIPS deprotection and ligated by CuAAC with a terminal self-priming hairpin oligonucleotide. Primer extension with Klenow yielded library with a double-stranded encoding oligonucleotide, suitable for affinity selection. We subjected the library to affinity-mediated selection against the target soluble epoxide hydrolase (sEH). This enzyme is involved in the conversion of epoxyeicosatrienoic acids (EETs) to dihydroxyeicosatrienoic acids (DHETs). Various studies have shown that EETs have anti-inflammatory properties, and so it has been suggested that inhibitors of sEH could have beneficial effects in COPD, cardiovascular disease, and even diabetes. Researchers at GSK have reported on sEH inhibitors that show beneficial effects in a cigarette smoke exposure mouse model28,29. These compounds are derived from leads originally discovered using a DNA-encoded library platform.

Selections are performed by conventional methods as described elsewhere. Library is incubated with His-tagged sEH protein in solution, followed by protein capture using an immobilized-metal affinity chromatography matrix (IMAC) resin. After washing, bound library members are eluted by heat denaturation. The resulting population of enriched library members is subjected to a second round of selection. The selection output is amplified by PCR and sequenced using an Illumina HiSeq 2500 instrument; the sequencing output is translated into chemical identifiers.

| Cycle A | Cycle B | Cycle C |
|---|---|---|
| 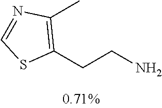 | 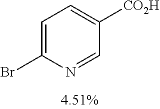 | 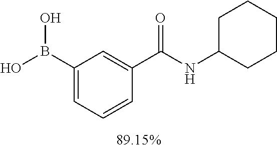 [SMILES: O=C(NC1CCCCC1)c1cccc(B(O)O)c1] |
| 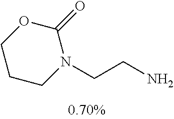 | 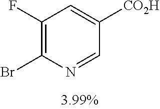 [SMILES: O=C(O)c1cnc(Br)c(F)c1] | 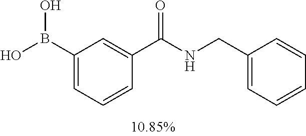 [SMILES: O=C(NCc1ccccc1)c1cccc(B(O)O)c1] |
| 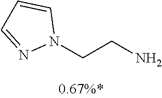 | 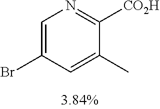 | |

Prevalance of the most abundant building blocks in each position for the sEH-selected family. The combination of the starred building blocks is the single most abundant trisynthon in the family, corresponding to compound 12.

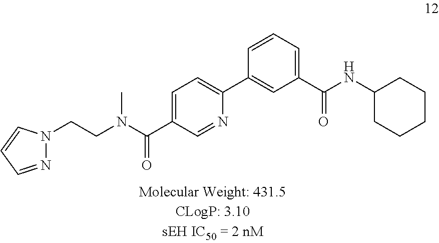 [SMILES: CN(CCn1cccn1)C(=O)c1ccc(-c2cccc(C(=O)NC3CCCCC3)c2)nc1]

The most abundant trisynthon in the family is the combination of N-aminoethyl pyrazole at Cycle A, 5-bromonicotinic acid at Cycle B, and N-cyclohexyl-3-boronylbenzamide at Cycle C. The corresponding compound 12 (with a methyl group in place of the DNA linker) is synthesized and assayed for sEH inhibition, and found to have an IC50 value of 2 nM. The building block combination that comprises compound 12 is observed thirteen times among the 147,885 sequence reads generated for the selected library, and not at all in the 2,037,412 sequence reads generated for the naïve library. This represents an approximately 30,000-fold enrichment over two affinity-mediated selection steps when compared to the average within the naïve library.

Uses and Applications of Molecules of the Present Invention

Molecules identified according to the present invention may be used to dissociate carbon nanotubes from aggregates as described in Brown S. et al. 2010 Advanced Materials Volume 23, Issue 1, pages 132-135, Jan. 4, 2011. Molecules identified according to the present invention may be used for sorting CNTs, graphene or any other fullerene. Molecules identified according to the present invention may be used for coating CNTs, graphene, or any other fullerene. Molecules identified according to the present invention may be used for assembling a layer (e.g. Teflon) on another layer (e.g. steel). Molecules identified according to the present invention may be used for noncovalent or covalent cross-linking of materials. Molecules identified according to the present invention may be used for catalysing growth of CNTs or graphene. Molecules identified according to the present invention may be used for fabricating cathodes

Methods described herein may be used to identify molecules which can catalyze or accelerate a process, for example catalysis of a chemical reaction, growth of a carbon nanotube. Examples of molecules which may be present in molecule libraries of the present invention are: SiO(2) nanoparticles, Al(2)O(3), TiO(2), patterned Co—Mo salts, particles of Ni, Co, or Fe, In, Cu, Ag and Au, Pt/Rh/Re such as (18.98:3.22:0.8 wt %) and Ni/Co such as (10:10 wt %), Si/SiO2/Al2O3, zirconium oxide. Pt, Pd, Mn, Mo, Cr, Sn, Au, Mg, Al, acetylene, methyl acetylene, and vinyl acetylene.

### EMBODIMENTS

### Embodiment 1

A method for identification of molecules with desired characteristics, comprising the steps of

A. Providing a library of molecules
B. Providing a medium
C. Providing a surface or material
D. Combining one or more of said library, medium, surface and/or material
E. Optionally performing one or more manipulations of said library, medium, surface and/or material
F. Optionally, partitioning or isolating a fraction of said library
G. Optionally amplifying or copying the whole or a part of one or more molecules
H. Optionally identifying one or more characteristics of one or more library molecules

### Wherein

steps A, B, C, D, E, F, G, and H are performed simultaneously or sequentially in any possible order;
and wherein
steps A, B, C, D, E, F, G, and H are performed one or more times

### Embodiment 2

The method of Embodiment 1, wherein said medium comprises a polymer or a

### Embodiment 3

The method of Embodiment 2, wherein said repeat unit structurally or functionally resembles a surface or material

### Embodiment 4

The method of Embodiment 2, wherein said repeat unit structurally or functionally resembles a surface or material

### Embodiment 5

The method of Embodiment 2, wherein said repeat unit is derived from a surface or material

### Embodiment 6

The method of Embodiment 1, wherein said medium structurally or functionally resembles a surface or a material

### Embodiment 7

The method of Embodiment 1, wherein said medium and said material is chosen such that the number of hydrogen bond acceptors per cubic nanometer of the medium divided by the number of hydrogen bond acceptors per cubic nanometer of the material; and/or the number of hydrogen bond donors per cubic nanometer of the medium divided by the number of hydrogen bond donors per cubic nanometer of the material; and/or the dielectric constant of the medium divided by the dielectric constant of the material is 1E−3 to 1E−2, 1E−2 to 1E−1, 1E−1 to 1E+1, 1E+1 to 1E+2, 1E+2 to 1E+3.

### Embodiment 8

The method of Embodiment 1, wherein said medium and said material is chosen such that such that the pH of the medium divided by the pH of the material is 1E−3 to 1E−2, 1E−2 to 1E−1, 1E−1 to 1E+1, 1E+1 to 1E+2, 1E+2 to 1E+3.

### Embodiment 9

The method of Embodiments 1, wherein said surface or material is a polymer Embodiment 10. The method of Embodiment 1, wherein said surface or material is a polymer chosen from; Polyethylenes: Polyethylene (PE), Low density polyethylene (LDPE), High density polyethylene (HDPE), Linear low density polyethylene (LLDPE), Crosslinked polyethylene (XLPE), Ultra High Molecular Weight Polyethylene (UHMWPE). Other Polyolefins: Polypropylene (PP), Biaxially-oriented polypropylene, Polybutylene (PB), Polyisobutene (PIB) Polyacrylates: Polymethyl methacrylate (PMMA), polymethyl acrylate (PMA), hydroxyethyl methacrylate (HEMA), Sodium polyacrylate. Polystyrenes: Polystyrene (PS), High impact polystyrene (HIPS), Extruded polystyrene (XPS), Expanded Polystyrene, Polyesters: Polyethylene terephthalate (PET). Polysulfones: Polysulfone (PSU), Polyarylsulfone (PAS), Polyethersulfone PES, Polyphenylsulfone (PPS). Polyamides: Polyamide (PA), polyphthalamide (PPA), Bismaleimide (BMI), urea formaldehyde (UF) Polyurethanes: Polyurethane (PU), Polyisocyanurate (PIR). Chloropolymers: Polyvinyl chloride (PVC), Polyvinylidene dichloride (PVDC). (Chloro)fluoropolymers: Fluoropolymer (FE), Polytetrafluoroethylene (PTFE), Polyvinylidene difluoride (PVDF), Polychlorotrifluoroethlyene (PCTFE), Ethylene chlorotrifluoroethlyene (ECTFE). Other Homopolymers: Polycarbonate (PC), Polylactic acid (PLA), Polyacrylamide (PAM), Polyetheretherketone (PEEK). Other Copolymers: Acrylonitrile butadiene styrene (ABS), Polybutadiene acrylonitrile (PBAN). Polymers suitable for use according to the present invention may also be selected from; BMI Bismaleimide, Cellulose acetate, Cellulose acetate butyrate, Cellulose acetate propionate, Cellulose propionate, Cellulosics, cyamelide, ECTFE Ethylene chlorotrifluoroethlyene, ETFE Ethylene TetrafluoroEthylene, Ethyl cellulose, FEP Fluorinated Ethylene Propylene, HDPE High density polyethylene, HEMA hydroxyethyl methacrylate, HEMA hydroxyethyl methacrylate, LDPE Low density polyethylene, LLDPE Linear low density polyethylene, Methyl methacrylate, PA Polyamide, PAM polyacrylamide, PAM polyacrylamide, PAN polyacrylonitrile, PAN polyacrylonitrile, PAS Polyarylsulfone, PB Polybutylene, PBAN polybutadiene acrylonitrile, PBAN polybutadiene acrylonitrile, PBT Polybutylene terephthalate, PCT Type, PCTA Type, PCTFE Polychlorotrifluoroethylene (Teflon), PCTG Type, PE Polyethylene, PEBA Polyether Block Amide elastomer, PES Polyethersulfone, PET Polyethylene terephthalate, PETG Type, PEX Crosslinked high density polyethylene, PFA Perfluoroalkoxyethylene, PIB Polyisobutene, PMA polymethyl acrylate, PMA polymethyl acrylate, PMMA polymethyl methacrylate, PMMA polymethyl methacrylate, Polyacrylate, Polyaryletherketones, polyurea, Polyvinyl, Polyvinyl acetate, Polyvinyl alcohol, Polyvinyl carbazole, Polyvinyl chloride, PP Polypropylene, PPA polyphthalamide, PPS Polyphenylsulfone, PSU Polysulfone, PTFE Polytetrafluoroethylene, PVDF Polyvinylidene fluoride, PVF Polyvinyl fluoride, Sodium polyacrylate, UF urea formaldehyde, UHMWPE Ultra high molecular weight polyethylene, XLPE Crosslinked Polyethylene, Alkyd, Allyl Diglycol Carbonate Monomer, Bismaleimide (BMI), DAIP (Diallyl Isophthalate), DAP (Diallyl Phthalate), DCPD Resin, Epoxy, Furan, Melamine, Melamine/Phenolic, Phenolic, Polybutandiene, Polyester (Unsaturated), Bulk Molding Compounds (BMC), Gel Coats, Granular Molding Compounds (GMC), Resins, Sheet Molding Compounds (SMC), Thick Molding Compounds (TMC), Polyimide (Thermosetting), Silicone, Urea, Urethane Chemicals, Vinyl Ester, Molding Compounds, ABS, acrylonitrile-butadiene-styrene copolymer; ACMA, acrylonitrile-methyl acrylate copolymer; AIBN, 2,2-azobisisobutyronitrile; ATRP, atom transfer radical polymerization; CNT, carbon nanotube; CPP, chlorinated polypropylene; DWCNT, double-walled carbon nanotube; EMMA, ethyl-methyl methacrylate copolymer; EPDM, ethylene-propylene-diene rubber; EVA, ethylene-vinyl acetate copolymer; EVOH, ethylene-vinyl alcohol copolymer; HDPE, high-density polyethylene; HMW, high molecular weight; LDPE, low density polyethylene; LMW, low molecular weight; MA, maleic anhydride; MBMA, methyl-butyl methacrylate copolymer; MDPE, medium density polyethylene; MEMA, methyl-ethyl methacrylate copolymer; MPTS, methacryloxypropyltrimethoxysilane; MWCNT, multi-walled carbon nanotube; NMP, nitroxide-mediated polymerization; NMR, nuclear magnetic resonance; P3HT, poly(3-hexylthiophene); P3OT, poly(3-octylthiophene); PA, poly-acetylene; PAA, poly(acrylic acid); PABS, poly(m-aminobenzene sulfonic acid); PAM, polyacrylamide; PAMAM, poly(amidoamine); PAN, polyacrylonitrile; PANI, polyaniline; Parmax, poly(benzoyl-1,4-phenylene)-co-(1,3-phenylene); PBA, polybutyl acrylate; PtBA, poly(tert-butyl acrylate); PBMA, poly(butyl methacrylate); PBO, poly(phenylenebenzobisoxazole); PBT, poly(butyl terephthalate); PC, polycarbonate; PCL, polycaprolactone; PDEAEMA, poly[2-(diethylamino)ethyl methacrylate]; PDI, polydispersity index; PDMEMA, poly[2-(dimethylamino)ethyl methacrylate]; PDMS, polydimethylsiloxane; PDPA, polydiphenylamime; PE, polyethylene; PEG, polyethyleneglycol; PEI, polyethyleneimine; PEMA, poly(ethyl methacrylate); PEO, polyethyleneoxide; PET, poly(ethyl terephthalate); PETI, phenylethynyl-terminated imide; PGMA, poly(glycerol monomethacrylate); PHEMA, poly(2-hydroxyethyl methacrylate); PHET, poly[3-(2-hydroxyethyl)-2,5-thienylene]; PHPMA, poly[N-(2-hydroxypropyl)methacrylamide]; PI, polyimide; PIMA, poly(imidazolium methacrylate); PLLA, poly(1-lactic acid); PLLA-g-AA, poly(1-lactic acid) grafted with poly(acrylic acid) chains; PMDMAS, poly[3-(N-(3-methacrylamidopropyl)-N,N-dimethyl)ammoniopropanatesulfonate]; PMMA, poly(methyl methacrylate); PMMAHEMA, poly[(methyl methacrylate)-co-(2-hydroxyethyl methacrylate)]; PmPV, poly(m-phenylenevinylene-co-2,5-dioctoxy-p-phenylenevinylene); PNIPAAm, poly(N-isopropylacrylamide); PP, polypropylene; PPE, poly(p-phenylene ethynylene); PPEI-EI, poly(propionylethylenimine-co-ethylimine); PPS, poly(phenylene sulfide); PPY, polypyrrole; PS, polystyrene; PSS, poly(sodium 4-styrenesulfonate); PSV, poly(styrene-co-p-(4-(4-vinylphenyl)-3-oxobutanol)); PTH, polythiophene; PU, polyurethane; PVA, poly(vinyl alcohol); PVAc, poly(vinyl acetate); PVAc-VA, poly(vinyl acetate-co-vinyl alcohol); PVC, poly(vinyl chloride); PVDF, poly(vinylidene fluoride); PVK, poly(N-vinyl carbazole); PVKV, poly(N-vinyl carbazole-co-p-(4-(4-vinylphenyl)-3-oxobutanol)); PVP, polyvinylpyrrolidone; P2VP, poly(2-vinylpyridine); P4VP, poly(4-vinylpyridine); RAFT, reversible addition-fragmentation chain transfer polymerization; ROP, ring opening polymerization; SAN, styrene-acrylonitrile copolymer; SBA, styrene-butyl acrylate copolymer; SBBS, styrene-butadiene-butylene-styrene copolymer; SBR, styrene-butadiene rubber; SCMS, styrene-p-chloromethylstyrene copolymer; SE, silicone elastomer; SEC, size exclusion chromatography; SIBS, poly(styrene-b-isobutylene-b-styrene); SMA, styrene maleic anhydride copolymer; STM, scanning tunneling microscopy; SWCNT, single-walled carbon nanotube; TDI, toluene diisocyanate; TEMPO, 2,2,6,6-tetramethylpiperidinyl-1-oxy; TGA, thermogravimetric analysis; THF, tetrahydrofuran; UHMWPE, ultra high molecular weight polyethylene; WBPU, waterborne polyurethane.

### Embodiment 11

The method of Embodiment 1, wherein said surface or material is hydrophobic

### Embodiment 12

The method of Embodiment 1, wherein said medium comprises one or more organic solvents

### Embodiment 13

The method of Embodiment 1, wherein said medium comprises one or more pH buffering entities

### Embodiment 14

The method of Embodiment 1, wherein said library comprises organic molecules

### Embodiment 15

The method of Embodiment 1, wherein said library comprises Amino acids, such as L-amino acids, D-amino acids, alpha amino acids, beta amino acids, gamma amino acids, essential amino acids, non-essential amino acids, imino acids, N-substituted L-amino acids, N-substituted D-amino acids, N-substituted alpha amino acids, N-substituted beta amino acids, N-substituted gamma amino acids, N-substituted essential amino acids, N-substituted non-essential amino acids, N-substituted imino acids

### Embodiment 16

The method of Embodiment 1, wherein said library comprises peptides, such as peptides comprising L-amino acids, D-amino acids, alpha amino acids, beta amino acids, gamma amino acids, essential amino acids, non-essential amino acids, imino acids, N-substituted L-amino acids, N-substituted D-amino acids, N-substituted alpha amino acids, N-substituted beta amino acids, N-substituted gamma amino acids, N-substituted essential amino acids, N-substituted non-essential amino acids, and/or N-substituted imino acids

### Embodiment 17

The method of Embodiment 1, wherein said library comprises proteins, such as Antibodies, Antibody fragments, VhH domains, V-NAR domains, VH domains, VL domains, Camel Igs, IgNAR, IgG, Fab, Fab2, Fab3, Bis-scFv, Minibody (bivalent), scFV, Triabody

### Embodiment 18

The method of Embodiment 1, wherein said library comprises Carbohydrates, such as linear carbohydrates, branched carbohydrates, monosaccharides, disaccharides, oligosaccharides, polysaccharides

### Embodiment 19

The method of Embodiment 1, wherein said library comprises Lipids, such as Sterols, fatty acids, waxes, monoglycerides, diglycerides, phospholipids, fatty acyls, glycerolipids, glycerophospholipids, sphingolipids, saccharolipids, polyketides

### Embodiment 20

The method of Embodiment 1, wherein said library comprises Nucleic acids, such as RNA, mRNA, rRNA, tRNA, tmRNA, snRNA, snoRNA, scaRNA, gRNA, aRNA, crRNA, ncRNA

### Embodiment 21

The method of Embodiment 1, wherein said library comprises drug-like fragments

### Embodiment 22

The method of Embodiment 1, wherein said library comprises non-drug-like fragments

### Embodiment 23

The method of Embodiment 1, wherein said library comprises mixed organic/inorganic molecules

### Embodiment 24

The method of Embodiment 1, wherein said library comprises Inorganic molecules

### Embodiment 25

The method of Embodiment 1, wherein said molecules have a molecular weight (in daltons) from 2E+0 to 1E+1, 1E+1 to 3E+1, 3E+1 to 9E+1, 9E+1 to 3E+2, 3E+2 to 8E+2, 8E+2 to 2E+3, 2E+3 to 7E+3, 7E+3 to 2E+4, 2E+4 to 7E+4, 7E+4 to 2E+5, 2E+5 to 6E+5, 6E+5 to 2E+6, 2E+6 to 5E+6, 5E+6 to 2E+7, 2E+7 to 5E+7, 5E+7 to 1E+8

### Embodiment 26

The method of Embodiment 1, wherein said molecules have a number of atoms from 2 to 4, 4 to 7, 7 to 11, 11 to 17, 17 to 27, 27 to 42, 42 to 68, 68 to 108, 108 to 172, 172 to 275, 275 to 440, 440 to 704, 704 to 1126, 1126 to 1802, 1802 to 2883, 2883 to 4612

### Embodiment 27

The method of Embodiment 1, wherein said molecules have the following number of hydrogen bond acceptors such as 0, from 1 to 2, 2 to 3, 4 to 5, 5 to 6, 6 to 7, 7 to 8, 8 to 9, 9 to 10, 10 to 12, 12 to 14, 14 to 16, 16 to 18, 18 to 20, 20 to 22, 22 to 24, 24 to 26, 26 to 28, 28 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95 hydrogen bond acceptors.

### Embodiment 28

The method of Embodiment 1, wherein said molecules have a number of rotatable bonds from 0, from 1 to 2, 2 to 3, 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, 20 to 21, 21 to 22, 22 to 23, 23 to 24, 24 to 25, 25 to 26, 26 to 27, 27 to 28, 28 to 29, 29 to 30 rotatable bonds.

### Embodiment 29

The method of Embodiment 1, wherein said molecules are not tagged.

### Embodiment 30

The method of Embodiment 1, wherein said molecules are tagged.

### Embodiment 31

The method of Embodiment 30, wherein said tags comprise organic tags, poly-amino acids, 13C-labeled amino acids, peptides, flourescently labeled peptides, proteins, antibody, saccharides, lipids, polynucleotides, PNA, LNA, RNA, DNA, flouroscently labeled DNA, DNA folded by self-assembly into 2D and 3D patterns, DNA nanostructures, Encapsulated polynucleotides, PNA, LNA, RNA, DNA, polymers, linear polymer, Polyhalogenated phenoxyalkyl derivatives, Mono-amides of iminodiacetic acid, Haloaromatic Binary Coding, Secondary Amine Binary Coding, Linear polymer comprising a nucleobase, Branched polymer, Inorganic, Quantum dots, Radio-frequency identification tags, Metal, Nanobarcodes, Metallic nanoparticles, Chromium coated glass, Shaped particles, nanorod, polystyrene microspheres, polystyrene nanoparticles, silica nanoparticles, Bead arrays, Rare earth-doped glass bars, Dye molecules attached to silver or gold nanoparticles, Dye molecules attached to gold nanoparticles via DNA oligonucleotides, Ceramic plates with a two-dimensional, laser-etched bar codes

### Embodiment 32

The method of Embodiments 30-31, wherein said tags comprise one or more nucleoside(s) selected from deoxyadenosine, deoxyguanosine, deoxythymidine, adenosine, guanosine, uridine, cytidine, and inosine, wherein said nucleosides are connected by an intermucleoside linker selected from the group consisting of a phospodiester bond, a phosphorothioate bond, a methylphosphonate bond, a phosphoramidate bond, a phosphotriester bond and a phosphodithioate bond

### Embodiment 33

The method of Embodiment 1, wherein said medium comprises a solvent with a molecular weight chosen from 2E+0 to 1E+1, 1E+1 to 3E+1, 3E+1 to 9E+1, 9E+1 to 3E+2, 3E+2 to 8E+2, 8E+2 to 2E+3, 2E+3 to 7E+3, 7E+3 to 2E+4, 2E+4 to 7E+4, 7E+4 to 2E+5, 2E+5 to 6E+5, 6E+5 to 2E+6, 2E+6 to 5E+6, 5E+6 to 2E+7, 2E+7 to 5E+7, 5E+7 to 1E+8

### Embodiment 34

The method of Embodiment 1, wherein said medium comprises a solvent with a number of atoms chosen from 2 to 4, 4 to 7, 7 to 11, 11 to 17, 17 to 27, 27 to 42, 42 to 68, 68 to 108, 108 to 172, 172 to 275, 275 to 440, 440 to 704, 704 to 1126, 1126 to 1802, 1802 to 2883, 2883 to 4612

### Embodiment 35

The method of Embodiment 1, wherein said medium comprises a solvent with a number of different atomic elements chosen from 1 to 2, 2 to 3, 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, 20 to 21, 21 to 22, 22 to 23, 23 to 24, 24 to 25

### Embodiment 36

The method of Embodiment 1, wherein said medium has a refractive index chosen from 1,0 to 2,0; 2,0 to 2,2; 2,2 to 2,3; 2,3 to 2,5; 2,5 to 2,6; 2,6 to 2,8; 2,8 to 2,9; 2,9 to 3,1; 3,1 to 3,2; 3,2 to 3,4; 3,4 to 3,5; 3,5 to 3,7; 3,7 to 3,8; 3,8 to 4,0; 4,0 to 4,1; 4,1 to 4,3; 4,3 to 4,4; 4,4 to 4,6; 4,6 to 4,7; 4,7 to 4,9; 4,9 to 5,0; 5,0 to 5,2; 5,2 to 5,3; 5,3 to 5,5

### Embodiment 37

The method of Embodiment 1, wherein said medium has a dielectric constant chosen from 2E+0 to 1E+1, 1E+1 to 3E+1, 3E+1 to 6E+1, 6E+1 to 2E+2, 2E+2 to 4E+2, 4E+2 to 1E+3, 1E+3 to 2E+3, 2E+3 to 6E+3, 6E+3 to 2E+4, 2E+4 to 4E+4, 4E+4 to 1E+5, 1E+5 to 2E+5, 2E+5 to 6E+5, 6E+5 to 1E+6, 1E+6 to 4E+6, 4E+6 to 9E+6

### Embodiment 38

The method of Embodiment 1, wherein said medium comprises a component with a charge chosen from −50 to −45, −45 to −40, −40 to −35, −35 to −30, −30 to −25, −25 to −20, −20 to −15, −15 to −10, −10 to −5, −5 to 0, 0 to 5, 5 to 10, 10 to 15, 15 to 20, 20 to 25, 25 to 30, 30 to 35, 35 to 40, 40 to 45, 45 to 50

### Embodiment 39

The method of Embodiment 1, wherein said medium has a pH chosen from 0,0 to 1,0; 1,0 to 2,0; 2,0 to 3,0; 3,0 to 4,0; 4,0 to 5,0; 5,0 to 6,0; 6,0 to 6,1; 6,1 to 6,2; 6,2 to 6,3; 6,3 to 6,4; 6,4 to 6,5; 6,5 to 6,6; 6,6 to 6,7; 6,7 to 6,8; 6,8 to 6,9; 6,9 to 7,0; 7,0 to 7,1; 7,1 to 7,2; 7,2 to 7,3; 7,3 to 7,4; 7,4 to 7,5; 7,5 to 7,6; 7,6 to 7,7; 7,7 to 7,8; 7,8 to 7,9; 7,9 to 8,0; 8,0 to 9,0; 9,0 to 10,0; 10,0 to 11,0; 11,0 to 12,0; 12,0 to 13,0; 13,0 to 14,0

### Embodiment 40

The method of Embodiment 1, wherein said medium comprises one or more pH buffering entities chosen from ACES [6.78; 6.1-7.5], acetate [4.76; 3.6-5.6], ADA [6.59; 6.0-7.2], ammonium hydroxide [9.25; 8.8-9.9], AMP (2-amino-2-methyl-1-propanol) [9.69; 8.7-10.4], AMPD (2-amino-2-methyl-1,3-propanediol) [8.80; 7.8-9.7], AMPSO [9.00; 8.3-9.7], BES [7.09; 6.4-7.8], BICINE [8.26; 7.6-9.0], bis-tris [6.46; 5.8-7.2], BIS-TRIS propane [6.80, 9.00; 6.3-9.5], borate [9.23, 12.74, 13.80; 8.5-10.2], CABS [10.70; 10.0-11.4], cacodylate [6.27; 5.0-7.4], CAPS [10.40; 9.7-11.1], CAPSO [9.60; 8.9-10.3], carbonate (pK1) [6.35; 6.0-8.0], carbonate (pK2) [10.33; 9.5-11.1], CHES [9.50; 8.6-10.0], citrate (pK1) [3.13; 2.2-6.5], citrate (pK2) [4.76; 3.0-6.2], citrate (pK3) [6.40; 5.5-7.2], DIPSO [7.52; 7.0-8.2], EPPS, HEPPS [8.00; 7.6-8.6], ethanolamine [9.50; 6.0-12.0], formate [3.75; 3.0-4.5], glycine (pK1) [2.35; 2.2-3.6], glycine (pK2) [9.78; 8.8-10.6], glycylglycine (pK1) [3.14; 2.5-3.8], glycylglycine (pK2) [8.25; 7.5-8.9], HEPBS [8.30; 7.6-9.0], HEPES [7.48; 6.8-8.2], HEPPSO [7.85; 7.1-8.5], histidine [1.70, 6.04, 9.09; 5.5-7.4], hydrazine [8.10; 7.5-10.0], imidazole [6.95; 6.2-7.8], malate (pK1) [3.40; 2.7-4.2], malate (pK2) [5.13; 4.0-6.0], maleate (pK1) [1.97; 1.2-2.6], maleate (pK2) [6.24; 5.5-7.2], MES [6.10; 5.5-6.7], methylamine [10.66; 9.5-11.5], MOBS [7.60; 6.9-8.3], MOPS [7.14; 6.5-7.9], MOPSO [6.87; 6.2-7.6], phosphate (pK1) [2.15; 1.7-2.9], phosphate (pK2) [7.20; 5.8-8.0], phosphate (pK3) [12.33;], piperazine (pK1) [5.33; 5.0-6.0], piperazine (pK2) [9.73; 9.5-9.8], piperidine [11.12; 10.5-12.0], PIPES [6.76; 6.1-7.5], POPSO [7.78; 7.2-8.5], propionate [4.87; 3.8-5.6], pyridine [5.23; 4.9-5.9], pyrophosphate [0.91, 2.10, 6.70, 9.32; 7.0-9.0], succinate (pK1) [4.21; 3.2-5.2], succinate (pK2) [5.64; 5.5-6.5], TABS [8.90; 8.2-9.6], TAPS [8.40; 7.7-9.1], TAPSO [7.61; 7.0-8.2], taurine (AES) [9.06; 8.4-9.6], TES [7.40; 6.8-8.2], tricine [8.05; 7.4-8.8], triethanolamine (TEA) [7.76; 7.0-8.3], Trizma (tris) [8.06; 7.5-9.0]

### Embodiment 41

The method of Embodiment 1, wherein said medium comprises one or more solvents chosen from Water, Acetal (1,1-Diethoxyethane), Acetic acid, Acetone, Acetonitrile, Acetylacetone, Acrylonitrile, Adiponitrile, Allyl alcohol, Allylamine, 2-Aminoisobutanol, Benzal, Benzaldehyde, Benzene, Benzonitrile, Benzyl Chloride, Bromochloromethane, Bromoform (Tribromomethane), Butyl acetate, Butyl alcohol, sec-Butyl alcohol, tert-Butyl alcohol, Butylamine, tert-Butylamine, Butyl methyl ketone, p-tert-Butyltoluene, g-Butyrolactone, Caprolactam, Carbon disulfide, Carbon tetrachloride, 1-Chloro-1,1-difluoroethane, Chlorobenzene, Chloroform, Chloropentafluoroethane, Cumene (Isopropylbenzene), Cyclohexane, Cyclohexanol, Cyclohexanone, Cyclohexylamine, Cyclopentane, Cyclopentanone, p-Cymene, cis-Decalin, trans-Decalin, Diacetone alcohol, 1,2-Dibromoethane, Dibromofluoromethane, Dibromomethane, 1,2-Dibromotetrafluoroethane, Dibutylamine, o-Dichlorobenzene, 1,1-Dichloroethane, 1,2-Dichloroethane, 1,1-Dichloroethylene, cis-1,2-Dichloroethylene, trans-1,2-Dichloroethylene, Dichloroethyl ether, Dichloromethane, 1,2-Dichloropropane, 1,2-Dichlorotetrafluoroethane, Diethanolamine, Diethylamine, Diethyl, Diethylene glycol, Diethylene glycol dimethyl ether, Diethylene glycol monoethyl ether, Diethylene glycol monoethyl ether acetate, Diethylene glycol monomethyl ether, Diethylenetriamine, Diethyl ether, Diisobutyl ketone, Diisopropyl ether, N,N-Dimethylacetamide, Dimethylamine, Dimethyl disulfide, N,N-Dimethylformamide, Dimethyl sulfoxide, 1,4-Dioxane, 1,3-Dioxolane, Dipentene, Epichlorohydrin, Ethanolamine (Glycinol), Ethyl acetate, Ethyl acetoacetate, Ethyl alcohol, Ethylamine, Ethylbenzene, Ethyl bromide, Ethyl Chloride, Ethylene Carbonate, Ethylenediamine, Ethylene glycol, Ethylene glycol diethyl ether, Ethylene glycol dimethyl ether, Ethylene glycol monobutyl ether, Ethylene glycol monoethyl ether, Ethylene glycol ethyl ether acetate, Ethylene glycol monomethyl ether, Ethylene glycol momomethyl ether acetate, Ethyl formate, Furan, Furfural, Furfuryl alcohol, Glycerol, Heptane, 1-Heptanol, Hexane, 1-Hexanol (Caproyl alcohol), Hexylene glycol, Hexyl methyl ketone, Isobutyl acetate, Isobutyl alcohol, Isobutylamine, Isopentyl acetate, Isophorone, Isopropyl acetate, Isopropyl alcohol, Isoquinoline, d-Limonene (Citrene), 2,6-Lutidine, Mesitylene, Mesityl oxide, Methyl acetate, Methylal, Methyl alcohol, Methylamine, Methyl benzoate, Methylcyclohexane, Methyl ethyl ketone, N-Methylformamide, Methyl formate, Methyl iodide, Methyl isobutyl ketone, Methyl isopentyl ketone, 2-Methylpentane, 4-Methyl-2-pentanol, Methyl pentyl ketone, Methyl propyl ketone, N-Methyl-2-pyrrolidone, Morpholine, Nitrobenzene, Nitroethane, Nitromethane, 1-Nitropropane, 2-Nitropropane, Octane, 1-Octanol, Pentachloroethane, Pentamethylene glycol, Pentane, 1-Pentanol, Pentyl acetate, 2-Picoline, a-Pinene, b-Pinene, Piperidine, Propanenitrile, Propyl acetate, Propyl alcohol, Propylamine, Propylbenzene, Propylene glycol, Pseudocumene, Pyridine, Pyrrole, Pyrrolidine, 2-Pyrrolidone, Quinoline, Styrene, Sulfolane, a-Terpinene, 1,1,1,2-Tetrachloro-2,2-difluoroethane, 1,1,2,2-Tetrachloro-1,2-difluoroethane, 1,1,1,2-Tetrachloroethane, 1,1,2,2-Tetrachloroethane, Tetrachloroethylene, Tetraethylene glycol, Tetrahydrofuran, 1,2,3,4-Tetrahydronaphthalene, Tetrahydropyran, Tetramethylsilane, Toluene, o-Toluidine, Triacetin, Tributylamine, 1,1,1-Trichloroethane, 1,1,2-Trichloroethane, Trichloroethylene, Trichlorofluoromethane, 1,1,2-Trichlorotrifluoroethane, Triethanolamine, Triethylamine, Triethylene glycol, Triethyl phosphate, Trimethylamine, Trimethylene glycol, Trimethyl phosphate, Veratrole, o-Xylene, m-Xylene, p-Xylene

### Embodiment 42

The method of Embodiment 1, wherein said medium comprises a repeat unit (X) of a material wherein the number of repeating units is chosen from 1 to 2, 2 to 3, 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, 20 to 21, 21 to 22, 22 to 23, 23 to 24, 24 to 25

### Embodiment 43

The method of Embodiment 1, wherein said medium comprises one ore more repeat units of a polymer where said polymer is chosen from Polyethylenes: Polyethylene (PE), Low density polyethylene (LDPE), High density polyethylene (HDPE), Linear low density polyethylene (LLDPE), Crosslinked polyethylene (XLPE), Ultra High Molecular Weight Polyethylene (UHMWPE). Other Polyolefins: Polypropylene (PP), Biaxially-oriented polypropylene, Polybutylene (PB), Polyisobutene (PIB) Polyacrylates: Polymethyl methacrylate (PMMA), polymethyl acrylate (PMA), hydroxyethyl methacrylate (HEMA), Sodium polyacrylate. Polystyrenes: Polystyrene (PS), High impact polystyrene (HIPS), Extruded polystyrene (XPS), Expanded Polystyrene Polyesters: Polyethylene terephthalate (PET). Polysulfones: Polysulfone (PSU), Polyarylsulfone (PAS), Polyethersulfone PES, Polyphenylsulfone (PPS). Polyamides: Polyamide (PA), polyphthalamide (PPA), Bismaleimide (BMI), urea formaldehyde (UF) Polyurethanes: Polyurethane (PU), Polyisocyanurate (PIR). Chloropolymers: Polyvinyl chloride (PVC), Polyvinylidene dichloride (PVDC). (Chloro)fluoropolymers: Fluoropolymer (FE), Polytetrafluoroethylene (PTFE), Polyvinylidene difluoride (PVDF), Polychlorotrifluoroethlyene (PCTFE), Ethylene chlorotrifluoroethlyene (ECTFE). Other Homopolymers: Polycarbonate (PC), Polylactic acid (PLA), Polyacrylamide (PAM), Polyetheretherketone (PEEK). Other Copolymers: Acrylonitrile butadiene styrene (ABS), Polybutadiene acrylonitrile (PBAN). Polymers suitable for use according to the present invention may also be selected from; BMI Bismaleimide, Cellulose acetate, Cellulose acetate butyrate, Cellulose acetate propionate, Cellulose propionate, Cellulosics, cyamelide, ECTFE Ethylene chlorotrifluoroethlyene, ETFE Ethylene TetrafluoroEthylene, Ethyl cellulose, FEP Fluorinated Ethylene Propylene, HDPE High density polyethylene, HEMA hydroxyethyl methacrylate, HEMA hydroxyethyl methacrylate, LDPE Low density polyethylene, LLDPE Linear low density polyethylene, Methyl methacrylate, PA Polyamide, PAM polyacrylamide, PAM polyacrylamide, PAN polyacrylonitrile, PAN polyacrylonitrile, PAS Polyarylsulfone, PB Polybutylene, PBAN polybutadiene acrylonitrile, PBAN polybutadiene acrylonitrile, PBT Polybutylene terephthalate, PCT Type, PCTA Type, PCTFE Polychlorotrifluoroethylene (Teflon), PCTG Type, PE Polyethylene, PEBA Polyether Block Amide elastomer, PES Polyethersulfone, PET Polyethylene terephthalate, PETG Type, PEX Crosslinked high density polyethylene, PFA Perfluoroalkoxyethylene, PIB Polyisobutene, PMA polymethyl acrylate, PMA polymethyl acrylate, PMMA polymethyl methacrylate, PMMA polymethyl methacrylate, Polyacrylate, Polyaryletherketones, polyurea, Polyvinyl, Polyvinyl acetate, Polyvinyl alcohol, Polyvinyl carbazole, Polyvinyl chloride, PP Polypropylene, PPA polyphthalamide, PPS Polyphenylsulfone, PSU Polysulfone, PTFE Polytetrafluoroethylene, PVDF Polyvinylidene fluoride, PVF Polyvinyl fluoride, Sodium polyacrylate, UF urea formaldehyde, UHMWPE Ultra high molecular weight polyethylene, XLPE Crosslinked Polyethylene, Alkyd, Allyl Diglycol Carbonate Monomer, Bismaleimide (BMI), DAIP (Diallyl Isophthalate), DAP (Diallyl Phthalate), DCPD Resin, Epoxy, Furan, Melamine, Melamine/Phenolic, Phenolic, Polybutandiene, Polyester (Unsaturated), Bulk Molding Compounds (BMC), Gel Coats, Granular Molding Compounds (GMC), Resins, Sheet Molding Compounds (SMC), Thick Molding Compounds (TMC), Polyimide (Thermosetting), Silicone, Urea, Urethane Chemicals, Vinyl Ester, Molding Compounds, ABS, acrylonitrile-butadiene-styrene copolymer; ACMA, acrylonitrile-methyl acrylate copolymer; AIBN, 2,2-azobisisobutyronitrile; ATRP, atom transfer radical polymerization; CNT, carbon nanotube; CPP, chlorinated polypropylene; DWCNT, double-walled carbon nanotube; EMMA, ethyl-methyl methacrylate copolymer; EPDM, ethylene-propylene-diene rubber; EVA, ethylene-vinyl acetate copolymer; EVOH, ethylene-vinyl alcohol copolymer; HDPE, high-density polyethylene; HMW, high molecular weight; LDPE, low density polyethylene; LMW, low molecular weight; MA, maleic anhydride; MBMA, methyl-butyl methacrylate copolymer; MDPE, medium density polyethylene; MEMA, methyl-ethyl methacrylate copolymer; MPTS, methacryloxypropyltrimethoxysilane; MWCNT, multi-walled carbon nanotube; NMP, nitroxide-mediated polymerization; NMR, nuclear magnetic resonance; P3HT, poly(3-hexylthiophene); P3OT, poly(3-octylthiophene); PA, poly-acetylene; PAA, poly(acrylic acid); PABS, poly(m-aminobenzene sulfonic acid); PAM, polyacrylamide; PAMAM, poly(amidoamine); PAN, polyacrylonitrile; PANI, polyaniline; Parmax, poly(benzoyl-1,4-phenylene)-co-(1,3-phenylene); PBA, polybutyl acrylate; PtBA, poly(tert-butyl acrylate); PBMA, poly(butyl methacrylate); PBO, poly(phenylenebenzobisoxazole); PBT, poly(butyl terephthalate); PC, polycarbonate; PCL, polycaprolactone; PDEAEMA, poly[2-(diethylamino)ethyl methacrylate]; PDI, polydispersity index; PDMEMA, poly[2-(dimethylamino)ethyl methacrylate]; PDMS, polydimethylsiloxane; PDPA, polydiphenylamime; PE, polyethylene; PEG, polyethyleneglycol; PEI, polyethyleneimine; PEMA, poly(ethyl methacrylate); PEO, polyethyleneoxide; PET, poly(ethyl terephthalate); PETI, phenylethynyl-terminated imide; PGMA, poly(glycerol monomethacrylate); PHEMA, poly(2-hydroxyethyl methacrylate); PHET, poly[3-(2-hydroxyethyl)-2,5-thienylene]; PHPMA, poly[N-(2-hydroxypropyl)methacrylamide]; PI, polyimide; PIMA, poly(imidazolium methacrylate); PLLA, poly(1-lactic acid); PLLA-g-AA, poly(1-lactic acid) grafted with poly(acrylic acid) chains; PMDMAS, poly[3-(N-(3-methacrylamidopropyl)-N,N-dimethyl)ammoniopropanatesulfonate]; PMMA, poly(methyl methacrylate); PMMAHEMA, poly[(methyl methacrylate)-co-(2-hydroxyethyl methacrylate)]; PmPV, poly(m-phenylenevinylene-co-2,5-dioctoxy-p-phenylenevinylene); PNIPAAm, poly(N-isopropylacrylamide); PP, polypropylene; PPE, poly(p-phenylene ethynylene); PPEI-EI, poly(propionylethylenimine-co-ethylimine); PPS, poly(phenylene sulfide); PPY, polypyrrole; PS, polystyrene; PSS, poly(sodium 4-styrenesulfonate); PSV, poly(styrene-co-p-(4-(4-vinylphenyl)-3-oxobutanol)); PTH, polythiophene; PU, polyurethane; PVA, poly(vinyl alcohol); PVAc, poly(vinyl acetate); PVAc-VA, poly(vinyl acetate-co-vinyl alcohol); PVC, poly(vinyl chloride); PVDF, poly(vinylidene fluoride); PVK, poly(N-vinyl carbazole); PVKV, poly(N-vinyl carbazole-co-p-(4-(4-vinylphenyl)-3-oxobutanol)); PVP, polyvinylpyrrolidone; P2VP, poly(2-vinylpyridine); P4VP, poly(4-vinylpyridine); SAN, styrene-acrylonitrile copolymer; SBA, styrene-butyl acrylate copolymer; SBBS, styrene-butadiene-butylene-styrene copolymer; SBR, styrene-butadiene rubber; SCMS, styrene-p-chloromethylstyrene copolymer; SE, silicone elastomer; SEC, size exclusion chromatography; SIBS, poly(styrene-b-isobutylene-b-styrene); SMA, styrene maleic anhydride copolymer; SWCNT, single-walled carbon nanotube; TDI, toluene diisocyanate; TEMPO, 2,2,6,6-tetramethylpiperidinyl-1-oxy; TGA, thermogravimetric analysis; THF, tetrahydrofuran; UHMWPE, ultra high molecular weight polyethylene; WBPU, waterborne polyurethane.

### Embodiment 44

The method of Embodiment 1, wherein said medium comprises a blocking agent chosen from skimmed milk, skimmed milk proteins, bacteria extract, mammalian cell extract, yeast extract, milk, cetyltrimethyl ammonium bromide (CTAB), hexadecyltrimethyl ammonium bromide (HTAB), an ionic molecule, such as a detergent, a non-ionic molecule, such as a nonionic detergent, a biomolecule, such as an oligonucleotide, such as a single- or double- or triple- or quadruple-stranded DNA or RNA or modified DNA or modified RNA or DNA mimic or RNA mimic, a polymer, such as a PEG chain, or one or more suitable polymer(s), such as a tag-binding polymer, an enzyme, a structural protein, a receptor.

### Embodiment 45

The method of Embodiment 1, wherein said surface or material comprises the following molecular weight (in daltons): 2E+0 to 1E+1, 1E+1 to 3E+1, 3E+1 to 9E+1, 9E+1 to 3E+2, 3E+2 to 8E+2, 8E+2 to 2E+3, 2E+3 to 7E+3, 7E+3 to 2E+4, 2E+4 to 7E+4, 7E+4 to 2E+5, 2E+5 to 6E+5, 6E+5 to 2E+6, 2E+6 to 5E+6, 5E+6 to 2E+7, 2E+7 to 5E+7, 5E+7 to 1E+8.

### Embodiment 46

The method of Embodiment 1, wherein said surface or material comprise the following number of atoms 2 to 4, 4 to 7, 7 to 11, 11 to 17, 17 to 27, 27 to 42, 42 to 68, 68 to 108, 108 to 172, 172 to 275, 275 to 440, 440 to 704, 704 to 1126, 1126 to 1802, 1802 to 2883, 2883 to 4612.

### Embodiment 47

The method of Embodiment 1, wherein said surface or material comprises the following number of different atomic elements 1 to 2, 2 to 3, 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, 20 to 21, 21 to 22, 22 to 23, 23 to 24, 24 to 25.

### Embodiment 48

The method of Embodiment 1, wherein said surface or material has a density (in grams per litre) chosen from 1E−2 to 1E−1, 1E−1 to 3E−1, 3E−1 to 9E−1, 9E−1 to 3E+0, 3E+0 to 8E+0, 8E+0 to 2E+1, 2E+1 to 7E+1, 7E+1 to 2E+2, 2E+2 to 7E+2, 7E+2 to 2E+3, 2E+3 to 6E+3, 6E+3 to 2E+4, 2E+4 to 5E+4, 5E+4 to 2E+5, 2E+5 to 5E+5, 5E+5 to 1E+6.

### Embodiment 49

The method of Embodiment 1, wherein said surface or material has a Young's modulus (in gigapascal) chosen from 1E−4 to 1E−3, 1E−3 to 3E−3, 3E−3 to 9E−3, 9E−3 to 3E−2, 3E−2 to 8E−2, 8E−2 to 2E−1, 2E−1 to 7E−1, 7E−1 to 2E+0, 2E+0 to 7E+0, 7E+0 to 2E+1, 2E+1 to 6E+1, 6E+1 to 2E+2, 2E+2 to 5E+2, 5E+2 to 2E+3, 2E+3 to 5E+3, 5E+3 to 1E+4, 1E+4 to 4E+4, 4E+4 to 1E+5.

### Embodiment 50

The method of Embodiment 1, wherein said surface or material has a tensile strength (in gigapascal) chosen from 1E−4 to 1E−3, 1E−3 to 3E−3, 3E−3 to 6E−3, 6E−3 to 2E−2, 2E−2 to 4E−2, 4E−2 to 1E−1, 1E−1 to 2E−1, 2E−1 to 6E−1, 6E−1 to 2E+0, 2E+0 to 4E+0, 4E+0 to 1E+1, 1E+1 to 2E+1, 2E+1 to 6E+1, 6E+1 to 1E+2, 1E+2 to 4E+2, 4E+2 to 9E+2, 9E+2 to 2E+3, 2E+3 to 6E+3, 6E+3 to 1E+4, 1E+4 to 4E+4, 4E+4 to 9E+4, 9E+4 to 2E+5, 2E+5 to 6E+5, 6E+5 to 1E+6, 1E+6 to 4E+6, 4E+6 to 9E+6.

### Embodiment 51

The method of Embodiment 1, wherein said surface or material is selected from an organic material, hair, nail, horn, ligaments, bone, cornea, teeth, fibrous cartilage, vitreous cells, intervertebral disc, womb, skin, intestines, heart membranes, membranes, stomach membrane, cartilage, chronodrocites, intervertebral cartilage, bone enamel, ligaments, tendons and tooth enamel, an organ, lung, heart, brain, skin, kidney, tooth material, bone material, tendon material, skin, hair, nails, a body, a biological surface, such as a vein, a biological macromolecule, such as a protein, such as a naturally occurring protein, such as a consensus sequence protein, a modified protein, such as a protein where one or more amino acids have been changed relative to the consensus sequence, Rubber, Latex, Plastic, Polymers, Co-polymers, Block co-polymers, Inorganic, Coal, Soot, Carbon black, Anthracite, Lignite, Carbon nanofibre, Carbon allotropes, Activated carbon, Powdered activated carbon, Granular activated carbon, Extruded activated carbon, Fullerenes, Buckyball, Buckypaper, Buckytube, Dodecahedrane, Endohedral fullerenes, Gedodesic carbon domes, Prismane, Carbon nanotube, Single-wall carbon nanotubes, Double-wall carbon nanotubes, Triple-wall carbon nanotube, Multi-wall carbon nanotubes, Pristine carbon nanotubes, Coated carbon nanotubes, Defect-free carbon nanotubes, Defect carbon nanotubes, Functionalized carbon nanotubes, Thiol, Hydroxyl, Carboxylic acid, Amine, Carbon nanotubes that contain gadolinium, Buckypaper, Fullerite, Ultrahard fullerite, Buckminsterfullerene, Graphene, Single-layer graphene, Double-layer graphene, Triple-layer graphene, Multi-layer graphene, Pristine graphene, Coated graphene, Defect-free graphene, Defect graphene, Functionalized graphene, Graphane, Flourinated graphene, Graphene oxide, Reduced graphene oxide, Buckyball clusters, Carbon megatubes, Carbon polymers, Carbon nano-onions, Carbon nanobuds, Fullerene rings, Glassy carbon, Diamond, Hyperdiamonds, Aggregated diamonds, Graphite, Pyrolytic graphite, Pencil lead, Lonsdaleite, Amorphous carbon, Carbon nanofoam, cornea, Metals, Transition metal, Lanthanides, Actinides, Rare earth elements, Platinum group metals (PGMs), Post-transition metals, (Al) aluminium, (Fe) steel and stainless steel, (Mo) molybdenum, (Cu) copper, (Ti) titanium, (Pt) platinum, (Au) gold, (Ni) nickel, (Pa) palladium, (Mn) manganese, (Ta) tantalum, (Cr) chromium, (Ag) silver, (Wo) Tungsten, Metalloids, Alloys, Stainless steel, Hardened steel, Brass, Amalgams, Intermetallics, Glasses, Glass fibre, Crystal, Ionic crystals, Covalent crystals, Non-covalent crystals, Non-covalent crystal, Crystalline materials, BK7, sapphire, fused silica, Glue, Ceramics, alumina, zirconia, machinable ceramics, green ceramics, PZT, silicon nitride, tungsten carbide, alumina (Al2O3), silicon nitrides (bulk and thin film), silicon carbides, lithium niobates, zirconia, metallised ceramics, Polymers & plastics, polyimide, PTFE, PMMA, Kapton, Vespel, Cirlex, ABS, polyimides (kapton, upilex, etc), polyamides, polycarbonates (PC/lexan), polyesters (PET/mylar, melinex, dacron, PEN/teonex), polyethylenes (LDPE, HDPE), polypropylenes (PP), styrenics (polystyrenes/PS, acrylonitriles/ABS), vinyls (PVC, nylon), acrylics (PMMA/perspex, plexiglas), fluoroplastics (PTFE/teflon, FEP, PFA, PVDF), polysulphones (PES), ketones (PEEK), polyurethanes, barrier resins (PVA/polyvinyl alcohol), epoxy resins (FR4), silicone resins, elastomes (PDMS), biopolymers (wood, cellulose, stacrch based), conductive polymers (Pedot:PSS/baytron, orgacon, TIPS pentacene), light emitting polymers (white LEP, etc), copolymers, metalised polymers, Elastomers, Fibres, Composite materials, Wood, Bone, Tooth enamel, Tooth cementum, Tooth dentine, Sol-gel, Mineral, Organic-Inorganic, Organic-Inorganic hybrids, Polymer hybrids of poly (vinyl alcohol) and silica gel Embodiment 52. The method of Embodiment 1, wherein said surface or material comprises 1, 2, 3, 4, 5, 6, 7, 8, 9, or 10 different atomic elements chosen from Hydrogen (H), Helium (He), Lithium (Li), Beryllium (Be), Boron (B), Carbon (C), Nitrogen (N), Oxygen (O), Fluorine (F), Neon (Ne), Sodium (Na), Magnesium (Mg), Aluminium (Al), Silicon (Si), Phosphorus (P), Sulfur (S), Chlorine (Cl), Argon (Ar), Potassium (K), Calcium (Ca), Scandium (Sc), Titanium (Ti), Vanadium (V), Chromium (Cr), Manganese (Mn), Iron (Fe), Cobalt (Co), Nickel (Ni), Copper (Cu), Zinc (Zn), Gallium (Ga), Germanium (Ge), Arsenic (As), Selenium (Se), Bromine (Br), Krypton (Kr), Rubidium (Rb), Strontium (Sr), Yttrium (Y), Zirconium (Zr), Niobium (Nb), Molybdenum (Mo), Technetium (Tc), Ruthenium (Ru), Rhodium (Rh), Palladium (Pd), Silver (Ag), Cadmium (Cd), Indium (In), Tin (Sn), Antimony (Sb), Tellurium (Te), Iodine (I), Xenon (Xe), Caesium (Cs), Barium (Ba), Lanthanum (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu), Hafnium (Hf), Tantalum (Ta), Tungsten (W), Rhenium (Re), Osmium (Os), Iridium (Ir), Platinum (Pt), Gold (Au), Mercury (Hg), Thallium (Tl), Lead (Pb), Bismuth (Bi), Polonium (Po), Astatine (At), Radon (Rn), Francium (Fr), Radium (Ra), Actinium (Ac), Thorium (Th), Protactinium (Pa), Uranium (U), Neptunium (Np), Plutonium (Pu), Americium (Am), Curium (Cm), Berkelium (Bk), Californium (Cf), Einsteinium (Es), Fermium (Fm), Mendelevium (Md), Nobelium (No), Lawrencium (Lr), Rutherfordium (Rf), Dubnium (Db), Seaborgium (Sg), Bohrium (Bh), Hassium (Hs), Meitnerium (Mt), Darmstadtium (Ds), Roentgenium (Rg), Copernicium (Cn), Ununtrium (Uut), Ununquadium (Uuq), Ununpentium (Uup), Ununhexium (Uuh), Ununseptium (Uus), Ununoctium (Uuo)

### Embodiment 53

The method of Embodiment 1, wherein said surface or material comprises Quantum dots, Nanostructures, Colloidal gold, Cup, Nanocup, Stacked carbon nanocup, Particle, Nanoparticle, Silver nanoparticle, Gold nanoparticle, Microparticle, Tube, Carbon tube, Metallic tube, Metallic nanotube, Metallic carbon nanotube, Nanotube, nanotubes made of carbon, zinc, gold, silver, aluminum, Microtube, Film, Fibre, Carbon fibre, Vitreous fibre, Glass fibre, Glass nanofibre, Metallic fibre, Metallic carbon nanofibre, Nanofibre, Microfibre, Particle, Nanoparticle, Microparticle, Ribbon, Nanoribbon, Carbon nanoribbon, Microribbon, Layer, monolayer, Fullerenere monolayer, Multilayer, Fullerene multilayer, Sphere, Nanosphere, Sheet, Nanosheet, Foam, Rod, Nanorods, Aggregated diamond nanorod, Knot, Nanorod, Wall, Carbon nanowall, Silver nanoparticles, Iron nanoparticles, Platinum nanoparticles, Nanotubes, ZnO, CeO2, CNT, gold, Particles, surfaces, nanoclusters, carbon nanofiber, diamond, diamond film, Bamboo-structured nanotubes, Filter, Pore size, Pore, Membrane, Nanomembrane, coordination compound, An assembly

### Embodiment 54

The method of Embodiment 1, wherein said surface or material comprises Electrode, Semiconductor, Diode, Electrode, Conductors, Capacitator, Battery, Anode, Cathode, Sensor, Super hard materials, diamond, silicon nitride, tungsten carbide, Dielectrics, glasses, borosilicate (Pyrex), bulk fused silica, crown, soda lime, doped glasses, SiO2, synthetic diamond, sapphire, fibres, metalised dielectrics, Semiconductors, wafers, silicon, germanium, gallium arsenide, gallium nitride, Catalyst, Insulators

### Embodiment 55

The method of Embodiment 1, wherein said surface or material comprises a chiral vector chosen from (0,0); (1,0); (2,0); (3,0); (4,0); (5,0); (6,0); (7,0); (8,0); (9,0); (10,0); (11,0); (12,0); (13,0); (14,0); (15,0); (16,0); (17,0); (18,0); (19,0); (20,0); (0,1); (1,1); (2,1); (3,1); (4,1); (5,1); (6,1); (7,1); (8,1); (9,1); (10,1); (11,1); (12,1); (13,1); (14,1); (15,1); (16,1); (17,1); (18,1); (19,1); (20,1); (0,2); (1,2); (2,2); (3,2); (4,2); (5,2); (6,2); (7,2); (8,2); (9,2); (10,2); (11,2); (12,2); (13,2); (14,2); (15,2); (16,2); (17,2); (18,2); (19,2); (20,2); (0,3); (1,3); (2,3); (3,3); (4,3); (5,3); (6,3); (7,3); (8,3); (9,3); (10,3); (11,3); (12,3); (13,3); (14,3); (15,3); (16,3); (17,3); (18,3); (19,3); (20,3); (0,4); (1,4); (2,4); (3,4); (4,4); (5,4); (6,4); (7,4); (8,4); (9,4); (10,4); (11,4); (12,4); (13,4); (14,4); (15,4); (16,4); (17,4); (18,4); (19,4); (20,4); (0,5); (1,5); (2,5); (3,5); (4,5); (5,5); (6,5); (7,5); (8,5); (9,5); (10,5); (11,5); (12,5); (13,5); (14,5); (15,5); (16,5); (17,5); (18,5); (19,5); (20,5); (0,6); (1,6); (2,6); (3,6); (4,6); (5,6); (6,6); (7,6); (8,6); (9,6); (10,6); (11,6); (12,6); (13,6); (14,6); (15,6); (16,6); (17,6); (18,6); (19,6); (20,6); (0,7); (1,7); (2,7); (3,7); (4,7); (5,7); (6,7); (7,7); (8,7); (9,7); (10,7); (11,7); (12,7); (13,7); (14,7); (15,7); (16,7); (17,7); (18,7); (19,7); (20,7); (0,8); (1,8); (2,8); (3,8); (4,8); (5,8); (6,8); (7,8); (8,8); (9,8); (10,8); (11,8); (12,8); (13,8); (14,8); (15,8); (16,8); (17,8); (18,8); (19,8); (20,8); (0,9); (1,9); (2,9); (3,9); (4,9); (5,9); (6,9); (7,9); (8,9); (9,9); (10,9); (11,9); (12,9); (13,9); (14,9); (15,9); (16,9); (17,9); (18,9); (19,9); (20,9); (0,10); (1,10); (2,10); (3,10); (4,10); (5,10); (6,10); (7,10); (8,10); (9,10); (10,10); (11,10); (12,10); (13,10); (14,10); (15,10); (16,10); (17,10); (18,10); (19,10); (20,10); (0,11); (1,11); (2,11); (3,11); (4,11); (5,11); (6,11); (7,11); (8,11); (9,11); (10,11); (11,11); (12,11); (13,11); (14,11); (15,11); (16,11); (17,11); (18,11); (19,11); (20,11); (0,12); (1,12); (2,12); (3,12); (4,12); (5,12); (6,12); (7,12); (8,12); (9,12); (10,12); (11,12); (12,12); (13,12); (14,12); (15,12); (16,12); (17,12); (18,12); (19,12); (20,12); (0,13); (1,13); (2,13); (3,13); (4,13); (5,13); (6,13); (7,13); (8,13); (9,13); (10,13); (11,13); (12,13); (13,13); (14,13); (15,13); (16,13); (17,13); (18,13); (19,13); (20,13); (0,14); (1,14); (2,14); (3,14); (4,14); (5,14); (6,14); (7,14); (8,14); (9,14); (10,14); (11,14); (12,14); (13,14); (14,14); (15,14); (16,14); (17,14); (18,14); (19,14); (20,14); (0,15); (1,15); (2,15); (3,15); (4,15); (5,15); (6,15); (7,15); (8,15); (9,15); (10,15); (11,15); (12,15); (13,15); (14,15); (15,15); (16,15); (17,15); (18,15); (19,15); (20,15); (0,16); (1,16); (2,16); (3,16); (4,16); (5,16); (6,16); (7,16); (8,16); (9,16); (10,16); (11,16); (12,16); (13,16); (14,16); (15,16); (16,16); (17,16); (18,16); (19,16); (20,16); (0,17); (1,17); (2,17); (3,17); (4,17); (5,17); (6,17); (7,17); (8,17); (9,17); (10,17); (11,17); (12,17); (13,17); (14,17); (15,17); (16,17); (17,17); (18,17); (19,17); (20,17); (0,18); (1,18); (2,18); (3,18); (4,18); (5,18); (6,18); (7,18); (8,18); (9,18); (10,18); (11,18); (12,18); (13,18); (14,18); (15,18); (16,18); (17,18); (18,18); (19,18); (20,18); (0,19); (1,19); (2,19); (3,19); (4,19); (5,19); (6,19); (7,19); (8,19); (9,19); (10,19); (11,19); (12,19); (13,19); (14,19); (15,19); (16,19); (17,19); (18,19); (19,19); (20,19); (0,20); (1,20); (2,20); (3,20); (4,20); (5,20); (6,20); (7,20); (8,20); (9,20); (10,20); (11,20); (12,20); (13,20); (14,20); (15,20); (16,20); (17,20); (18,20); (19,20); (20,20).

### Embodiment 59

The method of Embodiment 1, wherein said manipulation comprises Heating, Cooling, Subjecting to a magnetic field, Subjecting to electromagnetic radiation, Subjecting to ultraviolet light, Subjecting to laser light, Subjecting to mechanical rocking, Subjecting to suction, Subjecting to high pressure, Subjecting to low pressure

### Embodiment 60

The method of Embodiment 1, wherein said partitioning comprises Adsorption Chromatography, Affinity capillary electrophoresis (ACE), Affinity screening—Mass Spectroscopy, Affinity selection on immobilized surface, Affinity selection on surface in solution, Affinity selection on surfaces, Affinity chromatography, Chromatography, Dialysis, Exclusion chromatography, Gradient Elution, Ion-Exchange Chromatography, Isothermal Chromatography, Kinetic screening, e.g. screening for on-rate or off-rate, Ligand density gradient, Normal-Phase Chromatography, Partition Chromatography, Post-Column Derivatization, Programmed-Flow Chromatography (Flow Programming), Programmed-Pressure Chromatography (Pressure Programming), Programmed-Temperature Chromatography (Temperature Programming), Pyrolysis-Gas Chromatography, Reaction Chromatography, Reversed-Phase Chromatography, Frontal affinity chromatography, Gel electrophoresis, Screening for affinity by size exclusion chromatography.

### Embodiment 61

The method of Embodiment 1, wherein said characteristics chosen from Molecular weight, Number of hydrogen bond donors, Number of hydrogen bond acceptors, Number of rotatable bonds, charge, density, melting point, affinity, NMR spectrum, Raman spectrum, mass spectrum, absorbance, sequence, fragment sequence, amino acid sequence, nucleotide sequence.

### Embodiment 62

A composition comprising carbon nanotubes, one or more repeat units, an organic solvent, and one or more molecule libraries.

### Embodiment 63

A composition comprising graphene, one or more repeat units, an organic solvent, and one or more molecule libraries.

1. A method for identification of molecules with desired characteristics, comprising the steps of:
A. Providing a library of molecules of the general composition M(n)-L(k)-T(s): 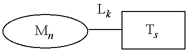
where n=1 to 1E+9, k=1 to 1E+9, and s=1 to 1E+9,
and where M is a library molecule, T is a tag that may be used to identify the library molecule M to which it is attached, and L is a linker that connects M and T;
B. Providing a medium; wherein said medium comprises a soluble polymer comprising two or more repeat units or a soluble mimic of a polymer selected from Polyethylene, Polyolefins such as Polypropylenes, Polybutylenes, Polyisobutenes (PIB), Polyacrylates such as Polymethyl methacrylate (PMMA), polymethyl acrylate (PMA), hydroxyethyl methacrylate (HEMA), Sodium polyacrylate, Polystyrenes, Polyesters, Polyethylene terephthalate (PET), Polysulfones such as Polysulfone (PSU), Polyarylsulfone (PAS), Polyethersulfone PES, Polyphenylsulfone (PPS), Polyamides such as Polyamide (PA), polyphthalamide (PPA), Bismaleimide (BMI), urea formaldehyde (UF); Polyurethanes such as Polyurethane (PU), Polyisocyanurate (PIR), Chloropolymers such as Polyvinyl chloride (PVC), Polyvinylidene dichloride (PVDC), (Chloro)fluoropolymers such as Fluoropolymer (FE), Polytetrafluoroethylene (PTFE), Polyvinylidene difluoride (PVDF), Polychlorotrifluoroethylene (PCTFE), Ethylene chlorotrifluoroethylene (ECTFE), Polycarbonate (PC), Polylactic acid (PLA), Polyacrylamide (PAM), Polyetheretherketone (PEEK), Acrylonitrile butadiene styrene (ABS), and Polybutadiene acrylonitrile (PBAN),
wherein said medium comprises one or more solvents selected from Water, Acetal (1,1-Diethoxyethane), Acetic acid, Acetone, Acetonitrile, Acetylacetone, Acrylonitrile, Adiponitrile, Allyl alcohol, Allylamine, 2-Aminoisobutanol, Benzal, Benzaldehyde, Benzene, Benzonitrile, Benzyl Chloride, Bromochloromethane, Bromoform (Tribromomethane), Butyl acetate, Butyl alcohol, sec-Butyl alcohol, tert-Butyl alcohol, Butylamine, tert-Butylamine, Butyl methyl ketone, p-tert-Butyltoluene, g-Butyrolactone, Caprolactam, Carbon disulfide, Carbon tetrachloride, 1-Chloro-1,1-difluoroethane, Chlorobenzene, Chloroform, Chloropentafluoroethane, Cumene (Isopropylbenzene), Cyclohexane, Cyclohexanol, Cyclohexanone, Cyclohexylamine, Cyclopentane, Cyclopentanone, p-Cymene, cis-Decalin, trans-Decalin, Diacetone alcohol, 1,2-Dibromoethane, Dibromofluoromethane, Dibromomethane, 1,2-Dibromotetrafluoroethane, Dibutylamine, o-Dichlorobenzene, 1,1-Dichloroethane, 1,2-Dichloroethane, 1,1-Dichloroethylene, cis-1,2-Dichloroethylene, trans-1,2-Dichloroethylene, Dichloroethyl ether, Dichloromethane, 1,2-Dichloropropane, 1,2-Dichlorotetrafluoroethane, Diethanolamine, Diethylamine, Diethyl, Diethylene glycol, Diethylene glycol dimethyl ether, Diethylene glycol monoethyl ether, Diethylene glycol monoethyl ether acetate, Diethylene glycol monomethyl ether, Diethylenetriamine, Diethyl ether, Diisobutyl ketone, Diisopropyl ether, N,N-Dimethylacetamide, Dimethylamine, Dimethyl disulfide, N,N-Dimethylformamide, Dimethyl sulfoxide, 1,4-Dioxane, 1,3-Dioxolane, Dipentene, Epichlorohydrin, Ethanolamine (Glycinol), Ethyl acetate, Ethyl acetoacetate, Ethyl alcohol, Ethylamine, Ethylbenzene, Ethyl bromide, Ethyl Chloride, Ethylene Carbonate, Ethylenediamine, Ethylene glycol, Ethylene glycol diethyl ether, Ethylene glycol dimethyl ether, Ethylene glycol monobutyl ether, Ethylene glycol monoethyl ether, Ethylene glycol ethyl ether acetate, Ethylene glycol monomethyl ether, Ethylene glycol momomethyl ether acetate, Ethyl formate, Furan, Furfural, Furfuryl alcohol, Glycerol, Heptane, 1-Heptanol, Hexane, 1-Hexanol (Caproyl alcohol), Hexylene glycol, Hexyl methyl ketone, Isobutyl acetate, Isobutyl alcohol, Isobutylamine, Isopentyl acetate, Isophorone, Isopropyl acetate, Isopropyl alcohol, Isoquinoline, d-Limonene (Citrene), 2,6-Lutidine, Mesitylene, Mesityl oxide, Methyl acetate, Methylal, Methyl alcohol, Methylamine, Methyl benzoate, Methylcyclohexane, Methyl ethyl ketone, N-Methylformamide, Methyl formate, Methyl iodide, Methyl isobutyl ketone, Methyl isopentyl ketone, 2-Methylpentane, 4-Methyl-2-pentanol, Methyl pentyl ketone, Methyl propyl ketone, N-Methyl-2-pyrrolidone, Morpholine, Nitrobenzene, Nitroethane, Nitromethane, 1-Nitropropane, 2-Nitropropane, Octane, 1-Octanol, Pentachloroethane, Pentamethylene glycol, Pentane, 1-Pentanol, Pentyl acetate, 2-Picoline, a-Pinene, b-Pinene, Piperidine, Propanenitrile, Propyl acetate, Propyl alcohol, Propylamine, Propylbenzene, Propylene glycol, Pseudocumene, Pyridine, Pyrrole, Pyrrolidine, 2-Pyrrolidone, Quinoline, Styrene, Sulfolane, a-Terpinene, 1,1,1,2-Tetrachloro-2,2-difluoroethane, 1,1,2,2-Tetrachloro-1,2-difluoroethane, 1,1,1,2-Tetrachloroethane, 1,1,2,2-Tetrachloroethane, Tetrachloroethylene, Tetraethylene glycol, Tetrahydrofuran, 1,2,3,4-Tetrahydronaphthalene, Tetrahydropyran, Tetramethylsilane, Toluene, o-Toluidine, Triacetin, Tributylamine, 1,1,1-Trichloroethane, 1,1,2-Trichloroethane, Trichloroethylene, Trichlorofluoromethane, 1,1,2-Trichlorotrifluoroethane, Triethanolamine, Triethylamine, Triethylene glycol, Triethyl phosphate, Trimethylamine, Trimethylene glycol, Trimethyl phosphate, Veratrole, o-Xylene, m-Xylene, p-Xylene
C. Providing a surface or material selected from carbon nanotubes, multi-walled carbon nanotubes, single-walled carbon nanotubes, functionalized carbon nanotubes, carbon nanofibres, carbon nanothreads, fullerenes, graphene, functionalized graphene, graphene oxide, graphyne, reduced graphyne, graphane, metal oxides such as Cu₂O, ZnO, metals such as gold, chromium, ceramics, semiconductors, boron nitride, boron nitride nanotubes, functionalized boron nitride, a polymer chosen from Polyethylenes, Polyolefins such as Polypropylenes, Polybutylenes, Polyisobutenes (PIB), Polyacrylates such as Polymethyl methacrylate (PMMA), polymethyl acrylate (PMA), hydroxyethyl methacrylate (HEMA), Sodium polyacrylate, Polystyrenes, Polyesters, Polyethylene terephthalate (PET), Polysulfones such as Polysulfone (PSU), Polyarylsulfone (PAS), Polyethersulfone PES, Polyphenylsulfone (PPS), Polyamides such as Polyamide (PA), polyphthalamide (PPA), Bismaleimide (BMI), urea formaldehyde (UF); Polyurethanes such as Polyurethane (PU), Polyisocyanurate (PIR), Chloropolymers such as Polyvinyl chloride (PVC), Polyvinylidene dichloride (PVDC), (Chloro)fluoropolymers such as Fluoropolymer (FE), Polytetrafluoroethylene (PTFE), Polyvinylidene difluoride (PVDF), Polychlorotrifluoroethylene (PCTFE), Ethylene chlorotrifluoroethylene (ECTFE), Polycarbonate (PC), Polylactic acid (PLA), Polyacrylamide (PAM), Polyetheretherketone (PEEK), Acrylonitrile butadiene styrene (ABS), and Polybutadiene acrylonitrile (PBAN);
D. Combining said library, medium, surface and/or material;
E. Optionally performing one or more manipulations of said library, medium, surface and/or material;
F. Partitioning or isolating a fraction of said library;
G. Optionally amplifying or copying the whole or a part of one or more molecules;
H. Optionally identifying one or more characteristics of one or more library molecules;
wherein
steps A, B and C are performed simultaneously or sequentially in any possible order, before step D;
wherein steps E, F, G and H are performed simultaneously or sequentially in any possible order after step D; and
wherein steps A, B, C, D, E, F, G, and H are performed one or more times.

2. The method of claim 1, wherein said polymer, repeat unit or mimic structurally or functionally resembles a repeat unit of the surface or material.

3. The method according to claim 1, wherein said library molecule M is selected from the group comprising small molecules, macrocycles, amino acids, peptides, macrocyclic peptides, proteins, carbohydrates, lipids, nucleic acids and mixed organic/inorganic molecules.

4. The method according to claim 3, wherein said library molecules M are selected from amino acids selected from L-amino acids, D-amino acids, alpha amino acids, beta amino acids, gamma amino acids, essential amino acids, non-essential amino acids, imino acids, N-substituted L-amino acids, N-substituted D-amino acids, N-substituted alpha amino acids, N-substituted beta amino acids, N-substituted gamma amino acids, N-substituted essential amino acids, N-substituted non-essential amino acids, and N-substituted imino acids; peptides comprising L-amino acids, D-amino acids, alpha amino acids, beta amino acids, gamma amino acids, essential amino acids, non-essential amino acids, imino acids, N-substituted L-amino acids, N-substituted D-amino acids, N-substituted alpha amino acids, N-substituted beta amino acids, N-substituted gamma amino acids, N-substituted essential amino acids, N-substituted non-essential amino acids, and/or N-substituted imino acids; proteins selected from antibodies, antibody fragments, VhH domains, V-NAR domains, VH domains, VL domains, Camel Igs, IgNAR, IgG, Fab, Fab2, Fab3, Bis-scFv, Minibody (bivalent), and scFV, Triabody, Diabody, Tetrabody, and enzymes; carbohydrates selected from linear carbohydrates, branched carbohydrates, monosaccharides, disaccharides, oligosaccharide, and polysaccharides; lipids selected from sterols, fatty acids, waxes, monoglycerides, diglycerides, phospholipids, fatty acyls, glycerolipids, glycerophospholipids, sphingolipids, saccharolipids, and polyketides; nucleic acids selected from RNA, mRNA, rRNA, tRNA, tmRNA, snRNA, snoRNA, scaRNA, gRNA, aRNA, crRNA, ncRNA; small molecules, macrocycles, and mixed organic/inorganic molecules, wherein said library molecules M are preferably selected from small molecules, macrocycles and peptides.

5. The method according to claim 1, wherein said tags T comprise poly-amino acids, peptides, proteins, antibodies, saccharides, lipids, polynucleotides, PNA, LNA, RNA, DNA, polymers, Polyhalogenated phenoxyalkyl derivatives, Mono-amides of iminodiacetic acid, metals, Chromium coated glass, polystyrene microspheres, polystyrene nanoparticles, silica nanoparticles, Rare earth-doped glass bars, Dye molecules attached to silver or gold nanoparticles, Dye molecules attached to gold nanoparticles via DNA oligonucleotides, Ceramic plates, nucleosides selected from deoxyadenosine, deoxyguanosine, deoxythymidine, adenosine, guanosine, uridine, cytidine, and inosine, wherein said nucleosides are connected by an internucleoside linker selected from the group consisting of a phosphodiester bond, a phosphorothioate bond, a methylphosphonate bond, a phosphoramidate bond, a phosphotriester bond and a phosphodithioate bond, said tags preferably being selected from DNA and RNA.

6. The method according to claim 1, wherein said linker L is selected carbohydrates and substituted carbohydrates, polyvinyl, acetylene or polyacetylene, aryl/hetaryl and substituted aryl/hetaryl, ethers and polyethers such as e.g. polyethylenglycol and substituted polyethers, amines, polyamines and substituted polyamines, single- and double-stranded oligonucleotides, polyamides, natural and unnatural polypeptides, monomer units such as a peptide, protein, carbohydrates and substituted carbohydrates, a nucleotide, ethylenglycol; 1,3-propylenglycol; 1,4-propylenglycol; 1,5-pentylenglycol, polyvinyl, acetylene or polyacetylene, aryl/hetaryl and substituted aryl/hetaryl, and any combination thereof.

7. The method according to claim 1, wherein said medium comprises one or more pH buffering entities.

8. The method according to claim 1, wherein said manipulation of step E comprises manipulation in the form of one or more of heating, cooling, subjecting to a magnetic field, subjecting to electromagnetic radiation, subjecting to ultraviolet light, subjecting to laser light, subjecting to mechanical rocking, subjecting to suction, subjecting to high pressure, and subjecting to low pressure.

9. The method according to claim 1, wherein said partitioning of step F comprises partitioning in the form of one or more of adsorption chromatography, affinity capillary electrophoresis (ACE), affinity screening—mass spectroscopy, affinity selection on immobilized surface, affinity selection on surface in solution, affinity selection on surfaces, affinity chromatography, chromatography, dialysis, exclusion chromatography, gradient elution, ion-exchange chromatography, isothermal chromatography, kinetic screening, e.g. screening for on-rate or off-rate, ligand density gradient, normal-phase chromatography, partition chromatography, post-column derivatization, programmed-flow chromatography (Flow Programming), programmed-pressure chromatography (Pressure Programming), programmed-temperature chromatography (Temperature Programming), pyrolysis-gas chromatography, reaction chromatography, reversed-phase chromatography, frontal affinity chromatography, gel electrophoresis, screening for affinity by size exclusion chromatography, and sonication.

10. The method according to claim 1, wherein said characteristics of the library molecules are chosen from molecular weight, atomic composition, number of hydrogen bond donors, number of hydrogen bond acceptors, number of atoms, number of rotatable bonds, charge, calculated logarithm of the octanol-water partition coefficient, polar surface area, total number of molecules, affinity and molecule state.

11. A kit for use in the method according to claim 1 comprising fullerenes, such as carbon nanotubes and/or graphene, and a molecule library, wherein said kit further comprises an organic solvent and/or a soluble polymer selected from Polyethylene, Polyolefins such as Polypropylenes, Polybutylenes, Polyisobutenes (PIB), Polyacrylates such as Polymethyl methacrylate (PMMA), polymethyl acrylate (PMA), hydroxyethyl methacrylate (HEMA), Sodium polyacrylate, Polystyrenes, Polyesters, Polyethylene terephthalate (PET), Polysulfones such as Polysulfone (PSU), Polyarylsulfone (PAS), Polyethersulfone PES, Polyphenylsulfone (PPS), Polyamides such as Polyamide (PA), polyphthalamide (PPA), Bismaleimide (BMI), urea formaldehyde (UF); Polyurethanes such as Polyurethane (PU), Polyisocyanurate (PIR), Chloropolymers such as Polyvinyl chloride (PVC), Polyvinylidene dichloride (PVDC), (Chloro)fluoropolymers such as Fluoropolymer (FE), Polytetrafluoroethylene (PTFE), Polyvinylidene difluoride (PVDF), Polychlorotrifluoroethylene (PCTFE), Ethylene chlorotrifluoroethylene (ECTFE), Polycarbonate (PC), Polylactic acid (PLA), Polyacrylamide (PAM), Polyetheretherketone (PEEK), Acrylonitrile butadiene styrene (ABS), and Polybutadiene acrylonitrile (PBAN).

12. The kit according to claim 11, wherein said molecule library is a phage-encoded library.

13. The kit according to claim 11, wherein said molecule library is a DNA-encoded small molecule or macrocyclic library.
